(19) United States
(12) Patent Application Publication
(10) Pub. No.: US 2026/0250352 A1
(43) Pub. Date: Aug. 27, 2026

(54) Methods and Uses of TACI-FC Fusion Immunomodulatory Protein

(71) Applicant: Alpine Immune Sciences, Inc., Seattle, WA (US)

(72) Inventors: Stanford L. Peng, Seattle, WA (US); Stacey Dillon, Seattle, WA (US); Jing Yang, Seattle, WA (US); Rupert Davies, Seattle, WA (US)

(73) Assignee: Alpine Immune Sciences, Inc., Seattle, WA (US)

(21) Appl. No.: 19/118,281

(22) PCT Filed: Oct. 3, 2023
(86) PCT No.: PCT/US2023/075875

§ 371 Date: Apr. 3, 2025

Related U.S. Application Data
(60) Provisional application No. 63/378,361, filed on Oct. 4, 2022.
(60) Provisional application No. 63/382,094, filed on Nov. 2, 2022.
(60) Provisional application No. 63/383,243, filed on Nov. 10, 2022.
(60) Provisional application No. 63/385,948, filed on Dec. 2, 2022.
(60) Provisional application No. 63/483,936, filed on Feb. 8, 2023.
(60) Provisional application No. 63/486,946, filed on Feb. 24, 2023.
(60) Provisional application No. 63/491,526, filed on Mar. 21, 2023.
(60) Provisional application No. 63/497,691, filed on Apr. 21, 2023.
(60) Provisional application No. 63/502,611, filed on May 16, 2023.
(60) Provisional application No. 63/505,053, filed on May 30, 2023.
(60) Provisional application No. 63/581,609, filed on Sep. 8, 2023.

(51) Int. Cl. C07K 14/705; A61K 9/00; A61K 38/00; A61K 45/06; A61P 37/06

(52) U.S. Cl. CPC C07K 14/70578; A61K 9/0019; A61K 45/06; A61P 37/06; A61K 38/00; C07K 2319/30

(57) ABSTRACT

Provided herein are methods of treatment and uses involving an immunomodulatory TACI-Fc fusion protein that exhibits neutralizing activity of BAFF and APRIL (or BAFF/APRIL heterotrimers). The provided TACI-Fc protein may include variant domains of Transmembrane Activator and CAML Interactor (TACI). The methods and uses provide therapeutic utility for a variety of immunological diseases, disorders or conditions, such as B cell-mediated diseases, disorder or conditions.

### CROSS-REFERENCE TO RELATED APPLICATIONS

This application claims priority to U.S. Provisional Application No. 63/378,361, filed Oct. 4, 2022, U.S. Provisional Application No. 63/382,094, filed Nov. 2, 2022, U.S. Provisional Application No. 63/383,243, filed Nov. 10, 2022, U.S. Provisional Application No. 63/385,948, filed Dec. 2, 2022, U.S. Provisional Application No. 63/483,936, filed Feb. 8, 2023, U.S. Provisional Application No. 63/486,946, filed Feb. 24, 2023, U.S. Provisional Application No. 63/491,526, filed Mar. 21, 2023, U.S. Provisional Application No. 63/497,691, filed Apr. 21, 2023, U.S. Provisional Application No. 63/502,611, filed May 16, 2023, U.S. Provisional Application No. 63/505,053, filed May 30, 2023, and U.S. Provisional Application No. 63/581,609, filed Sep. 8, 2023, all entitled “METHODS AND USES OF TACI-FC FUSION IMMUNOMODULATORY PROTEIN” the contents of which are incorporated by reference in their entirety.

### REFERENCE TO AN ELECTRONIC SEQUENCE LISTING

The contents of the electronic sequence listing (761612004340SEQLIST.xml; Size: 345,811 bytes; and Date of Creation: Sep. 27, 2023) is herein incorporated by reference in its entirety.

### FIELD

The present disclosure provides methods of treatment and uses involving an immunomodulatory TACI-Fc fusion protein that exhibits neutralizing activity of BAFF and APRIL (or BAFF/APRIL heterotrimers). The provided TACI-Fc fusion protein may include variant domains of Transmembrane Activator and CAML Interactor (TACI). The methods and uses provide therapeutic utility for a variety of immunological diseases, disorders or conditions, such as B cell-mediated diseases, disorder or conditions.

### BACKGROUND

Modulation of the immune response by intervening in processes involving interactions between soluble ligands and their receptors is of increasing medical interest. Currently, biologics used to enhance or suppress immune responses have generally been limited to antibodies (e.g., anti-PD-1 antibodies) or soluble receptors against a single cell surface molecule (e.g., CTLA-4-Fc). Improved therapeutic agents that can modulate the immune response, and particularly B cell immune responses, are needed. Provided are embodiments that meet such needs.

### SUMMARY

In some aspects, provided herein is a method of treating an autoantibody-related disease or disorder in a subject, the method comprising administering to the subject a TACI-Fc fusion protein that is a homodimer of two polypeptides of the formula TACI-linker-Fc, wherein TACI is a variant TACI polypeptide comprising one or more amino acid substitutions selected from the group consisting of K77E, F78Y and Y102D in the amino acid sequence set forth in SEQ ID NO:13, and wherein the TACI-Fc fusion protein is administered subcutaneously at a dose of from at or about 80 mg to at or about 480 mg once every four weeks (Q4W). In some aspects, provided herein is a method of treating an autoantibody-related disease or disorder in a subject, the method comprising administering to the subject a TACI-Fc fusion protein that is a homodimer of two polypeptides of the formula TACI-linker-Fc, wherein TACI is a variant TACI polypeptide comprising one or more amino acid substitutions selected from the group consisting of K77E, F78Y and Y102D in the amino acid sequence set forth in SEQ ID NO:13, and wherein the TACI-Fc fusion protein is administered subcutaneously at a dose of from at or about 24 mg to at or about 480 mg once every four weeks (Q4W), once every eight weeks (Q8W), or once every twelve weeks (Q12W).

In some embodiments, the variant TACI polypeptide comprises the amino acid substitutions K77E, F78Y and Y102D.

In some embodiments, the dose is from at or about 80 mg to at or about 240 mg Q4W. In some embodiments, the dose is at or about 80 mg Q4W. In some embodiments, the dose is at or about 240 mg Q4W. In some embodiments, the dose is from at or about 24 mg to at or about 240 mg once every four weeks (Q4W), once every eight weeks (Q8W), or once every twelve weeks (Q12W). In some embodiments, the dose is at or about 24 mg Q4W. In some embodiments, the dose is at or about 24 mg Q8W. In some embodiments, the dose is at or about 24 mg Q12W. In some embodiments, the dose is at or about 80 mg Q8W. In some embodiments, the dose is at or about 80 mg Q12W. In some embodiments, the dose is at or about 240 mg Q8W. In some embodiments, the dose is at or about 240 mg Q12W.

In some embodiments, the autoantibody-related disease or disorder is selected from the group consisting of a rheumatic disease or disorder, a renal (kidney) disease or disorder, a hematologic disease or disorder, a dermatologic disease or disorder, or a neurologic disease or disorder. In some embodiments, the autoantibody-related disease or disorder is a rheumatic disease or disorder. In some embodiments, the autoantibody-related disease or disorder is Sjogren's. In some embodiments, the autoantibody-related disease or disorder is Systemic lupus erythematosus (SLE).

In some embodiments, the TACI-Fc fusion protein reduces the risk of the subject developing hypogammaglobulinemia or severe hypogammaglobulinemia. In some embodiments, hypogammaglobulinemia is characterized by circulating IgG≤7 g/L. In some embodiments, severe hypogammaglobulinemia is characterized by circulating IgG<3 g/L. In some embodiments, severe hypogammaglobulinemia is characterized by circulating IgG<1.5 g/L. In some embodiments, severe hypogammaglobulinemia is characterized by circulating IgG<1.0 g/L. In some embodiments, the TACI-Fc fusion protein reduces the amount of circulating immunoglobulin G (IgG). In some embodiments, circulating IgG is reduced by about 35% from the subject's baseline.

In some aspects, provided herein is a method of treating Systemic lupus erythematosus (SLE), the method comprising: a) selecting a subject for administration of a TACI-Fc fusion protein that has been diagnosed with SLE; and b) administering to the selected subject the TACI-Fc fusion protein, wherein: the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and the TACI-Fc fusion protein is administered subcutaneously at a dose of from at or about 80 mg to at or about 480 mg once every four weeks.

In some embodiments, the dose is from at or about 80 mg to at or about 240 mg Q4W. In some embodiments, the dose is at or about 80 mg Q4W. In some embodiments, the dose is at or about 240 mg Q4W.

In some embodiments, the systemic lupus erythematosus is mild to moderate systemic lupus erythematosus or moderate to severe systemic lupus erythematosus. In some embodiments, the systemic lupus erythematosus is mild systemic lupus erythematosus. In some embodiments, the systemic lupus erythematosus is moderate systemic lupus erythematosus. In some embodiments, the systemic lupus erythematosus is severe systemic lupus erythematosus.

In some embodiments, the subject is selected for treatment if at the time of screening the subject has active SLE for ≥6 months.

In some embodiments, the subject is selected for treatment if at the time of screening the SLE is characterized by one or more of the following: (i) a hybrid SELENA-SLEDAI score ≥8 or a hybrid SELENA-SLEDAI ≥6 if there is high anti-dsDNA or low complement (C) levels; (ii) ≤6 g/g urine total protein to creatinine ratio (proteinuria); (iii) A grade in the BILAG score in ≥1 organs; (iv) B grade in the BILAG score in ≥2 organs; and (v) Physicians Global Assessment (PGA) score ≥1.0.

In some embodiments, the subject is receiving standard therapy for treating the SLE.

In some embodiments, the subject is selected for treatment if the at the time of screening or the time of administering the TACI-Fc fusion protein, the subject is receiving a stable standard treatment regimen characterized by the stable use of a standard therapy for treating the SLE. In some embodiments, the stable use is stable use of the standard therapy for at least 30 days.

In some embodiments, the TACI-Fc fusion protein is administered to the subject in combination with a standard therapy for treating the SLE.

In some embodiments, the standard therapy comprises one of more of a corticosteroid, antimalarial (e.g. hydroxychloroquine), an non-steroidal anti-inflammatory drug (NSAID), or an immunosuppressant or immunomodulator, or any combination thereof.

In some embodiments, the immunosuppressant or immunomodulator is selected from the group consisting of including azathioprine, mycophenolate (e.g. mycophenolate mofetil or sodium mycophenolate), cyclophosphamide, methotrexate, leflunomide, tacrolimus, cyclosporine and combinations of any of the foregoing.

In some embodiments, the standard therapy comprises a corticosteroid and administration of the corticosteroid is tapered after administering the TACI-Fc fusion protein.

In some embodiments, the subject is selected for treatment if at the time of screening the subject is characterized by one or more of the following: (i) severe lupus nephritis, such as defined as urine protein >6 g/24 hours or serum creatinine>2.5 mg/dL or 221 μmol/L; (ii) required hemodialysis; (iii) received high-dose corticosteroids for ≥14 days in the last 2 months, for example in which the high-dose corticosteroid is treatment with prednisone>100 mg/day or equivalent; and (iv) central nervous system disease caused by SLE or not caused by SLE in the last 2 months. In some aspects, the central nervous system disease is epilepsy, psychosis, organic brain syndrome, cerebrovascular accident, encephalitis, or central nervous system vasculitis.

In some embodiments, the autoantibody-related disease or disorder is a renal (kidney) disease or disorder. In some embodiments, the autoantibody-related disease or disorder is a Glomerulonephritis. In some aspects, provided herein is a method of treating a Glomerulonephritis, the method comprising: a) selecting a subject for administration of a TACI-Fc fusion protein that has been diagnosed with glomerulonephritis; and b) administering to the selected subject the TACI-Fc fusion protein, wherein: the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and the TACI-Fc fusion protein is administered subcutaneously at a dose of from at or about 80 mg to at or about 480 mg once every four weeks.

In some embodiments, the dose is from at or about 80 mg to at or about 240 mg Q4W. In some embodiments, the dose is at or about 80 mg Q4W. In some embodiments, the dose is at or about 240 mg Q4W.

In some embodiments, the subject is selected for treatment if at the time of screening the subject has active Glomerulonephritis. In some embodiments, the Glomerulonephritis is selected from the group consisting of IgA Nephropathy, Lupus Nephritis and Primary Membranous Nephropathy.

In some embodiments, the Glomerulonephritis is IgA Nephropathy and the subject is selected for treatment if at the time of screening the subject is characterized by one or both of the following: (i) the subject was diagnosed with IgA Nephropathy ≤5 years prior to the screening; and (ii) ≥0.75 g/g urine total protein to creatinine ratio (proteinuria). In some embodiments, the Glomerulonephritis is IgA Nephropathy and the subject is selected for treatment if at the time of screening the subject is characterized by one or more of the following: (i) the subject was diagnosed with IgA Nephropathy ≤5 years prior to the screening; (ii) ≥0.75 g/g urine total protein to creatinine (proteinuria); and (iii) elevated galactose deficient IgAQ1 (Gd-IgA1). In some embodiments, the TACI-Fc fusion protein reduces Gd-IgAQ1. In some embodiments, Gd-IgA1 is reduced by more than 50%.

In some embodiments, the Glomerulonephritis is Lupus Nephritis and the Lupus Nephritis is Class III (active focal), Class IV (diffuse) or Class V (lupus membranous nephropathy).

In some embodiments, the Glomerulonephritis is Lupus Nephritis and the subject is selected for treatment if at the time of screening the subject is characterized by one or more of the following: (i) the subject was diagnosed with Lupus Nephritis Class II-V≤3 years prior to the screening; (ii) ≥1 g/g urine total protein to creatinine ratio (proteinuria); (iii) active urinary sediment; (iv) positive anti-dsDNA and antinuclear antibodies (ANA), such as wherein positive anti-dsDNA is a titer of ≥30 IU/mL and positive ANA is a titer of ≥1:80; and (v) stable standard treatment regimen characterized by the stable use of a standard therapy for treating the SLE, such as wherein the stable use is stable use of the standard therapy for at least 30 days; and (v) received stable background immunosuppression, such as wherein the stable background immunosuppression is a stable dose of MMF of 1 g/day, with or without corticosteroids, for at least 8 weeks prior to the time of screening or the time of administering the TACI-Fc fusion protein.

In some embodiments, the Glomerulonephritis is primary Membranous Nephropathy.

In some embodiments, the Glomerulonephritis is primary Membranous Nephropathy (pMN) and the subject is selected for treatment if at the time of screening the subject is characterized by one or more of the following: (i) the subject was diagnosed with pMN≤5 years prior to the screening; (ii) ≥3.5 g/g urine total protein to creatinine ratio (proteinuria); and (iii) positive anti-PLA2R1 or positive anti-THSD7A antibodies.

In some embodiments, the subject is selected for treatment if at the time of screening or the time of administering the TACI-Fc fusion protein the subject has received therapy with an Angiotensin-converting enzyme (ACE) inhibitor and/or angiotensin II receptor blocker (ARB), such as wherein the subject has received a maximally recommended dose of the ACE inhibitor or ARB therapy.

In some embodiments, the subject is selected for treatment if at the time of screening or at the time of administering the TAC-Fc fusion protein the subject has a stable blood pressure.

In some embodiments, the autoantibody-related disease or disorder is a hematological disease or disorder. In some embodiments, the autoantibody-related disease or disorder is an autoimmune cytopenia.

In some aspects, provided herein is a method of treating an autoimmune cytopenia, the method comprising: a) selecting a subject for administration of a TACI-Fc fusion protein that has been diagnosed with an autoimmune cytopenia; and b) administering to the selected subject the TACI-Fc fusion protein, wherein: the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and the TACI-Fc fusion protein is administered subcutaneously at a dose of from at or about 80 mg to at or about 480 mg once every four weeks.

In some embodiments, dose is from at or about 80 mg to at or about 240 mg Q4W. In some embodiments, the dose is at or about 80 mg Q4W. In some embodiments, the dose is at or about 240 mg Q4W.

In some embodiments, the subject is selected for treatment if at the time of screening the subject has active cytopenia.

In some embodiments, the autoimmune cytopenia is selected from the group consisting of Immune Thrombocytopenia (ITP) and Autoimmune Hemolytic Anemia (AIHA).

In some embodiments, the autoimmune cytopenia is ITP and the subject is selected for treatment if at the time of screening the subject is characterized by one or more of the following: (i) the subject was diagnosed with ITP≥3 months prior to the screening; (ii) sustained platelet count <30,000/μL; and (iii) received ≥4 prior treatments for treating the ITP.

In some embodiments, the autoimmune cytopenia is an AIHA and the AIHA is warm AIHA (wAIHA) or cold AIHA (cold agglutinin disease, CAD).

In some embodiments, the autoimmune cytopenia is wAIHA or CAD and the subject is selected for treatment if at the time of screening the subject is characterized by one or more of the following: (i) the subject was diagnosed with wAIHA or CAD≥3 months prior to the screening; (ii) sustained hemoglobin (Hb)<9 g/dL; and (iii) received ≥2 prior treatments for treating the AIHA.

In some embodiments, the autoimmune cytopenia is wAIHA. In some embodiments, the autoimmune cytopenia is CAD.

In some embodiments, the subject is selected for treatment if at the time of screening or the time of administering the TACI-Fc fusion protein, the subject is receiving a stable immunosuppression.

In some embodiments, the TACI-Fc fusion protein is administered to the subject in combination with concurrent administration of the stable immunosuppression.

In some embodiments, the stable immunosuppression comprises a stable dose of a steroid, such as a corticosteroid, for at least two weeks prior to the time of screening or the time of administering the TACI-Fc fusion protein; and/or the stable immunosuppression comprises a stable dose of azathioprine, MMF, or cyclosporine for at least four weeks prior to the time of screening or the time of administering the TACI-Fc fusion protein.

In some embodiments, the subject is not characterized by having a secondary cytopenia (e.g. systemic autoimmune disease or malignancy) or Evans syndrome.

In some embodiments, the autoantibody-related disease or disorder is a dermatologic disease or disorder. In some embodiments, the autoantibody-related disease or disorder is an autoimmune bullous dermatosis.

In some aspects, provided herein is a method of treating an autoimmune bullous (blistering) dermatosis, the method comprising: a) selecting a subject for administration of a TACI-Fc fusion protein that has been diagnosed with an autoimmune bullous (blistering) dermatosis; and b) administering to the selected subject the TACI-Fc fusion protein, wherein: the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and the TACI-Fc fusion protein is administered subcutaneously at a dose of from at or about 80 mg to at or about 480 mg once every four weeks.

In some embodiments, the dose is from at or about 80 mg to at or about 240 mg Q4W. In some embodiments, the dose is at or about 80 mg Q4W. In some embodiments, the dose is at or about 240 mg Q4W.

In some embodiments, the subject is selected for treatment if at the time of screening the subject has active blistering disease. In some embodiments, the autoimmune bullous (blistering) dermatosis is selected from the group consisting of Pemphigus vulgaris, Pemphigus foliaceus or Bullous Pemphigoid.

In some embodiments, the autoimmune bullous (blistering) dermatosis is Pemphigus vulgaris or Pemphigus foliaceus and the subject is selected for treatment if at the time of screening the subject is characterized by one or both of the following: (i) a Pemphigus Disease Area Index (PDAI)≥15; and (ii) positive anti-Dsg1 or positive anti-Dsg3 antibodies.

In some embodiments, the autoimmune bullous (blistering) dermatosis is Pemphigus vulgaris. In some embodiments, the autoimmune bullous (blistering) dermatosis is Pemphigus foliaceus.

In some embodiments, the autoimmune bullous (blistering) dermatosis is Pemphigoid and the subject is selected for treatment if at the time of screening the subject is characterized by one or both of the following: (i) IgA antibodies; and (ii) positive anti-Bp180 or positive anti-Bp230 antibodies.

In some embodiments, the subject is selected for treatment if at the time of screening or the time of administering the TACI-Fc fusion protein, the subject is receiving a stable immunosuppression.

In some embodiments, the TACI-Fc fusion protein is administered to the subject in combination with concurrent administration of the stable immunosuppression.

In some embodiments, the stable immunosuppression comprises a stable dose of a steroid, such as a corticosteroid, for at least two weeks prior to the time of screening or the time of administering the TACI-Fc fusion protein; and/or the stable immunosuppression comprises a stable dose of azathioprine, MMF, or cyclosporine for at least four weeks prior to the time of screening or the time of administering the TACI-Fc fusion protein.

In some embodiments, the subject is not characterized by having a secondary disease (e.g. paraneoplastic).

In some embodiments, the autoantibody-related disease or disorder is a neurologic disease or disorder. In some embodiments, the autoantibody-related disease or disorder is Encephalitis.

In some aspects, provided herein is a method of treating Encephalitis, the method comprising: a) selecting a subject for administration of a TACI-Fc fusion protein that has been diagnosed with Encephalitis; and b) administering to the selected subject the TACI-Fc fusion protein, wherein: the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and the TACI-Fc fusion protein is administered subcutaneously at a dose of from at or about 80 mg to at or about 480 mg once every four weeks.

In some embodiments, the dose is from at or about 80 mg to at or about 240 mg Q4W. In some embodiments, the dose is at or about 80 mg Q4W. In some embodiments, the dose is at or about 240 mg Q4W.

In some embodiments, the Encephalitis is autoimmune encephalitis. In some embodiments, the Encephalitis is Limbic encephalitis.

In some embodiments, the TACI-Fc fusion protein is administered to the subject Q4W for between 12 weeks and 72 weeks. In some embodiments, the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more. In some embodiments, the TACI-Fc fusion protein is administered to the subject Q4W for 12 weeks. In some embodiments, the TACI-Fc fusion protein is administered to the subject Q4W for 16 weeks. In some embodiments, the TACI-Fc fusion protein is administered to the subject Q4W for 24 weeks. In some embodiments, the TACI-Fc fusion protein is administered to the subject Q4W for 48 weeks.

In some embodiments of any of the provided methods, instead of administering the TACI-Fc fusion protein to the subject Q4W, alternative embodiments contemplate administering the TACI-Fc fusion protein to the subject Q8W or Q12W. In some of any such embodiments of any of the provided methods, the subject is administered the TACI-Fc fusion protein in a dose that is from at or about 240 mg to at or about 480 mg Q8W, such as at or about 240 mg Q8W, at or about 320 mg Q8W, or at or about 480 mg Q8W. In some of any such embodiments of any of the provided methods, the subject is administered the TACI-Fc fusion protein in a dose that is from at or about 240 mg to at or about 480 mg Q12W, such as at or about 240 mg Q12W, at or about 320 mg Q12W, or at or about 480 mg Q12W.

In some embodiments, the variant TACI polypeptide is set forth in SEQ ID NO:26.

In some embodiments, the linker is a GS linker of between 5 and 20 amino acids in length. In some embodiments, the linker is selected from GSGGS (SEQ ID NO: 76), GGGGS (G4S; SEQ ID NO: 77), GSGGGGS (SEQ ID NO: 74), GGGGSGGGGS (2×GGGGS; SEQ ID NO: 78), GGGGSGGGGSGGGGS (3×GGGGS; SEQ ID NO: 79), GGGGSGGGGSGGGGSGGGGS (4×GGGGS, SEQ ID NO:84), GGGGSGGGGSGGGGSGGGGSGGGGS (5×GGGGS, SEQ ID NO: 91), GGGGSSA (SEQ ID NO: 80), or GSGGGGSGGGGS (SEQ ID NO:194) or combinations thereof. In some embodiments, the linker is set forth in SEQ ID NO: 74.

In some embodiments, the Fc is an IgG1 Fc domain. In some embodiments, the Fc is a variant IgG1 Fc that exhibits reduced binding affinity to an Fc receptor and/or reduced effector function as compared to a wild-type IgG1 Fc domain.

In some embodiments, the variant IgG1 Fc domain comprises one or more amino acid substitutions selected from L234A, L234V, L235A, L235E, G237A, S267K, R292C, N297G, and V302C, by EU numbering. In some embodiments, the variant IgG1 Fc comprises the amino acid substitutions L234A, L235E, and G237A by EU numbering.

In some embodiments, the Fc comprises the amino acid substitution C220S, wherein the residues are numbered according to the EU index of Kabat.

In some embodiments, the Fc lacks the hinge sequence EPKSS or EPKSC.

In some embodiments, the Fc region comprises K447del, wherein the residue is numbered according to the EU index of Kabat. In some embodiments, the Fc comprises the amino acid sequence set forth in SEQ ID NO:73.

In some embodiments, the TACI-Fc fusion protein is set forth in SEQ ID NO: 167.

In some embodiments, the Fc comprises the amino acid sequence set forth in SEQ ID NO:81.

In some embodiments, the TACI-Fc fusion protein is set forth in SEQ ID NO: 168.

In some embodiments, the TACI-Fc fusion protein is provided in a formulation comprising an acetic acid buffer having a pH of from about 4.0 to about 6.0, proline at a concentration of from at or about 1% to about 10%, and a surfactant at a concentration of from about 0.005 to about 0.05% (w/v).

In some embodiments, the formulation has a pH of about 5.2.

In some embodiments, the acetic acid buffer comprises a concentration of acetate of from at or about 5 mM to at or about 15 mM. In some embodiments, the acetic acid buffer comprises a concentration of acetate of at or about 10 mM.

In some embodiments, the proline is at a concentration of about 2% to about 5%. In some embodiments, the proline is at a concentration of at or about 3%.

In some embodiments, the surfactant is at a concentration of from about 0.01 to about 0.025% (w/v), such as at or about 0.015% (w/v). In some embodiments, the surfactant is polysorbate 80.

In some embodiments, the amount of TACI-Fc fusion protein in the formulation is from about 50 mg to about 100 mg. In some embodiments, the amount of TACI-Fc fusion protein in the formulation is at or about 80 mg.

In some embodiments, the concentration of the TACI-Fc fusion protein is between about 50 mg/mL and about 200 mg/mL. In some embodiments, the concentration of the TACI-Fc fusion protein is at or about 100 mg/mL.

In some embodiments, a B cell immune response or activity is reduced in the subject. In some embodiments, the numbers of mature and total circulating B cells are reduced in the subject.

In some embodiments, circulating serum immunoglobulins are reduced in the subject.

In some embodiments, one or more of B cell maturation, differentiation, and/or proliferation is reduced or inhibited.

In some embodiments, circulating levels of an APRIL or BAFF protein are reduced in the subject. In some embodiments, the APRIL or BAFF protein is an APRIL homotrimer, BAFF homotrimer, APRIL/BAFF heterotrimer, or BAFF 60mer.

In some embodiments, the subject is a human.

In some embodiments, the subject is an adult subject. In some embodiments, the subject is 18 years of age or older, such as 18-65 years of age.

### BRIEF DESCRIPTION OF THE DRAWINGS

FIG. 1 shows a schematic representation of a functional inhibition assay involving recombinant APRIL and BAFF by TAC. In the assay, Jurkat cells transduced with a luciferase-based NF-κB reporter and to stably express mouse or human TACI on the cell-surface expression. Following activation by recombinant APRIL or BAFF, endogenous NF-κB transcription factors bind to the DNA response elements controlling transcription of a firefly luciferase gene. Luciferase expression can be monitored, such as by detection with Bio-Glo™ reagent and measurement using a Cytation 3 reader.

FIG. 2 shows exemplary human TACI TD Fc fusion molecules for blockade of human APRIL (top panel) and BAFF (bottom panel) mediated signaling. TACI TD Fc fusions were incubated with APRIL or BAFF for 20 mins (room temperature with shaking) and then added to wells containing 150,000 Jurkat/TACI/NFκB-luciferase cells for 5 hours.

FIG. 3A shows function of exemplary TACI TD Fc fusion molecules for blockade of APRIL (top panel of the FIG) or BAFF (bottom panel of the FIG).

FIG. 3B shows human TACI TD Fc fusion molecules for blockade of mouse APRIL (left panel) and BAFF (right panel) mediated signaling.

FIG. 4A shows human TACI TD Fc fusion molecules for blockade of human APRIL (tope panel) and BAFF (bottom panel) mediated signaling relative to TACI 13-118-Fc, TACI 30-110-Fc, and belimumab.

FIGS. 4B-4C depict assessment of APRIL and BAFF inhibitory activity and binding by various TACI Fc-fusion proteins and affinity optimized TACI variant 26 TACI CRD2-Fc. FIG. 4B shows APRIL and BAFF inhibition by the indicated TACI variants and 26 TACI CRD2-Fc evaluated in the TACI/Jurkat/NF-κB reporter assay. Increased inhibitory activity is indicated by reduced luciferase production. FIG. 4C shows SPR sensorgrams with 26 TACI CRD2-Fc and telitacicept at medium density shown in black lines and results from non-linear least squares regression analysis of the data shown in orange lines. Telitacicept was sourced through Clinigen.

FIG. 5A-C shows exemplary human TACI TD Fc fusion molecule 26 TACI CRD2-Fc for blockade of BAFF- (FIG. 5A), APRIL- (FIG. 5B), and a combination of BAFF+APRIL-mediated (FIG. 5C) signaling relative to belimumab, BION-1301, and WT TACI-Fc molecules including WT TACI 30-110 (atacicept) and WT TACI 13-118-Fc (telitacicept).

FIGS. 5D-5F depict that 26 TACI CRD2-Fc inhibits APRIL and BAFF more potently than comparator molecules. FIGS. 5D-5F show APRIL, BAFF, or APRIL plus BAFF inhibition by 26 TACI CRD2-Fc and the indicated comparator molecules was evaluated in the TACI/Jurkat/NF-κB reporter assay.

FIGS. 5G-5J depict inhibition of BAFF multimers and BAFF/APRIL heterotrimers by 26 TACI CRD2-Fc in the TACI/Jurkat/NF-κB assay relative to comparator molecules. FIG. 5G shows BAFF 60-mer and FIGS. 5H-5J show heterotrimeric BAFF/APRIL and homotrimeric BAFF inhibition by 26 TACI CRD2-Fc, TACI 30-110-Fc, telitacicept, belimumab, and anti-APRIL mAb based on the VIS649 mAb sequence. Curve fit with GraphPad Prism log(agonist) vs. Response. Constraints: Hillslope=−1, F=50.

FIG. 5K shows exemplary human 26 TACI CRD2-Fc fusion molecule affinity for human BAFF (left panel) and APRIL (right panel) as determined by surface plasmon resonance (SPR) relative to WT TACI-Fc (Telitacicept).

FIGS. 6A-6L show analysis of parameters assessed in an NZB/NZW murine model of human SLE. Proteinuria scores (FIG. 6A), mean percent change in body weight (FIG. 6B), and percent survival (FIG. 6C) were assessed starting at 20 weeks of age. Serum was analyzed for anti-double stranded DNA IgG titers (FIG. 6D) and blood urea nitrogen (BUN) (FIG. 6E and FIG. 6L) (**** vs Fc by Student's t-test, p<0.0001 for anti-dsDNA IgG; *** vs Fc by Student's t-test, p=0.0008 for BUN). Kidneys were processed and analyzed by histology in replicate Periodic acid-Schiff (PAS)-stained sections, with individual component and total histology scores depicted in FIG. 6F. Frozen kidneys were also sectioned and stained for immunohistochemical analysis of mouse IgG and complement C3 glomerular deposition, as shown in FIG. 6G and FIG. 6H, respectively. FIG. 6I shows the histological score ±SEM. Sialadentis as measured by submandibular gland histology score is shown in FIG. 6J. FIG. 6K shows renal IgG deposit score (mean±SD) evaluated by IHC from right kidney at termination (FIG. 6K, left) and a representative IHC (10×) of renal IgG deposits from Fc control or 26 TACI CRD2-Fc-treated mouse (FIG. 6K, left).

FIG. 7 shows the ability of TACI mutations (K77E/F78Y/Y102D) to inhibit APRIL (left panel) and BAFF (right panel) mediated signaling, quantified by luciferase production in Jurkat/NF-κB/TACI cells.

FIG. 8A and FIG. 8B depict schematic representations of exemplary TACI-Fc fusion proteins. FIG. 8A depicts an exemplary TACI-Fc fusion protein containing two cysteine-rich pseudo-repeats (CRD). FIG. 8B depicts an exemplary TACI-Fc fusion protein containing one cysteine-rich pseudo-repeat (CRD, e.g. CRD2).

FIG. 9 depicts exemplary sequence alignments to identify corresponding residues in a sequence compared to a reference sequence. The symbol “*” between two aligned amino acid indicates that the aligned amino acids are identical. The symbol “-” indicates a gap in the alignment. Exemplary, non-limiting positions for amino acid substitution described herein are indicated with bold text. Based on the alignment of two similar sequences having identical residues in common, a skilled artisan can identify “corresponding” positions in a sequence by comparison to a reference sequence using conserved and identical amino acid residues as guides. FIG. 9 provides an exemplary alignment of a reference TACI extracellular domain sequence set forth in SEQ ID NO:122 (containing the full extracellular domain with a CRD1 and CRD2 and an initiating methionine residue) with a TACI extracellular domain sequence set forth in SEQ ID NO:13 (containing only a single CRD, CRD2); aligning identical residues demonstrates, for example, that amino acid residue E7 in SEQ ID NO:13 corresponds to residue E74 in SEQ ID NO: 122, amino acid residue K10 in SEQ ID NO: 13 corresponds to residue K77 in SEQ ID NO:122, amino acid residue Y12 in SEQ ID NO: 13 corresponds to Y79 in SEQ ID NO:122, amino acid residue L15 in SEQ ID NO:13 corresponds to L82 in SEQ ID NO:122, amino acid residue R17 in SEQ ID NO: 13 corresponds to R84 in SEQ ID NO:122; and amino acid residue D16 in SEQ ID NO:13 correspond to D85 in SEQ ID NO:122. It is within the level of a skilled artisan to carry out similar alignments between two similar protein sequences to identify corresponding residues, including based on the exemplification and description herein.

FIGS. 10A-10D show analysis of parameters assessed murine keyhole limpet hemocyanin (KLH) model. Serum-KLH IgM OD levels were assessed as primary response (FIG. 10A) and secondary response (FIG. 10B). Similarly, serum anti-KLH IgG1 OD levels were assessed as both primary response (FIG. 10C) and secondary response (FIG. 10D).

FIGS. 11A-11B show analysis of harvested spleen assessed from the murine keyhole limpet hemocyanin (KLH) immunization model. Spleens were processed and analyzed by weight (FIG. 11A) as well as total cell number (FIG. 11B).

FIG. 11C shows that 26 TACI CRD2-Fc affects splenocytes more potently than WT TACI-Fc in KLH-immunized mice. Total numbers of splenocytes were enumerated by flow cytometry.

FIG. 12A depicts analysis of spleens assessed for cellular subtype population. FIG. 12A shows splenic makeup from the murine keyhole limpet hemocyanin (KLH) model and shows results of B cell subset numbers relative to the group mean. FIGS. 12B-12C depict analysis of spleens assessed for cellular subtype phenotype makeup from the murine keyhole limpet hemocyanin (KLH) model and shows results for numbers of germinal center B cells and plasma cells (FIG. 12B). FIG. 12C shows splenic plasma cells with individual mice plotted.

FIGS. 12D-12J depict that 26 TACI CRD2-Fc affects splenic B and T cell subsets more potently than WT TACI-Fc in KLH-immunized mice. FIGS. 12D-12J show total numbers of indicated splenic B cell subsets (i.e., T1 B cell, B cell, T2 B cell, GC cell, FOL B cell, MZ B cell and plasma cell) on Day 20, which were enumerated by flow cytometry.

FIG. 13A depicts a gating scheme for quantifying B cell subsets and plasma cells in mouse spleens. Cells were gated away from debris in the FSC-A/SSC-A dot plot. This gate was analyzed by FSC-H/FSC-A and then SSC-H/SSC-W dot plots to gate cells along established diagonals that exclude doublet cell populations. The CD45+/LiveDead Aqua viability-negative cells were gated from the SSC-H/SSC-W singlet gate to identify live CD45+ cells. The live CD45±cell gate was then analyzed by a B220/Gr1 dot plot. The B220+/Gr1− cells were then analyzed by a GL7/CD95 dot plot to identify GL7+/CD95+GC B cells. B220+/Gr1− cells were also analyzed by a CD138/CD19 dot plot to identify CD19+ cells, which were subsequently analyzed by a CD23/CD19 dot plot. The CD23+/CD19+ cells were further analyzed by CD21/IgM expression to identify CD21+/IgM+ Follicular (FOL) B cells and CD21^{br}/IgM^{br} T2 B cells. The CD23−/CD19+ cells were also analyzed by a CD21/IgM dot plot to identify CD21-/IgM^{br} transitional type-1 (T1) B cells and CD21^{br}/IgM^{br} MZ B cells. The live CD45+ cell gate was also analyzed by a B220/CD19 dot plot to identify B220^{+/lo}/CD19+ B cells. The B220^{lo}/CD138+ plasma cells were gated from the B220^{+/lo}/CD19+ gate. FSC-A=forward scatter area; SSC-A=side scatter area; H=height; W=width.

FIG. 13B depicts gating scheme for quantifying CD4+T_{FH} cells in mouse spleens. Cells were gated away from debris in the FSC-A/SSC-A dot plot. This gate was analyzed by FSC-H/FSC-A and then SSC-H/SSC-W dot plots to gate cells along established diagonals that exclude doublet cell populations. The CD45⁺/LiveDead Aqua viability-negative cells were gated from the SSC-H/SSC-W singlet gate to identify live CD45⁺ cells. The live CD45⁺ cell gate was then analyzed by a B220/CD3 dot plot. The CD3+ T cells were then analyzed by a CD4/CD8 dot plot. CD4⁺ T cells were analyzed by a PD1/CXCR5 dot plot to identify PD1+CXCR5+T_{FH}cells. T_{FH}=T follicular help; FSC-A=forward scatter area; SSC-A=side scatter area; H=height; W=width.

FIGS. 14A-D depict T cell numbers in the murine keyhole limpet hemocyanin (KLH) model. The splenic CD3+, CD8+, CD4+ and Follicular Helper T cells are depicted in FIG. 14A, FIG. 14B, FIG. 14C, and FIG. 14D, respectively.

FIGS. 14E-14F show splenic T cells were also enumerated by flow cytometry. Individual mice are plotted, and the mean±SD shown as horizontal line and error bars, respectively. GC=germinal center; T1=transitional-1 B cell; T2=transitional-2 B cell; FOL=follicular; MZ=marginal zone.

FIG. 14G shows total number of T_{FH} cells/spleen from KLH-challenged or naïve mice at Day 20, enumerated by flow cytometry, with individual mice plotted.

FIG. 15 depicts Tcm and Tem cellular populations in the murine keyhole limpet hemocyanin (KLH) model.

FIGS. 16A-16B and FIGS. 17A-17B depict overall incidence and degree of sialadenitis (FIGS. 16A-16B) and insulitis (FIGS. 17A-17B) in diabetes-prone mice after treatment with the tested molecules.

FIG. 18 and FIG. 19 depict serum immunoglobulin (IgM, IgA, and IgG) concentrations for exemplary tested molecules in a pharmacokinetic/pharmacodynamic study following a single intravenous infusion in male Sprague Dawley rats.

FIG. 20A and FIG. 20B depict individual animal serum concentrations versus time profiles for exemplary tested molecules administered to cynomolgus monkeys in a PK/PD model. The results depicted in FIG. 20B for Atacicept are based on published data (Carbonatto et al. (2008) Toxicol Sci 105:200-210).

FIGS. 21A-21B depict the levels of serum IgM, IgA, and IgG in animals receiving exemplary tested molecules. FIG. 21A shows IgM, IgA and IgG PK/PD in a cynomolgus monkey PK/PD model. FIG. 21B shows levels of serum IgM, IgA, and IgG (mean+range) in each treatment group measured by ELISA at various timepoints and plotted as a percentage of baseline serum concentrations obtained from serum collected on Day −8.

FIG. 22 depicts absolute cell counts for animals receiving exemplary tested molecules in a cynomolgus monkey PK/PD model.

FIG. 23 depicts % of cells from baseline for animals receiving exemplary tested molecules in a cynomolgus monkey PK/PD model.

FIG. 24 depicts absolute counts or relative percentages of the proliferating T cells animals receiving exemplary tested molecules in a cynomolgus monkey PK/PD model.

FIGS. 25A-25B depict the predicted human PK profiles after repeated IV dosing every four weeks (FIG. 25A) or every two weeks (FIG. 25B) in a two-compartment PK model.

FIGS. 26A-26E depict inhibition of class-switched memory B cells (FIG. 26A), plasma cells (FIG. 26B) and immunoglobulin secretion (FIGS. 26C-26E).

FIGS. 26F and 26G show CD19+ B cells were activated with rhCD40L and re-cultured with exogenous APRIL, BAFF, and 26 TACI CRD2-Fc or the indicated comparator molecules. Cells were stained and analyzed by flow cytometry to identify class switched memory B cells (IgD, IgM, CD27+) or plasma cells (IgM, IgD, CD38+, CD319+). After 7 days, supernatants were collected and IgM (FIG. 26H), IgA (FIG. 26I), IgG1 (FIG. 26J), IgG2 (FIG. 26K), IgG3 (FIG. 26L), and IgG4 (FIG. 26M) secretion was quantitated by multiplex analysis. Statistically significant differences between group median values were determined using the Kruskal-Wallis test and uncorrected Dunn's test; p values <0.05 were considered statistically significant.

FIGS. 27A-27C depict the levels of plasma cells in the bone marrow (FIG. 27A), spleen (FIG. 27B) and lymph node (FIG. 27C) in CIA mouse models receiving the tested molecules.

FIG. 28 depicts the numbers of plasma cells in bone marrow smears of cynomolgus monkeys receiving the exemplary TACI-Fc fusion protein.

FIGS. 29A-29B depict dose-dependent serum concentrations versus time profiles (FIG. 29A) and % of cells from baseline (FIG. 29B) for animals receiving the exemplary TACI-Fc fusion protein in a cynomolgus monkey 1-month GLP toxicology study.

FIGS. 30A-30B depict levels of serum IgA, IgG, IgM, and IgE in animals receiving the exemplary TACI-Fc fusion in a cynomolgus monkey 1-month GLP toxicology study (FIG. 30A) and in a 6-month GLP toxicology study (FIG. 30B).

FIG. 31 analysis of harvested spleen assessed from the murine chronic Graft Versus Host Disease (cGVHD) model. Spleens were processed and analyzed by weight as well as total cell number.

FIG. 32 depicts analysis of spleens assessed for cellular population makeup from the murine chronic Graft Versus Host Disease model and shows results of CD45⁺ cell and B220⁺ B cell numbers.

FIG. 33 depicts analysis of spleens assessed for cellular subtype population makeup and shows results of CD4⁺ and CD8⁺ T cell subset numbers.

FIG. 34 depicts CD4⁺ T cell subset numbers in the cGVHD model.

FIG. 35A depicts B220⁺ B cells and CD1d^{hi}CD5+B-1 cell numbers in the cGVHD model. FIG. 35B depicts Transitional-1 (T1) and Tranisitional-2 (T2) B cell numbers in the cGVHD model.

FIG. 36A depicts follicular and marginal zone (MZ) B cell and FIG. 36B depicts germinal center (GC) B cells and plasma cell numbers in the cGVHD model.

FIG. 37 depicts early plasma cell, plasmablast, and long-lived plasma cell (LL-PC) numbers in the cGVHD model.

FIG. 38A depicts renal IgG immune complex deposits in the kidneys as measured by immunohistochemical staining with a fluorescently-labelled antibody specific for mouse IgG.

FIG. 38B shows a representative IHC (20×) of renal IgG deposits from Fc control, TACI CRD2-Fc (DAPI overlay in bottom right), or naïve mouse.

FIG. 39 shows analysis of anti-dsDNA autoantibody serum titers at weeks 8 and 13.

FIGS. 40A-40B show analysis of anti-dsDNA autoantibody serum titers in an H-2^{bm12} Mouse Model of Autoantibody-Related Glomerulonephritis across 56 days, i.e., 8 weeks (FIG. 40A) and at week 8 (FIG. 40B).

FIG. 41 depicts renal IgG immune complex deposits in the kidneys as measured by immunohistochemical staining with a fluorescently-labelled antibody specific for mouse IgG.

FIG. 42 depicts levels of serum IgA, IgM, and IgG (IgG1, IgG2b, and IgG3) in animals receiving the exemplary TACI-Fc fusion in a mouse model of Autoantibody-Related Glomerulonephritis.

FIGS. 43A-43B depict levels of anti-SRBC IgG1 (FIG. 43A) and plasma cells (FIG. 43B) in animals receiving 26 TACI CRD2-Fc compared to BAFF- and APRIL-specific biologics.

FIGS. 43C-43L depict that 26 TACI CRD2-Fc demonstrates enhanced immunosuppressive activity over telitacicept and BAFF- or APRIL-only inhibitors in a mouse SRBC immunization model. FIGS. 43C-43F show anti-SRBC Ig concentrations in serum measured on Day 15. The total number of germinal center (GC) B cells/spleen (FIG. 43G), CD4+T_{FH} cells/spleen (FIG. 43H), plasma cells (PC)/spleen (FIG. 43I), and plasmablasts (PB)/spleen (FIG. 43J) were enumerated by flow cytometry, with values for individual mice plotted. The percentage of long-lived plasma cells (LL-PC) (FIG. 43K) and total plasma cells (PC) (FIG. 43L) in the bone marrow were also determined by flow cytometry, with data for individual mice plotted. Data are presented as median±interquartile range (FIGS. 43G-43H) or mean±SD (FIGS. 43C-43F, 43I-43L).

FIGS. 44A-44D depict individual serum concentrations versus time profiles for 26 TACI CRD2-Fc fusion molecules in human cohorts administered 26 TACI CRD2-Fc via IV route or SC route. FIGS. 44A-44B depict 26 TACI CRD2-Fc serum concentrations across 56 days. FIGS. 44C-44D depict 26 TACI CRD2-Fc serum concentrations across 112 days.

FIGS. 45A-45B depict the serum IgA, IgG, IgM levels and their corresponding changes from baseline in human cohorts administered 26 TACI CRD2-Fc via IV route (FIG. 45A) or SC route (FIG. 45B).

FIGS. 45C-45F depict the serum galactose-deficient IgAQ1 (Gd-IgA1) levels and corresponding changes from baseline in human cohorts administered 26 TACI CRD2-Fc via IV route or SC route. FIGS. 45C-45D depict serum Gd-IgA1 levels across 28 days. FIGS. 45E-45F depict serum Gd-IgA1 levels across 112 days.

FIG. 46 depicts the serum IgA, IgG or IgM levels and their corresponding changes from baseline in human cohorts administered 80 mg 26 TACI CRD2-Fc SC as compared to the levels of comparators, Atacicept (first column from left), Telitacicept (second column), BION 1301 (third column) or Sibeprenlimab (fourth column), as determined from published data.

FIGS. 47A-47B depict dose-dependent, on-target reductions in the frequency of circulating CD19+CD38+CD27+IgD antibody secreting cells, including plasmablasts and plasma cells in human cohorts administered 26 TACI CRD2-Fc via IV route (FIG. 47A) or SC route (FIG. 47B).

FIGS. 47C-47D depict the frequency of circulating CD27-IgD+ antibody secreting cells, including Naïve B cells in human cohorts administered 26 TACI CRD2-Fc via IV route (FIG. 47C) or SC route (FIG. 47D).

FIGS. 47E-47F depict the frequency of circulating CD27+IgD− antibody secreting cells, including memory B cells in human cohorts administered 26 TACI CRD2-Fc via IV route (FIG. 47E) or SC route (FIG. 47F).

FIGS. 48A-48D show dose-dependent reductions, and durations thereof, in free APRIL (pg/mL or % change from baseline), in human cohorts administered 26 TACI CRD2-Fc via IV route (FIG. 48A, FIG. 48C) or SC route (FIG. 48B, FIG. 48D), which was observed through Day 28 post-dose or Day 56 post-dose.

FIGS. 48E-48F show dose-dependent reductions, and durations thereof, in free BAFF (% change from baseline), in human cohorts administered 26 TACI CRD2-Fc via IV route (FIG. 48E) or SC route (FIG. 48F), which was observed through Day 28 post-dose.

FIGS. 48G-48J show dose-dependent reductions, and durations thereof, in free APRIL (pg/mL) or free BAFF (pg/mL) in human cohorts administered 26 TACI CRD2-Fc via IV route (FIG. 48G, FIG. 48I) or SC route (FIG. 48H, FIG. 48J), which was observed through Day 112 post-dose.

FIGS. 49A-49G depict that 26 TACI CRD2-Fc demonstrates enhanced immunosuppressive activity over telitacicept and WT TACI CRD2-Fc in a mouse SRBC immunization model. FIGS. 49A-49D shows anti-SRBC Ig serum concentrations were measured on Day 15. FIG. 49E shows percent plasma cells in bone marrow enumerated by flow cytometry, with individual mice plotted. FIGS. 49E-49G show total number of germinal center (GC) B cells/spleen or CD4+T_{FH} cells/spleen, enumerated by flow cytometry, with individual mice plotted. Data are presented as median±interquartile range or mean±SD.

FIGS. 50A-50G depict that 26 TACI CRD2-Fc demonstrates enhanced immunosuppressive activity over telitacicept and anti-CD20 antibody in a mouse SRBC immunization model. FIGS. 50A-50D show anti-SRBC Ig serum levels measured on Day 15. FIG. 50E shows percentage of plasma cells in bone marrow enumerated by flow cytometry, with individual mice plotted. Bone marrow was not collected from the naïve group. FIGS. 50F-50G show total number of germinal center (GC) B cells/spleen or CD4+T_{FH} cells/spleen, enumerated by flow cytometry, with individual mice plotted. Data are presented as mean±SD.

FIGS. 51A-51C depict results following administration of 26 TACI CRD2-Fc in subjects with IgA nephropathy (IgAN) or primary membranous nephropathy (pMN). FIG. 51A shows urine protein: creatinine ratio and anti-SRBC Ig serum concentrations as measured on days 3, 8 and 15 post-administration of TACI CRD2-Fc. FIG. 51B shows urine protein: creatinine ratio and anti-SRBC Ig serum concentrations as measured on days 3, 8, 15, 22, 29, 43, and 57 post-administration of TACI CRD2-Fc. FIG. 51C shows urine protein: creatinine ratio as measured at 12 weeks post-administration of TACI CRD2-Fc in IgAN and pMN patients. FIG. 51D shows serum IgA, IgG, and IgM in IgAN patients.

FIGS. 51E-51J depict results following administration of 26 TACI CRD2-Fc in subjects with IgA nephropathy (IgAN). FIG. 51E depicts UPCR percent change from baseline (mean±SD) in a single (spot) UPCR test. FIG. 51F depicts UPCR percent change from baseline (mean±SD) in a 24-hour UPCR test. FIG. 51G depicts IgA, IgG, and IgM (mg/dL) as a percent change from baseline (mean±SD) in IgAN patients receiving 80 mg Q4W or 240 mg Q4W across time. FIG. 51H depicts Gd-IgAQ1 as a percent change from baseline (mean±SD) over time. FIG. 51I depicts eGFR as a change from baseline (mean±SD). FIG. 51J depicts eGFR as a percent change from baseline (mean±SD).

FIGS. 51K-51L depict results following administration of 26 TACI CRD2-Fc in a subject with primary membranous nephropathy (pMN). FIG. 51K depicts the percent change from baseline (mean±SD) in a 24-hour UPCR test. FIG. 51L depicts the change from baseline (mean±SD) of circulating levels of disease specific biomarker anti-phospholipase A2 Receptor (RU/mL).

FIGS. 52A-52G depict that 26 TACI CRD2-Fc provides benefit in an HEL-OVA-Duffy (HOD) mouse model of Autoimmune Hemolytic Anemia (AIHA). FIG. 52A shows an RBC-restricted triple fusion protein that can be bound by T cell receptor to initiate pathogenesis of AIHA. FIG. 52B shows HOD autoantibodies in HOD mice: that were not administered CTLA-4, IL-10R, LAG-3 or PD-1 antibodies (Pre-4Aby); 4 days before administering 26 TACI CRD2-Fc, an Fc control, or PBS (Day −4); and on days 9, 15, 23, and 28 post-administration of TACI CRD2-Fc, an Fc control, or PBS. FIG. 52C shows the change in HOD autoantibodies on Day 28 in HOD mice receiving 26 TACI CRD2-Fc, an Fc control, or PBS. FIG. 52D shows the number of plasma cells per spleen in HOD mice receiving 26 TACI CRD2-Fc, an Fc control, or PBS. FIG. 52E shows the number of plasma cells per spleen and bone marrow in HOD mice receiving 26 TACI CRD2-Fc, an Fc control, anti-CD20, or PBS. FIG. 52F shows hematocrit levels in HOD mice before receiving 26 TACI CRD2-Fc, an Fc control, or PBS Pre-4Aby, as described in FIG. 52B, on day 3 before administration of 26 TACI CRD2-Fc, an Fc control, or PBS (Day −3), and on days 15 and 28 post-administration of 26 TACI CRD2-Fc, an Fc control, or PBS. FIG. 52G shows autoantibodies (anti-globulin) bound to red blood cells (RBCs) in HOD mice receiving on day 4 before administration of 26 TACI CRD2-Fc, an Fc control, or PBS, and on days 9, 15, 23, and 28 post-administration of 26 TACI CRD2-Fc, an Fc control, or PBS.

FIGS. 53A-53F depict that 26 TACI CRD2-Fc provides benefit in an experimental autoimmune myasthenia gravis (EAMG) mouse model. FIG. 53A depicts a timeline of the EAMG mouse model. FIG. 53B depicts mean EAMG clinical scores across time. FIG. 53C depicts serum anti-AChR IgG concentration at Day 91 (end of study). FIG. 53D and FIG. 53E depict serum Ig isotype concentration at Day 91 (end of study). FIG. 53F depicts muscle AChR content at Day 91 (end of study).

FIGS. 54A-54E depict diagnostic plots for population pharmacokinetic analysis. FIG. 54A depicts PK model structure wherein Vc refers to the blood system and Vp refers to the tissue. A. FIG. 54B depicts individual predictions (μg/mL) versus observed concentration (μg/mL). FIG. 54C depicts population prediction (μg/mL) versus observed concentration (μg/mL). FIG. 54D depicts time (days) versus conditional weighted residual. FIG. 54E depicts population prediction (μg/mL) versus conditional weighted residual.

FIG. 55 depicts concentration of 26 TACI CRD2-Fc across 8 weeks following intravenous (IV) administration at doses 2.4 mg, 8 mg, 24 mg, 80 mg, 240 mg, 480 mg or 960 mg or subcutaneous (SC) administration at 80 mg, 240 mg, 480 mg or 960 mg.

FIG. 56 depicts a simulation showing concentration of 26 TACI CRD2-Fc across 40 weeks following subcutaneous (SC) administration at doses 24 mg, 80 mg and 240 mg. Dosing was repeated every 4 weeks (Q4W), every 8 weeks (Q8W) or every 12 weeks (Q12W) for a duration of 24 weeks of dosing and 16 weeks of post-dosing.

FIGS. 57A-57F depict PK and PD modeling for circulating APRIL and immunoglobulins (IgA, IgG, IgM). FIG. 57A depicts the PK/PD model structure for circulating free APRIL. FIG. 57B depicts PK/PD model structures for circulating IgA, IgM and IgG. FIG. 57C depicts free APRIL (μg/mL) across 12 weeks in response to IV (2.4 mg, 8 mg, 24 mg, 80 mg, 240 mg, 480 mg and 960 mg) or SC (80 mg, 240 mg, 480 mg and 960 mg) administration of 26 TACI CRD2-Fc. FIG. 57D depicts IgA (g/L) across 17 weeks in response to IV (2.4 mg, 8 mg, 24 mg, 80 mg, 240 mg, 480 mg and 960 mg) or SC (80 mg, 240 mg, 480 mg and 960 mg) administration of 26 TACI CRD2-Fc. FIG. 57E depicts IgG (g/L) across 17 weeks in response to IV (2.4 mg, 8 mg, 24 mg, 80 mg, 240 mg, 480 mg and 960 mg) or SC (80 mg, 240 mg, 480 mg and 960 mg) administration of 26 TACI CRD2-Fc. FIG. 57F depicts IgM (g/L) across 17 weeks in response to IV (2.4 mg, 8 mg, 24 mg, 80 mg, 240 mg, 480 mg and 960 mg) or SC (80 mg, 240 mg, 480 mg and 960 mg) administration of 26 TACI CRD2-Fc.

FIGS. 58A-58G depict PK/PD simulations for 26 TACI CRD2-Fc. FIG. 58A depicts free APRIL and APRIL as a percentage change from baseline across 40 weeks following subcutaneous (SC) dosing (80 mg or 240 mg) repeated every 4 weeks (Q4W). FIG. 58B depicts APRIL as a percentage change from baseline across 40 weeks following 24 weeks of subcutaneous (SC) dosing (24 mg, 80 mg, 240 mg) repeated every 4 weeks (Q4W), every 8 weeks (Q8W) or every 12 weeks (Q12W). FIG. 58C depicts IgA as a percentage change from baseline across 40 weeks following 24 weeks of subcutaneous (SC) dosing (24 mg, 80 mg, 240 mg) repeated every 4 weeks (Q4W), every 8 weeks (Q8W) or every 12 weeks (Q12W). FIG. 58D depicts IgG as a percentage change from baseline across 40 weeks following 24 weeks of subcutaneous (SC) dosing (24 mg, 80 mg, 240 mg) repeated every 4 weeks (Q4W), every 8 weeks (Q8W) or every 12 weeks (Q12W). FIG. 58E depicts IgM as a percentage change from baseline across 40 weeks following 24 weeks of subcutaneous (SC) dosing (24 mg, 80 mg, 240 mg) repeated every 4 weeks (Q4W), every 8 weeks (Q8W) or every 12 weeks (Q12W). FIG. 58F depicts free APRIL as a percentage change from baseline across 40 weeks following 24 weeks of subcutaneous (SC) dosing (24 mg, 80 mg, 240 mg) repeated every 4 weeks (Q4W), every 8 weeks (Q8W) or every 12 weeks (Q12W); and depicts IgA, IgG, and IgM as a percentage change from baseline across 72 weeks following 24 weeks of subcutaneous (SC) dosing (24 mg, 80 mg, 240 mg) repeated every 4 weeks (Q4W), every 8 weeks (Q8W) or every 12 weeks (Q12W). FIG. 58G depicts free IgG and IgG as a percentage change from baseline across 72 weeks following subcutaneous (SC) dosing (80 mg or 240 mg) repeated every 4 weeks (Q4W).

FIGS. 59A-59D depict PK and PD modeling for circulating BAFF and Gd-IgA1. FIG. 59A depicts the PK/PD model structure for circulating free BAFF. FIG. 59B depicts PK/PD model structures for circulating Gd-IgA1. FIG. 59C depicts free BAFF (μg/mL) across 16 weeks in response to IV (2.4 mg, 8 mg, 24 mg, 80 mg, 240 mg, 480 mg and 960 mg) or SC (80 mg, 240 mg, 480 mg and 960 mg) administration of 26 TACI CRD2-Fc. BLQ=Below Limit of Quantitation. FIG. 59D depicts Gd-IgA1 (μg/mL) across 16 weeks in response to IV (2.4 mg, 8 mg, 24 mg, 80 mg, 240 mg, 480 mg and 960 mg) or SC (80 mg, 240 mg, 480 mg and 960 mg) administration of 26 TACI CRD2-Fc.

FIGS. 60A-60B depict PK/PD simulations for 26 TACI CRD2-Fc. FIG. 60A depicts free APRIL and APRIL as a percentage change from baseline across 40 weeks following subcutaneous (SC) dosing (80 mg or 240 mg) repeated every 4 weeks (Q4W). FIG. 60B depicts free Gd-IgA1 and Gd-IgA1 as a percentage change from baseline across 72 weeks following subcutaneous (SC) dosing (80 mg or 240 mg) repeated every 4 weeks (Q4W).

FIGS. 61A-61B depict a summary of target coverage in simulations. FIG. 6IA shows patients with >95% APRIL coverage. FIG. 61B shows the percentage of patients with IgG below 1.5 g/L.

FIGS. 62A-62B depict a summary of target coverage in simulations for APRIL/BAFF and IgG/Gd-IgAQ1. FIG. 62A shows patients with 95% APRIL/BAFF coverage. FIG. 62B predicts that patients will have decreases in Gd-IgAQ1 greater than 50%.

FIGS. 63A-63E show proteinuria and urinary creatinine scores over time and at Day 48 in the IFNα-accelerated NZB/W lupus model. FIG. 63A shows mean proteinuria scores (+SD) for each treatment group over time, with the last observation carried forward (LOCF) for any mouse terminated before day 50. FIG. 63B shows mean creatinine scores (+SD) over time, with LOCF for any mouse terminated before day 50. FIG. 63C shows proteinuria scores at Day 48 (the last measurement prior to termination) by treatment group. FIG. 63D shows creatinine scores at day 48 by treatment groups. FIG. 63E shows the ratio of proteinuria to urinary creatinine score at Day 48. Data are presented as median±IQR for FIGS. 63C-63E. Study details and methodology provided in Example 32. Statistical differences between each group were determined by Kruskal-Wallis test with uncorrected Dunn's test for multiple comparisons as described in Example 32; only significant differences (p<0.05 are listed.

FIGS. 64A-64F show histological analysis of kidney, spleen, sub-mandibular gland, and lacrimal gland at termination (Day 50) in the IFNα accelerated NZB/W lupus model. FIG. 64A shows total glomerular lesion scores in kidney. FIG. 64B shows total tubular and interstitial lesion scores in kidney. FIG. 64C shows average follicular diameter in spleen. FIG. 64D shows spleen follicle score. FIG. 64E shows number of inflammatory foci in the submandibular gland. FIG. 64F shows number of inflammatory foci in the lacrimal gland. Data are presented as median±IQR. Study details and methodology provided in Example 32. Statistical differences between each group were determined by Kruskal-Wallis test with uncorrected Dunn's test for multiple comparisons as described in Example 32; only significant differences p<0.05 are listed.

FIGS. 65A-65E show hemoglobin (HGB), Hematocrit (HCT), RBC, and direct antiglobulin test (DAT) levels in whole blood at termination (day 50) in the IFNα accelerated NZB/W lupus model. FIG. 65A shows hemoglobin concentrations. FIG. 65B shows hematocrit (%). FIG. 65C shows RBC concentrations. FIG. 65D shows pan Ig DAT levels (MFI). FIG. 65E shows IgA DAT levels (MFI). MFI: mean fluorescence intensity recorded using flow cytometry. Data are presented as median±IQR. Study details and methodology provided in Example 32. Statistical differences between each group were determined by Kruskal-Wallis test with uncorrected Dunn's test for multiple comparisons as described in Example 32; only significant differences p<0.05 are listed.

FIGS. 66A-66B show Blood urea nitrogen (BUN) and anti-double stranded (ds) DNA IgM levels in serum at termination (day 50) in the IFNα accelerated NZB/W lupus model. FIG. 66A shows BUN concentration. FIG. 66B shows anti-dsDNA IgM level. Data are presented as median±IQR. Study details and methodology provided in Example 32. Statistical differences between each group were determined by Kruskal-Wallis test with uncorrected Dunn's test for multiple comparisons as described in Example 32; only significant differences p<0.05 are listed.

FIGS. 67A-67C show H&E scoring, serum Ig, and B cell frequencies in an experimental model of epidermolysis bullosa acquisita (EBA). FIG. 67A shows mice in the EBA model treated with 26 TACI CRD2-Fc starting when disease covered at least 2% of surface area, had significantly lower H&E scores of dermis vs. Fc control-treated mice at termination. P value shown from the Mann-Whitney test. FIG. 67B shows serum immunoglobulins, both total and collagen VII-specific isotypes. P value shown from the Mann-Whitney test. FIG. 67C shows the frequences of COL7^{vWFA2}-(antigen)-specific B cells in the lymph nodes of 26 TACI CRD2-Fc-treated mice. Statistically significant differences between Fc control and 26 TACI CRD2-Fc treatment groups were determined by the Mann-Whitney test. ****p<0.0001; ***p<0.001; **p<0.01; *p<0.05.

FIGS. 68A-68B show serum levels of anti-GluN1 peptide IgG antibodies at Week 4 (prior to treatment initiation) and at termination (Week 10). P values shown for statistical differences between treatment groups determined using the Mann-Whitney test at each serum dilution.

### DETAILED DESCRIPTION

Provided herein are immunomodulatory proteins that engage with one or more ligand, e.g. produced as soluble factors, to suppress or reduce B cell responses or activity. Among the provided immunomodulatory proteins are proteins that bind to BAFF or APRIL ligands to neutralize their activity and block or antagonize the activity of B cell stimulatory receptors, such as TACI or BCMA. The provided immunomodulatory proteins may be fusion proteins of a TACI extracellular domain or binding portion thereof (hereinafter TACI ECD) and a multimerization domain, such as an immunoglobulin Fc. For example, provided herein are TACI-Fc fusion proteins. In some embodiments, the immunomodulatory proteins provided herein can be used for the treatment of diseases, disorders or conditions that are associated with a dysregulated immune response, such as associated with inflammatory or autoimmune symptoms including an inflammatory disease or an autoimmune disease.

The immune system relies on immune checkpoints to prevent autoimmunity (i.e., self-tolerance) and to protect tissues from excessive damage during an immune response, for example during an attack against a pathogenic infection. In some cases, however, the immune system can become dysregulated and an abnormal immune response can be mounted against a normal body part or tissue, resulting in an autoimmune disease or condition or autoimmune symptoms. In other cases, an unwanted immune response can be mounted to a foreign tissue, such as a transplant, resulting in transplant rejection.

B cells have long been implicated in autoimmune diseases such as systemic lupus erythematosus (SLE), owing to their ability to present antigen to autoreactive T cells, secrete inflammatory cytokines (Lund, Curr Opin Immunol 2008, 20(3):332-338), and differentiate into antibody-secreting cells (ASC), i.e., plasmablasts and plasma cells (PC) that are responsible for the production of pathogenic autoantibodies (Banchereau et al., Cell, 2016, 165(3):551-565). Therefore, depletion or inhibition of B cells and ASC represent a compelling approach for many rheumatic and other autoimmune disorders.

Similarly, since B cells are crucial mediators of systemic immune responses, B cells also play a role in renal, hematologic, dermatologic, and neurologic autoimmune diseases.

In the immunopathogeneisis of IgA nephropathy (IgAN), a form of renal disease, B cells produce small amounts of antibodies (e.g., Gd-IgAQ1) and plasma cells produce high amounts of autoantibodies (e.g., anti-Gd-IgA1). This leads to formation of antibody: autoantibody comlpexes that deposit and accumulate in mesangial cells, which activates the alternative and lectin pathways of the complement system leading to chronic inflammation, loss of renal function, hematuriea, proteinuria and reduction in glomerular filtration rate (Maixnerova et a., (2022) J Clin Med, 11(10):2810). Thus, targeting BAFF and APRIL have emerged as a promising approach for reducing levels of pathogenic autoantibodies (e.g., Gd-IgAQ1).

In autoimmune cytopenias, targeting BAFF and APRIL can lead to the reduction of pathogenic autoantibodies that cause destruction of platelets in immune thrombocytopenia (ITP), and destruction of red blood cells in warm autoimmune hemolytic anemia (wAIHA) and cold autoimmune hemolytic anemia (cAIHA or CAD).

B cells are known to be substantial in the pathogenesis of autoimmune diseases with cutaneous manifestations. Among these autoimmune diseases are autoimmune blistering diseases, lupus erythematosus, dermatomyositis, systemic sclerosis, psoriasis, pemphigus, and pemphigoid, the latter two being particularly driven by authoantibodies (Fetter et al., Cells (2020) 9(12):2627). Traditionally, skin was believed to be devoid of B cells. However, recent data has shown that B cells localize to the skin of humans and other mammalian species (Debes and McGettigan, J Immunol (2019) 202(6):1659-1666. Once localized to skin, autoreactive skin-associated B cells can contribute locally to autoantibody production, cytokine expression, and crosstalk to autoreactive T cells (Fetter et al., Cells (2020) 9(12):2627).

Autoimmune blistering diseases (ABDs) are characterized by autoantibodies targeting structural skin proteins. Treatments are limited: rituximab is the only biologic approved for pemphigus vulgaris (Uzawa et al. (2021) Clin Exp Immunol 203:366; Ma et al. (2023) Front Immunol 13:1064007), but may be associated with frequent relapses, often accompanied by elevations in the cytokine BAFF3. BAFF and its related cytokine APRIL play key roles in B-cell activation across a broader spectrum of B cells than rituximab and are elevated in ABDs, correlating with disease activity. BAFF/APRIL inhibition may lead to more durable autoantibody reductions, improving clinical outcomes.

For autoantibody-mediated neurologic diseases, there is a rapidly expanding and clinically distinct group of central nervous system (CNS) diseases that are caused by pathogenic autoantibodies. Some of these autoantibodies target neuroglial surface proteins. Autoantigen-specific B cells have been consistently identified in the circulation of patients with neuroglial surface autoantibody (NSAb)-mediated diseases (Sun et al, Nat Rev Neurol, (2020) 16(9):481-492). The efficacy of certain anti-B cell therapies has been detailed for the treatment of patients with multiple sclerosis, neuromyelitis-spectrum disorders, autoimmune encephalitis and hyperexcitability CNS disorders, autoimmune neuropathies, myasthenia gravis, and inflammatory myopathies (Stathopoulos and Dalakas, Neurotherapeutics, (2022) 19(3):691-710). Even more specifically, mysathenia gravis (MG) is an archetypal B cell-mediated autoimmune disorder in that the presence of autoantibodies that specifically target components of the acetylcholine receptor (AChR) impairs neuromuscular transmission in the postsynaptic membrane (Yi et al., Muscle Nerve (2018) 57(2):172-184.

Further, BAFF and APRIL play key roles in B cell biology. One or both cytokines have been reported to be upregulated and associated with clinical parameters of MG, autoimmune encephalitis, NMOSD, MS, and other autoantibody-related neurological diseases (Uzawa et al. (2021) Clin Exp Immunol 203:366; Ma et al. (2023) Front Immunol 13:1064007; Ashida et al. (2022) Front Neurol 13:1012857). Therapeutic agents targeting B cell pathways, including BAFF and APRIL, have demonstrated promising clinical potential in the treatment of myasthenia gravis (MG), as well as other autoantibody-related neurological diseases; however, there is still need for more safe and efficacious therapies. Targeting BAFF and APRIL can reduce the levels of pathogenic autoantibodies, (e.g., anti-NMDAR, anti-AChR, anti-MOG) and autoantibodies to proteins at the neuromuscular junction or other sites of neuron-neuron or neuron-tissue interaction.

Therefore, depletion or inhibition of B cells and ASC represent a compelling approach for many renal, hematologic, dermatologic, and neurologic autoimmune disorders.

Key modulators of B cell development, differentiation, and survival include the tumor necrosis factor (TNF) family cytokines, B cell activating factor (BAFF/TNFSF13B) and a proliferation-inducing ligand (APRIL/TNFSF13), which are expressed primarily by myeloid cells and signal through multiple receptors. BAFF binds with varying affinity to B cell-expressed BAFF-R (TNFRSF13C), transmembrane activator and calcium-modulating cyclophilin ligand interactor (TACI; TNFRSF13B), and B cell maturation antigen (BCMA; TNFRSF17), while APRIL binds TACI and BCMA (Samy, et al., Int Rev Immunol, 2017, 36(1):3-19), heparin sulfate proteoglycans (HSPG) (Ingold, et al., J Exp Med, 2005, 201(9):1375-1383), for example, Syndecans like CD138 (Moreaux et al., Eur J Haematol, 2009, 83(2):119-129; Ingold, et al., J Exp Med, 2005, 201(9):1375-1383). BAFF can exist in three functional forms: membrane-bound, soluble trimer, and soluble BAFF 60-mer (Eslami and Schneider, Curr Opin Immunol, 2021, 71:75-80), with the soluble trimer formed via proteolytic cleavage of membrane BAFF (Samy, et al., Int Rev Immunol, 2017, 36(1):3-19). APRIL and BAFF can also form functionally active heterotrimers; all forms of these cytokines have been shown to be elevated in various antibody-related diseases, including SLE (Roschke et al., J Immunol, 2002, 169(8):4314-4321; Dillon et al., Arthritis Res Ther, 2010, 12(2):R48).

Accordingly, BAFF and APRIL are TNF superfamily members that bind both TACI and BCMA receptors on B cells; BAFF also binds a 3^{rd} receptor, BAFF receptor (BAFF-R). Both BAFF and APRIL can bind and activate BCMA and TACI; BAFF also binds and activates the BAFF-R (Xu et al. 2020 Cancers (Basel) 12(4):1045). Together, BAFF and APRIL support B cell development, differentiation, and survival, particularly for plasmablasts and plasma cells, and play a role in the pathogenesis of B cell-related autoimmune diseases. BAFF and APRIL are initially expressed as transmembrane proteins, primarily on stromal cells and cells of myeloid origin (Smulski et al. Front. Immunol. 2018 9:2285) and can be cleaved to release soluble cytokines. BAFF circulates as homotrimers, as 60-mers, or as a heterotrimers containing 2 APRIL and 1 BAFF, or 2 BAFF and 1 APRIL protomers. APRIL circulates as homo- or heterotrimers and can be localized to the intracellular matrix or cell surfaces through interaction with heparin sulphate proteoglycans.

Despite structural similarities and engagement of common signaling pathways, APRIL and BAFF play non-redundant roles in B cell regulation, due in part to differential receptor expression at partially overlapping stages of B cell development. While BAFF plays key roles earlier in B cell development when BAFF-R is expressed, APRIL assumes a key role in the function of differentiated ASC that express TACI, BCMA, and HSPG (e.g., syndecan-1/CD138).

The expression of BAFF and APRIL increases under proinflammatory conditions (Smulski et al. 2018), and elevated serum levels of these cytokines have been correlated with disease severity in patients with B cell-related autoimmune disease, including systemic lupus erythematosus (SLE) (Samy et al. Int. Rev. Immunol. 2017 36:3-19). Binding of BAFF/APRIL to their receptors triggers events in B cell and plasma cell development, differentiation, and activation. For instance, activation of the BAFF-R contributes to survival and maturation of transitional and naïve B cells whereas TACI is involved in T cell-independent B cell responses to certain antigens, B cell regulation, and immunoglobulin (Ig) class-switch recombination. BCMA, which is upregulated in activated B cells, is important for the long-term survival of plasma cells.

In some aspects immunotherapy that alters immune cell activity, such as B cell activity, can treat certain diseases, disorders and conditions in which the immune response is dysregulated. In particular, inhibition or attenuation of an immune response, such as a B cell response, could be desirable to reduce or prevent unwanted inflammation, autoimmune symptoms and/or transplant rejection. Therapeutic approaches that seek to modulate interactions between ligands and their receptors that mediate an immune response, however, are not entirely satisfactory. In some cases, therapies to intervene and alter the immunomodulatory effects of immune cell, e.g. B cell, activation are constrained by the spatial orientation requirements as well as size limitations imposed by the confines of the immunological synapse. In some aspects existing therapeutic drugs, including antibody drugs, may not be able to interact simultaneously with the multiple target proteins involved in modulating these interactions. For example, soluble receptors and antibodies generally bind competitively (e.g., to no more than one target species at a time) and therefore lack the ability to simultaneously bind multiple targets. Additionally, pharmacokinetic differences between drugs that independently target one of these receptors can create difficulties in properly maintaining a desired blood concentration of a drug combination targeting two different targets throughout the course of treatment.

Inhibitors of BAFF and/or APRIL have been investigated in clinical trials for the treatment of a variety of autoimmune or other B-cell related diseases. An inhibitor of BAFF, belimumab (Benlysta®) has been approved for treatment of SLE (Benlysta Product Information, 2020), and single-pathway inhibitors of APRIL (e.g., BION1301 and VIS649) are currently being evaluated in Phase 2 studies [NCT04684745; NCT04287985].

Among several B cell targeting strategies, blockade of BAFF or APRIL has shown clinical promise. Belimumab is an anti-BAFF antibody approved for the treatment of SLE (Hahn, N Engl J Med, 2013, 368(16):1528-1535) and SLE-related lupus nephritis (LN) (Asif et al., Curr Opin Nephrol Hypertens, 2022), but clinical remission as measured by Lupus Low Disease Activity State (LLDAS) or Complete Renal Response (CRR) is achieved in only a minority of patients, (12-4% or 30%, respectively) (Oon et al., Ann Rheum Dis, 2019, 78(5):629-633; Furie et al., N Engl Med, 2020, 383(12):1117-1128). Thus, there remains a need for more active agents. Other BAFF/APRIL-targeting antibodies include ianalumab, a blocking and cell-depleting anti-BAFF-R antibody (McWilliams et al., Blood Adv, 2019, 3(3):447-460), and the anti-APRIL antibodies BION-1301 (Dulos, America Society of Hematology, 2016) and sibeprenlimab (VIS649) (Myette et al., Kidney Int, 2019, 96(1):104-116). These antibodies have demonstrated promising pharmacodynamic activity in Phase 1 clinical trials (Barratt, American Society of Nephrology, 2021; Mathur et al., Kidney Int Rep, 2022, 7(5):993-1003), but are limited by only inhibiting either BAFF or APRIL (Ramanujam et al., J Clin Invest, 2006, 116(3):724-734; Benson et al., J Immunol, 2008, 180(6):3655-3659; Liu et al., Exp Cell Res, 2011, 317(9):1270-1277; Huard et al., PloS One, 2012, 7(2):e31837, Haselmayer et al., Eur J Immunol, 2017, 47(6):1075-1085; Samy et al., Int Rev Immunol, 2017, 36(1):3-19; Stohl et al., Arthritis Rheumatol, 2020, 72(2):292-302). BAFF-Trap, a WT TACI and WT BAFF-R hybrid Fc-fusion protein (Zhou et al., Signal Transduct Target Ther, 2019, 4:19) also shares limitations by inhibiting only BAFF.

The co-neutralization of BAFF and APRIL dramatically reduces B cell function, including antibody production, whereas inhibition of either BAFF or APRIL alone mediates relatively modest effects. Fc fusions of wild-type (WT) extracellular domain of TACI and the Fc domain of IgG1 (e.g. atacicept and telitacicept) are in clinical development and target both BAFF and APRIL. Atacicept (Samy et al., Int Rev Immunol, 2017, 36(1):3-19) and telitacicept (Shi et al., Immunopharmacol Immunotoxicol, 2021, 1-8) are soluble WT TACI extracellular domain (ECD) Fc-fusion proteins that strongly inhibit BAFF and weakly inhibit APRIL signaling.

These dual BAFF/APRIL antagonists have been shown to inhibit the survival of immature and mature B cells and plasma cells, while sparing B cell progenitors and memory B cells (Cogollo et al. 2015 Drug Des Devel Ther. 9:1331-9; Samy et al. 2017; Zhao et al. 2016 J Clin Pharmacol. 56:948-959). Levels of serum IgG, IgM, and IgA and numbers of mature and total circulating B cells are reduced by both (Coggollo e al. 2015; Chen et al. 2014 Clin Pharmacokinet. 53:1033-44; Chen et al. 2016 Br J Clin Pharmacol. 82:41-52; Zhao et al. 2016). When compared directly to inhibition of either BAFF or APRIL alone in nonclinical studies, dual inhibitors have shown more pronounced pharmacodynamic (PD) effects and greater modification of disease models (Ramanujam et al. 2006 J Clin Invest. 116:724-34; Benson et al. 2008 J Immunol. 180:3655-3659; Haselmeyer et al. 2017 Eur J. Immunol. 47:1075-1085; Samy et al. 2017). Atacicept and telitacicept have demonstrated promising clinical potential in certain autoimmune diseases e.g. systemic lupus erythematosus (SLE) and IgA nephropathy but have not yet clearly exhibited long-term and/or complete disease remissions. For instance, Atacicept and telitacicept have both demonstrated clinical activity in SLE (Merrill et al., Arthritis Rheumatol, 2018, 70(2):266-276; Dhillon, Drugs, 2021; Shi et al., Immunopharmacol Immunotoxicol, 2021, 1-8). However, atacicept formally failed to meet its primary endpoint in pivotal trials (Merrill et al., Arthritis Rheumatol, 2018, 70(2):266-276) and appears to no longer be in active development for SLE (Vera, Therapeutics Provides Business Update and Reports Second Quarter 2022 Financial Results, 2022). In contrast, telitacicept has been conditionally approved in China for the treatment of SLE based on a phase 2b study, and recently reported positive confirmatory phase 3 results; however, most subjects appear to have still flared within the first 6 months of treatment (Wu et al., American College of Rheumatology, 2019).

While B cell targeting therapies have demonstrated promising therapeutic potential, they are not entirely satisfactory. Until now, co-targeting BAFF and APRIL has been attempted only with development of the WT TACI-Fc molecules atacicept and telitacicept, though the affinity of WT TACI-Fc for APRIL is arguably suboptimal, well below that achieved by anti-APRIL mAbs, which range in affinity from KD=0.95 to 400 pM, depending on the method used (Dulos, America Society of Hematology, 2016; Myette et al., Kidney Int, 2019, 96(1):104-116). For instance, soluble recombinant TACI (e.g. atacicept or telitacicept) demonstrates considerable promise as a therapeutic, but its usefulness appears hindered by low to moderate affinity to APRIL. Thus, while these molecules arguably neutralize BAFF sufficiently, their inefficient blockade of APRIL activity leaves clear room for improvement. These findings provide clinical validation of the BAFF/APRIL pathway for SLE, but also suggest that further improvement upon the drug designs of atacicept and telitacicept, perhaps by improving APRIL inhibition in particular, may afford a unique opportunity to achieve more effective yet safe therapeutic options.

Among provided embodiments are those that provide for improved neutralizing activity and suppression or reduction of B cell responses. In some embodiments, the improved activity is mediated by increased or improved binding or interaction of the provided immunomodulatory proteins (e.g. TACI-Fc fusion protein) with BAFF and/or APRIL. The provided immunomodulatory proteins block or antagonize interactions of BAFF or APRIL, such as homotrimers of BAFF or APRIL, heterotrimers of BAFF/APRIL or BAFF 60mers, with a cognate B cell stimulatory receptor, and thereby neutralize activity of BAFF and/or APRIL ligands. In some embodiments, the provided immunomodulatory proteins reduce one or more B cell response or activity, including the ability of B cells to produce immunoglobulins. In some embodiments, the provided immunomodulatory proteins (e.g. TACI-Fc fusion protein), when administered to a subject, reduce circulating serum immunoglobulins. In some embodiments, the provided immunomodulatory proteins reduce one or more of B cell maturation, differentiation and proliferation. In provided aspects, such activity is improved or superior to that achieved by a WT TACI-Fc fusion protein (e.g. telitacicept or atacicept). In some embodiments, the provided immunomodulatory proteins (TACI-Fc fusion protein) are candidate therapeutics for the treatment of multiple autoimmune and inflammatory diseases, particularly B cell-related diseases, such as SLE, SjS, and other connective tissue diseases.

Provided embodiments include methods and uses of a particular Fc fusion protein of a TACI variant TNF receptor domain (TD, i.e. CRD2) that simultaneously inhibits the BAFF and APRIL cytokines. Provided embodiments relate to identification of variant TACI polypeptides engineered to have improved affinity towards APRIL and/or BAFF following random mutagenesis and directed evolution of the second cysteine rich domain (CRD2) of TACI, spanning residues 68-110. As shown herein, the affinity maturation included five selections alternating between APRIL and BAFF, with concurrent decreases in selection reagent concentration to maintain selection pressure. Results demonstrated variant TACI polypeptides that exhibit substantially enhanced affinity for BAFF and APRIL as compared to wild-type TACI. For example, provided herein are variant TACI polypeptides that contain one or more amino acid substitutions (replacement or mutations) that confer improved binding affinity of the protein for BAFF and/or APRIL. In particular, among provided embodiments are those that provide for improved, combined BAFF and APRIL inhibition. Thus, the provided immunomodulatory proteins provide effective and durable disease suppression in the treatment of autoimmune or inflammatory diseases, including in severe B cell-related autoimmune diseases like SLE.

For example, the provided embodiments are based on findings that directed evolution by affinity modification of TNFR domain (TD) of the ectodomain of TACI facilitated the development of molecules with improved affinity for APRIL and/or BAFF. Thus, the affinity modification produces a variant TACI that contains a variant TNFR domain (vTD). Fusion of such molecules with an immunoglobulin Fc results in immunomodulatory proteins that suppress B cell activity and response. For instance, reformatted as a soluble Fc fusion protein, the affinity-matured TACI variant outputs exhibited inhibition of APRIL and BAFF, as shown herein in a TACI-dependent reporter assay, and with lower IC₅₀ values than wild-type TACI-Fc and belimumab comparators. Further, results in evaluated animal models demonstrate rapid and significantly reduced key lymphocyte subsets including plasma cells, germinal center B cells, and follicular T helper cells. Further, tested variant molecules exhibited improved activities in mouse models, including significantly reduced autoantibodies and sialadenitis in the spontaneous SjS model, inhibited glomerular IgG deposition in the bm12-induced model of lupus, and potently suppressed anti-dsDNA autoAbs, blood urea nitrogen levels, proteinuria, sialadenitis, kidney lesions and renal immune complex deposition in the NZB/W lupus model. Further, as compared to wild-type TACI-Fc, tested TACI-Fc fusions exhibited significantly and persistently decreased titers of serum IgM, IgG, and IgA antibodies in mice. The findings herein demonstrate these immunomodulatory proteins consistently exhibit potent immunosuppressive activity and efficacy in vitro and in vivo, appearing superior to existing and/or approved immunomodulators like belimumab, abatacept, atacicept, or telitacicept. Such biologics may therefore be attractive development candidates for the treatment of serious autoimmune and/or inflammatory diseases, including B cell-related diseases such as SLE, Sjogren's syndrome, and other connective tissue diseases.

Moreover, observations herein demonstrate that the TACI-Fc fusion proteins exhibit high serum exposure when administered to mice and cynomolgus monkeys. The favorable and higher serum exposure, as well as the more potent immunosuppressive activities, achieved by the provided TACI-Fc fusion proteins supports their use at a lower clinical dose and/or at a reduced dosing frequency (or longer dosing interval) than existing WT TACI-Fc therapeutics. For instance, existing WT TACI-Fc therapeutics, such as telitacicept an atacicept, must be administered at least once weekly. Reducing the dose frequency may provide a treated subject with better symptom control, improve adherence to the dosing regimen, increase patient quality of life or patient satisfaction and/or overall reduce the costs of receiving the treatment. Moreover, reducing the dose, even at a more regular frequency such as once weekly, may also mitigate against certain adverse effects.

In particular embodiments, the provided TACI-Fc fusion proteins are for treating SLE and other autoantibody-related rheumatic diseases for which there remain indications of high unmet need. In SLE, treatment options have been hindered by complex pathogenesis and heterogeneity of disease, suggesting that multiple pathways or aspects of B cell development and differentiation may require simultaneous inhibition to enable durable responses. While B cell-depleting agents such as rituximab/ocrelizumab/obinutuzumab (anti-CD20), and obexelimab (anti-CD19) have exhibited favorable clinical impacts in certain autoimmune disease settings, this has not translated to SLE, where rituximab failed to demonstrate benefit in SLE and LN trials (Merrill et al., Arthiritis Rheum, 2010, 62(1):222-233; Rovin et al., Arthritis Rheum, 2012, 64(4):1215-1226). One possible limitation of these therapeutics is that CD20 and CD19 are not expressed on all ASC or LL-PC, and only earlier stage B cells (including pro/pre, immature, mature, and memory B cells) are depleted, sparing most pathogenic plasmablasts and PC (Lee et al., Nat Rev Drug Discov, 2021, 20(3):179-199; Arbitman et al., J Autoimmun, 2022, 102873).

Targeting or co-targeting BAFF and/or APRIL is an alternative to ADCC-mediated B cell depletion. Preclinical studies have demonstrated that starving B cells of these two critical B cell survival and differentiation factors can significantly reduce all B cell subsets beyond the immature T1 stage of development, including LL-PC, without affecting CD19+CD20+ pro/pre-B cell precursors (Gross et al., Immunity, 2001, 15(2):289-302). Inhibition of ASC can dramatically impact pathogenic antibody production and thereby potentially reduce disease activity. Although early efforts to target the BAFF/APRIL pathway focused on agents like belimumab that neutralize only BAFF, inhibition of both APRIL and BAFF may be required to impact survival of more differentiated, pathogenic TACI+/BCMA+ ASC (Samy et al., Int Rev Immunol, 2017, 36(1):3-19).

APRIL plays a particularly important role in IgA class switching, production, and glycosylation, as first indicated by studies of APRIL knockout mice (Castigli et al., Proc Natl Acad Sci USA, 2004, 101(11):3903-3908). In addition, elevated plasma APRIL levels in IgA nephropathy (IgAN) patients are associated with more severe clinical manifestations such as high proteinuria and Gd (galactose deficient) IgAQ1 levels (Zhai et al., Medicine (Baltimore), 2016, 95(11):e3099), which are important causal factors and contribute to disease pathogenesis. Indeed, early trials of BION-1301 and sibeprenlimab suggest that APRIL-only inhibition can mediate significant decreases in Ig (particularly IgA) in healthy subjects, and BION-1301 impacts proteinuria in IgAN patients in an ongoing trial (Barratt et al., J Immunol, 2022, 180(6):3655-3659). However, targeting APRIL alone has its own limitations and would not be expected to impact less mature BAFF-dependent B cells that can also contribute to disease pathogenesis (Lee et al., Nat Rev Drug Discov, 2021, 20(3):179-199). BAFF neutralization leads to downregulation of B cell function, decreases in autoantibody production, and inhibition of tertiary lymphoid structure formation in the kidney (Samy, et al., Int Rev Immunol, 2017, 36(1):3-19).

Belimumab was the first approved therapy for SLE and LN (Lee et al., Nat Rev Drug Discov, 2021, 20(3):179-199), underscoring the need for new therapies. Another development in SLE therapy was the recent approval of anifrolumab an anti-type I interferon receptor antibody (Morand et al., N Engl J Med, 2020, 382(3):211-221; Deeks, Drugs, 2021). Anifrolumab targets a distinct pathophysiology of SLE from B cell modulators, by targeting myeloid dendritic cells rather than B cells, although type I interferons are known to indirectly promote B cell differentiation and loss of tolerance. IFN-regulated gene expression is significantly increased in SLE; however, interferon gene signature expression has not been predictive of response, underscoring the pleiotropic effects of the IFN system (Morand et al., N Engl J Med, 2020, 382(3):211-221). In contrast, the presence of high serum levels of BAFF and APRIL in patients with SLE is well established and has been described in numerous studies (Samy, et al., Int Rev Immunol, 2017, 36(1):3-19). High serum BAFF levels also correlate with elevated autoantibody levels, particularly anti-dsDNA Abs (Samy, et al., Int Rev Immunol, 2017, 36(1):3-19).

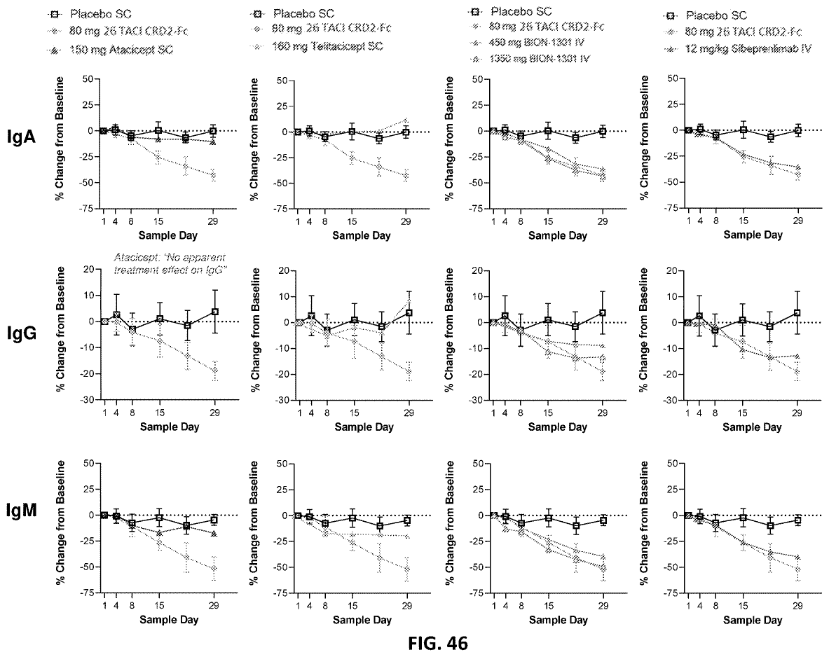

The provided TACI-Fc fusion protein is a potential best-in-class BAFF/APRIL inhibitor for SLE and other autoantibody-related diseases. The provided TACI-Fc fusion protein: has significantly improved ligand affinity; is superior to WT TACI-Ig, BAFF and/or APRIL-only inhibitors; and is well-tolerated in healthy adults via IV or SC administration with dose-dependent PK/PD. For example, FIG. 46 of the present disclosure illustrates the superiority of the provided TACI-Fc fusion protein in reducing circulating immunoglobulins (i.e., IgA, IgG and IgM) compared to current biologics. This demonstrates that the provided TACI-Fc fusion proteins are suitable for multiple autoantibody-related inflammatory diseases.

Additionally, TACI-Fc fusion proteins are well tolerated at low doses (e.g., 80 mg) to high doses (e.g., 960 mg) without adverse effects. The TACI-Fc fusion proteins are also well tolerated when administered once every four weeks (Q4W). Furthermore, the TACI-Fc fusion proteins are effective whether injected SC or IV at low doses (e.g., 80 mg).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in the specification and the appended claims, the singular forms “a,” “an,” and “the” include plural referents unless the context clearly indicates otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

The term “affinity-modified” as used in the context of a domain of a protein means a mammalian protein having an altered amino acid sequence in an extracellular domain or a specific binding portion thereof (relative to the corresponding wild-type parental or unmodified domain) such that it has an increased or decreased binding activity, such as binding affinity, to at least one of its binding partners (alternatively “counter-structures”) compared to the parental wild-type or unmodified (i.e., non-affinity modified domain) protein. In some embodiments, the affinity-modified domain can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences, such as amino acid substitutions, in a wild-type or unmodified domain. An increase or decrease in binding activity, e.g. binding affinity, can be determined using well known binding assays, including flow cytometry. Larsen et al., American Journal of Transplantation, Vol 5: 443-453 (2005). See also, Linsley et al., Immunity, 1: 7930801 (1994). An increase in a protein's binding activity, e.g. affinity, to its binding partner(s) is to a value at least 10% greater than that of the wild-type control and in some embodiments, at least 20%, 30%, 40%, 50%, 100%, 200%, 300%, 500%, 1000%, 5000%, or 10000% greater than that of the wild-type control value. A decrease in a protein's binding activity, e.g. affinity, to at least one of its binding partner is to a value no greater than 90% of the control but no less than 10% of the wild-type control value, and in some embodiments no greater than 80%, 70% 60%, 50%, 40%, 30%, or 20% but no less than 10% of the wild-type control value. An affinity-modified protein is altered in primary amino acid sequence of the extracellular domain or a specific binding portion thereof by substitution, addition, or deletion of amino acid residues. The term “affinity-modified” is not to be construed as imposing any condition for any particular starting composition or method by which the affinity-modified protein was created. Thus, an affinity-modified protein is not limited to wild-type protein domains that are then transformed to an affinity-modified domain by any particular process of affinity modification. An affinity-modified domain polypeptide can, for example, be generated starting from wild-type mammalian domain sequence information, then modeled in silico for binding to its binding partner, and finally recombinantly or chemically synthesized to yield the affinity-modified domain composition of matter. In but one alternative example, an affinity-modified domain can be created by site-directed mutagenesis of a wild-type domain. Thus, affinity modified TD domain denotes a product and not necessarily a product produced by any given process. A variety of techniques including recombinant methods, chemical synthesis, or combinations thereof, may be employed.

The term “affinity-modified TD domain” refers to an affinity-modified domain of a member of the tumor necrosis receptor superfamily (TNFRSF) protein or a TNF ligand thereof having an altered amino acid sequence of a TNFR domain or of a TNF domain therein, respectively. For example, an affinity-modified TD domain of a TNFRSF protein has an altered amino acid sequence of a TNFR domain composed of at least one cysteine rich domain (CRD) within the extracellular domain of the TNFRSF protein or a specific binding portion thereof (relative to the corresponding wild-type parental or unmodified domain) such that it has an increased or decreased binding activity, such as binding affinity, to at least one of its binding partners (alternatively “counter-structures”) compared to the parental wild-type or unmodified protein containing the non-affinity modified or unmodified TD domain.

An “affinity-modified TACI” (also referred to as a variant TACI) refers to a TACI protein molecule that antagonizes or blocks the activity of a B cell stimulatory receptor. For example, TACI binds to APRIL and/or BAFF, which are ligands of the B cell stimulatory receptors B cell maturation antigen (BCMA), B cell activation factor receptor (BAFF-R), and transmembrane activator and calcium modulator and cyclophilin ligand interactor (TACI). In particular embodiments, a BIM includes the extracellular domain of TACI, or a portion of the extracellular domain of TACI containing a TNF receptor family domain (e.g. TD, e.g. CRD) that binds to cognate ligands APRIL and/or BAFF, and heterotrimers of APRIL and BAFF. An affinity-modified variant of the extracellular domain or portion thereof of TACI can include one more amino acid modifications (e.g. amino acid substitutions) in the TD that increase binding affinity for the cognate ligand (e.g. APRIL and/or BAFF, and heterotrimers of APRIL and BAFF).

As used herein, a “B cell stimulatory receptor” refers to one or more of B cell maturation antigen (BCMA), B cell activation factor receptor (BAFF-R), and transmembrane activator and calcium modulatory and cyclophilin ligand interactor (TACI), which are related tumor necrosis factor (TNFR) superfamily receptors expressed on B cells. Engagement or ligation of these related receptors by their cognate ligands, BAFF and/or APRIL, or heterotrimers of APRIL and BAFF, regulate B cell homeostasis, including B cell survival, B cell maturation and differentiation and immunoglobulin class switching. A B cell stimulatory receptor generally contains an extracellular portion, a transmembrane domain and cytoplasmic region, in which the cytoplasmic region contains one or more TNF receptor associated factor (TRAF) binding sites. Recruitment of various TRAF molecules to the cytoplasmic domain can activate various transcription factors, such as NF-κB (e.g. NF-κB1 or NF-κB2), to mediate B cell signaling pathways regulating B cell homeostasis.

As used herein, “bind,” “bound” or grammatical variations thereof refers to the participation of a molecule in any attractive interaction with another molecule, resulting in a stable association in which the two molecules are in close proximity to one another. Binding includes, but is not limited to, non-covalent bonds, covalent bonds (such as reversible and irreversible covalent bonds), and includes interactions between molecules such as, but not limited to, proteins, nucleic acids, carbohydrates, lipids, and small molecules, such as chemical compounds including drugs.

As used herein, binding activity refer to characteristics of a molecule, e.g. a polypeptide, relating to whether or not, and how, it binds one or more binding partners. A binding activity can include any measure of binding of one molecule for a binding partner. Binding activities include the ability to bind the binding partner(s), the affinity with which it binds to the binding partner (e.g. high affinity), the avidity with which it binds to the binding partner, the strength of the bond with the binding partner and/or specificity or selectivity for binding with the binding partner.

The term “binding affinity” as used herein means the specific binding affinity of a protein for its binding partner (i.e., its counter-structure) under specific binding conditions. The binding affinity refers to the strength of the interaction between two or more molecules, such as binding partners, typically the strength of the noncovalent interactions between two binding partners. An increase or attenuation in binding affinity of an affinity-modified domain, or an immunomodulatory protein containing an affinity-modified domain, to a binding partner is determined relative to the binding affinity of the unmodified domain (e.g., the native or wild-type TD domain). Methods for determining binding affinity, or relative binding affinity, are known in art, solid-phase ELISA immunoassays, ForteBio Octet, Biacore measurements or flow cytometry. See, for example, Larsen et al., American Journal of Transplantation, vol. 5: 443-453 (2005); Linsley et al., Immunity, Vol 1 (9): 793-801 (1994). In some embodiments, binding affinity can be measured by flow cytometry, such as based on a Mean Fluorescence Intensity (MFI) in a flow binding assay.

The term “binding avidity” as used herein means the specific binding avidity, of a protein for its binding partner (i.e., its counter-structure) under specific binding conditions. In biochemical kinetics avidity refers to the accumulated strength of multiple affinities of individual non-covalent binding interactions, such as between a protein for its binding partner (i.e., its counter-structure). As such, avidity is distinct from affinity, which describes the strength of a single interaction.

The term “biological half-life” refers to the amount of time it takes for a substance, such as an immunomodulatory protein, to lose half of its pharmacologic or physiologic activity or concentration. Biological half-life can be affected by elimination, excretion, degradation (e.g., enzymatic degradation/digestion) of the substance, or absorption and concentration in certain organs or tissues of the body. In some embodiments, biological half-life can be assessed by determining the time it takes for the blood plasma concentration of the substance to reach half its steady state level (“plasma half-life”). Conjugates that can be used to derivatize and increase the biological half-life of a protein are known in the art and include, but are not limited to, multimerization domains (e.g. Fc immunoglobulin domain), polyethylene glycol (PEG), hydroxyethyl starch (HES), XTEN (extended recombinant peptides; see, WO2013130683), human serum albumin (HSA), bovine serum albumin (BSA), lipids (acylation), and poly-Pro-Ala-Ser (PAS), polyglutamic acid (glutamylation).

The term “cell surface counter-structure” (alternatively “cell surface binding partner”) as used herein is a counter-structure (alternatively is a binding partner) expressed on a mammalian cell. Typically, the cell surface binding partner is a transmembrane protein. In some embodiments, the cell surface binding partner is a receptor.

The terms “binding partner” or “counter-structure” in reference to a protein, such as a receptor, soluble ligand, or to an extracellular domain or portion thereof or affinity-modified variant thereof, refers to at least one molecule (typically a native mammalian protein) to which the referenced protein specifically binds under specific binding conditions. In some aspects an affinity-modified domain, or an immunomodulatory protein containing an affinity-modified domain, specifically binds to the binding partner of the corresponding domain of the native or wild-type protein but with increased or attenuated affinity. A “cell surface binding partner” is a binding partner expressed on a mammalian cell. Typically, the cell surface binding partner is a transmembrane protein. In some embodiments, the cell surface binding partner is a receptor, or a ligand of a receptor expressed on and by cells, such as mammalian cells, forming the immunological synapse, for example immune cells.

The term “cis” with reference to binding to cell surface molecules refers to binding to two or more different cell surface molecules, each of which is present on the surface of the same cell. In some embodiments, cis means that the two or more cell surface molecules are exclusively on one or exclusively the other (but not both) of the two mammalian cells forming the IS.

The term “conservative amino acid substitution” as used herein means an amino acid substitution in which an amino acid residue is substituted by another amino acid residue having a side chain R group with similar chemical properties (e.g., charge or hydrophobicity). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

The term, “corresponding to” with reference to positions of a protein, such as recitation that nucleotides or amino acid positions “correspond to” nucleotides or amino acid positions in a disclosed sequence, such as set forth in the Sequence Listing, refers to nucleotides or amino acid positions identified upon alignment with the disclosed sequence based on structural sequence alignment or using a standard alignment algorithm, such as the GAP algorithm. By aligning the sequences, one skilled in the art can identify corresponding residues, for example, using conserved and identical amino acid residues as guides. FIG. 9 exemplifies identification of corresponding residues by aligning two sequences.

As used herein, “domain” (typically a sequence of three or more, generally 5 or 7 or more amino acids, such as 10 to 200 amino acid residues) refers to a portion of a molecule, such as a protein or encoding nucleic acid, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as binding activity. A protein can have one, or more than one, distinct domains. For example, a domain can be identified, defined or distinguished by homology of the primary sequence or structure to related family members, such as homology to motifs. In another example, a domain can be distinguished by its function, such as an ability to interact with a biomolecule, such as a cognate binding partner. A domain independently can exhibit a biological function or activity such that the domain independently or fused to another molecule can perform an activity, such as, for example binding. A domain can be a linear sequence of amino acids or a non-linear sequence of amino acids. Many polypeptides contain a plurality of domains. Such domains are known, and can be identified by those of skill in the art. For exemplification herein, definitions are provided, but it is understood that it is well within the skill in the art to recognize particular domains by name. If needed appropriate software can be employed to identify domains. It is understood that reference to amino acids, including to a specific sequence set forth as a SEQ ID NO used to describe domain organization (e.g. of a TD domain) are for illustrative purposes and are not meant to limit the scope of the embodiments provided. It is understood that polypeptides and the description of domains thereof are theoretically derived based on homology analysis and alignments with similar molecules. Also, in some cases, adjacent N- and/or C-terminal amino acids of a given domain (e.g. TD) also can be included in a sequence, such as to ensure proper folding of the domain when expressed. Thus, the exact locus can vary, and is not necessarily the same for each protein. For example, a specific TD domain, such as specific CRD domain, can be several amino acids (1-10, such as 1, 2, 3, 4, 5, 6, 7, 8, 9 or 10 amino acids) longer or shorter.

The term “ectodomain,” “extracellular domain,” or “ECD,” which are used interchangeably herein, refers to a region of a membrane protein, such as a transmembrane protein, which lies outside the vesicular membrane (e.g., the space outside of a cell), when a full-length form of the membrane protein is expressed from a cell. For purposes herein, it is understood that reference to the ECD refers to sequences and domains that make up this region and do not require that a protein that contains an ECD is a membrane protein or that the domain is present outside a cell. For example, a soluble immunomodulatory protein can contain ECD sequences of a membrane protein fused to another moiety, such as a multimerization domain, for example an Fc region. Ectodomains often interact with specific ligands or specific cell surface receptors, such as via a binding domain that specifically binds to the ligand or cell surface receptor. Examples of binding domains include cysteine rich domains (CRDs). Ectodomains of members of the TNFR superfamily contain a TD domain (e.g. a CRD domain). Thus, reference to an ECD herein includes a full-length sequence of an ECD of a membrane protein as well as specific-binding fragments thereof containing a CRD that bind to a ligand or cognate binding partner.

The terms “effective amount” or “therapeutically effective amount” refer to a quantity and/or concentration of a therapeutic composition, such as containing an immunomodulatory protein or Fc fusion protein, that when administered ex vivo (by contact with a cell from a patient) or in vivo (by administration into a patient) either alone (i.e., as a monotherapy) or in combination with additional therapeutic agents, yields a statistically significant inhibition of disease progression as, for example, by ameliorating or eliminating symptoms and/or the cause of the disease. An effective amount for treating a disease, condition or disorder, such as an immune system disease, condition or disorder, may be an amount that relieves, lessens, or alleviates at least one symptom or biological response or effect associated with the disease, condition or disorder, prevents progression of the disease, condition or disorder, or improves physical functioning of the patient. In the case of cell therapy, the effective amount is an effective dose or number of cells administered to a patient. In some embodiments the patient is a human patient.

As used herein, a fusion protein refers to a polypeptide encoded by a nucleic acid sequence containing a coding sequence for two or more proteins, in some cases 2, 3, 4, 5 or more protein, in which the coding sequences are in the same reading frame such that when the fusion construct is transcribed and translated in a host cell, the protein is produced containing the two or more proteins. Each of the two or more proteins can be adjacent to another protein in the construct or separated by a linker polypeptide that contains, 1, 2, 3, or more, but typically fewer than 20, 15, 10, 9, 8, 7, or 6 amino acids. The protein product encoded by a fusion construct is referred to as a fusion polypeptide. An example of a fusion protein in accord with the provided embodiments is an Fc fusion protein containing an affinity-modified domain (e.g. a variant of a TACI extracellular domain or portion thereof containing a CRD) that is linked to an immunoglobulin Fc domain.

The term “half-life extending moiety” refers to a moiety of a polypeptide fusion or chemical conjugate that extends the half-life of a protein circulating in mammalian blood serum compared to the half-life of the protein that is not so conjugated to the moiety. In some embodiments, half-life is extended by greater than or about 1.2-fold, about 1.5-fold, about 2.0-fold, about 3.0-fold, about 4.0-fold, about 5.0-fold, or about 6.0-fold. In some embodiments, half-life is extended by more than 6 hours, more than 12 hours, more than 24 hours, more than 48 hours, more than 72 hours, more than 96 hours or more than 1 week after in vivo administration compared to the protein without the half-life extending moiety. The half-life refers to the amount of time it takes for the protein to lose half of its concentration, amount, or activity. Half-life can be determined for example, by using an ELISA assay or an activity assay. Exemplary half-life extending moieties include an Fc domain, a multimerization domain, polyethylene glycol (PEG), hydroxyethyl starch (HES), XTEN (extended recombinant peptides; see, WO2013130683), human serum albumin (HSA), bovine serum albumin (BSA), lipids (acylation), and poly-Pro-Ala-Ser (PAS), and polyglutamic acid (glutamylation).

An Fc (fragment crystallizable) region or domain of an immunoglobulin molecule (also termed an Fc polypeptide) corresponds largely to the constant region of the immunoglobulin heavy chain, and which, in some cases, is responsible for various functions, including the antibody's effector function(s). The Fc domain contains part or all of a hinge domain of an immunoglobulin molecule plus a CH2 and a CH3 domain. In some cases for inclusion in a provided fusion protein, all or a portion of the Fc hinge sequence may be deleted. The Fc domain can form a dimer of two polypeptide chains joined by one or more disulfide bonds. In some embodiments, the Fc is a variant Fc that exhibits reduced (e.g. reduced greater than about 30%, 40%, 50%, 60%, 70%, 80%, 90% or more) activity to facilitate an effector function. In some embodiments, reference to amino acid substitutions in an Fc region is by EU numbering system unless described with reference to a specific SEQ ID NO. EU numbering is known and is according to the most recently updated IMGT Scientific Chart (IMGT®, the international ImMunoGeneTics information ttp://www.imgt.org/IMGTScientificChart/Numbering/Hu_IGHGnber.html (created: 17 May 2001, last updated: 10 Jan. 2013) and the EU index as reported in Kabat, E. A. et al. Sequences of Proteins of Immunological interest. 5th ed. US Department of Health and Human Services, NIH publication No. 91-3242 (1991).

An immunoglobulin Fc fusion (“Fc-fusion”), such as an immunomodulatory Fc fusion protein, is a molecule comprising one or more polypeptides operably linked to an Fc region of an immunoglobulin. An Fc-fusion may comprise, for example, an Fc region operably linked to a TACI extracellular domain or portion thereof containing a CRD, including any of the provided affinity-modified variants thereof. An immunoglobulin Fc region may be linked indirectly or directly to the one or more polypeptides. Various linkers are known in the art and can optionally be used to link an Fc to a fusion partner to generate an Fc-fusion. Fc-fusions of identical species can be dimerized to form Fc-fusion homodimers. Fc fusion of non-identical species (e.g. knob into hole engineering) may be used to form Fc-fusion heterodimers. In some embodiments, the Fc is a mammalian Fc such as a murine or human Fc.

The term “host cell” refers to any cell that can be used to express a protein encoded by a recombinant expression vector. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include Chinese hamster ovary (CHO) cells or their derivatives such as Veggie CHO and related cell lines which grow in serum-free media or CHO strain DX-B11, which is deficient in DHFR.

The term “immunological synapse” or “immune synapse” (abbreviated “IS”) as used herein means the interface between a mammalian cell that expresses MHC I (major histocompatibility complex) or MHC II, such as an antigen-presenting cell or tumor cell, and a mammalian lymphocyte such as an effector T cell or Natural Killer (NK) cell.

The term “immunoglobulin” (abbreviated “Ig”) as used herein is synonymous with the term “antibody” (abbreviated “Ab”) and refers to a mammalian immunoglobulin protein including any of the five human classes: IgA (which includes subclasses IgAQ1 and IgA2), IgD, IgE, IgG (which includes subclasses IgG1, IgG2, IgG3, and IgG4), and IgM. The term is also inclusive of immunoglobulins that are less than full-length, whether wholly or partially synthetic (e.g., recombinant or chemical synthesis) or naturally produced, including any fragment thereof containing at least a portion of the variable heavy (VH) chain and/or variable light (VL) chain region of the immunoglobulin molecule that is sufficient to form an antigen binding site and, when assembled, to specifically bind antigen. The antibody also can include all or a portion of the constant region. Such fragments include antigen binding fragment (Fab), variable fragment (Fv) containing VH and VL, the single chain variable fragment (scFv) containing VH and VL linked together in one chain, as well as other antibody V region fragments, such as Fab′, F(ab)2, F(ab′)2, dsFv diabody, Fc, and Fd polypeptide fragments. Hence, it is understood that reference to an antibody herein includes full-length antibody and antigen-binding fragments. The term antibody also includes antibody compositions with polyepitopic specificity, multispecific antibodies (e.g., bispecific antibodies), diabodies, and single-chain molecules. Bispecific antibodies, homobispecific and heterobispecific, are included within the meaning of the term. Antibodies include polyclonal antibodies or monoclonal antibodies. Antibody also includes synthetic antibodies or recombinantly produced antibodies. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6.

The terms “full-length antibody,” “intact antibody” or “whole antibody” are used interchangeably to refer to an antibody in its substantially intact form, as opposed to an antibody fragment. A full-length antibody is an antibody typically having two full-length heavy chains (e.g., VH-CH1-CH2-CH3 or VH-CH1-CH2-CH3-CH4) and two full-length light chains (VL-CL) and hinge regions, such as antibodies produced from mammalian species (e.g. human, mouse, rat, rabbit, non-human primate, etc.) by antibody secreting B cells and antibodies with the same domains that are produced synthetically. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, the antigen binding and/or the variable region of the intact antibody. Antibody fragments, include, but are not limited to, Fab fragments, Fab′ fragments, F(ab′)₂ fragments, Fv fragments, disulfide-linked Fvs (dsFv), Fd fragments, Fd′ fragments; diabodies; linear antibodies (see U.S. Pat. No. 5,641,870, Example 2; Zapata et al., Protein Eng. 8(10): 1057-1062 [1995]); single-chain antibody molecules, including single-chain Fvs (scFv) or single-chain Fabs (scFab); antigen-binding fragments of any of the above and multispecific antibodies from antibody fragments.

“Fv” is composed of one heavy- and one light-chain variable region domain linked by non-covalent association. From the folding of these two domains emanate six complementarity determining regions (CDR) (3 in each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although, in some cases, at a lower affinity than the entire binding site. [0234]“dsFv” refers to an Fv with an engineered intermolecular disulfide bond, which stabilizes the V_{H}-V_{L} pair.

An “Fd fragment” is a fragment of an antibody containing a variable domain (V_{H}) and one constant region domain (C_{H}1) of an antibody heavy chain.

A “Fab fragment” is an antibody fragment that results from digestion of a full-length immunoglobulin with papain, or a fragment having the same structure that is produced synthetically, e.g., by recombinant methods. A Fab fragment contains a light chain (containing a V_{L} and C_{L}) and another chain containing a variable domain of a heavy chain (V_{H}) and one constant region domain of the heavy chain (C_{H}1).

A “F(ab′)₂ fragment” is an antibody fragment that results from digestion of an immunoglobulin with pepsin at pH 4.0-4.5, or a fragment having the same structure that is produced synthetically, e.g., by recombinant methods. The F(ab′)₂ fragment essentially contains two Fab fragments where each heavy chain portion contains an additional few amino acids including cysteine residues that form disulfide linkages joining the two fragments.

A “Fab′ fragment” is a fragment containing one half (one heavy chain and one light chain) of the F(ab′)₂ fragment.

An “Fd′ fragment” is a fragment of an antibody containing one heavy chain portion of a F(ab′)₂ fragment.

An “Fv′ fragment” is a fragment containing only the VH and VL domains of an antibody molecule.

An “scFv fragment” refers to an antibody fragment that contains a variable light chain (VL) and variable heavy chain (VH), covalently connected by a polypeptide linker in any order. The linker is of a length such that the two variable domains are bridged without substantial interference. Exemplary linkers are (Gly-Ser)₁ residues with some Glu or Lys residues dispersed throughout to increase solubility.

“Diabodies” are dimeric scFv; diabodies typically have shorter peptide linkers than scFvs, and preferentially dimerize.

The term “immunological activity” as used herein refers to one or more activities of immune cells, such as T cells or B cells, including, for example, activation, cell survival, cell proliferation, cytokine production (e.g. interferon-gamma), cytotoxicity activity, or ability to activate NF-κB pathway or other signaling cascade leading to activation of a transcription factor in the immune cell. Assays to assess immunological activity of immunomodulatory proteins can be compared to control proteins with a known activity.

An “immunomodulatory protein” or “immunomodulatory polypeptide” is a protein that modulates immunological activity. By “modulation” or “modulating” an immune response is meant that immunological activity is either enhanced or suppressed. Such modulation includes any induction, or alteration in degree or extent, or suppression of immunological activity of an immune cell, such as a B cell or a T cell. For example, soluble Fc fusion proteins herein may suppress immunological activity of B cells. An immunomodulatory protein can be a single polypeptide chain or a multimer (dimers or higher order multimers) of at least two polypeptide chains covalently bonded to each other by, for example, interchain disulfide bonds. Thus, monomeric, dimeric, and higher order multimeric proteins are within the scope of the defined term. Multimeric proteins can be homomultimeric (of identical polypeptide chains) or heteromultimeric (of different polypeptide chains).

As used herein, modification is in reference to modification of a sequence of amino acids of a polypeptide or a sequence of nucleotides in a nucleic acid molecule and includes a change in amino acids or nucleotides, respectively, of the sequence. The amino acid modification or change may be a deletion, insertion, or replacement (substitution) of amino acids or nucleotides, respectively. Methods of modifying a polypeptide are routine to those of skill in the art, such as by using recombinant DNA methodologies.

The term, a “multimerization domain” refers to a sequence of amino acids that promotes the formation of a multimer of two or more polypeptides. A multimerization domain includes sequences that promote stable interaction of a polypeptide molecule with one or more additional polypeptide molecules, each containing a complementary multimerization domain (e.g. a first multimerization domain and a second multimerization domain), which can be the same or a different multimerization domain. The interactions between complementary multimerization domains, e.g. interaction between a first multimerization domain and a second multimerization domain, form a stable protein-protein interaction to produce a multimer of the polypeptide molecule with the additional polypeptide molecule. In some cases, the multimerization domain is the same and interacts with itself to form a stable protein-protein interaction between two polypeptide chains. Generally, a polypeptide is joined directly or indirectly to the multimerization domain. Exemplary multimerization domains include the immunoglobulin sequences or portions thereof, leucine zippers, hydrophobic regions, hydrophilic regions, and compatible protein-protein interaction domains. The multimerization domain, for example, can be an immunoglobulin constant region or domain, such as, for example, the Fc domain or portions thereof from IgG, including IgG1, IgG2, IgG3 or IgG4 subtypes, IgA, IgE, IgD and IgM and modified forms thereof.

The terms “nucleic acid” and “polynucleotide” are used interchangeably to refer to a polymer of nucleic acid residues (e.g., deoxyribonucleotides or ribonucleotides) in either single- or double-stranded form. Unless specifically limited, the terms encompass nucleic acids containing known analogues of natural nucleotides and that have similar binding properties to it and are metabolized in a manner similar to naturally-occurring nucleotides. Unless otherwise indicated, a particular nucleic acid sequence also implicitly encompasses conservatively modified variants thereof (e.g., degenerate codon substitutions) and complementary nucleotide sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid or polynucleotide encompasses cDNA or mRNA encoded by a gene.

The terms “in operable combination,” “in operable order” and “operably linked” as used herein refer to the linkage of nucleic acid sequences in such a manner or orientation that the segments are arranged so that they function in concert for their intended purposes. In some embodiments, the term refers to linkage of nucleic acids to produce a nucleic acid molecule capable of directing the transcription of a given gene and/or to produce a desired protein molecule that is functional. For example, segments of a DNA sequence, e.g. a coding sequence and a regulatory sequence(s), are linked in such a way as to permit gene expression when the appropriate molecules (e.g. transcriptional activator proteins) are bound to the regulatory sequence.

The term “pharmaceutical composition” refers to a composition suitable for pharmaceutical use in a mammalian subject, often a human. A pharmaceutical composition typically comprises an effective amount of an active agent (e.g., an immunomodulatory protein) and a carrier, excipient, or diluent. The carrier, excipient, or diluent is typically a pharmaceutically acceptable carrier, excipient or diluent, respectively.

The terms “polypeptide” and “protein” are used interchangeably herein and refer to a molecular chain of two or more amino acids linked through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. The terms also include molecules in which one or more amino acid analogs or non-canonical or unnatural amino acids are included as can be synthesized or expressed recombinantly using known protein engineering techniques. In addition, proteins can be derivatized as described herein by well-known organic chemistry techniques.

The term “purified” as applied to nucleic acids, such as encoding immunomodulatory proteins, or proteins (e.g. immunomodulatory proteins) generally denotes a nucleic acid or polypeptide that is substantially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or protein is at least about 50% pure, usually at least about 75%, 80%, 85%, 90%, 95%, 96%, 99% or more pure (e.g., percent by weight or on a molar basis).

The term “recombinant” indicates that the material (e.g., a nucleic acid or a polypeptide) has been artificially (i.e., non-naturally) altered by human intervention. The alteration can be performed on the material within, or removed from, its natural environment or state. For example, a “recombinant nucleic acid” is one that is made by recombining nucleic acids, e.g., during cloning, affinity modification, DNA shuffling or other well-known molecular biological procedures. A “recombinant DNA molecule,” is comprised of segments of DNA joined together by means of such molecular biological techniques. The term “recombinant protein” or “recombinant polypeptide” as used herein refers to a protein molecule (e.g., an immunomodulatory protein) which is expressed using a recombinant DNA molecule. A “recombinant host cell” is a cell that contains and/or expresses a recombinant nucleic acid or that is otherwise altered by genetic engineering, such as by introducing into the cell a nucleic acid molecule encoding a recombinant protein, such as an immunomodulatory protein provided herein. Transcriptional control signals in eukaryotes comprise “promoter” and “enhancer” elements. Promoters and enhancers consist of short arrays of DNA sequences that interact specifically with cellular proteins involved in transcription. Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest.

The term “recombinant expression vector” as used herein refers to a DNA molecule containing a desired coding sequence (e.g., encoding an immunomodulatory protein) and appropriate nucleic acid sequences necessary for the expression of an operably linked coding sequence in a particular cell. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the recombinant expression vector, operably linked to the coding sequence so that the expressed protein can be secreted by the recombinant host cell, such as for its expression as a secretable protein or for more facile isolation or purification of the immunomodulatory protein from the cell, if desired. The term includes the vector as a self-replicating nucleic acid structure as well as the vector incorporated into the genome of a host cell into which it has been introduced. Among the vectors are viral vectors, such as lentiviral vectors.

The term “sequence identity” as used herein refers to the sequence identity between genes or proteins at the nucleotide or amino acid level, respectively. “Sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. Methods for the alignment of sequences for comparison are well known in the art, such methods include GAP, BESTFIT, BLAST, BLAST-2, ALIGN or Megalign (DNASTAR) software, FASTA and TFASTA. The BLAST algorithm calculates percent sequence identity and performs a statistical analysis of the similarity between the two sequences. The software for performing BLAST analysis is publicly available through the National Center for Biotechnology Information (NCBI) website. In some cases, a percent sequence identity can be determined as the percentage of amino acid residues (or nucleotide residues) in a candidate sequence that are identical with the amino acid residues (or nucleotide residues) in a reference sequence, after aligning the sequences and introducing gaps, if necessary, to achieve the maximum percent sequence identity. Reference to sequence identity includes sequence identity across the full length of each of the sequences being compared. Those skilled in the art can determine appropriate parameters for aligning sequences, including any algorithms needed to achieve maximal alignment over the full length of the sequences being compared.

The term “soluble” as used herein in reference to proteins means that the protein is not a membrane protein or is not anchored in a cell membrane. A protein can be constructed as a soluble protein by inclusion of only an extracellular domain or a portion thereof and without a transmembrane domain. In some cases, solubility of a protein can be improved by linkage or attachment, directly or indirectly via a linker, to an Fc domain or other half-life extending molecule, which, in some cases, also can improve the stability and/or half-life of the protein. In some aspects, a soluble protein is an Fc fusion protein.

The term “specifically binds” as used herein means the ability of a protein, under specific binding conditions, to bind to a target protein such that its affinity or avidity is at least 10 times as great, but optionally 50, 100, 250 or 500 times as great, or even at least 1000 times as great as the average affinity or avidity of the same protein to a collection of random peptides or polypeptides of sufficient statistical size. A specifically binding protein need not bind exclusively to a single target molecule but may specifically bind to more than one target molecule. In some cases, a specifically binding protein may bind to a protein that has similarity in structural conformation with the target protein (e.g., paralogs or orthologs). Those of skill will recognize that specific binding to a molecule having the same function in a different species of animal (i.e., ortholog) or to a molecule having a substantially similar epitope as the target molecule (e.g., paralog) is possible and does not detract from the specificity of binding which is determined relative to a statistically valid collection of unique non-targets (e.g., random polypeptides). Thus, an immunomodulatory protein of the invention may specifically bind to more than one distinct species of target molecule due to cross-reactivity. Solid-phase ELISA immunoassays, ForteBio Octet or Biacore measurements can be used to determine specific binding between two proteins. Generally, interactions between two binding proteins have dissociation constants (Kd) less than about 1×10⁻⁵ M, and often as low as about 1×10⁻¹² M. In certain aspects of the present disclosure, interactions between two binding proteins have dissociation constants of less than about 1×10⁻⁶ M, 1×10⁻⁷ M, 1×10⁻⁸ M, 1×10⁻⁹ M, 1×10⁻¹⁰ M, or 1×10⁻¹¹ M or less.

The term “specific binding fragment” or “fragment” as used herein in reference to a protein means a polypeptide that is shorter than a full-length protein or a specific domain or region thereof and that specifically binds in vitro and/or in vivo to a binding partner of the full-length protein or of the specific domain or region. A specific finding fragment is in reference to a fragment of a full-length extracellular domain of a polypeptide or a binding domain of a polypeptide, but that still binds to a binding partner of the binding domain. For example, a specific binding fragment is in reference to a fragment of an extracellular domain of a full-length TNFR family member or a full-length TNFR domain (TD) thereof (e.g. CRD), but that still binds to a binding partner of the TNFR family member or of a CRD of an TNFR family member. In some embodiments, the specific binding fragment is at least about 20%, 30%, 40%, 50%, 60%, 70%, 75%, 80%, 85%, 90%, 95%, 96%, 97%, 98%, or 99% the sequence length of the full-length sequence of the extracellular domain or of a domain or region of the extracellular domain. In some embodiments, the specific binding fragment can have an amino acid length of at least 50 amino acids, such as at least 60, 70, 80, 90, 100, or 110 amino acids. In some embodiments, the specific binding fragment includes the CRD1 and/or CRD2 domain. In some embodiments, the specific binding fragment includes the CRD2 domain.

As used herein, a “subject” is a mammal, such as a human or other animal, and typically is human. The subject can be male or female and can be any suitable age, including infant, juvenile, adolescent, adult, and geriatric subjects.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods.

The term “TNF receptor superfamily” or “TNFRSF” as used herein means the group of cell surface cytokine receptors that are all type I (N-terminus extracellular) transmembrane glycoproteins that contain one to six cysteine rich domains (CRD) in their extracellular domain. Molecules are categorized as members of this superfamily based on the shared structural features that include the one or more cysteine rich domain (CRD) present in their N-terminal extracellular region, which often play a role in protein binding of their cognate binding partner or ligand. A TNFRSF protein may have only one or several CRDs (e.g. CRD1, CRD2, etc.). Typically, ECD or ectodomain of TNFRSF members contain between 1 and 6 pseudorepeats of CRDs. For example, BAFF-receptor and BCMA each contain one CRD while TACI contains two CRDs (CRD1 and CRD2). TNFRSF members are usually trimeric or multimeric complexes that are stabilized by their intracysteine disulfide bonds. Binding of TNFRSF proteins to their ligands facilitates various biological activities in cells, such as the induction of apoptotic cell death or cell survival and proliferation.

The term “TD” refers to a structural domain or domains of TNFRSF proteins or of TNF family ligands. For example, a TD of a TNFRSF protein is a cysteine-rich domain (CRD) module of about 40 amino acids containing six (6) conserved cysteines. Hence, reference to CRD also can be used interchangeably with the term TD in reference to a TD of a TNFRSF protein. The six cysteines are involved in formation of intrachain disulphide bonds. The extracellular domain (ECD) of TNFRSF members contains one or more CRD domains; hence, the term TD is also used with reference to the ECD of such protein molecules. Reference to a variant TD (vTD) refers to a variant or modified sequence of a TD.

The term “trans” with reference to binding to cell surface molecules refers to binding to two different cell surface molecules, each of which is present on the surface of a different cell. In some embodiments, trans means that with respect to two different cell surface molecules, the first is exclusively present on one of the two mammalian cells forming the IS and the second is present exclusively on the second of the two mammalian cells forming the IS.

The term “transmembrane protein” as used herein means a membrane protein that substantially or completely spans a lipid bilayer such as those lipid bilayers found in a biological membrane such as a mammalian cell, or in an artificial construct such as a liposome. The transmembrane protein comprises a transmembrane domain (“transmembrane domain”) by which it is integrated into the lipid bilayer and by which the integration is thermodynamically stable under physiological conditions. Transmembrane domains are generally predictable from their amino acid sequence via any number of commercially available bioinformatics software applications on the basis of their elevated hydrophobicity relative to regions of the protein that interact with aqueous environments (e.g., cytosol, extracellular fluid). A transmembrane domain is often a hydrophobic alpha helix that spans the membrane. A transmembrane protein can pass through both layers of the lipid bilayer once or multiple times.

The terms “treating,” “treatment,” or “therapy” of a disease, condition or disorder as used herein mean slowing, stopping or reversing the disease or disorders progression, as evidenced by decreasing, cessation or elimination of either clinical or diagnostic symptoms, by administration of an immunomodulatory protein or engineered cells of the present invention either alone or in combination with another compound as described herein. “Treating,” “treatment,” or “therapy” also means a decrease in the severity of symptoms in an acute or chronic disease, condition or disorder or a decrease in the relapse rate as for example in the case of a relapsing or remitting autoimmune disease course or inflammatory condition or a decrease in inflammation in the case of an inflammatory aspect of an autoimmune disease or inflammatory condition. “Preventing,” “prophylaxis,” or “prevention” of a disease, condition or disorder as used in the context of this invention refers to the administration of an immunomodulatory protein of the present invention, either alone or in combination with another compound, to prevent the occurrence or onset of a disease, condition or disorder or some or all of the symptoms of a disease, condition or disorder or to lessen the likelihood of the onset of a disease, condition or disorder.

The term “variant” (also “modified” or mutant,” which can be used interchangeably) as used in reference to a variant protein or polypeptide means a protein, such as a mammalian (e.g., human or murine) protein created by human intervention. The variant is a polypeptide having an altered or modified amino acid sequence, such as by one or more amino acid substitutions, deletions, additions or combinations thereof, relative to an unmodified or wild-type protein or to a domain thereof. A variant polypeptide can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences, such as amino acid substitutions. A variant polypeptide generally exhibits at least about 50%, 60%, 70%, 80%, 85%, 86%, 87%, 88%, 89%, 90%, 91%, 92%, 93%, 94%, 95%, 96%, 97%, 98%, 99% or more sequence identity to a corresponding form of a wild-type or unmodified protein, such as a mature sequence thereof (lacking the signal sequence) or a portion thereof containing the extracellular domain or an binding domain thereof. Non-naturally occurring amino acids as well as naturally occurring amino acids are included within the scope of permissible substitutions or additions. A variant protein is not limited to any particular method of making and includes, for example, chemical synthesis, recombinant DNA techniques, or combinations thereof. A variant protein of the invention specifically binds to at least one or more binding partners. In some embodiments, the altered amino acid sequence results in an altered (i.e., increased or decreased) binding activity, such as binding affinity or avidity, to the one or more binding partners. A variant protein may thus be an “affinity-modified” protein as described herein.

The term “wild-type” or “natural” or “native,” which are used interchangeably, as used herein is used in connection with biological materials such as nucleic acid molecules, proteins, host cells, and the like, which are found in nature and not modified by human intervention.

### II. TACI IMMUNOMODULATORY PROTEINS AND VARIANT TACI POLYPEPTIDES

Provided herein are TACI immunomodulatory proteins that contain a portion of the extracellular domain (ECD) of the TACI receptor, or a variant thereof, that bind to at least one TACI cognate binding partner. Also provided herein are variant TACI polypeptides that exhibit altered (e.g. increased) binding activity or affinity for one or more of a TACI cognate binding partner. In some embodiments, the TACI cognate binding partner is one or more of BAFF or APRIL or is a BAFF/APRIL heterotrimer. The provided TACI immunomodulatory proteins and polypeptides include soluble fusion proteins thereof in which the TACI portion of the extracellular domain or variant thereof is linked to another moiety, such as an immunoglobulin Fc or other multimerization domain or half-life extending moiety. Thus, in some embodiments the immunomodulatory protein is a TACI-Fc fusion protein. In some embodiments, provided is a TACI-Fc fusion protein containing (1) a TACI polypeptide composed of the extracellular domain of the TACI receptor or a portion thereof, or a variant TACI polypeptide thereof, that binds to at least one TACI cognate binding partner, and (2) an Fc domain. The TACI polypeptide or variant TACI polypeptide can be linked directly or indirectly (e.g. via a peptide linker) to the Fc domain.

TACI is a tumor necrosis factor receptor family member characterized by having an extracellular domain (ECD) containing cysteine-rich pseudo-repeat domains (CRDs). TACI is a membrane bound receptor, which has an extracellular domain containing two cysteine-rich pseudo-repeats (CRD1 and CRD2), a transmembrane domain and a cytoplasmic domain that interacts with CAML (calcium-modulator and cyclophilin ligand), an integral membrane protein located at intracellular vesicles which is a co-inducer of NF-AT activation when overexpressed in Jurkat cells. TACI is associated with B cells and a subset of T cells. The TACI receptor binds two members of the tumor necrosis factor (TNF) ligand family. One ligand is designated BAFF (B cell Activating Factor of the TNF Family), and also is variously designated as ZTNF4, “neutrokine-α,” “BLyS,” “TALL-1,” and “THANK” (Yu et al., international publication No. WO98/18921 (1998), Moore et al., Science 285:269 (1999); Mukhopadhyay et al., J. Biol. Chem. 274:15978 (1999); Schneider et al., J. Exp. Med. 189:1747 (1999); Shu et al., J. Leukoc. Biol. 65:680 (1999)). The other ligand has been designated as APRIL, and also is variously designated as “ZTNF2” and “TNRF death ligand-1” (Hahne et al., J. Exp. Med. 188:1185 (1998); Kelly et al., Cancer Res. 60:1021 (2000)). Both ligands are also bound by the B-cell maturation receptor (BCMA) (Gross et al., Nature 404:995 (2000)). Binding of TACI receptor to its ligands BAFF or APRIL stimulates B cell responses, including T cell-independent B cell antibody responses, isotype switching, and B cell homeostasis.

The amino acid sequence of full-length TACI is set forth in SEQ ID NO:88. The protein is a type III membrane protein and lacks a signal peptide; following expression in eukaryotic cells the N-terminal methionine is removed. In some embodiments, a mature TACI protein does not contain the N-terminal methionine as set forth in SEQ ID NO:88. The extracellular domain of TACI (amino acid residues 1-166 of SEQ ID NO:88; ECD set forth in SEQ ID NO:122) contains two cysteine rich domain (CRDs, hereinafter also called a tumor necrosis family receptor domain or TD), each of which exhibit affinity for binding to BAFF and APRIL. The first cysteine rich domain (CRD1) contains amino acid residues 34-66 of the sequence set forth in SEQ ID NO:122. The second cysteine rich domain (CRD2) corresponds to amino acids 71-104 of the sequence set forth in SEQ ID NO:122. TACI also contains a stalk region of about 60 amino acids following the second cysteine repeat in the extracellular domain, corresponding to amino acid residues 105-165 of the sequence set forth in SEQ ID NO:122.

In some embodiments, the variant TACI polypeptides provided herein contain one or more amino acid modifications, such as one or more substitutions (alternatively, “mutations” or “replacements”), deletions or additions in the extracellular domain of a reference TACI polypeptide, such as a wild-type or unmodified TACI polypeptide containing a CRD(s) (hereinafter also called TDs). Thus, a provided variant TACI polypeptide is or comprises a variant TD (“vTD”) in which the one or more amino acid modifications (e.g. substitutions) is in a CRD. In some embodiments, the one or more amino acids modifications, such as one or more substitutions (alternatively, “mutations” or “replacements”), deletions or additions, is in the CRD1 region. In some embodiments, the one or more amino acids modifications, such as one or more substitutions (alternatively, “mutations” or “replacements”), deletions or additions, is in the CRD2 region. In some embodiments, the one or more amino acids modifications, such as one or more substitutions (alternatively, “mutations” or “replacements”), deletions or additions, is in amino acids within both the CRD1 and CRD2 regions.

In some embodiments, the reference (e.g. unmodified) TACI sequence is a wild-type TACI sequence or is a portion thereof that contains one or both CRDs. In some embodiments, the reference (e.g., unmodified) TACI is or comprises the extracellular domain (ECD) of TACI or a portion thereof containing one or both CRD domains. In some embodiments, the extracellular domain of a reference (e.g., unmodified) TACI polypeptide comprises a CRD1 and CRD2. However, the variant TACI polypeptide need not comprise both the CRD1 and the CRD2. In some embodiments, the variant TACI polypeptide comprises or consists essentially of the CRD1 or a specific binding fragment thereof. In some embodiments, the variant TACI polypeptide comprises or consists essentially of the CRD2 or specific binding fragments thereof. In some embodiments, the variant TACI is a soluble polypeptide and lacks a transmembrane domain. In some embodiments, the variant TACI polypeptide further comprises a transmembrane domain and, in some cases, also a cytoplasmic domain.

In some embodiments, the reference (e.g., unmodified) TACI sequence is a mammalian TACI sequence. In some embodiments, the reference (e.g., unmodified) TACI sequence can be a mammalian TACI that includes, but is not limited to, human, mouse, cynomolgus monkey, or rat. In some embodiments, the reference (e.g., unmodified) TACI sequence is human. The extracellular domain of an exemplary human TACI sequence is set forth in SEQ ID NO:122.

In some embodiments, the reference (e.g., unmodified) TACI sequence has (i) the sequence of amino acids set forth in SEQ ID NO:122 or a sequence thereof that lacks the N-terminal methionine, (ii) a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO:122 and that binds to APRIL, BAFF or an APRIL/BAFF heterotrimer, or (iii) is a fragment or portion of (i) or (ii) containing a CRD1 and/or CRD2, in which the portion binds to APRIL, BAFF or an APRIL/BAFF heterotrimer. In some embodiments, the reference (e.g., unmodified) TACI sequence lacks the N-terminal methionine as set forth in SEQ ID NO: 122.

TACI Extracellular Domain (ECD):
SEQ ID NO: 122
MSGLGRSRRGGRSRVDQEERFPQGLWTGVAMRSCPEEQYWDPLLGTCMSC
KTICNHQSQRTCAAFCRSLSCRKEQGKFYDHLLRDCISCASICGQHPKQC
AYFCENKLRSPVNLPPELRRQRSGEVENNSDNSGRYQGLEHRGSEASPAL
PGLKLSADQVALVYST

In some embodiments, the reference (e.g. unmodified) TACI sequence is an extracellular domain sequence of TACI that is a portion of the ECD that contains an N-terminal deletion relative to the sequence of amino acids set forth in SEQ ID NO:122. In some embodiments, the N-terminal deletion is deletion of N-terminal amino acid residues 1-28 corresponding to residues set forth in SEQ ID NO:122. In some embodiments, the N-terminal deletion is deletion of N-terminal amino acid residues 1-29 corresponding to residues set forth in SEQ ID NO:122. In some embodiments, the N-terminal deletion is deletion of N-terminal amino acid residues 1-30 corresponding to residues set forth in SEQ ID NO:122. In some embodiments, the N-terminal deletion is deletion of N-terminal amino acid residues 1-31 corresponding to residues set forth in SEQ ID NO:122. In some embodiments, the N-terminal deletion is deletion of N-terminal amino acid residues 1-32 corresponding to residues set forth in SEQ ID NO:122. In some embodiments, the N-terminal deletion is deletion of N-terminal amino acid residues 1-33 corresponding to residues set forth in SEQ ID NO:122.

In embodiments of any of the provided embodiments, the reference (e.g. unmodified) TACI sequence is an ECD portion that contains deletion of one or more residues of the stalk portion of the TACI extracellular domain. In some embodiments, the reference (e.g. unmodified) TACI sequence is an ECD portion that lacks one or more contiguous C-terminal amino acid residues beginning at residue 105 and up to or including amino acid residue 166 corresponding to residues of the ECD sequence set forth in SEQ ID NO:122. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61 or 62 of the ECD sequence is deleted.

In some embodiments, the reference (e.g. unmodified) TACI sequence contains an ECD portion having a contiguous sequence of amino acids that includes the CRD1 and/or CRD2 (e.g. CRD1 and CRD2 or CRD2 only) and only a segment or portion of the stalk sequence. Suitable stalk segments include one or more amino acids of amino acid residues 105 to 154 of SEQ ID NO:122. For example, the stalk segment can consist of the following with reference to SEQ ID NO:122: amino acid residue 105, amino acid residues 105 to 106, amino acid residues 105 to 107, amino acid residues 105 to 108, amino acid residues 105 to 109, amino acid residues 105 to 110, amino acid residues 105 to 111, amino acid residues 105 to 112, amino acid residues 105 to 113, amino acid residues 105 to 114, amino acid residues 105 to 115, amino acid residues 105 to 116, amino acid residues 105 to 117, amino acid residues 105 to 118, amino acid residues 105 to 119, amino acid residues 105 to 120, amino acid residues 105 to 121, amino acid residues 105 to 122, amino acid residues 105 to 123, amino acid residues 105 to 124, amino acid residues 105 to 125, amino acid residues 105 to 126, amino acid residues 105 to 127, amino acid residues 105 to 128, amino acid residues 105 to 129, amino acid residues 105 to 130, amino acid residues 105 to 131, amino acid residues 105 to 132, amino acid residues 105 to 133, amino acid residues 105 to 134, amino acid residues 105 to 135, amino acid residues 105 to 136, amino acid residues 105 to 137, amino acid residues 105 to 138, amino acid residues 105 to 139, amino acid residues 105 to 140, amino acid residues 105 to 141, amino acid residues 105 to 142, amino acid residues 105 to 143, amino acid residues 105 to 144, amino acid residues 105 to 145, amino acid residues 105 to 146, amino acid residues 105 to 147, amino acid residues 105 to 148, amino acid residues 105 to 149, amino acid residues 105 to 150, amino acid residues 105 to 151, amino acid residues 105 to 152, amino acid residues 105 to 153, and amino acid residues 105 to 154.

In some embodiments, the reference (e.g. unmodified) TACI sequence lacks or is mutated in one or more potential furin cleavage sites. In some cases, the reference (e.g. unmodified) TACI sequence is an ECD or portion that in which the arginine residue at position 119 is mutated, e.g. R119G. In some cases, the reference (e.g. unmodified) TACI sequence is an ECD or portion that in which the glutamine residue at position 121 is mutated, e.g. Q121P. In some cases, the reference (e.g. unmodified) TACI sequence is an ECD or portion that in which the arginine residue at position 122 is mutated, e.g. R122Q.

In some embodiments, the reference TACI sequence is a TACI ECD sequence as set forth in international PCT publication No. WO2000/067034, WO2002/094852 or WO2008/154814.

In some embodiments, the reference TACI sequence is a TACI ECD sequence that has or consists of the sequence set forth in SEQ ID NO:131.

TACI ECD (CRD1/CRD2):
SEQ ID NO: 131
SRVDQEER FPQGLWTGVA MRSCPEEQYW DPLLGTCMSCKTICNHQSQ
R TCAAFCRSLS CRKEQGKFYD HLLRDCISCA SICGQHPKQCAYFCE
NKLRSPVNLPPEL

In some embodiments, the reference TACI sequence is a TACI ECD sequence that has or consists of the sequence set forth in SEQ ID NO:130.

TACI ECD (CRD1/CRD2):
SEQ ID NO: 130
AMRSCPEEQYWDPLLGTCMSCKTICNHQSQRTCAAFCRSLSCRKEQGKFY
DHLLRDCISCASICGQHPKQCAYFCENKLRS

In some embodiments, the reference TACI sequence is a TACI ECD sequence that has or consists of the sequence set forth in SEQ ID NO:1 (encoded by the sequence of nucleotides set forth in SEQ ID NO:36).

TACI ECD (CRD1/CRD2):
SEQ ID NO: 1
VAMRSCPEEQYWDPLLGTCMSCKTICNHQSQRTCAAFCRSLSCRKEQGKF
YDHLLRDCISCASICGQHPKQCAYFCENKLRS

In some embodiments, the reference TACI sequence is an extracellular domain region of TACI that consists essentially of only the CRD2 sequence and that is deleted in or lacks the entirety of the sequence of the CRD1 and substantially all of the stalk region. Although previous studies have shown that residues in the stalk region may contain a protease cleavage site, it was believed that at least the CRD1 and CRD2 was required for sufficient expression and/or binding activity of TACI for its cognate ligands. For example, international PCT publication No. WO2002/094852 demonstrated that a TACI molecule containing a CRD1 and CRD2, but in which the whole amino terminal region and a partial sequence of the stalk region was deleted, exhibited reduced protein degradation when expressed. Other studies showed that at least a portion of the N-terminal region before the CRD1 was necessary for sufficient binding activity of TACI for its cognate ligands, see e.g. international publication No. WO2008/154814, in which residues 13-118 or 13-108 of the TACI extracellular region were determined to be necessary for biological activity while minimizing degradation of TACI during expression. Surprisingly, it is found herein (e.g. Example 3) that a TACI extracellular region that consists essentially only of the CRD2 with a small portion of the stalk region exhibits substantially improved cognate binding activity compared to a longer TACI molecule containing both the CRD1 and CRD2.

Provided herein is an immunomodulatory protein (e.g. TACI-Fc fusion protein) containing a TACI polypeptide that is a portion of the TACI extracellular domain (ECD) region that contains the CRD2, with a deletion of the N-terminal region and CRD1 and deletion of one or more residues of the stalk portion of the TACI extracellular domain, e.g. relative to the sequence of amino acids set forth in SEQ ID NO:122. In some embodiments, the portion of the TACI extracellular domain that contains the CRD2 includes amino acid residues 71-104 corresponding to residues set forth in SEQ ID NO:122. In provided embodiments, the TACI polypeptide of the immunomodulatory protein contains deletion of N-terminal amino acid residues 1-66 corresponding to residues set forth in SEQ ID NO:122. In provided embodiments, the TACI polypeptide of the immunomodulatory protein contains deletion of N-terminal amino acid residues 1-67 corresponding to residues set forth in SEQ ID NO:122. In provided embodiments, the TACI polypeptide of the immunomodulatory protein contains deletion of N-terminal amino acid residues 1-68 corresponding to residues set forth in SEQ ID NO:122. In provided embodiments, the TACI polypeptide of the immunomodulatory protein contains deletion of N-terminal amino acid residues 1-69 corresponding to residues set forth in SEQ ID NO:122. In provided embodiments, the TACI polypeptide of the immunomodulatory protein contains deletion of N-terminal amino acid residues 1-70 corresponding to residues set forth in SEQ ID NO:122. In embodiments of any such embodiments, the TACI polypeptide of the immunomodulatory protein lacks one or more contiguous C-terminal amino acid residues beginning at residue 105 and up to or including amino acid residue 166 corresponding to residues of the ECD sequence set forth in SEQ ID NO:122. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61 or 62 of the ECD sequence is deleted.

In some embodiments, an immunomodulatory protein (e.g. TACI-Fc fusion protein) provided herein has a TACI polypeptide with a sequence that contains an ECD portion having a contiguous sequence of amino acids of a TACI ECD that includes the CRD2 (e.g. residues 71-104 with reference to SEQ ID NO:122), but with a deletion of the N-terminal region and CRD1 and deletion of one or more residues of the stalk portion of the TACI extracellular domain, e.g. relative to the sequence of amino acids set forth in SEQ ID NO:122. For example, the TACI ECD portion can consist of the following with reference to amino acid residues set forth in SEQ ID NO:122: amino acid residues 67 to 118, amino acid residues 67 to 117, amino acid residues 67 to 116, amino acid residues 67 to 115, amino acid residues 67 to 114, amino acid residues 67 to 113, amino acid residues 67 to 112, amino acid residues 67 to 111, amino acid residues 67 to 110, amino acid residues 67 to 109, amino acid residues 67 to 108, amino acid residues 67 to 107, amino acid residues 67 to 106, amino acid residues 67 to 105, or amino acid residues 67 to 104. In some examples, the TACI ECD portion can consist of the following with reference to residues set forth in SEQ ID NO: 122: amino acid residues 68 to 118, amino acid residues 68 to 117, amino acid residues 68 to 116, amino acid residues 68 to 115, amino acid residues 68 to 114, amino acid residues 68 to 113, amino acid residues 68 to 112, amino acid residues 68 to 111, amino acid residues 68 to 110, amino acid residues 68 to 109, amino acid residues 68 to 108, amino acid residues 68 to 107, amino acid residues 68 to 106, amino acid residues 68 to 105, or amino acid residues 68 to 104. In some examples, the TACI ECD portion can consist of the following with reference to residues set forth in SEQ ID NO: 122: amino acid residues 69 to 118, amino acid residues 69 to 117, amino acid residues 69 to 116, amino acid residues 69 to 115, amino acid residues 69 to 114, amino acid residues 69 to 113, amino acid residues 69 to 112, amino acid residues 69 to 111, amino acid residues 69 to 110, amino acid residues 69 to 109, amino acid residues 69 to 108, amino acid residues 69 to 107, amino acid residues 69 to 106, amino acid residues 69 to 105, or amino acid residues 69 to 104. In some examples, the TACI ECD portion can consist of the following with reference to residues set forth in SEQ ID NO: 122: amino acid residues 70 to 118, amino acid residues 70 to 117, amino acid residues 70 to 116, amino acid residues 70 to 115, amino acid residues 70 to 114, amino acid residues 70 to 113, amino acid residues 70 to 112, amino acid residues 70 to 111, amino acid residues 70 to 110, amino acid residues 70 to 109, amino acid residues 70 to 108, amino acid residues 70 to 107, amino acid residues 70 to 106, amino acid residues 70 to 105, or amino acid residues 70 to 104. In some examples, the TACI ECD portion can consist of the following with reference to residues set forth in SEQ ID NO: 122: amino acid residues 71 to 118, amino acid residues 71 to 117, amino acid residues 71 to 116, amino acid residues 71 to 115, amino acid residues 71 to 114, amino acid residues 71 to 113, amino acid residues 71 to 112, amino acid residues 71 to 111, amino acid residues 71 to 110, amino acid residues 71 to 109, amino acid residues 71 to 108, amino acid residues 71 to 107, amino acid residues 71 to 106, amino acid residues 71 to 105, or amino acid residues 71 to 104. Any of the above TACI ECD sequences also can be a TACI reference sequence in accord with the immunomodulatory proteins provided herein, in which such immunomodulatory proteins contain a variant TACI polypeptide that is modified by one or more amino acid modification (e.g. substitution) as described herein compared to such TACI reference sequence.

In particular, among TACI polypeptides provided herein is a TACI ECD sequence that has or consists of the sequence set forth in SEQ ID NO:13 (encoded by the sequence of nucleotides set forth in SEQ ID NO:48). In some embodiments, the reference TACI sequence has or consists of the sequence set forth in SEQ ID NO:13, in which a provided variant TACI polypeptide is modified by one or more amino acid modification (e.g. substitution) as described herein compared to such reference TACI sequence.

TACI ECD sequence (CRD2):
SEQ ID NO: 13
SLSCRKEQGKFYDHLLRDCISCASICGQHPKQCAYFCENKLRS

In some embodiments, the reference TACI sequence comprises the amino acid sequence set forth in SEQ ID NO:204. In some embodiments, the reference TACI sequence consists of the amino acid sequence set forth in SEQ ID NO:204. In some embodiments, the reference TACI sequence comprises the amino acid sequence set forth in SEQ ID NO:206. In some embodiments, the reference TACI sequence consists of the amino acid sequence set forth in SEQ ID NO:206. In some embodiments, the reference TACI sequence comprises the amino acid sequence set forth in SEQ ID NO:215. In some embodiments, the reference TACI sequence consists of the amino acid sequence set forth in SEQ ID NO:215. In some embodiments, the reference TACI sequence comprises the amino acid sequence set forth in SEQ ID NO:217. In some embodiments, the reference TACI sequence consists of the amino acid sequence set forth in SEQ ID NO:217. In some embodiments, the reference TACI sequence comprises the amino acid sequence set forth in SEQ ID NO:240. In some embodiments, the reference TACI sequence consists of the amino acid sequence set forth in SEQ ID NO:240. In some embodiments, the reference TACI sequence comprises the amino acid sequence set forth in SEQ ID NO:241. In some embodiments, the reference TACI sequence consists of the amino acid sequence set forth in SEQ ID NO: 241.

Among provided TACI polypeptides are variant TACI polypeptides. Also provided are immunomodulatory proteins, such as TACI-Fc fusion proteins, which contain a provided variant TACI polypeptide. In embodiments of any of the provided embodiments, the variant TACI sequence has the sequence of the reference (e.g. unmodified) TACI sequence, such as any described above, but additionally contains one more amino acid modifications, such as one or more amino acid substitutions. In particular, provided herein are variant TACI polypeptides containing at least one affinity-modified TD domain (e.g., CRD1 and/or CRD2) or a specific binding fragment thereof that contains one or more amino acid substitutions in a TD domain of a reference (e.g., unmodified or wild-type) TACI polypeptide, such that the variant TACI polypeptide exhibits altered (e.g. increased) binding activity or affinity for one or both of APRIL or BAFF compared to the reference (e.g., unmodified or wild-type) TACI polypeptide. In some embodiments, a variant TACI polypeptide has a binding affinity for APRIL and/or BAFF that differs from that of a reference (e.g., unmodified or wild-type) TACI polypeptide control sequence as determined by, for example, solid-phase ELISA immunoassays, flow cytometry or Biacore assays. Binding affinities for each of the cognate binding partners are independent; that is, in some embodiments, a variant TACI polypeptide has an increased binding affinity for one or both APRIL and BAFF, and a decreased or unchanged binding affinity for the other of APRIL or BAFF, relative to a reference (e.g., unmodified or wild-type) TACI polypeptide.

In some embodiments, the variant TACI polypeptide has an increased binding affinity for BAFF, relative to the reference (unmodified or wild-type) TACI polypeptide. In some embodiments, the variant TACI polypeptide has an increased binding affinity for APRIL relative to the reference (unmodified or wild-type) TACI polypeptide. In some embodiments, the variant TACI polypeptide has an increased binding affinity for APRIL and BAFF relative to the reference (unmodified or wild-type) TACI polypeptide. The cognate ligands BAFF and/or APRIL can be a mammalian protein, such as a human protein or a murine protein. In particular embodiments, the cognate ligands BAFF and/or APRIL are human. In some embodiments, a variant TACI polypeptide with increased or greater binding affinity to APRIL and/or BAFF will have an increase in binding affinity relative to the reference (e.g., unmodified or wild-type) TACI polypeptide control of at least about 5%, such as at least about 10%, 15%, 20%, 25%, 35%, or 50%. In some embodiments, the increase in binding affinity relative to the reference (e.g., unmodified or wild-type) TACI polypeptide is more than about 1.2-fold, about 1.5-fold, about 2-fold, about 3-fold, about 4-fold, about 5-fold, about 6-fold, about 7-fold, about 8-fold, about 9-fold, about 10-fold, about 20-fold, about 30-fold, about 40-fold or about 50-fold. In any of the examples, the reference (e.g., unmodified or wild-type) TACI polypeptide has the same sequence as the variant TACI polypeptide except that it does not contain the one or more amino acid modifications (e.g., substitutions).

In some embodiments, the equilibrium dissociation constant (K_{d}) of any of the foregoing embodiments to BAFF can be less than 1×10⁻⁵ M, 1×10⁻⁶ M, 1×10⁻⁷ M, 1×10⁻⁸ M, 1×10⁻⁹ M, 1×10⁻¹⁰ M or 1×10⁻¹¹ M, or 1×10⁻¹² M. In some embodiments, the K_{d} of any of the foregoing embodiments to BAFF is less than at or about 1×10⁻⁹ M, 1×10⁻¹⁰ M or 1×10⁻¹¹ M, or 1×10⁻¹² M. In some embodiments, the K_{d} of any of the foregoing embodiments to BAFF is between 1×10⁻⁹ M and at or about 1×10⁻¹ M. In some embodiments, the K_{d} of any of the foregoing embodiments to BAFF is at or about 1×10⁻⁹ M, at or about 2×10⁻⁹ M, at or about 4×10⁻⁹ M, at or about 6×10⁻⁹ M, at or about 8×10⁻⁹ M, at or about 1×10⁻¹⁰ M, at or about 2×10⁻¹⁰ M, at or about 4×10⁻¹⁰ M, at or about 6×10⁻¹⁰ M, at or about 8×10⁻¹⁰ M, at or about 1×10⁻¹¹ M, at or about 2×10⁻¹¹ M, at or about 4×10⁻¹¹ M, at or about 6×10⁻¹¹ M, at or about 8×10⁻¹¹ M, or at or about 1×10⁻¹² M, or any value between any of the foregoing. In some embodiments, a provided embodiment includes a variant TACI polypeptide as described above and the K_{d} to BAFF is decreased (higher binding affinity) by greater than or greater than about 1.5-fold, such as greater than or about 2-fold, 3-fold, 4-fold, 5-fold, 6-fold, 7-fold, 8-fold, 9-fold, 10-fold or more.

In some embodiments, the equilibrium dissociation constant (K_{d}) of any of the foregoing embodiments to APRIL can be less than 1×10⁻⁵ M, 1×10⁻⁶ M, 1×10⁻⁷ M, 1×10⁻⁸ M, 1×10⁻⁹ M, 1×10⁻¹⁰ M or 1×10⁻¹¹ M, or 1×10⁻¹² M. In some embodiments, the K_{d} of any of the foregoing embodiments to APRIL is less than at or about 1×10⁻⁹ M, 1×10⁻¹⁰ M or 1×10⁻¹¹ M, or 1×10⁻¹² M. In some embodiments, the K_{d} of any of the foregoing embodiments to APRIL is between 1×10⁻⁹ M and at or about 1×10⁻¹² M. In some embodiments, the K_{d} of any of the foregoing embodiments to APRIL is at or about 1×10⁻⁹ M, at or about 2×10⁻⁹ M, at or about 4×10⁻⁹ M, at or about 6×10⁻⁹ M, at or about 8×10⁻⁹ M, at or about 1×10⁻¹⁰ M, at or about 2×10⁻¹⁰ M, at or about 4×10⁻¹⁰ M, at or about 6×10⁻¹⁰ M, at or about 8×10⁻¹⁰ M, at or about 1×10⁻¹¹ M, at or about 2×10⁻¹¹ M, at or about 4×10⁻¹¹ M, at or about 6×10⁻¹¹ M, at or about 8×10⁻¹¹ M, or at or about 1×10⁻¹² M, or any value between any of the foregoing. In some embodiments, a provided embodiment includes a variant TACI polypeptide as described above and the K_{d} to APRIL is decreased (higher binding affinity) by greater than or greater than about 1.5-fold, such as greater than or about 2-fold, 3-fold, 4-fold, 5-fold, 6-fold, 7-fold, 8-fold, 9-fold, 10-fold or more.

The reference (e.g., unmodified or wild-type) TACI sequence does not necessarily have to be used as a starting composition to generate variant TACI polypeptides described herein. Therefore, use of the term “modification”, such as “substitution” does not imply that the present embodiments are limited to a particular method of making variant TACI polypeptides or immunomodulatory proteins containing the same. Variant TACI polypeptides can be made, for example, by de novo peptide synthesis and thus does not necessarily require a modification, such as a “substitution”, in the sense of altering a codon to encode for the modification, e.g. substitution. This principle also extends to the terms “addition” and “deletion” of an amino acid residue which likewise do not imply a particular method of making. The means by which the variant TACI polypeptides are designed or created is not limited to any particular method. In some embodiments, however, a reference (e.g., unmodified or wild-type) TACI encoding nucleic acid is mutagenized from reference (e.g., unmodified or wild-type) TACI genetic material and screened for desired specific binding affinity or other functional activity. In some embodiments, a variant TACI polypeptide is synthesized de novo utilizing protein or nucleic acid sequences available at any number of publicly available databases and then subsequently screened. The National Center for Biotechnology Information provides such information, and its website is publicly accessible via the internet as is the UniProtKB database as discussed previously.

Unless stated otherwise, as indicated throughout the present disclosure, the amino acid modification(s) in a variant TACI polypeptide are designated by amino acid position number corresponding to the numbering of positions of the reference ECD sequence set forth in SEQ ID NO:122. It is within the level of a skilled artisan to identify the corresponding position of a modification, e.g. amino acid substitution, in a TACI polypeptide, including portion thereof containing TD (e.g. CRD1 and/or CRD2) thereof, such as by alignment of a reference sequence (e.g. SEQ ID NO:1 or 13) with SEQ ID NO:122. An alignment identifying corresponding residues is exemplified in FIG. 9. In the listing of modifications throughout this disclosure, the amino acid position is indicated in the middle, with the corresponding reference (e.g. unmodified or wild-type) amino acid listed before the number and the identified variant amino acid substitution listed after the number. If the modification is a deletion of the position a “del” is indicated and if the modification is an insertion at the position an “ins” is indicated. In some cases, an insertion is listed with the amino acid position indicated in the middle, with the corresponding reference amino acid listed before and after the number and the identified variant amino acid insertion listed after the unmodified (e.g. wild-type) amino acid.

In some embodiments, the variant TACI polypeptide has one or more amino acid modification, e.g. substitution in a reference (e.g., unmodified or wild-type) TACI sequence, such as any as described. The one or more amino acid modification, e.g. substitution, can be in the ectodomain (extracellular domain) of the reference (e.g., unmodified or wild-type) TACI sequence. In some embodiments, the one or more amino acid modification, e.g. substitution is in the CRD1 domain or specific binding fragment thereof. In some embodiments, the one or more amino acid modification, e.g. substitution is in the CRD2 domain or specific binding fragment thereof. In some embodiments of the variant TACI polypeptide, some of the one or more amino acid modification, e.g. substitution is in the CRD1 domain or a specific binding fragment thereof, and some of the one or more amino acid modification, e.g. substitution are in the CRD2 domain or a specific binding fragment thereof.

In some embodiments, the variant TACI polypeptide has up to 1, 2, 3, 4, 5, 6, 7, 8, 9, 10, 11, 12, 13, 14, 15, 16, 17, 18, 19, or 20 amino acid modification(s), e.g. substitution, in the reference TACI sequence. The modification, e.g. substitution can be in the CRD1 domain or the CRD2 domain. In some embodiments, the variant TACI polypeptide has up to 1, 2, 3, 4, 5, 6, 7, 8, 9, 10, 11, 12, 13, 14, 15, 16, 17, 18, 19, or 20 amino acid substitutions in the CRD1 domain or specific binding fragment thereof of the reference TACI sequence. In some embodiments, the variant TACI polypeptide has up to 1, 2, 3, 4, 5, 6, 7, 8, 9, 10, 11, 12, 13, 14, 15, 16, 17, 18, 19, or 20 amino acid substitutions in the CRD2 domain or specific binding fragment thereof of the reference TACI sequence.

In some embodiments, the variant TACI polypeptide containing the one or more amino acid modifications (e.g. amino acid substations) as described has at least about 85%, 86%, 86%, 88%, 89%, 90%, 91%, 92%, 93%, 94%, 95%, 96%, 97%, 98%, or 99% sequence identity with the reference (e.g., unmodified or wild-type) TACI polypeptide set forth in SEQ ID NO:122 or specific binding fragment thereof containing the CRD1 and/or CRD2 domain. In some embodiments, the specific binding fragment contains the CRD1 domain, e.g. the specific binding fragment contains the sequence set forth as amino acids 34-66 of SEQ ID NO:122. In some cases, the CRD1 domain is the only full CRD domain in the specific binding fragment. In some embodiments, the specific binding fragment is or contains the CRD2 domain, e.g. the specific binding fragment contains the sequence set forth as amino acids 71-104 of SEQ ID NO:122. In some cases, the CRD2 domain is the only full CRD domain in the specific binding fragment. In some embodiments, the specific binding fragment is or contains the CRD1 domain and the CRD2 domain, e.g. the specific binding fragment contains amino acids 34-104 of SEQ ID NO:122. In some embodiments, the specific binding fragment contains a contiguous portion of the stalk domain, e.g. the specific binding fragment contains a contiguous portion of amino acids 105-165 of SEQ ID NO:122. In embodiments of any embodiments, the specific binding fragment of SEQ ID NO:122 is less than the full-length ECD set forth in SEQ ID NO:122. In some embodiments, the specific binding fragment is set forth in SEQ ID NO: 1. In some embodiments, the specific binding fragment is set forth in SEQ ID NO:13. In some embodiments, the specific binding fragment is set forth in SEQ ID NO: 130. In some embodiments, the specific binding fragment is set forth in SEQ ID NO:131.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the reference (e.g., unmodified or wild-type) TACI polypeptide or specific binding fragment thereof, such as with the amino acid sequence of SEQ ID NO: 1, 13 or 122.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 122.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 1.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 13.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 130.

In some embodiments, the variant TACI polypeptide containing the one or more amino acid modifications (e.g. amino acid substitutions) as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 131.

In some embodiments, the variant TACI polypeptide has one or more amino acid modification, e.g. substitution in a reference TACI polypeptide or specific binding fragment there of corresponding to position(s) 40, 59, 60, 61, 74, 75, 76, 77, 78, 79, 82, 83, 84, 85, 86, 87, 88, 92, 95, 97, 98, 99, 101, 102 and 103 with reference to numbering of SEQ ID NO:122. In some embodiments, the variant TACI polypeptide has one or more amino acid modification, e.g. substitution selected from W40R, Q59R, R60G, T61P, E74V, Q75E, Q75R, G76S, K77E, F78Y, Y79F, L82H, L82P, L83S, R84G, R84L, R84Q, D85E, D85V, C86Y, I87L, I87M, S88N, 192V, Q95R, P97S, K98T, Q99E, A101D, Y102D, F103S, F103V, F103Y, or a conservative amino acid substitution thereof. In some embodiments, the reference TACI polypeptide includes the CRD1 domain or CRD2 domain, for example the reference TACI polypeptide is set forth in SEQ ID NO: 1 or SEQ ID NO:122.

In some embodiments, the amino acid substitutions are in the CRD2 domain only. In some embodiments, the variant TACI polypeptide has one or more amino acid modification, e.g. substitution in a reference TACI polypeptide or specific binding fragment there of corresponding to position(s) 74, 75, 76, 77, 78, 79, 82, 83, 84, 85, 86, 87, 88, 92, 95, 97, 98, 99, 101, 102 and 103 with reference to numbering of SEQ ID NO:122. In some embodiments, the variant TACI polypeptide has one or more amino acid modification, e.g. substitution selected from E74V, Q75E, Q75R, G76S, K77E, F78Y, Y79F, L82H, L82P, L83S, R84G, R84L, R84Q, D85E, D85V, C86Y, I87L, I87M, S88N, I92V, Q95R, P97S, K98T, Q99E, A101D, Y102D, F103S, F103V, F103Y, or a conservative amino acid substitution thereof. In some embodiments, among the CRD domains, the reference TACI polypeptide includes only the CRD2 domain but lacks the CRD1 domain, for example the reference TACI polypeptide is set forth in SEQ ID NO: 13. Accordingly, in some embodiments, the variant TACI polypeptide includes a portion of the ECD sequence of a TACI polypeptide that includes the CRD2 domain but lacks the CRD1 domain.

A conservative amino acid modification, e.g. substitution is any amino acid that falls in the same class of amino acids as the substituted amino acids, other than the reference (e.g., unmodified) or wild-type amino acid. The classes of amino acids are aliphatic (glycine, alanine, valine, leucine, and isoleucine), hydroxyl or sulfur-containing (serine, cysteine, threonine, and methionine), cyclic (proline), aromatic (phenylalanine, tyrosine, tryptophan), basic (histidine, lysine, and arginine), and acidic/amide (aspartate, glutamate, asparagine, and glutamine).

In some embodiments, the variant TACI polypeptide includes at least one amino acid substitution at position 75 with reference to numbering of SEQ ID NO:122. In some embodiments, the amino acid substitution at position 75 confers increased binding to BAFF or APRIL compared to the reference (e.g. wildtype or unmodified) TACI polypeptide not containing the amino acid substitution. In some embodiments, the substituted amino acid is an acidic amino acid or amide, such as to a different acidic amino acid or amide compared to the reference (e.g. wildtype or unmodified) TACI polypeptide. In some embodiments, the substituted amino acid at position 75 is a glutamic acid (Glu, E). In some embodiments, the substituted amino acid at position 75 is an aspartic acid (Asp, D). In some embodiments, the substituted amino acid at position 75 is an asparagine (Asn, N). In some embodiments, the substituted amino acid at position 75 is a glutamine (Gln, Q).

In some embodiments, the variant TACI polypeptide includes at least one amino acid substitution at position 77 with reference to numbering of SEQ ID NO:122. In some embodiments, the amino acid substitution at position 77 confers increased binding to BAFF or APRIL compared to the reference (e.g. wildtype or unmodified) TACI polypeptide not containing the amino acid substitution. In some embodiments, the substituted amino acid at position 77 is an acidic amino acid or amide. In some embodiments, the substituted amino acid at position 77 is a glutamic acid (Glu, E). In some embodiments, the substituted amino acid at position 77 is an aspartic acid (Asp, D). In some embodiments, the substituted amino acid at position 77 is an asparagine (Asn, N). In some embodiments, the substituted amino acid at position 77 is a glutamine (Gln, Q).

In some embodiments, the variant TACI polypeptide includes at least one amino acid substitution at position 78 with reference to numbering of SEQ ID NO:122. In some embodiments, the amino acid substitution at position 78 confers increased binding to BAFF or APRIL compared to the reference (e.g. wildtype or unmodified) TACI polypeptide not containing the amino acid substitution. In some embodiments, the substituted amino acid at position 78 is an aromatic amino acid, such as to a different aromatic amino acid compared to the reference (e.g. wildtype or unmodified) TACI polypeptide. In some embodiments, the substituted amino acid at position 78 is a phenylalanine (Phe, F). In some embodiments, the substituted amino acid at position 78 is a tyrosine (Tyr, Y). In some embodiments, the substituted amino acid at position 78 is a tryptophan (Trp, W).

In some embodiments, the variant TACI polypeptide includes at least one amino acid substitution at position 84 with reference to numbering of SEQ ID NO:122. In some embodiments, the amino acid substitution at position 84 confers increased binding to BAFF or APRIL compared to the reference (e.g. wildtype or unmodified) TACI polypeptide not containing the amino acid substitution. In some embodiments, the substituted amino acid at position 84 is an acidic amino acid or amide. In some embodiments, the substituted amino acid at position 84 is a glutamic acid (Glu, E). In some embodiments, the substituted amino acid at position 84 is an aspartic acid (Asp, D). In some embodiments, the substituted amino acid at position 84 is an asparagine (Asn, N). In some embodiments, the substituted amino acid at position 84 is a glutamine (Gln, Q).

In some embodiments, the variant TACI polypeptide includes at least one amino acid substitution at position 101 with reference to numbering of SEQ ID NO:122. In some embodiments, the amino acid substitution at position 101 confers increased binding to BAFF or APRIL compared to the reference (e.g. wildtype or unmodified) TACI polypeptide not containing the amino acid substitution. In some embodiments, the substituted amino acid at position 101 is an acidic amino acid or amide. In some embodiments, the substituted amino acid at position 101 is a glutamic acid (Glu, E). In some embodiments, the substituted amino acid at position 101 is an aspartic acid (Asp, D). In some embodiments, the substituted amino acid at position 101 is an asparagine (Asn, N). In some embodiments, the substituted amino acid at position 101 is a glutamine (Gln, Q).

In some embodiments, the variant TACI polypeptide includes at least one amino acid substitution at position 102 with reference to numbering of SEQ ID NO:122. In some embodiments, the amino acid substitution at position 102 confers increased binding to BAFF or APRIL compared to the reference (e.g. wildtype or unmodified) TACI polypeptide not containing the amino acid substitution. In some embodiments, the substituted amino acid at position 102 is an acidic amino acid or amide. In some embodiments, the substituted amino acid at position 102 is a glutamic acid (Glu, E). In some embodiments, the substituted amino acid at position 102 is an aspartic acid (Asp, D). In some embodiments, the substituted amino acid at position 102 is an asparagine (Asn, N). In some embodiments, the substituted amino acid at position 102 is a glutamine (Gln, Q).

In some embodiments, the variant TACI polypeptide includes at least one amino acid substitution E74V. In some embodiments, the variant TACI polypeptide includes at least one amino acid substitution Q75E. In some embodiments, the variant TACI polypeptide includes at least one amino acid substitution K77E. In some embodiments, the variant TACI polypeptide includes at least one amino acid substitution F78Y. In some embodiments, the variant TACI polypeptide includes at least one amino acid substitution Y79F. In some embodiments, the variant TACI polypeptide includes at least one amino acid substitution L82H. In some embodiments, the variant TACI polypeptide includes at least one amino acid substitution L82P. In some embodiments, the variant TACI polypeptide includes at least one amino acid substitution R84G. In some embodiments, the variant TACI polypeptide includes at least one amino acid substitution R84L. In some embodiments, the variant TACI polypeptide includes at least one amino acid substitution R84Q. In some embodiments, the variant TACI polypeptide includes at least one amino acid substitution D85V. In some embodiments, the variant TACI polypeptide includes at least one amino acid substitution C86Y. In some embodiments, the variant TACI polypeptide includes at least one amino acid substitution A101D. In some embodiments, the variant TACI polypeptide includes at least one amino acid substitution Y102D. In some embodiments, the variant TACI polypeptide contains two or more amino acid substitutions of any two or more of the foregoing. In some embodiments, the variant TACI polypeptide includes one or more amino acid substitution that is a conservative amino acid substitution of any of the foregoing. In provided embodiments, the variant TACI polypeptide includes the at least one amino acid substitution in any reference TACI polypeptide sequence as described. In some embodiments, the at least one amino acid substitution is in the reference TACI sequence set forth in SEQ ID NO: 1. In some embodiments, the at least one amino acid substitution is in the reference TACI sequence set forth in SEQ ID NO: 13. In some embodiments, the at least one amino acid substitution is in the reference TACI sequence set forth in SEQ ID NO: 130. In some embodiments, the at least one amino acid substitution is in the reference TACI sequence set forth in SEQ ID NO: 131.

In some embodiments, the variant TACI polypeptide includes the amino acid substitution E74V. In some embodiments, the variant TACI polypeptide includes the amino acid substitution Q75E. In some embodiments, the variant TACI polypeptide includes the amino acid substitution K77E. In some embodiments, the variant TACI polypeptide includes the amino acid substitution F78Y. In some embodiments, the variant TACI polypeptide includes the amino acid substitution Y79F. In some embodiments, the variant TACI polypeptide includes the amino acid substitution L82H. In some embodiments, the variant TACI polypeptide includes the amino acid substitution L82P. In some embodiments, the variant TACI polypeptide includes the amino acid substitution R84G. In some embodiments, the variant TACI polypeptide includes the amino acid substitution R84L. In some embodiments, the variant TACI polypeptide includes the amino acid substitution R84Q. In some embodiments, the variant TACI polypeptide includes the amino acid substitution D85V. In some embodiments, the variant TACI polypeptide includes the amino acid substitution C86Y. In some embodiments, the variant TACI polypeptide includes the amino acid substitution A102D. In some embodiments, the variant TACI polypeptide includes the amino acid substitution Y102D. In some embodiments, the variant TACI polypeptide contains two or more amino acid substitutions of any two or more of the foregoing. In some embodiments, the variant TACI polypeptide includes one or more of amino acid substitution that is a conservative amino acid substitution of any of the foregoing. In provided embodiments, the variant TACI polypeptide includes the amino acid substitution in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments, the amino acid substitutions are D85E/K98T. In some embodiments, the amino acid substitutions are I87L/K98T. In some embodiments, the amino acid substitutions are R60G/Q75E/L82P. In some embodiments, the amino acid substitutions are R60G/C86Y. In some embodiments, the amino acid substitutions are W40R/L82P/F103Y. In some embodiments, the amino acid substitutions are W40R/Q59R/T61P/K98T. In some embodiments, the amino acid substitutions are L82P/I87L. In some embodiments, the amino acid substitutions are G76S/P97S. In some embodiments, the amino acid substitutions are K77E/R84L/F103Y. In some embodiments, the amino acid substitutions are Y79F/Q99E. In some embodiments, the amino acid substitutions are L83S/F103S. In some embodiments, the amino acid substitutions are K77E/R84Q. In some embodiments, the amino acid substitutions are K77E/A101D. In some embodiments, the amino acid substitutions are K77E/F78Y/Y102D. In some embodiments, the amino acid substitutions are Q75E/R84Q. In some embodiments, the amino acid substitutions are Q75R/R84G/I92V. In some embodiments, the amino acid substitutions are K77E/A101D/Y102D. In some embodiments, the amino acid substitutions are R84Q/S88N/A101D. In some embodiments, the amino acid substitutions are R84Q/F103V. In some embodiments, the amino acid substitutions are K77E/Q95R/A101D. In some embodiments, the amino acid substitutions are I87M/A101D. In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In embodiments of any embodiments, the variant TACI polypeptide includes one or more amino acid substitutions from Q75E, K77E, F78Y, R84G, R84Q, A101D or Y102D, or any combination thereof. In some embodiments, the variant TACI polypeptide includes any 1, 2, 3, 4, 5 or 6 of the above amino acid substitutions. In some embodiments, the variant TACI polypeptide contains one of the above amino acid substitutions. In some embodiments, the variant TACI polypeptide contains two of the above amino acid substitutions. In some embodiments, the variant TACI polypeptide contains three of the above amino acid substitutions. In some embodiments, the variant TACI polypeptide contains four of the above amino acid substitutions. In some embodiments, the variant TACI polypeptide contains five of the above amino acid substitutions. In some embodiments, the variant TACI polypeptide contains six of the above amino acid substitutions.

In embodiments of any embodiments, the one or more amino acid substitutions comprise Q75E/R84Q. In embodiments of any embodiments, the one or more amino acid substitutions comprise Q75E/K77E. In embodiments of any embodiments, the one or more amino acid substitutions comprise Q75E/F78Y. In embodiments of any embodiments, the one or more amino acid substitutions comprise Q75E/A101D. In embodiments of any embodiments, the one or more amino acid substitutions comprise Q75E/Y102D. In embodiments of any embodiments, the one or more amino acid substitutions comprise F77E/F78Y. In embodiments of any embodiments, the one or more amino acid substitutions comprise K77E/R84Q. In embodiments of any embodiments, the one or more amino acid substitutions comprise K77E/A101D. In embodiments of any embodiments, the one more amino acid substitutions comprise K77E/Y102D. In embodiments of any embodiments, the one or more amino acid substitutions comprise F78Y/R84Q. In embodiments of any embodiments, the one or more amino acid substitutions comprise F78Y/A101D. In embodiments of any embodiments, the one or more amino acid substitutions comprise F78Y/Y102D. In embodiments of any embodiments, the one or more amino acid substitutions comprise R84Q/A101D. In embodiments of any embodiments, the one or more amino acid substitutions comprise R84Q/Y102D. In embodiments of any embodiments, the one or more amino acid substitutions comprise A101D/Y102D. In provided embodiments, the variant TACI polypeptide includes the amino acid substitutions in any reference TACI polypeptide sequence as described, such as in the sequence set forth in SEQ ID NO:1, SEQ ID NO:13, SEQ ID NO:130 or SEQ ID NO: 131.

In some embodiments, the variant TACI polypeptides include the amino acid substitution(s) R84G, A101D, K77E/R84Q, K77E/A101D, K77E/F78Y, K77E/F78Y/Y102D, Q75E/R84Q, K77E/A101D/Y102D, R84Q, K77E, A101D, Q75E, K77E/F78Y/R84Q, F78Y, F78Y/R84Q, F78Y/A101D, F78Y/Y102D, or K77E/Y102D. In provided embodiments, the variant TACI polypeptide includes the amino acid substitutions in any reference TACI polypeptide sequence as described, such as in the sequence set forth in SEQ ID NO:1, SEQ ID NO:13, SEQ ID NO:130 or SEQ ID NO: 131.

In some embodiments, the variant TACI polypeptide includes the amino acid substitutions K77E and F78Y (K77E/F78Y). In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments, the variant TACI polypeptide includes the amino acid substitutions K77E and Y102D (K77E/Y102D). In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments, the variant TACI polypeptide contains the amino acid substitutions F78Y and Y102D (F78Y/Y012D). In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments the variant TACI polypeptide contains the amino acid substitutions K77E, F78Y and Y102D (K77E/F78Y/Y102D). In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments, the variant TACI polypeptide contains the amino acid substitutions Q75E/R84Q. In provided embodiments, the variant TACI polypeptide includes the amino acid substitutions in any reference TACI polypeptide sequence as described. In some embodiments, the amino acid substitution is in the reference TACI sequence set forth in SEQ ID NO: 1. In some embodiments, the amino acid substitution is in the reference TACI sequence set forth in SEQ ID NO: 13. In some embodiments, the amino acid substitution is in the reference TACI sequence set forth in SEQ ID NO: 130. In some embodiments, the amino acid substitution is in the reference TACI sequence set forth in SEQ ID NO: 131.

In some embodiments, the variant TACI polypeptide comprises any of the mutations listed in Table 1. Table 1 also provides exemplary sequences by reference to SEQ ID NO of the reference (e.g., unmodified) TACI polypeptide, and exemplary variant TACI polypeptides. As indicated, the exact locus or residues corresponding to a given domain can vary, such as depending on the methods used to identify or classify the domain. Also, in some cases, adjacent N- and/or C-terminal amino acids of a given domain (e.g. CRD) also can be included in a sequence of a variant TACI polypeptide, such as to ensure proper folding of the domain when expressed. Thus, it is understood that the exemplification of the SEQ ID NOs in Table 1 is not to be construed as limiting. For example, the particular domain, such as the ECD domain or a portion thereof containing the CRD1/CRD2 or CRD2 only, of a variant TACI polypeptide can be several amino acids longer or shorter, such as 1-10, e.g., 1, 2, 3, 4, 5, 6 or 7 amino acids longer or shorter, than the sequence of amino acids set forth in the respective SEQ ID NO.

In some embodiments, the variant TACI polypeptide comprises any of the mutations (amino acid substitutions) listed in Table 1. In some examples, the mutations (amino acid substitutions) are made in a reference TACI containing the sequence of amino acids set forth in SEQ ID NO: 122. In some examples, the mutations (amino acid substitutions) are made a reference TACI that contains the CRD1 and CRD2 domain of TACI, for example as set forth in SEQ ID NO: 1. In some examples, the mutations (amino acid substitutions) are made in a reference TACI that is further truncated by deletion of N-terminal and C-terminal amino acid residues to retain the CRD2, for example as set forth in SEQ ID NO: 13.

The use of the term “modification”, such as “substitution” or “mutation,” does not imply that the present embodiments are limited to a particular method of making the immunomodulatory proteins. A variant TACI polypeptide can be made, for example, by de novo peptide synthesis and thus does not necessarily require a modification, such as a “substitution” in the sense of altering a codon to encode for the modification, e.g. substitution. This principle also extends to the terms “addition” and “deletion” of an amino acid residue which likewise do not imply a particular method of making. The means by which the vTDs are designed or created is not limited to any particular method. In some embodiments, however, a wild-type or unmodified TD encoding nucleic acid is mutagenized from wild-type or unmodified TD genetic material and screened for desired specific binding activity, e.g. binding affinity, and/or alteration of NF-κB modulation or other functional activity. In some embodiments, a vTD is synthesized de novo utilizing protein or nucleic acid sequences available at any number of publicly available databases and then subsequently screened. The National Center for Biotechnology Information provides such information and its website is publicly accessible via the internet as is the UniProtKB database.

In some embodiments, the variant TACI polypeptide comprises an extracellular domain (ECD) sequence containing a CRD1 and CRD2, such as a variant TACI polypeptide set forth in any one of SEQ ID NOS: 2-12, 21, 22, 101-120. In some embodiments, the variant TACI polypeptide comprises a polypeptide sequence that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 2-12, 21, 22, 101-120, and retains the amino acid modification(s), e.g. substitution(s) therein not present in the reference (e.g., unmodified or wild-type) TAC. In some embodiments, the variant TACI polypeptide comprises a specific binding fragment of any one of SEQ ID NOS: 2-12, 21, 22, 101-120, in which the specific binding fragment binds BAFF, APRIL or a BAFF/APRIL heterotrimer, and contains a contiguous sequence therein that contains the amino acid modification(s), e.g. substitution (s) therein not present in the reference (e.g., unmodified or wild-type) TACI.

In some embodiments, the variant TACI polypeptide consists or consists essentially of a variant TACI extracellular domain (ECD) sequence set forth in any one of SEQ ID NOS: 2-12, 21, 22, 101-120. In some embodiments, the variant TACI polypeptide consists or consists essentially of a polypeptide sequence that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 2-12, 21, 22, 101-120, and retains the amino acid modification(s), e.g. substitution(s) therein not present in the reference (e.g., unmodified or wild-type) TAC. In some embodiments, the variant TACI polypeptide consists or consists essentially of a specific binding fragment of any one of SEQ ID NOS: 2-12, 21, 22, 101-120, in which the specific binding fragment binds BAFF, APRIL or an APRIL/BAFF heterotrimer and contains a contiguous sequence therein that contains the amino acid modification(s), e.g. substitution (s) therein not present in the reference (e.g., unmodified or wild-type) TACI.

In some embodiments, the variant TACI polypeptide comprises an extracellular domain (ECD) sequence containing a CRD2 but lacking the CRD1 of a reference TACI polypeptide, such as a variant TACI polypeptide set forth in any one of SEQ ID NOS: 14-20, 23-35, 92-100, 177-192. In some embodiments, the variant TACI polypeptide comprises a polypeptide sequence that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 14-20, 23-35, 92-100, 177-192, and retains the amino acid modification(s), e.g. substitution(s) therein not present in the reference (e.g., unmodified or wild-type) TAC. In some embodiments, the variant TACI polypeptide comprises a specific binding fragment of any one of SEQ ID NOS: 14-20, 23-35, 92-100, 177-192 in which the specific binding fragment binds BAFF, APRIL or a BAFF/APRIL heterotrimer, and contains a contiguous sequence therein that contains the amino acid modification(s), e.g. substitution (s) therein not present in the reference (e.g., unmodified or wild-type) TACI.

In some embodiments, the variant TACI polypeptide consists or consists essentially of the sequence set forth in any one of SEQ ID NOS: 14-20, 23-35, 92-100, 177-192. In some embodiments, the variant TACI polypeptide consists or consists essentially of a polypeptide sequence that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 14-20, 23-35, 92-100, 177-192, and retains the amino acid modification(s), e.g. substitution(s) therein not present in the reference (e.g., unmodified or wild-type) TAC. In some embodiments, the variant TACI polypeptide consists or consists essentially of a specific binding fragment of any one of SEQ ID NOS: 14-20, 23-35, 92-100, 177-192, in which the specific binding fragment binds BAFF, APRIL or a BAFF/APRIL heterotrimer, and contains a contiguous sequence therein that contains the amino acid modification(s), e.g. substitution (s) therein not present in the reference (e.g., unmodified or wild-type) TACI.

In some embodiments, the variant TACI polypeptide comprises the sequence set forth in SEQ ID NO:20. In some embodiments, the variant TACI polypeptide consists essentially of the sequence set forth in SEQ ID NO:20. In some embodiments, the variant TACI polypeptide consists of the sequence set forth in SEQ ID NO:20.

In some embodiments, the variant TACI polypeptide comprises the sequence set forth in SEQ ID NO:26. In some embodiments, the variant TACI polypeptide consists essentially of the sequence set forth in SEQ ID NO:26. In some embodiments, the variant TACI polypeptide consists of the sequence set forth in SEQ ID NO:26.

In some embodiments, the variant TACI polypeptide comprises the sequence set forth in SEQ ID NO:27. In some embodiments, the variant TACI polypeptide consists essentially of the sequence set forth in SEQ ID NO:27. In some embodiments, the variant TACI polypeptide consists of the sequence set forth in SEQ ID NO:27.

In some embodiments, the variant TACI polypeptide comprises the sequence set forth in SEQ ID NO:107. In some embodiments, the variant TACI polypeptide consists essentially of the sequence set forth in SEQ ID NO:107. In some embodiments, the variant TACI polypeptide consists of the sequence set forth in SEQ ID NO:107.

In some embodiments, the variant TACI polypeptide is encoded by a sequence of nucleotides set forth in any of SEQ ID NOS: 37-47, 56 or 57. In some embodiments, the variant TACI polypeptide is encoded by a sequence of nucleotides that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 37-47, 56 or 57, and retains the amino acid modification(s), e.g. substitution(s) therein not present in the reference (e.g., unmodified or wild-type) TAC. Also provided herein is a nucleic acid containing the sequence set forth in any of SEQ ID NOS: 37-47, 56 or 57 or a sequence that exhibits at least 90% identity, at least 91% identity, at least 92% identity, at least 93% identity, at least 94% identity, at least 95% identity, such as at least 96% identity, 97% identity, 98% identity, or 99% identity to any one of SEQ ID NOS: 37-47, 56 or 57.

In some embodiments, the variant TACI polypeptide is encoded by a sequence of nucleotides set forth in any of SEQ ID NOS: 49-55 or 58-70. In some embodiments, the variant TACI polypeptide is encoded by a sequence of nucleotides that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 49-55 or 58-70, and retains the amino acid modification(s), e.g. substitution(s) therein not present in the reference (e.g., unmodified or wild-type) TAC. Also provided herein is a nucleic acid containing the sequence set forth in any of SEQ ID NOS: 49-55 or 58-70 or a sequence that exhibits at least 90% identity, at least 91% identity, at least 92% identity, at least 93% identity, at least 94% identity, at least 95% identity, such as at least 96% identity, 97% identity, 98% identity, or 99% identity to any one of SEQ ID NOS: 549-55 or 58-70.

TABLE 1
Exemplary variant TACI

| | | ECD | | ECD | |
|---|---|---|---|---|---|
| | | (CRD1/CRD2) | | (CRD2) | |
| | | AA | NT | AA | NT |
| | | SEQ | SEQ | SEQ | SEQ |
| Name | Mutation(s) | ID NO | ID NO | ID NO | ID NO |
| 1 (WT) TACI CRD1/CRD2 | Wild-type | 1 | 36 | 13 | 48 |
| 13 (WT) TACI CRD2 | | | | | |
| 2 TACI CRD1/CRD2 | L82P | 2 | 37 | 92 | |
| 92 TACI CRD2 | | | | | |
| 3 TACI CRD1/CRD2 | D85E, K98T | 3 | 38 | 93 | |
| 93 TACI CRD2 | | | | | |
| 4 TACI CRD1/CRD2 | I87L, K98T | 4 | 39 | 94 | |
| 94 TACI CRD2 | | | | | |
| 5 TACI CRD1/CRD2 | R60G, Q75E, L82P | 5 | 40 | | |
| 6 TACI CRD1/CRD2 | R60G, C86Y | 6 | 41 | | |
| 7 TACI CRD1/CRD2 | A101D | 7 | 42 | 95 | |
| 95 TACI CRD2 | | | | | |
| 8 TACI CRD1/CRD2 | C86Y | 8 | 43 | 96 | |
| 96 TACI CRD2 | | | | | |
| 9 TACI CRD1/CRD2 | W40R, L82P, F103Y | 9 | 44 | | |
| 10 TACI CRD1/CRD2 | W40R, Q59R, T61P, K98T | 10 | 45 | | |
| 11 TACI CRD1/CRD2 | L82P, I87L | 11 | 46 | 97 | |
| 97 TACI CRD2 | | | | | |
| 12 TACI CRD1/CRD2 | G76S, P97S | 12 | 47 | 98 | |
| 98 TACI CRD2 | | | | | |
| 101 TACI CRD1/CRD2 | D85V | 101 | | 14 | 49 |
| 14 TACI CRD2 | | | | | |
| 102 TACI CRD1/CRD2 | E74V | 102 | | 15 | 50 |
| 15 TACI CRD2 | | | | | |
| 103 TACI CRD1/CRD2 | R84L | 103 | | 16 | 51 |
| 16 TACI CRD2 | | | | | |
| 104 TACI CRD1/CRD2 | K77E, R84L, F103Y | 104 | | 17 | 52 |
| 17 TACI CRD2 | | | | | |
| 105 TACI CRD1/CRD2 | Y79F, Q99E | 105 | | 18 | 53 |
| 18 TACI CRD2 | | | | | |
| 106 TACI CRD1/CRD2 | Y79F | 106 | | 19 | 54 |
| 19TACI CRD2 | | | | | |
| 107 TACI CRD1/CRD2 | R84G | 107 | | 20 | 55 |
| 20 TACI CRD2 | | | | | |
| 21 TACI CRD1/CRD2 | L83S, F103S | 21 | 56 | 99 | |
| 99 TACI CRD2 | | | | | |
| 22 TACI CRD1/CRD2 | L82H | 22 | 57 | 100 | |
| 100 TACI CRD2 | | | | | |
| 108 TACI CRD1/CRD2 | A101D | 108 | | 23 | 58 |
| 23 TACI CRD2 | | | | | |
| 109 TACI CRD1/CRD2 | K77E, R84Q | 109 | | 24 | 59 |
| 24 TACI CRD2 | | | | | |
| 110 TACI CRD1/CRD2 | K77E, A101D | 110 | | 25 | 60 |
| 25 TACI CRD2 | | | | | |
| 111 TACI CRD1/CRD2 | K77E, F78Y, Y102D | 111 | | 26 | 61 |
| 26 TACI CRD2 | | | | | |
| 112 TACI CRD1/CRD2 | Q75E, R84Q | 112 | | 27 | 62 |
| 27 TACI CRD2 | | | | | |
| 113 TACI CRD1/CRD2 | Q75R, R84G, I92V | 113 | | 28 | 63 |
| 28 TACI CRD2 | | | | | |
| 114 TACI CRD1/CRD2 | K77E, A101D, Y102D | 114 | | 29 | 64 |
| 29 TACI CRD2 | | | | | |
| 115 TACI CRD1/CRD2 | R84Q | 115 | | 30 | 65 |
| 30 TACI CRD2 | | | | | |
| 116 TACI CRD1/CRD2 | R84Q, S88N, A101D | 116 | | 31 | 66 |
| 31 TACI CRD2 | | | | | |
| 117 TACI CRD1/CRD2 | K77E | 117 | | 32 | 67 |
| 32 TACI CRD2 | | | | | |
| 118 TACI CRD1/CRD2 | R84Q, F103V | 118 | | 33 | 68 |
| 33 TACI CRD2 | | | | | |
| 119 TACI CRD1/CRD2 | K77E, Q95R, A101D | 119 | | 34 | 69 |
| 34 TACI CRD2 | | | | | |
| 120 TACI CRD1/CRD2 | I87M, A101D | 120 | | 35 | 70 |
| 35 TACI CRD2 | | | | | |
| 177 TACI CRD2 | Q75E | | | 177 | |
| 178 TACI CRD2 | Q75E, K77E | | | 178 | |
| 179 TACI CRD2 | Q75E, F78Y | | | 179 | |
| 180 TACI CRD2 | Q75E, A101D | | | 180 | |
| 181 TACI CRD2 | Q75E, Y102D | | | 181 | |
| 182 TACI CRD2 | K77E, F78Y, R84Q | | | 182 | |
| 183 TACI CRD2 | F78Y | | | 183 | |
| 184 TACI CRD2 | F78Y, R84Q | | | 184 | |
| 185 TACI CRD2 | F78Y, A101D | | | 185 | |
| 186 TACI CRD2 | F78Y, Y102D | | | 186 | |
| 187 TACI CRD2 | R84Q, A101D | | | 187 | |
| 188 TACI CRD2 | R84Q, Y102D | | | 188 | |
| 189 TACI CRD2 | A101D, Y102D | | | 189 | |
| 190 TACI CRD2 | Y102D | | | 190 | |
| 191 TACI CRD2 | K77E, F78Y | | | 191 | |
| 192 TACI CRD2 | K77E, Y102D | | | 192 | |

In some embodiments, also provided herein are TACI ECD fusion sequences in which any of the above TACI ECD sequence is linked or fused to a multimerization domain, such as any described herein.

Interaction of two or more polypeptides of the immunomodulatory proteins can be facilitated by their linkage, either directly or indirectly, to any moiety or other polypeptide that are themselves able to interact to form a stable structure. For example, separate encoded polypeptide chains can be joined by multimerization, whereby multimerization of the polypeptides is mediated by a multimerization domain. Typically, the multimerization domain provides for the formation of a stable protein-protein interaction between a first polypeptide and a second polypeptide.

In some embodiments, the two or more individual polypeptides of the immunomodulatory proteins can be joined by multimerization, such as joined as dimeric, trimeric, tetrameric, or pentameric molecules. In some cases, the individual polypeptides are the same. For example, a trimeric molecule can be formed from three copies of the same individual polypeptide. In other examples, a tetrameric molecule is generated from four copies of the same individual polypeptides. In further examples, a pentameric molecule is generated from five copies of the same individual polypeptides. The multimerization domain may be one that facilities dimerization, trimerization, tetramerization, or pentamerization of the polypeptide chains.

In some embodiments, the immunomodulatory protein forms a multimer, e.g., a dimer. In some embodiments, the dimer is a homodimer in which the two polypeptides of the immunomodulatory protein are the same. In some embodiments, the dimer is a heterodimer in which the two polypeptides of the immunomodulatory protein are different.

In some embodiments, a multimerization domain includes any capable of forming a stable protein-protein interaction. The multimerization domains can interact via an immunoglobulin sequence (e.g. Fc domain; see e.g., International Patent Pub. Nos. WO 93/10151 and WO 2005/063816 US; U.S. Pub. No. 2006/0024298; U.S. Pat. No. 5,457,035); leucine zipper (e.g. from nuclear transforming proteins fos and jun or the proto-oncogene c-myc or from General Control of Nitrogen (GCN4)) (e.g., Busch and Sassone-Corsi (1990) Trends Genetics, 6:36-40; Gentz et al., (1989) Science, 243:1695-1699); a hydrophobic region; a hydrophilic region; or a free thiol which forms an intermolecular disulfide bond between the chimeric molecules of a homo- or heteromultimer. In addition, a multimerization domain can include an amino acid sequence comprising a protuberance complementary to an amino acid sequence comprising a hole, such as is described, for example, in U.S. Pat. No. 5,731,168; International Patent Pub. Nos. WO 98/50431 and WO 2005/063816; Ridgway et al. (1996) Protein Engineering, 9:617-621. Such a multimerization region can be engineered such that steric interactions not only promote stable interaction, but further promote the formation of heterodimers over homodimers from a mixture of chimeric monomers. Generally, protuberances are constructed by replacing small amino acid side chains from the interface of the first polypeptide with larger side chains (e.g., tyrosine or tryptophan). Compensatory cavities of identical or similar size to the protuberances are optionally created on the interface of the second polypeptide by replacing large amino acid side chains with smaller ones (e.g., alanine or threonine). Exemplary multimerization domains are described below.

The TACI polypeptide sequence (e.g. variant TACI polypeptide sequence) can be joined anywhere, but typically via its N- or C-terminus, to the N- or C-terminus of a multimerization domain to form a chimeric polypeptide. The linkage can be direct or indirect via a linker. Also, the chimeric polypeptide can be a fusion protein or can be formed by chemical linkage, such as through covalent or non-covalent interactions. For example, when preparing a chimeric polypeptide containing a multimerization domain, nucleic acid encoding all or part of a TACI polypeptide sequence such as any described TACI ECD, including a variant TACI polypeptide sequence, can be operably linked to nucleic acid encoding the multimerization domain sequence, directly or indirectly or optionally via a linker domain. In some cases, the construct encodes a chimeric protein where the C-terminus of the TACI polypeptide sequence is joined to the N-terminus of the multimerization domain. In some instances, a construct can encode a chimeric protein where the N-terminus of the TACI polypeptide sequence is joined to the N- or C-terminus of the multimerization domain.

A polypeptide multimer contains two chimeric proteins created by linking, directly or indirectly, two of the same or different TACI polypeptide sequences (e.g. two of the same or different variant TACI polypeptide sequences) directly or indirectly to a multimerization domain. In some examples, where the multimerization domain is a polypeptide, a gene fusion encoding the TACI polypeptide sequence (e.g. variant TACI polypeptide sequence) and multimerization domain is inserted into an appropriate expression vector. The resulting chimeric or fusion protein can be expressed in host cells transformed with the recombinant expression vector, and allowed to assemble into multimers, where the multimerization domains interact to form multivalent polypeptides. Chemical linkage of multimerization domains to the TACI polypeptide (e.g. variant TACI polypeptide) can be effected using heterobifunctional linkers.

The resulting chimeric polypeptides, such as fusion proteins, and multimers formed therefrom, can be purified by any suitable method such as, for example, by affinity chromatography over Protein A or Protein G columns. Where two nucleic acid molecules encoding different polypeptides are transformed into cells, formation of homo- and heterodimers will occur. Conditions for expression can be adjusted so that heterodimer formation is favored over homodimer formation.

In some embodiments, the multimerization domain is an Fc region of an immunoglobulin.

In some embodiments, the multimerization domain is an immunoglobulin (e.g. IgG1) Fc region, in which the fusion protein is a TACI-Fc containing (1) a TACI sequence containing or consisting of any of the provided TACI ECD sequences; and (2) an immunoglobulin Fc region. Thus, among provided embodiments are TACI-Fc fusion proteins containing (1) a TACI sequence containing or consisting of any of the above-described TACI ECD polypeptide sequences, such as variant TACI polypeptide; and (2) an immunoglobulin Fc region.

In some embodiments, provided herein is a TACI-Fc fusion sequence that contains (1) a TACI ECD sequence that comprises the sequence set forth in SEQ ID NO:13, and (2) an immunoglobulin Fc region. In some embodiments, provided herein is a TACI-Fc fusion sequence that contains (1) a TACI ECD sequence that consists or consists essentially of the sequence set forth in SEQ ID NO:13, and (2) an immunoglobulin Fc region.

In some embodiments, the TACI-Fc fusion is a variant TACI-Fc fusion containing or consisting of any of the above-described variant TACI polypeptides and an immunoglobulin Fc region.

In some embodiments, provided herein is a variant TACI-Fc fusion sequence that contains (1) a TACI ECD sequence containing a CRD1 and a CRD2, for example a TACI sequence that contains the sequence set forth in any one of SEQ ID NOS: 2-12, 21, 22, 101-120, and (2) an immunoglobulin Fc region. In some embodiments, provided herein is a variant TACI-Fc fusion sequence that contains (1) a TACI ECD sequence containing a CRD1 and a CRD2, for example a TACI sequence that consist or consists essentially of the sequence set forth in any one of SEQ ID NOS: 2-12, 21, 22, 101-120, and (2) an immunoglobulin Fc region.

In some embodiments, provided herein is a variant TACI-Fc fusion sequence that contains (1) a TACI ECD sequence containing the CRD2 but lacking the CRD1 domain, for example a TACI sequence that contains the sequence set forth in any one of SEQ ID NOS: 14-20, 23-35, 92-100, 177-192 and (2) an immunoglobulin Fc region. In some embodiments, provided herein is a variant TACI-Fc fusion sequence that contains (1) a TACI ECD sequence containing the CRD2 domain but lacking the CRD1 domain, for example a TACI sequence that consists or consists essentially of the sequence set forth in any one of SEQ ID NOS: 14-20, 23-35, 92-100, 177-192 and (2) an immunoglobulin Fc region.

In provided embodiments of a TACI-Fc, the immunoglobulin Fc region can be a wild-type Fc of an immunoglobulin, such as an IgG1 Fc. In some cases, the Fc region can be a variant Fc that lacks effector function (also called “effectorless Fc”). Exemplary Fc regions and variants thereof in provided TACI-Fc fusion proteins are described below.

In some embodiments, the Fc is murine or human Fc. In some embodiments, the Fc is a mammalian or human IgG1, IgG2, IgG3, or IgG4 Fc regions.

In some embodiments, the Fc region is or comprises the sequence set forth in any one of SEQ ID NOs: 71, 73, 75, 81, 82, 83, 134, 135, 136, 137, 138, 139, 140, 173, 174, 175, 176, 193, 218, 219, 220, or 221. In some embodiments, the Fc region is or is derived from an IgG1, such as set forth in any one of SEQ ID NOS: 71, 73, 75, 81, 82, 83, 134, 135, 136, 137, 139, 140, 173, 174, 175, 176, 193, 218, 220, or 221. In some embodiments, the Fc region is or is derived from an IgG2, such as any set forth in SEQ ID NO: 138 or 219. In some embodiments, the Fc region is or is derived from an IgG4, such as any set forth in SEQ ID NO: 139, 140 or 220. In some embodiments, an Fc region in Fc fusion proteins provided herein also can include an Fc region that exhibits at least about 85%, 86%, 87%, 88%, 89%, 90%, 91%, 92%, 93%, 94%, 95%, 96%, 97%, 98%, 99% to any of the above Fc regions.

In some embodiments, the Fc is derived from IgG1, such as human IgG1. In some embodiments, the Fc is an IgG1 Fc set forth in SEQ ID NO: 71 having an allotype containing residues Glu (E) and Met (M) at positions 356 and 358 by EU numbering. In some embodiments, the Fc comprises the amino acid sequence set forth in SEQ ID NO: 71 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 71. In other embodiments, the Fc is an IgG1 Fc that contains amino acids of the human G1m1 allotype, such as residues containing Asp (D) and Leu (L) at positions 356 and 358, e.g. as set forth in SEQ ID NO:81. Thus, in some cases, an Fc provided herein can contain amino acid substitutions E356D and M358L to reconstitute residues of allotype G1 ml. In some embodiments, the Fc comprises the amino acid sequence set forth in SEQ ID NO: 81 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 81.

In some embodiments, the Fc region has the amino acid sequence set forth in SEQ ID NO:81.

(SEQ ID NO: 81)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the Fc region comprises the amino acid sequence set forth in SEQ ID NO:81. In some embodiments, the Fc region consists of the amino acid sequence set forth in SEQ ID NO:81.

In some embodiments, the variant Fc comprises the sequence set forth in SEQ ID NO: 173. In some embodiments, the variant Fc comprises the sequence set forth in SEQ ID NO:174. In some embodiments, an Fc region used in a construct provided herein can further lack a C-terminal lysine residue.

In some embodiments, the Fc is derived from IgG2, such as human IgG2. In some embodiments, the Fc comprises the amino acid sequence set forth in SEQ ID NO: 138 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 138. In some embodiments, the Fc region is an IgG2 Fc region that has the sequence set forth in SEQ ID NO: 138. In some embodiments, the Fc region is an IgG2 Fc region that has the sequence set forth in SEQ ID NO: 219.

In some embodiments, the Fc is derived from IgG4, such as human IgG4. In some embodiments, the Fc comprises the amino acid sequence set forth in SEQ ID NO: 139 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 139. In some embodiments, the IgG4 Fc is a stabilized Fc in which the CH3 domain of human IgG4 is substituted with the CH3 domain of human IgG1 and which exhibits inhibited aggregate formation, an antibody in which the CH3 and CH2 domains of human IgG4 are substituted with the CH3 and CH2 domains of human IgG1, respectively, or an antibody in which arginine at position 409 indicated in the EU index proposed by Kabat et al. of human IgG4 is substituted with lysine and which exhibits inhibited aggregate formation (see e.g. U.S. Pat. No. 8,911,726. In some embodiments, the Fc is an IgG4 containing the S228P mutation, which has been shown to prevent recombination between a therapeutic antibody and an endogenous IgG4 by Fab-arm exchange (see e.g. Labrijin et al. (2009) Nat. Biotechnol., 27(8): 767-71.) In some embodiments, the Fc comprises the amino acid sequence set forth in SEQ ID NO: 140 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 140. In some embodiments, the Fc region is an IgG4 Fc region set forth in SEQ ID NO:140. In some embodiments, the Fc region is an IgG4 Fc region set forth in SEQ ID NO:220.

In some embodiments, the Fc region is a variant Fc region in which a wild-type Fc is modified by one or more amino acid substitutions to reduce effector activity or to render the Fc inert for Fc effector function. Exemplary effectorless or inert mutations include those described herein.

In some embodiments, the Fc region contains one more modifications that alter (e.g. reduce) one or more of its normal functions. In general, the Fc region is responsible for effector functions, such as complement-dependent cytotoxicity (CDC) and antibody-dependent cell cytotoxicity (ADCC), in addition to the antigen-binding capacity, which is the main function of immunoglobulins. Additionally, the FcRn sequence present in the Fc region plays the role of regulating the IgG level in serum by increasing the in vivo half-life by conjugation to an in vivo FcRn receptor. In some embodiments, such functions can be reduced or altered in an Fc for use with the provided Fc fusion proteins.

In some embodiments, one or more amino acid modifications may be introduced into the Fc region, thereby generating an Fc region variant. In some embodiments, the Fc region variant has decreased effector function. There are many examples of changes or mutations to Fc sequences that can alter effector function. For example, WO 00/42072, WO2006019447, WO2012125850, WO2015/107026, US2016/0017041 and Shields et al. J Biol. Chem. 9(2): 6591-6604 (2001) describe exemplary Fc variants with improved or diminished binding to FcRs. The contents of those publications are specifically incorporated herein by reference.

In some embodiments, the provided immunomodulatory proteins comprise an Fc region that exhibits reduced effector functions, which makes it a desirable candidate for applications in which the half-life of the immunomodulatory protein in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the immunomodulatory protein lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the immunomodulatory protein is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Immunomodulatory proteins with reduced effector function include those with substitution of one or more of Fc region residues 238, 265, 269, 270, 297, 327 and 329 by EU numbering (U.S. Pat. No. 6,737,056). Such Fc mutants include Fc mutants with substitutions at two or more of amino acid positions 265, 269, 270, 297 and 327 by EU numbering, including the so-called “DANA” Fc mutant with substitution of residues 265 and 297 to alanine (U.S. Pat. No. 7,332,581).

In some embodiments, the Fc region of immunomodulatory proteins has an Fc region in which any one or more of amino acids at positions 234, 235, 236, 237, 238, 239, 270, 297, 298, 325, and 329 (indicated by EU numbering) are substituted with different amino acids compared to the native Fc region. Such alterations of Fc region include, for example, alterations such as deglycosylated chains (N297A and N297Q), IgG1-N297G, IgG1-L234A/L235A, IgG1-L234A/L235E/G237A, IgG1-A325A/A330S/P331S, IgG1-C226S/C229S, IgG1-C226S/C229S/E233P/L234V/L235A, IgG1−E233P/L234V/L235A/G236del/S267K, IgG1-L234F/L235E/P331S, IgG1-S267E/L328F, IgG2-V234A/G237A, IgG2-H268Q/V309L/A330S/A331S, IgG4-L235A/G237A/E318A, and IgG4-L236E described in Current Opinion in Biotechnology (2009) 20 (6), 685-691; alterations such as G236R/L328R, L235G/G236R, N325A/L328R, and N325LL328R described in WO 2008/092117; amino acid insertions at positions 233, 234, 235, and 237 (indicated by EU numbering); and alterations at the sites described in WO 2000/042072.

Certain Fc variants with improved or diminished binding to FcRs are described. (See, e.g., U.S. Pat. No. 6,737,056; WO 2004/056312, WO2006019447 and Shields et al., J. Biol. Chem. 9(2): 6591-6604 (2001).)

In some embodiments, there is provided an immunomodulatory protein comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.) or WO2015107026. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434 by EU numbering, e.g., substitution of Fc region residue 434 (U.S. Pat. No. 7,371,826).

In some embodiments, the Fc region of the immunomodulatory protein comprises one or more amino acid substitutions C220S, C226S and/or C229S by EU numbering. In some embodiments, the Fc region of the immunomodulatory protein comprises one or more amino acid substitutions R292C and V302C. See also Duncan & Winter, Nature 322:738-40 (1988); U.S. Pat. Nos. 5,648,260; 5,624,821; and WO 94/29351 concerning other examples of Fc region variants.

In some embodiments, alterations are made in the Fc region that result in diminished C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, the variant Fc region comprising the one or more amino acid modifications (e.g. amino acid substitutions) is derived from a wild-type IgG1, such as a wild-type human IgG1. In some embodiments, the wild-type IgG1 Fc can be the Fc set forth in SEQ ID NO: 71 having an allotype containing residues Glu (E) and Met (M) at positions 356 and 358 by EU numbering. In some embodiments, the variant Fc region is derived from the amino acid sequence set forth in SEQ ID NO: 71. In other embodiments, the wild-type IgG1 Fc contains amino acids of the human G1m1 allotype, such as residues containing Asp (D) and Leu (L) at positions 356 and 358, e.g. as set forth in SEQ ID NO:81. Thus, in some cases, the variant Fc is derived from the amino acid sequence set forth in SEQ ID NO:81.

In some embodiments, the Fc region lacks the C-terminal lysine corresponding to position 232 of the wild-type or unmodified Fc set forth in SEQ ID NO: 71 or 81 (corresponding to K447del by EU numbering).

In some embodiments, the variant Fc region comprises a C5S amino acid modification of the wild-type or unmodified Fc region by numbering of SEQ ID NO: 71 (corresponding to C220S by EU numbering).

In some embodiments, the Fc region is a variant Fc that contains at least one amino acid substitution that is N82G by numbering of SEQ ID NO: 71 (corresponding to N297G by EU numbering). In some embodiments, the Fc further contains at least one amino acid substitution that is R77C or V87C by numbering of SEQ ID NO: 71 (corresponding to R292C or V302C by EU numbering). In some embodiments, the variant Fc region further comprises a C5S amino acid modification by numbering of SEQ ID NO: 71 (corresponding to C220S by EU numbering). For example, in some embodiments, the variant Fc region comprises the following amino acid modifications: N297G and one or more of the following amino acid modifications C220S, R292C or V302C by EU numbering (corresponding to N82G and one or more of the following amino acid modifications C5S, R77C or V87C with reference to SEQ ID NO:71), e.g., the Fc region comprises the sequence set forth in SEQ ID NO:82.

In some embodiments, the variant Fc contains the amino acid substitutions L234A/L235E/G237A, by EU numbering. In some embodiments, the variant Fc contains the amino acid substitutions A330S/P331S, by EU numbering. In some embodiments, the variant Fc contains the amino acid substitutions L234A/L235E/G237A/A330S/P331S (Gross et al. (2001) Immunity 15:289). In some embodiments, the variant Fc comprises the sequence set forth in SEQ ID NO: 175. In some embodiments, the variant Fc comprises the sequence set forth in SEQ ID NO:176. In some embodiments, an Fc region used in a construct provided herein can further lack a C-terminal lysine residue.

In some embodiments, the Fc region is a variant Fc that includes mutations L234A, L235E and G237A by EU numbering. In some embodiments, a wild-type Fc is further modified by the removal of one or more cysteine residue, such as by replacement of the cysteine residues to a serine residue at position 220 (C220S) by EU numbering. Exemplary inert Fc regions having reduced effector function are set forth in SEQ ID NO: 83 and SEQ ID NO:75, which are based on allotypes set forth in SEQ ID NO:71 or SEQ ID NO: 81, respectively. In some embodiments, an Fc region can further lack a C-terminal lysine residue. In some embodiments, the variant Fc region comprises one or more of the amino acid modifications C220S, L234A, L235E or G237A, e.g. the Fc region comprises the sequence set forth in SEQ ID NO:73, 75, 83 or 136. In some embodiments, the variant Fc comprises has the sequence set forth in SEQ ID NO: 73. In some embodiments, the variant Fc comprises has the sequence set forth in SEQ ID NO: 75. In some embodiments, the variant Fc comprises has the sequence set forth in SEQ ID NO: 83. In some embodiments, the variant Fc comprises has the sequence set forth in SEQ ID NO: 136.

In some embodiments, the Fc region is a variant Fc that has the sequence set forth in SEQ ID NO:73.

(SEQ ID NO: 73)
EPKSSDKTHTCPPCPAPEAEGAPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the Fc region is an IgG1 Fc but does not contain a hinge sequence. In some embodiments, the IgG1 Fc region does not contain the hinge sequence EPKSC (SEQ ID NO:239). In some embodiments, the IgG1 Fc region does not contain a hinge sequence EPKSS (SEQ ID NO: 238).

In some embodiments, the Fc region is a variant Fc that has the sequence set forth in SEQ ID NO: 221.

(SEQ ID NO: 221)
DKTHTCPPCPAPEAEGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG

In some embodiments, the Fc region is a variant Fc region that comprises one or more of the amino acid modifications C220S, E233P, L234V, L235A, G236del or S267K, e.g. the Fc region comprises the sequence set forth in SEQ ID NO:134. In some embodiments, the Fc region lacks the C-terminal lysine corresponding to position 232 of the wild-type or unmodified Fc set forth in SEQ ID NO: 71 (corresponding to K447del by EU numbering). In some embodiments, the Fc region comprises the sequence set forth in SEQ ID NO:137.

In some embodiments, the Fc region is a variant Fc region that comprises one or more of the amino acid modifications C220S, R292C, N297G, V302C. In some embodiments, the Fc region lacks the C-terminal lysine corresponding to position 232 of the wild-type or unmodified Fc set forth in SEQ ID NO: 71 (corresponding to K447del by EU numbering). An exemplary variant Fc region is set forth in SEQ ID NO: 135.

In some embodiments, the variant Fc region comprises one or more of the amino acid modifications C220S/E233P/L234V/L235A/G236del/S267K. In some embodiments, the Fc region lacks the C-terminal lysine corresponding to position 232 of the wild-type or unmodified Fc set forth in SEQ ID NO: 71 (corresponding to K447del by EU numbering). An exemplary variant Fc region is set forth in SEQ ID NO: 137.

Examples of such Fc regions for inclusion in an immunomodulatory polypeptide are set forth in Table 2.

TABLE 2
Exemplary IgG1 Fc Regions, wild-type or variant (effectorless)

| | 356E/358M | 356D/358L |
|---|---|---|
| | allotype | allotype |
| Fc mutations (EU numbering) | SEQ ID NO | SEQ ID NO |
| (wild-type) | 71 | 81 (with |
| | | C220S, |
| | | K447del) |
| C220S, R292C, N297G, V302C | 82 | |
| C220S, R292C, N297G, V302C, | 135 | |
| K447del | | |
| C220S, L234A, L235E, G237A | 83 | 75 |
| C220S, L234A, L235E, G237A, | 136 | 73 |
| K447del | | |
| L234A, L235E, G237A, K447del, | | 221 |
| with deletion of hinge | | |
| C220S, E233P, L234V, L235A, | 134 | |
| G236del, S267K | | |
| C220S/E233P/L234V/L235A/ | 137 | |
| G236del/S267K/K447del | | |
| L234A, L235E, G237A, A330S, | | 176 |
| P331S | | |
| L234A, L235E, G237A, A330S, | | 175 |
| P331S, with deletion of hinge | | |

In some embodiments, the Fc region is a variant Fc region containing any combination of the Fc mutations in Table 2. In some embodiments, the Fc region is a variant Fc region having the sequence set forth in any one of the SEQ ID NOs in Table 2.

For example, a variant Fc region may be an effectorless Fc that exhibits reduced effector activity compared to a wild-type IgG1 set forth in SEQ ID NO:71 or SEQ ID NO:81. In some embodiments, the variant Fc comprises the sequence of amino acids set forth in any of SEQ ID NOS:75, 82, 83, 134, 73, 135, 136, or 137 or a sequence of amino acids that exhibits at least about 85%, 86%, 87%, 88%, 89%, 90%, 91%, 92%, 93%, 94%, 95%, 96%, 97%, 98%, 99% or more sequence identity to any of SEQ ID NOS: 75, 82, 83, 134, 73, 135, 136, or 137. In some embodiments, the variant Fc has the sequence set forth in SEQ ID NO: 73. In embodiments, when produced and expressed from cells, the provided immunomodulatory protein (e.g. TACI-Fc fusion) is a homodimer containing two identical polypeptide chains.

In some embodiments, the immunomodulatory protein contains a first immunomodulatory Fc fusion polypeptide and a second immunomodulatory Fc fusion polypeptide in which the first and second polypeptide are different. In some embodiments, a first Fc polypeptide fusion contains an Fc region and one or more variant TACI polypeptide sequence and a second polypeptide fusion contains an Fc region and one or more TACI polypeptide sequence. In such embodiments, the Fc region can be a region that promotes or facilitates formation of heterodimers.

In some embodiments, the Fc domain of one or both of the first and second immunomodulatory Fc fusion polypeptides comprise a modification (e.g. substitution) such that the interface of the Fc molecule is modified to facilitate and/or promote heterodimerization. Methods to promote heterodimerization of Fc chains include mutagenesis of the Fc region, such as by including a set of “knob-into-hole” mutations or including mutations to effect electrostatic steering of the Fc to favor attractive interactions among different polypeptide chains. In some embodiments, the Fc region of the heterodimeric molecule additionally can contain one or more other Fc mutation, such as any described above. In some embodiments, the heterodimer molecule contains an Fc region with a mutation that reduces effector function. In some embodiments, such Fc regions contain mutations C220S, L234A, L235E and/or G237A by EU numbering. In some embodiments, any of the above mutations in an Fc backbone can be made in an allotype containing residues Glu (E) and Met (M) at positions 356 and 358 by EU numbering. In other embodiments, any of the above mutations in an Fc backbone can be made in an allotype containing residue Asp (D) and Leu (L) at positions 356 and 358 by EU numbering.

In some embodiments, modifications include introduction of a protuberance (knob) into a first Fc polypeptide and a cavity (hole) into a second Fc polypeptide such that the protuberance is positionable in the cavity to promote complexing of the first and second Fc-containing polypeptides. Amino acids targeted for replacement and/or modification to create protuberances or cavities in a polypeptide are typically interface amino acids that interact or contact with one or more amino acids in the interface of a second polypeptide.

In some embodiments, a first polypeptide that is modified to contain protuberance (knob) amino acids include replacement of a native or original amino acid with an amino acid that has at least one side chain which projects from the interface of the first polypeptide and is therefore positionable in a compensatory cavity (hole) in an adjacent interface of a second polypeptide. Most often, the replacement amino acid is one which has a larger side chain volume than the original amino acid residue. One of skill in the art knows how to determine and/or assess the properties of amino acid residues to identify those that are ideal replacement amino acids to create a protuberance. In some embodiments, the replacement residues for the formation of a protuberance are naturally occurring amino acid residues and include, for example, arginine (R), phenylalanine (F), tyrosine (Y), or tryptophan (W). In some examples, the original residue identified for replacement is an amino acid residue that has a small side chain such as, for example, alanine, asparagine, aspartic acid, glycine, serine, threonine, or valine.

In some embodiments, a second polypeptide that is modified to contain a cavity (hole) is one that includes replacement of a native or original amino acid with an amino acid that has at least one side chain that is recessed from the interface of the second polypeptide and thus is able to accommodate a corresponding protuberance from the interface of a first polypeptide. Most often, the replacement amino acid is one which has a smaller side chain volume than the original amino acid residue. One of skill in the art knows how to determine and/or assess the properties of amino acid residues to identify those that are ideal replacement residues for the formation of a cavity. Generally, the replacement residues for the formation of a cavity are naturally occurring amino acids and include, for example, alanine (A), serine (S), threonine (T) and valine (V). In some examples, the original amino acid identified for replacement is an amino acid that has a large side chain such as, for example, tyrosine, arginine, phenylalanine, or tryptophan.

The CH3 interface of human IgG1, for example, involves sixteen residues on each domain located on four anti-parallel β-strands which buries 1090 Å2 from each surface (see e.g., Deisenhofer et al. (1981) Biochemistry, 20:2361-2370; Miller et al., (1990) J Mol. Biol., 216, 965-973; Ridgway et al., (1996) Prot. Engin., 9: 617-621; U.S. Pat. No. 5,731,168). Modifications of a CH3 domain to create protuberances or cavities are described, for example, in U.S. Pat. No. 5,731,168; International Patent Applications WO98/50431 and WO 2005/063816; and Ridgway et al., (1996) Prot. Engin., 9: 617-621. In some examples, modifications of a CH3 domain to create protuberances or cavities are typically targeted to residues located on the two central anti-parallel β-strands. The aim is to minimize the risk that the protuberances which are created can be accommodated by protruding into the surrounding solvent rather than being accommodated by a compensatory cavity in the partner CH3 domain.

In some embodiments, the heterodimeric molecule contains a T366W mutation in the CH3 domain of the “knobs chain” and T366S, L368A, Y407V mutations in the CH3 domain of the “hole chain”. In some cases, an additional interchain disulfide bridge between the CH3 domains can also be used (Merchant, A. M., et al., Nature Biotech. 16 (1998) 677-681) e.g. by introducing a Y349C mutation into the CH3 domain of the “knobs” or “hole” chain and a E356C mutation or a S354C mutation into the CH3 domain of the other chain. In some embodiments, the heterodimeric molecule contains S354C, T366W mutations in one of the two CH3 domains and Y349C, T366S, L368A, Y407V mutations in the other of the two CH3 domains. For example, the knob Fc may contain the sequence set forth in SEQ ID NO: 89, containing S354C and T366W, and a hole Fc set forth in SEQ ID NO: 90, containing mutations Y349C, T366S, L368A and Y407V). In some embodiments, the heterodimeric molecule comprises E356C, T366W mutations in one of the two CH3 domains and Y349C, T366S, L368A, Y407V mutations in the other of the two CH3 domains. In some embodiments, the heterodimeric molecule comprises Y349C, T366W mutations in one of the two CH3 domains and E356C, T366S, L368A, Y407V mutations in the other of the two CH3 domains. In some embodiments, the heterodimeric molecule comprises Y349C, T366W mutations in one of the two CH3 domains and S354C, T366S, L368A, Y407V mutations in the other of the two CH3 domains. Examples of other knobs-in-holes technologies are known in the art, e.g. as described by EP 1 870 459 A1.

In some embodiments, an Fc variant containing CH3 protuberance (knob) or cavity (hole) modifications can be joined to a multi-domain immunomodulatory polypeptide anywhere, but typically via its N- or C-terminus, to the N- or C-terminus of the one or more TACI polypeptide sequence (e.g. variant TACI polypeptide sequence), such as to form a fusion polypeptide. The linkage can be direct or indirect via a linker. Typically, a knob and hole molecule is generated by co-expression of a first immunomodulatory polypeptide linked to an Fc variant containing CH3 protuberance modification(s) with a second immunomodulatory polypeptide linked to an Fc variant containing CH3 cavity modification(s).

Exemplary sequences for knob and hole Fc polypeptides are set forth in SEQ ID NOs: 128, and 129, respectively. In some embodiments, the knob or hold Fc region lacks the C-terminal lysine corresponding to position 232 of the wild-type or unmodified Fc set forth in SEQ ID NO: 71 (corresponding to K447del by EU numbering). Exemplary sequences for knob and hole Fc polypeptides are set forth in SEQ ID NOs: 89 and 90, respectively.

In some embodiment, individual polypeptide of a multi-domain polypeptide or individual polypeptides of a single-domain polypeptide are linked to a multimerization domain that forms an immunomodulatory protein is a trimer, tetramer or pentamer. In some embodiments, the individual polypeptides of such a molecule are the same. In some embodiments, such a multimerization domain is a cartilage oligomeric matrix protein (COMP) assembly domain, a vasodilator-stimulated phosphoprotein (VASP) tetramerization domain or a ZymoZipper (ZZ) 12.6 domain.

In some embodiments, the multimerization domain is a portion of the cartilage oligomeric matrix protein (COMP) assembly domain (Voulgaraki et al., Immunology (2005) 115(3):337-346. In some examples, the COMP is or contains an amino acid sequence as set forth in SEQ ID NO: 146 (e.g. amino acids 29-72 of the full length COMP, Uniprot accession number P49747) or a sequence that has about 85%, 85%, 87%, 88%, 89%, 90%, 91%, 92%, 93%, 94%, 95%, 96%, 97%, 98%, 99% or more sequence identity to SEQ ID NO: 146.

In some embodiments, the multimerization domain is a vasodilator-stimulated phosphoprotein (VASP) tetramerization domain (Bachmann et al., J Biol Chem (1999) 274(33):23549-23557). In some embodiments, the VASP is or contains an amino acid sequence as set forth in SEQ ID NO: 147 (e.g. amino acids 343-375 of the full length VASP; Uniprot accession number P50552) or a sequence that has about 85%, 85%, 87%, 88%, 89%, 90%, 91%, 92%, 93%, 94%, 95%, 96%, 97%, 98%, 99% or more sequence identity to SEQ ID NO: 147.

In some embodiments, a TACI polypeptide sequence (e.g. variant TACI polypeptide sequence) is joined to the multimerization domain (e.g. Fc region) via a linker, such as a peptide linker. In some embodiments, a peptide linker can be a single amino acid residue or greater in length. In some embodiments, the peptide linker has at least one amino acid residue but is no more than 20, 19, 18, 17, 16, 15, 14, 13, 12, 11, 10, 9, 8, 7, 6, 5, 4, 3, 2, or 1 amino acid residues in length.

In some embodiments, the linker is (in one-letter amino acid code): GGGGS (“4GS”; SEQ ID NO: 77) or multimers of the 4GS linker, such as repeats of 2, 3, 4, or 5 4GS linkers. In some embodiments, the peptide linker is the peptide linker is (GGGGS)₂ (SEQ ID NO: 78), (GGGGS)₃ (SEQ ID NO: 79), (GGGGS)₄ (SEQ ID NO: 84) or (GGGGS)₅ (SEQ ID NO: 91). In some embodiments, the linker also can include a series of alanine residues alone or in addition to another peptide linker (such as a 4GS linker or multimer thereof). In some embodiments, the linker (in one-letter amino acid code) is GSGGGGS (SEQ ID NO: 74) or GGGGSSA (SEQ ID NO: 80). In some examples, the linker is a 2×GGGGS followed by three alanines (GGGGSGGGGSAAA; SEQ ID NO:133). In some examples, the linker is set forth in SEQ ID NO: 194 or 195.

In some embodiments, the TACI polypeptide, such as the variant TACI polypeptide, is directly linked to the Fc sequence. In some embodiments, the TACI polypeptide, such as the variant TACI polypeptide, is indirectly linked to the Fc sequence, such as via a linker. In some embodiments, one or more “peptide linkers” link the TACI polypeptide (e.g. variant TACI polypeptide) and the Fc region. In some embodiments, a peptide linker can be a single amino acid residue or greater in length. In some embodiments, the peptide linker has at least one amino acid residue but is no more than 20, 19, 18, 17, 16, 15, 14, 13, 12, 11, 10, 9, 8, 7, 6, 5, 4, 3, 2, or 1 amino acid residues in length. Exemplary linkers include any linker as described herein.

In some embodiments, the TACI-Fc fusion protein has the structure TACI polypeptide (TACI)-Linker-Fc region. In some embodiments, the immunomodulatory protein is a homodimer of two identical copies of the TACI-Fc fusion protein. For instance, interactions between Fc regions of the two identical polypeptide fusions form covalent disulfide bonds to result in a dimeric molecule containing two TACI polypeptides (e.g. two variant TACI polypeptides).

In some embodiments, there is provided a TACI-Fc fusion protein containing in order a TACI polypeptide, e.g. any as described above, a linker and an Fc region. In some embodiments, each TACI polypeptide of the TACI Fc fusion is a truncated wild-type TACI polypeptide, such as any as described. In some embodiments, the TACI polypeptide of the TACI Fc fusion is set forth in SEQ ID NO: 13. The linker may be any as described. In some embodiments, the linker is GSGGGGS (SEQ ID NO: 74). In some embodiments, the linker is GS(G4S)₂ (SEQ ID NO: 194). The Fc region may be any Fc region as described. In some embodiments, the Fc region is a wild-type IgG1 Fc set forth in SEQ ID NO:81. In some embodiments, the Fc region is a variant Fc set forth in SEQ ID NO: 73.

In some embodiments, the TACI-Fc fusion protein has the sequence set forth in SEQ ID NO:171. In some embodiments, the TACI-Fc fusion protein has the sequence set forth in SEQ ID NO:197. In some embodiments, the TACI-Fc fusion is encoded by the sequence set forth in SEQ ID NO:208.

(SEQ ID NO: 171)
SLSCRKEQGKFYDHLLRDCISCASICGQHPKQCAYFCENKLRSGSGGGGS
EPKSSDKTHTCPPCPAPEAEGAPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the TACI-Fc fusion protein has the sequence set forth in SEQ ID NO:172.

(SEQ ID NO: 172)
SLSCRKEQGKFYDHLLRDCISCASICGQHPKQCAYFCENKLRSGSGGGGS
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the TACI-Fc fusion protein has the sequence set forth in SEQ ID NO: 196, and encoded the sequence set forth in SEQ ID NO:207.

In some embodiments, the TACI polypeptide is a variant TACI polypeptide. In some embodiments, there is provided a variant TACI-Fc fusion protein containing in order a variant TACI polypeptide, e.g. any as described above, a linker and an Fc region. In some embodiments, the TACI polypeptide of the TACI Fc fusion is a variant TACI polypeptide, such as any as described. In some embodiments, the variant TACI of the variant TACI Fc fusion is set forth in any one of SEQ ID NOS: 2-12, 21, 22, or 101-120. In some embodiments, the variant TACI of the variant TACI Fc fusion is set forth in any one of SEQ ID NOS: 14-20, 23-35, 92-100 or 177-192. In some embodiments, the linker is GSGGGGS (SEQ ID NO: 74). In some embodiments, the linker is GS(G4S)₂ (SEQ ID NO: 194). In some embodiments, the Fc region is a wild-type IgG1 Fc set forth in SEQ ID NO:81. In some embodiments, the Fc region is a variant Fc set forth in SEQ ID NO: 73.

In some embodiments, the TACI-Fc fusion protein has the sequence of amino acids set forth in any one of SEQ ID NOS: 167-170, 200, or 222-237.

In some embodiments, the TACI-Fc fusion protein has the sequence set forth in SEQ ID NO:167.

(SEQ ID NO: 167)
SLSCRKEQGEYYDHLLRDCISCASICGQHPKQCADFCENKLRSGSGGGGS
EPKSSDKTHTCPPCPAPEAEGAPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the TACI-Fc fusion is encoded by the sequence set forth in SEQ ID NO:211.

In some embodiments, the TACI-Fc fusion protein has the sequence set forth in SEQ ID NO:168.

(SEQ ID NO: 168)
SLSCRKEQGEYYDHLLRDCISCASICGQHPKQCADFCENKLRSGSGGGGS
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the TACI-Fc fusion protein has the sequence set forth in SEQ ID NO: 169.

(SEQ ID NO: 169)
SLSCRKEEGKFYDHLLQDCISCASICGQHPKQCAYFCENKLRSGSGGGGS
EPKSSDKTHTCPPCPAPEAEGAPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the TACI-Fc fusion protein has the sequence set forth in SEQ ID NO:170

(SEQ ID NO: 170)
SLSCRKEEGKFYDHLLQDCISCASICGQHPKQCAYFCENKLRSGSGGGGS
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

In some embodiments, the TACI-Fc fusion protein contains multiple copies of a TACI polypeptide sequence (e.g. variant TACI-polypeptide sequence), such as 2, 3 or 4 TACI polypeptide sequences. In some embodiments, the TACI-Fc fusion proteins contain two TACI polypeptide sequences (e.g. two variant TACI polypeptide sequences). In some cases, the TACI polypeptide sequences may be linked directly or may be linked indirectly via a linker, such as a peptide linker including any as described. In such an example, one of the TACI polypeptide sequence is joined or linked to the Fc region, such as either to the N- or C-terminus of the Fc region. In other cases, the TACI polypeptide sequences may be separated from each other by the Fc region and each joined individually to the N- or C-terminus of the Fc region. The linkage to the Fc region may be direct or may be indirect via a linker, such as a peptide linker including any as described.

In some embodiments, the TACI polypeptide sequences (e.g. variant TACI polypeptide sequences) may be arranged in order in the fusion protein in tandem (hereinafter called a “tandem” Fc fusion construct). In some embodiments, the TACI-Fc fusion protein has the structure: (TACI)-Linker-(TACI)-Linker-Fc region. In some embodiments, the immunomodulatory protein is a tetravalent molecule that is a homodimer of two identical copies of the TACI-Fc fusion protein. For instance, interactions between Fc regions of the two identical polypeptide fusions form covalent disulfide bonds to result in a dimeric molecule containing four TACI polypeptides (e.g. four variant TACI polypeptides).

In some embodiments, there is provided a TACI-Fc fusion protein containing in order a TACI polypeptide, e.g. any as described above; a linker; another TACI polypeptide, e.g. any as described; and an Fc region. In some embodiments, each TACI polypeptide of the TACI Fc fusion is a truncated wild-type TACI polypeptide, such as any as described. In some embodiments, each TACI polypeptide of the TACI Fc fusion is set forth in SEQ ID NO: 13. In some embodiments, each TACI polypeptide of the TACI Fc fusion is a variant TACI polypeptide, such as any as described. In some embodiments, each TACI polypeptide of the TACI Fc fusion is a variant TACI set forth in any one of SEQ ID NOS: 2-12, 21, 22, or 101-120. In some embodiments, each TACI polypeptide of the TACI Fc fusion is a variant TACI set forth in any one of SEQ ID NOS: 14-20, 23-35, 92-100 or 177-192. The linkers may be any as described. In some embodiments, the linker is GSGGGGS (SEQ ID NO: 74). The Fc region may be any Fc region as described. In some embodiments, the Fc region is a wild-type IgG1 Fc set forth in SEQ ID NO:81. In some embodiments, the Fc region is a variant Fc set forth in SEQ ID NO: 73. In some embodiments, the TACI-Fc fusion protein has the sequence set forth in SEQ ID NO:198, and encoded by a sequence set forth in SEQ ID NO:209.

In some embodiments, the TACI polypeptide sequences (e.g. variant TACI polypeptide sequences) may be separated in the fusion protein by the Fc region in which the Fc region is positioned between the two TACI polypeptide sequences (hereinafter called a “barbell” Fc fusion construct). In some embodiments, the TACI-Fc fusion protein has the structure: (TACI)-Linker-Fc region-Linker-(TACI). In some embodiments, the linkers may be the same or different. In some embodiments, the immunomodulatory protein is a tetravalent molecule that is a homodimer of two identical copies of the TACI-Fc fusion protein. For instance, interactions between Fc regions of the two identical polypeptide fusions form covalent disulfide bonds to result in a dimeric molecule containing four TACI polypeptides (e.g. four variant TACI polypeptides).

In some embodiments, there is provided a TACI-Fc fusion protein containing in order a TACI polypeptide, e.g. any as described above; a linker; an Fc region; a linker; and another TACI polypeptide, e.g. any as described. In some embodiments, each TACI polypeptide of the TACI Fc fusion is a truncated wild-type TACI polypeptide, such as any as described. In some embodiments, each TACI polypeptide of the TACI Fc fusion is set forth in SEQ ID NO: 13. In some embodiments, each TACI polypeptide of the TACI Fc fusion is a variant TACI polypeptide, such as any as described. In some embodiments, each TACI polypeptide of the TACI Fc fusion is a variant TACI set forth in any one of SEQ ID NOS: 2-12, 21, 22, or 101-120. In some embodiments, each TACI polypeptide of the TACI Fc fusion is a variant TACI set forth in any one of SEQ ID NOS: 14-20, 23-35, 92-100 or 177-192. The linkers may be any as described, and may be the same of different. In some embodiments, the first linker is GSGGGGS (SEQ ID NO: 74) and the second linker is (GGGGS)₄ (SEQ ID NO: 84). The Fc region may be any Fc region as described. In some embodiments, the Fc region is a wild-type IgG1 Fc set forth in SEQ ID NO:81. In some embodiments, the Fc region is a variant Fc set forth in SEQ ID NO: 73. In some embodiments, the TACI-Fc fusion protein has the sequence set forth in SEQ ID NO:201, and encoded by a sequence set forth in SEQ ID NO:212. In some embodiments, the TACI-Fc fusion protein has the sequence set forth in SEQ ID NO:202, and encoded by a sequence set forth in SEQ ID NO:213.

In some embodiments, there is a provided a TACI-Fc fusion protein that is a dimer formed by two identical TACI polypeptides (e.g. variant TACI polypeptide) as described linked to an Fc domain. In some embodiments, identical species (also referred to as copies) of any of the provided TACI-Fc fusion polypeptides, e.g. variant TACI-Fc fusion, will be dimerized to create a homodimer. In some embodiments, the dimer is a homodimer in which the two TACI-Fc polypeptides, e.g. variant TACI-Fc polypeptides, are the same. For generating a homodimeric Fc molecule, the Fc region is one that is capable of forming a homodimer with a matched Fc region by co-expression of the individual Fc regions in a cell. In some embodiments, dimerization is mediated by covalent disulfide bond(s) formed between the Fc regions of the polypeptide fusions.

Also provided are nucleic acid molecules encoding the immunomodulatory protein. In some embodiments, for production of immunomodulatory protein, a nucleic acid molecule encoding the immunomodulatory protein is inserted into an appropriate expression vector. The resulting immunomodulatory protein can be expressed in host cells transformed with the expression where assembly between Fc domains occurs by interchain disulfide bonds formed between the Fc moieties to yield dimeric, such as divalent, immunomodulatory proteins.

Also provided are nucleic acid molecules encoding the TACI-Fc fusion proteins, e.g. variant TACI-Fc fusion protein. In some embodiments, for production of an Fc fusion protein, a nucleic acid molecule encoding a TACI-Fc fusion protein, e.g. variant TACI-Fc fusion protein is inserted into an appropriate expression vector. The resulting TACI-Fc fusion protein, e.g. variant TACI-Fc fusion protein can be expressed in host cells transformed with the expression where assembly between Fc domains occurs by interchain disulfide bonds formed between the Fc moieties to yield dimeric, such as divalent, TACI-Fc fusion proteins. The resulting Fc fusion proteins can be easily purified by affinity chromatography over Protein A or Protein G columns. For the generation of heterodimers, additional steps for purification can be necessary. For example, where two nucleic acids encoding different immunomodulatory proteins are transformed into cells, the formation of heterodimers must be biochemically achieved since immunomodulatory protein carrying the Fc-domain will be expressed as disulfide-linked homodimers as well. Thus, homodimers can be reduced under conditions that favor the disruption of interchain disulfides, but do no effect intra-chain disulfides. In some cases, different immunomodulatory protein monomers are mixed in equimolar amounts and oxidized to form a mixture of homo- and heterodimers. The components of this mixture are separated by chromatographic techniques. Alternatively, the formation of this type of heterodimer can be biased by genetically engineering and expressing immunomodulatory proteins containing Fc fusion molecules that contain one or more TACI variants using knob-into-hole methods as described.

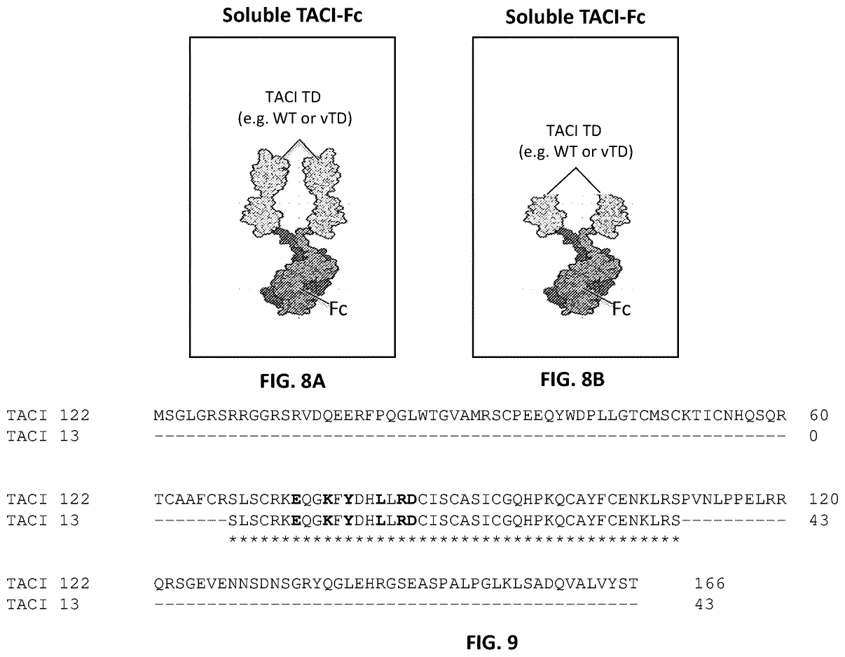

In embodiments, when produced and expressed from a cell, the provided immunomodulatory protein, such as a TACI-Fc (e.g. variant TACI-Fc), is a homodimer containing two identical polypeptide chains. FIG. 8A and FIG. 8B depict the structure of exemplary TACI-Fc fusion proteins provided herein.

Provided herein is a TACI (26)-Fc_73 homodimer of two identical variant TACI-Fc fusion proteins containing a variant of the TACI Cysteine Rich Domain 2 (CRD2) set forth in SEQ ID NO:26 designed to neutralize the B-cell stimulatory activity of APRIL and BAFF. The TACI (26)-Fc_73 homodimer is a dimer consisting of 2 identical receptor Fc-fusion protein chains, each with a variant TACI CRD2 domain human Fc-fusion set forth in SEQ ID NO:167, linked by covalent disulfide bonds.

Provided herein is a TACI (26)-Fc_81 homodimer of two identical variant TACI-Fc fusion proteins containing a variant of the TACI Cysteine Rich Domain 2 (CRD2) set forth in SEQ ID NO:26 designed to neutralize the B-cell stimulatory activity of APRIL and BAFF. The TACI (26)-Fc_81 homodimer is a dimer consisting of 2 identical receptor Fc-fusion protein chains, each with a variant TACI CRD2 domain human Fc-fusion set forth in SEQ ID NO:168, linked by covalent disulfide bonds.

Provided herein is a TACI (27)-Fc_73 homodimer of two identical variant TACI-Fc fusion proteins containing a variant of the TACI Cysteine Rich Domain 2 (CRD2) set forth in SEQ ID NO:27 designed to neutralize the B-cell stimulatory activity of APRIL and BAFF. The TACI (27)-Fc_73 homodimer is a dimer consisting of 2 identical receptor Fc-fusion protein chains, each with a variant TACI CRD2 domain human Fc-fusion set forth in SEQ ID NO:169, linked by covalent disulfide bonds.

Provided herein is a TACI (27)-Fc_81 homodimer of two identical variant TACI-Fc fusion proteins containing a variant of the TACI Cysteine Rich Domain 2 (CRD2) set forth in SEQ ID NO:27 designed to neutralize the B-cell stimulatory activity of APRIL and BAFF. The TACI (27)-Fc_81 homodimer is a dimer consisting of 2 identical receptor Fc-fusion protein chains, each with a variant TACI CRD2 domain human Fc-fusion set forth in SEQ ID NO:170, linked by covalent disulfide bonds.

In some embodiments, provided TACI-Fc (e.g. variant TACI-Fc) fusion proteins, such as homodimers thereof, exhibit an IC₅₀ for neutralizing BAFF of less than 400 pM. In some embodiments, the IC50 for neutralizing BAFF is between 1 pM and 400 pM, such as between 10 pM and 300 pM, between 10 pM and 200 pM, between 10 pM and 100 pM, between 10 pM and 50 pM, between 10 pM and 20 pM, between 20 pM and 400 pM, between 20 pM and 300 pM, between 20 pM and 200 pM, between 20 pM and 100 pM, between 20 pM and 50 pM, between 50 pM and 400 pM, between 50 pM and 300 pM, between 50 pM and 200 pM, between 50 pM and 100 pM, between 100 pM and 400 pM, between 100 pM and 300 pM, between 100 pM and 200 pM, between 200 pM and 400 pM, between 200 pM and 300 pM, or between 300 pM and 400 pM. In some embodiments, the IC₅₀for neutralizing BAFF is at or about 10 pM, 15 pM, 20 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM or 100 pM or any value between any of the foregoing.

In some embodiments, provided TACI-Fc (e.g. variant TACI-Fc) fusion proteins, such as homodimers thereof, exhibits an IC₅₀ for neutralizing APRIL of less than 400 pM. In some embodiments, the IC50 for neutralizing APRIL is between 0.5 pM and 100 pM, such as between 0.5 pM and 50 pM, between 0.5 pM and 25 pM, between 0.5 pM and 10 pM, between 0.5 pM and 5 pM, between 0.5 pM and 1 pM, between 1 pM and 100 pM, between 1 pM and 50 pM, between 1 pM and 25 pM, between 1 pM and 10 pM, between 1 pM and 5 pM, between 5 pM and 100 pM, between 5 pM and 50 pM, between 5 pM and 25 pM, between 5 pM and 10 pM, between 10 pM and 100 pM, between 10 pM and 50 pM, between 10 pM and 25 pM, or between 25 pM and 100 pM, between 25 pM and 50 pM, or between 50 pM and 100 pM. In some embodiments, the IC₅₀ for neutralizing APRIL is at or about 0.5 pM, 0.75 pM, 1 pM, 2 pM, 3 pM, 4 pM, 5 pM, 6 pM, 7 pM, 8 pM, 9 pM, 10 pM, 11 pM, 12 pM, 13 pM, 14 pM, 15 pM, 20 pM or 25 pM or any value between any of the foregoing.

### III. NUCLEIC ACIDS, VECTORS AND METHODS FOR PRODUCING THE POLYPEPTIDES OR CELLS

Provided herein are isolated or recombinant nucleic acids collectively referred to as “nucleic acids” which encode any of the immunomodulatory proteins provided herein. In some embodiments, nucleic acids provided herein, including all described below, are useful in recombinant production (e.g., expression) of immunomodulatory proteins provided herein. In some embodiments, nucleic acids provided herein, including all described below, are useful in expression of immunomodulatory proteins provided herein, such as TACI fusion proteins provided herein. The nucleic acids provided herein can be in the form of RNA or in the form of DNA, and include mRNA, cRNA, recombinant or synthetic RNA and DNA, and cDNA. The nucleic acids provided herein are typically DNA molecules, and usually double-stranded DNA molecules. However, single-stranded DNA, single-stranded RNA, double-stranded RNA, and hybrid DNA/RNA nucleic acids or combinations thereof comprising any of the nucleotide sequences of the invention also are provided.

In some cases, a heterologous (non-native) signal peptide can be added to the nucleic acid encoding the immunomodulatory protein. This may be desired, for example, in the case of expression of TACI fusion proteins, which do not contain an amino terminal signal sequence. In some embodiments, the signal peptide is a signal peptide from an immunoglobulin (such as IgG heavy chain or IgG-kappa light chain), a cytokine (such as interleukin-2 (IL-2), or CD33), a serum albumin protein (e.g. HSA or albumin), a human azurocidin preprotein signal sequence, a luciferase, a trypsinogen (e.g. chymotrypsinogen or trypsinogen) or other signal peptide able to efficiently express and, in some aspects, secret a protein from a cell. Exemplary signal peptides include any described in the Table 3.

TABLE 3
Exemplary Signal Peptides

| SEQ ID NO | Signal Peptide | Peptide Sequence |
|---|---|---|
| SEQ ID NO: 149 | HSA signal peptide | MKWVTFISLLFLFSSAYS |
| SEQ ID NO: 150 | Ig kappa light chain | MDMRAPAGIFGFLLVLFPGYRS |
| SEQ ID NO: 151 | human azurocidin preprotein | MTRLTVLALLAGLLASSRA |
| | signal sequence | |
| SEQ ID NO: 152 | IgG heavy chain signal peptide | MELGLSWIFLLAILKGVQC |
| SEQ ID NO: 153 | IgG heavy chain signal peptide | MELGLRWVFLVAILEGVQC |
| SEQ ID NO: 154 | IgG heavy chain signal peptide | MKHLWFFLLLVAAPRWVLS |
| SEQ ID NO: 155 | IgG heavy chain signal peptide | MDWTWRILFLVAAATGAHS |
| SEQ ID NO: 156 | IgG heavy chain signal peptide | MDWTWRFLFVVAAATGVQS |
| SEQ ID NO: 157 | IgG heavy chain signal peptide | MEFGLSWLFLVAILKGVQC |
| SEQ ID NO: 158 | IgG heavy chain signal peptide | MEFGLSWVFLVALFRGVQC |
| SEQ ID NO: 159 | IgG heavy chain signal peptide | MDLLHKNMKHLWFFLLLVAA |
| | | PRWVLS |
| SEQ ID NO: 160 | IgG Kappa light chain signal | MDMRVPAQLLGLLLLWLSGA |
| | sequences: | RC |
| SEQ ID NO: 161 | IgG Kappa light chain signal | MKYLLPTAAAGLLLLAAQPAM |
| | sequences: | A |
| SEQ ID NO: 162 | Gaussia luciferase | MGVKVLFALICIAVAEA |
| SEQ ID NO: 163 | Human albumin | MKWVTFISLLFLFSSAYS |
| SEQ ID NO: 164 | Human chymotrypsinogen | MAFLWLLSCWALLGTTFG |
| SEQ ID NO: 165 | Human interleukin-2 | MQLLSCIALILALV |
| SEQ ID NO: 166 | Human trypsinogen-2 | MNLLLILTFVAAAVA |

In some embodiments, the immunomodulatory protein comprises a signal peptide when expressed, and the signal peptide (or a portion thereof) is cleaved from the immunomodulatory protein upon secretion.

Also provided herein are recombinant expression vectors and recombinant host cells useful in producing the immunomodulatory proteins, such as TACI fusion proteins provided herein.

In any of the above provided embodiments, the nucleic acids encoding the immunomodulatory polypeptides provided herein can be introduced into cells using recombinant DNA and cloning techniques. To do so, a recombinant DNA molecule encoding an immunomodulatory polypeptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidite method. Also, a combination of these techniques could be used. In some instances, a recombinant or synthetic nucleic acid may be generated through polymerase chain reaction (PCR). A DNA insert encoding an immunomodulatory protein can be cloned into an appropriate transduction/transfection vector as is known to those of skill in the art. Also provided are expression vectors containing the nucleic acid molecules.

In some embodiments, the expression vectors are capable of expressing the immunomodulatory proteins in an appropriate cell under conditions suited to expression of the protein. In some aspects, nucleic acid molecule or an expression vector comprises the DNA molecule that encodes the immunomodulatory protein operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

In some embodiments, expression of the immunomodulatory protein is controlled by a promoter or enhancer to control or regulate expression. The promoter is operably linked to the portion of the nucleic acid molecule encoding the variant polypeptide or immunomodulatory protein.

The resulting recombinant expression vector having the DNA molecule thereon is used to transform an appropriate host. This transformation can be performed using methods well known in the art. In some embodiments, a nucleic acid provided herein further comprises nucleotide sequence that encodes a secretory or signal peptide operably linked to the nucleic acid encoding an immunomodulatory polypeptide such that a resultant soluble immunomodulatory polypeptide is recovered from the culture medium, host cell, or host cell periplasm. In other embodiments, the appropriate expression control signals are chosen to allow for membrane expression of an immunomodulatory polypeptide. Furthermore, commercially available kits as well as contract manufacturing companies can also be utilized to make engineered cells or recombinant host cells provided herein.

In some embodiments, the resulting expression vector having the DNA molecule thereon is used to transform, such as transduce, an appropriate cell. The introduction can be performed using methods well known in the art. Exemplary methods include those for transfer of nucleic acids encoding the receptors, including via viral, e.g., retroviral or lentiviral, transduction, transposons, and electroporation. In some embodiments, the expression vector is a viral vector. In some embodiments, the nucleic acid is transferred into cells by lentiviral or retroviral transduction methods.

Any of a large number of publicly available and well-known mammalian host cells, including mammalian T-cells or APCs, can be used in the preparing the polypeptides or engineered cells. The selection of a cell is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all cells can be equally effective for the expression of a particular DNA sequence.

In some embodiments, the host cell is a mammalian cell. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 (Chasin et al, Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40− transformed monkey kidney cells (COS-1; ATCC CRL 1650) and murine embryonic cells (NIH-3T3; ATCC CRL 1658).

In some embodiments, the host cells can be a variety of eukaryotic cells, such as in yeast cells, or with mammalian cells such as Chinese hamster ovary (CHO) or HEK293 cells. In some embodiments, the host cell is a suspension cell and the polypeptide is engineered or produced in cultured suspension, such as in cultured suspension CHO cells, e.g. CHO-S cells. In some examples, the cell line is a CHO cell line that is deficient in DHFR (DHFR−), such as DG44 and DUXB11. In some embodiments, the cell is deficient in glutamine synthase (GS), e.g. CHO-S cells, CHOK1 SV cells, and CHOZN((R)) GS−/− cells. In some embodiments, the CHO cells, such as suspension CHO cells, may be CHO-S-2H2 cells, CHO-S-clone 14 cells, or ExpiCHO-S cells.

In some embodiments, host cells can also be prokaryotic cells, such as with E. coli. The transformed recombinant host is cultured under polypeptide expressing conditions, and then purified to obtain a soluble protein. Recombinant host cells can be cultured under conventional fermentation conditions so that the desired polypeptides are expressed. Such fermentation conditions are well known in the art. Finally, the polypeptides provided herein can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, and affinity chromatography. Protein refolding steps can be used, as desired, in completing configuration of the mature protein. Finally, high performance liquid chromatography (HPLC) can be employed in the final purification steps.

In some embodiments, the recombinant vector is a viral vector. Exemplary recombinant viral vectors include a lentiviral vector genome, poxvirus vector genome, vaccinia virus vector genome, adenovirus vector genome, adenovirus-associated virus vector genome, herpes virus vector genome, and alpha virus vector genome. Viral vectors can be live, attenuated, replication conditional or replication deficient, non-pathogenic (defective), replication competent viral vector, and/or is modified to express a heterologous gene product, e.g., the variant immunomodulatory polypeptides provided herein. Vectors for generation of viruses also can be modified to alter attenuation of the virus, which includes any method of increasing or decreasing the transcriptional or translational load.

Exemplary viral vectors that can be used include modified vaccinia virus vectors (see, e.g., Guerra et al., J. Virol. 80:985-98 (2006); Tartaglia et al., AIDS Research and Human Retroviruses 8: 1445-47 (1992); Gheradi et al., J. Gen. Virol. 86:2925-36 (2005); Mayr et al., Infection 3:6-14 (1975); Hu et al., J. Virol. 75: 10300-308 (2001); U.S. Pat. Nos. 5,698,530, 6,998,252, 5,443,964, 7,247,615 and 7,368,116); adenovirus vector or adenovirus-associated virus vectors (see., e.g., Molin et al., J. Virol. 72:8358-61 (1998); Narumi et al., Am J. Respir. Cell Mol. Biol. 19:936-41 (1998); Mercier et al., Proc. Natl. Acad. Sci. USA 101:6188-93 (2004); U.S. Pat. Nos. 6,143,290; 6,596,535; 6,855,317; 6,936,257; 7,125,717; 7,378,087; 7,550,296); retroviral vectors including those based upon murine leukemia virus (MuLV), gibbon ape leukemia virus (GaLV), ecotropic retroviruses, simian immunodeficiency virus (SIV), human immunodeficiency virus (HIV), and combinations (see, e.g., Buchscher et al., J. Virol. 66:2731-39 (1992); Johann et al., J. Virol. 66: 1635-40 (1992); Sommerfelt et al., Virology 176:58-59 (1990); Wilson et al., J. Virol. 63:2374-78 (1989); Miller et al., J. Virol. 65:2220-24 (1991); Miller et al., Mol. Cell Biol. 10:4239 (1990); Kolberg, NIH Res. 4:43 1992; Cornetta et al., Hum. Gene Ther. 2:215 (1991)); lentiviral vectors including those based upon Human Immunodeficiency Virus (HIV-1), HIV-2, feline immunodeficiency virus (FIV), equine infectious anemia virus, Simian Immunodeficiency Virus (SIV), and maedi/visna virus (see, e.g., Pfeifer et al., Annu. Rev. Genomics Hum. Genet. 2: 177-211 (2001); Zufferey et al., J. Virol. 72: 9873, 1998; Miyoshi et al., J. Virol. 72:8150, 1998; Philpott and Thrasher, Human Gene Therapy 18:483, 2007; Engelman et al., J. Virol. 69: 2729, 1995; Nightingale et al., Mol. Therapy, 13: 1121, 2006; Brown et al., J. Virol. 73:9011 (1999); WO 2009/076524; WO 2012/141984; WO 2016/011083; McWilliams et al., J. Virol. 77: 11150, 2003; Powell et al., J. Virol. 70:5288, 1996) or any, variants thereof, and/or vectors that can be used to generate any of the viruses described above. In some embodiments, the recombinant vector can include regulatory sequences, such as promoter or enhancer sequences, that can regulate the expression of the viral genome, such as in the case for RNA viruses, in the packaging cell line (see, e.g., U.S. Pat. Nos. 5,385,839 and 5,168,062).

In some aspects, nucleic acids or an expression vector comprises a nucleic acid sequence that encodes the immunomodulatory protein operatively linked to appropriate expression control sequences. Methods of effecting this operative linking, either before or after the nucleic acid sequence encoding the immunomodulatory protein is inserted into the vector, are well known. Expression control sequences include promoters, activators, enhancers, operators, ribosomal binding sites, start signals, stop signals, cap signals, polyadenylation signals, and other signals involved with the control of transcription or translation. The promoter can be operably linked to the portion of the nucleic acid sequence encoding the immunomodulatory protein.

Transcriptional regulatory sequences include a promoter region sufficient to direct the initiation of RNA synthesis. Suitable eukaryotic promoters include the promoter of the mouse metallothionein I gene (Hamer et al, J. Molec. Appl Genet. 1:273 (1982)), the TK promoter of Herpes virus (McKnight, Cell 31:355 (1982)), the SV40 early promoter (Benoist et al, Nature 290:304 (1981)), the Rous sarcoma virus promoter (Gorman et al, Proc. Nat'l Acad. Sci. USA 79:6777 (1982)), the cytomegalovirus promoter (Foecking et al, Gene 45:101 (1980)), and the mouse mammary tumor virus promoter (see, generally, Etcheverry, “Expression of Engineered Proteins in Mammalian Cell Culture,” in Protein Engineering: Principles and Practice, Cleland et al. (eds.), pages 163-181 (John Wiley & Sons, Inc. 1996)). One useful combination of a promoter and enhancer is provided by a myeloproliferative sarcoma virus promoter and a human cytomegalovirus enhancer.

Alternatively, a prokaryotic promoter, such as the bacteriophage T3 RNA polymerase promoter, can be used to control production of an immunomodulatory protein in mammalian cells if the prokaryotic promoter is regulated by a eukaryotic promoter (Zhou et al, Mol Cell. Biol. 10:4529 (1990), and Kaufman et al, Nucl. Acids Res. 19:4485 (1991)).

An expression vector can be introduced into host cells using a variety of standard techniques including calcium phosphate transfection, liposome-mediated transfection, microprojectile-mediated delivery, electroporation, and the like. The transfected cells can be selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991).

For example, one suitable selectable marker is a gene that provides resistance to the antibiotic neomycin. In this case, selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A suitable amplifiable selectable marker is dihydrofolate reductase, which confers resistance to methotrexate. Other drug resistance genes (e.g., hygromycin resistance, multi-drug resistance, puromycin acetyltransferase) can also be used. Alternatively, markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

In some embodiments, polypeptides provided herein can also be made by synthetic methods. Solid phase synthesis is the preferred technique of making individual peptides since it is the most cost-effective method of making small peptides. For example, well known solid phase synthesis techniques include the use of protecting groups, linkers, and solid phase supports, as well as specific protection and deprotection reaction conditions, linker cleavage conditions, use of scavengers, and other aspects of solid phase peptide synthesis. Peptides can then be assembled into the polypeptides as provided herein.

### IV. PHARMACEUTICAL COMPOSITIONS

Provided herein are compositions containing any of the provided immunomodulatory proteins (e.g. TACI-Fc fusion protein) described herein. In some embodiments, the pharmaceutical compositions comprise a therapeutically effective amount of a TACI-Fc fusion protein as described provided as a formulation with a pharmaceutically acceptable diluent, carrier, solubilizer, emulsifier, preservative, and/or adjuvant. Also provided are any of the provided pharmaceutical compositions, including any of the provided formulations, for use in treating an autoimmune or inflammatory disease in a patient in need thereof, such as any uses for treating such diseases or conditions as described in Section VI. Also provided are methods of treating an autoimmune or inflammatory disease in a patient in need thereof by administering any of such pharmaceutical compositions or formulations, such as for treating any disease or conditions as described in Section VI.

The pharmaceutical composition can further comprise a pharmaceutically acceptable excipient. For example, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In some embodiments, the pharmaceutical composition is a solid, such as a powder, capsule, or tablet. For example, the components of the pharmaceutical composition can be lyophilized. In some embodiments, the solid pharmaceutical composition is reconstituted or dissolved in a liquid prior to administration.

In some embodiments, the pharmaceutical composition is a liquid, for example immunomodulatory proteins (e.g. TACI-Fc fusion protein) dissolved in an aqueous solution (such as physiological saline or Ringer's solution). In some embodiments, the pH of the pharmaceutical composition is between about 4.0 and about 8.5 (such as between about 4.0 and about 5.0, between about 4.5 and about 5.5, between about 5.0 and about 6.0, between about 5.5 and about 6.5, between about 6.0 and about 7.0, between about 6.5 and about 7.5, between about 7.0 and about 8.0, or between about 7.5 and about 8.5).

In some embodiments, the pharmaceutical composition comprises a pharmaceutically-acceptable excipient, for example a filler, binder, coating, preservative, lubricant, flavoring agent, sweetening agent, coloring agent, a solvent, a buffering agent, a chelating agent, or stabilizer. Examples of pharmaceutically-acceptable fillers include cellulose, dibasic calcium phosphate, calcium carbonate, microcrystalline cellulose, sucrose, lactose, glucose, mannitol, sorbitol, maltol, pregelatinized starch, corn starch, or potato starch. Examples of pharmaceutically-acceptable binders include polyvinylpyrrolidone, starch, lactose, xylitol, sorbitol, maltitol, gelatin, sucrose, polyethylene glycol, methyl cellulose, or cellulose. Examples of pharmaceutically-acceptable coatings include hydroxypropyl methylcellulose (HPMC), shellac, corn protein zein, or gelatin. Examples of pharmaceutically-acceptable disintegrants include polyvinylpyrrolidone, carboxymethyl cellulose, or sodium starch glycolate. Examples of pharmaceutically-acceptable lubricants include polyethylene glycol, magnesium stearate, or stearic acid. Examples of pharmaceutically-acceptable preservatives include methyl parabens, ethyl parabens, propyl paraben, benzoic acid, or sorbic acid. Examples of pharmaceutically-acceptable sweetening agents include sucrose, saccharine, aspartame, or sorbitol. Examples of pharmaceutically-acceptable buffering agents include carbonates, citrates, gluconates, acetates, phosphates, or tartrates.

In certain embodiments, the primary vehicle or carrier in a pharmaceutical composition may be either aqueous or non-aqueous in nature. For example, a suitable vehicle or carrier may be water for injection, physiological saline solution, or buffered saline. In some embodiments, pharmaceutical compositions comprise Tris buffer of about pH 7.0-8.5. In some embodiments, pharmaceutical composition comprises acetate buffer of about pH 4.0-6.0. The formulation can contain a concentration of buffer having sufficient buffering capacity to maintain a selected pH of the formulation at a selected temperature. In various embodiments, the concentration of the buffering solution can be from about 1 mM to about 100 mM, from about 2 mM to about 50 mM, from about 3 mM to about 30 mM, from about 4 mM to about 20 mM, or from about 5 mM to about 10 mM, or from about 10 mM to about 40 mM, from about 15 mM to about 35 mM, from about 20 mM to about 30 mM, from about 25 mM to about 35 mM about.

In some embodiments, the buffered solution contains acetate at a concentration of from about 1 mM to about 100 mM, from about 2 mM to about 50 mM, from about 3 mM to about 30 mM, from about 4 mM to about 20 mM, or from 5 mM to 15 mM, or from about 5 mM to about 10 mM, or from about 10 mM to about 40 mM, from about 15 mM to about 35 mM, from about 20 mM to about 30 mM, from about 25 mM to about 35 mM about. In some embodiments, the buffered solution contains acetate at a concentration from 5 mM to 15 mM. In some embodiments, the buffered solution contains acetate at a concentration of at or about 5 mM, at or about 6 mM, at or about 7 mM, at or about 8 mM, at or about 9 mM, at or about 10 mM, at or about 11 mM, at or about 12 mM, at or about 13 mM, at or about 14 mM, or at or about 15 mM, or any value between any of the foregoing. In some embodiments, the buffered solution contains acetate at a concentration of at or about 5 mM. In some embodiments, the buffered solution contains acetate at a concentration of at or about 10 nM. In some embodiments, the buffered solution contains acetate at a concentration of at or about 12 mM. In some embodiments, the buffered solution contains acetate at a concentration of at or about 15 mM. Exemplary pH ranges of an acetic acid (acetate) buffer and/or the final formulation can include pH ranges between about 4.0 to about 6.0, between about 4.5 to about 5.5, between about 4.8 to about 5.2 or about 5.0. Accordingly, an acetic acid (acetate) buffer and/or the final formulation can be prepared to have a pH of about 4.0, about 4.5, about 4.8, about 5.0, about 5.2, about 5.5, about 5.7, or about 6.0, or any value between any of the foregoing. In some embodiments, the pH of the buffered solution is at or about 5.0. In some embodiments, the pH of the buffered solution is at or about 5.2. In some embodiments, the pH of the buffered solution is at or about 5.5. Those skilled in the art can determine the pH of an acetic acid (acetate) buffer in a formulation.

In certain embodiments, acceptable formulation materials are nontoxic to recipients at the dosages and concentrations employed. In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as proline, glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18” Edition, (A. R. Genrmo, ed.), 1990, Mack Publishing Company.

Free amino acids, such as but not limited to, lysine, proline, serine, and alanine can be used for stabilizing proteins in a provided formulation as bulking agents, stabilizers, and antioxidants, as well as other standard uses. In some embodiments, the free amino acid is present in the formulation at a concentration of about 1% to about 10% or 2% to 5%.

In some embodiments, the formulation contains proline as a free amino acid. In some embodiments, provided formulations contain proline at a concentration of about 1% to about 10%. In some embodiments, provided formulations contain proline at a concentration of about 2% to about 5%. In some embodiments, provided formulations contain proline at a concentration of at or about 1%, at or bout 2%, at or about 3%, at or about 4%, at or about 5%, at or about 6%, at or about 7%, at or about 8%, at or about 9%, at or about 10%, or any value between any of the foregoing. In some embodiments, provided formulations contain proline at a concentration of about or about 2%. In some embodiments, provided formulations contain proline at a concentration of at or about 3%. In some embodiments, provided formulations contain proline at a concentration of at or about 4%.

Provided formulations may also further comprise surfactants. Protein molecules may be susceptible to adsorption on surfaces and to denaturation and consequent aggregation at air-liquid, solid-liquid, and liquid-liquid interfaces. These effects generally scale inversely with protein concentration. In some cases, the effects may be exacerbated by physical agitation, such as that generated during the shipping and handling of a product. Surfactants may be used to prevent, minimize, or reduce surface adsorption. A surfactant for inclusion in a formulation can be chosen, for example, to enhance or promote retention in stability of the protein molecule by preventing or reducing aggregation and/or adsorption. Sorbitan fatty acid esters such as the polysorbates are surfactants exhibiting a wide range of hydrophilic and emulsifying characteristics. They can be used individually or in combination with other surfactants to cover a wide range of stabilization needs. Such characteristics can be suitable for use with active protein agents because they can be tailored to cover the wide range of hydrophobic and hydrophilic characteristics of biopharmaceuticals. Useful surfactants include, but are not limited to, polysorbate 20, polysorbate 80, other fatty acid esters of sorbitan polyethoxylates, and poloxamer 188.

Surfactant concentration for provided formulations can be less than about 1% (w/v). In this regard, surfactant concentrations generally can be used at ranges between about 0.001-0.10% (w/v), between about 0.001-0.05% (w/v), between about 0.001-0.025% (w/v), between about 0.001-0.01% (w/v), between about 0.005-0.10%, between about 0.005-0.05%, between about 0.005-0.025%, between about 0.005-0.01%, between about 0.01%-0.10%, between about 0.01%-0.05%, or between about 0.01% to 0.025%. Surfactant concentrations and/or amounts less than, greater than or in between these ranges also can also be used. Accordingly, a formulation can be produced that contains essentially any desired concentration or amount of one or more surfactants including, for example, about 0.001% (w/v), 0.002% (w/v), 0.003% (w/v), 0.004% (w/v), 0.005% (w/v), 0.006% (w/v), 0.007% (w/v), 0.008% (w/v), 0.009% (w/v), 0.010% (w/v), 0.015% (w/v), 0.02% (w/v), 0.025% (w/v), 0.03% (w/v), 0.04% (w/v), 0.05% (w/v), 0.06% (w/v), 0.07% (w/v), 0.08% (w/v), 0.09% (w/v) or 0.10% (w/v), or any value between any of the foregoing.

In some embodiments, the surfactant is polysorbate 80. In some embodiments, polysorbate 80 is at a concentration in the formulation of between about 0.001-0.10% (w/v), between about 0.001-0.05% (w/v), between about 0.001-0.025% (w/v), between about 0.001-0.01% (w/v), between about 0.005-0.10%, between about 0.005-0.05%, between about 0.005-0.025%, between about 0.005-0.01%, between about 0.01%-0.10%, between about 0.01%-0.05%, or between about 0.01% to 0.025%. In some embodiments, polysorbate 80 is present at a concentration of 0.001% (w/v), 0.002% (w/v), 0.003% (w/v), 0.004% (w/v), 0.005% (w/v), 0.006% (w/v), 0.007% (w/v), 0.008% (w/v), 0.009% (w/v), 0.010% (w/v), 0.015% (w/v), 0.02% (w/v), 0.025% (w/v), 0.03% (w/v), 0.04% (w/v), 0.05% (w/v), 0.06% (w/v), 0.07% (w/v), 0.08% (w/v), 0.09% (w/v) or 0.10% (w/v), or any value between any of the foregoing. In some embodiments, the concentration of polysorbate 80 is at or about 0.010% (w/v). In some embodiments, the concentration of polysorbate 80 is at or about 0.015% (w/v). In some embodiments, the concentration of polysorbate 80 is at or about 0.02% (w/v).

In some embodiments, the pharmaceutical composition further comprises an agent for the controlled or sustained release of the product, such as injectable microspheres, bio-erodible particles, polymeric compounds (polylactic acid, polyglycolic acid), beads, or liposomes.

In some embodiments, the pharmaceutical composition is sterile. Sterilization may be accomplished by filtration through sterile filtration membranes or radiation. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

A pharmaceutically acceptable carrier may be a pharmaceutically acceptable material, composition, or vehicle. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

In some embodiments, the pharmaceutical composition is formulated to contain an amount of a TACI-Fc fusion of from at or about 1 mg to at or about 100 mg, such as from at or about 1 mg to at or about 75 mg, from at or about 1 mg to at or about 50 mg, from at or about 1 mg to at or about 25 mg, from at or about 1 mg to at or about 10 mg, from at or about 1 mg to at or about 5 mg, from at or about 5 mg to at or about 100 mg, from at or about 5 mg to at or about 75 mg, from at or about 5 mg to at or about 50 mg, from at or about 5 mg to at or about 25 mg, from at or about 5 mg to at or about 10 mg, from at or about 10 mg to at or about 100 mg, from at or about 10 mg to at or about 75 mg, from at or about 10 mg to at or about 50 mg, from at or about 10 mg to at or about 25 mg, from at or about 25 mg to at or about 100 mg, from at or about 25 mg to at or about 75 mg, from at or about 25 mg to at or about 50 mg, from at or about 50 mg to at or about 100 mg, from at or about 50 mg to at or about 75 mg or from at or about 75 mg to at or about 100 mg. In some embodiments, the pharmaceutical composition is formulated to contain an amount of a TACI-Fc fusion protein that is at or about 10 mg, at or about 20 mg, at or about 25 mg, at or about 30 mg, at or about 40 mg, at or about 50 mg, at or about 60 mg, at or about 70 mg, at or about 75 mg, at or about 80 mg or at or about 100 mg, or any value between any of the foregoing. In some embodiments, the pharmaceutical composition is formulated to contain an amount of a TACI-Fc fusion protein that is at or about 80 mg.

In some embodiments, the pharmaceutical composition is formulated in a volume that is from at or about 0.5 mL to at or about 10 mL, such as from at or about 0.5 mL to at or about 5 mL, from at or about 0.5 mL to at or about 2 mL, from at or about 0.5 mL to at or about 1 mL, from at or about 1 mL to at or about 10 mL, from at or about 1 mL to at or about 5 mL or from at or about 5 mL to at or about 10 mL. In some embodiments, the pharmaceutical composition is formulated in a volume that is at or about 0.5 mL, at or about 1 mL, at or about 2 mL, at or about 2.5 mL, at or about 3 mL, at or about 4 mL, at or about 5 mL, at or about 6 mL, at or about 7 mL, at or about 8 mL, at or about 9 mL or at or about 10 mL. In some embodiments, the composition is formulated in a volume that is at or about 0.5 mL, 0.6 mL, 0.7 mL, 0.8 mL, 0.9 mL, 1.0 mL, 1.2 mL, 1.4. mL, 1.6 mL, 1.8 mL or 2.0 mL, or any value between any of the foregoing.

In some embodiments, the concentration of the TACI-Fc fusion protein in the composition is from at or about 1 mg/mL to at or about 50 mg/mL, such as from at or about 1 mg/mL to at or about 25 mg/mL, from at or about 1 mg/mL to at or about 15 mg/mL, from at or about 1 mg mL to at or about 5 mg/mL, from at or about 5 mg/mL to at or about 50 mg/mL, from at or about 5 mg/mL to at or about 25 mg/mL, from at or about 5 mg/mL to at or about 15 mg/mL, from at or about 15 mg/mL to at or about 50 mg/mL, from at or about 15 mg/mL to at or about 25 mg/mL or from at or about 25 mg/mL to at or about 50 mg/mL. In some embodiments, the concentration of the TACI-Fc fusion protein in the composition is from at or about 1 mg/mL, at or about 5 mg/mL, at or about 10 mg/mL, at or about 15 mg/mL, at or about 20 mg/mL, at or about 25 mg/mL, at or about 30 mg/mL, at or about 40 mg/mL or at or about 50 mg/mL. Provided herein are any of such compositions contained in a container such as a vial. In particular aspects, the container, such as vial, is sterile. The container may be any biocompatible container, such as a glass container. In some embodiments, the vial is a 2 mL glass vial.

In some embodiments, the concentration of the TACI-Fc fusion protein in the composition is higher than 50 mg/mL. In some embodiments, the concentration of the composition is between at or about 50 mg/mL and 200 mg/mL, such as between at or about 50 mg/mL and 150 mg/mL, between at or about 50 mg/mL and 100 mg/mL, between at or about 100 mg/mL and 200 mg/mL, between at or about 100 mg/mL and 150 mg/mL or between at or about 150 mg/mL and 200 mg/mL. In some embodiments, the concentration of the TACI-Fc fusion protein in the composition is at or about 60 mg/mL, at or about 70 mg/mL, at or about 80 mg/mL, at or about 100 mg/mL, at or about 120 mg/mL, at or about 140 mg/mL, at or about 160 mg/mL, at or about 180 mg/mL or at or about 200 mg/mL, or any value between any of the foregoing. In some embodiments, the concentration of the TACI-Fc fusion protein in the composition is at or about 100 mg/mL. Provided herein are any of such compositions contained in a container such as a vial. In particular aspects, the container, such as vial, is sterile. The container may be any biocompatible container, such as a glass container. In some embodiments, the vial is a 2 mL glass vial.

In some embodiments, the TACI-Fc fusion protein, such as any described, is formulated in a buffered solution containing 10 mM Acetate, 3% proline, 0.015% polysorbate 80 at a pH of 5.2. In some embodiments, the TACI-Fc fusion protein is provided at 100/mg/mL as a liquid for injection (e.g. IV or SC). In some embodiments, the TACI-Fc fusion protein is provided in a volume of at or about 8 mL (e.g. 80 mg) in a container, such as in a 2 mL glass vial.

In some embodiments of the provided formulations, the TACI-Fc fusion protein is a homodimer of two polypeptide of the formula TACI-linker-Fc in which the TACI is a variant TACI that is a portion of the extracellular domain composed of the CRD2 TNF receptor domain set forth in SEQ ID NO:13 in which is present amino acid substitutions K77E, F78Y and Y102D. In some embodiments, the variant TACI is set forth in SEQ ID NO:26. In embodiments of any of the described TACI-Fc fusion proteins, the variant TACI is linked to the Fc domain via the linker. In some of any of the provided methods or uses, the TACI-Fc fusion protein has the sequence set forth in SEQ ID NO: 167. In some of any embodiments, the TACI-Fc fusion protein has the sequence set forth in SEQ ID NO: 168.

In some of any embodiments, when administering the TACI-Fc fusion protein or formulation containing the same at a concentration less than 100 mg/mL it may be diluted in a physiologically acceptable buffer, such as 0.9% sodium chloride (Normal saline).

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. Also provided herein are kits for producing a single-dose administration unit. In some aspects, the kits may each contain both a first container having a dried protein and a second container having an aqueous formulation. In certain embodiments kits containing single and multi-chambered pre-filled syringes (e.g., liquid syringes and lyosyringes) are provided.

In some embodiments, the pharmaceutical composition, such as any of the provided formulations, are stable at or about −20° C. for up to 6 months or more, such as for up to 12 months or more. In some embodiments, the pharmaceutical composition is stored at or about −20° C. In some embodiments, the storage is under conditions in which the formulation of the TACI-Fc fusion protein is protected from light.

In some embodiments, the pharmaceutical composition is administered to a subject. Generally, dosages and routes of administration of the pharmaceutical composition are determined according to the size and condition of the subject, according to standard pharmaceutical practice. For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy.

Long-acting pharmaceutical compositions may be administered every 3 to 4 days, every week, or biweekly depending on the half-life and clearance rate of the particular formulation. The frequency of dosing will depend upon the pharmacokinetic parameters of the molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally.

In some embodiments, a provided pharmaceutical formulation may, for example, be in a form suitable for intravenous infusion. In some embodiments, a provided formulation may be in in a form suitable for subcutaneous administration.

### V. METHODS FOR ASSESSING ACTIVITY AND IMMUNE MODULATION OF IMMUNOMODULATORY PROTEINS

In some embodiments, the provided immunomodulatory proteins, such as TACI fusion proteins provided herein exhibit immunomodulatory activity. The provided immunomodulatory proteins, such as TACI fusion protein can modulate B cell activity, such as one or more of B cell proliferation, differentiation or survival.

The function of immunomodulatory proteins can be examined using a variety of approaches to assess the ability of the proteins to bind to cognate binding partners. For example, TACI fusion proteins may be assessed for binding to APRIL or BAFF. A variety of assays are known for assessing binding affinity and/or determining whether a binding molecule (e.g., immunomodulatory protein) specifically binds to a particular binding partner. It is within the level of a skilled artisan to determine the binding affinity of a binding molecule, e.g., immumodulatory protein, for a binding partner, e.g., APRIL or BAFF, such as by using any of a number of binding assays that are well known in the art. Various binding assays are known and include, but are not limited to, for example, ELISA K_{D}, KinExA, flow cytometry, and/or surface plasmon resonance devices), including those described herein. Such methods include, but are not limited to, methods involving BIAcore®, Octet®, or flow cytometry. For example, in some embodiments, a BIAcore® instrument can be used to determine the binding kinetics and constants of a complex between two proteins using surface plasmon resonance (SPR) analysis (see, e.g., Scatchard et al., Ann. N. Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins.

Provided immunomodulatory proteins also can be assessed in any of a variety of assays to assess modulation of B cell activity. One such assay is a cell proliferation assay. Cells are cultured in the presence or absence of a test compound (e.g. immunomodulatory protein), and cell proliferation is detected by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, J. Immunol. Meth. 65: 55-63, 1983). An alternative assay format uses cells that are further engineered to express a reporter gene. The reporter gene is linked to a promoter element that is responsive to the receptor-linked pathway, and the assay detects activation of transcription of the reporter gene. Numerous reporter genes that are easily assayed for in cell extracts are known in the art, for example, the E. coli lacZ, chloroamphenicol acetyl transferase (CAT) and serum response element (SRE) (see, e.g., Shaw et al., Cell 56:563-72, 1989). An exemplary reporter gene is a luciferase gene (de Wet et al., Mol. Cell. Biol. 7:725, 1987). Expression of the luciferase gene is detected by luminescence using methods known in the art (e.g., Baumgartner et al., J. Biol. Chem. 269:29094-101, 1994; Schenborn and Goiffin, Promega Notes 41:11, 1993). Luciferase activity assay kits are commercially available from, for example, Promega Corp., Madison, Wis.

Provided immunomodulatory proteins can be characterized by the ability to inhibit the stimulation of human B cells by soluble APRIL or BAFF, as described by Gross et al, international publication No. WO00/40716. Briefly, human B cells are isolated from peripheral blood mononuclear cells, such as using CD19 magnetic beads separation (e.g. Miltenyi Biotec Auburn, CA). The purified B cells can be incubated under conditions of stimulation, e.g. in the presence of soluble APRIL, and further in the presence of titrated concentration of immunomodulatory protein. The B cells can be labeled with a proliferation dye or can be labeled with 1 μCi 3H-thymidine to measure proliferation. The number of B cells can be determined over time.

Reporter cell lines that express a reporter gene under the operable control of a transcription factor, such as NF-κB, NFAT-1 and AP-1, can be made that express TACI or BCMA. For example, the reporter cell can include Jurkat and other B Lymphoma cell lines. Incubation of these cells with soluble BAFF or APRIL ligands signal through the reporter genes in these constructs. The effect of provided immunomodulatory proteins to modulate this signaling can be assessed.

Well established animal models are available to test in vivo efficacy of provided immunomodulatory proteins in certain disease states, including those involving autoimmune or inflammatory conditions. For example, animal models of autoimmune disease include, for example, MRL-lpr/lpr or NZB×NZW F1 congenic mouse strains which serve as a model of SLE (systemic lupus erythematosus). Such animal models are known in the art, see for example Autoimmune Disease Models A Guidebook, Cohen and Miller eds. Academic Press. Offspring of a cross between New Zealand Black (NZB) and New Zealand White (NZW) mice develop a spontaneous form of SLE that closely resembles SLE in humans. The offspring mice, known as NZBW begin to develop IgM autoantibodies against T-cells at 1 month of age, and by 5-7 months of age, Ig anti-DNA autoantibodies are the dominant immunoglobulin. Polyclonal B-cell hyperactivity leads to overproduction of autoantibodies. The deposition of these autoantibodies, particularly ones directed against single stranded DNA is associated with the development of glomerulonephritis, which manifests clinically as proteinuria, azotemia, and death from renal failure. In some embodiments, proteinuria is determined by the urine total protein to creatinine ratio (UPCR; g/g). Kidney failure is the leading cause of death in mice affected with spontaneous SLE, and in the NZBW strain, this process is chronic and obliterative. The disease is more rapid and severe in females than males, with mean survival of only 245 days as compared to 406 days for the males. While many of the female mice will be symptomatic (proteinuria) by 7-9 months of age, some can be much younger or older when they develop symptoms. The fatal immune nephritis seen in the NZBW mice is very similar to the glomerulonephritis seen in human SLE, making this spontaneous murine model very attractive for testing of potential SLE therapeutics (Putterman and Naparstek, Murine Models of Spontaneous Systemic Lupus Erythematosus, Autoimmune Disease Models: A Guidebook, chapter 14, pp. 217-34, 1994; Mohan et al., J. Immunol. 154:1470-80, 1995; and Daikh et al., J. Immunol. 159:3104-08, 1997). Administration of provided immunomodulatory proteins to these mice to evaluate the efficacy to ameliorate symptoms and alterations to the course of disease can be assessed.

Another mouse model of inflammation and lupus-like disease is the bm12 inducible mouse model of SLE (Klarquist and Janssen, 2015. J. Vis. Exp. (105), e53319). Splenocyte suspensions from female I-A^{bm12}B6(C)-H2-Ab1^{bm12}/KhEgJ (‘bm12’) mice are adoptively transferred into female C57BL/6NJ recipient mice. H2-Ab1^{bm12} differs from H2-Ab1^{b} by 3 nucleotides, resulting in alteration of 3 amino acids in the f-chain of the MHC class 11 I-A molecule. Alloactivation of donor bm12 CD4+ T cells by recipient antigen presenting cells leads to chronic GVHD with symptoms closely resembling SLE, including autoantibody production, changes in immune cell subsets, and mild kidney disease. Glomerulonephritis with immune complex deposition develops late in the model, largely comprised of autoantigens bound to IgG1, IgG2b, IgG2c, and IgG3 antibodies. Endpoints of this model may include concentrations of anti-dsDNA antibodies, select IgG isotypes, blood urea nitrogen (BUN), and creatinine in serum, immune cell subset composition in the spleen and cervical LN, and kidney histology.

In some embodiments, mouse models for Sjögren's syndrome (SjS) can be used. The SjS disease, as well as an accelerated onset of diabetes, can be induced in female diabetes-prone non-obese diabetic (NOD) mice using repeat dosing with anti-mouse (m) PD-L1 antibody, based on a modified version of a protocol published by Zhou et al., 2016 Sci. Rep. 6, 39105. Starting at 6 weeks of age, mice are injected intraperitoneally (IP) on Study Days 0, 2, 4, and 6 with 100 μg of anti-PD-L1 antibody and are treated on various days with provided immunomodulatory proteins. Naïve mice are included as controls for the endpoint analyses. All mice are typically terminated on Study Day 10 and submandibular glands (SMG) and the pancreas from each mouse are collected for histopathology evaluation to assess for signs and severity of sialadenitis and insulitis. Blood glucose levels can be measured on various days.

In some embodiments, mouse models for experimental allergic encephalomyelitis (EAE) can be used. The models resemble human multiple sclerosis and produces demyelination as a result of T-cell activation to neuroproteins such as myelin basic protein (MBP), or proteolipid protein (PLP). Inoculation with antigen leads to induction of CD4+, class II MHC-restricted T-cells (Th1). Changes in the protocol for EAE can produce acute, chronic-relapsing, or passive-transfer variants of the model (Weinberg et al., J. Immunol. 162:1818-26, 1999; Mijaba et al., Cell. Immunol. 186:94-102, 1999; and Glabinski, Meth. Enzym. 288:182-90, 1997). Administration of provided immunomodulatory proteins to ameliorate symptoms and alterations to the course of disease can be assessed.

In some embodiments, a collagen-induced arthritis (CIA) model can be used in which mice develop chronic inflammatory arthritis which closely resembles human rheumatoid arthritis (RA). Since CIA shares similar immunological and pathological features with RA, this makes it an ideal model for screening potential human anti-inflammatory compounds. Another advantage in using the CIA model is that the mechanisms of pathogenesis are known. The T and B cell epitopes on type II collagen have been identified, and various immunological (delayed-type hypersensitivity and anti-collagen antibody) and inflammatory (cytokines, chemokines, and matrix-degrading enzymes) parameters relating to immune-mediating arthritis have been determined and can be used to assess test compound efficacy in the models (Wooley, Curr. Opin. Rheum. 3:407-20, 1999; Williams et al., Immunol. 89:9784-788, 1992; Myers et al., Life Sci. 61:1861-78, 1997; and Wang et al., Immunol. 92:8955-959, 1995). Administration of provided immunomodulatory proteins to ameliorate symptoms and alterations to the course of disease can be assessed.

In some embodiments, models for bronchial infection, such as asthma, can be created when mice are injected with ovalbumin and restimulated nasally with antigen which produces an asthmatic response in the bronchi similar to asthma. Administration of provided immunomodulatory proteins to ameliorate symptoms and alterations to the course of disease can be assessed.

In some embodiments, myasthenia gravis (MG) is another autoimmune disease for which murine models are available. MG is a disorder of neuromuscular transmission involving the production of autoantibodies directed against the nicotinic acetylcholine receptor (AChR). MG is acquired or inherited with clinical features including abnormal weakness and fatigue on exertion. A mouse model of MG has been established. (Christadoss et al., Establishment of a Mouse Model of Myasthenia Gravis Which Mimics Human Myasthenia Gravis Pathogenesis for Immune Intervention, in Immunobiology of Proteins and Peptides VIII, Atassi and Bixler, eds., 1995, pp. 195-99.) Experimental autoimmune myasthenia gravis (EAMG) is an antibody mediated disease characterized by the presence of antibodies to AChR. These antibodies destroy the receptor leading to defective neuromuscular electrical impulses, resulting in muscle weakness. In the EAMG model, mice are immunized with the nicotinic acetylcholine receptor. Clinical signs of MG become evident weeks after the second immunization. EAMG is evaluated by several methods including measuring serum levels of AChR antibodies by radioimmunoassay (Christadoss and Dauphinee, J. Immunol. 136:2437-40, 1986; and Lindstrom et al., Methods Enzymol. 74:432-60, 1981), measuring muscle AChR, or electromyography (Wu et al. Protocols in Immunology. Vol. 3, Eds. Coligen, Kruisbeak, Margulies, Shevach, and Strober. John Wiley and Sons, New York, p. 15.8.1, 1997).

Another use for in vivo models includes delivery of an antigen challenge to the animal followed by administration of immunomodulatory proteins and measuring the T and B cell response. T cell dependent and T cell independent immune response can be measured as described in Perez-Melgosa et al., J. Immunol. 163:1123-7, 1999. Immune response in animals subjected to a regular antigen challenge (for example, keyhole limpet hemacyanin (KLH), sheep red blood cells (SRBC), ovalbumin or collagen) followed by administration of provided immunomodulatory proteins can be done to measure effect on B cell response.

Pharmacokinetic studies can be used in association with radiolabeled immunomodulatory proteins to determine the distribution and half life of such polypeptides in vivo.

In some embodiments, modeling and simulation of pharmacokinetic (PK) and pharmacodynamic (PD) profiles observed in control animals and animal models of disease (e.g., cancer models) can be used to predict or determine patient dosing. For example, PK data from non-human primates (e.g., cynomolgus monkeys) can be used to estimate human PK. Similarly, mouse PK and PD data can be used to predict human dosing. The observed animal data can be used to inform computational models which can be used to simulate human dose response.

### VI. THERAPEUTIC APPLICATIONS

The pharmaceutical compositions described herein (including pharmaceutical composition comprising the immunomodulatory protein, e.g. TACI-Fc, described herein) can be used in a variety of therapeutic applications, such as in methods for the treatment of a disease. The therapeutic applications of the pharmaceutical compositions include methods and uses of any of the provided formulations. For example, in some embodiments the pharmaceutical composition, such as any provided formulation, is used to treat inflammatory or autoimmune disorders, cancer, organ transplantation, viral infections, and/or bacterial infections in a mammal. The pharmaceutical composition, such as any provided formulation, can modulate (e.g. decrease) an immune response to treat the disease.

Such methods and uses include therapeutic methods and uses, for example, involving administration of the molecules or compositions containing the same, to a subject having a disease, condition, or disorder. In some cases, such as described, the disease, condition or disorder is an autoimmune or inflammatory disease or disorder. In some embodiments, the molecule or engineered cell is administered in an effective amount to effect treatment of the disease or disorder. Uses include uses of molecules containing an immunomodulatory protein, and in the preparation of a medicament in order to carry out such therapeutic methods. In some embodiments, the methods are carried out by administering a provided immunomodulatory protein, or compositions comprising the same, to the subject having or suspected of having the disease or condition. In some embodiments, the methods thereby treat the disease, disorder or condition or disorder in the subject.

Illustrative subjects include mammalian subjects, such as farm animals, domestic animals, and human patients. In particular embodiments, the subject is a human subject.

The pharmaceutical compositions described herein can be used in a variety of therapeutic applications, such as the treatment of a disease. For example, in some embodiments the pharmaceutical composition is used to treat inflammatory or autoimmune disorders, organ transplantation, viral infections, and/or bacterial infections in a mammal. The pharmaceutical composition can modulate an immune response to treat the disease. In some embodiments, the pharmaceutical composition suppresses an immune response, which can be useful in the treatment of inflammatory or autoimmune disorders, or organ transplantation.

The provided methods are believed to have utility in a variety of applications, including, but not limited to, e.g., in prophylactic or therapeutic methods for treating a variety of immune system diseases or conditions in a mammal in which modulation or regulation of the immune system and immune system responses is beneficial. For example, suppressing an immune response can be beneficial in prophylactic and/or therapeutic methods for inhibiting rejection of a tissue, cell, or organ transplant from a donor by a recipient. In a therapeutic context, the mammalian subject is typically one with an immune system disease or condition, and administration is conducted to prevent further progression of the disease or condition.

### A. Diseases or Disorders for Treatment

The provided immunomodulatory proteins, including TACI fusion proteins, can be used for the treatment of autoimmune diseases, B cell cancers, immunomodulation, EBD and any antibody-mediated pathologies (e.g., ITCP, myasthenia gravis and the like), renal diseases, indirect T cell immune response, graft rejection, and graft versus host disease. Administration of the immunomodulatory proteins (e.g. TACI-Fc) can specifically regulate B cell responses during the immune response. Additionally, administration of provided immunomodulatory proteins can be used to modulate B cell development, development of other cells, antibody production, and cytokine production. Administration or use of provided immunomodulatory proteins can also modulate B cell communication, such as by neutralizing the proliferative effects of BAFF or APRIL.

In some embodiments, the pharmaceutical composition suppresses an immune response, which can be useful in the treatment of inflammatory or autoimmune disorders, or organ transplantation. In some embodiments, the pharmaceutical composition contains an immunomodulatory protein that exhibits antagonist activity of a B cell stimulatory receptor, thereby decreasing or reducing an immune response. In some embodiments, the pharmaceutical compositions can be used to treat a B cell-mediated disease.

In some embodiments, the pharmaceutical compositions can be used to treat an autoimmune disease. In some embodiments, the administration of a pharmaceutical composition containing an immunomodulatory protein provided herein to a subject suffering from an immune system disease (e.g., autoimmune disease) can result in suppression or inhibition of such immune system attack or biological responses associated therewith. By suppressing this immune system attack on healthy body tissues, the resulting physical symptoms (e.g., pain, joint inflammation, joint swelling or tenderness) resulting from or associated with such attack on healthy tissues can be decreased or alleviated, and the biological and physical damage resulting from or associated with the immune system attack can be decreased, retarded, or stopped. In a prophylactic context, the subject may be one with, susceptible to, or believed to present an immune system disease, disorder or condition, and administration is typically conducted to prevent progression of the disease, disorder or condition, inhibit or alleviate symptoms, signs, or biological responses associated therewith, prevent bodily damage potentially resulting therefrom, and/or maintain or improve the subject's physical functioning.

In some embodiments, the disease or conditions that can be treated by the pharmaceutical composition described herein is any disease mediated by immune complex deposition (e.g. lupus nephritis, vasculitis); direct interference with a pathway (e.g. catastrophic antiphospholipid antibody syndrome, myasthenia gravis crisis; anti-Jo-1 disease); opsonization or direct damage to cells (e.g. Idiopathic thrombocytopenic purpura, autoimmune hemolytic anemia); antibody-mediated rejection of an allograft (e.g. highly-sensitized renal transplant patients); or anti-drug antibodies to biologic replacement factors, vectors (e.g. anti-Factor 8).

In some embodiments, the inflammatory or autoimmune disorders, conditions or diseases that can be treated by the pharmaceutical composition described herein is Systemic lupus erythematosus (SLE), including flare prevention without glucocorticoids; Sjogren's syndrome; Primary biliary cirrhosis (PBC); Systemic scleroderma; Polymyositis; Diabetes prevention; IgA nephropathy; IgA vasculitis; B cell cancers, for example myeloma; Multiple sclerosis, Optic neuritis.

In some embodiments, the inflammatory or autoimmune disorder is an inflammatory arthritis. Examples of inflammatory arthritis for treatment in accord with the provided methods include, but are not limited to rheumatoid arthritis, psoriatic arthritis, lupus, lyme disease, gout, or ankylosing spondylitis.

In some embodiments, the provided pharmaceutical compositions can be used to treat pre-B or B-cell leukemias, such as plasma cell leukemia, chronic or acute lymphocytic leukemia, myelomas such as multiple myeloma, plasma cell myeloma, endothelial myeloma and giant cell myeloma, and lymphomas such as non-Hodgkins lymphoma. In some of any embodiments, the type of myeloma includes multiple myeloma, plasmacytoma, multiple solitary plasmacytoma, and/or extramedullary myeloma. In some of any embodiments, the type of myeloma includes light chain myeloma, nonsecretory myeloma, and/or IgD or IgE myeloma.

In some embodiments, the provided pharmaceutical compositions can be used as immunosuppressants to selectively block the action of B-lymphocytes for use in treating disease. For example, certain autoimmune diseases are characterized by production of autoantibodies, which contribute to tissue destruction and exacerbation of disease. Autoantibodies can also lead to the occurrence of immune complex deposition complications and lead to many symptoms of systemic lupus erythematosus, including kidney failure, neuralgic symptoms and death. Modulating antibody production independent of cellular response would also be beneficial in many disease states. B cells have also been shown to play a role in the secretion of arthritogenic immunoglobulins in rheumatoid arthritis. Methods and uses of the provided immunomodulatory proteins to inhibit, block or neutralize action of B cells to thereby suppress antibody production would be beneficial in treatment of autoimmune diseases such as myasthenia gravis, rheumatoid arthritis, polyarticular-course juvenile rheumatoid arthritis, and psoriatic arthritis.

In some embodiments, the provided pharmaceutical compositions can be used to block or neutralize the actions of B-cells in association with end stage renal diseases, which may or may not be associated with autoimmune diseases. Such methods would also be useful for treating immunologic renal diseases. Such methods would be useful for treating glomerulonephritis associated with diseases such as membranous nephropathy, IgA nephropathy or Berger's Disease, IgM nephropathy, IgA Vasculitis, Goodpasture's Disease, post-infectious glomerulonephritis, mesangioproliferative disease, chronic lymphoid leukemia, minimal-change nephrotic syndrome. Such methods would also serve as therapeutic applications for treating secondary glomerulonephritis or vasculitis associated with such diseases as lupus, polyarteritis, Henoch-Schonlein, Scleroderma, HTV-related diseases, amyloidosis or hemolytic uremic syndrome. The provided methods would also be useful as part of a therapeutic application for treating interstitial nephritis or pyelonephritis associated with chronic pyelonephritis, analgesic abuse, nephrocalcinosis, nephropathy caused by other agents, nephrolithiasis, or chronic or acute interstitial nephritis. The methods provided herein also include use of the provided immunomodulatory proteins in the treatment of hypertensive or large vessel diseases, including renal artery stenosis or occlusion and cholesterol emboli or renal emboli. The provided methods and uses also can be used for treatment of renal or urological neoplasms, multiple myelomas, lymphomas, light chain neuropathy or amyloidosis.

In some embodiments, the provided pharmaceutical compositions also can be used for the treatment of asthma and other chronic airway diseases such as bronchitis and emphysema. The provided immunomodulatory proteins can also be used to treat Sjogren's Syndrome.

In some embodiments, methods and uses of the provided pharmaceutical compositions include immunosuppression, in particular for such therapeutic use as for graft-versus-host disease and graft rejection. In some embodiments, methods and uses of the provided immunomodulatory proteins include treatment of such autoimmune diseases as insulin dependent diabetes mellitus (IDDM) and Crohn's Disease. Methods provided herein would have additional therapeutic value for treating chronic inflammatory diseases, in particular to lessen joint pain, swelling, anemia and other associated symptoms as well as treating septic shock.

In some embodiments, the pharmaceutical compositions can be used to treat an autoantibody-related disease or disorder.

In some embodiments, the autoantibody-related disease or disorder treated by a pharmaceutical composition containing an immunomodulatory protein described herein includes, but is not limited to, a rheumatic disease or disorder, a hematologic disease or disorder, a dermatologic disease or disorder, or a neurologic disease or disorder. In some embodiments, the autoantibody-related disease or disorder comprises cytopenia, Pemphigus foliaceus, blistering disease or encephalitis. In some embodiments, the encephalitis comprises limbic encephalitis.

In some embodiments, the inflammatory and autoimmune disorders that can be treated by a pharmaceutical composition containing an immunomodulatory protein described herein include, but are not limited to, Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune adrenalitis (Addison's disease); Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune polyglandular syndrome type II (APS II); Autoimmune retinopathy; Autoimmune thyroid disease (AITD), i.e. Hashimoto's disease; Autoimmune urticarial; Axonal & neuronal neuropathy (AMAN); Baló disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosum; Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inversa); Hypogammalglobulinemia; IgA Nephropathy; IgA Vasculitis; IgG4-related sclerosing disease; Immune thrombocytopenic purpura (ITP); Inclusion body myositis (IBM); Interstitial cystitis (IC); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus; Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB; Multiple sclerosis; Myasthenia gravis; Myositis; Narcolepsy; Neonatal Lupus; Neuromyelitis optica (NMO); Neuromyelitis optica spectrum disorder (NMOSD); Neutropenia; Ocular cicatricial pemphigoid; Optic neuritis; Palindromic rheumatism (PR); PANDAS; Paraneoplastic cerebellar degeneration (PCD); Paroxysmal nocturnal hemoglobinuria (PNH); Parry Romberg syndrome; Pars planitis (peripheral uveitis); Parsonage-Turner syndrome; Pemphigus, Pemphigus vulgaris; Peripheral neuropathy; Perivenous encephalomyelitis; Pernicious anemia (PA); POEMS syndrome; Polyarteritis nodosa; Polyglandular syndromes type I, II, III; Polymyalgia rheumatic; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PRCA); Pyoderma gangrenosum; Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); systemic lupus erythematosus (SLE); Takayasu's arteritis; Temporal arteritis/Giant cell arteritis; Thrombocytopenic purpura (TTP); Tolosa-Hunt syndrome (THS); Transverse myelitis; Type 1 diabetes; Ulcerative colitis (UC); Undifferentiated connective tissue disease (UCTD); Uveitis; Vasculitis; Vitiligo or Vogt-Koyanagi-Harada Disease. In some embodiments, the provided immunomodulatory proteins (e.g. TACI-Fc) can be used to treat Scleroderma, Myasthenia gravis, GVHD (including acute GVHD or chronic GVHD), an immune response in connection with transplantation; Antiphospholipid Ab syndrome; Multiple sclerosis; Sjogren's syndrome; IgG4-related disease; Type I diabetes; Rheumatoid arthritis including glucocorticoid therapy (GC) RA or Acute lupus nephritis.

In some embodiments, the provided pharmaceutical compositions can be used to treat Amyotrophic lateral sclerosis, Neuromyelitis optica (NMO), Neuromyelitis optica spectrum disorder (NMOSD), Transverse myelitis, CNS autoimmunity, Guillain-barre syndrome, Neurocystercercosis, Sarcoidosis (T/seroneg), Churg-Strauss Syndrome, Hashimoto's thyroiditis, Grave's disease, immune thrombocytopenia (ITP), Addison's Disease, Polymyositis, or Dermatomyositis.

In some embodiments, the provided pharmaceutical compositions can be used to treat IgA nephropathy, chronic inflammatory demyelinating polyneuropathy (CIDP), antisynthetase disease such as Jo-1 syndrome, or ANCA vasculitis.

In some embodiments, the provided pharmaceutical compositions can be used to treat an autoantibody-associated glomerular disease. In some embodiments, the autoantibody-associated glomerular disease may include immunoglobulin (Ig) A nephropathy (IgAN), lupus nephritis (LN), primary membranous nephropathy (pMN), or renal anti-neutrophil cytoplasmic antibody (ANCA)-associated vasculitis (AAV).

In some embodiments, the provided immunomodulatory proteins (e.g. TACI-Fc) can be used to treat systemic lupus erythematosus (SLE).

In some embodiments, the provided immunomodulatory proteins (e.g. TACI-Fc) can be used to treat Sjogren's syndrome (SjS).

In some embodiments, the provided immunomodulatory proteins (e.g. TACI-Fc) can be used to treat autoimmune myasthenia gravis.

In some embodiments, the provided pharmaceutical compositions can be used to treat a B cell cancer. In some embodiments, the B cell cancer is a cancer in which BAFF and APRIL are involved or implicated in providing an autocrine survival loop to the B cells. In some embodiments, the cancer is B cell chronic lymphocytic leukemia, non-Hodgkins' lymphoma or myeloma. In some embodiments, the cancer is myeloma. In some of any embodiments, the type of myeloma includes multiple myeloma, plasmacytoma, multiple solitary plasmacytoma, and/or extramedullary myeloma. In some of any embodiments, the type of myeloma includes light chain myeloma, nonsecretory myeloma, and/or IgD or IgE myeloma.

In some embodiments, the subject may receive standard of care (SOC) therapy for their underlying disorder, which is within the level of skill of the investigator or clinical physician. In some embodiments, the subject has previously received the SOC therapy prior to receiving the provided TACI-Fc fusion protein. In some embodiments, the subject continues receiving the SOC therapy while receiving administration of the provided TACI-Fc fusion protein. In some embodiments, the SOC therapy is tapered over time after receiving administration of the provided TACI-Fc fusion protein.

In some embodiments, the subject has not received an agent that directly depletes B lymphocytes (e.g. Rituximab) within 48 weeks prior to initiation of administration of the provided immunomodulatory proteins (e.g. TACI-Fc). In some embodiments, the subject may have received an agent that directly depletes B lymphocytes (e.g. Rituximab) within greater than 24 weeks prior to initiation of administration of the provided immunomodulatory proteins (e.g. TACI-Fc) if B cells have returned to normal reference ranges prior to administration of the TACI-Fc fusion protein.

In some embodiments, the subject has not received an agent that directly inhibits BAFF and/or APRIL, such as Belimumab, within 24 weeks prior to initiation of administration of the provided immunomodulatory proteins (e.g. TACI-Fc).

In some embodiments, the subject has not received administration of Intravenous Ig, abatacept, anifrolumab, belatacept, adalimumab, infliximab, certolizumab, etanercept, golimumab, anakinra, canakinumab, tocilizumab, sarilumab, satralizumab within 8 weeks prior to initiation of administration of the provided immunomodulatory proteins (e.g. TACI-Fc). In some embodiments, the subject has not received administration of any approved therapeutic agent for treating an immune disease within 8 weeks prior to initiation of administration of the provided immunomodulatory proteins (e.g. TACI-Fc).

In some embodiments, the subject has not received cyclophosphamide within 8 weeks prior to initiation of administration of the provided immunomodulatory proteins (e.g. TACI-Fc).

### B. Dosing and Administration

In some embodiments, a therapeutic amount of the pharmaceutical composition is administered. Typically, a precise amount of the pharmaceutical compositions of the present invention to be administered can be determined by a physician with consideration of individual differences in age, weight, extent of infection, and condition of the patient (subject). The optimal dosage and treatment regimen for a particular patient can readily be determined by one skilled in the art of medicine by monitoring the patient for signs of disease and adjusting the treatment accordingly.

In some embodiments, the subject is human. In some embodiments, the subject is an adult subject. In some embodiments, the subject is greater than or equal to 18 years of age.

In some embodiments, a pharmaceutical composition described herein (including a pharmaceutical composition comprising any of the TACI-Fc fusion proteins described herein) is administered to a subject. Generally, dosages and routes of administration of the pharmaceutical composition are determined according to the size and condition of the subject, according to standard pharmaceutical practice. For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage can be determined in light of factors related to the subject requiring treatment. Dosage and administration can be adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy.

In some embodiments, modeling and simulation of pharmacokinetic (PK) and pharmacodynamic (PD) profiles observed in control animals and animal models of disease (e.g., cancer models) can be used to predict or determine patient dosing. For example, PK data from non-human primates (e.g., cynomolgus monkeys) can be used to estimate human PK. Similarly, mouse or rat PK and PD data can be used to predict human dosing. The observed animal data can be used to inform computational models which can be used to simulate human dose response.

In some embodiments, methods provided herein include administering a pharmaceutical composition described herein (including pharmaceutical composition comprising a TACI-Fc fusion proteins) in an amount in which a dose is known or predicted to neutralize an activity of APRIL or BAFF ligand, including a BAFF or APRIL homotrimer, a BAFF/APRIL heterotimer or a BAFF 60mer, sufficient for a therapeutic effect. The particular amount can be determined experimentally or empirically. In some embodiments, the amount can be empirically determined from in vitro binding data or from animal models.

In some embodiments, the TACI-Fc fusion protein, or pharmaceutical compositions thereof, may be administered every 3 to 4 days, once every week, biweekly, every three weeks, once a month, once every two months, or once every three months. The precise timing and frequency can be empirically determined by a skilled clinician or physician, such as depending on the particular half-life and clearance rate of the particular formulation. The frequency of dosing will depend upon the pharmacokinetic parameters of the molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

In some cases, for example when a chronic inflammatory or autoimmune disorder is treated with a pharmaceutical composition provided herein, such as a TACI-Fc fusion protein provided herein, the composition is administered continuously, e.g., repeatedly, over time or intermittently over time. The duration of administration can be for weeks, months or years. In some cases, treatment of a chronic inflammatory or autoimmune disorder, e.g., with a pharmaceutical composition provided herein, such as a containing a TACI-Fc fusion protein provided herein, may include administering the treatment to a subject indefinitely. In some embodiments, when the inflammatory or autoimmune disorder is a chronic inflammatory or autoimmune disorder, treatment with a pharmaceutical composition provided herein, such as a TACI-Fc fusion protein provided herein, is continued following remission or partial remission of the disease and/or a reduction or amelioration in signs and/or symptoms of a disease, such as a reduction of one or more signs of inflammation in a subject having the chronic inflammatory or autoimmune disorder. In some embodiments, administration continues until any time as desired by a skilled practitioner. In some cases, for example when an acute inflammatory or autoimmune disorder is treated with a pharmaceutical composition provided herein, such as a TACI-Fc fusion protein provided herein, the composition is administered for a defined or limited period of time. In some embodiments, when the inflammatory or autoimmune disorder is an acute inflammatory or autoimmune disorder, treatment with a pharmaceutical composition provided herein, such as a TACI-Fc fusion protein provided herein, is discontinued following remission or partial remission of the disease and/or a reduction or amelioration in signs and/or symptoms of a disease, such as a reduction of one or more signs of inflammation in a subject having the acute inflammatory or autoimmune disorder. In some embodiments, administration is discontinued at any time as desired by a skilled practitioner.

Typically, precise amount of the compositions of the present invention to be administered can be determined by a physician with consideration of individual differences in age, weight, tumor size, extent of infection or metastasis, and condition of the patient (subject). In some embodiments, when referencing dosage based on mg/kg of the subject, an average human subject is considered to have a mass of about 70 kg-75 kg, such as 70 kg and a body surface area (BSA) of 1.73 m2.

In some embodiments, the dosage of the pharmaceutical composition is a single dose or a repeated dose. In some embodiments, the doses are given to a subject once per day, twice per day, three times per day, or four or more times per day. In some embodiments, about 1 or more (such as about 2 or more, about 3 or more, about 4 or more, about 5 or more, about 6 or more, or about 7 or more) doses are given in a week. In some embodiments, multiple doses are given over the course of days, weeks, months, or years. In some embodiments, a course of treatment is about 1 or more doses (such as about 2 or more does, about 3 or more doses, about 4 or more doses, about 5 or more doses, about 7 or more doses, about 10 or more doses, about 15 or more doses, about 25 or more doses, about 40 or more doses, about 50 or more doses, or about 100 or more doses).

In particular embodiments, a TACI-Fc fusion protein is administered as a plurality of doses where each dose is administered no more than once weekly. In some embodiments, each does is administered once a week (Q1W). In some embodiments, each dose is administered once every two weeks (Q2W). In some embodiments, each dose is administered once every three weeks (Q3W). In some embodiments, each dose is administered once every four weeks (Q4W). In some embodiments, each dose is administered once every two months (e.g. Q8W). In some embodiments, each dose is administered once every three months (e.g. Q12W). In some embodiments, each dose is administered once every 6 months (e.g., Q24W). In aspects of provided embodiments, the administration cycle is repeated a plurality of times to administer a plurality of doses of the TACI-Fc fusion protein. In some embodiments, the administration is continued for a predetermined period of time, e.g. 4 weeks, 6 weeks, 8 weeks, 3 months, 6 months, 1 year or more. In some embodiments, the administration is discontinued after relapse or progression of the disease or condition in the subject.

In some embodiments, a dose regimen as described herein is administered to achieve a therapeutically effective amount to treat the disease, disorder or condition in the subject in need thereof. In some embodiments, each dose of the TACI-Fc fusion protein is administered in an amount between at or about 2.4 mg and at or about 960 mg, inclusive. In some embodiments, each dose of the TACI-Fc fusion protein is administered in an amount between at or about 8 mg and at or about 960 mg, between at or about 8 mg and at or about 880 mg, between at or about 8 mg and at or about 800 mg, between at or about 8 mg and at or about 720 mg, between at or about 8 mg and at or about 640 mg, between at or about 8 mg and at or about 560 mg, between at or about 8 mg and at or about 480 mg, between at or about 8 mg and at or about 400 mg, between at or about 8 mg and at or about 320 mg, between at or about 8 mg and at or about 240 mg, between at or about 8 mg and at or about 160 mg, between at or about 8 mg and at or about 80 mg, between at or about 8 mg and at or about 40 mg, between at or about 40 mg and at or about 960 mg, between at or about 40 mg and at or about 880 mg, between at or about 40 mg and at or about 800 mg, between at or about 40 mg and at or about 720 mg, between at or about 40 mg and at or about 640 mg, between at or about 40 mg and at or about 560 mg, between at or about 40 mg and at or about 480 mg, between at or about 40 mg and at or about 400 mg, between at or about 40 mg and at or about 320 mg, between at or about 40 mg and at or about 240 mg, between at or about 40 mg and at or about 160 mg, between at or about 40 mg and at or about 80 mg, between at or about 80 mg and at or about 960 mg, between at or about 80 mg and at or about 880 mg, between at or about 80 mg and at or about 800 mg, between at or about 80 mg and at or about 720 mg, between at or about 80 mg and at or about 640 mg, between at or about 80 mg and at or about 560 mg, between at or about 80 mg and at or about 480 mg, between at or about 80 mg and at or about 400 mg, between at or about 80 mg and at or about 320 mg, between at or about 80 mg and at or about 240 mg, between at or about 80 mg and at or about 160 mg, between at or about 160 mg and at or about 960 mg, between at or about 160 mg and at or about 880 mg, between at or about 160 mg and at or about 800 mg, between at or about 160 mg and at or about 720 mg, between at or about 160 mg and at or about 640 mg, between at or about 160 mg and at or about 560 mg, between at or about 160 mg and at or about 480 mg, between at or about 160 mg and at or about 400 mg, between at or about 160 mg and at or about 320 mg, between at or about 160 mg and at or about 240 mg, between at or about 240 mg and at or about 960 mg, between at or about 240 mg and at or about 880 mg, between at or about 240 mg and at or about 800 mg, between at or about 240 mg and at or about 720 mg, between at or about 240 mg and at or about 640 mg, between at or about 240 mg and at or about 560 mg, between at or about 240 mg and at or about 480 mg, between at or about 240 mg and at or about 400 mg, between at or about 240 mg and at or about 320 mg, between at or about 320 mg and at or about 960 mg, between at or about 320 mg and at or about 880 mg, between at or about 320 mg and at or about 800 mg, between at or about 320 mg and at or about 720 mg, between at or about 320 mg and at or about 640 mg, between at or about 320 mg and at or about 560 mg, between at or about 320 mg and at or about 480 mg, between at or about 320 mg and at or about 400 mg, between at or about 400 mg and at or about 960 mg, between at or about 400 mg and at or about 880 mg, between at or about 400 mg and at or about 800 mg, between at or about 400 mg and at or about 720 mg, between at or about 400 mg and at or about 640 mg, between at or about 400 mg and at or about 560 mg, between at or about 400 mg and at or about 480 mg, between at or about 480 mg and at or about 960 mg, between at or about 480 mg and at or about 880 mg, between at or about 480 mg and at or about 800 mg, between at or about 480 mg and at or about 720 mg, between at or about 480 mg and at or about 640 mg, between at or about 480 mg and at or about 560 mg, between at or about 560 mg and at or about 960 mg, between at or about 560 mg and at or about 880 mg, between at or about 560 mg and at or about 800 mg, between at or about 560 mg and at or about 720 mg, between at or about 560 mg and at or about 640 mg, between at or about 640 mg and at or about 960 mg, between at or about 640 mg and at or about 880 mg, between at or about 640 mg and at or about 800 mg, between at or about 640 mg and at or about 720 mg, between at or about 720 mg and at or about 960 mg, between at or about 720 mg and at or about 880 mg, between at or about 720 mg and at or about 800 mg, between at or about 800 mg at or about 960 mg, between at or about 800 mg and at or about 880 mg or between at or about 880 mg and at or about 960 mg, each inclusive.

In some embodiments, each dose of the TACI-Fc fusion protein is administered in an amount between at or about 8 mg and at or about 20 mg, between at or about 20 mg and at or about 960 mg, between at or about 20 mg and at or about 880 mg, between at or about 20 mg and at or about 800 mg, between at or about 20 mg and at or about 720 mg, between at or about 20 mg and at or about 640 mg, between at or about 20 mg and at or about 560 mg, between at or about 20 mg and at or about 480 mg, between at or about 20 mg and at or about 400 mg, between at or about 20 mg and at or about 320 mg, between at or about 20 mg and at or about 240 mg, between at or about 20 mg and at or about 160 mg, between at or about 20 mg and at or about 40 mg, each inclusive.

In some embodiments, each dose of the TACI-Fc fusion protein is administered in an amount between at or about 8 mg and at or about 20 mg, between at or about 20 mg and at or about 960 mg, between at or about 20 mg and at or about 880 mg, between at or about 20 mg and at or about 800 mg, between at or about 20 mg and at or about 720 mg, between at or about 20 mg and at or about 640 mg, between at or about 20 mg and at or about 560 mg, between at or about 20 mg and at or about 480 mg, between at or about 20 mg and at or about 400 mg, between at or about 20 mg and at or about 320 mg, between at or about 20 mg and at or about 240 mg, between at or about 20 mg and at or about 160 mg, between at or about 20 mg and at or about 40 mg, each inclusive.

In some embodiments, each dose of a TACI-Fc fusion protein is or is about 2.4 mg. In some embodiments, each dose of a TACI-Fc fusion protein is or is about 8 mg. In some embodiments, each dose of a TACI-Fc fusion protein is or is about 20 mg. In some embodiments, each dose of a TACI-Fc fusion protein is or is about 24 mg. In some embodiments, each dose of a TACI-Fc fusion protein is or is about 40 mg. In some embodiments, each dose of a TACI-Fc fusion protein is or is about 80 mg. In some embodiments, each dose of a TACI-Fc fusion protein is or is about 160 mg. In some embodiments, each dose of a TACI-Fc fusion protein is or is about 240 mg. In some embodiments, each dose of a TACI-Fc fusion protein is or is about 320 mg. In some embodiments, each dose of a TACI-Fc fusion protein is or is about 400 mg. In some embodiments, each dose of a TACI-Fc fusion protein is or is about 480 mg. In some embodiments, each dose of a TACI-Fc fusion protein is or is about 560 mg. In some embodiments, each dose of a TACI-Fc fusion protein is or is about 640 mg. In some embodiments, each dose of a TACI-Fc fusion protein is or is about 720 mg. In some embodiments, each dose of a TACI-Fc fusion protein is or is about 800 mg. In some embodiments, each dose of a TACI-Fc fusion protein is or is about 880 mg. In some embodiments, each dose of a TACI-Fc fusion protein is or is about 960 mg.

In some embodiments, the dose is an amount between or between about 40 mg and at or about 480 mg, between at or about 80 mg to at or about 320 mg, or between at or at or about 80 mg to at or about 120 mg, each inclusive.

In some embodiments, each dose of the TACI-Fc fusion protein is administered once every three months. In some embodiments, the TACI-Fc fusion protein is administered in an amount from at or about 160 mg to at or about 960 mg once every three months. In some embodiments, the TACI-Fc fusion protein is administered in an amount from at or about 240 mg to at or about 800 mg once every three months. In some embodiments, the TACI-Fc fusion protein is administered in an amount from at or about 480 mg to at or about 720 mg once every three months.

In some embodiments, each dose of the TACI-Fc fusion protein is administered once every month (once every four weeks or Q4W). In some embodiments, the TACI-Fc fusion protein is administered in an amount from at or about 2.4 mg to at or about 960 mg Q4W. In some embodiments, the TACI-Fc fusion protein is administered in an amount from at or about 80 mg to at or about 720 mg Q4W. In some embodiments, the TACI-Fc fusion protein is administered in an amount from at or about 160 mg to at or about 560 mg Q4W. In some embodiments, the TACI-Fc fusion protein is administered in an amount from at or about 240 mg to at or about 480 mg Q4W. In some embodiments, the TACI-Fc fusion protein is administered at or about 24 mg Q4W. In some embodiments, the TACI-Fc fusion protein is administered at or about 80 mg Q4W. In some embodiments, the TACI-Fc fusion protein is administered at or about 160 mg Q4W. In some embodiments, the TACI-Fc fusion protein is administered at or about 240 mg Q4W. In some embodiments, the TACI-Fc fusion protein is administered subcutaneously. In some embodiments, the TACI-Fc fusion protein is administered intravenously.

In some embodiments, each dose of the TACI-Fc fusion protein is administered once every other week (Q2W). In some embodiments, the TACI-Fc fusion protein is administered in an amount from at or about 2.4 mg to at or about 960 mg Q2W. In some embodiments, the TACI-Fc fusion protein is administered in an amount from at or about 80 mg to at or about 720 mg Q2W. In some embodiments, the TACI-Fc fusion protein is administered in an amount from at or about 160 mg to at or about 560 mg Q2W. In some embodiments, the TACI-Fc fusion protein is administered in an amount from at or about 240 mg to at or about 480 mg Q2W. In some embodiments, the TACI-Fc fusion protein is administered at or about 80 mg Q2W. In some embodiments, the TACI-Fc fusion protein is administered at or about 160 mg Q2W. In some embodiments, the TACI-Fc fusion protein is administered at or about 240 mg Q2W. In some embodiments, the TACI-Fc fusion protein is administered subcutaneously. In some embodiments, the TACI-Fc fusion protein is administered intravenously.

In some embodiments, each dose of the TACI-Fc fusion protein is administered once every two months (Q8W). In some embodiments, the TACI-Fc fusion protein is administered in an amount from at or about 2.4 mg to at or about 960 mg Q8W. In some embodiments, the TACI-Fc fusion protein is administered in an amount from at or about 80 mg to at or about 720 mg Q8W. In some embodiments, the TACI-Fc fusion protein is administered in an amount from at or about 160 mg to at or about 560 mg Q8W. In some embodiments, the TACI-Fc fusion protein is administered in an amount from at or about 240 mg to at or about 480 mg Q8W. In some embodiments, the TACI-Fc fusion protein is administered at or about 24 mg Q8W. In some embodiments, the TACI-Fc fusion protein is administered at or about 80 mg Q8W. In some embodiments, the TACI-Fc fusion protein is administered at or about 160 mg Q8W. In some embodiments, the TACI-Fc fusion protein is administered at or about 240 mg Q8W. In some embodiments, the TACI-Fc fusion protein is administered at or about 320 mg Q8W. In some embodiments, the TACI-Fc fusion protein is administered at or about 480 mg Q8W. In some embodiments, the TACI-Fc fusion protein is administered subcutaneously. In some embodiments, the TACI-Fc fusion protein is administered intravenously.

In some embodiments, each dose of the TACI-Fc fusion protein is administered once every three months (Q12W). In some embodiments, the TACI-Fc fusion protein is administered in an amount from at or about 2.4 mg to at or about 960 mg Q12W. In some embodiments, the TACI-Fc fusion protein is administered in an amount from at or about 80 mg to at or about 720 mg Q12W. In some embodiments, the TACI-Fc fusion protein is administered in an amount from at or about 160 mg to at or about 560 mg Q12W. In some embodiments, the TACI-Fc fusion protein is administered in an amount from at or about 240 mg to at or about 480 mg Q12W. In some embodiments, the TACI-Fc fusion protein is administered at or about 24 mg Q12W. In some embodiments, the TACI-Fc fusion protein is administered at or about 80 mg Q12W. In some embodiments, the TACI-Fc fusion protein is administered at or about 160 mg Q12W. In some embodiments, the TACI-Fc fusion protein is administered at or about 240 mg Q12W. In some embodiments, the TACI-Fc fusion protein is administered subcutaneously. In some embodiments, the TACI-Fc fusion protein is administered intravenously.

In some embodiments, each dose of the TACI-Fc fusion protein is administered once a week (Q1W). In some embodiments, the TACI-Fc fusion protein is administered in an amount from at or about 2.4 mg to at or about 960 mg Q1W. In some embodiments, the TACI-Fc fusion protein is administered in an amount from at or about 40 mg to at or about 480 mg Q1W. In some embodiments, the TACI-Fc fusion protein is administered in an amount from at or about 80 mg to at or about 320 mg Q1W. In some embodiments, the TACI-Fc fusion protein is administered in an amount from at or about 80 mg and at or about 120 mg Q1W.

In some embodiments, each dose of the TACI-Fc fusion protein is administered once every six months (Q24W). In some embodiments, the TACI-Fc fusion protein is administered in an amount from at or about 2.4 mg to at or about 960 mg Q24W. In some embodiments, the TACI-Fc fusion protein is administered in an amount from at or about 80 mg to at or about 720 mg Q24W. In some embodiments, the TACI-Fc fusion protein is administered in an amount from at or about 160 mg to at or about 560 mg Q24W. In some embodiments, the TACI-Fc fusion protein is administered in an amount from at or about 240 mg to at or about 480 mg Q24W. In some embodiments, the TACI-Fc fusion protein is administered at or about 24 mg Q24W. In some embodiments, the TACI-Fc fusion protein is administered at or about 80 mg Q24W. In some embodiments, the TACI-Fc fusion protein is administered at or about 160 mg Q24W. In some embodiments, the TACI-Fc fusion protein is administered at or about 240 mg Q24W. In some embodiments, the TACI-Fc fusion protein is administered subcutaneously. In some embodiments, the TACI-Fc fusion protein is administered intravenously.

The present disclosure is based on the surprising results that the TACI-Fc fusion protein provided herein is well-tolerated (i.e., no major adverse effects) and a low dose is effective at inhibiting BAFF and APRIL, and related immune responses, compared to published BAFF and/or APRIL inhibitors (e.g., Atacicept, Telitacicept, BION-1301 and Sibeprenlimab). Thus, in some embodiments, the TACI-Fc fusion protein is administered subcutaneously in an amount from at or about 80 mg to at or about 480 mg Q4W. In some embodiments, the TACI-Fc fusion protein is administered subcutaneously in an amount at or about 80 mg Q4W. In some embodiments, the TACI-Fc fusion protein is administered subcutaneously in an amount at or about 160 mg Q4W. In some embodiments, the TACI-Fc fusion protein is administered subcutaneously in an amount at or about 240 mg Q4W. In some embodiments, the TACI-Fc fusion protein is administered subcutaneously in an amount at or about 480 mg Q4W.

It is contemplated that dosing (e.g., multiple doses), can continue until any time as desired by a skilled practitioner. For example, dosing may continue until a desirable disease response is achieved, such as in remission or partial remission of the disease and/or a reduction or amelioration in signs and/or symptoms of a disease, such as a reduction of one or more signs of inflammation in the subject. In some embodiments, the dosing is continued following remission or partial remission of the disease and/or a reduction or amelioration in signs and/or symptoms of a disease, such as a reduction of one or more signs of inflammation in the subject.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the therapeutic composition is administered to a patient by intradermal or subcutaneous injection. In another embodiment, the therapeutic composition is administered by i.v. injection.

In some embodiments, the pharmaceutical composition (including pharmaceutical compositions comprising any of the TACI-Fc fusion proteins described herein) is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, intraspinally, intratumorally or systemically.

In some embodiments, the pharmaceutical composition (including pharmaceutical compositions comprising any of the TACI-Fc fusion proteins described herein) is administered to a subject via subcutaneous administrations. In some embodiments, the dose of the TACI-Fc fusion for subcutaneous administration is at or about 80 mg. In some embodiments, the dose of the TACI-Fc fusion for subcutaneous administration is at or about 240 mg. In some embodiments, the dose of the TACI-Fc fusion for subcutaneous administration is at or about 480 mg. In some embodiments, the dose of the TACI-Fc fusion for subcutaneous administration is at or about 720 mg. In some embodiments, each dose is administered subcutaneously Q1W. In some embodiments, each dose is administered subcutaneously Q2W. In some embodiments, each dose is administered subcutaneously Q4W (i.e. once a month).

In some embodiments, the pharmaceutical composition (including pharmaceutical compositions comprising any of the TACI-Fc fusion proteins described herein) is administered to a subject via intravenous administration. In some embodiments, the dose of the TACI-Fc fusion for intravenous administration is at or about 2.4 mg. In some embodiments, the dose of the TACI-Fc fusion for intravenous administration is at or about 8 mg. In some embodiments, the dose of the TACI-Fc fusion for intravenous administration is at or about 24 mg. In some embodiments, the dose of the TACI-Fc fusion for intravenous administration is at or about 80 mg. In some embodiments, the dose of the TACI-Fc fusion for intravenous administration is at or about 240 mg. In some embodiments, the dose of the TACI-Fc fusion for intravenous administration is at or about 480 mg. In some embodiments, the dose of the TACI-Fc fusion for intravenous administration is at or about 720 mg. In some embodiments, each dose is administered intravenously Q1W. In some embodiments, each dose is administered intravenously Q2W. In some embodiments, each dose is administered intravenously Q4W (i.e. once a month).

In some embodiments, the pharmaceutical composition (including pharmaceutical compositions comprising any of the TACI-Fc fusion proteins described herein) is administered parenterally. In some embodiments, the pharmaceutical composition is in a form suitable for infusion injection, for example by intravenous injection. In some embodiments, the infusion duration is, is at least, or is about 30 minutes, 40 minutes, 50 minutes, 1 hour, 1.5 hours, 2 hours, 3 hours, 4 hours, 5 hours or 6 hours. In some embodiments the infusion duration is between about 30 minutes and 6 hours. In some embodiments, the infusion duration is between about 30 minutes and 5 hours. In some embodiments, the infusion duration is between about 30 minutes and 4 hours. In some embodiments, the infusion duration is between about 30 minutes and 3 hours. In some embodiments, the infusion duration is between about 30 minutes and 2 hours. In some embodiments, the infusion duration is between about 30 minutes and 1 hour. In some embodiments, the infusion duration is or is about 30 minutes.

In some embodiments, a dosing regimen may include intravenous and subcutaneous dosing. In some embodiments, an initial loading dose may be administered intravenously, followed by a maintenance dose(s) administered subcutaneously. In some embodiments, there is provided a load/maintenance regimen, in which an intravenous dose is given one time, followed by a subcutaneous dose on the same day, and then followed by administration of administration of maintenance doses subcutaneously once a week to once every three weeks. In some embodiments, the maintenance dose is administered once a week (Q1W). In some embodiments, the maintenance dose is administered once every two weeks (Q2W). In some embodiments, the maintenance dose is administered once a month (Q4W). In some embodiments, the maintenance dose is administered once every three months (Q12W).

In some embodiments, the dosing regimen may also include an intermediate/step down regimen in which the dose amount and/or frequency of administration is reduced over time. In some embodiment, the immunomodulatory protein (e.g. TACI-Fc fusion protein) is administered once a week (Q1W) for four weeks, and then is administered once a month (Q4W). In some embodiments, the immunomodulatory protein (e.g. TACI-Fc fusion protein) is administered once a week (Q1W) for four weeks, and then is administered once a week (Q1W) or once every two weeks (Q2W) for two to four weeks, and then is administered once every three months (Q12W).

In some embodiments, the dosing regimen may also include an intermediate/step down regimen in which the dose amount and/or frequency of administration is reduced over time. In some embodiment, the immunomodulatory protein (e.g. TACI-Fc fusion protein) is administered once a week (Q1W) for three to four doses, and then is administered once a month (Q4W). In some embodiments, the immunomodulatory protein (e.g. TACI-Fc fusion protein) is administered once a week (Q1W) for three four doses, and then is administered once a week (Q1W) or once every two weeks (Q2W) for two to four weeks, and then is administered once every three months (Q12W). In some embodiment, the immunomodulatory protein (e.g. TACI-Fc fusion protein) is administered once every other week (Q2W) for three to four doses, and then is administered once a month (Q4W). For instance, in some embodiments, the Q1W or Q2W dose is given for 3-4 doses then monthly (Q4W) at that dose or a higher dose. In some embodiments, at or about 80 mg is administered Q1W or Q2W for 3-4 doses and then monthly (Q4W) at that dose or a higher dose (e.g. 160 mg or 240 mg).

In some embodiments, the administration of the provided immunomodulatory protein, such as TACI-Fc fusion protein, in accord with the provided methods continues for a desired time as determined by a treating physician or investigator. In some embodiments, the administration is continued until the subject exhibits a complete response or clinical remission. In some embodiments, the administration of the provided immunomodulatory protein, such as TACI-Fc fusion protein, in accord with the provided methods continues for a treatment period. In some embodiments, the treatment period is for at or about 6 months to 3 years, such as at or about 24 weeks, 36 weeks, 48 weeks, 1 year (e.g. 52 weeks), 2 years or 3 years. In some embodiments, the administration is continued until such time as the subject's symptoms are worsening or the disease or condition has progressed or relapsed in the subject following a remission.

In some embodiments, the pharmaceutical composition is administered as a monotherapy (i.e., as a single agent) or as a combination therapy (i.e., in combination with one or more additional immunosuppressant agents). In some embodiments, the additional agent is a glucocorticoid (e.g., prednisone, dexamethasone, and hydrocortisone), cytostatic agent, such as a cytostatic agent that affect proliferation of T cells and/or B cells (e.g., purine analogs, alkylating agents, or antimetabolites), an antibody (e.g., anti-CD20, anti-CD25 or anti-CD3 monoclonal antibodies), cyclosporine, tacrolimus, sirolimus, everolimus, an interferon, an opioid, a TNF binding protein, mycophenolate, small biological agent, such as fingolimod or myriocin, cytokine, such as interferon beta-1a, an integrin agonist, or an integrin antagonist.

In some embodiments, the efficacy of the treatment is monitored in the subject. In some embodiments, the change in baseline over time in circulating levels of antibodies, such as autoantibodies are monitored in the subject.

In some embodiments, a clinical response is monitored in the subject.

In some embodiments of the methods, the treating can result in a clinical remission. In some aspects, the treating can result in a clinical remission without about 1 week, about 2 weeks, about 3 weeks, about 4 weeks, about 5 weeks, about 6 weeks, about 7 weeks, about 9 weeks, about 10 weeks, about 11 weeks, about 12 weeks, about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, about 46 weeks, about 48 weeks, about 50 weeks, about 52 weeks, about 54 weeks, about 56 weeks, about 58 weeks, about 60 weeks, about 62 weeks, about 64 weeks, about 66 weeks, about 68 weeks, about 70 weeks, about 72 weeks, about 74 weeks, about 76 weeks, about 78 weeks, or about 80 weeks from the first dose. In some embodiments, the treating results in a clinical remission within about 10 weeks from the first dose. In some embodiments, the treating results in a clinical remission within about 6 weeks from the first dose. In some embodiments, the treating results in a clinical remission at about 6 weeks from the first dose and at about 10 weeks from the first dose.

In some embodiments of any of the preceding methods, the clinical remission is a sustained remission. For example, in some embodiments, the sustained remission is a clinical remission at about 10 weeks, about 15 weeks, about 20 weeks, about 25 weeks, about 30 weeks, about 35 weeks, about 40 weeks, about 45 weeks, about 50 weeks, about 52 weeks, about 55 weeks, about 60 weeks, about 65 weeks, about 70 weeks, about 72 weeks, about 75 weeks, about 80 weeks, about 85 weeks, about 90 weeks, about 95 weeks, about 100 weeks, about 102 weeks, about 105 weeks, or about 110 weeks from the first dose. In some embodiments, the sustained remission is a clinical remission at about ten weeks from the first dose and at about 30 weeks from the first dose. In some embodiments, the sustained remission has a length of at least about 30 weeks, or at least about 7, about 8, about 9, about 10, about 11, or about 12 months. In some embodiments of any of the preceding aspects, the amelioration of one or more symptoms of the disease or condition, clinical remission, and/or clinical response is maintained at least one month (e.g., at least one month, at least two months, at least three months, at least four months, at least five months, at least six months, at least seven months, at least eight months, at least nine months, at least ten months, at least eleven months, at least twelve months, or longer) after the end of treatment.

### C. Exemplary Methods and Uses

In some embodiments, the provided methods and uses as described can be used in the treatment of an autoantibody-related disease or disorder and/or B-cell-related disease. In some embodiments, the methods and uses are for treating an autoantibody-related disease or disorder. Any of such diseases or disorders as described, including any described in Section VI.A, can be treated in accord with the provided methods and uses. In some embodiments, the methods and uses of the autoantibody-related disease or disorder, such as a B-cell related disease, can be by a dosing regimen as described in Section VI.B or VI.C. Exemplary methods and uses for administration of the provided pharmaceutical compositions containing an immunomodulatory protein, including TACI-Fc fusion proteins, as provided are described in the following subsections.

In some embodiments, the antibody-related disease or disorder is a rheumatic disease or disorder, a renal disease or disorder, a hematologic disease or disorder, a dermatologic disease or disorder, or a neurologic disease or disorder. The provided pharmaceutical compositions containing an immunomodulatory protein, including TACI-Fc fusion proteins as provided, can be used for the treatment of rheumatic disease, renal disease, hematologic disease, dermatologic disease or neurologic disease. In some embodiments, administration of the immunomodulatory proteins (e.g. TACI-Fc fusion protein) can specifically regulate B cell responses during the immune response. Additionally, administration of provided immunomodulatory proteins can be used to modulate B cell development, development of other cells, antibody production, and cytokine production. Administration or use of provided immunomodulatory proteins can also modulate B cell communication, such as by neutralizing the proliferative effects of BAFF or APRIL. In some embodiments, the provided immunomodulatory proteins can be used to block or neutralize the actions of B-cells in association with rheumatic disease, renal disease, hematologic disease, dermatologic disease or neurologic disease.

In some embodiments, administration or use of the provided immunomodulatory proteins (e.g., TACI Fc fusion) reduces the risk of the subject developing hypogammaglobulinemia. Hypogammaglobulinemia is a disorder caused by low serum immunoglobulin (e.g., IgG) or antibody levels, and encompasses a majority of immune-compromised patients. Huq et al., 2022 “Hypogainmaglobulinemia.” StatPearls [Internet] StatPearls Publishing. Hypogammaglobulinemia can be of primary or secondary origin. Secondary causes are usually induced by an external or acquired factor such as a corticosteroid or immunosuppressant drug, nutritional disorders, infections, chemotherapy, malignancy, nephrotic syndrome, other metabolic diseases, and hazardous environmental conditions. Hypogammaglobulinemia can be determined according due to diagnostic criteria known in the art, which can be defined by local standard practice(s). For example, diagnostic criteria for hypogammaglobulinemia by the European Society of Immunodeficiency (ESID) require a substantial decrease in IgG concentration defined by at least two standard deviations below the average for healthy adults, which is 8 to 12 g/L (Huq et al., StatPearls Publishing, 2023). Thus, in some embodiments, hypogammalgobulinemia is diagnosed by immunoglobulin levels ≤7 g/L, ≤6 g/L, ≤5 g/L, ≤4 g/L, ≤3 g/L, ≤2 g/L, or ≤1 g/L. IgG is the most prominent circulating immunoglobulin in both the vascular and extravascular compartments, and thus, IgG is crucial to diagnosing hypogammaglobulinemia. Reduced IgA and IgM are also seen with low IgG levels. In specific embodiments, hypogammalgobulinemia is diagnosed by IgG levels ≤7 g/L, ≤6 g/L, ≤5 g/L, ≤4 g/L, ≤3 g/L, ≤2 g/L, or ≤1 g/L. In specific embodiments, hypogammalgobulinemia is diagnosed by IgG levels ≤3 g/L. In specific embodiments, hypogammalgobulinemia is diagnosed by IgG levels ≤1.5 g/L.

In some embodiments, minimizing the risk of the subject developing hypogammaglobulinemia means that the provided TACI Fc fusion proteins do not cause a reduction in IgG≤7 g/L, ≤6 g/L, ≤5 g/L, ≤4 g/L, ≤3 g/L, ≤2 g/L, or ≤1 g/L in the subject. In some embodiments, minimizing the risk of the subject developing hypogammaglobulinemia means that the provided TACI Fc fusion proteins do not cause a reduction in ≤3 g/L. In some embodiments, minimizing the risk of the subject developing hypogammaglobulinemia means that the provided TACI Fc fusion proteins do not cause a reduction in ≤1.5 g/L.

In some embodiments, minimizing the risk of hypogammaglobulinemia occurs at the any of the doses or dose frequencies described in Section VI.B. In some embodiments, the immunomodulatory protein provided herein (e.g., TACI fusion) reduces risk of hypogammaglobulinemia when administered at 80 mg SC Q4W. In some embodiments, the immunomodulatory protein provided herein (e.g., TACI fusion) reduces risk of hypogammaglobulinemia when administered at 240 mg SC Q4W.

### 1. Systemic Lapas Erythematosas (SLE)

In some embodiments, the provided TACI-Fc fusion proteins can be used for treating rheumatic diseases like autoantibody-mediated systemic lupus erythematosus (SLE).

SLE is a relapsing, remitting heterogeneous systemic autoimmune disease with mild to life-threatening manifestations. The most common symptoms of SLE include low-grade fever, photosensitivity, oral ulcers, muscle aches, arthritis, fatigue, loss of appetite, rash, pleuritis, pericarditis, nephritis and poor circulation.

In rheumatic diseases like SLE and other autoantibody-related disorders, current treatments include polypharmacy and fail to achieve remission in the majority of patients. Agents targeting the B-cell cytokines B-cell activating factor (BAFF) and/or a proliferation-inducing ligand (APRIL), including the monoclonal antibody belimumab and the wild-type (WT) transmembrane activator and calcium modulating cyclophilin ligand interactor (TACI)-Fc fusion proteins atacicept and telitacicept, have demonstrated promising clinical potential in such diseases. However, there still exists a large unmet need as current biologics require large doses or produce severe adverse effects such as hypertension, dyslipidemia, new onset diabetes, etc. (see, e.g., Belimumab, Anifrolumab, and Voclosporin). This is because BAFF and APRIL support B cell development, differentiation, and survival. Co-neutralization of BAFF and APRIL dramatically reduces B cell function, including antibody function. Thus, any disease where B cells play a substantial role in disease initiation and progression can be treated with the TACI-Fc fusion proteins provided herein.

The TACI-Fc fusion protein provided herein not only provides a well-tolerated and effective inhibitor of immune responses in models of SLE, but the TACI-Fc fusion protein provided herein is a potentially best-in-class therapy targeting clinically validated cytokines with applicability in other autoimmune diseases, like glomerulonephritis, cytopenia or blistering skin disease. Moreover, the TACI-Fc fusion protein provided herein provides a substantial improvement in patient convenience as dosing may be performed once every four weeks (Q4W) subcutaneously.

In some aspects, provided herein is a method of treating Systemic lupus erythematosus (SLE) comprising administering the TACI-Fc fusion proteins provided herein. In some embodiments, the method comprises: a) selecting a subject for administration of a TACI-Fc fusion protein that has been diagnosed with SLE; and b) administering to the selected subject the TACI-Fc fusion protein in accord with provided methods and doses (e.g. described in Section IV.B), wherein the TACI-Fc fusion protein is a homodimer of two polypeptides of the formula TACI-linker-Fc, wherein TACI is a variant TACI polypeptide such as any as provided herein. In some embodiments, the variant TACI polypeptide comprises the amino acid substitutions K77E, F78Y and Y102D in the amino acid sequence set forth in SEQ ID NO:13 In some embodiments, the TACI-Fc fusion protein is administered subcutaneously at a dose of from at or about 80 mg to at or about 480 mg once every four weeks.

In some embodiments, the TACI-Fc fusion protein is administered at a dose that includes any dose as described in Section VI.B. In some embodiments, the dose is from at or about 80 mg to at or about 240 mg. In some embodiments, the dose is at or about 80 mg. In some embodiments, the dose is at or about 240 mg.

In some embodiments, the TACI-Fc fusion protein is administered once every four weeks (Q4W). In some embodiments, the dose is from at or about 80 mg to at or about 240 mg (Q4W). In some embodiments, the dose is at or about 80 mg Q4W. In some embodiments, the dose is at or about 240 mg Q4W. In some embodiments, the dose is from at or about 80 mg to at or about 960 mg Q4W. In some embodiments, the dose is at or about 960 mg Q4W.

In some embodiments, the SLE is mild to moderate SLE or moderate to severe SLE. In some embodiments, the subject is selected for treatment if at the time of screening the subject has active SLE for 6 months. In some embodiments, the subject having SLE is selected for treatment based on the level of protein in the urine as an indication of proteinuria. In some embodiments, the protein in the urine is urine total protein to creatinine ratio (UPCR; g/g). In some embodiments, the subject is selected for treatment if at the time of screening the SLE is characterized by one or more of the following: (i) a hybrid SELENA-SLEDAI score ≥8 or a hybrid SELENA-SLEDAI ≥6 if there is high anti-dsDNA or low complement (C) levels; (ii) ≤6 g/g urine total protein to creatinine ratio (proteinuria); (iii) A grade in the BILAG score in ≥1 organs; (iv) B grade in the BILAG score in ≥2 organs; and (v) Physicians Global Assessment (PGA) score ≥1.0. In some embodiments, the SLE is mild to moderate. In some embodiments, the SLE is moderate to severe. In some embodiments, a subject has moderate to severe SLE if the subject has active SLE for ≥6 months. In some embodiments, a subject has moderate to severe SLE if the subject's SLE is characterized by one or more of the following: (i) a hybrid SELENA-SLEDAI score ≥8 or a hybrid SELENA-SLEDAI ≥6 if there is high anti-dsDNA or low complement (C) levels; (ii) ≤6 g/g urine total protein to creatinine ratio (proteinuria); (iii) A grade in the BILAG score in ≥1 organs; (iv) B grade in the BILAG score in ≥2 organs; and (v) Physicians Global Assessment (PGA) score ≥1.0. In some embodiments, a subject with moderate to severe SLE is selected for treatment. In some embodiments, the subject having moderate to severe SLE is selected for treatment if the subject has active SLE for ≥6 months. In some embodiments, the subject having moderate to severe SLE is selected for treatment if the subject has SLE characterized by one or more of the following: (i) a hybrid SELENA-SLEDAI score ≥8 or a hybrid SELENA-SLEDAI ≥6 if there is high anti-dsDNA or low complement (C) levels; (ii) ≤6 g/g urine total protein to creatinine ratio (proteinuria); (iii) A grade in the BILAG score in ≥1 organs; (iv) B grade in the BILAG score in ≥2 organs; and (v) Physicians Global Assessment (PGA) score ≥1.0.

In some embodiments, the subject is receiving standard therapy for treating the SLE. In some embodiments, the subject is selected for treatment if the at the time of screening or the time of administering the TACI-Fc fusion protein, the subject is receiving a stable standard treatment regimen characterized by the stable use of a standard therapy for treating the SLE. In some embodiments the stable use is stable use of the standard therapy for at least 30 days.

In some embodiments, the TACI-Fc fusion protein is administered to the subject in combination with a standard therapy for treating the SLE. In some embodiments, the standard therapy comprises one of more of a corticosteroid, antimalarial (e.g. hydroxychloroquine), a non-steroidal anti-inflammatory drug (NSAID), or an immunosuppressant or immunomodulator, or any combination thereof. In some embodiments, the immunosuppressant or immunomodulator is selected from the group consisting of including azathioprine, mycophenolate (e.g. mycophenolate mofetil or sodium mycophenolate), cyclophosphamide, methotrexate, leflunomide, tacrolimus, cyclosporine and combinations of any of the foregoing. In some embodiments, the mycophenolate comprises mycophenolic acid (MPA).

In some embodiments, the standard therapy comprises a corticosteroid and administration of the corticosteroid is tapered after administering the TACI-Fc fusion protein. In some embodiments, the subject is selected for treatment if at the time of screening the subject is characterized by one or more of the following: (i) severe lupus nephritis. In some embodiments severe lupus nephritis is such as defined as urine protein >6 g/24 hours or serum creatinine >2.5 mg/dL or 221 μmol/L; (ii) required hemodialysis; (iii) received high-dose corticosteroids for ≥14 days in the last 2 months, such as wherein the high-dose corticosteroid is treatment with prednisone>100 mg/day or equivalent; and (iv) central nervous system disease caused by SLE or not caused by SLE in the last 2 months. In some embodiments, the central nervous system disease is epilepsy, psychosis, organic brain syndrome, cerebrovascular accident, encephalitis, or central nervous system vasculitis.

In some embodiments, the autoantibody-related disease or disorder is a renal (kidney) disease or disorder. In some embodiments, the autoantibody-related disease or disorder is a Glomerulonephritis.

In some embodiments, the TACI-Fc fusion protein is administered to the subject at the dosing regimen for at least 12-weeks, 16-weeks, 20-weeks, 24-weeks, 28-weeks, 32-weeks, 36-weeks, 40-weeks, 44-weeks, 48-weeks, 52-weeks, 56 weeks, 60 weeks, 64 weeks, 68 weeks, 72 weeks. In some embodiments, the TACI-Fc fusion protein is administered to the subject at the dosing regimen for about 2-weeks to about 2 years, such as from 12-weeks, 16-weeks, 20-weeks, 24-weeks, 28-weeks, 32-weeks, 36-weeks, 40-weeks, 44-weeks, 48-weeks, 52-weeks, or any value time between any of the foregoing. In some embodiments, the period of time is for at or about 28-weeks. In some embodiments, the administration regimen continues, such as at the direction of a medical provider or clinician, for example until disease progresses.

In some embodiments, the TACI-Fc fusion protein is administered to the subject Q4W for between 12 weeks and 72 weeks. In some embodiments, the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

In some embodiments, the TACI-Fc fusion protein is administered to the subject Q4W for 12 weeks. In some embodiments, the TACI-Fc fusion protein is administered to the subject Q4W for 16 weeks. In some embodiments, the TACI-Fc fusion protein is administered to the subject Q4W for 24 weeks. In some embodiments, the TACI-Fc fusion protein is administered to the subject Q4W for 48 weeks.

In some embodiments, the variant TACI polypeptide is set forth in SEQ ID NO:26.

In some embodiments, the linker is a GS linker of between 5 and 20 amino acids in length. In some embodiments, the linker is selected from GSGGS (SEQ ID NO: 76), GGGGS (G4S; SEQ ID NO: 77), GSGGGGS (SEQ ID NO: 74), GGGGSGGGGS (2×GGGGS; SEQ ID NO: 78), GGGGSGGGGSGGGGS (3×GGGGS; SEQ ID NO: 79), GGGGSGGGGSGGGGSGGGGS (4×GGGGS, SEQ ID NO:84), GGGGSGGGGSGGGGSGGGGSGGGGS (5×GGGGS, SEQ ID NO: 91), GGGGSSA (SEQ ID NO: 80), or GSGGGGSGGGGS (SEQ ID NO:194) or combinations thereof. In some embodiments, the linker is set forth in SEQ ID NO: 74.

In some embodiments, the Fc is an IgG1 Fc domain. In some embodiments, the Fc is a variant IgG1 Fc that exhibits reduced binding affinity to an Fc receptor and/or reduced effector function as compared to a wild-type IgG1 Fc domain. In some embodiments, the variant IgG1 Fc domain comprises one or more amino acid substitutions selected from L234A, L234V, L235A, L235E, G237A, S267K, R292C, N297G, and V302C, by EU numbering. In some embodiments, the variant IgG1 Fc comprises the amino acid substitutions L234A, L235E, and G237A by EU numbering. In some embodiments, the Fc comprises the amino acid substitution C220S, wherein the residues are numbered according to the EU index of Kabat. In some embodiments, the Fc lacks the hinge sequence EPKSS or EPKSC. In some embodiments, the Fc region comprises K447del, wherein the residue is numbered according to the EU index of Kabat.

In some embodiments, the Fc comprises the amino acid sequence set forth in SEQ ID NO:73. In some embodiments, the TACI-Fc fusion protein is set forth in SEQ ID NO: 167.

In some embodiments, the Fc comprises the amino acid sequence set forth in SEQ ID NO:81. In some embodiments, the TACI-Fc fusion protein is set forth in SEQ ID NO: 168.

In some embodiments, the TACI-Fc fusion protein is provided in a formulation comprising an acetic acid buffer having a pH of from about 4.0 to about 6.0, proline at a concentration of from at or about 1% to about 10%, and a surfactant at a concentration of from about 0.005 to about 0.05% (w/v). In some embodiments, the formulation has a pH of about 5.2. In some embodiments, the acetic acid buffer comprises a concentration of acetate of from at or about 5 mM to at or about 15 mM. In some embodiments, the acetic acid buffer comprises a concentration of acetate of at or about 10 mM. In some embodiments, the proline is at a concentration of about 2% to about 5%. In some embodiments, the proline is at a concentration of at or about 3%. In some embodiments, the surfactant is at a concentration of from about 0.01 to about 0.025% (w/v), such as at or about 0.015% (w/v). In some embodiments, the surfactant is polysorbate 80. In some embodiments, the amount of TACI-Fc fusion protein in the formulation is from about 50 mg to about 100 mg. In some embodiments, the amount of TACI-Fc fusion protein in the formulation is at or about 80 mg. In some embodiments, the concentration of the TACI-Fc fusion protein is between about 50 mg/mL and about 200 mg/mL. In some embodiments, the concentration of the TACI-Fc fusion protein is at or about 100 mg/mL.

In some embodiments, the TACI-Fc fusion protein is administered intravenously. In some embodiments, the TACI-Fc fusion protein is administered subcutaneously.

In some embodiments, a B cell immune response or activity is reduced in the subject. In some embodiments, the numbers of mature and total circulating B cells is reduced in the subject. In some embodiments, circulating serum immunoglobulins are reduced in the subject. In some embodiments, one or more of B cell maturation, differentiation, and/or proliferation is reduced or inhibited. In some embodiments, B cell immune response or activity is assessed by measuring immunoglobulins secreted by B cells (e.g., IgA, IgG, IgM, and/or IgE). In some embodiments, B cell maturation, differentiation, and/or proliferation is assessed by immunophenotyping B cells. In some embodiments, B cell maturation, differentiation, and/or proliferation is assessed by measuring immunoglobulins secreted by B cells (e.g., IgA, IgG, IgM, and/or IgE).

In some embodiments, circulating levels of an APRIL or BAFF protein are reduced in the subject. In some embodiments, circulating levels of free APRIL (i.e., unbound APRIL) are reduced in the subject. In some embodiments, circulating levels of free BAFF (i.e., unbound BAFF) are reduced in the subject. In some embodiments, the APRIL or BAFF protein is an APRIL homotrimer, BAFF homotrimer, APRIL/BAFF heterotrimer, or BAFF 60mer. In some embodiments, the subject is a human. In some embodiments, the subject is an adult subject, such as 18 years of age or older, for example 18-65 years of age.

### 2. Nephrology and Renal (Kidney) Disease

In some embodiments, the provided TACI-Fc fusion proteins can be used in the nephrology-based methods of treatment for treating autoantibody-mediated kidney disease. In some embodiments, the provided TACI-Fc fusion proteins can be used to treat an autoantibody-associated glomerular disease (also known as glomerulonephritis). In some embodiments, the autoantibody-associated glomerular disease may include immunoglobulin (Ig) A nephropathy (IgAN), lupus nephritis (LN), primary membranous nephropathy (pMN), or renal anti-neutrophil cytoplasmic antibody (ANCA)-associated vasculitis (AAV). In some embodiments, the autoantibody-associated glomerular disease may include focal segmental glomerulosclerosis (FSGS). In some embodiments, the autoantibody-associated glomerular disease may include minimal change disease (MCD).

Therapeutic agents targeting the B-cell cytokines BAFF and/or APRIL have demonstrated promising clinical potential in autoantibody-related GN such as LN and IgAN, and other B-cell-related diseases such as systemic lupus erythematosus; however, there is still need for more safe and efficacious therapies. TACI-Fc fusion protein provided herein mediates more potent inhibitory activity than WT TACI-Fc or BAFF- or APRIL-specific antibodies. In preclinical studies, the TACI-Fc fusion protein provided herein demonstrated enhanced PK and immunomodulatory properties vs. WT TACI-Fc, which may translate to lower and/or less frequent doses in human subjects. The TACI-Fc fusion protein provided herein also suppressed autoantibodies, renal IgG deposition, and nephritis in mouse models. Thus, the provided TACI-Fc fusion protein may significantly improve clinical outcomes in GN and other B-cell-related diseases.

In some embodiments, the provided immunomodulatory proteins (e.g. TACI-Fc) can be used to treat immunoglobulin (Ig) A nephropathy (IgAN). In some embodiments, the IgAN diagnosis has been confirmed by biopsy within less than or equal to 3 years prior to screening or selection for administration, or initiation of administration, of the TACI-Fc fusion protein treatment. In some embodiments, a biopsy may be carried out on the subject prior to administration of the TACI-Fc fusion protein. In some embodiments, the subject is one that has elevated galactose deficient IgAQ1 (Gd-IgAQ1) antibodies at the time of, or when selected for, administration with the TACI-Fc fusion protein. In some embodiments, the TACI-Fc fusion protein provided herein may reduce inflammation and harmful antibodies that damage kidneys in subjects with IgAN. In some embodiments, the TACI-Fc fusion protein provided herein may reduce or reverse kidney damage associated with IgAN.

In some embodiments, the provided immunomodulatory proteins (e.g. TACI-Fc) can be used to treat lupus nephritis (LN). In some embodiments, the LN diagnosis has been confirmed by biopsy within less than or equal to 1 year prior to the initiation of screening for administration, or initiation of administration, of the TACI-Fc fusion protein treatment. In some embodiments, the subject is one that has a renal biopsy that shows evidence of active, proliferative Class III or IV LN per ISN/RPS criteria (see e.g. Markowitz and D'Agati, 2007, Kidney Int. 71:491-5). In some embodiments, the subject may co-exhibit Class V disease in addition to either Class III or Class IV disease. In some embodiments, a biopsy may be carried out on the subject prior to administration of the TACI-Fc fusion protein. In some embodiments, the subject has elevated anti-double stranded DNA (anti-dsDNA) at the time of, or when selected for, administration with the TACI-Fc fusion protein. In some embodiments, the subject is positive for anti-nuclear antibody (ANA) at the time of, or when selected for, administration with the TACI-Fc fusion protein. In some embodiments, the subject that is positive for ANA has a titer of greater than or equal to 1:80. In some embodiments, the subject that is positive for anti-dsDNA has a tier of greater than or equal to 30 IU/mL. In some embodiments, the TACI-Fc fusion protein provided herein may reduce inflammation and harmful antibodies that damage kidneys in subjects with LN. In some embodiments, the TACI-Fc fusion protein provided herein may reduce or reverse kidney damage associated with LN.

In some embodiments, the provided immunomodulatory proteins (e.g. TACI-Fc) can be used to treat primary membranous nephropathy (pMN). In some embodiments, the pMN diagnosis has been confirmed by biopsy within less than or equal to 3 years prior to screening or selection for administration, or initiation of administration, of the TACI-Fc fusion protein treatment. In some embodiments, a biopsy may be carried out on the subject prior to administration of the TACI-Fc fusion protein. In some embodiments, the subject is positive for anti-phospholipase A2 receptor 1 (anti-PLA2R1) antibodies and/or anti-thrombospondin type-1 domain-containing 7A (anti-THSD7A) antibodies at the time of, or when selected for, administration with the TACI-Fc fusion protein. In some embodiments, the subject having SLE is selected for treatment based on the level of protein in the urine as an indication of proteinuria. In some embodiments, the protein in the urine is urine total protein to creatinine ratio (UPCR; g/g). In some embodiments, the subject is one that has less than 50% reduction of proteinuria in the last 24 weeks while on angiotensin-converting enzyme (ACE)/angiotensin receptor blockade (ARB). In some embodiments, the subject has received maximal ACE/ARB therapy for ≥12 weeks prior to screening or selection for administration, or initiation of administration, with stable blood pressure (BP) therapy. In some embodiments, a stable blood pressure comprises a normal blood pressure. In some embodiments, a normal blood pressure comprises less than or equal to 120/80 mmHg. In some embodiments, a normal blood pressure comprises less than or equal to 140/70 mmHg. In some embodiments, the subject is not receiving any concomitant medications that are considered prohibited in connection with administration of a TACI-Fc fusion protein, such as determined by a clinician or physician. In some embodiments, the TACI-Fc fusion protein provided herein may reduce inflammation and harmful antibodies that damage kidneys in subjects with pMN. In some embodiments, the TACI-Fc fusion protein provided herein may reduce or reverse kidney damage associated with pMN.

In some embodiments, the subject to be selected or treated is one that has not had a prior diagnosis of, or fulfills diagnostic criteria for, another glomerular disease, has an eGFR<30 mL/min/1.73m2 or rapidly progressive glomerulonephritis, has recent serious or ongoing infection, and/or risk or history of serious infection.

In some embodiments, the provided immunomodulatory proteins (e.g. TACI-Fc) can be used to treat renal anti-neutrophil cytoplasmic antibody (ANCA)-associated vasculitis (AAV). In some embodiments, the renal AAV diagnosis has been confirmed by biopsy within less than or equal to 23 years prior to screening or selection for administration, or initiation of administration, of the TACI-Fc fusion protein treatment. In some embodiments, the biopsy confirms evidence of renal ANCA-associated vasculitis. In some embodiments, a biopsy may be carried out on the subject prior to administration of the TACI-Fc fusion protein. In some embodiments the subject is positive for anti-proteinase 3 (PR3) or anti-myeloperoxidase (MPO) antibodies at the time of, or when selected for administration with the TACI-Fc fusion protein.

In some embodiments, the subject may receive standard of care (SOC) therapy for their underlying disorder, which is within the level of skill of the investigator or clinical physician. In some embodiments, the SOC therapy may include a renin-angiotensin-aldosterone system inhibitor (RAASi), a statin, a diuretic, an immune modulator, an immunosuppressant or a corticosteroid. In some embodiments, the subject has previously received the SOC therapy prior to receiving the provided TACI-Fc fusion protein. In some embodiments, the subject continues receiving the SOC therapy while receiving administration of the provided TACI-Fc fusion protein. In some embodiments, the SOC therapy is tapered over time after receiving administration of the provided TACI-Fc fusion protein. In some embodiments, the SOC therapy may include an antimalarial, an antibiotic such as a tetracycline, a steroid such as prednisone, a sodium-glucose cotransporter-2 (SGLT2) inhibitors, mycophenolate mofetil (MMF), mycophenolic acid (MPA), voclosporin or other SOC therapy within the level of a skilled artisan. In some embodiments, just prior to the initiation of administration of the TACI-Fc the subject has not received, or is not receiving, combination therapy with two immunomodulatory treatments, such as MMF and voclosporin.

In some embodiments, the subject has LN or renal AAV and the subject has received therapy with mycophenolate mofetil (MMF)/mycophenolic acid (MPA) or other immunotherapy as a standard of care therapy for treating the LN or renal AAV. In some embodiments, the subject is administered mycophenolate mofetil (MMF)/mycophenolic acid (MPA) or other immunotherapy as a standard of care therapy for treating the LN or renal AAV, prior to or during the administration of the provided TACI-Fc fusion protein. In some embodiments, the subject has not received a steroid within 5 days prior to the initiation of administration of the provided TACI-Fc fusion protein.

In some embodiments, the subject that is administered a provided immunomodulatory proteins (e.g. TACI-Fc) in accord with the provided methods does not have another renal disease including but not limited to diabetic nephropathy; C3 glomerulonephropathy; focal segmental glomerulosclerosis; thin basement membrane disease; Alport's disease; IgA vasculitis; minimal change disease; post-infectious glomerulonephritis; secondary membranous nephropathy (excluding LN Class V combined with Class III or IV); or secondary IgAN including but not limited to Celiac disease, Crohn's disease, HIV, or liver cirrhosis.

In some embodiments, the subject has not received an agent that directly depletes B lymphocytes (e.g. Rituximab) within 48 weeks prior to initiation of administration of the provided immunomodulatory proteins (e.g. TACI-Fc). In some embodiments, the subject may have received an agent that directly depletes B lymphocytes (e.g. Rituximab) within greater than 24 weeks prior to initiation of administration of the provided immunomodulatory proteins (e.g. TACI-Fc) if B cells have returned to normal reference ranges prior to administration of the TACI-Fc fusion protein.

In some embodiments, the subject has not received an agent that directly inhibits BAFF and/or APRIL, such as Belimumab, within 24 weeks prior to initiation of administration of the provided immunomodulatory proteins (e.g. TACI-Fc).

In some embodiments, the subject has not received administration of Intravenous Ig, abatacept, anifrolumab, belatacept, adalimumab, infliximab, certolizumab, etanercept, golimumab, anakinra, canakinumab, tocilizumab, sarilumab, satralizumab within 8 weeks prior to initiation of administration of the provided immunomodulatory proteins (e.g. TACI-Fc). In some embodiments, the subject has not received administration of any approved therapeutic agent for treating an immune disease within 8 weeks prior to initiation of administration of the provided immunomodulatory proteins (e.g. TACI-Fc).

In some embodiments, the subject has not received cyclophosphamide within 8 weeks prior to initiation of administration of the provided immunomodulatory proteins (e.g. TACI-Fc).

In some embodiments, the efficacy of the treatment is monitored in the subject. In some embodiments, the change in baseline over time in circulating levels of antibodies, such as autoantibodies are monitored in the subject.

In some embodiments, the subject has LN and a change from baseline over time of circulating levels of anti-dsDNA is monitored in the subject.

In some embodiments, the subject has IgAN and a change from baseline over time of circulating levels of Gd-IgA1 and anti-Gd-IgAQ1 is monitored in the subject. In some embodiments, the subject will have greater than a 50%, greater than a 55%, greater than a 60%, greater than a 65%, greater than a 70%, greater than a 75%, greater than an 80%, greater than a 85%, greater than a 90%, or greater than a 95% reduction in Gd-IgA1. In some embodiments, the subject will have greater than a 50% reduction in Gd-IgA1. In other embodiments, greater than 75%, greater than 80%, greater than 85%, greater than 90%, or greater than 95% of subjects receiving the TACI-Fc fusion protein will have a reduction in Gd-IgA1. In specific embodiments, greater than 75% of subjects receiving the TACI-Fc fusion protein will have greater than a 50% reduction in Gd-IgA1.

In some embodiments, the subject has pMN and a change from baseline over time of circulating levels of pMN and anti-MPO is monitored in the subject.

In some embodiments, the subject has renal AAV and a change from baseline over time of circulating levels of anti-PR3 is monitored in the subject.

In some embodiments, a change in baseline over time of a complement component is monitored in the subject. In some embodiments, the complement component is one or more of C3, C4 or CH50.

In some embodiments, a clinical response is monitored in the subject. In some embodiments, the clinical response may be assessed by monitoring baseline estimated glomerular filtration rate (eGFR) over time. In some embodiments, eGFR is calculated by an equation that uses serum creatine or cystatin C. In some embodiments, the eGFR is calculated by an equation that is independent of race, such as described in Inker et al., 2021 N Engl J Med., 385:1737-1749. In some embodiments, the eGFR may be estimated using the Chronic Kidney Disease Epidemiology Collaboration (CKD-EPI) formula.

In some embodiments, the response is measured as a renal response by determination of eGFR (e.g. using cystatin C race-independent equation). In some embodiments, the renal response is measured in subjects with LN or pMN.

In some embodiments, the subject has LN and complete renal response is present if the subject has UPCR less than 0.5 g/g (e.g. based on 24-hour urine collection) and eGFR is greater than or equal to the lower limit of normal (LLN) or there has been a less than 20% decrease in eGFR from baseline where the eGFR is less than LLN. In some embodiments, the subject has LN and a partial renal response is present if the subject has UPCR less than or equal to 3.5 g/g and a greater than 50% reduction from baseline (e.g. based on 24-hour urine collection), and eGFR is greater than or equal to 60 mL/min/1.73 m² or there is a less than a 20% decrease of eGFR from baseline.

In some embodiments, the subject has pMN and complete renal response is present if the subject has UPCR less than 0.3 g/g (e.g. based on 24-hour urine collection), serum albumin greater than 35 g/L, and eGFR is greater than or equal to 60 mL/min/1.73 m². In some embodiments, the subject has pMN and a partial renal response is present if the subject has UPCR less than or equal to 3.5 g/g and a greater than 50% reduction from baseline (e.g. based on 24-hour urine collection), serum albumin greater than 30 g/L, and stable eGFR (e.g. decline of less than 15% compared to baseline.

In some aspects, provided herein is a method of treating glomerulonephritis comprising administering the TACI-Fc fusion proteins provided herein. In some embodiments, the method comprises a) selecting a subject for administration of a TACI-Fc fusion protein that has been diagnosed with glomerulonephritis; and b) administering to the selected subject the TACI-Fc fusion protein in accord with provided methods and doses (e.g. as described in Section VI.B), wherein the TACI-Fc fusion protein is a homodimer of two polypeptides of the formula TACI-linker-Fc, wherein TACI is a variant TACI polypeptide such as any provided herein. In some embodiments, the variant TACI polypeptide comprises the amino acid substitutions K77E, F78Y and Y102D in the amino acid sequence set forth in SEQ ID NO:13. In some embodiments, TACI-Fc fusion protein is administered subcutaneously at a dose of from at or about 80 mg to at or about 480 mg once every four weeks.

In some embodiments, the TACI-Fc fusion protein is administered at a dose that includes any dose as described in Section VI.B. In some embodiments, the dose is from at or about 80 mg to at or about 240 mg. In some embodiments, the dose is at or about 80 mg. In some embodiments, the dose is at or about 240 mg.

In some embodiments, the TACI-Fc fusion protein is administered once every four weeks (Q4W). In some embodiments, the dose is from at or about 80 mg to at or about 240 mg (Q4W). In some embodiments, the dose is at or about 80 mg Q4W. In some embodiments, the dose is at or about 240 mg Q4W. In some embodiments, the dose is from at or about 80 mg to at or about 960 mg Q4W. In some embodiments, the dose is at or about 960 mg Q4W.

In some embodiments, the subject is selected for treatment if at the time of screening the subject has active Glomerulonephritis.

In some embodiments, the Glomerulonephritis is selected from the group consisting of IgA Nephropathy, Lupus Nephritis and Primary Membranous Nephropathy. In some embodiments, the subject is greater than or equal to (≥)18 years of age. In some embodiments, the subject having IgAN, LN or pMN is selected for treatment based on the level of protein in the urine as an indication of proteinuria. In some embodiments, the protein in the urine is urine total protein to creatinine ratio (UPCR; g/g). In some embodiments, the subject has received a proteinuria-lowering medication, such as combination of an ACE inhibitor (ACEI) and an ARB. In some embodiments, the proteinuria-lowering medication is titrated to the maximal dose that can be tolerated without adversely effecting systemic blood pressure GFR or serum potassium levels. In some embodiments, the stable maximal dose of a proteinuria-lowering medication, such as ACEI/ARB, has been administered for ≥12 weeks. In some embodiments, the subject has received stable background immunosuppression prior to treatment with the TACI-Fc. In some embodiments, the subject intends to maintain stable background immunosuppression through the treatment period with the TACI-Fc fusion protein. In some embodiments, the subject has not received any background immunosuppression (e.g. MMF≥1 g/day with or without corticosteroids) prior to treatment with the TACI-Fc, but optionally may receive stable calcineurin inhibitors (CnI).

In some embodiments, the Glomerulonephritis is IgA Nephropathy (IgAN) and is characterized by urine total protein to creatinine ratio (UPCR) greater than or equal to 0.5 g/g, such as greater than 0.6 g/g, 0.65 g/g, 0.70 g/g or 0.75 g/g UPCR (proteinuria). In some embodiments, the subject has received standard care of therapy, such as stable background immunosuppression. In some embodiments, the stable immunosuppression includes one or more of MMF (e.g. a stable dose of MMF of ≥1 g/day), corticosteroids, azathioprine (AZA), or calcineurin inhibitors (CnI). In some embodiments, the subject has not received standard of care therapy, such as has not received stable background immunosuppression.

In some embodiments, the subject having Glomerulonephritis, such as IgAN, LN or pMN, is not selected for treatment if at the time of screening the subject is characterized by one or more of the following: (i) eGFR<30 mL/min/1.73m2; (ii) rapidly progressive glomerulonephritis; (iii) sclerosis or interstitial fibrosis and tubular atrophy in >50% of the biopsy; (iv) receiving prior B cell depletion or BAFF/APRIL inhibitors for ≤24 weeks; (v) history of demyelinating disorder; and (vi) known immunodeficiency, including IgG<700 mg/dL, IgA<10 mg/dL, B cells <100 per μL, or CD4<200 per mm³.

In some embodiments, the Glomerulonephritis is IgA Nephropathy (IgAN) and the subject is selected for treatment if at the time of screening the subject is characterized by one or both of the following: (i) the subject was diagnosed (e.g., by biopsy) with IgA Nephropathy (IgAN)≤5 years prior to the screening; and (ii) the subject has ≥0.75 g/g urine total protein-creatine ratio (proteinuria).

In some embodiments, the Glomerulonephritis is IgAN and the subject is selected for treatment if at the time of screening the subject is characterized by one or both of the following: (i) the subject has been diagnosed (e.g., by biopsy) with IgA Nephropathy (IgAN)≤5 years prior to the screening; and (ii) the subject has ≥0.5 g/g urine total protein-creatine ratio (proteinuria); and the subject has not received stable background immunosuppression.

In some embodiments, the Glomerulonephritis is Lupus Nephritis and the Lupus Nephritis is Class III (active focal), Class IV (diffuse) or Class V (lupus membranous nephropathy). In some embodiments, the Lupus Nephritis is Class III-V. In some embodiments, the Lupus Nephritis is characterized by proteinuria greater than or equal to ≥1.0 g/g urine total protein to creatinine ratio (UPCR), such as UPCR≥2.0 g/g, UPCR≥2.5 g/g, UPCR≥3.0 g/g, UPCR≥3.5 g/g. In some embodiments, the subject is characterized by UPCR≥1.0 g/g with active urinary sediment. In some embodiments, the subject is characterized by UPCR≥3.5 g/g. In some embodiments, the subject has received standard care of therapy, such as stable background immunosuppression. In some embodiments, the subject has not received stable background immunosuppression (e.g. MMF 1 g/day).

In some embodiments, the Glomerulonephritis is Lupus Nephritis (LN) and the subject is selected for treatment if at the time of screening the subject is characterized by one or of the following: (i) the subject was diagnosed (e.g. biopsy) with Lupus Nephritis Class II-V≤3 years prior to the screening; (ii) UPCR≥1 g/g urine total protein-creatinine ratio (proteinuria); (iii) active urinary sediment; (iv) positive anti-dsDNA and antinuclear antibodies (ANA), such as wherein positive anti-dsDNA is a titer of ≥30 IU/mL and positive ANA is a titer of ≥1:80; and (v) received stable background immunosuppression, such as wherein the stable background immunosuppression is a stable dose of MMF of ≥1 g/day, with or without corticosteroids, for at least 8 weeks prior to the time of screening or the time of administering the TACI-Fc fusion protein. In some embodiments, the Glomerulonephritis is Lupus Nephritis (LN) and the subject is selected for treatment if at the time of screening the subject is characterized by
(i) the subject was diagnosed (e.g., by biopsy) with Lupus Nephritis Class III-V≤3 years prior to the screening;
(ii) UPCR≥1 g/g with active urinary sediment; biopsy ≤24 weeks; or UPCR≥3.5 g/g;
(iii) positive anti-dsDNA and antinuclear antibodies (ANA), such as wherein positive anti-dsDNA is a titer of ≥30 IU/mL and positive ANA is a titer of ≥1:80;
(iv) received stable background immunosuppression, such as wherein the stable background immunosuppression is a stable dose of MMF of ≥1 g/day, with or without corticosteroids; and
(v) is on a maintenance treatment such as with of MMF (e.g., of ≥1 g/day), azathioprine (AZA), or calcineurin inhibitors (CnI).

In some embodiments, the Glomerulonephritis is primary Membranous Nephropathy. In some embodiments, the Glomerulonephritis is primary Membranous Nephropathy (pMN) and the subject is selected for treatment if at the time of screening the subject is characterized by one or both of the following: (i) the subject was diagnosed with pMN≤5 years prior to the screening; and (ii) ≥3.5 g/g urine total protein to creatinine ratio (proteinuria); and (iii) positive anti-PLA2R1 or positive anti-THSD7A antibodies.

In some embodiments, the Glomerulonephritis is pMN and the subject is selected for treatment if at the time of screening the subject is characterized by one or more of the following:
(i) the subject was diagnosed (e.g. biopsy-confirmed) with pMN≤5 years prior to the screening;
(ii) the subject has UPCR≥3.5 g/g;
(iii) positive anti-PLA2R1 or positive anti-THSD7A antibodies;
(iv) less than 50% reduction of proteinuria in the past 24 weeks while on stable maximal dose of angiotensin-converting enzyme inhibitor (ACE)/angiotensin receptor blocker (ARB); and
(v) has not received stable background immunosuppression, except optionally may receive a stable dose of calcineurin inhibitor (CnI) for ≥12 weeks.

In some embodiments, the subject is selected for treatment if at the time of screening or the time of administering the TACI-Fc fusion protein the subject has received therapy with an Angiotensin-converting enzyme (ACE) inhibitor and/or angiotensin II receptor blocker (ARB), such as wherein the subject has received a maximally recommended dose of the ACE inhibitor or ARB therapy. In some embodiments, the subject is selected for treatment if at the time of screening or the time of administering the TACI-Fc fusion protein the subject has received therapy with an SGLT2 inhibitor. In some embodiments, the subject is selected for treatment if at the time of screening or the time of administering the TACI-Fc fusion protein the subject has received therapy with an endothelin receptor antagonist. In some embodiments, the subject is selected for treatment if at the time of screening or at the time of administering the TAC-Fc fusion protein the subject has a stable blood pressure.

In some embodiments, the autoantibody-related disease or disorder is a hematological disease or disorder. In some embodiments, the autoantibody-related disease or disorder is an autoimmune cytopenia.

In some embodiments, the TACI-Fc fusion protein is administered to the subject at the dosing regimen for at least 12-weeks, 16-weeks, 20-weeks, 24-weeks, 28-weeks, 32-weeks, 36-weeks, 40-weeks, 44-weeks, 48-weeks, 52-weeks. In some embodiments, the TACI-Fc fusion protein is administered to the subject at the dosing regimen for about 2-weeks to about 2 years, such as from 12-weeks, 16-weeks, 20-weeks, 24-weeks, 28-weeks, 32-weeks, 36-weeks, 40-weeks, 44-weeks, 48-weeks, 52-weeks, or any value time between any of the foregoing. In some embodiments, the period of time is for at or about 28-weeks. In some embodiments, the administration regimen continues, such as at the direction of a medical provider or clinician, for example until disease progresses.

In some embodiments, the TACI-Fc fusion protein is administered to the subject Q4W for between 12 weeks and 72 weeks. In some embodiments, the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

In some embodiments, the TACI-Fc fusion protein is administered to the subject Q4W for 12 weeks. In some embodiments, the TACI-Fc fusion protein is administered to the subject Q4W for 16 weeks. In some embodiments, the TACI-Fc fusion protein is administered to the subject Q4W for 24 weeks. In some embodiments, the TACI-Fc fusion protein is administered to the subject Q4W for 48 weeks.

In some embodiments, the variant TACI polypeptide is set forth in SEQ ID NO:26.

In some embodiments, the linker is a GS linker of between 5 and 20 amino acids in length. In some embodiments, the linker is selected from GSGGS (SEQ ID NO: 76), GGGGS (G4S; SEQ ID NO: 77), GSGGGGS (SEQ ID NO: 74), GGGGSGGGGS (2×GGGGS; SEQ ID NO: 78), GGGGSGGGGSGGGGS (3×GGGGS; SEQ ID NO: 79), GGGGSGGGGSGGGGSGGGGS (4×GGGGS, SEQ ID NO:84), GGGGSGGGGSGGGGSGGGGSGGGGS (5×GGGGS, SEQ ID NO: 91), GGGGSSA (SEQ ID NO: 80), or GSGGGGSGGGGS (SEQ ID NO:194) or combinations thereof. In some embodiments, the linker is set forth in SEQ ID NO: 74.

In some embodiments, the Fc is an IgG1 Fc domain. In some embodiments, the Fc is a variant IgG1 Fc that exhibits reduced binding affinity to an Fc receptor and/or reduced effector function as compared to a wild-type IgG1 Fc domain. In some embodiments, the variant IgG1 Fc domain comprises one or more amino acid substitutions selected from L234A, L234V, L235A, L235E, G237A, S267K, R292C, N297G, and V302C, by EU numbering. In some embodiments, the variant IgG1 Fc comprises the amino acid substitutions L234A, L235E, and G237A by EU numbering. In some embodiments, the Fc comprises the amino acid substitution C220S, wherein the residues are numbered according to the EU index of Kabat. In some embodiments, the Fc lacks the hinge sequence EPKSS or EPKSC. In some embodiments, the Fc region comprises K447del, wherein the residue is numbered according to the EU index of Kabat.

In some embodiments, the Fc comprises the amino acid sequence set forth in SEQ ID NO:73. In some embodiments, the TACI-Fc fusion protein is set forth in SEQ ID NO: 167.

In some embodiments, the Fc comprises the amino acid sequence set forth in SEQ ID NO:81. In some embodiments, the TACI-Fc fusion protein is set forth in SEQ ID NO: 168.

In some embodiments, the TACI-Fc fusion protein is provided in a formulation comprising an acetic acid buffer having a pH of from about 4.0 to about 6.0, proline at a concentration of from at or about 1% to about 10%, and a surfactant at a concentration of from about 0.005 to about 0.05% (w/v). In some embodiments, the formulation has a pH of about 5.2. In some embodiments, the acetic acid buffer comprises a concentration of acetate of from at or about 5 mM to at or about 15 mM. In some embodiments, the acetic acid buffer comprises a concentration of acetate of at or about 10 mM. In some embodiments, the proline is at a concentration of about 2% to about 5%. In some embodiments, the proline is at a concentration of at or about 3%. In some embodiments, the surfactant is at a concentration of from about 0.01 to about 0.025% (w/v), such as at or about 0.015% (w/v). In some embodiments, the surfactant is polysorbate 80. In some embodiments, the amount of TACI-Fc fusion protein in the formulation is from about 50 mg to about 100 mg. In some embodiments, the amount of TACI-Fc fusion protein in the formulation is at or about 80 mg. In some embodiments, the concentration of the TACI-Fc fusion protein is between about 50 mg/mL and about 200 mg/mL. In some embodiments, the concentration of the TACI-Fc fusion protein is at or about 100 mg/mL.

In some embodiments, the TACI-Fc fusion protein is administered intravenously. In some embodiments, the TACI-Fc fusion protein is administered subcutaneously.

In some embodiments, during the course of the treatment, the subject may be monitored for one or more endpoints of the disease to assess efficacy and/or one or more for an adverse event. In some embodiments, efficacy is monitored by an improvement in UPCR. In some embodiments, UPCR improves by at least or about 10% to 30%, 20% to 40%, 30% to 50%, 40% to 60%, 50% to 70%, 60% to 80%, 70% to 90%, or 80% to 100%. In some embodiments, the UPCR improves by at least or about 15%, 20%, 25%, 30%, or 35% compared to baseline. In some embodiments, the UPCR improves by at least or about 40%, 50%, 60%, 70% or 80% compared to baseline. In some embodiments, the UPCR improves by at least or about 25% compared to baseline. In some embodiments, the UPCR improves by at least or about 30%. In some embodiments, the UPCR improves by at least or about 40%. In some embodiments, the UPCR improves by at least or about 50%. In some embodiments, the UPCR improves by at least or about 70%. In some embodiments, the UPCR improves by at least or about 80%. In some embodiments, the UPCR improves by at least or about 100%.

In some embodiments, during the course of the treatment, the subject may be monitored for one or more endpoints of the disease to assess efficacy and/or one or more for an adverse event. In some embodiments, efficacy is monitored by a reduction in UPCR. In some embodiments, UPCR is reduced by at least or about 10% to 30%, 20% to 40%, 30% to 50%, 40% to 60%, 50% to 70%, 60% to 80%, 70% to 90%, or 80% to 100%. In some embodiments, the UPCR is reduced by at least or about 15%, 20%, 25%, 30%, or 35% compared to baseline. In some embodiments, the UPCR is reduced by at least or about 40%, 50%, 60%, 70% or 80% compared to baseline. In some embodiments, the UPCR is reduced by at least or about 25% compared to baseline. In some embodiments, the UPCR is reduced by at least or about 30%. In some embodiments, the UPCR is reduced by at least or about 40%. In some embodiments, the UPCR is reduced by at least or about 50%. In some embodiments, the UPCR is reduced by at least or about 70%. In some embodiments, the UPCR is reduced by at least or about 80%. In some embodiments, the UPCR reduced by at least or about 100%.

In some embodiments, the subject achieves complete remission as defined by a UPCR of <0.5 mg/mg.

In some embodiments, efficacy is monitored by a reduction in serum Ig levels. In some embodiments, serum Ig levels include, but are not limited to, serum IgA, serum IgG, and/or serum IgM levels. In some embodiments, serum Ig levels are reduced by at least or about 10% to 30%, 20% to 40%, 30% to 50%, 40% to 60%, 50% to 70%, 60% to 80%, 70% to 90%, or 80% to 100%. In some embodiments, serum Ig levels are reduced by at least or about 1%, 5%, 10%, 15%, 20%, 25%, 30%, 35%, 40%, 45%, 50%, 55%, 60%, 65%, 70%, 75%, 80%, 85%, or 90% compared to baseline. In some embodiments, serum Ig levels are reduced by at least or about 10%, 20%, 30%, 40%, 50%, 60%, 70%, 80%, or 90% compared to baseline. In some embodiments, serum Ig levels are reduced by at least or about 40%.

In some embodiments, the IgA comprises Gd-IgA1. In some embodiments, serum Gd-IgA1 levels are reduced by at least or about 10% to 30%, 20% to 40%, 30% to 50%, 40% to 60%, 50% to 70%, 60% to 80%, 70% to 90%, or 80% to 100%. In some embodiments, serum Gd-IgA1 levels are reduced by at least or about 1%, 5%, 10%, 15%, 20%, 25%, 30%, 35%, 40%, 45%, 50%, 55%, 60%, 65%, 70%, 75%, 80%, 85%, or 90% compared to baseline. In some embodiments, serum Gd-IgA1 levels are reduced by at least or about 10%, 20%, 30%, 40%, 50%, 60%, 70%, 80%, or 90%.

In some embodiments, efficacy is monitored by a reduction in serum anti-phospholipase A2 receptor (PLA2R). In some embodiments, serum anti-PLA2R levels are reduced by at least or about 1%, 5%, 10%, 15%, 20%, 25%, 30%, 35%, 40%, 45%, 50%, 55%, 60%, 65%, 70%, 75%, 80%, 85%, or 90% compared to baseline. In some embodiments, serum anti-PLA2R levels are reduced by at least or about 10%, 20%, 30%, 40%, 50%, 60%, 70%, 80%, or 90% compared to baseline. In some embodiments, serum anti-PLA2R levels are reduced by at least or about 30%. In some embodiments, serum anti-PLA2R levels are reduced by at least or about 40%.

In some embodiments, a B cell immune response or activity is reduced in the subject. In some embodiments, the numbers of mature and total circulating B cells is reduced in the subject. In some embodiments, circulating serum immunoglobulins are reduced in the subject. In some embodiments, one or more of B cell maturation, differentiation, and/or proliferation is reduced or inhibited. In some embodiments, B cell immune response or activity is assessed by measuring immunoglobulins secreted by B cells (e.g., IgA, IgG, IgM, and/or IgE). In some embodiments, B cell maturation, differentiation, and/or proliferation is assessed by immunophenotyping B cells. In some embodiments, B cell maturation, differentiation, and/or proliferation is assessed by measuring immunoglobulins secreted by B cells (e.g., IgA, IgG, IgM, and/or IgE).

In some embodiments, circulating levels of an APRIL or BAFF protein are reduced in the subject. In some embodiments, circulating levels of free APRIL (i.e., unbound APRIL) are reduced in the subject. In some embodiments, circulating levels of free BAFF (i.e., unbound BAFF) are reduced in the subject. In some embodiments, the APRIL or BAFF protein is an APRIL homotrimer, BAFF homotrimer, APRIL/BAFF heterotrimer, or BAFF 60mer. In some embodiments, the subject is a human. In some embodiments, the subject is an adult subject, such as 18 years of age or older, for example 18-65 years of age.

### 3. Hematologic Disease

In some embodiments, the provided TACI-Fc fusion proteins can be used for treating hematologic diseases like autoimmune cytopenia.

In preclinical studies, co-neutralization of BAFF and APRIL can directly suppress antibody-secreting cells (ASC) and reduce circulating immunoglobulins. Autoimmune hemolytic anemia (AIHA) and immune thrombocytopenic purpura (ITP) are cytopenia diseases characterized by autoantibodies directed against red blood cells and platelets, respectively. Significantly higher levels of both BAFF and APRIL have been observed in the serum of patients with AIHA and ITP compared to healthy subjects, and polymorphisms in BAFF and TACI have been associated with ITP (Abdel-Hamid et al. (2011) Am J Med; Emmerich et al. (2007) Br J Haematol; Peng et al. (2017) J Thromb Haemost; Yu et al. (2021) Blood Adv). Currently, there are no B cell targeting agents approved to treat AIHA or ITP. Rituximab, an anti-CD20 therapeutic that depletes CD20+ B cells and thereby only indirectly reduces autoantibody production, has demonstrated some efficacy in both ITP (Neunert et al. (2019) Blood) and AIHA (Jager et al. (2019) Blood Rev), though it is not approved for use in either indication. Belimumab, an anti-BAFF neutralizing antibody (Ab), has demonstrated encouraging efficacy in the treatment of ITP associated with systemic lupus erythematosus (SLE) (De Marchi et al. (2017) Clin Exp Rheumatol) and also in combination with rituximab in patients with ITP (Mahévas et al. (2021) Haematologica), but only had a modest impact on hematological manifestations in SLE patients (Manzi et al. (2012) Ann Rheum Dis.

The provided TACI-Fc fusion protein can address the shortcomings of existing B-cell-targeting agents, by substantially improving upon the ligand affinity liabilities of wild type (WT) TACI, resulting in highly potent dual BAFF/APRIL inhibition superior to WT TACI-Fc, or to BAFF- or APRIL-specific monoclonal Abs. In preclinical studies, the TACI-Fc fusion protein provided herein demonstrates enhanced pharmacokinetic (PK) and immunomodulatory properties vs. WT TACI-Fc, which may translate to lower and/or less frequent doses in human subjects. The provided TACI-Fc fusion protein also suppresses autoantibodies and nephritis in mouse models of lupus. Taken together, these observations suggest that a potent dual BAFF/APRIL antagonist, such as the TACI-fusion protein provided herein, can lower pathogenic autoantibody levels, and may be particularly beneficial in the treatment of antibody-related hematologic diseases, such as autoimmune cytopenias.

In some aspects, provided herein is a method of treating autoimmune cytopenia comprising administering the TACI-Fc fusion proteins provided herein. In some embodiments, the method comprises a) selecting a subject for administration of a TACI-Fc fusion protein that has been diagnosed with an autoimmune cytopenia; and b) administering to the selected subject the TACI-Fc fusion protein in accord with the provided methods and doses (e.g. as described in Section VI.B), wherein the TACI-Fc fusion protein is a homodimer of two polypeptides of the formula TACI-linker-Fc, wherein TACI is a variant TACI polypeptide such as any as provided herein. In some embodiments, the variant TACI polypeptide comprises the amino acid substitutions K77E, F78Y and Y102D in the amino acid sequence set forth in SEQ ID NO:13. In some embodiments, the TACI-Fc fusion protein is administered subcutaneously at a dose of from at or about 80 mg to at or about 480 mg once every four weeks.

In some embodiments, the TACI-Fc fusion protein is administered at a dose that includes any dose as described in Section VI.B. In some embodiments, the dose is from at or about 80 mg to at or about 240 mg. In some embodiments, the dose is at or about 80 mg. In some embodiments, the dose is at or about 240 mg. In some embodiments, the dose is from at or about 80 mg to at or about 960 mg Q4W. In some embodiments, the dose is at or about 960 mg Q4W.

In some embodiments, the TACI-Fc fusion protein is administered once every four weeks (Q4W). In some embodiments, the dose is from at or about 80 mg to at or about 240 mg (Q4W). In some embodiments, the dose is at or about 80 mg Q4W. In some embodiments, the dose is at or about 240 mg Q4W.

In some embodiments, the subject is selected for treatment if at the time of screening the subject has active cytopenia. In some embodiments, the autoimmune cytopenia is selected from the group consisting of Immune Thrombocytopenia (ITP) and Autoimmune Hemolytic Anemia (AIHA). In some embodiments, the subject is greater than or equal to 18 years of age. In some embodiments, the subject intends to maintain any background therapy through the treatment period. In some embodiments, the background therapy comprises corticosteroids (e.g., at a dose ≤20 mg), thrombopoietin receptor agonists (TPO-RA), erythropoiesis-stimulating agents (ESA), or oral immunosuppressants. In some embodiments, the cytopenia is ITP and is characterized by urine total protein to creatinine ratio (UPCR) greater than or equal to 2 g/g, such as greater than 2.5 g/g/, 3 g/g, 3.5 g/g or 4 g/g urine total protein to creatinine ratio (UPCR).

In some embodiments, the subject having autoimmune cytopenia, such as ITP, wAIHA or CAD, is not selected for treatment if at the time of screening the subject is characterized by one or more of the following: (i) secondary autoimmune cytopenia including Evans Syndrome; (ii) received prior B cell depletion (BCD) or BAFF/APRIL inhibitors for ≤12 weeks, however, BCD for 12 weeks is ok if B cells and Ig levels are normal; (iii) history of demyelinating disorder; and (iv) known immunodeficiency, including IgG<700 mg/DL, IgA<10 mg/dL, B cells <100 per μL, or CD4<200 per mm³.

In some embodiments, the autoimmune cytopenia is ITP and the subject is selected for treatment if at the time of screening the subject is characterized by one or more of the following:
(i) the subject was diagnosed with ITP≥3 months prior to the screening;
(ii) sustained low platelet counts (e.g., <30,000/μL); and
(iii) received ≥4 prior treatments for treating the ITP.

In some embodiments, the autoimmune cytopenia is ITP and the subject is selected for treatment if at the time of screening the subject is characterized by one or more of the following:
(i) the subject was diagnosed with ITP≥3 months prior to the screening;
(ii) sustained low platelet counts (e.g., <30,000/μL);
(iii) received ≥2 prior treatments for treating the ITP, including ≥1 TPO-RA and ≥prior response; and
(iv) no concomitant immunosuppression, except for corticosteroids (e.g., ≤20 mg).

In some embodiments, the TACI-Fc fusion protein provided herein may reduce harmful antibodies that attack and destroy platelets in subjects with ITP.

In some embodiments, the TACI-Fc fusion protein provided herein may reduce harmful antibodies that attack and destroy red blood cells in subjects with ITP.

In some embodiments, the autoimmune cytopenia is an AIHA and the AIHA is warm AIHA (wAIHA) or cold AIHA (cAIHA or cold agglutinin disease, CAD). In some embodiments, the cytopenia is wAIHA or CAD and is characterized by sustained hemoglobin greater than 9 g/dL such as greater than 9.5 g/dL or 10 g/dL.

In some embodiments, the autoimmune cytopenia is wAIHA or CAD and the subject is selected for treatment if at the time of screening the subject is characterized by one or more of the following:
(i) the subject was diagnosed with wAIHA or CAD≥3 months prior to the screening;
(ii) sustained hemoglobin (Hb)<9 g/dL;
(iii) received ≥2 prior treatments for treating the wAIHA or CAD; and
(iv) is experiencing symptoms from anemia.

In some embodiments, the autoimmune cytopenia is wAIHA or CAD and the subject is selected for treatment if at the time of screening the subject is characterized by one or more of the following:
(i) the subject was diagnosed with primary disease (wAIHA or CAD)≥12 weeks prior to the screening and ≥2 prior treatment regimens;
(ii) sustained hemoglobin (Hb)<10 g/dL;
(iii) received ≥2 prior treatments for treating the wAIHA or CAD;
(iv) is experiencing symptoms from anemia;
(v) IgG+ direct antiglobulin (DAT) test or cold agglutinin (CAD) titer; and
(vi) evidence of hemolysis by haptoglobin, iBili, or LDH.

In some embodiments, the autoimmune cytopenia is warm autoimmune hemolytic anemia (wAIHA).

In some embodiments, the autoimmune cytopenia is cold autoimmune hemolytic anemia (cAIHA or CAD).

In some embodiments, the TACI-Fc fusion protein provided herein may reduce harmful antibodies that attack and destroy platelets in subjects with CAD.

In some embodiments, the subject is selected for treatment if at the time of screening or the time of administering the TACI-Fc fusion protein, the subject is receiving a stable immunosuppression. In some embodiments, the TACI-Fc fusion protein is administered to the subject in combination with concurrent administration of the stable immunosuppression. In some embodiments, the stable immunosuppression comprises a stable dose of a steroid, such as a corticosteroid, for at least two weeks prior to the time of screening or the time of administering the TACI-Fc fusion protein; and/or the stable immunosuppression comprises a stable dose of azathioprine, MMF, or cyclosporine for at least four weeks prior to the time of screening or the time of administering the TACI-Fc fusion protein. In some embodiments, the stable immunosuppression comprises a calcineurin inhibitor (e.g., cyclosporine or tacrolimus) for at least four weeks prior to the time of screening or the time of administering the TACI-Fc fusion protein.

In some embodiments, the subject is not characterized by having a secondary cytopenia (e.g. systemic autoimmune disease or malignancy) or Evans syndrome. In some embodiments, the autoantibody-related disease or disorder is a dermatologic disease or disorder. In some embodiments, the autoantibody-related disease or disorder is an autoimmune bullous dermatosis.

In some embodiments, the TACI-Fc fusion protein is administered to the subject at the dosing regimen for at least 12-weeks, 16-weeks, 20-weeks, 24-weeks, 28-weeks, 32-weeks, 36-weeks, 40-weeks, 44-weeks, 48-weeks, 52-weeks, 56 weeks, 60 weeks, 64 weeks, 68 weeks, 72 weeks. In some embodiments, the TACI-Fc fusion protein is administered to the subject at the dosing regimen for about 2-weeks to about 2 years, such as from 12-weeks, 16-weeks, 20-weeks, 24-weeks, 28-weeks, 32-weeks, 36-weeks, 40-weeks, 44-weeks, 48-weeks, 52-weeks, or any value time between any of the foregoing. In some embodiments, the period of time is for at or about 28-weeks. In some embodiments, the administration regimen continues, such as at the direction of a medical provider or clinician, for example until disease progresses.

In some embodiments, the TACI-Fc fusion protein is administered to the subject Q4W for between 12 weeks and 72 weeks. In some embodiments, the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

In some embodiments, the TACI-Fc fusion protein is administered to the subject Q4W for 12 weeks. In some embodiments, the TACI-Fc fusion protein is administered to the subject Q4W for 16 weeks. In some embodiments, the TACI-Fc fusion protein is administered to the subject Q4W for 24 weeks. In some embodiments, the TACI-Fc fusion protein is administered to the subject Q4W for 48 weeks.

In some embodiments, the variant TACI polypeptide is set forth in SEQ ID NO:26.

In some embodiments, the linker is a GS linker of between 5 and 20 amino acids in length. In some embodiments, the linker is selected from GSGGS (SEQ ID NO: 76), GGGGS (G4S; SEQ ID NO: 77), GSGGGGS (SEQ ID NO: 74), GGGGSGGGGS (2×GGGGS; SEQ ID NO: 78), GGGGSGGGGSGGGGS (3×GGGGS; SEQ ID NO: 79), GGGGSGGGGSGGGGSGGGGS (4×GGGGS, SEQ ID NO:84), GGGGSGGGGSGGGGSGGGGSGGGGS (5×GGGGS, SEQ ID NO: 91), GGGGSSA (SEQ ID NO: 80), or GSGGGGSGGGGS (SEQ ID NO:194) or combinations thereof. In some embodiments, the linker is set forth in SEQ ID NO: 74.

In some embodiments, the Fc is an IgG1 Fc domain. In some embodiments, the Fc is a variant IgG1 Fc that exhibits reduced binding affinity to an Fc receptor and/or reduced effector function as compared to a wild-type IgG1 Fc domain. In some embodiments, the variant IgG1 Fc domain comprises one or more amino acid substitutions selected from L234A, L234V, L235A, L235E, G237A, S267K, R292C, N297G, and V302C, by EU numbering. In some embodiments, the variant IgG1 Fc comprises the amino acid substitutions L234A, L235E, and G237A by EU numbering. In some embodiments, the Fc comprises the amino acid substitution C220S, wherein the residues are numbered according to the EU index of Kabat. In some embodiments, the Fc lacks the hinge sequence EPKSS or EPKSC. In some embodiments, the Fc region comprises K447del, wherein the residue is numbered according to the EU index of Kabat.

In some embodiments, the Fc comprises the amino acid sequence set forth in SEQ ID NO:73. In some embodiments, the TACI-Fc fusion protein is set forth in SEQ ID NO: 167.

In some embodiments, the Fc comprises the amino acid sequence set forth in SEQ ID NO:81. In some embodiments, the TACI-Fc fusion protein is set forth in SEQ ID NO: 168.

In some embodiments, the TACI-Fc fusion protein is provided in a formulation comprising an acetic acid buffer having a pH of from about 4.0 to about 6.0, proline at a concentration of from at or about 1% to about 10%, and a surfactant at a concentration of from about 0.005 to about 0.05% (w/v). In some embodiments, the formulation has a pH of about 5.2. In some embodiments, the acetic acid buffer comprises a concentration of acetate of from at or about 5 mM to at or about 15 mM. In some embodiments, the acetic acid buffer comprises a concentration of acetate of at or about 10 mM. In some embodiments, the proline is at a concentration of about 2% to about 5%. In some embodiments, the proline is at a concentration of at or about 3%. In some embodiments, the surfactant is at a concentration of from about 0.01 to about 0.025% (w/v), such as at or about 0.015% (w/v). In some embodiments, the surfactant is polysorbate 80. In some embodiments, the amount of TACI-Fc fusion protein in the formulation is from about 50 mg to about 100 mg. In some embodiments, the amount of TACI-Fc fusion protein in the formulation is at or about 80 mg. In some embodiments, the concentration of the TACI-Fc fusion protein is between about 50 mg/mL and about 200 mg/mL. In some embodiments, the concentration of the TACI-Fc fusion protein is at or about 100 mg/mL.

In some embodiments, the TACI-Fc fusion protein is administered intravenously. In some embodiments, the TACI-Fc fusion protein is administered subcutaneously.

In some embodiments, a B cell immune response or activity is reduced in the subject. In some embodiments, the numbers of mature and total circulating B cells is reduced in the subject. In some embodiments, circulating serum immunoglobulins are reduced in the subject. In some embodiments, one or more of B cell maturation, differentiation, and/or proliferation is reduced or inhibited. In some embodiments, B cell immune response or activity is assessed by measuring immunoglobulins secreted by B cells (e.g., IgA, IgG, IgM, and/or IgE). In some embodiments, B cell maturation, differentiation, and/or proliferation is assessed by immunophenotyping B cells. In some embodiments, B cell maturation, differentiation, and/or proliferation is assessed by measuring immunoglobulins secreted by B cells (e.g., IgA, IgG, IgM, and/or IgE).

In some embodiments, circulating levels of an APRIL or BAFF protein are reduced in the subject. In some embodiments, circulating levels of free APRIL (i.e., unbound APRIL) are reduced in the subject. In some embodiments, circulating levels of free BAFF (i.e., unbound BAFF) are reduced in the subject. In some embodiments, the APRIL or BAFF protein is an APRIL homotrimer, BAFF homotrimer, APRIL/BAFF heterotrimer, or BAFF 60mer. In some embodiments, the subject is a human. In some embodiments, the subject is an adult subject, such as 18 years of age or older, for example 18-65 years of age.

### 4: Dermatologic Disease

In some embodiments, the provided TACI-Fc fusion proteins can be used for treating dermatologic diseases like autoimmune bullous (blistering) dermatosis (ABDs; also known as autoimmune blistering diseases). ABDs include Pemphigus vulgaris and Pemphigus foliaceus, among others. ABDs also include epidermolysis bullosa acquisita (EBA).

ABDs reflect autoantibodies targeting structural skin proteins. Treatments are limited to rituximab, which is the only biologic approved for pemphigus vulgaris, but may be associated with frequent relapses, often accompanied by elevations in the cytokine BAFF. BAFF and its related cytokine APRIL play key roles in B-cell activation across a broader spectrum of B cells than rituximab and are elevated in ABDs, correlating with disease activity. BAFF/APRIL inhibition may lead to more durable autoantibody reductions, improving clinical outcomes. The provided TACI-Fc fusion proteins provide an engineered BAFF/APRIL inhibitor that is more potent preclinically than other BAFF and/or APRIL inhibitors evaluated. Some are approved and/or demonstrate promising potential for related diseases like lupus, particularly its cutaneous manifestations.

In some aspects, provided herein is a method of treating autoimmune bullous (blistering) dermatosis (ABDs) comprising administering the TACI-Fc fusion proteins provided herein. In some embodiments, the method comprises: a) selecting a subject for administration of a TACI-Fc fusion protein that has been diagnosed with an autoimmune bullous (blistering) dermatosis; and b) administering to the selected subject the TACI-Fc fusion protein in accord with provided methods and doses (e.g. described in Section VI. B), wherein: the TACI-Fc fusion protein is a homodimer of two polypeptides of the formula TACI-linker-Fc, wherein TACI is a variant TACI polypeptide such as any as provided herein. In some embodiments, the variant TACI polypeptide comprises the amino acid substitutions K77E, F78Y and Y102D in the amino acid sequence set forth in SEQ ID NO:13. In some embodiments, the TACI-Fc fusion protein is administered subcutaneously at a dose of from at or about 80 mg to at or about 480 mg once every four weeks.

In some embodiments, the TACI-Fc fusion protein is administered at a dose that includes any dose as described in Section VI.B. In some embodiments, the dose is from at or about 80 mg to at or about 240 mg. In some embodiments, the dose is at or about 80 mg. In some embodiments, the dose is at or about 240 mg.

In some embodiments, the TACI-Fc fusion protein is administered once every four weeks (Q4W). In some embodiments, the dose is from at or about 80 mg to at or about 240 mg (Q4W). In some embodiments, the dose is at or about 80 mg Q4W. In some embodiments, the dose is at or about 240 mg Q4W. In some embodiments, the dose is from at or about 80 mg to at or about 960 mg Q4W. In some embodiments, the dose is at or about 960 mg Q4W.

In some embodiments, the subject is selected for treatment if at the time of screening the subject has active blistering disease. In some embodiments, the subject has active blistering disease if the subject has visible blisters.

In some embodiments, the autoimmune bullous (blistering) dermatosis is selected from the group consisting of Pemphigus vulgaris, Pemphigus foliaceus or Bullous Pemphigoid. In some embodiments, the autoimmune bullous (blistering) dermatosis is Pemphigus vulgaris or Pemphigus foliaceus and the subject is selected for treatment if at the time of screening the subject is characterized by one or both of the following: (i) a Pemphigus Disease Area Index (PDAI)≥15; and (ii) positive anti-Dsg1 or positive anti-Dsg3 antibodies.

In some embodiments, the autoimmune bullous (blistering) dermatosis is Pemphigus vulgaris.

In some embodiments, the autoimmune bullous (blistering) dermatosis is Pemphigus foliaceus.

In some embodiments, the autoimmune bullous (blistering) dermatosis is Pemphigoid and the subject is selected for treatment if at the time of screening the subject is characterized by one or both of the following: (i) IgA antibodies; and (ii) positive anti-Bp180 or positive anti-Bp230 antibodies.

In some embodiments, the subject is selected for treatment if at the time of screening or the time of administering the TACI-Fc fusion protein, the subject is receiving a stable immunosuppression. In some embodiments, the TACI-Fc fusion protein is administered to the subject in combination with concurrent administration of the stable immunosuppression.

In some embodiments, the stable immunosuppression comprises a stable dose of a steroid, such as a corticosteroid, for at least two weeks prior to the time of screening or the time of administering the TACI-Fc fusion protein; and/or the stable immunosuppression comprises a stable dose of azathioprine, MMF, or cyclosporine for at least four weeks prior to the time of screening or the time of administering the TACI-Fc fusion protein. In some embodiments, the stable immunosuppression comprises a stable dose of a calcineurin inhibitor (e.g., cyclosporine or tacrolimus) for at least four weeks prior to the time of screening or the time of administering the TACI-Fc fusion protein.

In some embodiments, the subject is not characterized by having a secondary disease (e.g. paraneoplastic). In some embodiments, the autoantibody-related disease or disorder is a neurologic disease or disorder. In some embodiments, the autoantibody-related disease or disorder is Encephalitis.

In some embodiments, the TACI-Fc fusion protein is administered to the subject at the dosing regimen for at least 12-weeks, 16-weeks, 20-weeks, 24-weeks, 28-weeks, 32-weeks, 36-weeks, 40-weeks, 44-weeks, 48-weeks, 52-weeks, 56 weeks, 60 weeks, 64 weeks, 68 weeks, 72 weeks. In some embodiments, the TACI-Fc fusion protein is administered to the subject at the dosing regimen for about 2-weeks to about 2 years, such as from 12-weeks, 16-weeks, 20-weeks, 24-weeks, 28-weeks, 32-weeks, 36-weeks, 40-weeks, 44-weeks, 48-weeks, 52-weeks, or any value time between any of the foregoing. In some embodiments, the period of time is for at or about 28-weeks. In some embodiments, the administration regimen continues, such as at the direction of a medical provider or clinician, for example until disease progresses.

In some embodiments, the TACI-Fc fusion protein is administered to the subject Q4W for between 12 weeks and 72 weeks. In some embodiments, the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

In some embodiments, the TACI-Fc fusion protein is administered to the subject Q4W for 12 weeks. In some embodiments, the TACI-Fc fusion protein is administered to the subject Q4W for 16 weeks. In some embodiments, the TACI-Fc fusion protein is administered to the subject Q4W for 24 weeks. In some embodiments, the TACI-Fc fusion protein is administered to the subject Q4W for 48 weeks.

In some embodiments, during the course of the treatment, the subject may be monitored for one or more endpoints of the disease to assess efficacy and/or one or more for an adverse event. In some embodiments, efficacy is monitored by disease control (DC). In some embodiments, efficacy is measured by partial remission (PR). In some embodiments, efficacy is monitored by complete remission (CR).

In some embodiments, the variant TACI polypeptide is set forth in SEQ ID NO:26.

In some embodiments, the linker is a GS linker of between 5 and 20 amino acids in length. In some embodiments, the linker is selected from GSGGS (SEQ ID NO: 76), GGGGS (G4S; SEQ ID NO: 77), GSGGGGS (SEQ ID NO: 74), GGGGSGGGGS (2×GGGGS; SEQ ID NO: 78), GGGGSGGGGSGGGGS (3×GGGGS; SEQ ID NO: 79), GGGGSGGGGSGGGGSGGGGS (4×GGGGS, SEQ ID NO:84), GGGGSGGGGSGGGGSGGGGSGGGGS (5×GGGGS, SEQ ID NO: 91), GGGGSSA (SEQ ID NO: 80), or GSGGGGSGGGGS (SEQ ID NO:194) or combinations thereof. In some embodiments, the linker is set forth in SEQ ID NO: 74.

In some embodiments, the Fc is an IgG1 Fc domain. In some embodiments, the Fc is a variant IgG1 Fc that exhibits reduced binding affinity to an Fc receptor and/or reduced effector function as compared to a wild-type IgG1 Fc domain. In some embodiments, the variant IgG1 Fc domain comprises one or more amino acid substitutions selected from L234A, L234V, L235A, L235E, G237A, S267K, R292C, N297G, and V302C, by EU numbering. In some embodiments, the variant IgG1 Fc comprises the amino acid substitutions L234A, L235E, and G237A by EU numbering. In some embodiments, the Fc comprises the amino acid substitution C220S, wherein the residues are numbered according to the EU index of Kabat. In some embodiments, the Fc lacks the hinge sequence EPKSS or EPKSC. In some embodiments, the Fc region comprises K447del, wherein the residue is numbered according to the EU index of Kabat.

In some embodiments, the Fc comprises the amino acid sequence set forth in SEQ ID NO:73. In some embodiments, the TACI-Fc fusion protein is set forth in SEQ ID NO: 167.

In some embodiments, the Fc comprises the amino acid sequence set forth in SEQ ID NO:81. In some embodiments, the TACI-Fc fusion protein is set forth in SEQ ID NO: 168.

In some embodiments, the TACI-Fc fusion protein is provided in a formulation comprising an acetic acid buffer having a pH of from about 4.0 to about 6.0, proline at a concentration of from at or about 1% to about 10%, and a surfactant at a concentration of from about 0.005 to about 0.05% (w/v). In some embodiments, the formulation has a pH of about 5.2. In some embodiments, the acetic acid buffer comprises a concentration of acetate of from at or about 5 mM to at or about 15 mM. In some embodiments, the acetic acid buffer comprises a concentration of acetate of at or about 10 mM. In some embodiments, the proline is at a concentration of about 2% to about 5%. In some embodiments, the proline is at a concentration of at or about 3%. In some embodiments, the surfactant is at a concentration of from about 0.01 to about 0.025% (w/v), such as at or about 0.015% (w/v). In some embodiments, the surfactant is polysorbate 80. In some embodiments, the amount of TACI-Fc fusion protein in the formulation is from about 50 mg to about 100 mg. In some embodiments, the amount of TACI-Fc fusion protein in the formulation is at or about 80 mg. In some embodiments, the concentration of the TACI-Fc fusion protein is between about 50 mg/mL and about 200 mg/mL. In some embodiments, the concentration of the TACI-Fc fusion protein is at or about 100 mg/mL.

In some embodiments, the TACI-Fc fusion protein is administered intravenously. In some embodiments, the TACI-Fc fusion protein is administered subcutaneously.

In some embodiments, a B cell immune response or activity is reduced in the subject. In some embodiments, the numbers of mature and total circulating B cells is reduced in the subject. In some embodiments, circulating serum immunoglobulins are reduced in the subject. In some embodiments, one or more of B cell maturation, differentiation, and/or proliferation is reduced or inhibited. In some embodiments, B cell immune response or activity is assessed by measuring immunoglobulins secreted by B cells (e.g., IgA, IgG, IgM, and/or IgE). In some embodiments, B cell maturation, differentiation, and/or proliferation is assessed by immunophenotyping B cells. In some embodiments, B cell maturation, differentiation, and/or proliferation is assessed by measuring immunoglobulins secreted by B cells (e.g., IgA, IgG, IgM, and/or IgE).

In some embodiments, circulating levels of an APRIL or BAFF protein are reduced in the subject. In some embodiments, circulating levels of free APRIL (i.e., unbound APRIL) are reduced in the subject. In some embodiments, circulating levels of free BAFF (i.e., unbound BAFF) are reduced in the subject. In some embodiments, the APRIL or BAFF protein is an APRIL homotrimer, BAFF homotrimer, APRIL/BAFF heterotrimer, or BAFF 60mer. In some embodiments, the subject is a human. In some embodiments, the subject is an adult subject, such as 18 years of age or older, for example 18-65 years of age.

### 5. Neurologic Disease

In some embodiments, the provided TACI-Fc fusion proteins can be used for treating neurologic disease like encephalitis. In some embodiments, encephalitis comprises autoimmune encephalitis or limbic encephalitis. In other embodiments, the neurologic disease comprises chronic inflammatory demyelinating polyneuropathy (CIDP), acute inflammatory demyelinating polyneuropathy (AIDP), multiple sclerosis, myasthenia gravis, or multifocal motor neuropathy (MMN). In other embodiments, the neurologic disease comprises Neuromyelitis optica spectrum disorder (NMSOD).

In some aspects, provided herein is a method of treating encephalitis comprising administering the TACI-Fc fusion proteins provided herein. In some embodiments, the method comprises: a) selecting a subject for administration of a TACI-Fc fusion protein that has been diagnosed with Encephalitis; and b) administering to the selected subject the TACI-Fc fusion protein in accord with provided methods and doses (e.g. described in Section VI.B), wherein: the TACI-Fc fusion protein is a homodimer of two polypeptides of the formula TACI-linker-Fc, wherein TACI is a variant TACI polypeptide such as any as provided herein. In some embodiments, the variant TACI polypeptide comprises the amino acid substitutions K77E, F78Y and Y102D in the amino acid sequence set forth in SEQ ID NO:13. In some embodiments, the TACI-Fc fusion protein is administered subcutaneously at a dose of from at or about 80 mg to at or about 480 mg once every four weeks.

In some embodiments, the TACI-Fc fusion protein is administered at a dose that includes any dose as described in Section VI.B. In some embodiments, the dose is from at or about 80 mg to at or about 240 mg. In some embodiments, the dose is at or about 80 mg. In some embodiments, the dose is at or about 240 mg.

In some embodiments, the TACI-Fc fusion protein is administered once every four weeks (Q4W). In some embodiments, the dose is from at or about 80 mg to at or about 240 mg Q4W. In some embodiments, the dose is at or about 80 mg Q4W. In some embodiments, the dose is at or about 240 mg Q4W. In some embodiments, the dose is from at or about 80 mg to at or about 960 mg Q4W. In some embodiments, the dose is at or about 960 mg Q4W.

In some embodiments, the encephalitis is autoimmune encephalitis. In some embodiments, the encephalitis is limbic encephalitis.

In some embodiments, the TACI-Fc fusion protein is administered to the subject at the dosing regimen for at least 12-weeks, 16-weeks, 20-weeks, 24-weeks, 28-weeks, 32-weeks, 36-weeks, 40-weeks, 44-weeks, 48-weeks, 52-weeks, 56 weeks, 60 weeks, 64 weeks, 68 weeks, 72 weeks. In some embodiments, the TACI-Fc fusion protein is administered to the subject at the dosing regimen for about 2-weeks to about 2 years, such as from 12-weeks, 16-weeks, 20-weeks, 24-weeks, 28-weeks, 32-weeks, 36-weeks, 40-weeks, 44-weeks, 48-weeks, 52-weeks, or any value time between any of the foregoing. In some embodiments, the period of time is for at or about 28-weeks. In some embodiments, the administration regimen continues, such as at the direction of a medical provider or clinician, for example until disease progresses.

In some embodiments, the TACI-Fc fusion protein is administered to the subject Q4W for between 12 weeks and 72 weeks. In some embodiments, the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

In some embodiments, the TACI-Fc fusion protein is administered to the subject Q4W for 12 weeks. In some embodiments, the TACI-Fc fusion protein is administered to the subject Q4W for 16 weeks. In some embodiments, the TACI-Fc fusion protein is administered to the subject Q4W for 24 weeks. In some embodiments, the TACI-Fc fusion protein is administered to the subject Q4W for 48 weeks.

In some embodiments, during the course of the treatment, the subject may be monitored for one or more endpoints of the disease to assess efficacy and/or one or more for an adverse event. In some embodiments, efficacy is monitored by modified Rankin Scale (mRS). In some embodiments, efficacy is measured by clinical assessment scale of autoimmune encephalitis (CASE).

In some embodiments, the variant TACI polypeptide is set forth in SEQ ID NO:26.

In some embodiments, the linker is a GS linker of between 5 and 20 amino acids in length. In some embodiments, the linker is selected from GSGGS (SEQ ID NO: 76), GGGGS (G4S; SEQ ID NO: 77), GSGGGGS (SEQ ID NO: 74), GGGGSGGGGS (2×GGGGS; SEQ ID NO: 78), GGGGSGGGGSGGGGS (3×GGGGS; SEQ ID NO: 79), GGGGSGGGGSGGGGSGGGGS (4×GGGGS, SEQ ID NO:84), GGGGSGGGGSGGGGSGGGGSGGGGS (5×GGGGS, SEQ ID NO: 91), GGGGSSA (SEQ ID NO: 80), or GSGGGGSGGGGS (SEQ ID NO:194) or combinations thereof. In some embodiments, the linker is set forth in SEQ ID NO: 74.

In some embodiments, the Fc is an IgG1 Fc domain. In some embodiments, the Fc is a variant IgG1 Fc that exhibits reduced binding affinity to an Fc receptor and/or reduced effector function as compared to a wild-type IgG1 Fc domain. In some embodiments, the variant IgG1 Fc domain comprises one or more amino acid substitutions selected from L234A, L234V, L235A, L235E, G237A, S267K, R292C, N297G, and V302C, by EU numbering. In some embodiments, the variant IgG1 Fc comprises the amino acid substitutions L234A, L235E, and G237A by EU numbering. In some embodiments, the Fc comprises the amino acid substitution C220S, wherein the residues are numbered according to the EU index of Kabat. In some embodiments, the Fc lacks the hinge sequence EPKSS or EPKSC. In some embodiments, the Fc region comprises K447del, wherein the residue is numbered according to the EU index of Kabat.

In some embodiments, the Fc comprises the amino acid sequence set forth in SEQ ID NO:73. In some embodiments, the TACI-Fc fusion protein is set forth in SEQ ID NO: 167.

In some embodiments, the Fc comprises the amino acid sequence set forth in SEQ ID NO:81. In some embodiments, the TACI-Fc fusion protein is set forth in SEQ ID NO: 168.

In some embodiments, the TACI-Fc fusion protein is provided in a formulation comprising an acetic acid buffer having a pH of from about 4.0 to about 6.0, proline at a concentration of from at or about 1% to about 10%, and a surfactant at a concentration of from about 0.005 to about 0.05% (w/v). In some embodiments, the formulation has a pH of about 5.2. In some embodiments, the acetic acid buffer comprises a concentration of acetate of from at or about 5 mM to at or about 15 mM. In some embodiments, the acetic acid buffer comprises a concentration of acetate of at or about 10 mM. In some embodiments, the proline is at a concentration of about 2% to about 5%. In some embodiments, the proline is at a concentration of at or about 3%. In some embodiments, the surfactant is at a concentration of from about 0.01 to about 0.025% (w/v), such as at or about 0.015% (w/v). In some embodiments, the surfactant is polysorbate 80. In some embodiments, the amount of TACI-Fc fusion protein in the formulation is from about 50 mg to about 100 mg. In some embodiments, the amount of TACI-Fc fusion protein in the formulation is at or about 80 mg. In some embodiments, the concentration of the TACI-Fc fusion protein is between about 50 mg/mL and about 200 mg/mL. In some embodiments, the concentration of the TACI-Fc fusion protein is at or about 100 mg/mL.

In some embodiments, the TACI-Fc fusion protein is administered intravenously. In some embodiments, the TACI-Fc fusion protein is administered subcutaneously.

In some embodiments, a B cell immune response or activity is reduced in the subject. In some embodiments, the numbers of mature and total circulating B cells is reduced in the subject. In some embodiments, circulating serum immunoglobulins are reduced in the subject. In some embodiments, one or more of B cell maturation, differentiation, and/or proliferation is reduced or inhibited. In some embodiments, B cell immune response or activity is assessed by measuring immunoglobulins secreted by B cells (e.g., IgA, IgG, IgM, and/or IgE). In some embodiments, B cell maturation, differentiation, and/or proliferation is assessed by immunophenotyping B cells. In some embodiments, B cell maturation, differentiation, and/or proliferation is assessed by measuring immunoglobulins secreted by B cells (e.g., IgA, IgG, IgM, and/or IgE).

In some embodiments, circulating levels of an APRIL or BAFF protein are reduced in the subject. In some embodiments, circulating levels of free APRIL (i.e., unbound APRIL) are reduced in the subject. In some embodiments, circulating levels of free BAFF (i.e., unbound BAFF) are reduced in the subject. In some embodiments, the APRIL or BAFF protein is an APRIL homotrimer, BAFF homotrimer, APRIL/BAFF heterotrimer, or BAFF 60mer. In some embodiments, the subject is a human. In some embodiments, the subject is an adult subject, such as 18 years of age or older, for example 18-65 years of age.

### VII. ARTICLES OF MANUFACTURE AND KITS

Also provided herein are articles of manufacture that comprise the pharmaceutical compositions described herein in suitable packaging. Suitable packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

Further provided are kits comprising the pharmaceutical compositions (or articles of manufacture) described herein, which may further comprise instruction(s) on methods of using the composition, such as uses described herein. The kits described herein may also include other materials desirable from a commercial and user standpoint, including other buffers, diluents, filters, needles, syringes, and package inserts with instructions for performing any methods described herein.

### VIII. EXEMPLARY EMBODIMENTS

Among the provided embodiments are:

1. A method of treating an autoantibody-related disease or disorder in a subject, the method comprising administering to the subject a TACI-Fc fusion protein that is a homodimer of two polypeptides of the formula TACI-linker-Fc, wherein TACI is a variant TACI polypeptide comprising one or more amino acid substitutions selected from the group consisting of K77E, F78Y and Y102D in the amino acid sequence set forth in SEQ ID NO:13, and wherein the TACI-Fc fusion protein is administered subcutaneously at a dose of from at or about 80 mg to at or about 480 mg once every four weeks (Q4W).

2. A method of treating an autoantibody-related disease or disorder in a subject, the method comprising administering to the subject a TACI-Fc fusion protein that is a homodimer of two polypeptides of the formula TACI-linker-Fc, wherein TACI is a variant TACI polypeptide comprising one or more amino acid substitutions selected from the group consisting of K77E, F78Y and Y102D in the amino acid sequence set forth in SEQ ID NO:13, and wherein the TACI-Fc fusion protein is administered subcutaneously at a dose of from at or about 24 mg to at or about 480 mg once every four weeks (Q4W), once every eight weeks (Q8W), or once every twelve weeks (Q12W).

3. A method of treating an autoantibody-related disease or disorder in a subject, the method comprising administering to the subject a TACI-Fc fusion protein that is a homodimer of two polypeptides of the formula TACI-linker-Fc, wherein TACI is a variant TACI polypeptide comprising one or more amino acid substitutions selected from the group consisting of K77E, F78Y and Y102D in the amino acid sequence set forth in SEQ ID NO:13, and wherein the TACI-Fc fusion protein is administered subcutaneously at a dose of from at or about 24 mg to at or about 960 mg once every four weeks (Q4W), once every eight weeks (Q8W), or once every twelve weeks (Q12W).

4. The method of embodiment 1, wherein the variant TACI polypeptide comprises the amino acid substitutions K77E, F78Y and Y102D.

5. The method of any of embodiments 1-3, wherein the dose is from at or about 80 mg to at or about 240 mg Q4W.

6. The method of any of embodiments 1-5, wherein the dose is at or about 80 mg Q4W.

7. The method of any of embodiments 1-5, wherein the dose is at or about 240 mg Q4W.

8. The method of embodiment 2, wherein the dose is from at or about 24 mg to at or about 240 mg once every four weeks (Q4W), once every eight weeks (Q8W), or once every twelve weeks (Q12W).

9. The method of embodiment 2, wherein the dose is from at or about 24 mg to at or about 960 mg once every four weeks (Q4W), once every eight weeks (Q8W), or once every twelve weeks (Q12W).

10. The method of embodiment 2 or embodiment 3, wherein:
(i) the dose is at or about 24 mg Q4W;
(ii) the dose is at or about 24 mg Q8W;
(iii) the dose is at or about 24 mg Q12W;
(iv) the dose is at or about 80 mg Q8W;
(v) the dose is at or about 80 mg Q12W;
(vi) the dose is at or about 240 mg Q8W;
(vii) the dose is at or about 240 mg Q12W
(viii) the dose is at or about 960 mg Q8W; or
(ix) the dose is at or about 960 mg Q12W.

11. The method of any of embodiments 1-10, wherein the autoantibody-related disease or disorder is selected from the group consisting of a rheumatic disease or disorder, a renal (kidney) disease or disorder, a hematologic disease or disorder, a dermatologic disease or disorder, or a neurologic disease or disorder.

12. The method of any of embodiments 1-11, wherein the autoantibody-related disease or disorder is a rheumatic disease or disorder.

13. The method of any of embodiments 1-12, wherein the autoantibody-related disease or disorder is Sjogren's.

14. The method of any of embodiments 1-12, wherein the autoantibody-related disease or disorder is Systemic lupus erythematosus (SLE)

15. The method of any of embodiments 1-14, wherein the TACI-Fc fusion protein reduces the risk of the subject developing hypogammaglobulinemia or severe hypogammaglobulinemia.

16. The method of embodiment 15, wherein hypogammaglobulinemia is characterized by circulating IgG≤7 g/L.

17. The method of embodiment 15, wherein severe hypogammaglobulinemia is characterized by circulating IgG≤3 g/L.

18. The method of any of embodiments 1-17, wherein the TACI-Fc fusion protein reduces the amount of circulating immunoglobulin G (IgG)

19. The method of embodiment 18, wherein circulating IgG is reduced by about 35% from the subject's baseline

20. A method of treating Systemic lupus erythematosus (SLE), the method comprising:
a) selecting a subject for administration of a TACI-Fc fusion protein that has been diagnosed with SLE; and
b) administering to the selected subject the TACI-Fc fusion protein, wherein:
   the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
   the TACI-Fc fusion protein is administered subcutaneously at a dose of from at or about 80 mg to at or about 480 mg once every four weeks.

21. A method of treating Systemic lupus erythematosus (SLE), the method comprising:
a) selecting a subject for administration of a TACI-Fc fusion protein that has been diagnosed with SLE; and
b) administering to the selected subject the TACI-Fc fusion protein, wherein:
   the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
   the TACI-Fc fusion protein is administered subcutaneously at a dose of from at or about 80 mg to at or about 960 mg once every four weeks (Q4W), once every eight weeks (Q8W), or once every twelve weeks (Q12W).

22. The method of embodiment 20, wherein the dose is from at or about 80 mg to at or about 240 mg Q4W.

23. The method of embodiment 20 or embodiment 22, wherein the dose is at or about 80 mg Q4W.

24. The method of embodiment 20 or embodiment 22, wherein the dose is at or about 240 mg Q4W.

25. The method of embodiment 21, wherein the dose is at or about 960 Q12W.

26. The method of any of embodiments 14-24, wherein the systemic lupus erythematosus is mild to moderate systemic lupus erythematosus or moderate to severe systemic lupus erythematosus.

27. The method of any of embodiments 14-26, wherein the subject is selected for treatment if at the time of screening the subject has active SLE for 6 months.

28. The method of any of embodiments 14-27, wherein the subject is selected for treatment if at the time of screening the SLE is characterized by one or more of the following:
(i) a hybrid SELENA-SLEDAI score 8 or a hybrid SELENA-SLEDAI 6 if there is high anti-dsDNA or low complement (C) levels;
(ii) ≤6 g/g urine total protein to creatinine ratio (proteinuria);
(iii) A grade in the BILAG score in 1 organs;
(iv) B grade in the BILAG score in 2 organs; and
(v) Physicians Global Assessment (PGA) score 1.0.

29. The method of any of embodiments 14-28, wherein the subject is receiving standard therapy for treating the SLE.

30. The method of embodiment 14-29, wherein the subject is selected for treatment if the at the time of screening or the time of administering the TACI-Fc fusion protein, the subject is receiving a stable standard treatment regimen characterized by the stable use of a standard therapy for treating the SLE, optionally wherein the stable use is stable use of the standard therapy for at least 30 days.

31. The method of any of embodiments 14-29, wherein the TACI-Fc fusion protein is administered to the subject in combination with a standard therapy for treating the SLE.

32. The method of any of embodiments 29-31, wherein the standard therapy comprises one of more of a corticosteroid, antimalarial (e.g. hydroxychloroquine), a non-steroidal anti-inflammatory drug (NSAID), or an immunosuppressant or immunomodulator, or any combination thereof.

33. The method of embodiment 32, wherein the immunosuppressant or immunomodulator is selected from the group consisting of including azathioprine, mycophenolate (e.g. mycophenolate mofetil or sodium mycophenolate), cyclophosphamide, methotrexate, leflunomide, tacrolimus, cyclosporine and combinations of any of the foregoing.

34. The method of any of embodiments 29-33, wherein the standard therapy comprises a corticosteroid and administration of the corticosteroid is tapered after administering the

TACI-Fc fusion protein.

35. The method of any of embodiments 14-34, wherein the subject is selected for treatment if at the time of screening the subject is characterized by one or more of the following:
(i) severe lupus nephritis, optionally defined as urine protein >6 g/24 hours or serum creatinine>2.5 mg/dL or 221 μmol/L;
(ii) required hemodialysis;
(iii) received high-dose corticosteroids for ≥14 days in the last 2 months, optionally wherein the high-dose corticosteroid is treatment with prednisone>100 mg/day or equivalent; and
(iv) central nervous system disease caused by SLE or not caused by SLE in the last 2 months; optionally wherein the central nervous system disease is epilepsy, psychosis, organic brain syndrome, cerebrovascular accident, encephalitis, or central nervous system vasculitis.

36. The method of any of embodiments 1-11, wherein the autoantibody-related disease or disorder is a renal (kidney) disease or disorder.

37. The method of any of embodiments 1-11 and 36, wherein the autoantibody-related disease or disorder is a Glomerulonephritis.

38. A method of treating a Glomerulonephritis, the method comprising:
a) selecting a subject for administration of a TACI-Fc fusion protein that has been diagnosed with glomerulonephritis; and
b) administering to the selected subject the TACI-Fc fusion protein, wherein:
   the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
   the TACI-Fc fusion protein is administered subcutaneously at a dose of from at or about 80 mg to at or about 480 mg once every four weeks (Q4W).

39. A method of treating a Glomerulonephritis, the method comprising:
a) selecting a subject for administration of a TACI-Fc fusion protein that has been diagnosed with glomerulonephritis; and
b) administering to the selected subject the TACI-Fc fusion protein, wherein:
   the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
the TACI-Fc fusion protein is administered subcutaneously at a dose of from at or about 80 mg to at or about 960 mg once every four weeks (Q4W), once every eight weeks (Q4W), or once every twelve weeks (Q12W).

40. The method of embodiment 38, wherein the dose is from at or about 80 mg to at or about 240 mg Q4W.

41. The method of embodiment 38 or embodiment 40, wherein the dose is at or about 80 mg Q4W.

42. The method of embodiment 38 or embodiment 40, wherein the dose is at or about 240 mg Q4W.

43. The method of embodiment 39, wherein the dose is at or about 960 mg Q12W.

44. The method of any of embodiments 38-43, wherein the subject is selected for treatment if at the time of screening the subject has active Glomerulonephritis.

45. The method of any of embodiments 38-44, wherein the Glomerulonephritis is selected from the group consisting of IgA Nephropathy, Lupus Nephritis and Primary Membranous Nephropathy.

46. The method of any of embodiments 38-45, wherein the Glomerulonephritis is IgA Nephropathy and the subject is selected for treatment if at the time of screening the subject is characterized by one or both of the following:
(i) the subject was diagnosed with IgA Nephropathy ≤5 years prior to the screening; and
(ii) ≥0.75 g/g urine total protein to creatinine (proteinuria).

47. The method of any of embodiments 38-45, wherein the Glomerulonephritis is IgA Nephropathy and the subject is selected for treatment if at the time of screening the subject is characterized by one or more of the following:
(i) the subject was diagnosed with IgA Nephropathy ≤5 years prior to the screening;
(ii) ≥0.75 g/g urine total protein to creatinine (proteinuria); and
(iii) elevated galactose deficient IgAQ1 (Gd-IgAQ1)

48. The method of embodiment 47, wherein the TACI-Fc fusion protein reduces Gd-IgA1.

49. The method of embodiment 48, wherein Gd-IgA1 is reduced by more than 50%

50. The method of any of embodiments 38-45, wherein the Glomerulonephritis is Lupus Nephritis and the Lupus Nephritis is Class III (active focal), Class IV (diffuse) or Class V (lupus membranous nephropathy).

51. The method of any of embodiments 38-45 and 50, wherein the Glomerulonephritis is Lupus Nephritis and the subject is selected for treatment if at the time of screening the subject is characterized by one or more of the following:
(i) the subject was diagnosed with Lupus Nephritis Class II-V≤3 years prior to the screening;
(ii) ≥1 g/g urine total protein to creatinine ratio (proteinuria);
(iii) active urinary sediment;
(iv) positive anti-dsDNA and antinuclear antibodies (ANA), optionally wherein positive anti-dsDNA is a titer of ≥30 IU/mL and positive ANA is a titer of ≥1:80;
(v) stable standard treatment regimen characterized by the stable use of a standard therapy for treating the SLE, optionally wherein the stable use is stable use of the standard therapy for at least 30 days; and
(v) received stable background immunosuppression, optionally wherein the stable background immunosuppression is a stable dose of MMF of 1 g/day, with or without corticosteroids, for at least 8 weeks prior to the time of screening or the time of administering the TACI-Fc fusion protein.

52. The method of any of embodiments 38-45 and 50, wherein the Glomerulonephritis is Lupus Nephritis and the subject is selected for treatment if at the time of screening the subject is characterized by one or more of the following:
(i) the subject was diagnosed with Lupus Nephritis Class II-V≤3 years prior to the screening;
(ii) ≥1 g/g urine total protein to creatinine ratio (proteinuria);
(iii) active urinary sediment;
(iv) positive antinuclear antibody (ANA) and/or anti-dsDNA antibody, optionally wherein positive anti-dsDNA is a titer of ≥30 IU/mL and positive ANA is a titer of ≥1:80;
(v) stable standard treatment regimen characterized by the stable use of a standard therapy for treating the SLE, optionally wherein the stable use is stable use of the standard therapy for at least 30 days; and
(v) received stable background immunosuppression, optionally wherein the stable background immunosuppression is a stable dose of MMF of ≥1 g/day, with or without corticosteroids, for at least 8 weeks prior to the time of screening or the time of administering the TACI-Fc fusion protein.
   wherein the stable background immunosuppression is azathioprine, mycophenolic acid (MPA), and/or calcineurin inhibitors (CnIs).

53. The method of any of embodiments 38-45, wherein the Glomerulonephritis is primary Membranous Nephropathy.

54. The method of any of embodiments 38-45 and 53, wherein the Glomerulonephritis is primary Membranous Nephropathy (pMN) and the subject is selected for treatment if at the time of screening the subject is characterized by one or more of the following:
(i) the subject was diagnosed with pMN≤5 years prior to the screening;
(ii) ≥3.5 g/g urine total protein to creatinine ratio (proteinuria); and/or
(iii) positive anti-PLA2R1 or positive anti-THSD7A antibodies.

55. The method of any of embodiments 38-54, wherein the subject is selected for treatment if at the time of screening or the time of administering the TACI-Fc fusion protein the subject has received therapy with an Angiotensin-converting enzyme (ACE) inhibitor and/or angiotensin II receptor blocker (ARB), optionally wherein the subject has received a maximally recommended dose of the ACE inhibitor or ARB therapy.

56. The method of any of embodiments 38-55, wherein the subject is selected for treatment if at the time of screening or at the time of administering the TACI-Fc fusion protein the subject has a stable blood pressure.

57. The method of any of embodiments 1-11, wherein the autoantibody-related disease or disorder is a hematological disease or disorder.

58. The method of any of embodiments 1-11 and 57, wherein the autoantibody-related disease or disorder is an autoimmune cytopenia.

59. A method of treating an autoimmune cytopenia, the method comprising:
a) selecting a subject for administration of a TACI-Fc fusion protein that has been diagnosed with an autoimmune cytopenia; and
b) administering to the selected subject the TACI-Fc fusion protein, wherein:
   the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
   the TACI-Fc fusion protein is administered subcutaneously at a dose of from at or about 80 mg to at or about 480 mg once every four weeks (Q4W).

60. A method of treating an autoimmune cytopenia, the method comprising:
a) selecting a subject for administration of a TACI-Fc fusion protein that has been diagnosed with an autoimmune cytopenia; and
b) administering to the selected subject the TACI-Fc fusion protein, wherein:
   the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
   the TACI-Fc fusion protein is administered subcutaneously at a dose of from at or about 80 mg to at or about 960 mg once every four weeks (Q4W), once every eight weeks (Q8W), or once every twelve weeks (Q12W).

61. The method of embodiment 59, wherein the dose is from at or about 80 mg to at or about 240 mg Q4W.

62. The method of embodiment 59 or embodiment 61, wherein the dose is at or about 80 mg Q4W.

63. The method of embodiment 59 or embodiment 61, wherein the dose is at or about 240 mg Q4W.

64. The method of embodiment 60, wherein the dose is at or about 960 mg Q12W.

65. The method of any of embodiments 58-64, wherein the subject is selected for treatment if at the time of screening the subject has active cytopenia.

66. The method of any of embodiments 58-65, wherein the autoimmune cytopenia is selected from the group consisting of Immune Thrombocytopenia (ITP) and Autoimmune Hemolytic Anemia (AIHA).

67. The method of any of embodiments 58-66, wherein the autoimmune cytopenia is ITP and the subject is selected for treatment if at the time of screening the subject is characterized by one or both of the following:
(i) the subject was diagnosed with ITP≥3 months prior to the screening; and
(ii) sustained platelet count <30,000/μL.

68. The method of embodiment 67, wherein the subject has received ≥2 prior treatments for treating the ITP.

69. The method of embodiment 67 or embodiment 68, wherein the subject has received ≥4 prior treatments for treating the ITP.

70. The method of any of embodiments 58-66, wherein the autoimmune cytopenia is an AIHA and the AIHA is warm AIHA (wAIHA) or cold AIHA (cold agglutinin disease, CAD).

71. The method of any of embodiments 58-66 and 70, wherein the autoimmune cytopenia is wAIHA or CAD and the subject is selected for treatment if at the time of screening the subject is characterized by one or both of the following:
(i) the subject was diagnosed with wAIHA or CAD≥3 months prior to the screening; and
(ii) sustained hemoglobin (Hb)<9 g/dL.

72. The method of embodiment 71, wherein the subject has received ≥1 prior treatments for treating the AIHA.

73. The method of embodiment 71 or embodiment 72, wherein the subject has received ≥2 treatments for treating the AIHA.

74. The method of embodiment 71, wherein the autoimmune cytopenia is wAIHA.

75. The method of embodiment 71, wherein the autoimmune cytopenia is CAD.

76. The method of embodiment 58, wherein the subject is selected for treatment if at the time of screening or the time of administering the TACI-Fc fusion protein, the subject is receiving a stable immunosuppression.

77. The method of embodiment 76, wherein the TACI-Fc fusion protein is administered to the subject in combination with concurrent administration of the stable immunosuppression.

78. The method of embodiment 76 or embodiment 77, wherein:
the stable immunosuppression comprises a stable dose of a steroid, optionally a corticosteroid, for at least two weeks prior to the time of screening or the time of administering the TACI-Fc fusion protein; and/or
the stable immunosuppression comprises a stable dose of azathioprine, MMF, or cyclosporine for at least four weeks prior to the time of screening or the time of administering the TACI-Fc fusion protein.

79. The method of any of embodiments 58-78, wherein the subject is not characterized by having a secondary cytopenia (e.g. systemic autoimmune disease or malignancy) or Evans syndrome.

80. The method of any of embodiments 1-11, wherein the autoantibody-related disease or disorder is a dermatologic disease or disorder.

81. The method of any of embodiments 1-11 and 80, wherein the autoantibody-related disease or disorder is an autoimmune bullous dermatosis.

82. A method of treating an autoimmune bullous (blistering) dermatosis, the method comprising:
a) selecting a subject for administration of a TACI-Fc fusion protein that has been diagnosed with an autoimmune bullous (blistering) dermatosis; and
b) administering to the selected subject the TACI-Fc fusion protein, wherein:
   the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
   the TACI-Fc fusion protein is administered subcutaneously at a dose of from at or about 80 mg to at or about 480 mg once every four weeks (Q4W).

83. A method of treating an autoimmune bullous (blistering) dermatosis, the method comprising:
a) selecting a subject for administration of a TACI-Fc fusion protein that has been diagnosed with an autoimmune bullous (blistering) dermatosis; and
b) administering to the selected subject the TACI-Fc fusion protein, wherein:
   the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
   the TACI-Fc fusion protein is administered subcutaneously at a dose of from at or about 80 mg to at or about 960 mg once every four weeks (Q4W), once every eight weeks (Q8W), or once every twelve weeks (Q12W).

84. The method of embodiment 82, wherein the dose is from at or about 80 mg to at or about 240 mg Q4W.

85. The method of embodiment 82 or embodiment 84, wherein the dose is at or about 80 mg Q4W.

86. The method of embodiment 82 or embodiment 84, wherein the dose is at or about 240 mg Q4W.

87. The method of embodiment 83, wherein the dose is at or about 960 mg Q12W.

88. The method of any of embodiments 81-86, wherein the subject is selected for treatment if at the time of screening the subject has active blistering disease.

89. The method of any of embodiments 81-88, wherein the autoimmune bullous (blistering) dermatosis is selected from the group consisting of Pemphigus vulgaris, Pemphigus foliaceus or Bullous Pemphigoid.

90. The method of any of embodiments 81-89, wherein the autoimmune bullous (blistering) dermatosis is Pemphigus vulgaris or Pemphigus foliaceus and the subject is selected for treatment if at the time of screening the subject is characterized by one or both of the following:
(i) a Pemphigus Disease Area Index (PDAI)≥15; and
(ii) positive anti-Dsg1 or positive anti-Dsg3 antibodies.

91. The method of embodiment 90, wherein the autoimmune bullous (blistering) dermatosis is Pemphigus vulgaris.

92. The method of embodiment 91, wherein the autoimmune bullous (blistering) dermatosis is Pemphigus foliaceus.

93. The method of any of embodiments 81-90, wherein the autoimmune bullous (blistering) dermatosis is Pemphigoid and the subject is selected for treatment if at the time of screening the subject is characterized by one or both of the following:
(i) IgA antibodies; and
(ii) positive anti-Bp180 or positive anti-Bp230 antibodies.

94. The method of embodiment 81-93, wherein the subject is selected for treatment if at the time of screening or the time of administering the TACI-Fc fusion protein, the subject is receiving a stable immunosuppression.

95. The method of embodiment 94, wherein the TACI-Fc fusion protein is administered to the subject in combination with concurrent administration of the stable immunosuppression.

96. The method of embodiment 94 or embodiment 95, wherein:
the stable immunosuppression comprises a stable dose of a steroid, optionally a corticosteroid, for at least two weeks prior to the time of screening or the time of administering the TACI-Fc fusion protein; and/or
the stable immunosuppression comprises a stable dose of azathioprine, MMF, or cyclosporine for at least four weeks prior to the time of screening or the time of administering the TACI-Fc fusion protein.

97. The method of any of embodiments 81-96, wherein the subject is not characterized by having a secondary disease (e.g. paraneoplastic).

98. The method of any of embodiments 1-11, wherein the autoantibody-related disease or disorder is a neurologic disease or disorder.

99. The method of any of embodiments 1-11 and 98, wherein the autoantibody-related disease or disorder is Encephalitis.

100. A method of treating Encephalitis, the method comprising:
a) selecting a subject for administration of a TACI-Fc fusion protein that has been diagnosed with Encephalitis; and
b) administering to the selected subject the TACI-Fc fusion protein, wherein:
   the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
   the TACI-Fc fusion protein is administered subcutaneously at a dose of from at or about 80 mg to at or about 480 mg once every four weeks (Q4W).

101. A method of treating Encephalitis, the method comprising:
a) selecting a subject for administration of a TACI-Fc fusion protein that has been diagnosed with Encephalitis; and
b) administering to the selected subject the TACI-Fc fusion protein, wherein:
   the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
   the TACI-Fc fusion protein is administered intravenously at a dose of from at or about 80 mg to at or about 480 mg once every four weeks (Q4W).

102. A method of treating Encephalitis, the method comprising:
a) selecting a subject for administration of a TACI-Fc fusion protein that has been diagnosed with Encephalitis; and
b) administering to the selected subject the TACI-Fc fusion protein, wherein:
   the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
   the TACI-Fc fusion protein is administered intravenously at a dose of from at or about 80 mg to at or about 960 mg once every four weeks (Q4W), once every eight weeks (Q8W), or once every twelve weeks (Q12W).

103. The method of embodiment 100, wherein the dose is from at or about 80 mg to at or about 240 mg Q4W.

104. The method of embodiment 100 or embodiment 103, wherein the dose is at or about 80 mg Q4W.

105. The method of embodiment 100 or embodiment 103, wherein the dose is at or about 240 mg Q4W.

106. The method of embodiment 102, wherein the dose is at or about 960 mg Q12W.

107. The method of any of embodiments 99-105, wherein the Encephalitis is autoimmune encephalitis.

108. The method of any of embodiments 99-107, wherein the Encephalitis is Limbic encephalitis.

109. The method of any of embodiments 1-108, wherein the TACI-Fc fusion protein is administered to the subject Q4W for between 12 weeks and 72 weeks.

110. The method of any of embodiments 1-109, wherein the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

111. The method of any of embodiments 1-110, wherein the TACI-Fc fusion protein is administered to the subject Q4W for 12 weeks.

112. The method of any of embodiments 1-110, wherein the TACI-Fc fusion protein is administered to the subject Q4W for 16 weeks.

113. The method of any of embodiments 1-110, wherein the TACI-Fc fusion protein is administered to the subject Q4W for 24 weeks.

114. The method of any of embodiments 1-110, wherein the TACI-Fc fusion protein is administered to the subject Q4W for 48 weeks.

115. The method of any of embodiments 1-114, wherein the variant TACI polypeptide is set forth in SEQ ID NO:26.

116. The method of any of embodiments 1-114, wherein the linker is a GS linker of between 5 and 20 amino acids in length.

117. The method of any of embodiments 1-116, wherein the linker is selected from GSGGS (SEQ ID NO: 76), GGGGS (G4S; SEQ ID NO: 77), GSGGGGS (SEQ ID NO: 74), GGGGSGGGGS (2×GGGGS; SEQ ID NO: 78), GGGGSGGGGSGGGGS (3×GGGGS; SEQ ID NO: 79), GGGGSGGGGSGGGGSGGGGS (4×GGGGS, SEQ ID NO:84), GGGGSGGGGSGGGGSGGGGSGGGGS (5×GGGGS, SEQ ID NO: 91), GGGGSSA (SEQ ID NO: 80), or GSGGGGSGGGGS (SEQ ID NO:194) or combinations thereof.

118. The method of any of embodiments 1-117, wherein the linker is set forth in SEQ ID NO: 74.

119. The method of any of embodiments 1-118, wherein the Fc is an IgG1 Fc domain.

120. The method of any of embodiments 1-119, wherein the Fc is a variant IgG1 Fc that exhibits reduced binding affinity to an Fc receptor and/or reduced effector function as compared to a wild-type IgG1 Fc domain.

121. The method of embodiment 120, wherein the variant IgG1 Fc domain comprises one or more amino acid substitutions selected from L234A, L234V, L235A, L235E, G237A, S267K, R292C, N297G, and V302C, by EU numbering.

122. The method of embodiment 120 or embodiment 121, wherein the variant IgG1 Fc comprises the amino acid substitutions L234A, L235E, and G237A by EU numbering.

123. The method of any of embodiments 119-122, wherein the Fc comprises the amino acid substitution C220S, wherein the residues are numbered according to the EU index of Kabat.

124. The method of any of embodiments 119-123, wherein the Fc lacks the hinge sequence EPKSS or EPKSC

125. The method of any of embodiments 119-124, wherein the Fc region comprises K447del, wherein the residue is numbered according to the EU index of Kabat.

126. The method of embodiment 1-123 and 125, wherein the Fc comprises the amino acid sequence set forth in SEQ ID NO:73.

127. The method of any of embodiments 1-123, 125 and 126, wherein the TACI-Fc fusion protein is set forth in SEQ ID NO: 167.

128. The method of embodiment 1-119, 123-127, wherein the Fc comprises the amino acid sequence set forth in SEQ ID NO:81.

129. The method of any of embodiments 1-119, 123-127, and 128, wherein the TACI-Fc fusion protein is set forth in SEQ ID NO: 168.

130. The method of any of embodiments 1-129, wherein the TACI-Fc fusion protein is provided in a formulation comprising an acetic acid buffer having a pH of from about 4.0 to about 6.0, proline at a concentration of from at or about 1% to about 10%, and a surfactant at a concentration of from about 0.005 to about 0.05% (w/v).

131. The method of embodiment 130, wherein the formulation has a pH of about 5.2.

132. The method of embodiment 130 or embodiment 131, wherein the acetic acid buffer comprises a concentration of acetate of from at or about 5 mM to at or about 15 mM.

133. The method of any of embodiments 130-132, wherein the acetic acid buffer comprises a concentration of acetate of at or about 10 mM.

134. The method of any of embodiments 130-133, wherein the proline is at a concentration of about 2% to about 5%.

135. The method of any of embodiments 130-133, wherein the proline is at a concentration of at or about 3%.

136. The method of any of embodiments 130-135, wherein the surfactant is at a concentration of from about 0.01 to about 0.025% (w/v), optionally at or about 0.015% (w/v).

137. The method of any of embodiments 130-136, wherein the surfactant is polysorbate 80.

138. The method of any of embodiments 130-137, wherein the amount of TACI-Fc fusion protein in the formulation is from about 50 mg to about 100 mg.

139. The method of any of embodiments 130-138, wherein the amount of TACI-Fc fusion protein in the formulation is at or about 80 mg.

140. The method of any of embodiments 130-139, wherein the concentration of the TACI-Fc fusion protein is between about 50 mg/mL and about 200 mg/mL.

141. The method of any of embodiments 130-136, wherein the concentration of the TACI-Fc fusion protein is at or about 100 mg/mL.

142. The method of any of embodiment 1-141, wherein a B cell immune response or activity is reduced in the subject.

143. The method of any of embodiment 1-142, wherein the numbers of mature and total circulating B cells is reduced in the subject.

144. The method of any of embodiments 1-143, wherein circulating serum immunoglobulins are reduced in the subject.

145. The method of any of embodiments 1-144, wherein one or more of B cell maturation, differentiation, and/or proliferation is reduced or inhibited.

146. The method of any of embodiments 1-145, wherein circulating levels of an APRIL or BAFF protein are reduced in the subject, optionally wherein the APRIL or BAFF protein is an APRIL homotrimer, BAFF homotrimer, APRIL/BAFF heterotrimer, or BAFF 60mer.

147. The method of any of embodiments 1-146, wherein the subject is a human.

148. The method of embodiment 147, wherein the subject is an adult subject, optionally 18 years of age or older, optionally 18-65 years of age.

149. A method of treating Cold Autoimmune Hemolytic Anemia (cAIHA), the method comprising
a) selecting a subject for administration of a TACI-Fc fusion protein that has been diagnosed with cAIHA; and
b) administering to the selected subject the TACI-Fc fusion protein.

150. A method of treating Warm Autoimmune Hemolytic Anemia (wAIHA), the method comprising
a) selecting a subject for administration of a TACI-Fc fusion protein that has been diagnosed with wAIHA; and
b) administering to the selected subject the TACI-Fc fusion protein.

151. A method of treating Pemphigus Foliaceus, the method comprising
a) selecting a subject for administration of a TACI-Fc fusion protein that has been diagnosed with Pemphigus foliaceus; and
b) administering to the selected subject the TACI-Fc fusion protein.

152. A method of treating Encephalitis, the method comprising
a) selecting a subject for administration of a TACI-Fc fusion protein that has been diagnosed with encephalitis; and
b) administering to the selected subject the TACI-Fc fusion protein.

153. The method of any of embodiments 149-152, wherein the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13.

154. The method of any of embodiments 149-153, wherein the variant TACI polypeptide is set forth in SEQ ID NO:13.

155. The method of any of embodiments 149-153, wherein the variant TACI polypeptide is set forth in SEQ ID NO:26.

156. The method of any of embodiments 149-153, wherein the TACI-Fc fusion protein is selected from the group consisting of Atacicept, Telitacicept, BION-1301, Sibeprenlimab, and Belimumab.

157. The method of any of embodiments 149-156, wherein the TACI-Fc fusion protein is provided in a formulation comprising an acetic acid buffer having a pH of from about 4.0 to about 6.0, proline at a concentration of from at or about 1% to about 10%, and a surfactant at a concentration of from about 0.005 to about 0.05% (w/v).

158. The method of embodiment 157, wherein the formulation has a pH of about 5.2.

159. The method of embodiment 157 or embodiment 158, wherein the acetic acid buffer comprises a concentration of acetate of from at or about 5 mM to at or about 15 mM.

160. The method of any of embodiments 157-159, wherein the acetic acid buffer comprises a concentration of acetate of at or about 10 mM.

161. The method of any of embodiments 157-160, wherein the proline is at a concentration of about 2% to about 5%.

162. The method of any of embodiments 157-161, wherein the proline is at a concentration of at or about 3%.

163. The method of any of embodiments 157-162, wherein the surfactant is at a concentration of from about 0.01 to about 0.025% (w/v), optionally at or about 0.015% (w/v).

164. The method of any of embodiments 157-163, wherein the surfactant is polysorbate 80.

165. The method of any of embodiments 157-164, wherein the TACI-Fc fusion protein is administered subcutaneously at a dose of from at or about 80 mg to at or about 960 mg once every four weeks (Q4W).

166. The method of any of embodiments 157-164, wherein the TACI-Fc fusion protein is administered intravenously at a dose of from at or about 80 mg to at or about 960 mg once every four weeks (Q4W).

167. The method of any of embodiments 150 and 153-166, wherein the subject is selected for administration of the TACI-Fc fusion protein if the subject has:
a) been diagnosed with wAIHA≥3 months prior to screening;
b) received ≥2 prior treatments;
c) sustained Hb≤9 g/dL;
d) sustained anemia on 2 occasions during screening;
e) symptomatic anemia; and/or
f) positive DAT for anti-IgG for more than 3 months.

168. The method of any of embodiments 149 and 153-166, wherein the subject is selected for administration of the TACI-Fc fusion protein if the subject has:
a) been diagnosed with wAIHA 3 months prior to screening;
b) received ≥2 prior treatments;
c) sustained Hb≤9 g/dL;
d) sustained anemia on 2 occasions during screening;
e) symptomatic anemia;
f) a positive polyspecific direct antiglobulin test (DAT);
g) a positive monospecific direct antiglobulin test (DAT);
h) a cold agglutinin titer ≥64;
i) chronic hemolysis for more than 3 months; and/or
j) an IgG DAT≤1+.

169. The method of any of embodiments 151 and 153-166, wherein the subject is selected for administration of the TACI-Fc fusion protein if the subject:
a) has a Pemphigus disease area index (PDAI)≥15;
b) is positive for anti-Dsg1 or anti-Dsg3;
c) is positive for anti-BP180 or anti-BP230; and/or
d) has received stable immunosuppression.

### IX. EXAMPLES

The following examples are included for illustrative purposes only and are not intended to limit the scope of the invention.

### Example 1. Identification of Affinity Modified TACI

This Example describes the generation of mutant DNA constructs of human TACI TNFR domains (TD) for translation and expression on the surface of yeast as yeast display libraries, introduction of DNA libraries into yeast, and selection of yeast cells expressing affinity-modified variants of the extracellular domain (ECD) of TACI containing at least one TD (TACI vTD). The selected TACI vTD were then formatted as Fc fusion proteins.

### A. Generation of Mutant DNA Constructs of TACI TNFR Domains

Libraries containing random substitutions of amino acids were constructed to identify variants of the extracellular domain (ECD) of TAC. Constructs were generated based on a wildtype human TACI sequence containing an ECD portion of TACI that included either (1) both cysteine-rich protein domains (CRD, CRD1/CRD2) as set forth in SEQ ID NO: 1 (corresponding to residues 29-110 as set forth in UniProt Accession No. 014836), or (2) only a single CRD (CRD2) as set forth in SEQ ID NO: 13 (corresponding to residues 68-110 as set forth in UniProt Accession No. 014836).

TACI ECD (29-110) (SEQ ID NO: 1):
VAMRSCPEEQYWDPLLGTCMSCKTICNHQSQRTCAAFCRSLSCRKEQGKF
YDHLLRDCISCASICGQHPKQCAYFCENKLRS
TACI ECD (68-110) (SEQ ID NO: 13):
SLSCRKEQGKFYDHLLRDCISCASICGQHPKQCAYFCENKLRS

DNA encoding the wild-type TACI ECD domain was cloned between the BamHI and KpnI sites of the modified yeast expression vector PBYDS03 (Life Technologies USA) which placed the TACI ECD N-terminal to the yeast surface anchoring domain Sag1 (the C-terminal domain of yeast α-agglutinin) with an in-frame HA fusion tag N-terminal to the TACI ECD sequence and a c-Myc fusion tag C-terminal to the TACI ECD sequence. Expression in this vector is controlled through the inducible GAL1 promoter. After verification of the correct DNA sequence, the wild-type TACI ECD DNA construct was used as template for error-prone PCR to introduce random mutations across the TACI ECD sequence at a frequency of 2-5 mutations per gene copy. The Genemorph II Kit (Agilent, USA) was used in combination with titrating amounts of MnCl2 from 0.0 to 0.6 mM to achieve the desired error rate. After error-prone PCR, the mutagenized DNA was gel purified using the NucleoSpin® Gel and PCR Clean-up kit (Macherey-Nagel, Germany). This isolated DNA fragment was then PCR amplified with OneTaq 2×PCR master mix (New England Biolabs, USA) using primers containing 48 bp overlap regions homologous to pBYDS03 for preparation for large scale yeast electroporation. The TACI ECD DNA insert was gel-purified and resuspended in sterile, deionized water at a nominal concentration of 500 ng/μL.

To prepare the vector for transformation, pBYDS03 was digested with BamHI-HF and KpnI-HF restriction enzymes (New England Biolabs, USA) and the large vector fragment (expected size: 7671 bp) was gel-purified and dissolved in sterile, deionized water at a nominal concentration of 500 ng/μL. To prepare for yeast transformation, 12 μg of library DNA insert was mixed with 4 μg of linearized vector for each electroporation.

To introduce random DNA libraries into yeast, the Saccharomyces cerevisiae strain BJ5464 (ATCC.org; ATCC number 208288) was prepared immediately prior to electroporation as detailed in Benatuil, L. et.al., Protein Eng Des Sel. 2010 April; 23(4):155-159. Briefly, an overnight stationary-phase culture of BJ5464 was passaged to OD₆₀₀ 0.3 in 100 mL YPD medium (10 g/L yeast nitrogen base, 20 g/L Peptone and 20 g/L D-(+)-Glucose) and placed in a platform shaker at 30° C. and 300 rpm until the inoculated cultures reached OD₆₀₀ 1.6. After ~5 hours, cells were harvested by centrifugation and kept on ice for the remainder of the protocol unless otherwise stated. After harvesting, cells were washed twice with 50 mL ice-cold water and once with electroporation buffer (1 M Sorbitol, 1 mM CaCl2)). Collected cells were conditioned by re-suspending in 20 mL 0.1 M LiAc/10 mM DTT and shaking at 225 rpm in a culture flask for 30 minutes at 30° C. Conditioned cells were immediately centrifuged, washed twice with electroporation buffer, and resuspended with ~100-200 μl of electroporation buffer to bring the volume to 1 mL. This conditioned cell suspension was sufficient for two electroporation reactions in 400 μl cuvettes.

For each electroporation, 12 μg of library DNA insert and 4 μg of linearized pBYDS03 vector (described above) was mixed with 400 μl of electrocompetent BJ5464 and transferred to a pre-chilled BioRad GenePulser cuvette with 2 mm electrode gap. The mixtures were kept on ice for 5 minutes, prior to electroporation using a BTX ECM399 exponential decay wave electroporation system at 2500V. Immediately following electroporation, cells were added to 8 mL of 1:1 mixture of 1 M Sorbitol:1×YPD, and left at room temperature without shaking for 10 min, then placed on platform shaker for 1 hr at 225 rpm and 30° C. Cells were collected by centrifugation and resuspended in 250 mL SCD-Leu medium to accommodate the LEU2 selective marker carried by modified plasmid pBYDS03. One liter of SCD-Leu media was generated with 14.7 gm sodium citrate, 4.29 gm citric acid monohydrate, 20 gm dextrose, 6.7 gm yeast nitrogen base, and 1.6 gm yeast synthetic drop-out media supplement without leucine. The medium was filter sterilized before use using a 0.22 m vacuum filter device. Library size was estimated by spotting serial dilutions of freshly recovered cells on an SCD-Leu agar plate in the dilution range of 10⁻⁵ to 10⁻¹⁰ and extrapolating by counting colonies after three days. The remainder of the electroporated culture was grown to saturation and cells from this culture were subcultured 1/100 into the same medium once more and grown to saturation to minimize the fraction of untransformed cells and to allow for segregation of plasmid from cells that may contain two or more library variants. To maintain library diversity, this subculturing step was carried out using an inoculum that contained at least 10× more cells than the calculated library size. Cells from the second saturated culture were resuspended in fresh medium containing sterile 25% (weight/volume) glycerol to a density of 1×10¹⁰/mL and frozen and stored at −80° C. (frozen library stock).

A number of cells equal to at least 10 times the estimated library size were thawed from individual library stocks, suspended to 0.5×10⁷ cells/mL in non-inducing SCD-Leu medium, and grown overnight. The next day, a number of cells equal to 10 times the library size were centrifuged at 2000 RPM for two minutes and resuspended to 0.5×10⁷ cells/mL in inducing SCDG-Leu media. One liter of SCDG-Leu induction media was generated with 5.4 gm Na₂HPO₄, 8.56 gm NaH₂PO₄·H₂0, 20 gm galactose, 2.0 gm dextrose, 6.7 gm yeast nitrogen base, and 1.6 gm yeast synthetic drop out media supplement without leucine dissolved in water and sterilized through a 0.22 m membrane filter device. The culture was grown in induction medium overnight at 30° C. to induce expression of library proteins on the yeast cell surface.

Following overnight induction of the TACI ECD libraries, a number of cells equivalent to 10 times the estimated library diversity were sorted by magnetic separation using Dynabeads™ His-Tag magnetic beads preloaded with BAFF-9×His to enrich for TACI ECD variants with the ability to bind their exogenous recombinant counter-structure proteins. The outputs from the magnetic separation were used in a subsequent FACS selection scheme involving four rounds of positive selections alternating between BAFF-9×His and APRIL-FLAG, with simultaneous 10-fold reduction in counter structure concentration each round (e.g., FACS1: 50 nM APRIL-FLAG; FACS4: 0.05 nM BAFF-9×His). The incubation volume was adjusted to maintain at least a 10-fold stoichiometric excess of counter structure over the total number of yeast-displayed TACI ECD variant molecules (assuming 100,000 copies of protein per cell) to avoid ligand depletion artifacts which can reduce library discrimination. Binding of BAFF-9×His and APRIL-FLAG to TACI ECD variants was detected with PE conjugated anti-6×His tag antibody (BioLegend, USA) and PE conjugated anti-FLAG-tag antibody, respectively. Variants from FACS3 and FACS4 outputs were isolated for DNA sequencing and subsequent cloning for recombinant Fc fusion expression.

A second cycle of random mutagenesis was carried out on yeast cell outputs from the FACS4 BAFF-9×His selections described above. The positive selection protocol with alternating counter structures per sort was the same as the first cycle except that the order of counter structures was switched (e.g., FACS1: 50 nM BAFF-9×His; FACS4: 0.05 nM APRIL-FLAG). Additional variants were chosen from FACS3 and FACS4 yeast cell outputs.

In some embodiments, the FLAG comprises the amino acid sequence set forth in SEQ ID NO:72. In some embodiments, the FLAG is the amino acid sequence set forth in SEQ ID NO:72.

### B. Reformatting Selection Outputs as Fc-Fusions

TACI ECD variant inserts from FACS3 and FACS4 outputs from both cycle 1 and cycle 2 selections, as described above, were subcloned into an Fc fusion vector for sequence analysis of individual clones To generate recombinant immunomodulatory proteins as Fc fusion proteins containing an ECD of TACI with at least one affinity-modified domain (e.g., variant TACI ECD-Fc), the encoding DNA was generated to encode a protein as follows: variant TACI domain followed by a linker of 7 amino acids (GSGGGGS; SEQ ID NO: 74) followed by a human IgG1 effectorless Fc sequence containing the mutations L234A, L235E and G237A, by the Eu Index numbering system for immunoglobulin proteins. Since the construct does not include any antibody light chains that can form a covalent bond with a cysteine, the human IgG1 Fc also contained replacement of the cysteine residues to a serine residue at position 220 (C220S) by Eu Index numbering system for immunoglobulin proteins (corresponding to position 5 (C5S) with reference to the wild-type or unmodified Fc set forth in SEQ ID NO: 71). The Fc region also lacked the C-terminal lysine at position 447 (designated K447del) normally encoded in the wild type human IgG1 constant region gene (corresponding to position 232 of the wild-type or unmodified Fc set forth in SEQ ID NO: 71). The effectorless (inert) IgG1 Fc in the fusion constructs is set forth in SEQ ID NO:73.

Output cell pools from selected TACI ECD FACS sorts were grown to terminal density in SCD-Leu selection medium and plasmid DNA was isolated using a yeast plasmid DNA isolation kit (Zymoresearch, USA). For generation of Fc fusions, the affinity matured TACI ECD variants were PCR amplified with primers containing 40 bp homologous regions on either end with an AfeI and BamHI digested Fc fusion vector encoding and in-frame with the Fc region to carry out in vitro recombination using Gibson Assembly Master Mix (New England Biolabs). The Gibson Assembly reaction was added to the E. coli strain NEB5alpha (New England Biolabs, USA) for heat shock transformation following the manufacturer's instructions.

Dilutions of transformation reactions were plated onto LB-agar containing 100 g/mL carbenicillin (Teknova, USA) to isolate single colonies for selection. Generally, up to 96 colonies from each transformation were then grown in 96 well plates to saturation overnight at 37° C. in LB-broth containing 100 μg/mL carbenicillin (Teknova cat #L8112) and a small aliquot from each well was submitted for DNA sequencing to identify mutation(s) in all clones.

After sequence analysis and identification of clones of interest, plasmid DNA was prepared using the MidiPlus kit (Qiagen).

Recombinant variant Fc fusion proteins were produced from suspension-adapted human embryonic kidney (HEK) 293 cells using the Expi293 expression system (Invitrogen, USA). Supernatant was harvested and the Fc protein was captured on Mab SelectSure (GE Healthcare cat. no. 17543801). Protein was eluted from the column using 50 mM Acetate pH3.6. The MabSelect Sure eluate was pooled and the pH was adjusted to above pH5.0. This material was then polished on a Preparative SEC column, to generate highly purified monomeric material. This material was buffer exchanged into 10 mM Acetate, 9% Sucrose pH 5.0. The protein purity was assessed by analytic SEC. Material was vialed and stored at −80.

Amino acid substitutions in selected TACI vTDs that were identified and generated by the selection are set forth in Table 1. Selected vTDs, formatted as Fc fusion proteins, were tested for binding and functional activity as described in Example 2.

### Example 2. Assessment of Activity of Fc Fusion Proteins

This Example describes characterization of the activity of TACI domain-containing molecules, such as soluble wild-type (WT) or variant TACI vTDs formatted as Fc fusions, using a cell line-based in vitro bioassay.

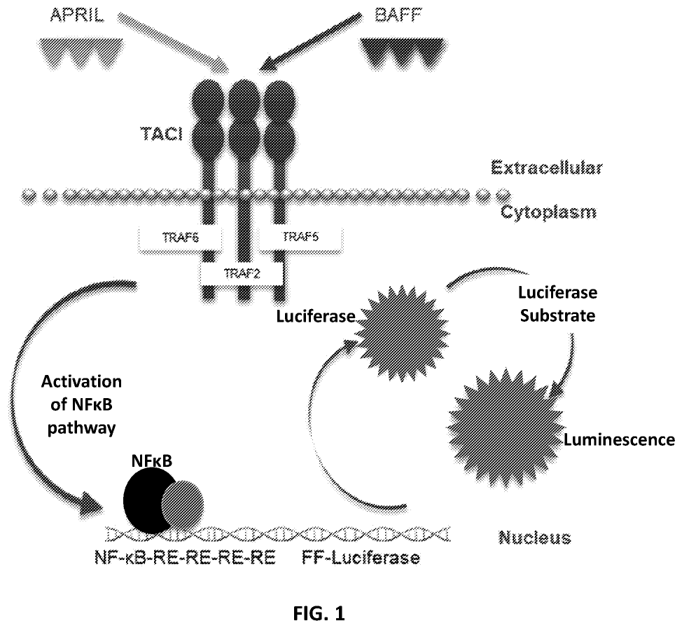

Jurkat cells with a nuclear factor kappa-light-chain-enhancer of activated B cells (NF-κB) luciferase-based reporter were purchased (BPS Bioscience). Jurkat/NK-κB cells were transduced with lentivirus to yield stable, cell surface expression of mouse TACI (Jurkat/NF-κB/TACI). Cells expressing mouse TACI respond to both human and mouse APRIL or BAFF. Following binding of recombinant human or mouse APRIL or BAFF to TACI, endogenous NK-κB transcription factors in the Jurkat cells bind to the DNA response elements controlling transcription of a firefly luciferase gene. Luciferase production was quantitated through the addition of a luciferin-containing substrate which, when oxidized, generates light that can be measured using a microplate reader. A schematic of the Jurkat/NF-κB/TACI assay is shown in FIG. 1.

Recombinant human and mouse APRIL and BAFF ligands were purchased: human APRIL (Tonbo Biosciences); human BAFF (BioLegend); mouse APRIL (ProSci Incorporated); and mouse BAFF (R & D Systems).

To determine bioactivity of TACI WT or vTD domain-containing molecules, recombinant human or mouse APRIL or BAFF at varying concentrations (ranging 1-10 nM) in 30 μL were incubated with fixed or titrated (ranging 40 nM-66 pM) TACI domain-containing molecules in 30 μL. Ligands and soluble receptors were incubated for 20 minutes with shaking at room temperature (RT). Fifty μL was transferred to a 96-well, white flat-bottomed plated containing 1.5×10⁵ Jurkat/NF-κB/TACI cells/well in 50 μL media (RPMI1640+5% fetal bovine serum [FBS]). Wells were mixed and plates incubated for 5 hours at 37° Celsius (C) in a humidified 5% CO₂ incubation chamber. Plates were removed from the incubator and 100 μL of cell lysis and luciferase substrate solution (Bio-Glo™ Luciferase Assay System, Promega) was added to each well and the plates were incubated on an orbital shaker for 10 minutes. Relative luminescence values (RLU) were determined for each test sample by measuring luminescence with a 1 second per well integration time using a Cytation 3 (BioTek Instruments) imaging reader. Decreased RLU in the presence of TACI WT or vTDs relative to control proteins represent blockade and inhibition of ligand signaling via the transduced TACI receptor in the Jurkat/NF-κB/TACI cells.

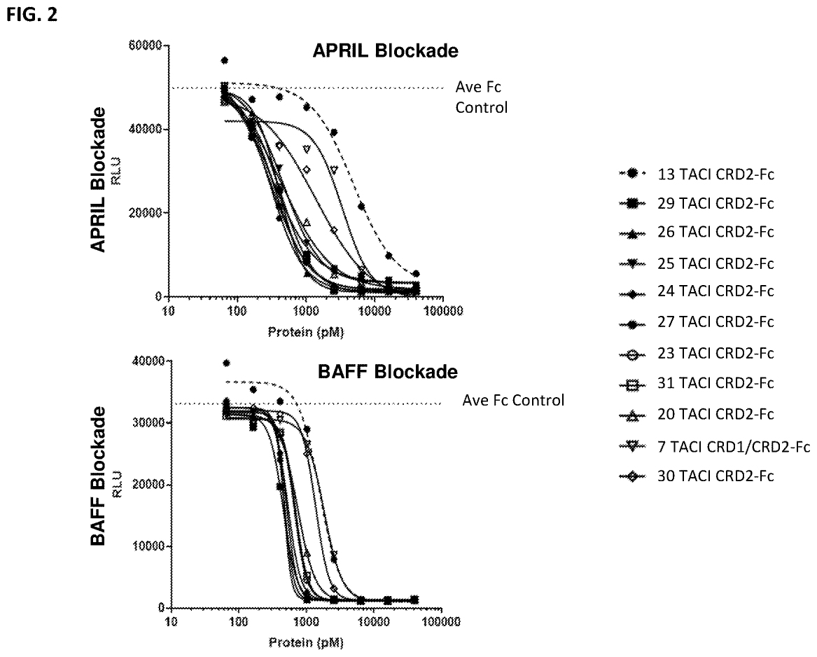

As shown in FIG. 2, exemplary TACI-Fc vTDs, respectively, inhibit ligand signaling at levels equal to or greater than Fc fusion proteins containing WT TACI domains.

Similar experiments also were conducted to additionally assess functional blockade of cynomolgus monkey and rat APRIL- or BAFF-mediated signaling by the exemplary TACI-Fc fusion containing a vTD set forth in SEQ ID NO:26 (26 TACI CRD2-Fc) using a Jurkat/NF-κB luciferase reporter cells transduced with TACI, substantially as described above. As shown in Table E1.A, the TACI-Fc fusion demonstrated blockade of APRIL- and BAFF-mediated signaling for all species tested.

TABLE E1.A
Cross-Species Inhibition of 26 TACI CRD2-Fc

| | Signal Inhibition in Jurkat/NF-κB/TACI cells | |
|---|---|---|
| Species of BAFF/APRIL | APRIL IC₅₀ (nM) | BAFF IC₅₀ (nM) |
| Human | 3.4 | 2.8 |
| Cynomolgus monkey | 3.3 | 1.9 |
| Rat | 22.7 | 3.8 |
| Mouse | 24.5 | 15.2 |

Notes:
APRIL = A proliferation inducing ligand; BAFF = B cell activating factor; IC₅₀ = Half Maximal inhibitory concentration; NF-κB = Nuclear factor kappa-light-chain-enhancer of activated B cells; TACI = Transmembrane activator and calcium modulating ligand interactor.

### Example 3. Binding and Bioactivity Assessment of TACI Blockade of TACI-Mediated Stimulation by TACI-Containing Molecules

Binding and bioactivity assays were used to further assess the TACI-Fc fusion proteins and other comparators.

### A. Bioactivity Assessment

The cell-line based bioassay described in Example 2 was used to assess the functional characterization of TACI-containing WT or vTD proteins for blockade of APRIL or BAFF-mediated ligand signaling via the TACI receptor in the Jurkat/NF-κB/TACI cells. APRIL or BAFF-mediated ligand signaling was quantitated by monitoring luciferase production in the cells. Binding of a TACI-Fc fusion containing a vTD set forth in SEQ ID NO:26 was assessed (26 TACI CRD2-Fc). For comparison, WT TACI-Fc containing only the CRD2 domain of TACI (13 TACI CRD2-Fc) also was assessed.

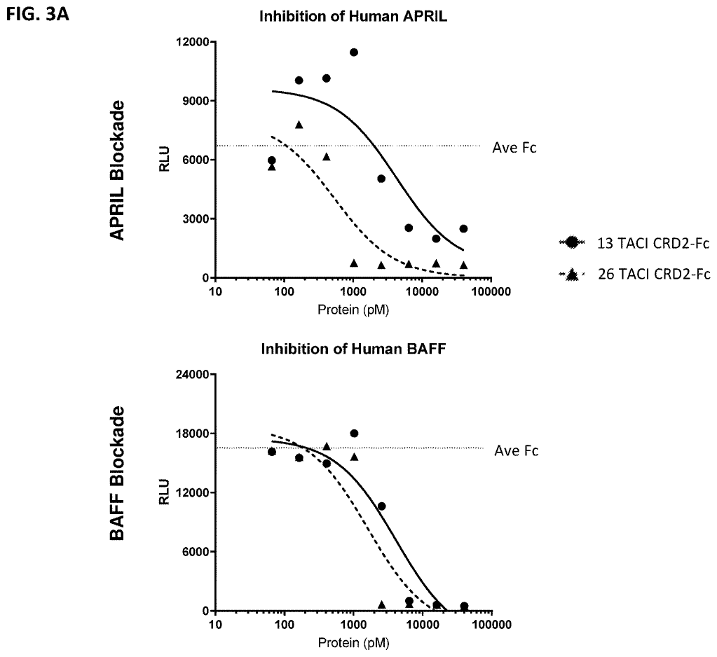

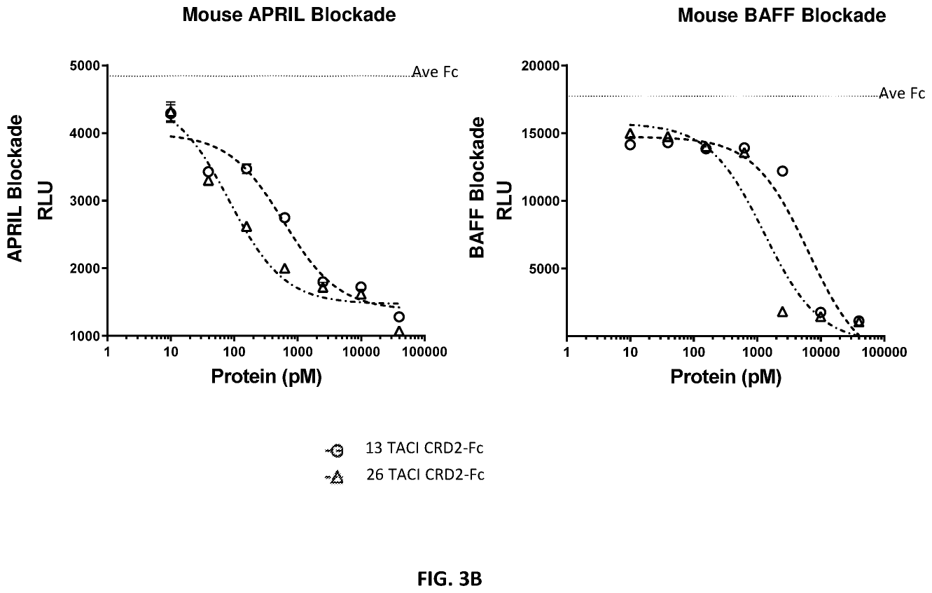

As shown in FIG. 3A, an exemplary TACI vTD demonstrates increased inhibition of both human APRIL and BAFF. As shown in FIG. 3B, exemplary TACI vTD-Fc molecules inhibit mouse APRIL and BAFF ligand signaling. Together, the results show the ability of TACI vTD molecules to block APRIL and BAFF TACI-mediated ligand signaling.

In another similar study, exemplary generated molecules as described in Example 1 were assessed for their ability to block APRIL or BAFF-mediated ligand signaling in a reporter assay using Jurkat/NF-κB/TACI cells. For comparison, control molecules were generated containing wild-type TACI ECD fused the Fc sequence set forth in SEQ ID NO: 73. In one control, the fusion protein contained WT TACI (TACI 30-110, SEQ ID NO:130; corresponding to the TACI ECD portion in atacicept, SEQ ID NO:132). In another control, the fusion protein contained WT TACI (TACI 13-118, SEQ ID NO:131), corresponding to the TACI ECD portion in telitacicept). The tested molecules also included 13 TACI-CRD2-Fc containing only the CRD2 domain, which was compared to WT TACI 30-110-Fc (e.g. atacicept) and WT TACI 13-118-Fc (e.g. telitacicept), each of which contain both CRD1 and CRD2. Activity was compared to the control molecules. Activity also was compared to the anti-BAFF monoclonal antibody belimumab.

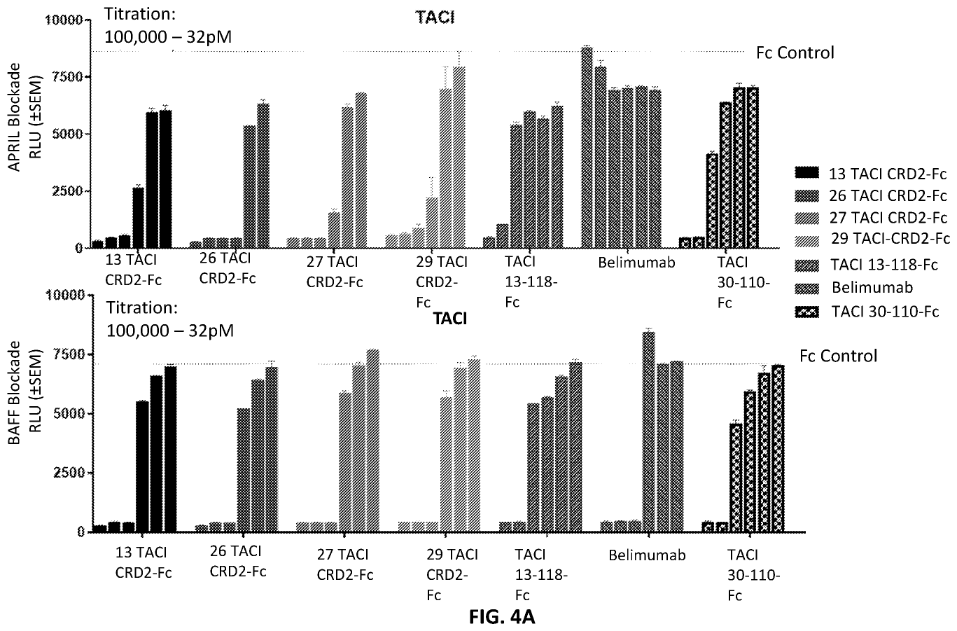

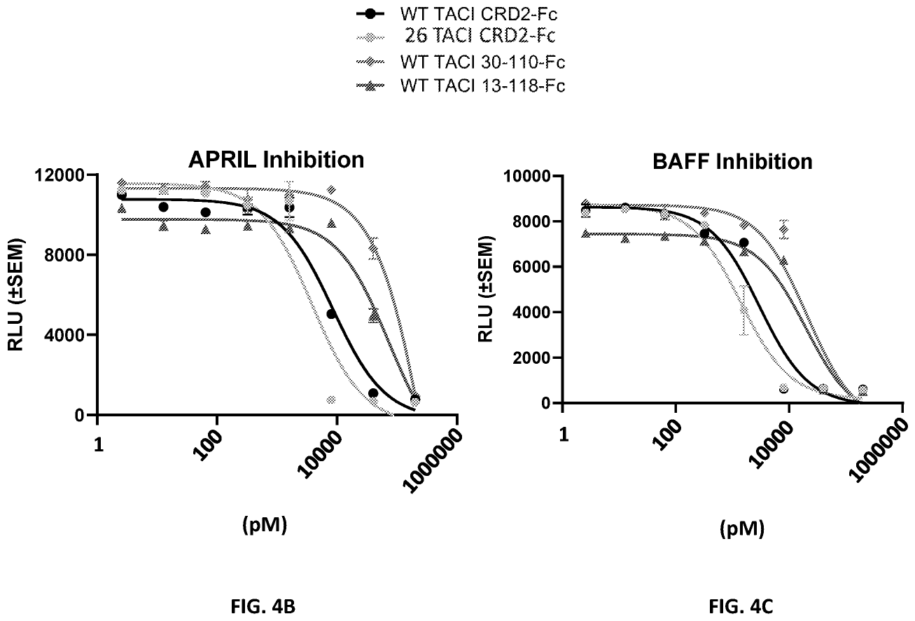

Exemplary TACI molecules, either WT or variant TACI vTDs, were titrated (between 100,000 pM-32 pM), added to 2 nM recombinant human APRIL or BAFF and assayed as described above for the Jurkat/NF-κB assay. As shown in FIG. 4A, the exemplary molecules containing TACI vTDs exhibited enhanced APRIL and BAFF blockade greater than TACI 30-100-Fc, TACI 13-118-Fc and belimumab. WT TACI-Fc containing only the CRD2 domain of TACI (13 TACI CRD2-Fc) also exhibited enhanced APRIL blockade greater than TACI 30-100-Fc and TACI 13-118-Fc. Binding curves are shown in FIGS. 4B and 4C, in which increased inhibitory activity is indicated by reduced luciferase binding.

These results are consistent with a finding that the minimal CRD2 domain (containing amino acids residues 68-110) exhibits improved blockade of APRIL compared to TACI ECD molecules also containing portions of the CRD1 domain as present in atacicept and telitacicept. Table E1.B provides the values for half maximal inhibitory concentration (IC50) for inhibition of APRIL- and BAFF-mediated TACI signaling for the exemplary molecules described in FIG. 4A. Also shown in parentheses is the relative blockage compared to atacicept (A atacicept) for each tested molecule.

TABLE E1.B
Bioactivity of TACI vTDs vs atacicept

| | | | IC50 (nM) APRIL | IC50 (nM) BAFF |
|---|---|---|---|---|
| | SEQ | IC50 (nM) | (Δ TACI | (Δ TACI |
| Description | ID NO | APRIL | 30-110-Fc) | 30-110-Fc) |
| 26 TACI CRD2-Fc | 26 | 179 | 179(0.05) | 1216(0.21) |
| 27 TACI CRD2-Fc | 27 | 262 | 262(0.07) | 1387(0.24) |
| 29 TACI CRD2-Fc | 29 | 339 | 339(0.09) | 1336(0.23) |
| 13 TACI CRD2-Fc | 13 | 369 | 369(0.10) | 1328(0.23) |
| TACI 13-118-Fc | | 9103 | 9103(2.37) | 7699(1.33) |
| Belimumab | | 214911 | 214911(55.84) | 2496(0.43) |
| TACI 30-110-Fc | | 3849 | 3849(1.00) | 5771(1.00) |

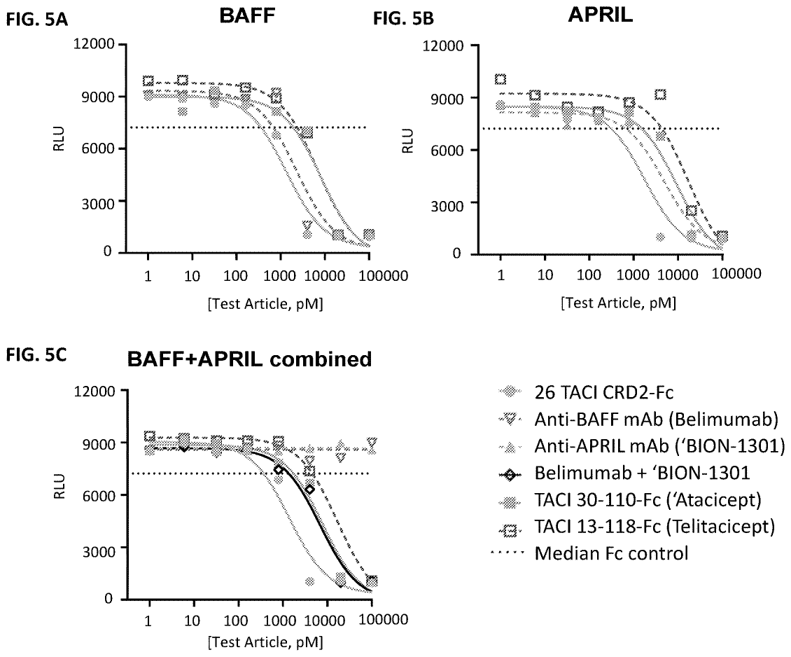

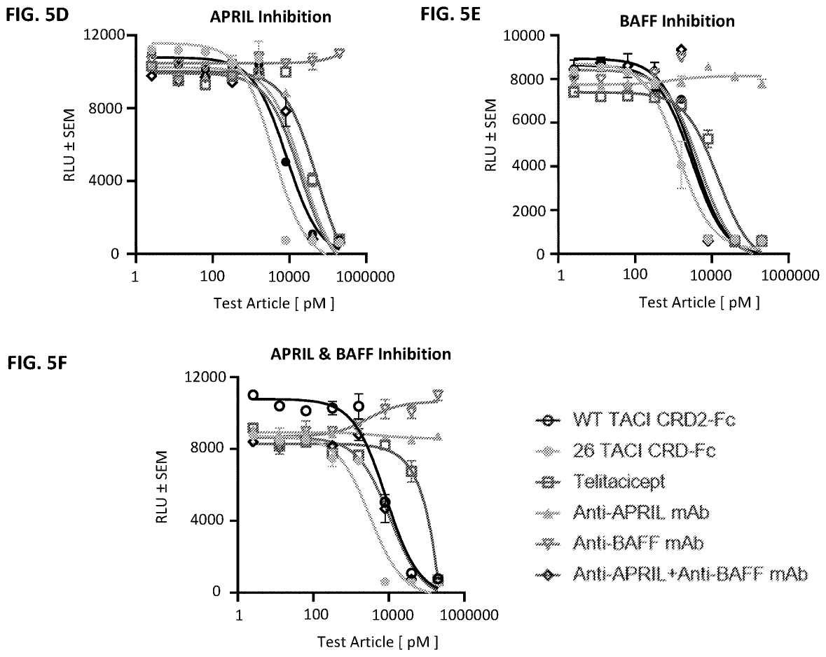

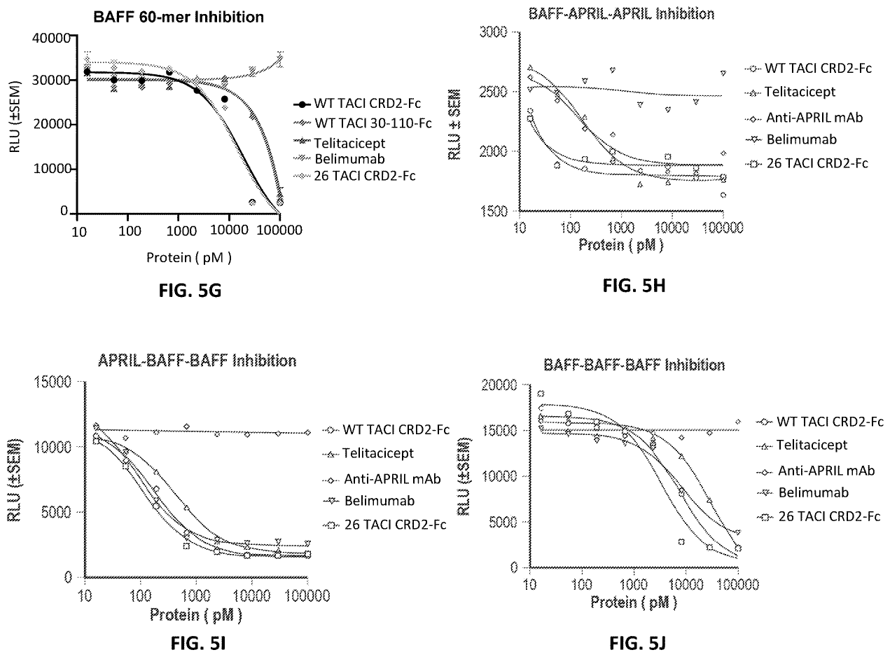

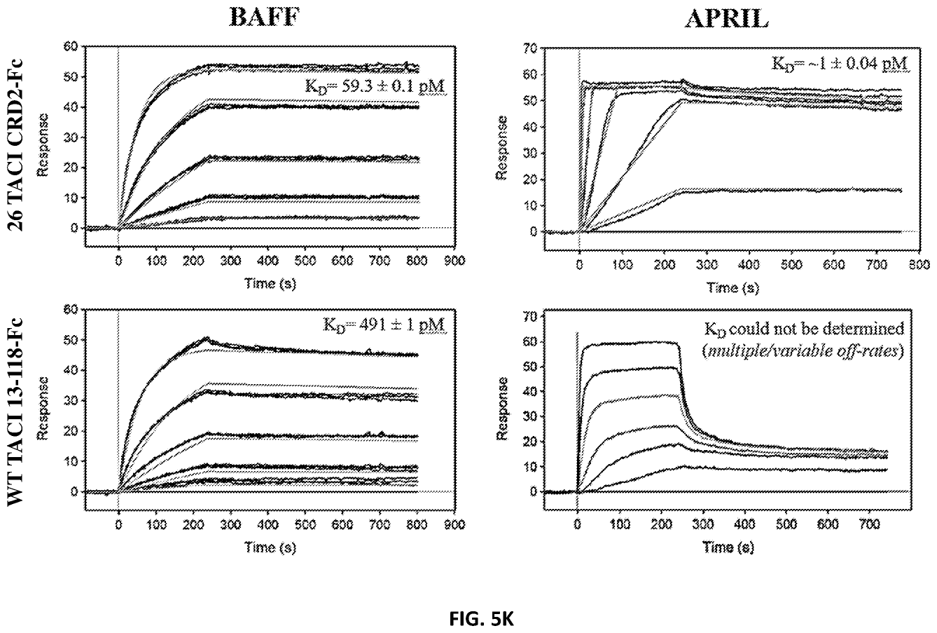

In another study, the bioactivity of vTD domain-containing molecule 26 TACI CRD2-Fc (containing a vTD TACI domain set forth in SEQ ID NO:26; Fc fusion SEQ ID NO: 167) were assessed in the cell line-based in vitro bioassay described in Example 2 in the presence of recombinant human or mouse APRIL and/or BAFF, either independently or in combination. Varying concentrations of TACI domain-containing molecules were incubated with 15 nM APRIL, 10 nM BAFF or APRIL+BAFF (15 nM APRIL+10 nM BAFF) in combination. Activity was compared to the control molecules. For comparison, WT TACI-Fc sequences corresponding to atacicept (containing a WT TACI 30-110 SEQ ID NO:132; SEQ ID NO:130) or Telitacicept/Tai'ai (RemeGen) were tested. As further controls, varying concentrations of anti-BAFF monoclonal antibody (mAb) containing sequences from belimumab (Benlysta) or anti-APRIL mAb BION-1301 (e.g. SEQ ID NO: 50 and 52 from U.S. Pat. No. 10,377,830), each singly or together, also were incubated with APRIL, BAFF or BAFF+APRIL in combination. As shown in FIGS. 5A-5C and Table E1.C, exemplary 26 TACI CRD2-Fc inhibited ligand signaling at levels equal to or greater than the Fc fusion proteins atacicept, telitacicept, as well as belimumab or BION-1301 individually or combined. The fusion protein containing 26 TACI CRD2-Fc neutralized the combined activity of BAFF and APRIL.

In a similar experiment, recombinant human APRIL or BAFF (1-10 nM) were incubated with fixed or titrated (200 nM-2.5 pM) TACI domain-containing molecules or comparators. Ligands and inhibitors or Fc controls were incubated for 20 min shaking (150 rpm) at room temperature (RT) prior to the addition of TACI/Jurkat/NF-κB reporter cells described in Example 2 (1.5×10⁵cells/well) within a white 96-well flat bottom plate. Plates were incubated 5 hours at 37° C., after which luciferase substrate solution (Bio-Glo™ Luciferase Assay System, Promega) was added while shaking (150 rpm) for 10 minutes. Relative luminescence units (RLU) were determined using a Cytation 3 (BioTek Instruments) or SpectraMax iD3 (Molecular Devices) imaging reader. Results are shown in FIG. 5D (APRIL inhibition), FIG. 5E (BAFF inhibition) and FIG. 5F (APRIL plus BAFF inhibition). IC50 values for inhibition are shown in Table E1.D. 26 TACI CRD2-Fc effectively neutralized APRIL 5.7-fold better than the anti-APRIL mAb, and neutralized BAFF 3.4-fold better than belimumab. 26 TACI CRD2-Fc was also the most potent inhibitor of the combination of APRIL and BAFF, with a >50-fold lower IC50 value relative to telitacicept, and a 3.5-fold better IC50 value relative to the combination of anti-APRIL mAb plus belimumab.

These results support that the variant TACI-Fc, 26 TACI CRD2-Fc, neutralizes APRIL and BAFF activity more potently than WT TACI-Fc or combined anti-BAFF+APRIL mAbs in a cell-based reporter assay.

TABLE E1.C
IC50 values

| | IC50 (pM) | | |
|---|---|---|---|
| Test Articles | BAFF | APRIL | BAFF + APRIL |
| 26 TACI CRD2-Fc | 1337 | 1758 | 1390 |
| Belimumab | 2263 | — | >100,000 |
| BION-1310 | — | 4901 | >100,000 |
| Belimumab + BION-1310 | 6848 | 2100 | 6805 |
| Atacicept | 8285 | 9258 | 7718 |
| Telitacicept | 6852 | 17244 | 16751 |

TABLE E1.D
TACI/Jurkat/NF-κB BAFF and APRIL inhibitory assay IC₅₀ values

| | Human IC₅₀ (nM) | | | Mouse IC₅₀ (nM) | |
|---|---|---|---|---|---|
| Test Article Protein | APRIL | BAFF | APRIL + BAFF | APRIL | BAFF |
| 13 TACI CRD2-Fc | 8.5 | 3.0 | 12.9 | 2.9 | 2.2 |
| 26 TACI CRD2-Fc | 3.8 | 1.4 | 3.1 | 0.9 | 1.0 |
| WT TACI | >200 | 20.8 | >200 | 18.0 | 9.0 |
| 30-110-Fc¹ | | | | | |
| WT TACI | 73.1 | 19.6 | >200 | 14.3 | 9.4 |
| 13-118-Fc² | | | | | |
| Telitacicept³ | 53.9 | 14.5 | >200 | 15.5 | 7.9 |
| anti-APRIL mAb⁴ | 21.9 | CNBD | >200 | — | — |
| Anti-APRIL mAb⁶ | 5.1 | CNBD | CNBD | — | — |
| belimumab⁵ | CNBD | 4.8 | CNBD | — | — |
| anti-APRIL mAb + | 18.9 | 4.8 | 10.8 | — | — |
| belimumab | | | | | |

¹WT TACI-Fc based on the published atacicept sequence;
²WT TACI-Fc based on the published telitacicept sequence;
³Telitacicept sourced through Clinigen;
⁴Anti-APRIL mAb based on the published BION-1301 sequence. Does not bind mouse APRIL.
⁵Belimumab sourced through Myonex. Does not bind mouse BAFF. CNBD, could not be determined.
⁶Anti-APRIL mAb based on published sibeprenlimab sequence.

26 TACI CRD2-Fc was tested for its ability to inhibit BAFF 60-mer- and APRIL/BAFF heterotrimer-induced signaling in the TACI/Jurkat/NF-κB assay described in Example 2. APRIL and BAFF can form heterotrimeric proteins at 2:1 ratios (Roschke, Sosnovtseva et al. 2002, Dillon, Harder et al. 2010). Additionally, soluble BAFF exists in a high molecular weight complex designated as BAFF 60-mer (Nicoletti, Kenny et al. 2016, Eslami, Meinl et al. 2020). Inhibition of APRIL and BAFF trimers/heterotrimers as well as BAFF 60-mer is likely required for full inhibition of these pathways in autoimmune settings where multiple forms of these ligands can be present and/or elevated (Eslami and Schneider 2021).

26 TACI CRD2-Fc inhibited BAFF 60-mer comparably to WT TACI CRD2-Fc and significantly better (p<0.0001) than the full-length WT TACI-Fc proteins tested (FIG. 5G, Table E1.E). In addition, single chain heterotrimers of APRIL and BAFF were generated, consisting of BAFF APRIL APRIL (BAA) and ABB formats to represent both possible forms (Schuepbach-Mallepell, Das et al. 2015). A single chain BAFF homotrimer was included as a control. 26 TACI CRD2-Fc demonstrated greater inhibition against the BAFF homotrimer and BAFF/APRIL heterotrimers as compared to telitacicept (FIGS. 5H-5J, Table E1.E). An anti-APRIL antibody produced from the published sequence for VIS649 (sibeprenlimab) only inhibited the BAA heterotrimer, while belimumab inhibited the ABB heterotrimer (in contrast to previously published results (Schuepbach-Mallepell, Das et al. 2015) and the BAFF homotrimer. Collectively, these findings indicate that directed engineering of a single TACI CRD2 domain is sufficient to generate significantly enhanced affinity for APRIL and increased BAFF affinity, resulting in inhibitory activity superior to clinically relevant therapeutics, including monoclonal antibodies.

TABLE E1.E
Assessment of Exemplary Monomeric and Tetrameric Immunomodulatory Proteins

| | Human IC₅₀ (pM) | | | |
|---|---|---|---|---|
| | | BAFF-APRIL- | APRIL-BAFF- | BAFF-BAFF- |
| Test Article Protein | BAFF 60-Mer | APRIL | BAFF | BAFF |
| TACI CRD2-Fc | 14669 | <16 | 103 | 3363 |
| WT TACI CRD2-Fc | 18711 | <16 | 179 | 7458 |
| WT TACI 30-110-Fc | >100000 | NT | NT | NT |
| Telitacicept¹ | >100000 | 173 | 408 | 30794 |
| Anti-APRIL mAb² | NT | 144 | CNBD | CNBD |
| Belimumab³ | CNBD | CNBD | 90 | 8524 |

¹Telitacicept sourced through Clinigen;
²Anti-APRIL mAb based on the published VIS649 (sibeprenlimab) sequence.
³Belimumab sourced through Myonex.
Does not bind mouse BAFF;
NT = Not Tested
CNBD = Could Not Be Determined

### B. Binding Affinity

Affinity measurements of 26 TACI CRD2-Fc and WT TACI-Fc binding to recombinant human BAFF and APRIL was determined by surface plasmon resonance (SPR). Exemplary SPR sensorgrams and non-linear least squares regression analysis of the data are shown in FIG. 5K. Results demonstrated that the exemplary 26 TACI CRD2-Fc exhibited significantly enhanced affinity for BAFF and APRIL compared to WT TACI 113-118-Fc as shown by the dissociation constant (K_{D}). 26 TACI CRD2-Fc exhibited K_{D} for BAFF that was 59.3±0.1 pM and the K_{D} for APRIL that was ~1±0.04 pM. The K_{D} of WT TACI 113-118-Fc for BAFF was 491 pM and the K_{D} for binding APRIL could not be determined due to multiple on/off rates, demonstrating WT TACI 113-118-Fc indicated poor retention for APRIL, with rapid and variable K_{off}. The results are summarized in Table E1.F. The surface plasmon resonance results demonstrate that the affinity of 26 TACI CRD2-Fc for BAFF (59.3 pM) was approximately 8-fold better than that of telitacicept (491 pM); its affinity for APRIL was approximately 1 pM.

TABLE E1.F
Affinity measurements of 26 TACI CRD2-Fc and telitacicept binding
to recombinant human APRIL and BAFF as determined by SPR

| Test Article | Sample | k_{a app} 1 × 10⁹ (M⁻¹s⁻¹) | k_{d app} 1 × 10⁻³ (s⁻¹) | Rₘₐₓ (RU) | K_{D app} (pM) |
|---|---|---|---|---|---|
| 26 TACI | APRIL | 7.0 ± 7 | 7.0 ± 6 | 55.5 ± 0.1 | 1.00 ± 0.04 |
| CRD2-Fc | | | | | |
| Telitacicept | APRIL | CNBD | CNBD | CNBD | CNBD |
| Test Article | Sample | kₐ 1 × 10⁵ (M⁻¹s⁻¹) | k_{d} 1 × 10⁻³ (s⁻¹) | Rₘₐₓ (RU) | K_{D} (pM) |
| 26 TACI | BAFF | 6.19 ± 0.01 | 3.67 ± 0.02 | 52.8 ± 0.3 | 59.3 ± 0.1 |
| CRD2-Fc | | | | | |
| Telitacicept | BAFF | 1.75 ± 0.01 | 8.63 ± 0.02 | 47.6 ± 0.4 | 491 ± 1 |

### Example 4. Assessment of the Activity of TACI vTD-Fes in an In Vivo Mouse Lupus Model

This Example describes the assessment of exemplary TACI vTD-Fc molecules, to affect immune responses in an in vivo murine (NZB/NZW)F1 spontaneous lupus model. (NZB×NZW)F1 mice spontaneously develop an autoimmune disease very similar to human SLE and are regarded as one of the best mouse models of this disease. (NZB/NZW)F1 mice have high circulating concentrations of anti-dsDNA antibodies starting around 20 weeks of age, with the first clinical signs of disease detectable around 23 weeks of age. The mice develop hemolytic anemia, proteinuria, and progressive glomerulonephritis mediated by immune complex deposition in the glomerular basement membrane.

(NZB/NZW)F1 mice were dosed twice weekly via intraperitoneal (IP) injection with 14 mg/kg Fc control, or molar-matched amounts of TACI vTD-Fc (26 TACI CRD2-Fc) (17 mg/kg). Treatment started at group assignment (Week 22 of age) and continued through the end of the study. The study ended when mice reached Week 43 of age, though some animals were euthanized earlier in the study when they became moribund.

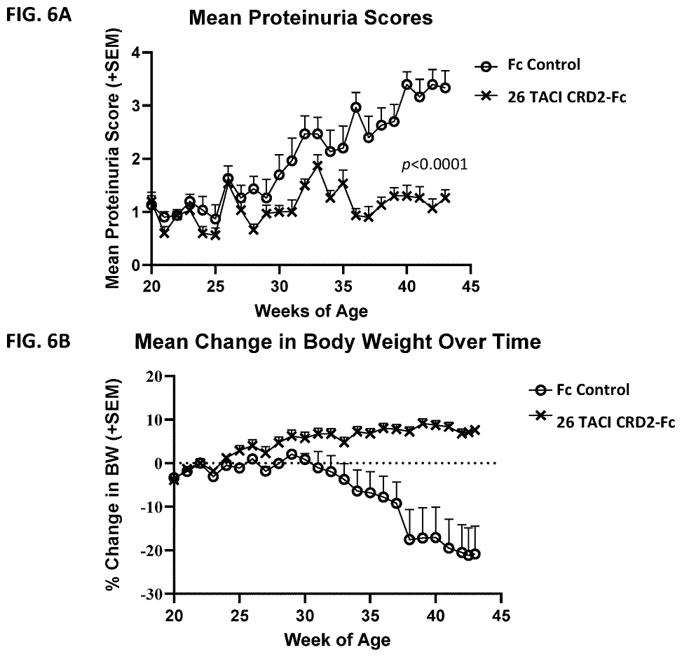

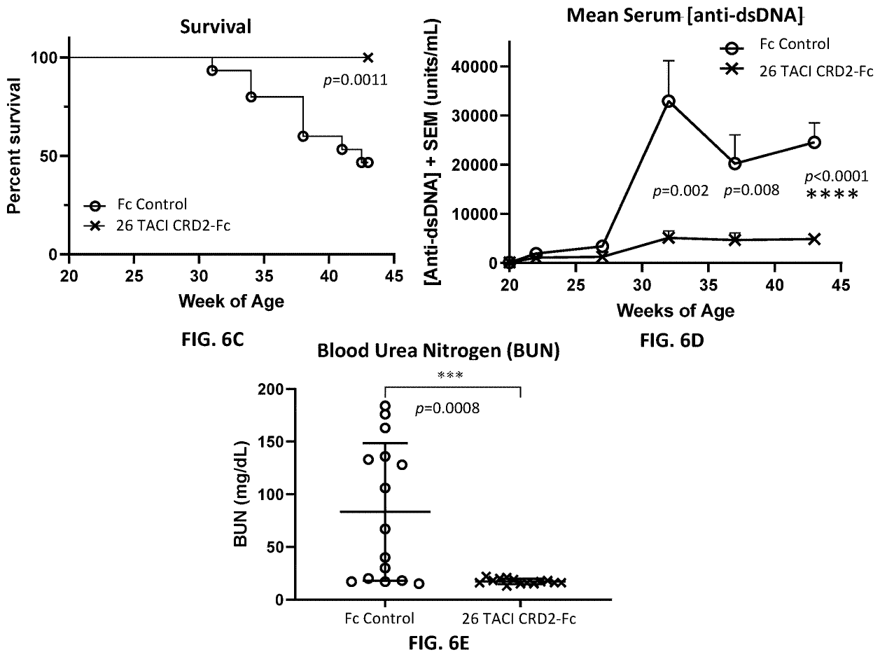

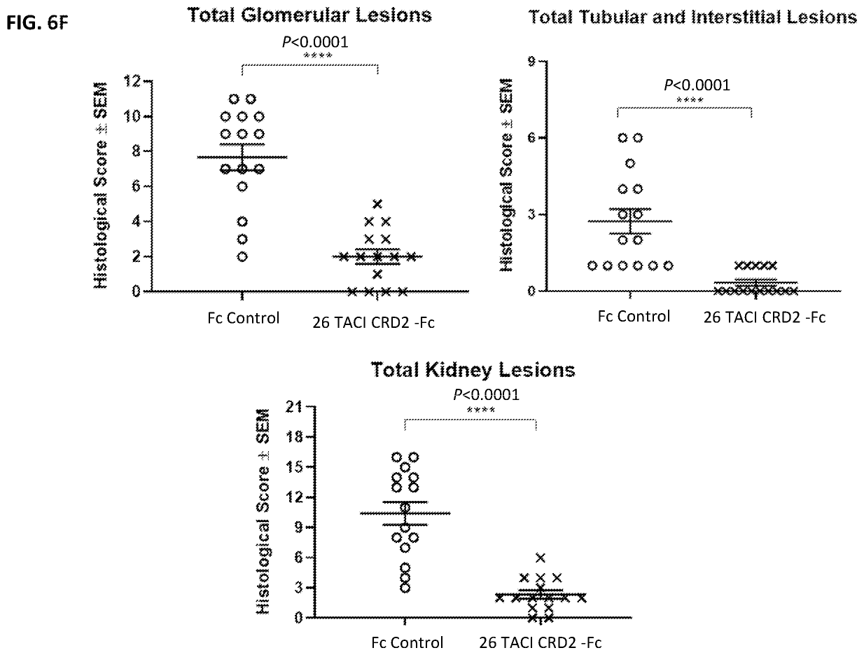

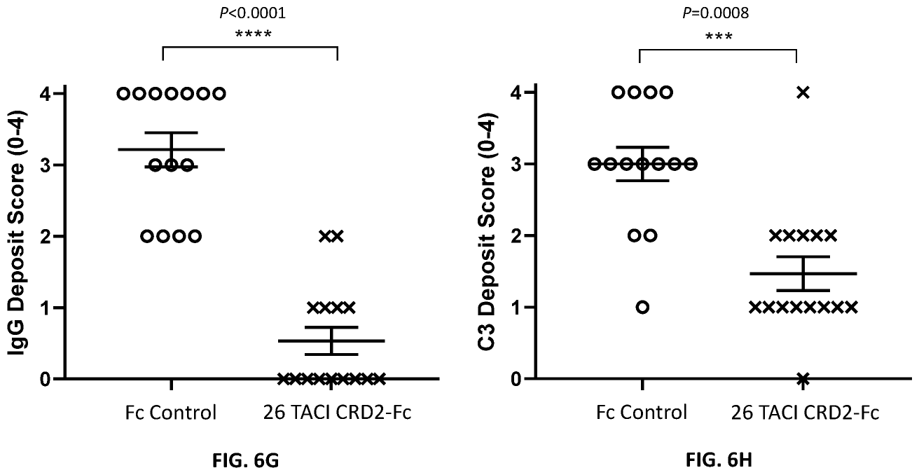

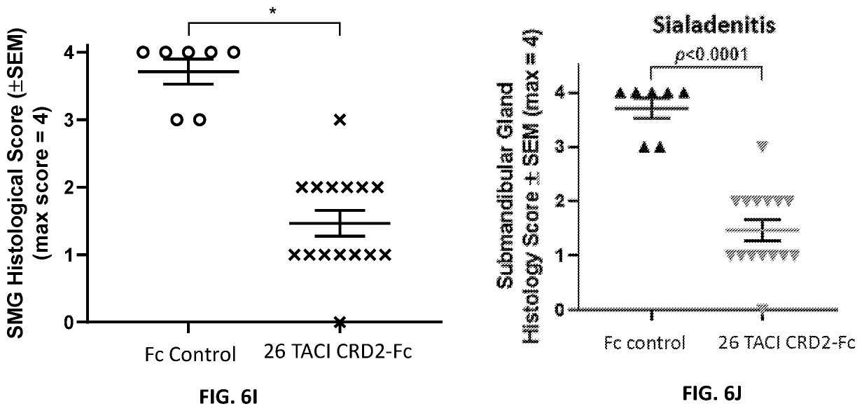

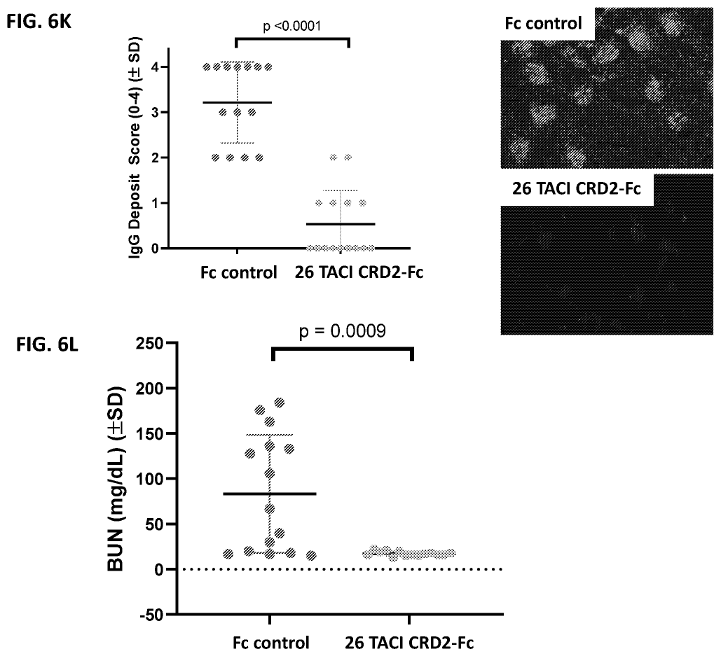

At various time points between 20 and 40 weeks of age, urine and serum samples were collected. Starting when mice were 20 weeks old, the concentration of protein in the urine from all mice on study was determined weekly with urinalysis test strips (Roche Chemstrip 2 GP, cat. 11895397160). Mean proteinuria scores over time in each treatment group are presented in FIG. 6A, and the mean percent change in body weight (weight loss is associated with advancing disease) in each group in plotted in FIG. 6B. The percent survival of mice in each treatment group is plotted in FIG. 6C. Anti-double stranded (ds) DNA IgG serum titers were measured by Hooke Laboratories, Inc. (Lawrence, MA) using their in-house kit, and the results are presented in FIG. 6D. Blood urea nitrogen (BUN) levels increase in these mice with advancing disease. BUN levels at termination of the study (or at sacrifice of mice that succumbed early) for each treatment group are shown in FIG. 6E, see also 6L Statistical analysis was performed using Student's t-test; **** denotes p<0.0001 and *** denotes p=0.0008).

Kidneys were collected at termination from each mouse and analyzed histologically in replicate Periodic acid-Schiff (PAS)-stained sections using the criteria described in Alperovich G et al, 2007. Lupus 16:18-24. All kidney sections were analyzed blind, by a pathologist unaware of the treatments and clinical scores. Glomerular lesions (mesangial expansion, endocapillary proliferation, glomerular deposits, and extracapillary proliferation) and tubular/interstitial lesions (interstitial infiltrates, tubular atrophy, and interstitial fibrosis) were analyzed and graded semi-quantitatively using a scoring system from 0 to 3, with 0=no changes, 1=mild changes, 2=moderate changes, and 3=severe changes. A total histological score for each mouse was calculated as the sum of the individual scores (maximum total score is 21). Kidney scores for total glomerular lesions, total tubular and interstitial lesions, and total kidney lesions are shown in FIG. 6F; as compared to Fc control treated mice, significantly improved renal histopathology was observed in animals treated with TACI vTD-Fc (p<0.0001 vs. Fc group).

For FIG. 6G-6I, the right kidney was collected from each mouse at study termination, weighed, dissected transversally, and frozen in a single optimal cutting temperature compound (OCT) block before sectioning and immunohistochemical (IHC) staining of mouse IgG and mouse complement C3 to assess glomerular IgG and C3 deposition, respectively. The kidney sections were permeabilized with acetone and stained with FITC-conjugated rat monoclonal anti-mouse complement component C3 (Cedarlane) diluted 1:25 in Primary Antibody Diluent (Leica Biosystems), or AF594-conjugated goat anti-mouse IgG (Thermo Fisher Scientific) diluted 1:200 in Primary Antibody Diluent. Glomerular depositions of IgG and C3 were analyzed by a pathologist using a semiquantitative scoring system from 0 to 4, with 0=no deposits, 1=mild mesangial deposition, 2=marked mesangial deposition, 3=mesangial and slight capillary deposition, and 4=intense mesangial and mesangiocapillary deposition, based on the method described in Kelkka et al. (2014) Antioxid Redox Signal. 21:2231-45. As compared to Fc control treated mice, significantly reduced glomerular IgG and C3 were observed in animals treated with 26 TACI CRD2-Fc (p<0.0001 vs. Fc control group for IgG, and p=0.0005 for C3); data were analyzed for statistically significant differences using Student's t-test. As compared to Fc control, 26 TACI CRD2-Fc also reduced sialadenitis (FIG. 6J; (p<0.0001 vs. Fc control group).

Moreover, 26 TACI CRD2-Fc significantly reduced deposition of renal IgG (FIG. 6K).

Thus, together results showed that compared to Fc control, 26 TACI CRD2-Fc reduced anti-double-stranded (ds) DNA autoantibodies, sialadenitis, glomerulonephritis, BUN, proteinuria, and mortality.

Results demonstrate that the TACI vTD-Fc were able to significantly suppress proteinuria, preserve body weight, enhance overall survival, reduce anti-dsDNA autoantibodies and BUN, reduce IgG and C3 renal deposits, and prevent or improve kidney disease in the (NZB/NZW)F1 mouse model of SLE. Exemplary molecules were also capable of potently reducing B and T cell subsets including plasma cells, follicular T helper cells, germinal center cells, and memory T cells in the spleens and lymph nodes of these mice (data not shown).

### Example 5: Assessment of Activity of TACI 13-118-Fc with the Addition of Identified Mutations

The impact of TACI mutations identified in Example 1 (see Table 1) were assessed to determine their ability to modulate the activity of Fc fusion proteins containing a longer TACI ECD sequence (containing both the CRD1 and CRD2 domain). In this example, the exemplary mutations K77E, F78Y and Y102D were introduced into the reference TACI ECD 13-118, which was fused to the exemplary Fc sequence set forth in SEQ ID NO:73. Activity was compared to a TACI vTD-Fc fusion protein containing only the CRD2 domain with the same mutations (set forth in SEQ ID NO:26), or to WT TACI (30-110, SEQ ID NO:130; corresponding to the TACI ECD portion in atacicept, SEQ ID NO:132), each also fused to the Fc sequence set forth in SEQ ID NO:73. The cell line-based bioassay described in Example 2 was used to assess blockade of APRIL or BAFF-mediated ligand signaling via the TACI receptor in the Jurkat/NF-κB/TACI cells. APRIL or BAFF-mediated ligand signaling via the TACI receptor was quantitated by monitoring luciferase production in the cells.

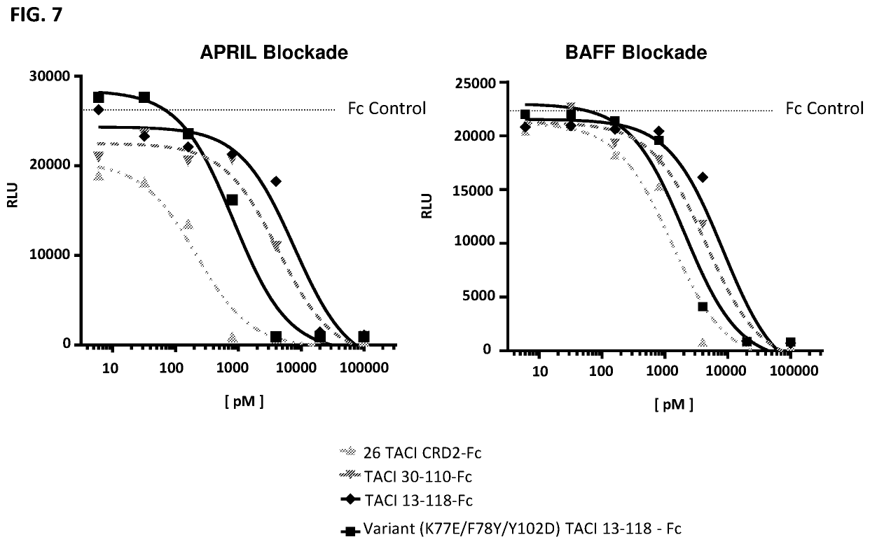

As shown in FIG. 7, introduction of K77E, F78Y and Y102D mutations into TACI 13-118 ECD to generate variant (K77E/F78Y/T102D) TACI 13-118 improved APRIL and BAFF blockade (respectively) relative to the corresponding WT TACI 13-118ECD (diamonds) or the alternative ECD control WT TACI 30-110 (upward triangles). However, even with the incorporation of the mutations into TACI 13-118 ECD, the shorter variant TACI with the same mutations but containing only the CRD2 domain of TACI (vTD set forth in SEQ ID NO:26) exhibited the greatest APRIL and BAFF blockade in this assay (downward triangles). These results confirm that a minimal CRD2-containing domain confers improved activity to block APRIL and BAFF-mediated TACI signaling, however, the mutations K77E/F78Y/Y102D also further enhance APRIL and BAFF blockade by variant TACI ECDs incorporating the mutations.

Table E2 provides the values for half maximal inhibitory concentration (IC50) for inhibition of APRIL- and BAFF-mediated TACI signaling for the exemplary molecules described in FIG. 7. Also shown is a comparison to WT TACI-Fc controls (A atacicept) for each molecule.

TABLE E2
Bioactivity of Multi-Domain Immunomodulatory Proteins vs atacicept

| | | | IC50 (nM) | | IC50 (nM) |
|---|---|---|---|---|---|
| | SEQ ID | IC50 (nM) | APRIL | IC50 (nM) | BAFF |
| Description | NO | APRIL | (Δ TACI 30-110) | BAFF | (Δ TACI 30-110) |
| 26 TACI-Fc | 26 | 214 | 214(0.05) | 1268 | 1268(0.28) |
| TACI 13-118 | 131 | 7811 | 7811(1.81) | 8452 | 8452(1.88) |
| TACI 13-118, with | | 848 | 848(0.20) | 2048 | 2048(0.46) |
| K77E/F78Y/Y102D | | | | | |
| TACI 30-110 | | 4317 | 4317(1.00) | 4490 | 4490(1.00) |

### Example 6: Comparative Evaluation of TACI vTD-Fes in an In Vivo KLH Immunization Model

This Example describes the assessment of exemplary tested single domain Fc fusion proteins (described in Example 1) to affect immune responses to keyhole limpet hemocyanin (KLH) in vivo in mice. The mouse KLH immunization model can be used to evaluate the effects of the immunomodulatory molecules on antigen-specific responses to the T cell-dependent antigen KLH, following either one or two injections of KLH. Two injections of KLH, each separated by at least 7 days, provides a model that can evaluate both a primary immune response following the 1^{st} KLH injection, and a secondary immune response in the period following the 2^{nd} injection. This Example describes a study that evaluated the activity of multiple TACI single domain-containing molecules, such as soluble wild-type (WT) or variant TACI vTDs formatted as Fc fusions, in response to two injections of KLH without adjuvant (on Study Day 0 and Day 12). These test articles were compared to administration of molar-matched levels of an Fc isotype control protein. Activity of test articles observed in the mouse KLH model can often predict their immunomodulatory effects in humans.

To begin the KLH study, 10-week old female C57/BL6NJ mice (The Jackson Laboratories, Sacramento, CA) were randomized into 12 groups of 5 mice each. Mice were administered 0.25 mg KLH (EMD Millipore, Cat. 374825-25MG) via intraperitoneal (IP) injection on Days 0 and 12; the original commercial stock solution of KLH was diluted to the appropriate concentration with Dulbecco's phosphate-buffered saline (DPBS) prior to injection. Mice were dosed with the test articles as outlined in Table E3 via IP injection (dosed on Days 4 and 11). The dose of test articles was molar matched to 15 mg/kg TACI-Fc. Six mice remained untreated/uninjected as naïve controls (Group 13). Serum was collected on Day 5 (24 hr post-1^{st} dose), Day 12 (24 hr post-2^{nd} dose/pre-KLH boost), and Day 20 to evaluate drug exposure, ADA, and/or anti-KLH antibody levels. One animal in Group 9 received an incomplete dose of test article and was therefore removed from the study.

TABLE E3
Test Article Descriptions and Dose Regimen

| | | | Dose | | | |
|---|---|---|---|---|---|---|
| Group | # of | | Level | | Dose Schedule (D = | Route of |
| # | Mice | Test Article(s) | (mg/dose) | (mg/kg) | Study Day) | Delivery |
| 1 | 5 | Fc control | 0.225 | 11.3 | D 4 and D 11 | IP |
| 5 | 5 | TACI 30-110 - Fc | 0.306 | 15.3 | D 4 and D 11 | IP |
| 6 | 5 | TACI 13-118 - Fc | 0.327 | 16.4 | D 4 and D 11 | IP |
| 7 | 5 | 26 TACI CRD2-Fc | 0.271 | 13.6 | D 4 and D 11 | IP |
| 8 | 5 | 27 TACI CRD2-Fc | 0.271 | 13.6 | D 4 and D 11 | IP |
| 9 | 5 | 29 TACI CRD2-Fc | 0.272 | 13.6 | D 4 and D 11 | IP |
| 13 | 6 | None (naïve) | N/A | N/A | N/A | N/A |

N/A = not applicable

On Day 20, all mice were anesthetized with isoflurane and blood collected into serum separator tubes. Mice were sacrificed, and their spleens removed, weighed, and placed into DPBS on ice. Whole blood was centrifuged, and the serum removed and stored at −80° C. until analyzed for anti-KLH levels by enzyme-linked immunosorbent assay (ELISA). Spleens were processed to single cell suspensions, the red blood cells (RBC) lysed using RBC Lysis Buffer (Biolegend, Cat. 420301) according to the manufacturer's instructions, and the cells counted in each sample using dual-fluorescence viability, using acridine orange/propidium iodide (AO/PI) staining (Nexcelom, Cat. CS2-0106-5 mL).

Each spleen sample was then stained for flow cytometry analysis of immune cell subsets using the following method: 1×10⁶ live cells were placed into a well of two 96-well plates (Corning, Cat. 3797; one plate for a B cell-specific panel and one for a T cell-specific panel), centrifuged at 1500×g for 10 seconds, the supernatant removed, and the cell pellet washed twice with DPBS. The pellets were resuspended in 100 μL of live-dead stain (LIVE/DEAD Fixable Aqua Dead Cell Stain Kit, Life Technologies Corp., 1:1000 dilution in DPBS) and incubated for 10 min in the dark at room temperature. Following two washes with flow cytometry buffer (175 μL each), tumor pellets were resuspended in Mouse BD Fc Block (diluted 1:50 with flow buffer), and incubated in the dark for an additional 5 min at RT. Without any additional washes, 50 μL of a cocktail of the following flow cytometry antibodies (diluted in flow cytometry buffer) were added to each well of cells for the B or T cell panels. For the B cell panel, the following antibodies were combined for the cocktail: anti-mouse CD19 BUV395 (clone 1D3, Becton-Dickinson; 1:100 final concentration), anti-mouse CD138 BUV737 (clone 281-2, BD Biosciences; 1:100 final concentration), anti-mouse CD138 BV421 (clone 281-2, BioLegend Inc.; 1:100, final concentration), anti-mouse CD3E BV510 (clone 17A2, BioLegend Inc.; 1:100, final concentration), anti-mouse CD45 BV510 (clone 30-F11, BioLegend, 1:100 final concentration), anti-mouse IgD BV605 (clone 11-26c.2a, BioLegend Inc.; 1:100, final concentration), anti-mouse B220 BV785 (clone RA3-6B2, BioLegend Inc.; 1:100, final concentration), anti-mouse CD95 FITC (clone SA367H8, BioLegend Inc.; 1:100, final concentration), anti-mouse CD23 PerCP Cy5.5 (clone B3B4, BioLegend Inc.; 1:100, final concentration), anti-mouse GL7 PE (clone GL7, BioLegend Inc.; 1:100, final concentration), anti-mouse Gr1 PE Cy7 (clone RB6-8C5, BioLegend Inc.; 1:100, final concentration), anti-mouse CD21 APC (clone 7E9, BioLegend Inc.; 1:100, final concentration), and anti-mouse IgM APC Cy7 (clone RMM-1, BioLegend Inc.; 1:100, final concentration). For the T cell panel, the following antibodies were combined for the cocktail: anti-mouse CD3 BUV395 (clone 145-2C11, BD Biosciences; 1:100 final concentration), anti-rat CD8 BUV737 (clone 56-6.7, BD Biosciences; 1:100 final concentration), anti-mouse PD-1 BV421 (clone 29F.1A12, BioLegend Inc.; 1:100, final concentration), anti-mouse CD11b BV510 (clone M1/70, BioLegend Inc.; 1:100, final concentration), anti-mouse CD45 BV510 (clone 30-F11, BioLegend, 1:100 final concentration), anti-mouse CD3R BV605 (clone 145-2C11, BioLegend Inc.; 1:100, final concentration), anti-mouse CD8 BV785 (clone 53-6.7, BioLegend Inc.; 1:100, final concentration), anti-mouse CD44 FITC (clone IM7, BioLegend Inc.; 1:100, final concentration), anti-mouse CD4 PerCP Cy5.5 (clone GK1.5, BioLegend Inc.; 1:100, final concentration), anti-mouse CD62L PE (clone MEL-14, BioLegend Inc.; 1:100, final concentration), anti-mouse CXCR5 PE Dazzle (clone L138D7, BioLegend Inc.; 1:100, final concentration), anti-mouse CD25 PE Cy7 (clone PC61.5, BioLegend Inc.; 1:100, final concentration), anti-mouse CD45 AF700 (clone 30-F11, BioLegend Inc.; 1:100, final concentration) and anti-B220 APC/Cy7 (clone RA3-6B2, BioLegend, 1:100 final concentration). The cells were incubated with one of the antibody cocktails in the dark, on ice, with gentle mixing for 45 min, followed by two washes with flow cytometry buffer (175 μL per wash). Cell pellets were resuspended in 200 L flow cytometry buffer and collected on an LSRII flow cytometer. Data were analyzed using FlowJo software version 10.2 (FlowJo LLC, USA) and graphed using GraphPad Prism software (Version 8.1.2). Key cellular subset identification analysis included: total B cells (B220⁺ cells), marginal zone (MZ) B cells (B220⁺, CD19⁺, CD23⁻, CD21^{high}, IgM^{high} cells), germinal center (GC) B cells (B220⁺, CD19⁺, GL7⁺, CD95⁺ cells), T follicular helper (Tfh) cells (CD45⁺, CD3⁺, CD4⁺, PD-1⁺, CD185⁺ cells), CD4⁺ T effector memory (Tₑₘ) cells (CD45⁺, CD3⁺, CD4⁺, CD44⁺, CD62L⁻ cells), and CD8⁺ Tem cells (CD45⁺, CD3⁺, CD8⁺, CD44⁺, CD62L⁻ cells).

Statistically significant differences (p<0.05) between groups were determined by one-way analysis of variance (ANOVA) and uncorrected Fisher's Least Significant Difference (LSD) multiple comparison test using GraphPad Prism software (Version 8.1.2).

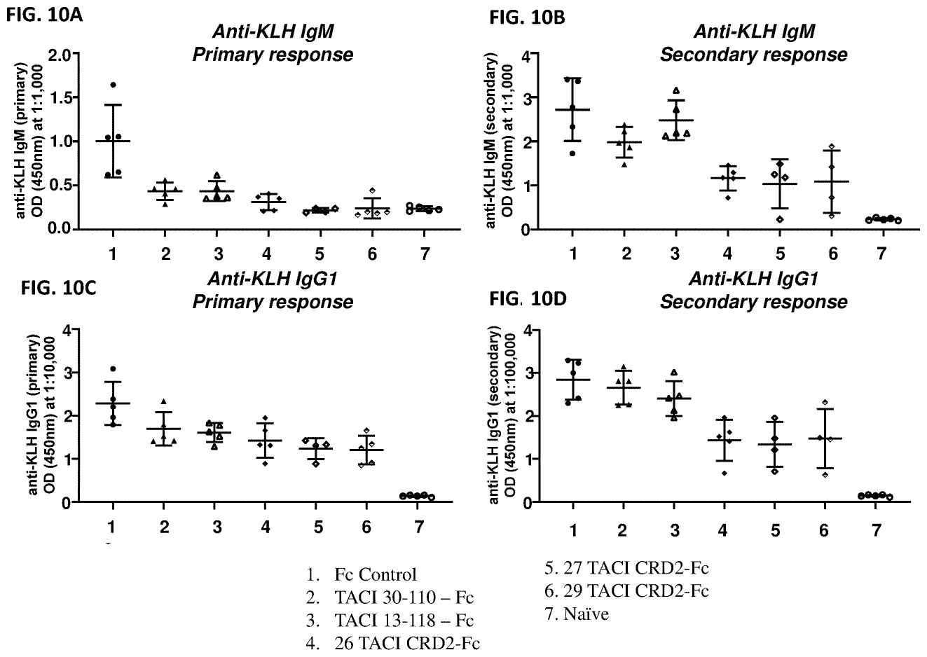

To determine the extent to which the test articles inhibited KLH-mediated antibody immune responses compared to an Fc isotype control (SEQ ID NO:73), serum samples were evaluated for concentrations of anti-KLH antibodies in two ELISA assays. The ELISA assays measured either IgM- or IgG1-specific anti-KLH levels in the serum. Mouse serum samples at numerous dilutions were incubated in plates coated with KLH, followed by washes and detection with 1:2000 goat anti-mouse IgG1:HRP or 1:5000 goat anti-mouse IgM:HRP. Color development was achieved using a TMB Substrate Kit (SeraCare) and the ELISA plates analyzed on a plate reader (SpectraMax® iD3 Microplate Reader, Molecular Devices LLC). There was no standard curve for the assay, thus optical density (OD) was used to compare the levels of anti-KLH antibodies; the higher the OD, the greater the levels of anti-KLH antibodies in the serum sample. For anti-KLH IgM OD levels, data are presented in FIG. 10A (primary response), FIG. 10B (secondary response) and statistical analysis by 1-way ANOVA and uncorrected Fisher's LSD multiple comparison test presented in Table E4 and Table E5, respectively. Anti-KLH IgG1 OD levels are presented in FIG. 10C (primary response), FIG. 10D (secondary response) and statistical analysis by 1-way ANOVA and uncorrected Fisher's LSD multiple comparison test presented in Table E6 and Table E7. Results demonstrate that each of the test articles were able to significantly reduce anti-KLH IgM levels in serum during the primary immune response compared to Fc control treatment, with 29 TACI-CRD2-Fc (SEQ ID NO: 29) demonstrating the largest reductions amongst all test articles, and TACI 30-110-Fc and TACI 13-118-Fc treatment having the most modest effect (FIG. 10A). For the secondary response on Day 20, measured 9 days after the 2^{nd} and last dose of test article, all test articles except TACI 13-118-Fc induced significant reductions in anti-KLH IgM levels, with all test articles except TACI 30-110-Fc, TACI 13-118-Fc demonstrating reduction (FIG. 10B). Each of the test articles were also able to significantly reduce anti-KLH IgG1 levels during the primary immune response compared to Fc control, with all test articles except TACI 30-110-Fc, TACI 13-118-Fc again demonstrating the greatest reductions (FIG. 10C). For the secondary response to KLH, all test articles except TACI 30-110-Fc, TACI 13-118-Fc, significantly reduced levels of anti KLH IgG1 (FIG. 10D). These results indicate that most of the molecules containing the TACI vTD were efficacious at reducing the T cell-dependent antibody immune response to KLH, with 26 TACI CRD2-Fc, 27 TACI CRD2-Fc, and 29 TACI CRD2-Fc, exhibiting the most significant effects in this mouse immunization model.

TABLE E4
Statistical Analysis of anti-KLH IgM OD
levels (primary response; FIG. 10A)

| Comparison | p-value | Significant? |
|---|---|---|
| Fc Control vs. TACI 30-110 - Fc | <0.0001 | Yes |
| Fc Control vs. TACI 13-118 - Fc | <0.0001 | Yes |
| Fc Control vs. 26 TACI CRD2-Fc | <0.0001 | Yes |
| Fc Control vs. 27 TACI CRD2-Fc | <0.0001 | Yes |
| Fc Control vs. 29 TACI CRD2-Fc | <0.0001 | Yes |
| Fc Control vs. Naive | <0.0001 | Yes |

TABLE E5
Statistical Analysis of anti-KLH IgM OD
levels (secondary response; FIG. 10B)

| Comparison | p-value | Significant? |
|---|---|---|
| Fc Control vs. TACI 30-110 - Fc | 0.0283 | Yes |
| Fc Control vs. TACI 13-118 - Fc | 0.4653 | No |
| Fc Control vs. 26 TACI CRD2-Fc | <0.0001 | Yes |
| Fc Control vs. 27 TACI CRD2-Fc | <0.0001 | Yes |
| Fc Control vs. 29 TACI CRD2-Fc | <0.0001 | Yes |
| Fc Control vs. Naive | <0.0001 | Yes |

TABLE E6
Statistical Analysis of anti-KLH IgG1
OD levels (primary response; FIG. 10C)

| Comparison | p-value | Significant? |
|---|---|---|
| Fc Control vs. TACI 30-110 - Fc | 0.0218 | Yes |
| Fc Control vs. TACI 13-118 - Fc | 0.0093 | Yes |
| Fc Control vs. 26 TACI CRD2-Fc | 0.0012 | Yes |
| Fc Control vs. 27 TACI CRD2-Fc | 0.0002 | Yes |
| Fc Control vs. 29 TACI CRD2-Fc | <0.0001 | Yes |
| Fc Control vs. Naive | <0.0001 | Yes |

TABLE E7
Statistical Analysis of anti-KLH IgG1 OD
levels (secondary response; FIG. 10D)

| Comparison | p-value | Significant? |
|---|---|---|
| Fc Control vs. TACI 30-110 - Fc | 0.5367 | No |
| Fc Control vs. TACI 13-118 - Fc | 0.1477 | No |
| Fc Control vs. 26 TACI CRD2-Fc | <0.0001 | Yes |
| Fc Control vs. 27 TACI CRD2-Fc | <0.0001 | Yes |
| Fc Control vs. 29 TACI CRD2-Fc | <0.0001 | Yes |
| Fc Control vs. Naive | <0.0001 | Yes |

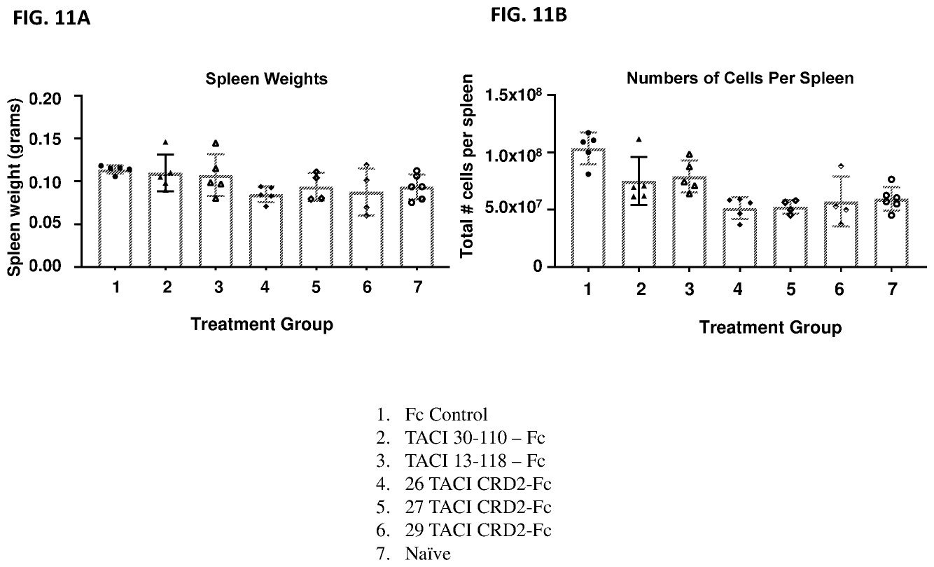

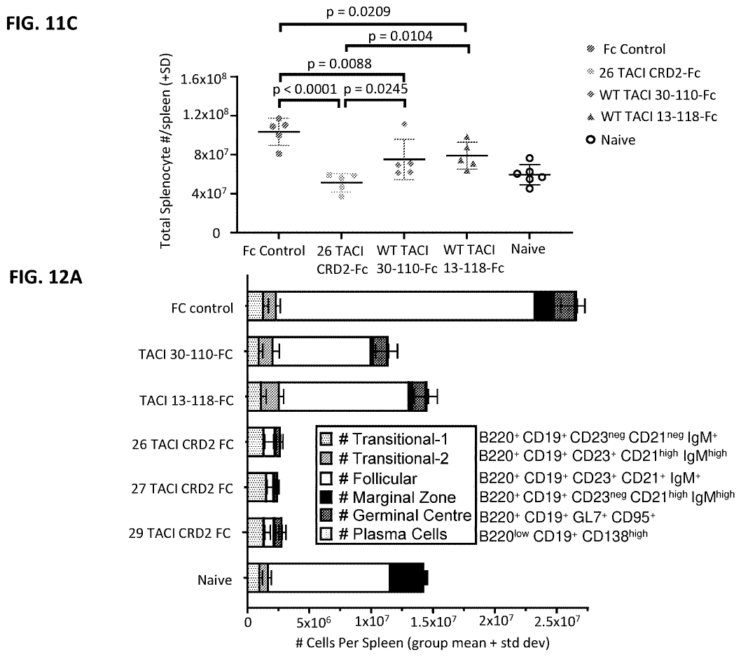

KLH immunization increased spleen size and cellularity as shown by the significant increase in splenocyte numbers in the Fc control-treated group compared to the naïve group (FIG. 11C). As shown in FIGS. 11A and 11B, mice treated with all test articles except TACI 30-110-Fc or TACI 13-118-Fc had significantly smaller spleens as assessed by weight and cell number, respectively, at the end of the study (Day 20) compared to Fc control-treated mice (Table E8). Mice treated with each of the test articles also had significantly fewer spleen cells than the Fc control group. The smaller spleens are indicative of reductions in lymphocytes, which can have immunomodulatory effects on the pathogenesis of autoimmune and inflammatory diseases associated with heightened immune responses, particularly those driven by B and/or T cells. Statistical analyses of spleen weights and total cell numbers are shown in Table E8 and Table E9, respectively.

TABLE E8
Statistical Comparisons Across All Treatment Groups for Spleen Weights (FIG. 11A):

| Treatment | Fc | TACI | TACI | 541 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI | ns | | | | | |
| 30-110-Fc | | | | | | |
| TACI | ns | ns | | | | |
| 13-118-Fc | | | | | | |
| 26 TACI | 0.0062 | 0.0172 | 0.0319 | | | |
| CRD2-Fc | | | | | | |
| 27 TACI | 0.0097 | 0.0261 | 0.0469 | ns | | |
| CRD2-Fc | | | | | | |
| 29 TACI | 0.0181 | 0.0435 | ns | ns | ns | |
| CRD2-Fc | | | | | | |
| Naive | 0.041 | ns | ns | ns | ns | ns |

TABLE E9
Statistical Comparisons Across All Treatment Groups for Splenic Cell Numbers (FIG. 11B)

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI | 0.0022 | | | | | |
| 30-110-Fc | | | | | | |
| TACI | 0.0079 | ns | | | | |
| 13-118-Fc | | | | | | |
| 26 TACI | <0.0001 | 0.0099 | 0.0029 | | | |
| CRD2-Fc | | | | | | |
| 27 TACI | <0.0001 | 0.004 | 0.0011 | ns | | |
| CRD2-Fc | | | | | | |
| 29 TACI | <0.0001 | ns | 0.0227 | ns | ns | |
| CRD2-Fc | | | | | | |
| Naive | <0.0001 | ns | 0.0241 | ns | ns | ns |

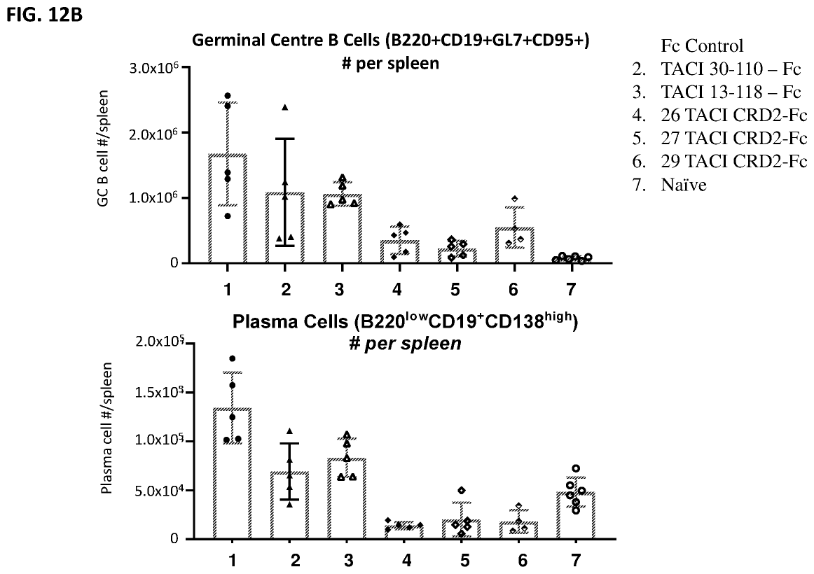

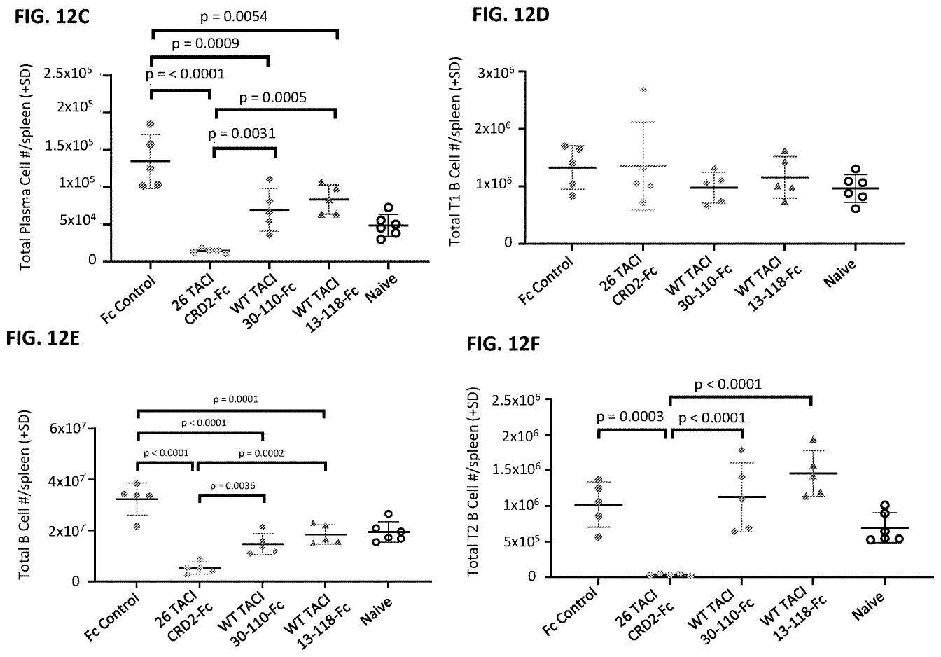

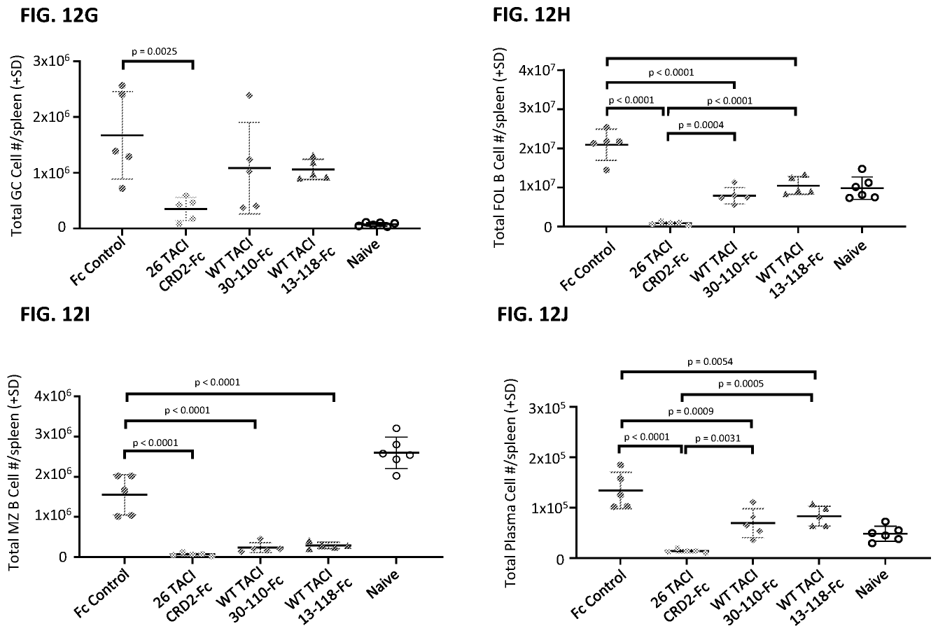

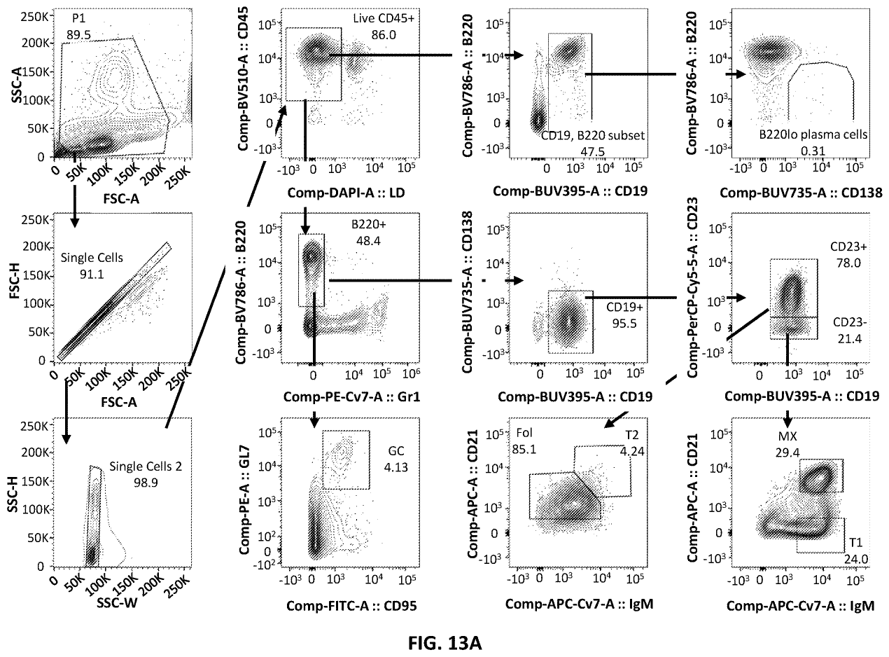

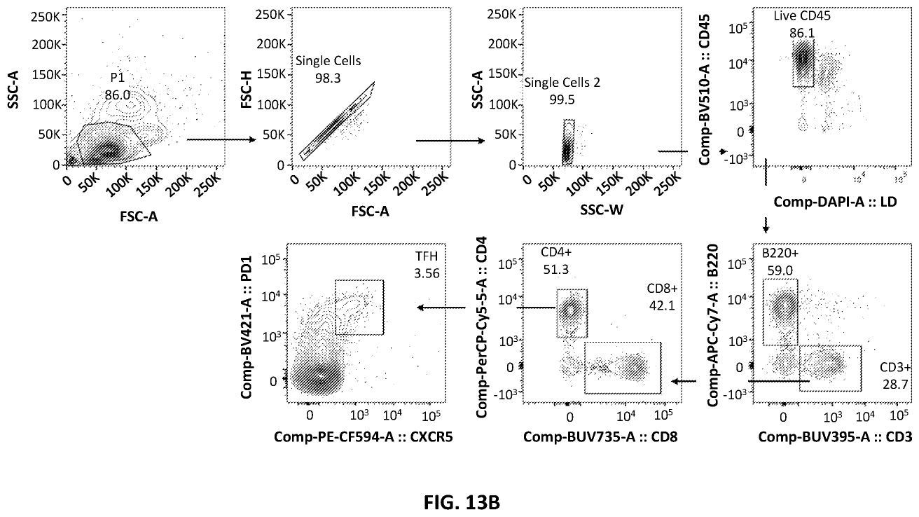

Of particular importance to the pathogenesis of autoimmune and inflammatory diseases are cell types that promote B cell survival and differentiation, antibody production, and T cell effector memory. These cell types include, but are not limited to, the following: total B cells, marginal zone (MZ) B cells, germinal center (GC) B cells, T follicular helper (Tfh) cells, and CD4⁺ and CD8⁺ T effector memory (Tₑm) cells. Therapeutics whose mechanisms of action include reducing these cell types would be anticipated to be efficacious in the treatment of numerous autoantibody-mediated diseases. Treatment with any of the TACI vTD-Fc test articles substantially reduced the numbers of multiple splenic B cell subsets compared to the remaining treatment groups, including impacts on transitional-2 (B220⁺ CD19⁺ CD23⁺ CD21^{high} IgM^{high}) follicular (B220⁺ CD19⁺ CD23⁺ CD21⁺ IgM⁺), marginal zone (B220⁺ CD19⁺ CD23^{neg} CD21^{high}IgM^{high}), germinal centre (B220⁺ CD19⁺ GL7⁺ CD95⁺), and plasma cells (B220^{low} CD19+CD138^{high}) (FIG. 12A and FIG. 12B). Except for the least mature transitional type-1 (T1) B cells, all splenic B cell subsets evaluated (gated as defined in FIGS. 13A and 13B), including transitional type 2 (T2), follicular, marginal zone (MZ), germinal center (GC), and plasma cells were significantly reduced following 26 TACI CRD2-Fc treatment, exceeding the activity of WT TACI-Fc (FIGS. 12C, FIGS. 12D-12J).

These TACI vTD-molecules were as effective or better than the two WT TACI-Fc molecules (TACI 13-188-Fc and TACI 30-110-Fc) in their ability to reduce the percentage (not shown) or numbers of these populations that are important in B cell survival and differentiation and antibody production. Statistical analyses from flow cytometry data of Day 20 splenocytes are shown in Tables E10-E28.

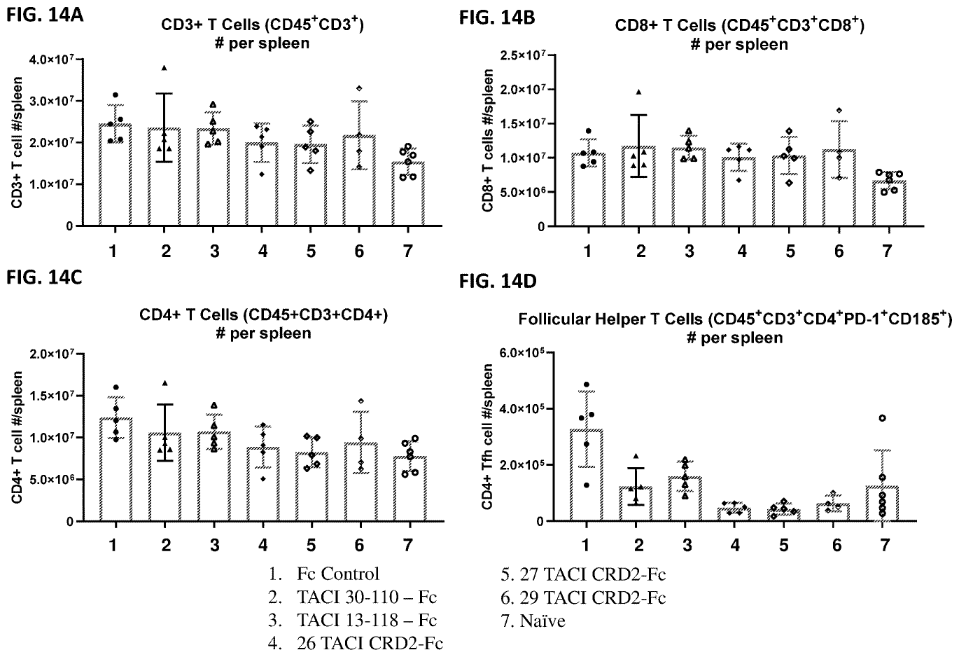

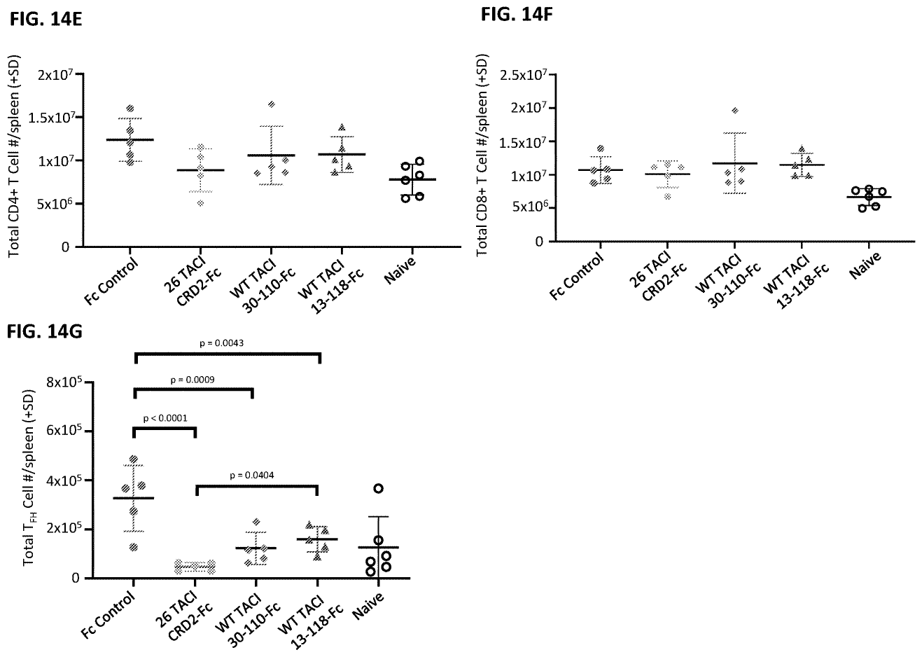

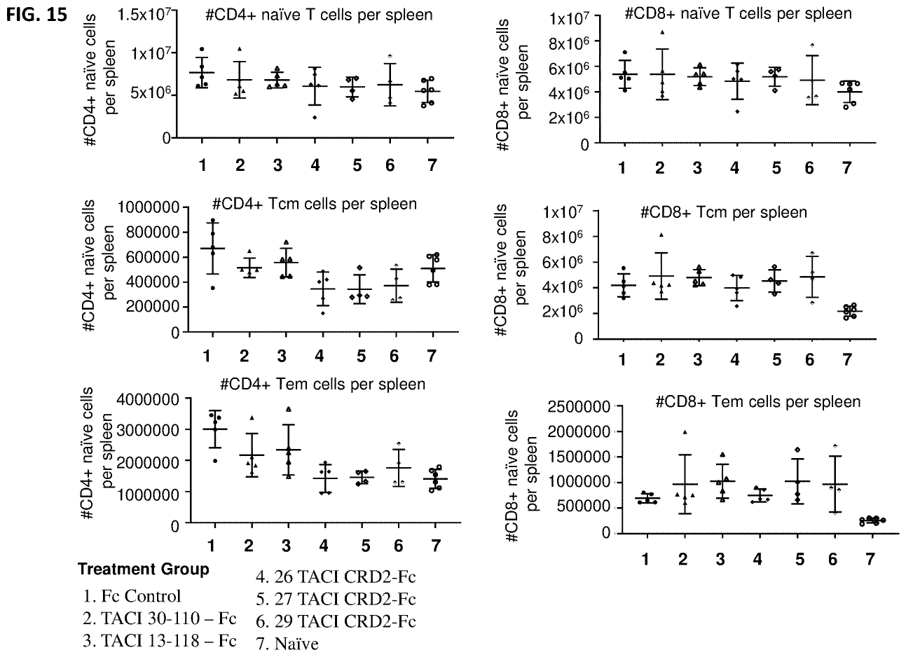

The splenic CD3⁺, CD4⁺, or CD8⁺ T cell populations were largely unaffected by the 6 TACI vTD−-containing test articles. Compared to the Fc control group (FIG. 14A-C, see also FIGS. 14E and 14F), and Tcm and Ten memory T cells compared to the Fc control group, were unaffected (FIG. 15). As compared to the Fc control, all of the test articles reduced the numbers of follicular helper T cells (CD45⁺, CD3⁺, CD4⁺, PD-1⁺, CD185⁺), which interact with B cells in the germinal center and are important contributors to T cell-dependent antibody responses (FIG. 14D, see also FIG. 14G).

TABLE E10
Statistical Analysis of Splenic B Cell Subsets-Cell Numbers vs. Fc Control Group (FIG. 12)

| | | | | Marginal | | |
|---|---|---|---|---|---|---|
| | T1 B | T2 B | Follic B | Zone B | GC B | Plasma |
| Comparison | cells | cells | cells | cells | cells | Cells |
| Fc Control vs. | 0.2738 | 0.4820 | <0.0001 | <0.0001 | 0.0152 | <0.0001 |
| TACI 30-110-Fc | | | | | | |
| Fc Control vs. | 0.5942 | 0.0045 | <0.0001 | <0.0001 | 0.0115 | 0.0012 |
| TACI 13-118-Fc | | | | | | |
| Fc Control vs. 26 | 0.9402 | <0.0001 | <0.0001 | <0.0001 | <0.0001 | <0.0001 |
| TACI CRD2-Fc | | | | | | |
| Fc Control vs. 27 | 0.4679 | <0.0001 | <0.0001 | <0.0001 | <0.0001 | <0.0001 |
| TACI CRD2-Fc | | | | | | |
| Fc Control vs. 29 | 0.9061 | <0.0001 | <0.0001 | <0.0001 | <0.0001 | <0.0001 |
| TACI CRD2-Fc | | | | | | |
| Fc Control vs. Naive | 0.2333 | 0.0241 | | <0.0001 | <0.0001 | <0.0001 |

TABLE E11
Statistical Analysis of Splenic T Cell Subsets-
Cell Numbers vs. Fc Control Group (FIG. 14A-14D)

| | | | | CD4+ |
|---|---|---|---|---|
| | CD3+ T | CD8+ T | CD4+ T | Tfh |
| Comparison | cells | cells | cells | cells |
| Fc Control vs. TACI 30-110 - Fc | 0.7623 | 0.5177 | 0.2474 | <0.0001 |
| Fc Control vs. TACI 13-118 - Fc | 0.7210 | 0.6151 | 0.2739 | 0.0001 |
| Fc Control vs. 26 TACI CRD2-Fc | 0.1513 | 0.6863 | 0.0261 | <0.0001 |
| Fc Control vs. 27 TACI CRD2-Fc | 0.1209 | 0.8049 | 0.0095 | <0.0001 |
| Fc Control vs. 29 TACI CRD2-Fc | 0.4042 | 0.7596 | 0.0728 | <0.0001 |
| Fc Control vs. Naive | 0.0038 | 0.0086 | 0.0029 | <0.0001 |

TABLE E12
Statistical Analysis of Splenic T Cell Subsets-Cell Numbers vs. Fc Control Group (FIG. 15)

| | Naïve | CD4+ | CD4+ | Naive | CD8+ | CD8+ |
|---|---|---|---|---|---|---|
| | CD4+ | Tcm | Tem | CD8+ T | Tem | Tem |
| Comparison | T cells | cells | cells | cells | cells | cells |
| Fc Control vs. | 0.4484 | 0.0695 | 0.0088 | 0.9952 | 0.2531 | 0.1411 |
| TACI 30-110-Fc | | | | | | |
| Fc Control vs. | 0.4336 | 0.1831 | 0.0355 | 0.8153 | 0.3456 | 0.0729 |
| TACI 13-118-Fc | | | | | | |
| Fc Control vs. 26 | 0.1548 | 0.0003 | <0.0001 | 0.5016 | 0.7516 | 0.7624 |
| TACI CRD2-Fc | | | | | | |
| Fc Control vs. 27 | 0.0824 | <0.0001 | <0.0001 | 0.5055 | 0.8187 | 0.2444 |
| TACI CRD2-Fc | | | | | | |
| Fc Control vs. 29 | 0.2292 | 0.0015 | 0.0004 | 0.5929 | 0.3311 | 0.1632 |
| TACI CRD2-Fc | | | | | | |
| Fc Control vs. Naive | 0.0433 | 0.0516 | <0.0001 | 0.0782 | 0.0016 | 0.0166 |

TABLE E13
Statistical Comparisons Across All Treatment
Groups for Numbers of T1 B Cells

| Treat- | | TACI | TACI | 26 TACI | 27 TACI | 29 TACI |
|---|---|---|---|---|---|---|
| ment | Fc | 30-110- | 13-118- | CRD2- | CRD2- | CRD2- |
| Group | control | Fc | Fc | Fc | Fc | Fc |
| TACI | ns | | | | | |
| 30-110- | | | | | | |
| Fc | | | | | | |
| TACI | ns | ns | | | | |
| 13-118- | | | | | | |
| Fc | | | | | | |
| 26 TACI | ns | ns | ns | | | |
| CRD2- | | | | | | |
| Fc | | | | | | |
| 27 TACI | ns | ns | ns | ns | | |
| CRD2- | | | | | | |
| Fc | | | | | | |
| 29 TACI | ns | ns | ns | ns | ns | |
| CRD2- | | | | | | |
| Fc | | | | | | |
| Naive | ns | ns | ns | ns | ns | ns |

TABLE E14
Statistical Comparisons Across All Treatment Groups for Numbers of T2 B cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI | ns | | | | | |
| 30-110-Fc | | | | | | |
| TACI | 0.0042 | 0.0268 | | | | |
| 13-118-Fc | | | | | | |
| 26 TACI | <0.0001 | <0.0001 | <0.0001 | | | |
| CRD2-Fc | | | | | | |
| 27 TACI | <0.0001 | <0.0001 | <0.0001 | ns | | |
| CRD2-Fc | | | | | | |
| 29 TACI | <0.0001 | <0.0001 | <0.0001 | ns | ns | |
| CRD2-Fc | | | | | | |
| Naive | 0.0231 | 0.0033 | <0.0001 | <0.0001 | <0.0001 | <0.0001 |

TABLE E15
Statistical Comparisons Across All Treatment Groups for Numbers of Follicular B Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI | <0.0001 | | | | | |
| 30-110-Fc | | | | | | |
| TACI | <0.0001 | ns | | | | |
| 13-118-Fc | | | | | | |
| 26 TACI | <0.0001 | <0.0001 | <0.0001 | | | |
| CRD2-Fc | | | | | | |
| 27 TACI | <0.0001 | <0.0001 | <0.0001 | ns | | |
| CRD2-Fc | | | | | | |
| 29 TACI | <0.0001 | <0.0001 | <0.0001 | ns | ns | |
| CRD2-Fc | | | | | | |
| Naive | <0.0001 | ns | ns | <0.0001 | <0.0001 | <0.0001 |

TABLE E16
Statistical Comparisons Across All Treatment Groups for Numbers of Marginal Zone B Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI | <0.0001 | | | | | |
| 30-110-Fc | | | | | | |
| TACI | <0.0001 | ns | | | | |
| 13-118-Fc | | | | | | |
| 26 TACI | <0.0001 | ns | ns | | | |
| CRD2-Fc | | | | | | |
| 27 TACI | <0.0001 | ns | ns | ns | | |
| CRD2-Fc | | | | | | |
| 29 TACI | <0.0001 | ns | ns | ns | ns | |
| CRD2-Fc | | | | | | |
| Naive | <0.0001 | <0.0001 | <0.0001 | <0.0001 | <0.0001 | <0.0001 |

TABLE E17
Statistical Comparisons Across All Treatment Groups for Numbers of Germinal Centre B Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI | 0.0182 | | | | | |
| 30-110-Fc | | | | | | |
| TACI | 0.0139 | ns | | | | |
| 13-118-Fc | | | | | | |
| 26 TACI | <0.0001 | 0.0036 | 0.0049 | | | |
| CRD2-Fc | | | | | | |
| 27 TACI | <0.0001 | 0.0008 | 0.0011 | ns | | |
| CRD2-Fc | | | | | | |
| 29 TACI | <0.0001 | 0.0403 | ns | ns | ns | |
| CRD2-Fc | | | | | | |
| Naive | <0.0001 | <0.0001 | <0.0001 | ns | ns | 0.0601 |

TABLE E18
Statistical Comparisons Across All Treatment Groups for Numbers of Plasma Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI 30- | <0.0001 | | | | | |
| 110-Fc | | | | | | |
| TACI 13- | 0.0016 | ns | | | | |
| 118-Fc | | | | | | |
| 26 TACI | <0.0001 | 0.0007 | <0.0001 | | | |
| CRD2-Fc | | | | | | |
| 27 TACI | <0.0001 | 0.0024 | 0.0001 | ns | | |
| CRD2-Fc | | | | | | |
| 29 TACI | <0.0001 | 0.0028 | 0.0002 | ns | ns | |
| CRD2-Fc | | | | | | |
| Naive | <0.0001 | ns | 0.0211 | 0.0236 | ns | ns |

TABLE E19
Statistical Comparisons Across All Treatment Groups for Numbers of CD3+ T Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI 30- | ns | | | | | |
| 110-Fc | | | | | | |
| TACI 13- | ns | ns | | | | |
| 118-Fc | | | | | | |
| 26 TACI | ns | ns | ns | | | |
| CRD2-Fc | | | | | | |
| 27 TACI | ns | ns | ns | ns | | |
| CRD2-Fc | | | | | | |
| 29 TACI | ns | ns | ns | ns | ns | |
| CRD2-Fc | | | | | | |
| Naive | 0.0038 | 0.0089 | 0.0104 | ns | ns | ns |

TABLE E20
Statistical Comparisons Across All Treatment Groups for Numbers of CD4+ T Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI 30- | ns | | | | | |
| 110-Fc | | | | | | |
| TACI 13- | ns | ns | | | | |
| 118-Fc | | | | | | |
| 26 TACI | 0.0261 | ns | ns | | | |
| CRD2-Fc | | | | | | |
| 27 TACI | 0.0095 | ns | ns | ns | | |
| CRD2-Fc | | | | | | |
| 29 TACI | ns | ns | ns | ns | ns | |
| CRD2-Fc | | | | | | |
| Naive | 0.0029 | 0.062 | ns | ns | ns | ns |

TABLE E21
Statistical Comparisons Across All Treatment Groups for Numbers of CD8+ T Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI 30- | ns | | | | | |
| 110-Fc | | | | | | |
| TACI 13- | ns | ns | | | | |
| 118-Fc | | | | | | |
| 26 TACI | ns | ns | ns | | | |
| CRD2-Fc | | | | | | |
| 27 TACI | ns | ns | ns | ns | | |
| CRD2-Fc | | | | | | |
| 29 TACI | ns | ns | ns | ns | ns | |
| CRD2-Fc | | | | | | |
| Naive | 0.023 | 0.0051 | 0.0072 | ns | 0.0387 | 0.0167 |

TABLE E22
Statistical Comparisons Across All Treatment Groups for Numbers of Follicular Helper T Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI 30- | <0.0001 | | | | | |
| 110-Fc | | | | | | |
| TACI 13- | 0.0001 | ns | | | | |
| 118-Fc | | | | | | |
| 26 TACI | <0.0001 | ns | 0.0078 | | | |
| CRD2-Fc | | | | | | |
| 27 TACI | <0.0001 | ns | 0.0058 | ns | | |
| CRD2-Fc | | | | | | |
| 29 TACI | <0.0001 | ns | 0.0293 | ns | ns | |
| CRD2-Fc | | | | | | |
| Naive | <0.0001 | ns | ns | 0.0472 | 0.036 | ns |

TABLE E23
Statistical Comparisons Across All Treatment Groups for Numbers of Naïve CD4+ T Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI 30- | ns | | | | | |
| 110-Fc | | | | | | |
| TACI 13- | ns | ns | | | | |
| 118-Fc | | | | | | |
| 26 TACI | ns | ns | ns | | | |
| CRD2-Fc | | | | | | |
| 27 TACI | ns | ns | ns | ns | | |
| CRD2-Fc | | | | | | |
| 29 TACI | ns | ns | ns | ns | ns | |
| CRD2-Fc | | | | | | |
| Naive | 0.0433 | ns | ns | ns | ns | ns |

TABLE E24
Statistical Comparisons Across All Treatment Groups for Numbers of CD4+ Tcm Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI 30- | ns | | | | | |
| 110-Fc | | | | | | |
| TACI 13- | ns | ns | | | | |
| 118-Fc | | | | | | |
| 26 TACI | 0.0003 | 0.0488 | 0.0148 | | | |
| CRD2-Fc | | | | | | |
| 27 TACI | <0.0001 | 0.0206 | 0.0056 | ns | | |
| CRD2-Fc | | | | | | |
| 29 TACI | 0.0015 | ns | 0.0415 | ns | ns | |
| CRD2-Fc | | | | | | |
| Naive | ns | ns | ns | 0.0453 | 0.0183 | ns |

TABLE E25
Statistical Comparisons Across All Treatment Groups for Numbers of CD4+ Tem Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI 30- | 0.0088 | | | | | |
| 110-Fc | | | | | | |
| TACI 13- | 0.0355 | ns | | | | |
| 118-Fc | | | | | | |
| 26 TACI | <0.0001 | 0.0188 | 0.0043 | | | |
| CRD2-Fc | | | | | | |
| 27 TACI | <0.0001 | 0.0094 | 0.002 | ns | | |
| CRD2-Fc | | | | | | |
| 29 TACI | 0.0004 | ns | ns | ns | ns | |
| CRD2-Fc | | | | | | |
| Naive | <0.0001 | 0.0128 | 0.0026 | ns | ns | ns |

TABLE E26
Statistical Comparisons Across All Treatment Groups for Numbers of Naïve CD8+ T Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI 30- | ns | | | | | |
| 110-Fc | | | | | | |
| TACI 13- | ns | ns | | | | |
| 118-Fc | | | | | | |
| 26 TACI | ns | ns | ns | | | |
| CRD2-Fc | | | | | | |
| 27 TACI | ns | ns | ns | ns | | |
| CRD2-Fc | | | | | | |
| 29 TACI | ns | ns | ns | ns | ns | |
| CRD2-Fc | | | | | | |
| Naive | ns | ns | ns | ns | ns | ns |

TABLE E27
Statistical Comparisons Across All Treatment Groups for Numbers of CD8+ Tem Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI 30- | ns | | | | | |
| 110-Fc | | | | | | |
| TACI 13- | ns | ns | | | | |
| 118-Fc | | | | | | |
| 26 TACI | ns | ns | ns | | | |
| CRD2-Fc | | | | | | |
| 27 TACI | ns | ns | ns | ns | | |
| CRD2-Fc | | | | | | |
| 29 TACI | ns | ns | ns | ns | ns | |
| CRD2-Fc | | | | | | |
| Naive | 0.0016 | <0.0001 | <0.0001 | 0.0041 | 0.0032 | 0.0001 |

TABLE E28
Statistical Comparisons Across All Treatment Groups for Numbers of CD8+ Tem Cells

| Treatment | Fc | TACI | TACI | 26 TACI | 27 TACI | 29 TACI |
|---|---|---|---|---|---|---|
| Group | control | 30-110-Fc | 13-118-Fc | CRD2-Fc | CRD2-Fc | CRD2-Fc |
| TACI 30- | ns | | | | | |
| 110-Fc | | | | | | |
| TACI 13- | ns | ns | | | | |
| 118-Fc | | | | | | |
| 26 TACI | ns | ns | ns | | | |
| CRD2-Fc | | | | | | |
| 27 TACI | ns | ns | ns | ns | | |
| CRD2-Fc | | | | | | |
| 29 TACI | ns | ns | ns | ns | ns | |
| CRD2-Fc | | | | | | |
| Naive | 0.0166 | 0.0002 | <0.0001 | 0.0073 | 0.0005 | 0.0004 |

Together, these results indicate that TACI vTD-containing single domain Fc fusion molecules, that inhibit B and/or T cell activity can reduce immune responses and cell subset changes mediated by the T cell-dependent antigen KLH in vivo (i.e. anti-KLH levels in serum and changes in immune cell subsets). These results are consistent with the evaluation of the single TACI domain B cell inhibitory molecules, as clinical therapeutics in the treatment of autoimmune and inflammatory diseases in which hyperactive lymphocytes play a role.

### Example 7. Bioactivity Assessment of TACT Blockade of TACT-Mediated Stimulation by TACT-Containing Molecules

Additional TACI vTD were generated containing one or more mutations present in exemplary TACI vTDs set forth in SEQ ID NO:26 (K77E, F78Y, Y102D), SEQ ID NO:27 (Q75E, R84Q) or SEQ ID NO: 29 (K77E, A101D, Y102D). Single, double, and triple mutations containing combinations of mutations from Q75E, K77E, F78Y, R84Q, A101D and Y102D were generated. The resulting TACI vTDs were further formatted as a TACI vTD-Fc fusion protein with an Fc domain. The exemplary generated Fc fusion proteins were generated substantially as described in Example 1. Briefly, to generate recombinant immunomodulatory proteins as Fc fusion proteins, the encoding DNA was generated to encode a protein as follows: variant TACI domain followed by a linker of 7 amino acids (GSGGGGS; SEQ ID NO: 74) followed by a human IgG1 effectorless Fc sequence containing the mutations L234A, L235E and G237A, by the Eu Index numbering system for immunoglobulin proteins (SEQ ID NO:73). For comparison, the following molecules also were tested: (1) WT TACI (68-110)-Fc (TACI 68-110, SEQ ID NO: 13, TACI-Fc SEQ ID NO: 171); and (2) a TACI-Fc with exemplary mutations K77E, F78Y and Y102D introduced into the reference TACI ECD 13-118, which was fused to the exemplary Fc sequence set forth in SEQ ID NO:73; see Example 5. Additional controls included: (3) WT TACI (13-118)-Fc (TACI 13-118, SEQ ID NO:131; corresponding to the TACI ECD portion in telitacicept); (4) WT TACI (30-110)-Fc (TACI 30-110, SEQ ID NO:130; corresponding to the TACI ECD portion in atacicept, SEQ ID NO:132); (5) BAFF-R ECD and (6) belimumab.

The generated molecules were assessed for blockade of APRIL or BAFF-mediated ligand signaling via the TACI receptor in Jurkat/NF-κB/TACI cells substantially as described in Example 2. Exemplary TACI vTD-Fc molecules were titrated from 100,000-6 pM and mixed with 30 nM human APRIL or 10 nM human BAFF, 30 minutes prior to addition of Jurkat/NF-kB/TACI cells. APRIL or BAFF-mediated ligand signaling was quantitated by monitoring luciferase production in the cells.

The results are summarized as the half maximal inhibitory concentration (IC50) of exemplary tested molecules in Table E29. The percent change in IC50 compared to the reference control WT TACI (68-110)-Fc (TACI 68-110, SEQ ID NO: 13, TACI-Fc SEQ ID NO: 171) is indicated in parentheses (ΔWT). Similar to results depicted above, the wild-type minimal CRD2 WT TACI (68-110)-Fc exhibited superior blockade of APRIL and BAFF compared to other tested control molecules, including those with sequences similar to telitacicept and atacicept. As indicated, certain mutations and combinations of mutations were associated with a further substantial increase in the ability to block APRIL or BAFF mediated ligand signaling. Together, the results show the ability of TACI vTD molecules to block APRIL and BAFF TACI-mediated ligand signaling.

TABLE E29
Exemplary TACI vTD-Fc

| SEQ ID | | APRIL IC₅₀ (pM) | BAFF IC₅₀ (pM) |
|---|---|---|---|
| NO | Mutations | (Δ WT) | (Δ WT) |
| 26 | K77E, F78Y, Y102D | 9209 (0.75) | 1552 (0.84) |
| 27 | Q75E, R84Q | 11832 (0.96) | 1461 (0.79) |
| 29 | K77E, A101D, Y102D | 2914 (0.24) | 1184 (0.64) |
| 177 | Q75E | 1938 (0.16) | 1457 (0.79) |
| 32 | K77E | 159 (0.01) | 1537 (0.83) |
| 183 | F78Y | 176 (0.01) | 1638 (0.88) |
| 30 | R84Q | 566 (0.05) | 5493 (2.96) |
| 23 | A101D | 8382 (0.68) | 1827 (0.99) |
| 190 | Y102D | 11601 (0.94) | 1863 (1.00) |
| 178 | Q75E, K77E | 10709 (0.87) | 1888 (1.02) |
| 179 | Q75E, F78Y | 13431 (1.09) | 1793 (0.97) |
| 180 | Q75E, A101D | 19999 (1.62) | 2357 (1.27) |
| 181 | Q75E, Y102D | 11096 (0.90) | 2147 (1.16) |
| 191 | K77E, F78Y | 10110 (0.82) | 1966 (1.06) |
| 24 | K77E, R84Q | 4256 (0.35) | 2258 (1.22) |
| 25 | K77E, A101D | 2039 (0.17) | 1957 (1.06) |
| 192 | K77E, Y102D | 891 (0.07) | 2178 (1.17) |
| 184 | F78Y, R84Q | 2623 (0.21) | 2260 (1.22) |
| 185 | F78Y, A101D | 2015 (0.16) | 1853 (1.00) |
| 186 | F78Y, Y102D | 8492 (0.69) | 1964 (1.06) |
| 187 | R84Q, A101D | 11200 (0.91) | 2346 (1.27) |
| 188 | R84Q, Y102D | 12300 (1.00) | 1864 (1.01) |
| 189 | A101D, Y102D | 33570 (2.72) | 1953 (1.05) |
| 182 | K77E, F78Y, R84Q | 10058 (0.82) | 2206 (1.19) |
| 13 | WT TACI (68-110) (SEQ ID | 12321 (1.00) | 1854 (1.00) |
| | NO: 171) | | |
| 1 | WT TACI (13-118) Fc1.3 | — | 7905 (4.26) |
| Atacicept | — | — | 7735 (4.17) |
| Telitacicept | — | — | 9172 (4.95) |
| Telitacicept | — | — | 7297 (3.94) |
| Telitacicept+ | K77E, F78Y, Y102D | 13168 (1.07) | 1988 (1.07) |
| BAFF-R | — | — | 53226 (28.7) |
| Belimumab | — | — | 2195 (1.18) |

### Example 8. Evaluation in Sjogren's Syndrome Model in Non-Obese Diabetic Mice

This Example describes the assessment of exemplary single domain 26-TACI-vTD Fc fusion proteins (TACI vTD SEQ ID NO:26; Fc fusion SEQ ID NO: 167 in an in vivo short-term model of Sjogren's syndrome in NOD mice, including assessment of sialadenitis, serum levels of test molecules and insulitis.

The Sjogren's syndrome model was induced in female diabetes-prone NOD/ShiLtJ mice (about 6 weeks of age) by repeat dosing of an anti-mPD-L1 antibody. Specifically, 0.1 mg of anti-mPD-L1 antibody was administered by intraperitoneal injection on days 0, 2, 4, and 6. Test molecule fusion proteins were dosed on days 0, 2 and 4 according to Table E30 below.

TABLE E30
Treatment Groups and Dosing Regimens

| | | Anti-mPD- | | | mAb |
|---|---|---|---|---|---|
| | | L1 | | | Treatment |
| | | Treatment | Test Article | TA Dose | and TA |
| Group | N | (IP) | (TA) | Level (IP) | dosing Days |
| 1 | 15 | 0.1 mg | Fc control | 0.28 mg | 0, 2, 4, 6 and |
| | | | | | 0, 2 4 |
| 3 | 15 | 0.1 mg | 26-TACI- | 0.34 mg | 0, 2, 4, 6 and |
| | | | CRD2 Fc | | 0, 2 4 |
| 6 | 5 | 0 | n/a | 0 | n/a |
| (naïve) | | | | | |

Abbreviations: IP = intraperitoneal(ly);
mg = milligram;
n/a = not applicable

Blood was obtained from the tail vein of mice (2-5 μL) on days 7, 8, 9, and 10, placed on a ReliOn Prime glucose test strip, and blood glucose (mg/dL) was measured using the ReliOn Prime Glucose Test System. At Day 10 of the experiment, mice were sacrificed and serum, submandibular glands (SMG), and pancreas were collected and analyzed.

The left SMG and pancreas were removed, dissected away from adjacent lymph nodes, and placed into neutral-buffered formalin (NBF) for approximately 72 hours, followed by transfer to 70% ethanol. The fixed tissues were embedded in paraffin, sectioned, and stained on glass slides with hematoxylin and eosin (H&E).

The scoring systems used to evaluate the extent of sialadenitis was scored as per Nandula et al. 2011 (Table 6 therein; reproduced as Table E31), and insulitis per Gutierrez et al 2014 (Table 7 therein; reproduced as Table E32).

TABLE E31
Histological Scoring Used to Evaluate Sialadenitis

| Score | Criteria |
|---|---|
| 0 | No inflammatory foci |
| 1 | 1-5 foci of >50 inflammatory cells |
| 2 | >5 foci without parenchymal destruction |
| 3 | Moderate parenchymal destruction |
| 4 | Extensive parenchymal destruction |

TABLE E32
Histological Scoring Used to Evaluate Insulitis

| Score | Criteria |
|---|---|
| 0 | No insulitis |
| 1 | Peri-islet insulitis |
| 2 | Intermediate insulitis |
| 3 | Intra-islet insulitis |
| 4 | Complete islet insulitis |

Statistically significant differences between groups for histology scores were determined using Student's t-test. GraphPad PRISM® software (Version 8.1.2) was used for statistical analyses and p values <0.05 were considered statistically significant for all statistical tests.

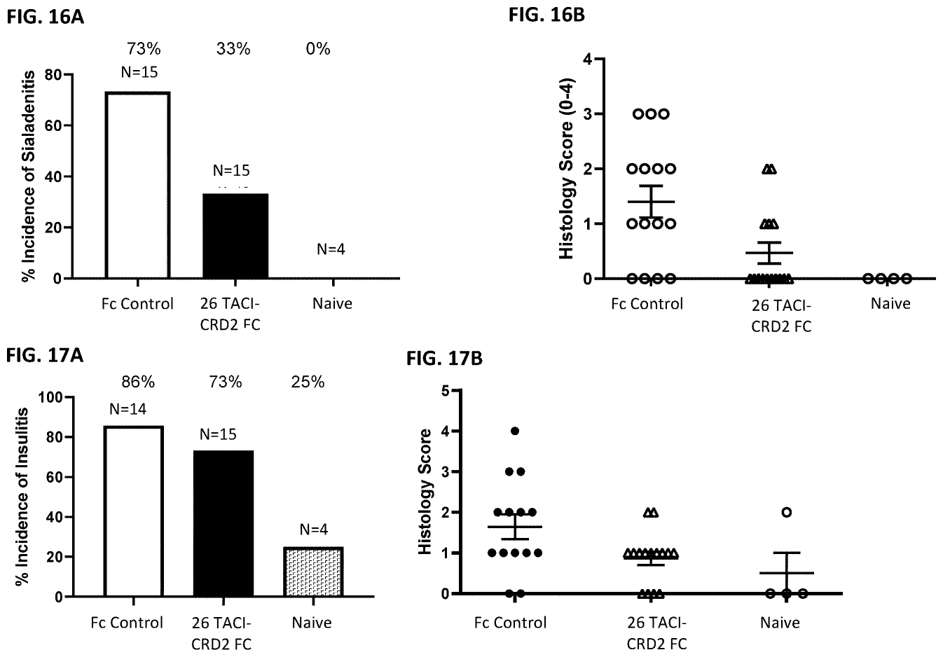

Treatment with the exemplary 26-TACI-CRD2 Fc fusion protein reduced incidence of sialadenitis (FIG. 16A) and resulted in a significantly lower histology score (p<0.01) than the mean scores for Fc control (FIG. 16B). These results are consistent with a finding that treatment of anti-PD-L1 injected NOD mice with the tested molecules reduced both the incidence and severity of sialadenitis in this model of Sjogren's syndrome.

The overall incidence of insulitis in these diabetes-prone mice and the degree of insulitis after treatment with the tested molecules is shown in FIG. 17A and FIG. 17B. 26-TACI-CRD2 Fc fusion proteins significantly reduced the degree of insulitis, as assessed by histological analysis (FIG. 17B).

Together, these results indicate treatment with the tested exemplary TACI-Fc molecule reduced the incidence and severity of sialadenitis in this mouse model of Sjogren's syndrome. These results indicate the potential for TACI molecules in therapeutic use for treating Sjogren's syndrome, and for TACI-CTLA-4 multi-domain stack molecules as therapeutics to impact the onset of type 1 diabetes in humans.

### Example 9. Assessment of Exemplary Monomeric and Tetrameric Constructs

Additional TACI-Fc fusion proteins were generated containing one (monomeric) or four (tetrameric barbell and tetrameric tandem) TACI vTD domains using the WT TACI of different lengths: 68-110 (set forth in SEQ ID NO:13), 29-110 (set forth in SEQ ID NO: 1) or 13-118 (set forth in SEQ ID NO: 131), and the TACI vTD set forth in SEQ ID NO:26 (K77E, F78Y, Y102D). The monomeric and tetrameric TACI WT and TACI vTD were formatted as TACI WT and TACI vTD-Fc fusion proteins with an Fc domain. The exemplary generated Fc fusion proteins were generated substantially as described in Example 1 and are described in Tables E33A-E33C.

Briefly, to generate recombinant monomeric immunomodulatory proteins as single chain Fc fusion proteins, the encoding DNA was generated to encode a protein as follows: WT TACI or variant TACI domain followed by a linker of 12 amino acids (GSGGGGSGGGGS; SEQ ID NO: 194) followed by a single chain Fc (scFc) set forth in SEQ ID NO: 218 (composed of a human IgG1 effectorless Fc sequence containing the mutations L234A, L235E and G237A, by the Eu Index numbering system for immunoglobulin proteins (SEQ ID NO:73), followed by a (GGGGS)₁₃ linker (SEQ ID NO:195) followed by a second human IgG1 effectorless Fc sequence containing the mutations L234A, L235E and G237A, by the Eu Index numbering system for immunoglobulin proteins). The long linker, e.g. set forth in SEQ ID NO:195, connects the C-terminus of the first Fc unity to the N-terminus of the second Fc unit forming the scFc. The generated molecules are summarized in Table E33A.

TABLE E33A
Exemplary Monomeric Immunomodulatory Proteins

| AA | NT | | TACI | LINKER | FC |
|---|---|---|---|---|---|
| SEQ | SEQ | | SEQ | SEQ | SEQ |
| ID NO | ID NO | DESCRIPTION | ID NO | ID NO | ID NO |
| 196 | 207 | TACI WT 13 | 13 | 194 | 218 |
| | | GS(G4S)2 | | | |
| | | (194) sc_Fc 218 | | | |
| 199 | 210 | TACI 26 | 26 | 194 | 218 |
| | | GS(G4S)2 | | | |
| | | (194) scFc_218 | | | |
| 203 | 214 | TACI WT 1 | 1 | 194 | 218 |
| | | GS(G4S)2 | | | |
| | | (194) scFc_218 | | | |
| 205 | 216 | TACI WT 131 | 131 | 194 | 218 |
| | | GS(G4S)2 | | | |
| | | (194) scFc_218 | | | |

To generate recombinant tetrameric immunomodulatory proteins as Fc fusion proteins, proteins were generated in different formats as follows:

In one format, the encoding DNA was generated to encode three different protein versions as follows: WT TACI (SEQ ID NO NO:198): WT TACI domain SEQ ID NO:13 followed by a linker of (G4S)4 SEQ ID NO: 84; followed by a WT TACI domain SEQ ID NO: 13; followed by a linker of GSGGGGS SEQ ID NO: 74; followed by a human IgG1 effectorless Fc sequence containing the mutations L234A, L235E and G237A, by the Eu Index numbering system for immunoglobulin proteins (SEQ ID NO:73).

In one format, the encoding DNA was generated to encode three different protein versions as follows: WT TACI (SEQ ID NO:202): WT TACI domain SEQ ID NO:13 followed by a linker of GSGGGGS SEQ ID NO: 74; followed by a human IgG1 effectorless Fc sequence containing the mutations L234A, L235E and G237A, by the Eu Index numbering system for immunoglobulin proteins (SEQ ID NO:73) followed by a linker of (G4S)4 SEQ ID NO: 84 followed by WT TACI domain SEQ ID NO:13.

In one format, the encoding DNA was generated to encode three different protein versions as follows: TACI vTD Barbell (SEQ ID NO:201): TACI vTD set forth in SEQ ID NO:26 followed by a linker of GSGGGGS SEQ ID NO: 74; followed by a human IgG1 effectorless Fc sequence containing the mutations L234A, L235E and G237A, by the Eu Index numbering system for immunoglobulin proteins (SEQ ID NO:73) followed by a linker of (G4S)4 SEQ ID NO: 84 followed by TACI vTD set forth in SEQ ID NO:26.

TABLE E33B
Exemplary Tetrameric Immunomodulatory Proteins

| AA | NT | | | | | | |
|---|---|---|---|---|---|---|---|
| SEQ | SEQ | | | | | | |
| ID | ID | | 1^{ST} | | 2^{ND} | | |
| NO | NO | DESCRIPTION | TACI | LINKER | TACI | LINKER | Fc |
| 198 | 209 | TACI WT 13 | 13 | 84 | 13 | 74 | 73 |
| | | (G4S)4 (84) | | | | | |
| | | (TACI WT 13 | | | | | |
| | | GSG4S (74) Fc | | | | | |
| | | 73 | | | | | |

TABLE E33C
Exemplary Tetrameric Immunomodulatory Proteins

| AA | NT | | | | | | |
|---|---|---|---|---|---|---|---|
| SEQ | SEQ | | | | | | |
| ID | ID | | 1^{ST} | | 2^{ND} | | |
| NO | NO | DESCRIPTION | TACI | LINKER | TACI | LINKER | Fc |
| 202 | 213 | TACI WT 13 | 13 | 74 | 73 | 84 | 13 |
| | | GSG4S (74) Fc | | | | | |
| | | 73(G4S)4 (84) | | | | | |
| | | (TACI WT 13 | | | | | |
| 201 | 212 | TACI 26 GSG4S | 26 | 74 | 73 | 84 | 26 |
| | | (74) Fc 73 | | | | | |
| | | (G4S)4 (84) | | | | | |
| | | TACI 26 | | | | | |

### A. Bioactivity of Exemplary Multi-Domain Molecules

In one experiment, exemplary molecules set forth in Tables E33A-C were assessed using the Jurkat/NF-κB/TACI reporter cells for blockade of APRIL- or BAFF-mediated signaling, substantially as described in Example 1. Activity was assessed for inhibition of the soluble BAFF (3-mer) or for inhibition of an oligomer of twenty BAFF 3-mers (BAFF 60-mer). Table E34 provides the values for half maximal inhibitory concentration (IC50) for inhibition of APRIL- and BAFF-mediated TACI signaling. In some instances, the proteins tested were not compared to their parental of WT controls and appear as (−) in the Table below. The results in Table E34 demonstrate that all generated formats block BAFF and APRIL binding.

TABLE E34
Assessment of Exemplary Monomeric and
Tetrameric Immunomodulatory Proteins

| | | | | BAFF |
|---|---|---|---|---|
| | | APRIL | BAFF | 60-mer |
| SEQ | | IC₅₀ | IC₅₀ | IC₅₀ |
| ID NO | Description | (pM) | (pM) | (pM) |
| 196 | TACI WT 13 GS(G4S)2 | — | 5695 | 24081 |
| | (194) sc_Fc 218 | | | |
| 198 | TACI WT 13 (G4S)4 (84) | 34554 | 3287 | 4333 |
| | (TACI WT 13 GSG4S | | | |
| | (74) Fc 73 | | | |
| 202 | TACI WT 13 GSG4S (74) Fc | 11910 | 1039 | 2581 |
| | 73(G4S)4 (84) (TACI WT 13 | | | |
| 199 | TACI 26 GS(G4S)2 (194) | — | 8237 | 106021 |
| | scFc_218 | | | |
| 201 | TACI 26 GSG4S (74) Fc 73 | 3762 | 779 | 778 |
| | (G4S)4 (84) TACI 26 | | | |
| 203 | TACI WT 1 GS(G4S)2 (194) | — | 4422 | 15801 |
| | scFc_218 | | | |
| 205 | TACI WT 131 GS(G4S)2 | — | 4577 | 14268 |
| | (194) scFc_218 | | | |

### Example 10. Evaluation of Exemplary TACI vTD-Fcs in a Pharmacokinetic/Pharmacodynamic Study Following a Single Intravenous Infusion in Male Sprague Dawley Rats

The Example describes the tolerability, pharmacokinetics, and pharmacodynamics of the exemplary variant fusion protein 26 TACI CRD2-Fc, generated in either a HEK-293 cell line (26 TACI CRD2-Fc (HEK-293)), or generated in a CHOZN cell line (26 TACI CRD2-Fc (CHOZN)), when administered by a single intravenous infusion to male Sprague Dawley rats.

Exemplary variant fusion proteins 26 TACI CRD2-Fc (HEK-293) and 26 TACI vTD-Fc (CHOZN), were administered to 3 male rats per group via intravenous bolus injection at 20 mg/kg once on Day 1. Dose formulations were prepared based on the analytical results from preparations used for dosing.

Endpoints assessed included clinical observations, food consumption, body weight, and serum immunoglobulins. Blood was collected at multiple time-points to characterize 26 TACI CRD2-Fc (HEK-293) and 26 TACI CRD2-Fc (CHOZN), and analyzed as serum concentrations over time. The in-life portion of this study was completed on Day 22.

Except for one animal administered 26 TACI CRD2-Fc (CHOZN), the Tₘₐₓ for both test articles was observed at 0.083 hours post-dose. Exposure, based on mean Cₘₐₓ and AUC₀₋ₜ, was also similar between the two test articles. The t_{1/2} was consistent in two animals per group (range=3.66 to 4.89 days) but variable in the third 26 TACI CRD2-Fc (HEK-293); 10.3 days, 26 TACI CRD2-Fc (CHOZN)-1.57 days). No differences in clinical observations, changes in food consumption, or changes in body weights were observed over the course of this study for 26 TACI CRD2-Fc (HEK-293) compared to 26 TACI CRD2-Fc (CHOZN) (data not shown). The test articles were administered via a bolus (rather than slow infusion) intravenous injection, a method that may account for the observed inter-animal variability.

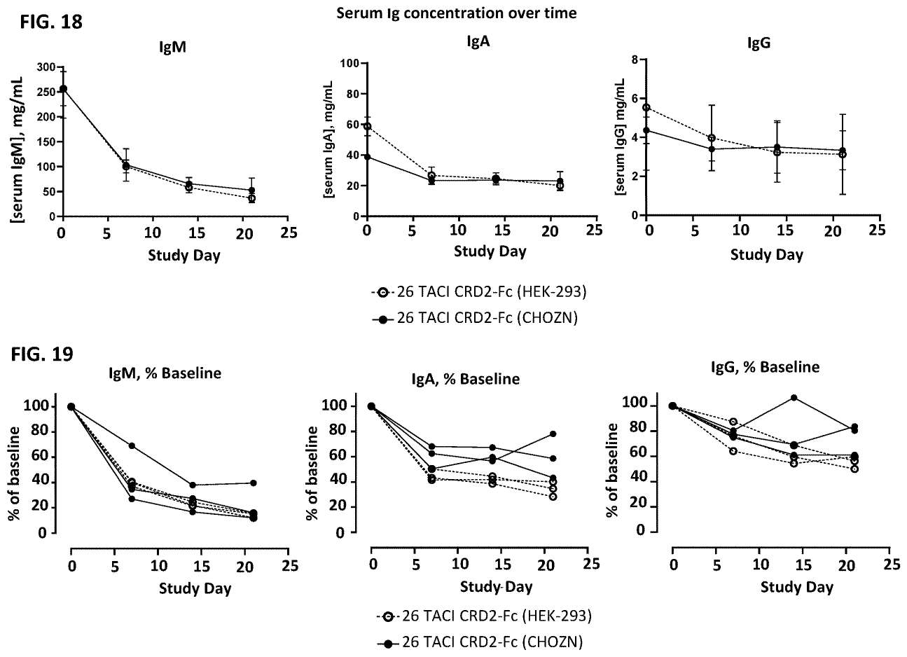

Serum immunoglobulin (IgM, IgA, and IgG) concentrations declined an average of 86%, 66%, and 45% from baseline at Day 22, respectively, for 26 TACI CRD2-Fc (HEK-293), and dropped an average of 77%, 40%, and 25% from baseline, respectively, for 26 TACI CRD2-Fc (CHOZN) (FIG. 18 and FIG. 19). Mean Cₘₐₓ was 231 and 249 μg/mL, and mean AUC₀₋ₜ was 473 and 554 day*μg/mL for 26 TACI CRD2-Fc (HEK-293) and (CHOZN), respectively.

In conclusion, 20 mg/kg 26 TACI CRD2-Fc (HEK-293) or 26 TACI CRD2-Fc (CHOZN), administered via a single intravenous bolus injection to rats resulted in good tolerability, and similar PK profiles and decreases in serum immunoglobulin levels. These results are consistent with a finding that production of the TACI-Fc fusion protein in either mammalian HEK-293 or CHO cells results in similar pharmacokinetics/pharmacodynamics.

### Example 11. Comparison of Exemplary TACI vTD-Fes to WT TACI-Fc Proteins in a Pharmacokinetic/Pharmacodynamic Study Following a Single Intravenous Infusion in Female Cynomolgus Monkeys

This Example describes the evaluation of the pharmacokinetics and pharmacodynamics of 4 exemplary variant TACI-Fc fusion proteins when administered by a single intravenous infusion over a 30-minute period to cynomolgus monkeys. The variant TACI-Fc fusion proteins in this example were generated by expression in CHOZN cells.

Female cynomolgus monkeys (2/group) were administered a single intravenous (IV) infusion over 30-minutes (±3 minutes) of vehicle buffer (25 mM Tris, 161 mM Arginine, pH 7.5) (0 mg/kg), or 9 mg/kg 26 TACI CRD2-Fc (SEQ ID NO:167), 26 TACI CRD2-Fc 81 (SEQ ID NO:168), TACI 13-118-Fc (corresponding to the TACI ECD portion in telitacicept set forth in SEQ ID NO:131 with effectorless IgG1 Fc; SEQ ID NO:241) or TACI 13-118-Fc 81 (TACI 13-118 set forth in SEQ ID NO:131 with wildtype IgG1 Fc; SEQ ID NO:240) as outlined in Table E35A below. As another comparator, results were compared to atacicept administered intravenously at 1 mg/kg from published data (Carbonatto et al. (2008) Toxicol. Sci. 105:200-210). Dose formulations were administered using a temporary catheter inserted into a peripheral vein connected to an infusion line. The appropriate volume was delivered using an infusion pump.

TACI 13-118 - Fc-81
(SEQ ID NO: 240)
SRVDQEERFPQGLWTGVAMRSCPEEQYWDPLLGTCMSCKTICNHQSQRTC
AAFCRSLSCRKEQGKFYDHLLRDCISCASICGQHPKQCAYFCENKLRSPV
NLPPELDKPHTCPLCPAPELLGGPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKATPPVLDSDGSFFLYSKLTVDK
SRWQQGNVFSCSVMHEALHNHYTQKSLSLSPGK
TACI 13-118-Fc (SEQ ID NO: 241; see also
SEQ ID NO: 3 of U.S. Pat. No. 8,193,316)
SRVDQEERFPQGLWTGVAMRSCPEEQYWDPLLGTCMSCKTICNHQSQRTC
AAFCRSLSCRKEQGKFYDHLLRDCISCASICGQHPKQCAYFCENKLRSPV
NLPPELDKTHTCPPCPAPEAEGAPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPSSIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPGK

TABLE E35A
Test Article Descriptions and Dose Regimen

| | # of | | Dose | Dose | |
|---|---|---|---|---|---|
| | Female | | Level | Volume | Route of |
| Group # | Cynos | Test Article(s) | (mg/kg) | (mL/kg) | Delivery |
| 1 | 2 | Vehicle | 0 | 2.5 | IV |
| | | (25 mM Tris, 161 mM | | | |
| | | Arginine, pH 7.5) | | | |
| 2 | 2 | 26 TACI CRD2-Fc; | 9 | 2.5 | IV |
| | | SEQ ID NO: 167 | | | |
| 3 | 2 | 26 TACI CRD2- Fc 81 | 9 | 2.5 | IV |
| | | (SEQ ID NO: 168) | | | |
| 4 | 2 | TACI 13-118 - Fc | 9 | 2.5 | IV |
| 5 | 2 | TACI 13-118 - Fc 81 | 9 | 2.5 | IV |

### Pharmacokinetic Analyses

Serum PK data were imported into Phoenix WinNonlin v8.3 (Certara, Princeton, NJ) for analysis. A standard non-compartmental model with IV infusion dosing was used to estimate the individual animal PK parameters. Nominal sample collection times relative to the start of infusion were used for the calculations. AUC values were estimated using the linear up/log down trapezoidal method.

### Serum PK

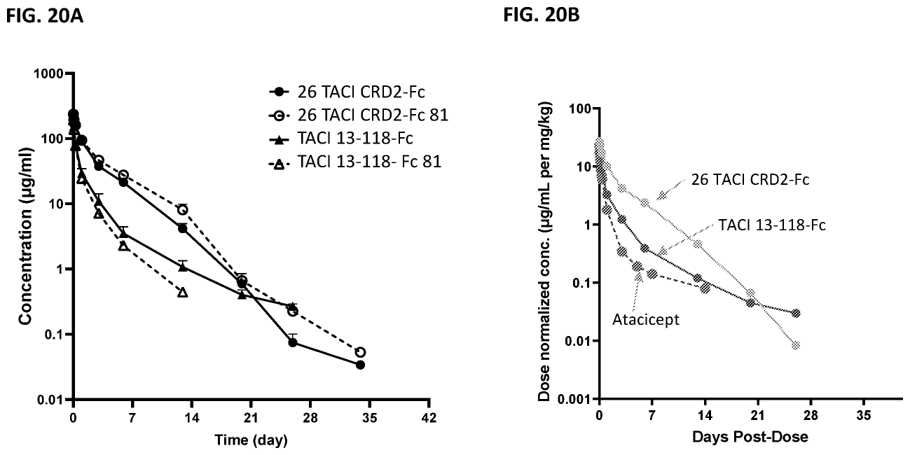

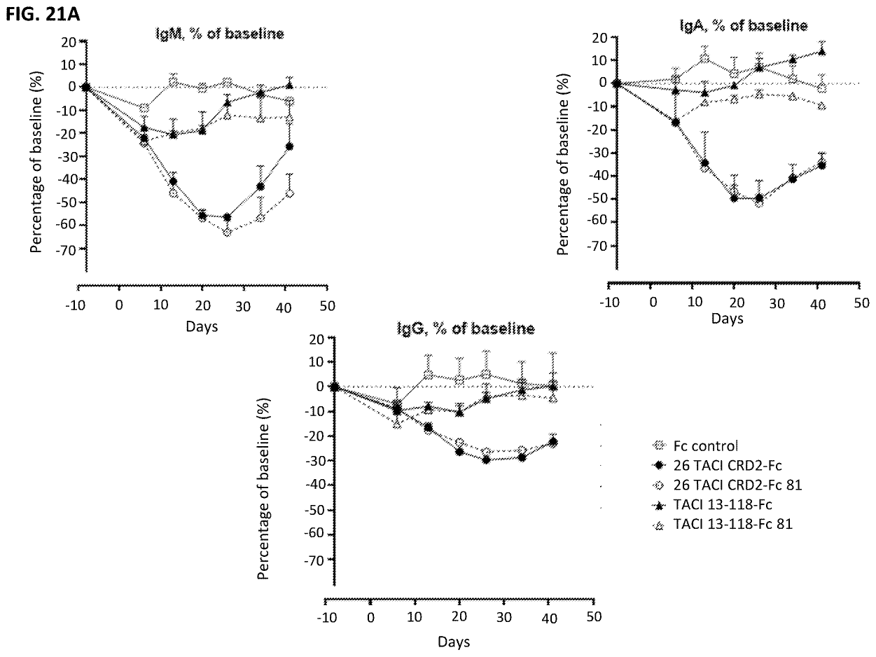

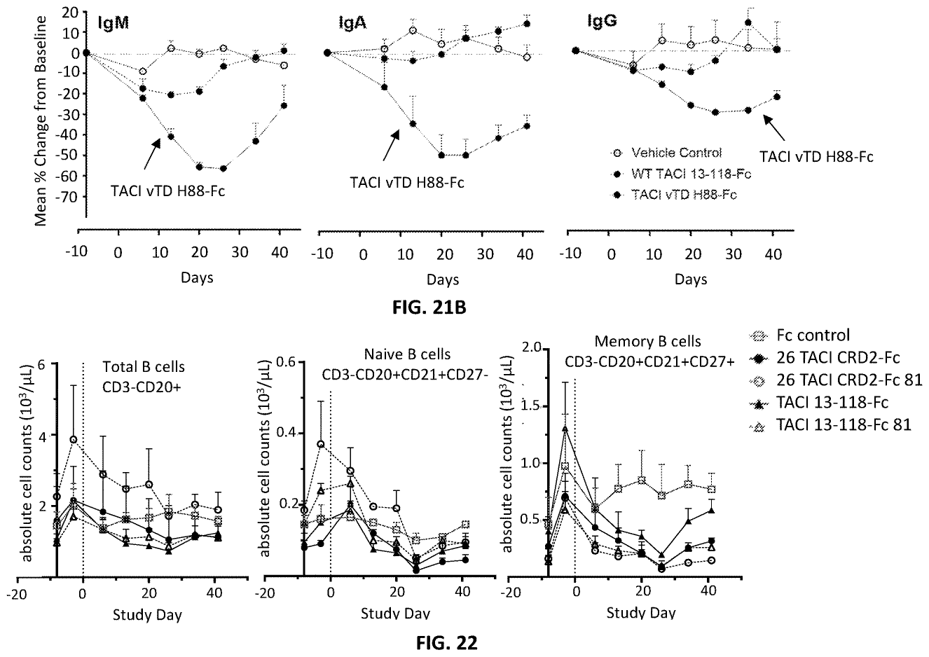

Individual animal serum concentration versus time profiles (mean+range) for each of the test articles are shown in FIG. 20. The levels of serum IgM, IgA, and IgG in animals receiving 26 TACI CRD2-Fc or 26 TACI CRD2-Fc 81 decreased an average of approximately 60%, 50%, and 30% of baseline at their nadir on Day 27, respectively (FIG. 21A, see also FIG. 21B). Similar results were observed whether the fusion construct included the effectorless Fc (SEQ ID NO: 73) or the wild-type Fc (SEQ ID NO: 81). FIG. 20B show results as further compared to Atacicept. The results in FIG. 20B are summarized in Table E35B.

TABLE E35B
Noncompartmental PK Parameters

| | Cmax/Dose | AUC/Dose |
|---|---|---|
| | (μg/mL per | (μg*hr/mL |
| | mg/kg) | per mg/kg) |
| 26 TACI CRD2-Fc | 27 | 1167 |
| WT TACI 13-118-Fc | 25 | 397 |
| WT TACI 30-110-Fc | 23 | 215* |

*Note:
WT TACI 30-110-Fc value is reported as AUC_{0-inf}, not AUC₀₋ₜ

Following a single 9 mg/kg IV dose of 26 TACI CRD2-Fc, serum concentrations were measurable out to 34 or 26 days post-dose in the two dosed animals, respectively (LLOQ=19.5 ng/mL). Following a single 9 mg/kg IV dose of 26 TACI CRD2-Fc 81, serum concentrations were measurable out to 34 or 26 days post-dose in the two dosed animals, respectively (LLOQ=39 ng/mL). Following a single 9 mg/kg IV dose of 26 TACI CRD2-Fc, serum concentrations were measurable out to 26 or 20 days post-dose in the two dosed animals, respectively (LLOQ=156 ng/mL). Following a single 9 mg/kg IV dose of TACI 13-118 Fc 81, serum concentrations were measurable out to 13 days post-dose in both dosed animals (LLOQ=156 ng/mL).

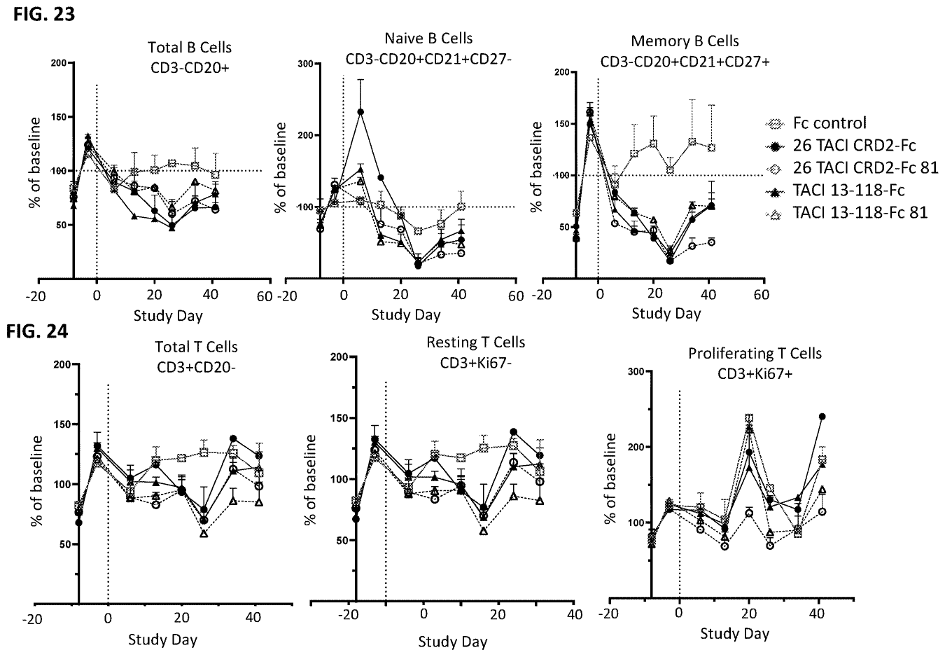

Immunophenotyping of whole blood throughout the study indicated that multiple changes were observed in lymphocyte populations following test article administration in Groups 2-5, as compared to baseline values (average of Days −8 and −3). FIG. 22 depicts absolute cell counts and FIG. 23 depicts % of cells from baseline. Despite typical inter-animal variability in absolute cell counts, there were clear reductions as compared to baseline in various B cell subsets in animals treated with 26 TACI CRD2-Fc, 26 TACI CRD2-Fc 81, TACI 13-118 Fc or TACI 13-118-Fc 81 (FIG. 22 and FIG. 23). Specifically, decreases in absolute B cell counts and % change from baseline were observed within the memory B cells (CD3-CD20+CD21+CD27+), with nadirs on Day 27 followed by a slight upward trend on Days 35 and 42 (right panels of FIGS. 22 and 23). Test article-related alterations in other B cell subsets evaluated, including naïve (CD3−CD20+CD21+CD27−) B cells were not as apparent, attributable possibly to the small group sizes and inter-animal variability.

Slight decreases in total T cells (CD3+CD20−) and resting T cells (CD3+Ki67−), were observed on Days 20 and 27 in all four test article-treated groups. No test article-related impact on absolute counts or relative percentages of the relatively infrequent proliferating T cells (CD3+Ki67+) was observed (FIG. 24).

Table E36 depicts pharmacokinetic (PK) parameters following dosing. Following IV dosing, the Tₘₐₓ for all test articles was observed at 0.0236 days post-start of infusion (i.e., 0.083 hr after the end of infusion, the first measured timepoint). Exposure based on mean Cₘₐₓ was similar (within 25%) between all four test articles. However, exposure based on AUC₀₋ₜ was approximately 3 to 4 times higher after 26 TACI CRD2-Fc and 26 TACI CRD2-Fc 81 dosing compared to the TACI 13-118-Fc or TACI 13-118-Fc 81 test articles. This difference in exposure corresponded to a lower CL and Vₛₛ in the 26 TACI CRD2-Fc and 26 TACI CRD2-Fc 81 groups compared to the TACI 13-118-Fc or TACI 13-118-Fc 81 groups (Table 3). TACI 13-118-Fc appeared to have the longest t_{1/2} (mean t_{1/2}=5.14 days compared to 2.57 to 3.47 in the other dose groups).

Anti-drug antibodies (ADA) may have affected the PK profile of 26 TACI CRD2-Fc 81, as both animals that received this test article developed relatively high titers (≥1:1000) by Day 26. Animals from all the other dose groups were either negative for ADA, or had relatively low titers (titer=1:100).

TABLE E36
Non-compartmental PK Parameters Following a Single 30-Minute
9 mg/kg IV Infusion to Female Cynomolgus Monkeys

| | | | | | | AUC₀₋ₜ | AUC_{0-∞} | | CL | V_{SS} | V_{Z} |
|---|---|---|---|---|---|---|---|---|---|---|---|
| | | | Tₘₐₓ | Cₘₐₓ | Tₗₐₛₜ | day* | day* | t_{1/2} | mL/ | mL/ | mL/ |
| | Test | | day | μg/mL | day | μg/m | μg/m | day | day/kg | kg | kg |
| Group | Article | ID | Estimate | | | | | | | | |
| 2 | 26 TACI | 1 | 0.0236 | 252 | 34.0 | 470 | 470 | 3.08 | 19.1 | 71.1 | 85.1 |
| | CRD2-Fc | 2 | 0.0236 | 231 | 26.0 | 402 | 402 | 2.12 | 22.4 | 74.2 | 68.7 |
| | (SEQ ID | Mean | 0.0236 | 242 | 30.0 | 436 | 436 | 2.60 | 20.8 | 72.6 | 76.9 |
| | NO: 167), | | | | | | | | | | |
| 3 | 26 TACI | 3 | 0.0236 | 236 | 34.0 | 487 | 487 | 4.39 | 18.5 | 72.6 | 117 |
| | CRD2-Fc | 4 | 0.0236 | 236 | 26.0 | 558 | 559 | 2.54 | 16. | 70.4 | 59.0 |
| | 81 (SEQ | mean | 0.0236 | 236 | 30.0 | 522 | 523 | 3.47 | 17.3 | 71.5 | 88.0 |
| | ID | | | | | | | | | | |
| | NO: 168) | | | | | | | | | | |
| 4 | TACI 13- | 5 | 0.0236 | 215 | 26.0 | 177 | 179 | 5.58 | 50.3 | 167 | 405 |
| | 118-Fc | 6 | 0.0236 | 230 | 20.0 | 128 | 130 | 4.70 | 69.2 | 185 | 469 |
| | (SEQ ID | mean | 0.0236 | 223 | 23.0 | 152 | 155 | 5.14 | 59.7 | 176 | 437 |
| | NO: 171) | | | | | | | | | | |
| 5 | TACI 13- | 7 | 0.0236 | 220 | 13.0 | 120 | 121 | 2.52 | 74.4 | 126 | 271 |
| | 118-Fc | 8 | 0.0236 | 167 | 13.0 | 114 | 115 | 2.61 | 78.0 | 162 | 294 |
| | 81 | mean | 0.0236 | 193 | 13.0 | 117 | 118 | 2.57 | 76.2 | 144 | 282 |

In summary, single administration of 26 TACI CRD2-Fc, 26 TACI CRD2-Fc 81, TACI 13-118-Fc or TACI 13-118-Fc 81 via 30-minute intravenous infusion to female cynomolgus monkey at 9 mg/kg resulted in higher exposure of 26 TACI CRD2-Fc and 26 TACI CRD2-Fc 81, when compared with the TACI 13-118-Fc or TACI 13-118-Fc 81 groups. Test article-related decreases in serum IgM, IgA, and IgG concentrations were most dramatic in the animals dosed with 26 TACI CRD2-Fc or 26 TACI CRD2-Fc 81, reaching their nadir between Day 21 and Day 27. Decreases in absolute counts and percent change from baseline of CD20+CD21+ B cell populations were observed in animals treated with the test articles, with the lowest levels observed at Day 27 in the animals treated with 26 TACI CRD2-Fc or 26 TACI CRD2-Fc 81. Thus, both 26 TACI CRD2-Fc and 26 TACI CRD2-Fc 81 exhibited higher overall exposures and more potent reductions in serum IgM, IgA, and IgG than either TACI 13-118-Fc or TACI 13-118-Fc 81. These findings are consistent with the mechanism of action and relative in vitro potency of the four TACI-Fc test articles. The results further support that the 26 TACI CRD2-Fc fusion proteins demonstrate favorable characteristics, including higher serum exposure and more potent immunosuppressive activities, even as compared to the WT TACI-Fc fusion proteins. These results may support lower clinical doses and/or longer dosing intervals than WT TACI-Fc therapeutics, including for the treatment of multiple autoimmune and inflammatory diseases, particularly B cell-related diseases such as systemic lupus erythematosus (SLE), Sjogren's syndrome (SjS), and other connective tissue diseases.

### Example 12. Clinical Dose Selection and Pharmacokinetic Modeling

This Example describes the selection of a clinical dose and pharmacokinetic modeling of the exemplary test article 26 TACI CRD2-Fc.

Human pharmacokinetic (PK) was predicted based on the PK data from the cynomolgus monkey study described in Example 11, and allometric scaling method. A linear PK at different dose levels was assumed to predict human exposure. The clinical dose levels were selected based on predicted human PK and in vitro inhibition constant (IC) values from BAFF blockade in the Jurkat/NF-kB/TACI assay described in Example 2.

### PK Modeling

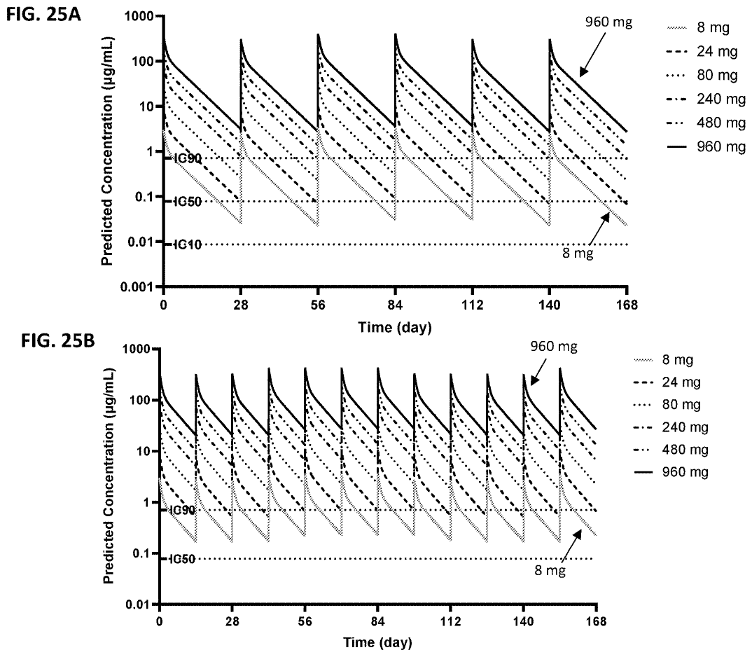

A two-compartment PK model was used to fit the observed PK data in the cynomolgus monkey study described in Example 11. The human PK parameters were predicted using the allometric scaling based on body weight and PK parameters estimated in monkeys. FIG. 25A-25B depict the predicted human PK profiles after repeated IV dosing every four weeks (FIG. 25A) or every two weeks (FIG. 25B). The lowest clinical dose (8 mg) was determined based on the predicted trough serum concentration, and resulted in greater than the IC₁₀ value (0.0087 μg/mL) and less than IC₅₀ value (0.078 μg/mL) following every four weeks of intravenous dosing (FIG. 25A), or greater than the IC₅₀value (0.078 μg/mL) and less than IC₉₀ value (0.705 μg/mL) following every two weeks of intravenous dosing (FIG. 25B).

### Example 13. Assessment of TACI Inhibition of Class-Switched Memory B Cell, Plasma Cells, and Ig Secretion

This Example describes studies assessing primary human B cell differentiation and immunoglobulin (Ig) secretion in vitro. Assessments of activity included B cell maturation as assessed by flow immunophenotyping, and measurement of secreted Ig including IgA, IgM, IgG2, in the culture supernatants.

Total CD19+ B cells were isolated from PBMCs (N=7 donors) using negative selection kits from StemCell Technologies. Isolated B cells were resuspended to approximately 2×10⁶ cells/mL in X-VIVO 15™ medium supplemented with 1×GlutaMAX, 1×P/S and rhIL-21 (50 ng/mL). CD40L was added to B cells at a concentration of 2 nM, and cells were plated into 12-well plates (2 mL/well). 8×10⁶-4.8×10⁷ total B cells were plated for each donor depending on the number of B cells isolated. Cells were incubated 3 days at 37° C. with 5% CO₂. On Day 3, B cells were harvested from the 12-well plates. Wells were washed with 1 mL/well PBS and treated 10 min with 37° C. Versene (1 mL/well) to remove adherent cells. Washes and detached cells were pooled with the other harvested cells for each respective donor. Cells were centrifuged and the media removed. Cell pellets were washed with 5-25 mL volumes DPBS. Cells were suspended in 1-5 mL DPBS and counted.

Activated B cell concentrations were adjusted to 1×10⁷/mL in DPBS. Equivalent volumes of CFSE (0.5 pM) in DPBS were added to cells (0.25 pM final). Cells were incubated 10 min at 37° C. After 10 min, 1-5 mL FBS were added, and cells were incubated for 5 min at 37° C. to quench labeling. Cells were washed twice with a 5-fold volume of X-VIVO 15™. After the second wash, cells were suspended in 1-2 mL X-VIVO 15™ and counted.

B cells (+/−CFSE) were suspended at 0.3-1.0×10⁶ cells/mL (0.3-1.0×10⁵/test) in 37° C. serum-free X-VIVO 15™ medium supplemented with 1×GlutaMAX, 1×P/S and rhIL-21 (20 ng/mL). 100 μL volumes of B cells were added to prepared microplates containing 5 APRIL and BAFF (10 nM) in the presence of titrated test articles Fc control, anti-APRIL mAb BION-1301 (e.g. SEQ ID NO: 50 and 52 from U.S. Pat. No. 10,377,830), belimumab, 26 TACI CRD2-Fc (TACI vTD SEQ ID NO:26; Fc fusion SEQ ID NO: 167) or WT TACI-Fc sequences corresponding to atacicept (containing a WT TACI 30-110 SEQ ID NO:132; SEQ ID NO:130) or telitacicept (containing WT TACI 13-118, SEQ ID NO:131).

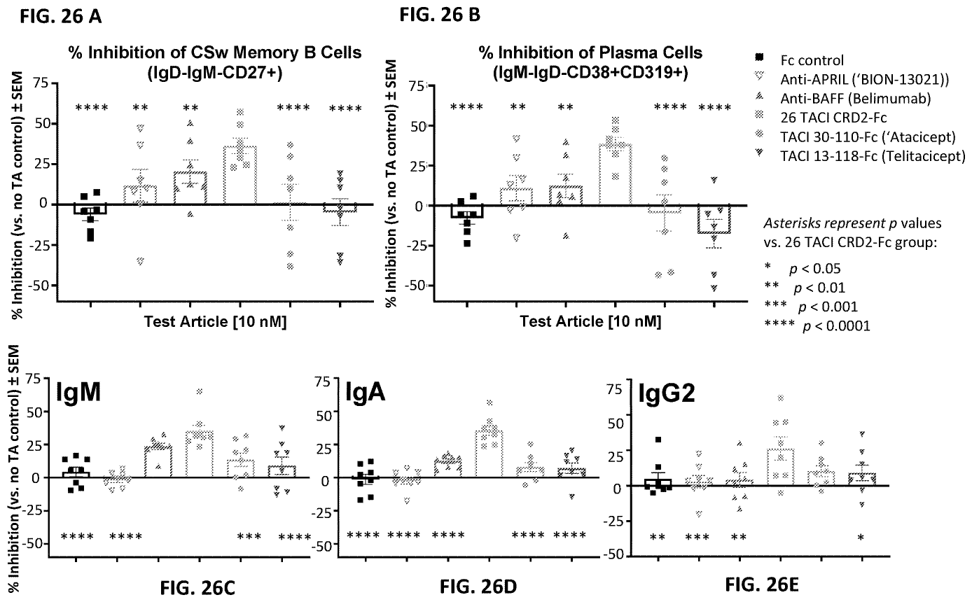

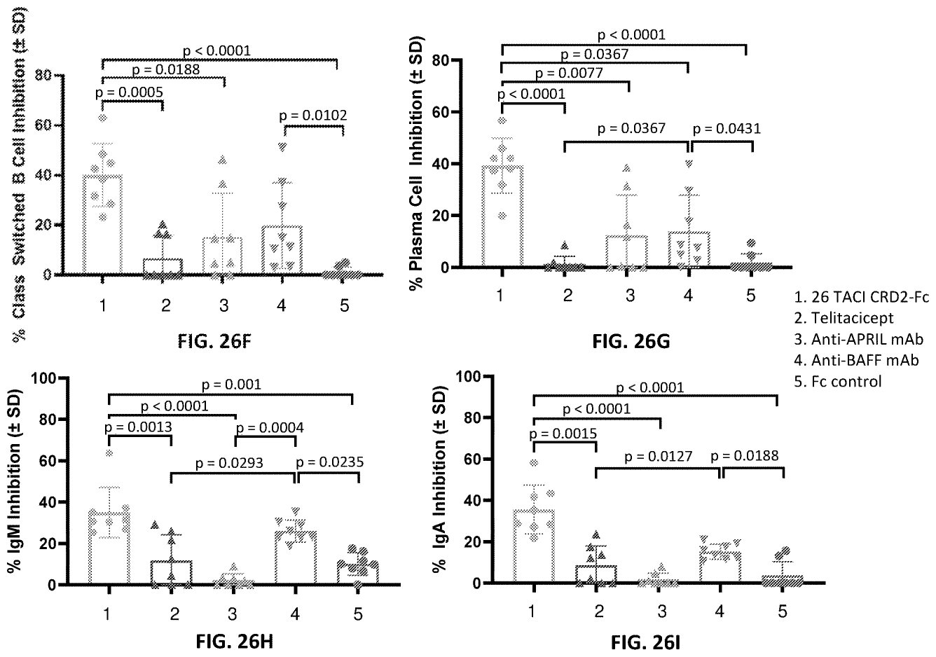

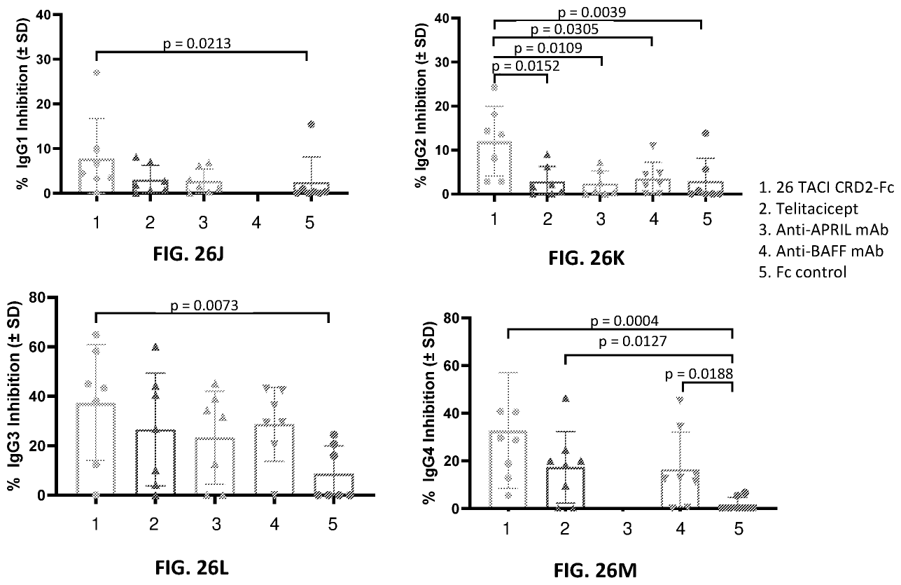

Cultured cells were analyzed by flow cytometry for CFSE and stained with antibodies for CD38, IgM, CD319, IgD and CD27. The percent (%) of class-switched memory B cells (IgD-IgM-CD27⁺), and plasma cells (IgM-IgD-CD38⁺ CD319⁺) were determined. The percent inhibition of class-switched memory B cells (FIG. 26A) or plasma cells (FIG. 26B) was determined by comparison of the % of such cells in the presence of test article versus in the absence of the test article. Data from 7 donors are shown, representing an average (±SEM) of 3 replicates for each condition. As shown, 26 TACI CRD2-Fc inhibits class-switched memory B cell and plasma cells more potently than WT TACI-Fc in primary human B cells.

To assess Ig secretion, supernatants from the cultures were collected and Ig secretion was quantitated by multiplex analysis. Media from the APRIL and/or BAFF-conditioned cultures were assayed using an immunoglobulin MILLIPLEX® kit (EMD Millipore, #HGAMMAG-301K) with magnetic beads and antibodies specific for detecting soluble IgM, IgG1, IgG2, IgG3, IgG4, and IgA. B cell culture supernatants (CM) were collected following centrifugation and diluted either 1:10 or 1:20 into MILLIPLEX® kit assay buffer. MILLIPLEX® kit immunoglobulin standard was solubilized into 500 μL water and serially diluted 1:3 into kit assay buffer. MILLIPLEX® map immunoglobulin positive control was solubilized in 250 μL water. Fifty L of standards, positive control, and diluted CM were plated onto 96-well Bio-Plex Pro™ Flat Bottom Plates. 50 μL of assay buffer alone was also added for the assay Blank control. All magnetic beads from the MILLIPLEX kit were sonicated and vortexed. A cocktail of the 6 Immunoglobulin-specific magnetic beads were prepared in kit assay buffer. The prepared bead cocktail (25 μL) was added to all wells. Plates were sealed and shaken vigorously at 500 rpm for 1 hr at 25° C. while protected from light. After a 1 hr incubation, plates with beads were washed using the magnetic bead washing protocol on a Cytek plate washer with prepared 1×MILLIPLEX® kit wash buffer. Kit antibody cocktail (25 μL/well) for detecting soluble IgA, IgM, IgG1, IgG2, IgG3, and IgG4 was first captured by the magnetic beads and then added to the plates. Plates were sealed and shaken vigorously at 500 rpm for 30 min at 25° C. while protected from light. After 30 min, 25 μL/well 1×SA-PE was added and sealed plates returned to the shaker. Prior to reading the reacted magnetic beads and their fluorescence signal on the calibrated LUMINEX® instrument, plates were seated onto a plate magnet for 1 min. Detection reagents were flicked off and 150 μL/well MAGPIX® drive fluid was added. LUMINEX® was programmed to read 100 beads minimally of each analyte and measure PE fluorescence for the 6 individual beads. Standard curves were generated using GraphPad Prism from Standard ng/mL concentrations and mean fluorescence intensities (MFIs) for each analyte. Secreted Ig levels in the CM were interpolated from the standard curves in GraphPad Prism and back-calculated for their respective dilutions. These results were further analyzed in GraphPad Prism for IC₅₀ calculation using a 4-parameter curve fit.

For APRIL+BAFF cultures, the percent inhibition of Ig secretion was determined using the following formula: ([Median APRIL+BAFF Ig value −Experimental Ig value]/Median APRIL+BAFF Ig value)×100. Percent inhibition was calculated relative to APRIL-only and BAFF-only wells for IgM (FIG. 26C), IgA (FIG. 26D), and IgG2 (FIG. 26E). Data represent an average (±SEM) of three replicates for each condition (*p<0.05, **p<0.01, ***p<0.001, ****p<0.0001). The results demonstrated that 26 TACI CRD2-Fc inhibits Ig secretion more potently than WT TACI-Fc in primary human B cells. Similar inhibition by 26 TACI CRD2-Fc was observed for other IgG subtypes (IgG1, IgG3, and IgG4). Percent inhibition of IgG1 and IgG4 could not be calculated for belimumab or BION-1301, respectively, as the multiplex kit detects the Fc contained within the test article.

In another experiment, the ability of 26 TACI CRD2-Fc to affect primary human B cell proliferation, differentiation, and Ig secretion in vitro was tested. Purified human pan B cells were first stimulated for 3 days with recombinant CD40L and then plated for 4 days in the presence of exogenous BAFF, APRIL, and test articles. As compared to telitacicept, anti-APRIL mAb, or anti-BAFF mAb, 26 TACI CRD2-Fc more potently inhibited expansion of total B cells and various key B cell subsets, including class switched memory B cells and plasma cells (FIGS. 26F and 26G). This potent inhibition of B cell expansion and survival by 26 TACI CRD2-Fc also correlated with significant reductions in Igs secreted into the culture media (FIGS. 26H-26K). These findings are consistent with the ability of 26 TACI CRD2-Fc to inhibit the differentiation of naïve B cells to plasma cells with activity superior to anti-BAFF or anti-APRIL mAbs, confirming that co-inhibition of both APRIL and BAFF results in greater suppression of Ig secretion compared to blockade of either cytokine alone. In addition, 26 TACI CRD2-Fc demonstrated superior activity as compared to telitacicept in this assay, consistent with its improved affinity for APRIL and BAFF

### Example 14. Assessment of TACI Inhibition on Plasma Cell Numbers in Mice and Non-Human Primates

The effect of the exemplary TACI-Fc designated 26 TACI CRD2-Fc on plasma cell numbers was assessed in mouse and non-human primate models.

For assessment in mice, collagen-induced arthritis (CIA) was induced in male DBA/1 mice by immunization with bovine collagen/CFA on Day 0 and bovine collagen/CFA booster on Day 18. CIA is mediated by both T cells and antibodies (B cells).

Mice were dosed with TACI domain-containing molecules 26 TACI CRD2-Fc (TACI vTD SEQ ID NO:26; Fc fusion SEQ ID NO: 167) and TACI 13-118-Fc (corresponding to the TACI ECD portion in telitacicept set forth in SEQ ID NO:131 with effectorless IgG1 Fc, SEQ ID NO:241; see also SEQ ID NO:3 of U.S. Pat. No. 8,193,316). For comparison, mice also were dosed with mBAFF-R-Fc (UniProt Q9D8D0) and anti-mAPRIL monoclonal antibody (WO 2017/091683 A1 SEQ ID NO: 161 and 162. Mice received 6 doses of each test article twice weekly (10 mg/kg). Mice were sacrificed, and their spleens, bone marrow and lymph nodes were isolated for flow cytometry analysis of plasma cells.

Each sample was then stained for flow cytometry analysis of immune cell subsets using the following method: 1×10⁶ live cells were placed into a well of a 96-well plate (Corning, Cat. 3797; for a B cell-specific panel), centrifuged at 1500×g for 10 seconds, the supernatant removed, and the cell pellet washed twice with DPBS. The pellets were resuspended in 100 L of live-dead stain (LIVE/DEAD Fixable Aqua Dead Cell Stain Kit, Life Technologies Corp., 1:1000 dilution in DPBS) and incubated for 10 min in the dark at room temperature. Following two washes with flow cytometry buffer (175 L each), pellets were resuspended in Mouse BD Fc Block (diluted 1:50 with flow buffer), and incubated in the dark for an additional 5 min at RT. Without any additional washes, 50 L of a cocktail of the following flow cytometry antibodies (diluted in flow cytometry buffer) were added to each well of cells for the B panels. For the B cell panel, the following antibodies were combined for the cocktail: anti-mouse CD19 BUV395 (clone 1D3, Becton-Dickinson; 1:100), anti-mouse CD138 BV421 (clone 281-2, BioLegend Inc.; 1:100, final concentration), anti-mouse CD3E BV510 (clone 17A2, BioLegend Inc.; 1:100, final concentration), anti-mouse IgD BV605 (clone 11-26c.2a, BioLegend Inc.; 1:100, final concentration), anti-mouse B220 BV785 (clone RA3-6B2, BioLegend Inc.; 1:100, final concentration), anti-mouse CD95 FITC (clone SA367H8, BioLegend Inc.; 1:100, final concentration), anti-mouse CD23 PerCP Cy5.5 (clone B3B4, BioLegend Inc.; 1:100, final concentration), anti-mouse GL7 PE (clone GL7, BioLegend Inc.; 1:100, final concentration), anti-mouse Gr1 PE Cy7 (clone RB6-8C5, BioLegend Inc.; 1:100, final concentration), anti-mouse CD21 APC (clone 7E9, BioLegend Inc.; 1:100, final concentration), and anti-mouse IgM APC Cy7 (clone RMM-1, BioLegend Inc.; 1:100, final concentration). The cells were incubated with one of the antibody cocktails in the dark, on ice, with gentle mixing for 45 min, followed by two washes with flow cytometry buffer (175 L per wash). Cell pellets were resuspended in 200 L flow cytometry buffer and collected on an LSRII flow cytometer. Data were analyzed using FlowJo software version 10.2 (FlowJo LLC, USA) and graphed using GraphPad Prism software (Version 8.1.2). Key cellular subset identification analysis included: plasma cells (CD138^{high} TACI^{high})

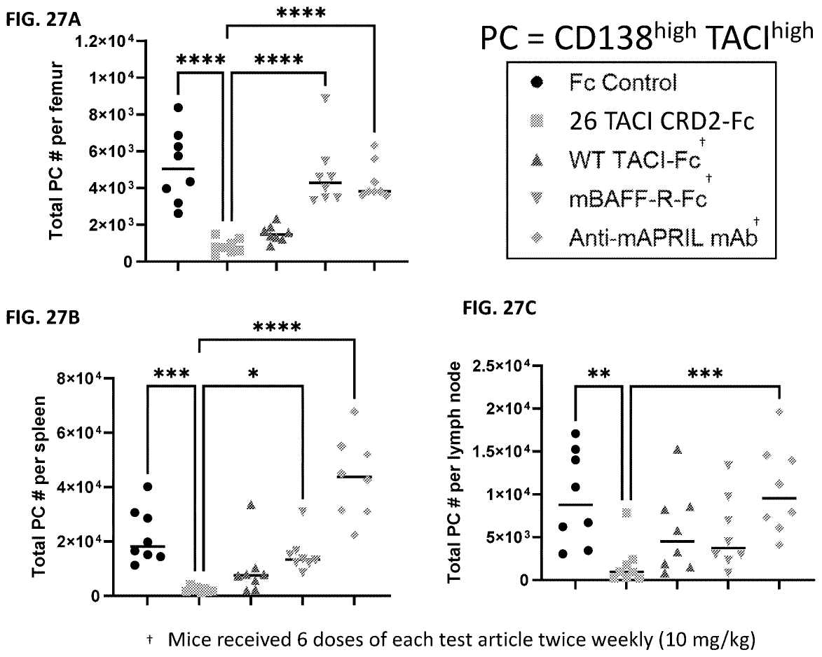

As shown in FIGS. 27A-C TACI CRD2-Fc significantly decreased the total plasma cell numbers in the bone marrow (FIG. 27A), spleen (FIG. 27B), and the lymph node (FIG. 27C), relative to the Fc control, WT TACI-Fc, mBAFF-R-Fc, and/or anti-mAPRIL monoclonal antibody.

For a non-human primate GLP 1-month toxicology study, as described in Example 17, 26 TACI CRD2-Fc (TACI vTD SEQ ID NO:26; Fc fusion SEQ ID NO: 167) was administered to cynomolgus monkeys via subcutaneous injections at dose levels 25 mg/kg, 75 mg/kg or 150 mg/kg once weekly of five consecutive weeks; control animals were injected with vehicle (buffer), alternating between IV and SC routes of administration. Bone marrow smears were examined at low magnification (200× and 400×) to review the cellularity of the smears and to locate an appropriate monolayer area in which to perform cell counting. Plasma cells and other nucleated cells were counted (using two keys) with the Unico® counter at 500× oil immersion to determine the number of plasma cells per 500 total nucleated cells; the percentage of plasma cells was calculated to the nearest decimal point by dividing the number of plasma cells by 500.

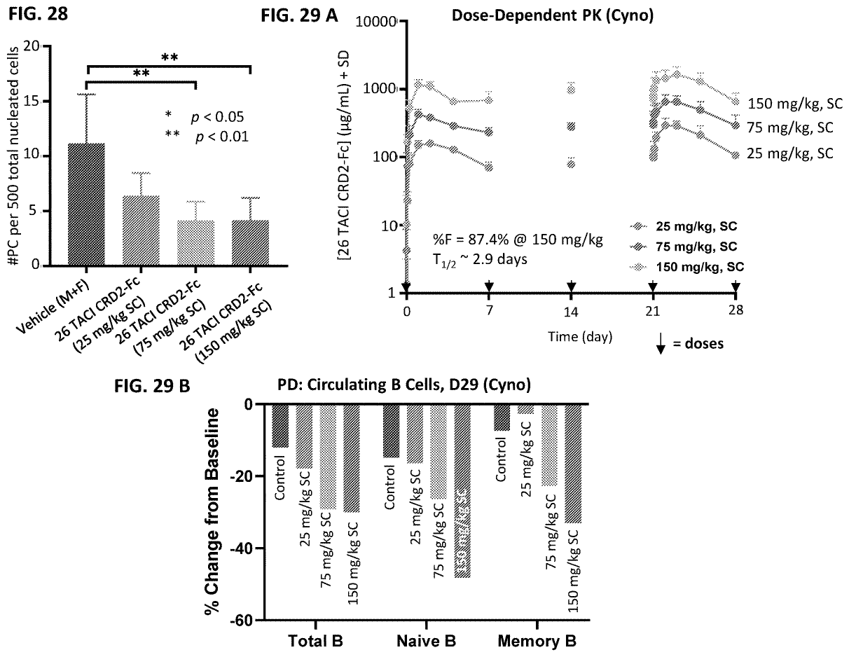

As shown in FIG. 28, the plasma cells were observed in low numbers, as expected, with some variation among the animals. Lower plasma cell counts were observed in animals dosed with 26 TACI CRD2-Fc relative to those from the vehicle control group, with a statistically significant decrease observed with ≥75 mg/kg by SC. These lower plasma cell counts are consistent with an effect from 26 TACI CRD2-Fc administration.

### Example 15. Multiple Dose Toxicology Study of TACI vTC-Fc in Sprague Dawley Rats

This Example describes a 1-month GLP toxicology study in Sprague Dawley (SD) following of the exemplary TACI vTD-Fc designated 26 TACI CRD2-Fc (SEQ ID NO:167), when administered by 5 weekly doses of subcutaneous injection or intravenous slow bolus injection for 4 weeks to SD rats.

Male and female SD rats were divided into five groups (Groups 1 to 5). Groups 2 to 5 (terminal population) included ten males and ten females per group and Groups 4 and 5 (recovery population) included five males and five females per group. Animals were dosed by subcutaneous injection into the interscapular area once weekly on Days 1, 8, 5, 22, and 29 (Groups 1 to 4) and by slow intravenous (IV) injection over 1 min via tail vein once weekly for five weeks, i.e. on Days 1, 8, 15, 22, and 29 (Groups 1 and 5). For control animals, the slow IV injection will be performed after the subcutaneous injection. Dose formulations were administered using a temporary catheter and syringe. The vehicle control article, 10 mM acetate, 3% proline, 0.015% polysorbate 80, pH 5.2, was administered to 10 males and 10 females Sprague Dawley rats (Group 1; terminal population) and 5 males and 5 females (Group 1; recovery population). A separate population was assigned to the study for toxicokinetic assessments, which included 3 male and 3 female Sprague Dawley rats in Group 1, and 9 male and 9 female Sprague Dawley rats in Groups 2 to 5. Animals in Groups 2 to 4 were dosed via subcutaneous (SC) injection and animals in Group 5 were dosed via slow intravenous (IV) injection once weekly on Days 1, 8, 15, 22, and 29. Group 1 animals received the control article via SC injection followed by slow IV injection. Groups 2 through 5 received 26 TACI CRD2-Fc at dose levels of 25, 75, 200 mg/kg SC, and 200 mg/kg IV, respectively. Dose formulations were accurately prepared, based on the analytical results from preparations used for dosing on Days 1 and 29. Necropsy of the terminal population was performed on Day 30 and recovery necropsy will be performed on Day 127.

Safety endpoints included clinical observations, detailed examinations, food consumption evaluation, body weights, ophthalmology, hematology, coagulation, serum chemistry, serum immunoglobulins, urinalysis, and anti-drug antibodies (ADA). Blood was collected at multiple time points to characterize 26 TACI CRD2-Fc serum concentrations over time. At termination, gross observations and organ weights were recorded, and tissues were collected for microscopic evaluation.

Toxicokinetic parameters were imported into Phoenix WinNonlin software (Pharsight Corp/Certara) for 26 TACI CRD2-Fc (concentration and time). Non-compartmental analysis was applied on the mean composite serum concentrations using nominal collection times and nominal dose times.

26 TACI CRD2-Fc-related serum chemistry changes in male and female rats administered ≥25 mg/kg included minimally lower globulin concentrations and minimally higher albumin to globulin (A/G) ratios on Day 30. Immunoglobulin IgA and IgM concentrations were lower than acclimation values on Day 8 and were moderately to markedly below control values on Day 15 and Day 29, which contributed to overall lower globulin concentrations. Subcutaneous administration of 25 mg/kg to 200 mg/kg 26 TACI CRD2-Fc resulted in transiently lower IgG concentrations on Day 8; intravenous administration of 200 mg/kg 26 TACI CRD2-Fc resulted in persistently lower IgG concentrations through Day 29, in male and female animals. Significant decreases in mean spleen weights were noted in all treatment groups. 26 TACI CRD2-Fc-related microscopic findings were observed on Day 30 in the spleen, lymph nodes, and injection site. Decreased lymphocyte cellularity in the spleen and lymph nodes was noted in animals administered 200 mg/kg via SC or IV injection. The decreased cellularity in the spleen correlated with decreased spleen weights. Increased incidence and severity of decreased lymphocyte cellularity of the follicles in the lymph nodes (mesenteric and mandibular) was also observed in animals treated with 200 mg/kg 26 TACI CRD2-Fc by SC or IV injection. Subcutaneous inflammatory changes (mononuclear cell infiltrates and/or fibroplasia) were slightly increased at the SC injection site in animals that received 200 mg/kg compared to those at the control article injection site, which was considered to be an exacerbation of commonly observed procedure-related changes.

In conclusion, 26 TACI CRD2-Fc administered by subcutaneous injection or intravenous injection to Sprague Dawley rats at 25, 75, 200 mg/kg SC, and 200 mg/kg IV for 4 weeks resulted in lower serum globulin attributed to decreased immunoglobulin (IgA, IgM and IgG) concentrations and decreased lymphocyte cellularity in the spleen and lymph nodes, all consistent with the mechanism of action of 26 TACI CRD2-Fc. As none of the effects were considered adverse, the NOAEL (Non-Observable-Adverse-Effect-Level) was determined to be 200 mg/kg by subcutaneous or intravenous injection.

### Example 16. Multiple Dose 1-Month GLP Toxicology Study of TACI vTD-Fc in Cynomolgus Monkeys

This Example describes a 1-month GLP toxicology study in cynomolgus monkey to examine the effects of the exemplary TACI vTD-Fc designated 26 TACI CRD2-Fc (SEQ ID NO:167), when administered by once weekly subcutaneous injection for 4 weeks (5 total doses) to cynomolgus monkeys.

Male and female cynomolgus monkeys were divided into groups. Animals were dosed by subcutaneous injection (Groups 1 to 4) once weekly for five consecutive weeks on Days 1, 8, 15, 22 and 29. Group 1 animals received vehicle control (10 mM acetate, 3% proline, 0.015% polysorbate 80, pH 5.2). Group 2 to 4 animals were administered 26 TACI CRD2-Fc (SEQ ID NO:167), at dose levels of 25, 75, or 150 mg/kg, respectively. Animals in an additional group 5 were administered 26 TACI CRD2-Fc (SEQ ID NO:167), at dose level 150 mg/kg via intravenous infusion.

Toxicokinetic parameters were imported into Phoenix WinNonlin software (Pharsight Corp/Certara) for analysis for 26 TACI CRD2-Fc (concentration and time). Non-compartmental analysis was applied on the individual subject serum concentration using nominal collection times and nominal dose levels. Dose-dependent PK observed in this model is set forth in FIG. 29A. Additional parameters calculated were bioavailability 87.4% (% F) at 150 mg/kg and elimination half-life (T_{1/2}) of approximately 2.9 days.

Flow cytometry analysis was performed on peripheral blood samples collected on Days −8, 8, 15, 22, and 29 on control animals (Group 1) and animals treated with 25 mg/kg SC (Group 2), 75 mg/kg SC (Group 3), 150 mg/kg SC (Group 4), and 150 mg/kg IV (Group 5) of 26 TACI CRD2-Fc (SEQ ID NO:167) collected on Day −8 before dosing (baseline) and on Days 8, 15, 22, and 29 after dosing. Cell immunophenotyping was performed on collected peripheral blood and relative percentages and absolute counts for populations for CD3-CD20+ (total B cells), CD3−CD20+CD21+CD27− (naïve B cells), and CD3−CD20+CD21+CD27+ (memory B cells). To determine TACI-related changes, averages of relative percentages and absolute counts values per group after dosing were compared to the baseline value (Day −8) of respective treatment groups and to trends observed in control Group 1. Flow cytometry analysis indicated multiple changes in the absolute counts and relative percentage values of CD3−CD20+ B cells and subsets following 26 TACI CRD2-Fc administration (FIG. 29B). All of the observed changes were characterized by a decrease in relative percentages or absolute counts. Since these decreases were observed in multiple B cell subsets and in both males and females, to the results are consistent with an effect of the 26 TACI CRD2-Fc administration. For populations CD3−CD20+CD21+ and CD3−CD20+CD21+CD27−, moderate (two-fold) decreases were observed in absolute counts compared to baseline and Group 1 values starting on Day 15 in both male and female treatment groups.

Serum cytokines were also measured as a non-GLP exploratory endpoint in this study. Frozen serum samples collected predose (Day 1), then 2 hours (Day 1), 6 hours (Day 1), and 24 hours (Day 2) following the first dose of 26 TACI CRD2-Fc were provided frozen on dry ice. Serum samples (from 42 animals, N=168 samples total) were thawed, vortexed for 30 seconds, then stored at 4° C. prior to assay. Samples were plated in duplicate wells (25 μL/well) and concentrations of a panel of cytokines were measured using the Millipore Milliplex NHP Cytokine Assay kit (catalog #PRCYTA-40K; Lot #3739326) and analyzed with a Luminex 200® System with xPONENT® 4.2 software (EMD Millipore, Burlington, MA). As compared to samples from vehicle treated control animals, and to intra-animal pre-dose measurements, no significant changes were induced by 26 TACI CRD2-Fc treatment in any of the cytokines evaluated (IL-2, IL-4, IL-6, IL-8, IL-10, IFNγ, or TNFα).

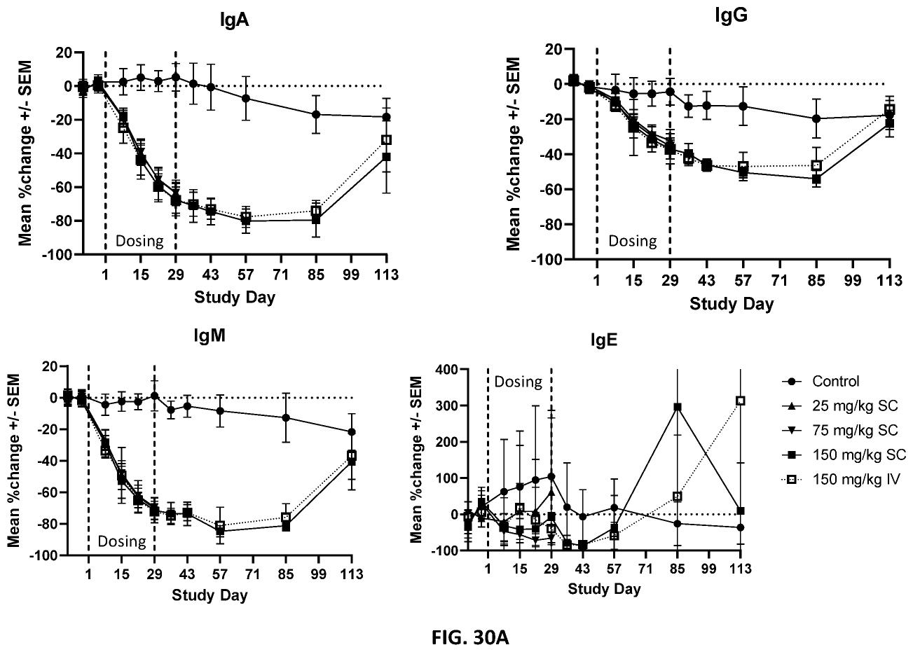

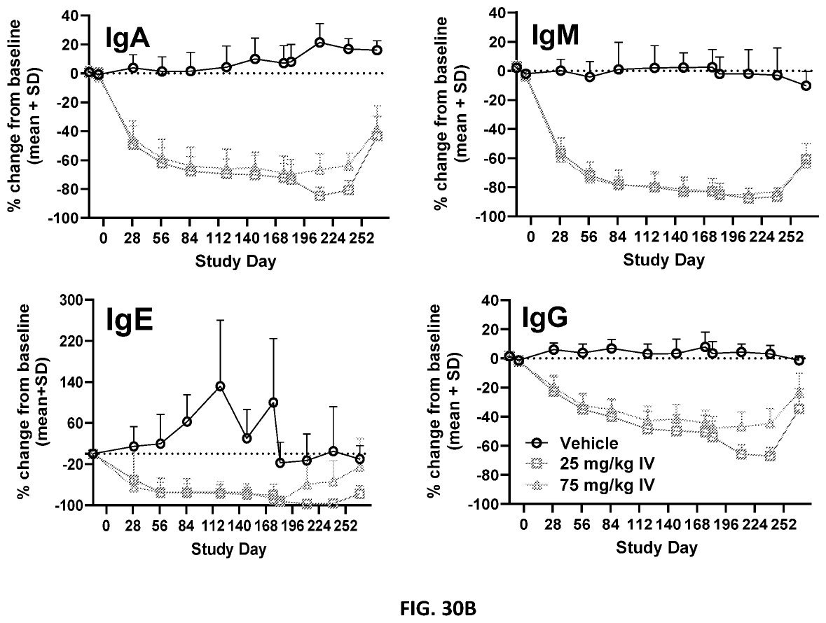

In conclusion, 26 TACI CRD2-Fc administered by subcutaneous injection or intravenous injection to cynomolgus monkeys at 25, 75, and 150 mg/kg SC or 150 mg/kg IV for 4 weeks resulted in non-adverse lower mean total protein and globulin values. Decreased serum globulin (secondary to decreased IgA, IgM and IgG) concentrations, possibly related to lower B cell populations and plasma cell counts in the marrow, and consistent with the mechanism of action of 26 TACI CRD2-Fc were observed at all dose levels in a 1-month study (FIG. 30A) and a 6-month study (FIG. 30B). As such, the NOAEL (Non-Observable-Adverse-Effect-Level) was considered to be 150 mg/kg by subcutaneous injection.

### Example 17. Evaluation of TACI vTD-Fc in a Chronic Graft Versus Host Disease (cGVHD) Model of Lupus

This Example describes the evaluation of the in vivo activity of TACI vTD-Fc designated 26 TACI CRD2-Fc (SEQ ID NO:167) in comparison to a WT TACI (13-118) Fc containing a wild-type Fc that can mediate effector function (SEQ ID NO: 240), when administered using a repeat dosing regimen in the bm12-to-C57BL/6NJ mouse inducible model of SLE. In this model, splenocyte suspensions from female I-A^{bm12}B6(C)-H2-Ab1^{bm12}/KhEgJ (‘bm12’) mice were adoptively transferred via intraperitoneal delivery into female C57BL/6NJ recipient mice. H2-Ab1^{bm12} differs from H2-Ab1^{b} by 3 nucleotides, resulting in an alteration of 3 amino acids in the β-chain of the MHC class II I-A molecule. Alloactivation of donor bm12 CD4+ T cells by recipient antigen presenting cells leads to chronic GVHD with symptoms closely resembling SLE, including autoantibody production, changes in immune cell subsets, and mild kidney disease. Increased serum IgG and anti-dsDNA occur approximately 1-2 weeks post-transfer of splenocytes. Glomerulonephritis can develop late in the model (12-14 weeks post-transfer) whereas glomerular immune complex deposition, largely composed of autoantigens bound to IgG1, IgG2b, IgG2c, and IgG3 antibodies, can be detected as early as 4 weeks post-transfer. Endpoints of this study included immune cell subset composition in the spleen and renal IgG immune complex deposition in the kidney.

To begin the study, spleens from 40 bm12 mice, and inguinal lymph nodes from 20 of those mice, were processed aseptically to single cell suspensions in RPMI media, pooled, and injected via intraperitoneal (IP) delivery to 39 C57BL/6 ‘recipient’ mice (Groups 1-4) as shown in Table E37. A total of 8 mL of pooled lymph node cells/splenocytes were prepared and each of the 39 recipient mice received 0.2 mL of the pooled bm12 cells. C57BL/6 ‘recipient’ mice received 1 of 3 test articles (Groups 1-4) by IP injection, with the first dose being 5 days after the transfer of bm12 splenocytes; the last dose was administered 6 days prior to termination, i.e. doses 2×/week Day 5 to Day 88 (last dose during week 14). Six C57BL/6 and 5 bm12 mice were retained for use as naïve, untreated controls over the course of the study.

TABLE E37
Test Article Description and Dose Regimen

| | | | Dose | Dose Schedule | |
|---|---|---|---|---|---|
| | # of | | Level | (D = Study | Route of |
| Group # | mice | Test Article(s) | (μg) | Day) | Delivery |
| 1 | 13 | Fc control | 80* | 2x weekly, | IP |
| | | | | D 5 start | |
| 2 | 13 | 26 TACI | 200 | 2x weekly, | IP |
| | | CRD2-Fc | | D 5 start | |
| 3 | 13 | WT TACI | 240 | 2x weekly, | IP |
| | | (13-118) Fc | | D 5 start | |
| 4 | 6 | None | N/A | N/A | N/A |
| | | (naïve BL/6) | | | |

*molar matched to Fc control
N/A = not applicable

Blood was collected every 1-2 weeks and processed to serum and test article concentrations were measured to confirm expected exposure. Mice were sacrificed at week 14 and blood was terminally collected under isoflurane anesthesia. Spleens were collected at termination from each mouse, weighed and processed to single-cell suspensions for immunophenotyping by flow cytometry. Total cell counts were obtained on a Cellometer (Nexcelom Bioscience).

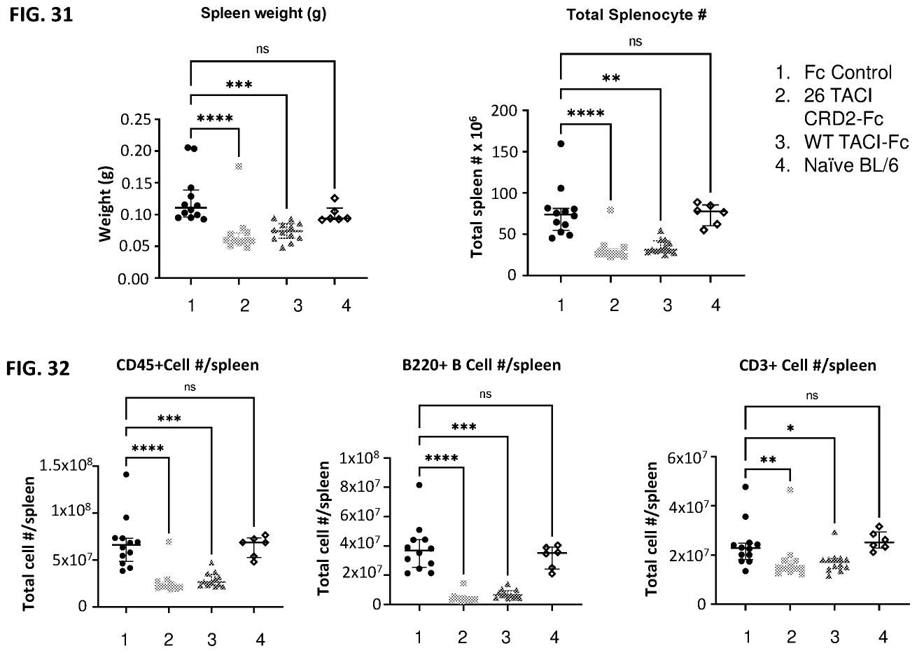

As shown in FIG. 31, administration of WT TACI-Fc and 26 TACI CRD2-Fc significantly reduced spleen weights as well as total number of spleen cells as compared to the controls treated mice. Immunophenotyping of splenocytes by flow cytometry revealed highly significant reductions in the numbers of CD45⁺ and B220⁺ in the WT TACI-Fc and 26 TACI CRD2-Fc treated groups (FIG. 32). A modest reduction in the number of CD3⁺ T cells in was also observed in both treatment groups.

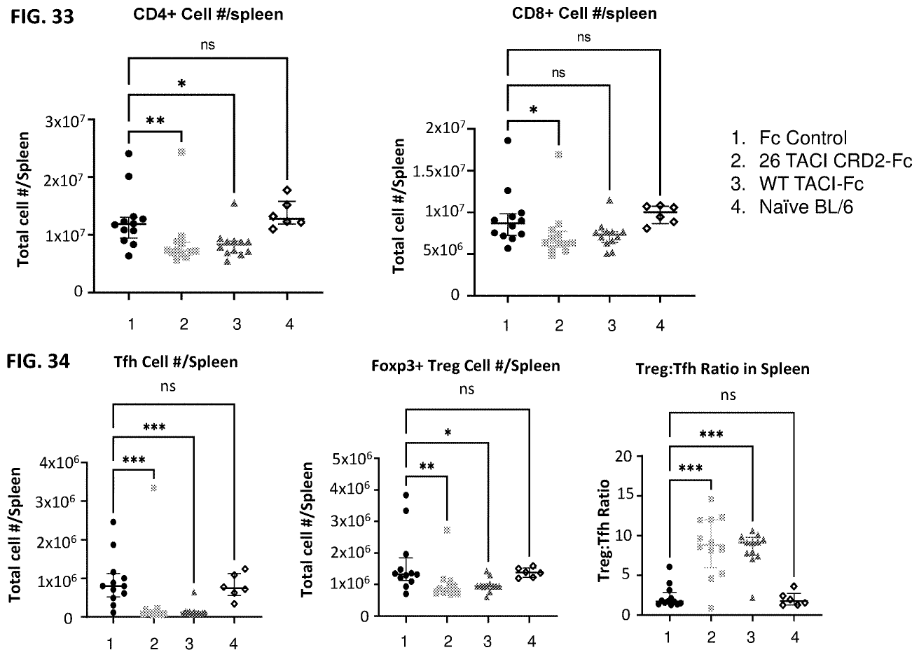

As shown in FIG. 33, WT TACI-Fc and 26 TACI CRD2-Fc significantly decreased the numbers and percentages of CD4⁺ and CD8⁺ T cells in the spleen. Further, the two test articles reduced the CD4⁺ T cell subsets that are important in antibody-mediated disease (FIG. 34). Mild reductions in the number of regulatory T (Treg) cells were observed; however, as both WT TACI-Fc and 26 TACI CRD2-Fc significantly decreased the number of follicular T helper (Tfh) cells, treatment with each test article also increased the ratio of Treg to Tfh cells (FIG. 34).

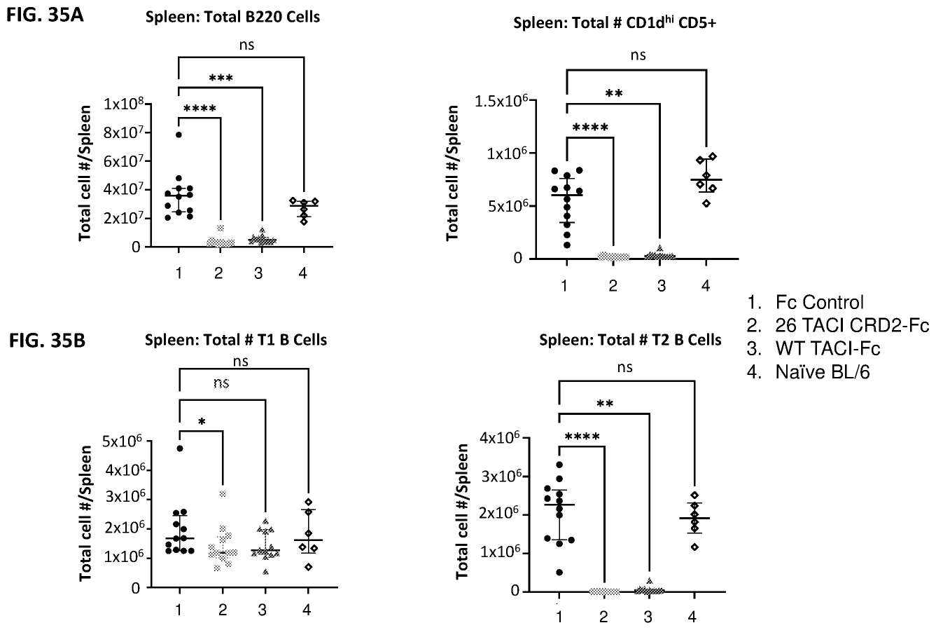

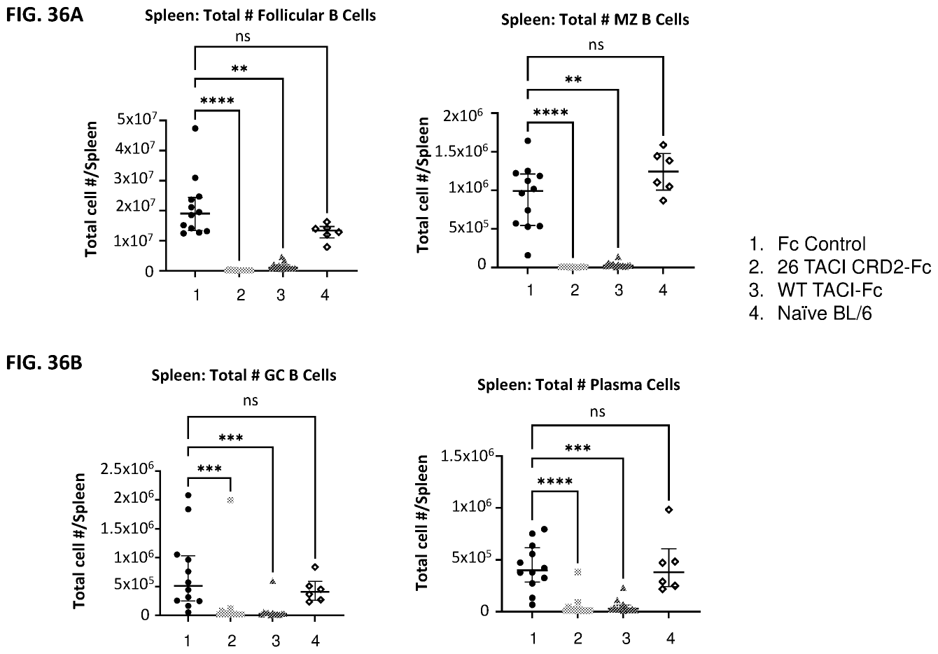

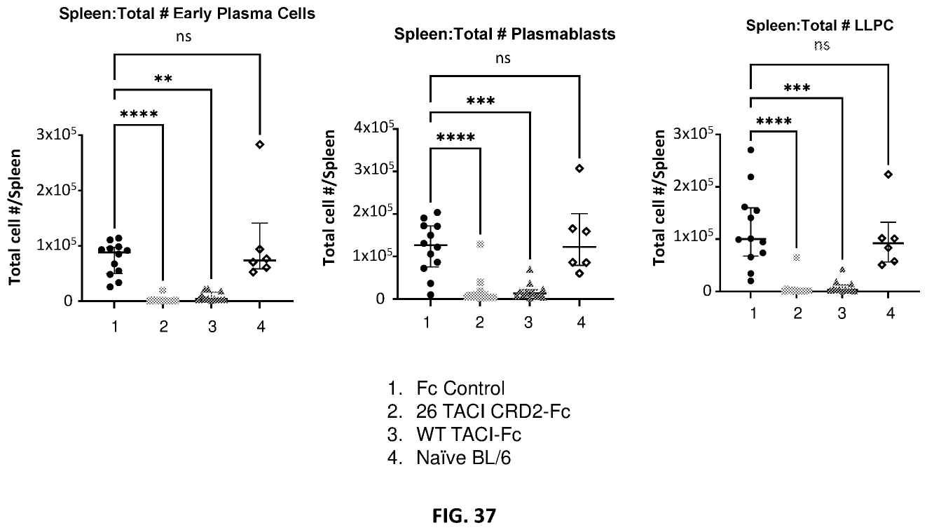

As shown in FIG. 35A, WT TACI-Fc and 26 TACI CRD2-Fc significantly decreased B cell numbers, including numbers of CD1d^{hi}CD5⁺ B-1 cells. 26 TACI CRD2-Fc had little effect on the number of Transitional-1 (T1) B cells, but dramatically reduced the number of Transitional-2 (T2) B cells, as expected based on the reliance of B cells beyond the T-1 stage of development on BAFF and APRIL for survival (FIG. 35B). Thus, WT TACI-Fc and 26 TACI CRD2-Fc also significantly decreased the numbers of follicular and marginal zone (MZ) B cells (FIG. 36A), germinal center (GC) B cells, and plasma cells (FIG. 36B). 26 TACI CRD2-Fc, and less so WT TACI-Fc, significantly decreased the number of various immunoglobulin-secreting B cells, including early plasma cells, plasmablasts, and long-lived plasma cells (LL-PC) (FIG. 37).

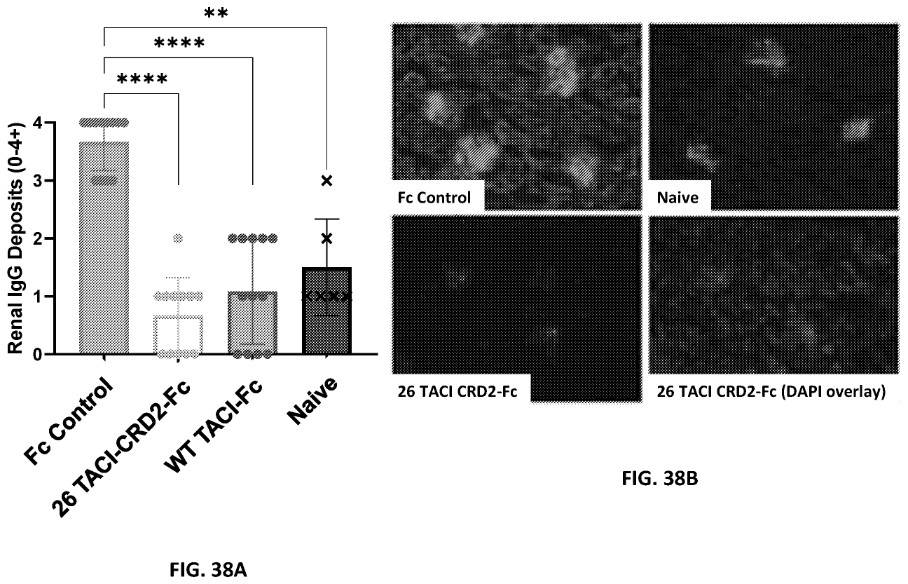

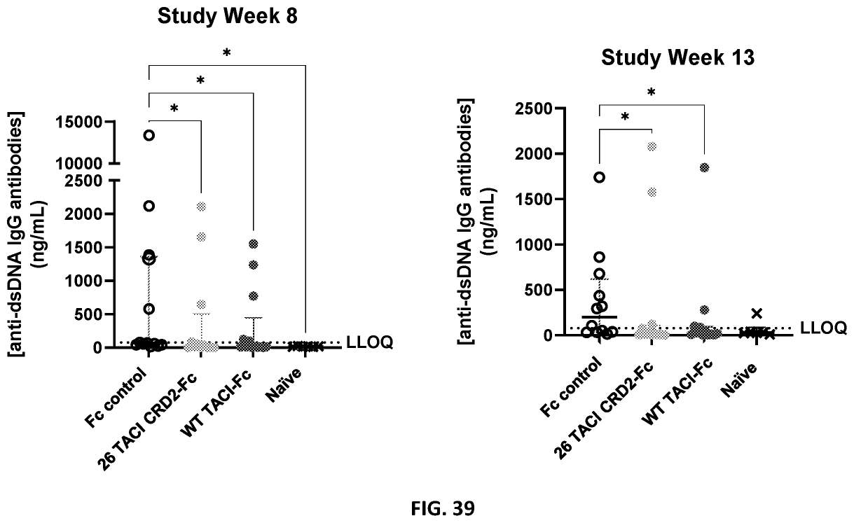

For FIG. 38, kidneys were collected at termination from each mouse and frozen in optimal cutting temperature compound (OCT) block before sectioning and immunohistochemical (IHC) staining with a fluorescently-labeled antibody specific for mouse IgG. Glomerular IgG deposits were significantly elevated in this model at the end of the study (Day 95), and 26 TACI CRD2-Fc treatment led to highly significantly decreased renal IgG immune complex deposits as compared to the Fc control (FIGS. 38A and 38B). Furthermore, WT TACI-Fc and 26 TACI CRD2-Fc significantly reduced the serum titers of anti-dsDNA autoantibodies as compared to the Fc control at weeks 8 and 13 (FIG. 39).

Results demonstrate that 26 TACI CRD2-Fc significantly reduced splenic Tfh, GC B cells, and PC populations that are key in the cGVHD model and in antibody-mediated disease. 26 TACI CRD2-Fc also significantly reduced anti-dsDNA antibodies in serum and inhibited IgG immune complex deposition in the kidneys.

### Example 18. Evaluation of TACI vTD-Fc in a H-2^{bm12} Mouse Model of Autoantibody-Related Glomerulonephritis

This Example describes the evaluation of the activity of TACI vTD-Fc designated 26 TACI CRD2-Fc (SEQ ID NO:167) compared to Fc control in the bm12 mouse model of chronic GVHD. Alloactivation of donor T cells in the GVHD model leads to clinical, serological and histopathological manifestations that mimic multiple systemic autoimmune diseases, including autoantibody-related glomerulonephritis.

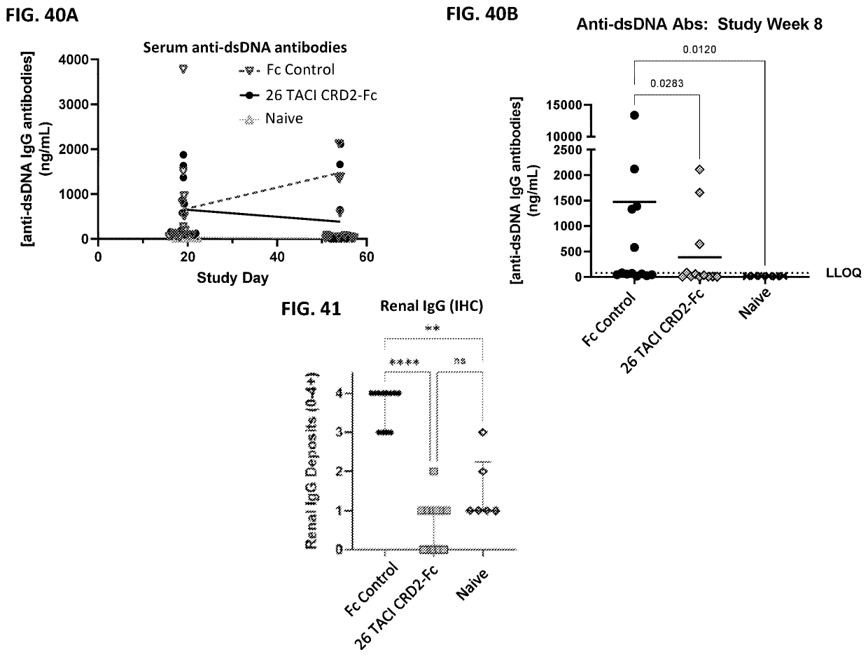

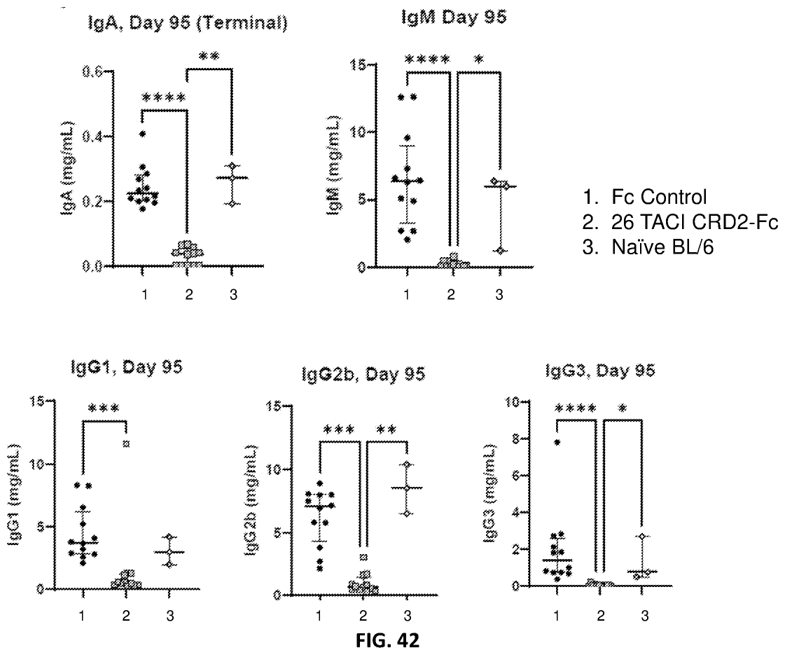

To begin the study, mice were dosed twice weekly for 12.5 weeks with TACI-Fc or Fc control. Naïve C57BL/6NJ mice were included as control animals. Endpoints evaluated included anti-double stranded (ds) DNA antibodies, analysis of splenic immune cell subsets, and renal IgG deposition via immunohistochemistry. Results demonstrated that 26 TACI CRD2-Fc treatment significantly reduced anti-dsDNA autoantibodies (FIG. 40, see also FIG. 40B), glomerular IgG immune deposits (FIG. 41) compared to Fc controls. Immunophenotyping of splenocytes demonstrated that 26 TACI CRD2-Fc treatment resulted in significant reductions of key immune cell subsets, including germinal center (GC), marginal zone (MZ), mature T2, and follicular B cells, antibody-producing plasma cells, and plasma cell subsets. Significant reductions were also observed in CD4+ follicular helper T cells, regulatory T cells, CD4+ effector memory T cells, and CD4+ and CD8+ central memory T cells. 26 TACI CRD2-Fc also significantly reduced serum levels of IgA, IgM, IgG1, IgG2b, and IgG3 (FIG. 42).

Results demonstrate that 26 TACI CRD2-Fc significantly suppressed the formation of autoantibodies and significantly reduced glomerular IgG deposition as compared to Fc control treatment; in addition to inhibiting the expansion of key B and T cell subsets.

### Example 19. Multiple Dose 26-Week Toxicology Study of TACI vTD-Fc in Sexually Mature Cynomolgus Monkeys

This Example describes a toxicology study in sexually mature cynomolgus monkeys to evaluate the potential toxicity of TACI vTD-Fc designated 26 TACI CRD2-Fc (SEQ ID NO:167) when administered by once weekly intravenous infusion for 26 weeks (26 doses) followed by a 12-week recovery.

The experimental study design is shown in Table E38.

TABLE E38
Experimental Study Design

| | | Dose | Ter- | Ter- | Re- | Re- |
|---|---|---|---|---|---|---|
| | Test | Level | minal | minal | covery | covery |
| Group | Material | (mg/kg) | (Male) | (Female) | (Male) | (Female) |
| 1 | Control | 0 | 4 | 4 | 2 | 2 |
| 2 | 26 TACI | 25 | 4 | 4 | 2 | 2 |
| | CRD2-Fc | | | | | |
| 3 | 26 TACI | 75 | 4 | 4 | 2 | 2 |
| | CRD2-Fc | | | | | |

TACI-Fc was well tolerated with all animals surviving to scheduled necropsy. No TACI-Fc-related changes in clinical signs, vital signs, body weight, menstrual cycles, testicular volume, semen, ophthalmology, coagulation, urinalysis, anatomic and gross pathology, organ weights, and histopathology were observed.

Consistent with the anticipated effects of 26 TACI CRD2-Fc, changes in immunoglobulins and bone marrow plasma cells were observed. Lower plasma cells were observed in the bone marrow smears from most of the animals in the 25 and 75 mg/kg dose groups at terminal necropsy. Related to the plasma cell observations, statistically significant changes in serum chemistry were observed in animals administered with 26 TACI CRD2-Fc, including, mild, moderate, and/or marked, generally progressive, dose-dependent decreases in IgG, IgM, and IgA values from Day 29 through Day 183. The immunoglobulin changes were associated with statistically significant, minimal to mild, decreases in mean values for globulin and total protein concentrations and minimal to mild increases in mean albumin/globulin (A/G) ratios in animals administered with 26 TACI CRD2-Fc from Day 29 through Day 183. No other TACI-Fc-related changes were observed in the hematology parameters assessed, except for minimally lower mean lymphocyte counts within the normal range of healthy monkeys, in 75 mg/kg males on Day 183, attributable to two animals.

Statistically significant changes in total B cells (CD3−CD20+) were noted by immunophenotyping and are likely attributed to 26 TACI CRD2-Fc administration. Otherwise, there was no evidence of the effect of TACI-Fc administration in the other cell populations evaluated. NOAEL was observed to be 75 mg/kg/day.

### Example 20. Dual Inhibition of BAFF and APRIL in Mouse SRBC Challenge

This Example describes the assessment of exemplary 26 TACI CRD2-Fc fusion protein in an in vivo Sheep Red Blood Cell (SRBC) challenge in mice, including assessment of anti-SRBC IgG1 and plasma cells in bone marrow. Mice were administered sheep red blood cells (SRBC) followed by administration of provided 26 TACI CRD2-Fc fusion protein or comparator controls (including WT TACI 13-118-Fc, anti-murine APRIL antibody, murine BAFF-R-Fc or the combination of anti-murine April and murine BAFF-R-Fc). Mice were sacrificed on Day 15, and spleens and bone marrow were processed for immunophenotyping by flow cytometry. B cell responses were measured by assessing the anti-SRBC antibody titers in serum and percent of plasma cells in bone marrow. Serum titers of anti-SRBC IgM and IgG were also measured.

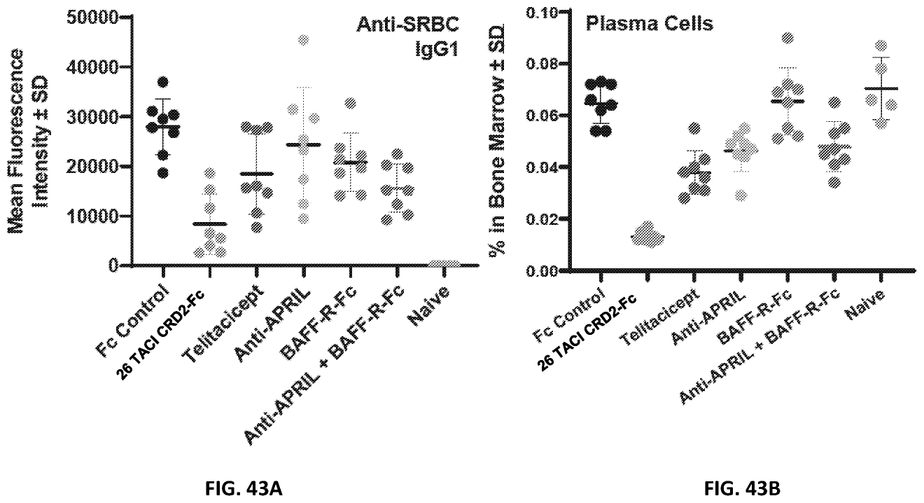

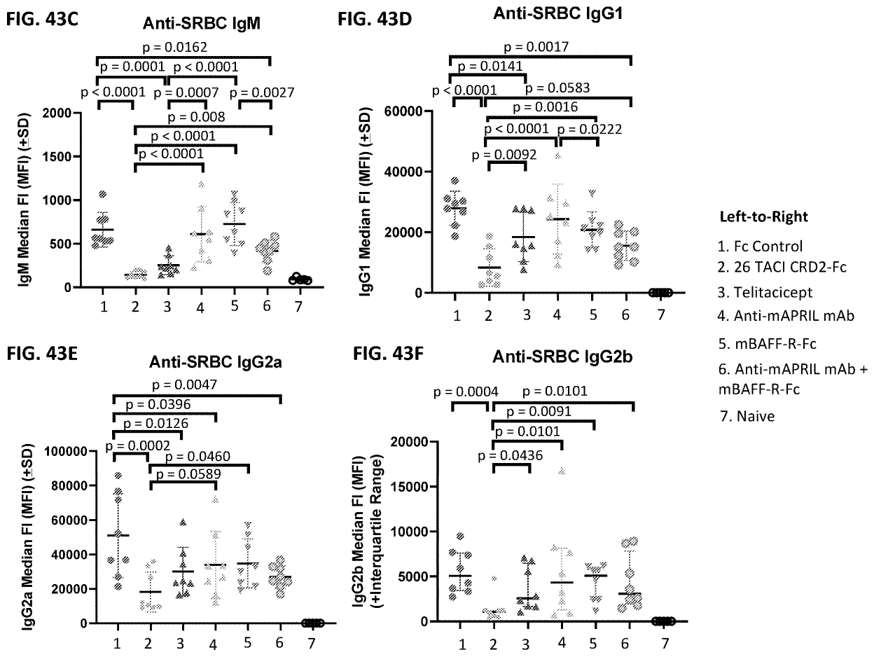

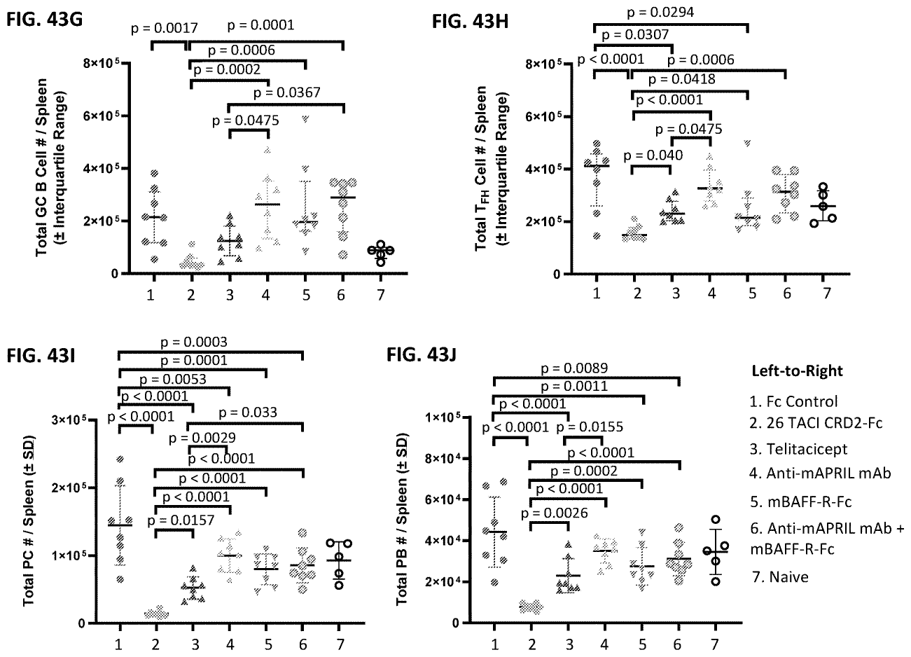

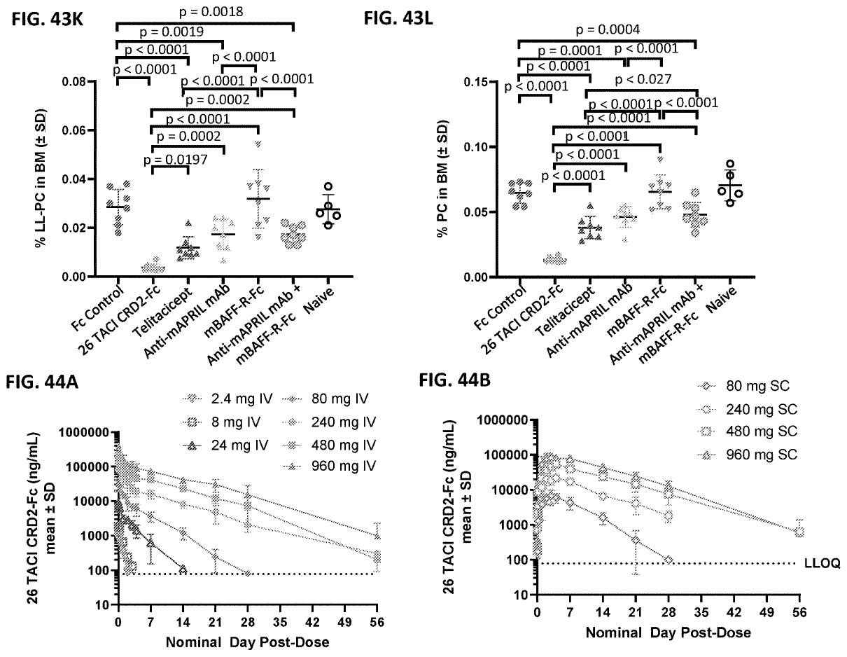

FIG. 43 shows that 26 TACI CRD2-Fc reduced serum anti-SRBC IgG1 levels (FIG. 43A, see also FIG. 43D) and bone marrow plasma cell numbers (FIG. 43B) in mice. The effect of the inhibition was superior to single anti-cytokine and combination biological therapy, including compared dual inhibitors of BAFF and APRIL (e.g. “Anti-APRIL+BAFF-R-Fc”).

26 TACI CRD2-Fc demonstrated significantly enhanced immunosuppressive activity over all of the comparators, including the combination of anti-mAPRIL+mBAFF-R-Fc, for most of the endpoints, including reduced anti-SRBC Ig responses (FIGS. 43C-43F), decreased numbers of splenic GC B cells (FIG. 43G), T_{FH} cells (FIG. 43H), PC (FIG. 43I), and plasmablasts (FIG. 43J), as well as reductions in the percent of LL-PC (FIG. 43K) and total PC in the bone marrow (FIG. 43L).

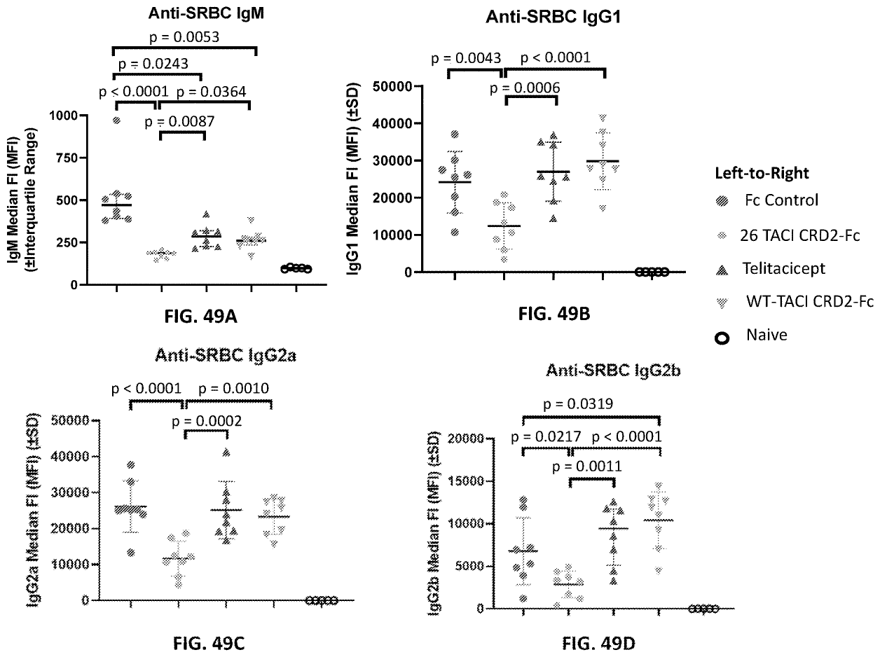

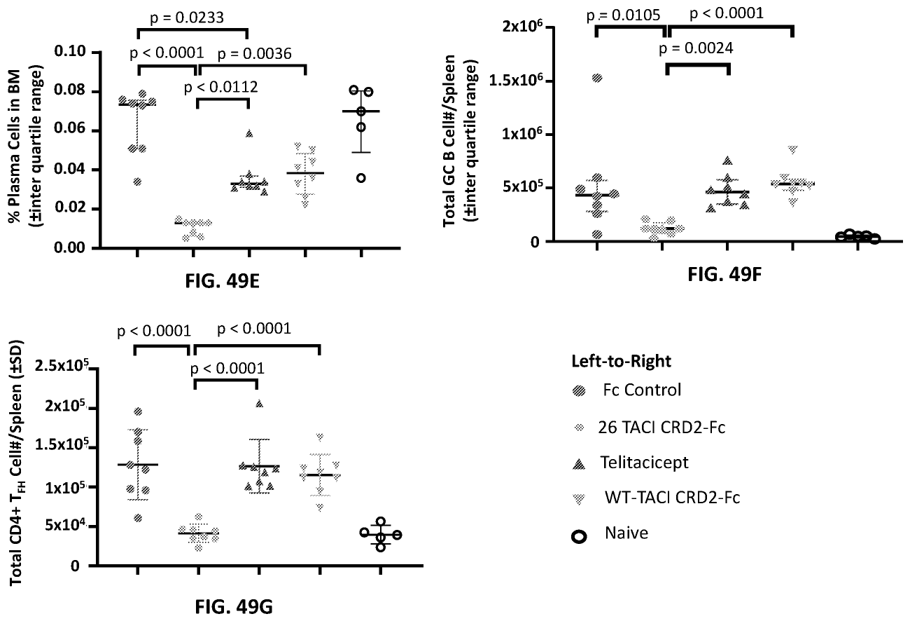

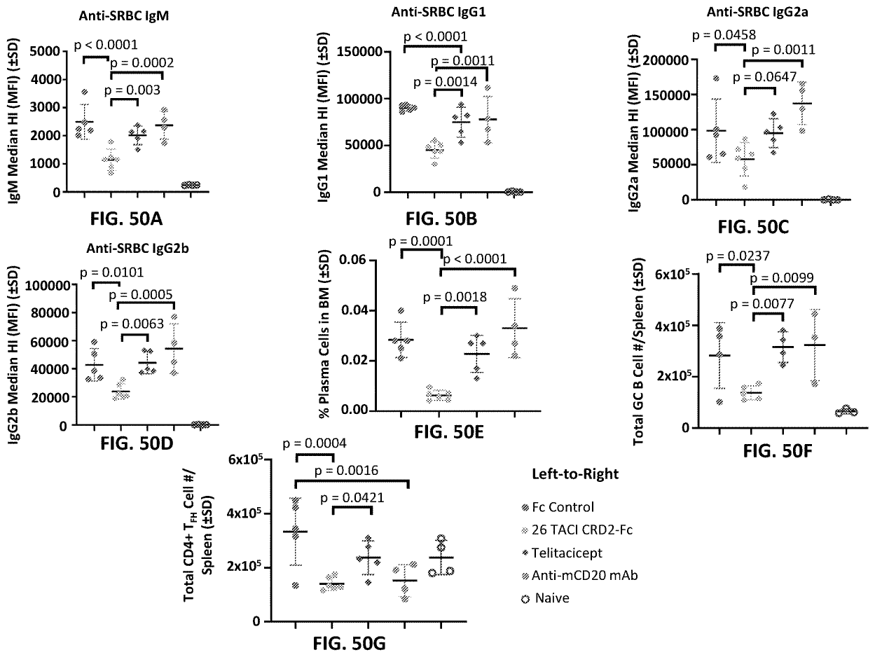

Similar SRBC immunization studies were conducted to compare the activity of 26 TACI CRD2-Fc to WT TACI CRD2-Fc as well as telitacicept and a depleting mouse anti-CD20 antibody. 26 TACI CRD2-Fc again demonstrated enhanced immunosuppressive activity over WT TACI CRD2-Fc, telitacicept and anti-CD20 as determined by endpoints similar to those listed above (FIGS. 49A-49G, FIGS. 50A-50G). That is, anti-SRBC IgM, IgG1, IgG2a and IgG2b were significantly reduced (FIGS. 49A-49D and FIGS. 50A-50D) and plasma cells, GC B cells and T_{FH} cells were significantly reduced (FIGS. 49E-49G and FIGS. 50E-50F). Collectively, the results from these immunization studies demonstrate that the optimized dual inhibition of APRIL and BAFF by 26 TACI CRD2-Fc provides deeper and more sustained ASC and B cell suppression as compared to anti-CD20, telitacicept, WT TACI CRD2-Fc, inhibitors of either APRIL or BAFF alone, or a combination of APRIL and BAFF inhibitors, leading to greater suppression of T cell-dependent antibody responses.

### Example 21. Administration of TACI CRD2-Fc in Healthy Subjects

Healthy adult subjects included in a clinical study (n=66) were administered a single dose of the exemplary TACI CRD2-Fc, 26 TACI CRD2-Fc (set forth in SEQ ID NO: 167). Safety, tolerability, pharmacokinetics (PK), and pharmacodynamics (PD) of the fusion protein were assessed. The 26 TACI CRD2-Fc product was provided as a 100 mg/mL liquid formulation with the following excipients: acetate, proline and polysorbate 80. The 26 TACI CRD2-Fc was provided as a single-use 2 mL glass vial with extractable volume of about 0.8 mL (80 mg). Before use, the 26 TACI CRD2-Fc product was stored at −20° C., protected from light.

### A. Study Design

Sixty-six healthy subjects (ages 18-65 years) were divided into 7 intravenous (IV) cohorts and 4 subcutaneous (SC) cohorts with 6 participants per cohort. The demographics of study participants is set forth in Table E39.

TABLE E39
Demographics and Disposition

| | Subjects | |
|---|---|---|
| Attribute | (N = 66) | |
| Age (year)[1] | 32 | (18, 64) |
| Female | 46 | (70%) |
| Race | 40 | (61%) |
| White | 13 | (20%) |
| Asian | 13 | (20%) |
| Other | | |
| BMI (kg/m2)[1] | 25 | (18, 31) |
| eGFR[2] | | |
| ≥90 | 53 | (80%) |
| 60-89 | 13 | (20%) |
| Completed[3] | 39 | (59%) |

[1]Median (min, max)
[2]mL/min/1.73 m2
[3]As of 29 Jul. 2022
BMI = body mass index;
eGRF = estimated glomerular filtration

The dosing protocol was based on the predicted human PK of 26 TACI CRD2-Fc using PK modeling and allometric scaling from PK data in cynomolgus monkeys, and a safety margin for the predicted human exposure based on the no-observed-adverse-effect level (NOAEL) toxicology studies in rats and monkeys, as described in Example 11 and 12.

For each IV cohort, the subjects were randomized 1:1 to receive a single IV dose (2.4 mg) of 26 TACI CRD2-Fc or placebo (normal saline, 0.9% w/v NaCl as a sterile solution) on Day 1. The planned starting dose of 2.4 mg IV is the minimum anticipated biological effect level (MABEL) based on the potential for hypercytokinemia as assessed in an in vitro cytokine release assay for TNFalpha. After approximately 24 hours of observation from the end of dosing, the remaining 4 participants in the IV cohort were randomized 3:1 to receive 26 TACI CRD2-Fc or placebo, respectively. After the last subject was dosed in the IV cohort at the first dose level, escalation to the next dose level proceeded following review of safety data. Based on the modeling and simulation data described above, subjects in the IV cohort were administered a single intravenous infusion over approximately 30 minutes of one of the following dose levels: 2.4 mg, 8 mg, 24 mg, 80 mg, 240 mg, 480 mg, and 960 mg. The starting dose of 2.4 mg IV is 1,780-fold and 923-fold lower than the human equivalent dose (HED) of the NOAEL in monkeys and rats, respectively. At a dose of 2.4 mg IV, the predicted human Cmax and area under the concentration-time curve (AUC) is 5,430-fold and 2,980-fold lower, respectively, than the Cmax and AUC observed at the NOAEL of 150 mg/kg in monkeys. The highest dose of 960 mg IV is 4.5-fold and 2.3-fold lower than the HED of the NOAEL in monkeys and rats, respectively. At 960 mg IV, the predicted human Cmax and AUC is 14-fold and 7.5-fold lower than the Cmax and AUC observed at the NOAEL of 150 mg/kg in monkeys.

For each SC cohort, subjects were randomized 4:2 to receive a single SC dose (80 mg) of 26 TACI CRD2-Fc or placebo, respectively, on Day 1. The subjects in the subcutaneous cohort were administered a single dose of one of the following doses levels or placebo: 80 mg, 240 mg, 480 mg, and 960 mg.

Baseline assessments were performed before the dose on Day 1. After dosing, the subjects were followed for safety and PK/PD until Day 113, or later if hypogammaglobulinemia has persisted longer. Subjects with quantitative immunoglobulin G (IgG) that was below the lower limit of normal at EOS were followed for assessment of quantitative Ig levels until there was evidence of recovery of Ig production. Safety was based on the incidence, severity and seriousness of adverse events, including clinically significant changes in physical exam findings, vital signs, laboratory tests (hematology, serum chemistry, coagulation, and urinalysis), and electrocardiograms.

Serum concentrations of 26 TACI CRD2-Fc were measured over time and PK endpoints were estimated, including maximum observed concentration (Cmax), time to maximum observed concentration (tmax), area under the concentration-time curve (AUC), and bioavailability of SC dosing. PD endpoints were measured and included (1) Serum IgM, IgG (total, IgG1, IgG2, IgG2 and IgG4), IgA (total, IgAQ1 and IgA2), and IgE levels, and their corresponding changes from baseline over time; and (2) circulating B cell populations, including antibody-secreting cells (ASCs). The incidence of anti-drug antibodies (ADA), time to first ADA, and titer of ADA against 26 TACI CRD2-Fc was assessed. Exploratory endpoints, including circulating B and T lymphocytes including their subtypes (such as transitional B cells, follicular B cells, marginal zone B cells, plasmablasts and plasma cells), mean serum levels and changes from baseline over time in relevant circulating biomarkers were measured.

### B. Results

No treatment-related serious adverse events, infusion-related reactions, or adverse trends in safety laboratories were reported in any of the dosed cohorts. Results demonstrated that the exemplary 26 TACI CRD2-Fc was well tolerated in all IV and SC cohorts (Table E40A and Table E40B). The most common adverse events (AEs) included mild (G1) headache, dizziness, low Ig (an expected pharmacodynamics (PD) effect), or back pain. Incidence of infection did not significantly differ from placebo. There were no observed grade 4 or 5 (G4-5) AEs, serious AEs, serious or severe infection, severe hypogammaglobulinemia, or cytokine release (no significant changes in: GM-CSF, IFN-γ, IL-3, IL-5, IL-6, IL-7, IL-8, IL-10, IL-18, MIP-1α, MIP-1β, MCP-1, TNF-α, or TNF-β).

TABLE E40A

| | | |
|---|---|---|
| | | All 26 |
| | All | TACI |
| Treatment-Emergent Adverse | Placebo | CRD2-Fc |
| Event (TEAE) | (N = 22) | (N = 44) |

| | | | | |
|---|---|---|---|---|
| Any TEAE^{[1]} | 12 | (55%) | 27 | (61%) |
| Grade 1 | 7 | (32%) | 20 | (45%) |
| Grade 2 | 4 | (18%)^{[2]} | 6 | (14%)^{[3]} |
| Grade 3 | 1 | (5%)^{[4]} | 1 | (2%)^{[4]} |
| Adverse Event of Interest | 1 | (5%) | 1 | (2%) |
| Administration-Related Reaction^{[5]} | 1 | (5%) | 1 | (2%) |
| Injection Site Pain (Grade 1) | 1 | (5%) | 1 | (2%) |

| | | |
|---|---|---|
| Serious or Severe Infection | 0 | 0 |
| Severe Hypogammaglobulinemia | 0 | 0 |
| Cytokine Release Syndrome | 0 | 0 |

^{[1]}Follow-up through day 29 post-dose
^{[2]}Upper respiratory tract infection, hyperlipasaemia, dyspepsia, fatigue and iron deficiency anaemia (n = 1 each)
^{[3]}Urinary tract infection, headache, migraine, nausea, and back pain (n = 1 each); lipase increased (n = 2)
^{[4]}Blood creatine phosphokinase increased, attributed to strenuous exercise
^{[5]}Infusion-related reaction, injection-related reaction, injection site pain, or injection site reaction

TABLE E40B
Most Common^{[1]} Treatment-Emergent Adverse Events (TEAEs).

| | | | All 26 | |
|---|---|---|---|---|
| | All | | TACI | |
| Preferred Term | Placebo | | CRD2-Fc | |
| (Any Grade) | (N = 22) | | (N = 44) | |
| Headache or Migraine | 4 | (18%) | 11 | (25%) |
| Grade 1 | 4 | (18%) | 9 | (20%) |

| | | | |
|---|---|---|---|
| Grade 2 | 0 | 2 | (5%) |

| | | | | |
|---|---|---|---|---|
| Infections^{[2]} | 4 | (18%) | 6 | (14%) |
| Grade 1 | 3 | (14%) | 5 | (11%) |
| Grade 2 | 1 | (5%) | 1 | (2%) |
| Dizziness^{[3]} | 1 | (5%) | 5 | (11%) |
| Grade 1 | 1 | (5%) | 4 | (9%) |

| | | | |
|---|---|---|---|
| Grade 2 | 0 | 1 | (2%) |
| Hypogammaglobulinemia^{[4]} | 0 | 4 | (9%) |
| Grade 1 | 0 | 4 | (9%) |

^{[1]}Experienced by more than 5% of subjects treated with 26 TACI CRD2-Fc
^{[2]}Events coded in system organ class of Infections and Infestations including COVID-19, nasopharyngitis, viral URTI (all G1); URTI (G2) in subjects in the placebo group and COVID-19 (n = 2), URTI (n = 2), furuncle (all G1); urinary tract infection (G2) in subjects treated with 26 TACI CRD2-Fc
^{[3]}‘Dizziness,’ ‘dizziness postural’ or ‘presyncope’
^{[4]}‘Blood immunoglobulin M decreased’ or ‘hypogammaglobulinemia’
G1 = Grade 1;
G2 = Grade 2;
URTI = upper respiratory tract infection

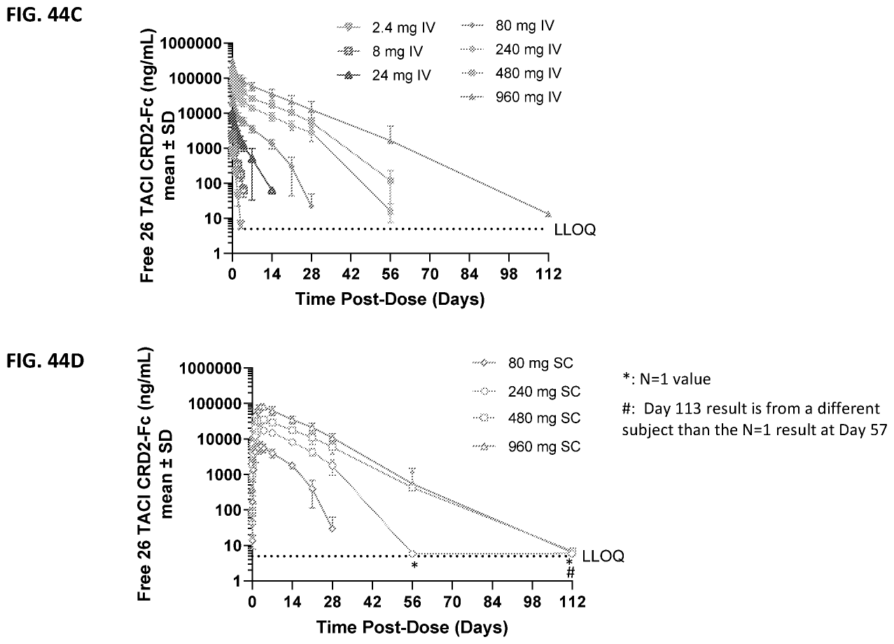

All cohorts exhibited dose-dependent PK and expected PD effects on circulating Ig levels, including reductions in serum Ig starting at 8 mg IV (~0.1 mg/kg). Dose-dependent PK was observed by both IV (FIG. 44A, see also FIG. 44C) and SC (FIG. 44B, see also FIG. 44D) administration. The 240 mg SC dose demonstrated a bioavailability of −63% and a half-life of about 7.5 days (CV=24%) as assessed at post-dose time points up to 28 days or a bioavailability of −75% and a half-life of about 7.4 days as assessed at post-dose time points up to 56 days. The 80 mg SC dose demonstrated a bioavailability of >100% and a half-life of about 3.1 as assessed at post-dose time points up to 28 days or a bioavailability of −70% and a half-life of about 3.7 days as assessed at post-dose time points up to 56 days. Overall, good bioavailability of 70 to 81% was observed at 80 to 960 mg.

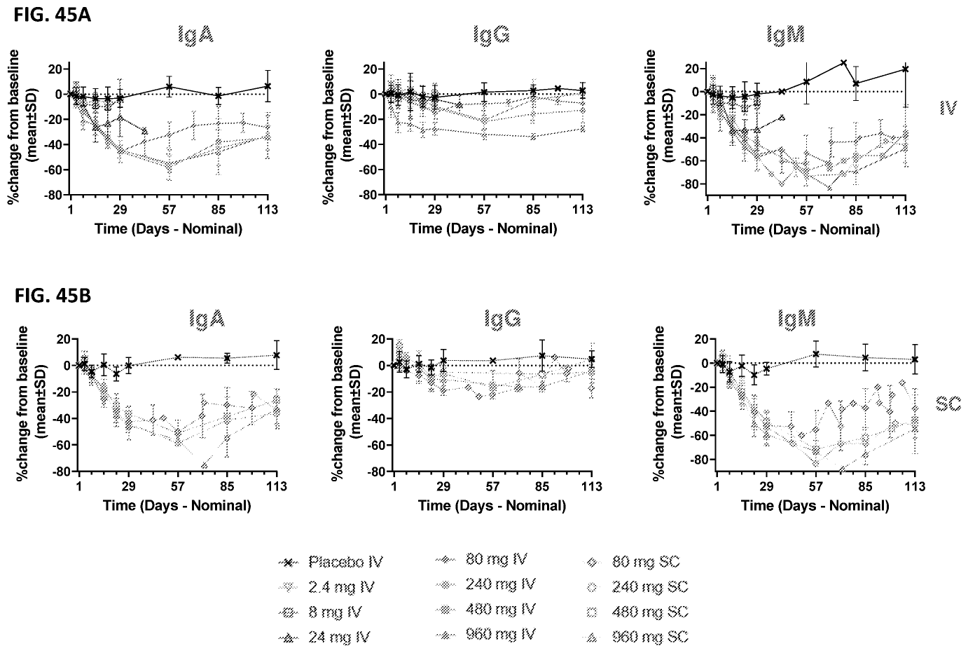

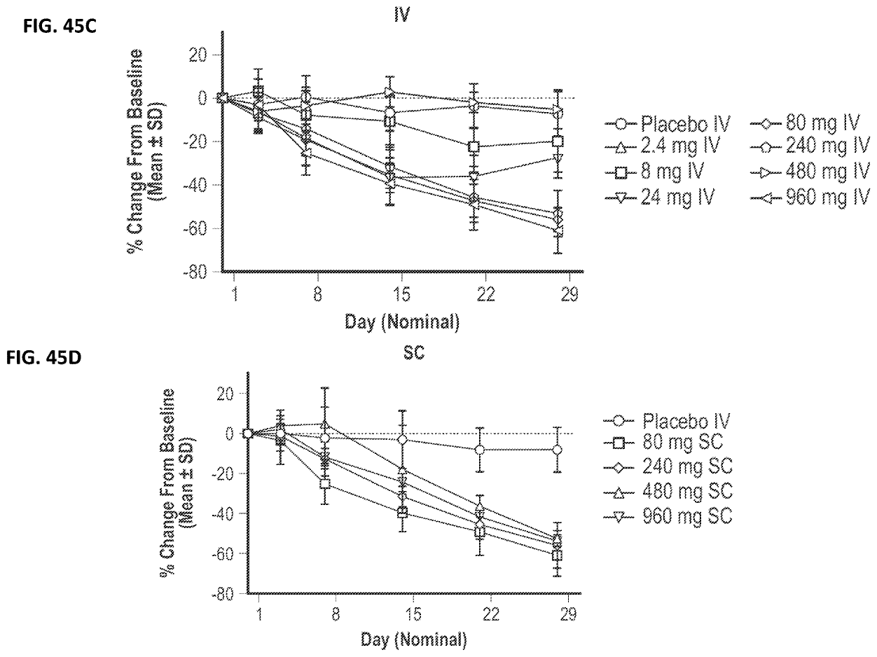

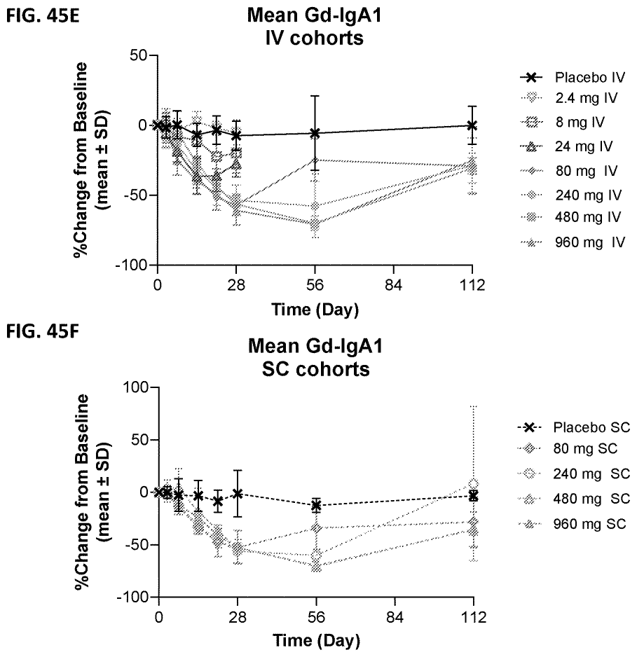

26 TACI CRD2-Fc decreased circulating immunoglobulins in human subjects who received IV or SC administration. Serum IgA, IgG, IgM levels, and their corresponding changes from baseline over time as were measured in subjects that received IV administration of 26 TACI CRD2-Fc is shown in (FIG. 45A). Serum IgA, IgG, IgM levels, and their corresponding changes from baseline over time as measured in subjects that received SC administration of 26 TACI CRD2-Fc is shown in FIG. 45B. Dose-dependent, on-target reductions in circulating immunoglobulins was shown for IgM>IgA>IgG. A maximal negative slope was observed at doses ≥80 mg for ≥4 weeks, suggesting saturated PD. Post-treatment nadirs<lower limit of normal (LLN) generally were more frequently observed among participants whose baselines were in the lowest quintile (0.5-0.92 g/L for IgM) of the normal range. Post-dose reductions in IgM<LLN (0.5 g/L) were observed in 26/50 (52%) versus 1/22 (5%) of 26 TACI CRD2-Fc-versus placebo-treated participants, respectively. No incidences of total IgG≤LLN (6.1 g/L) were observed (FIG. 45A and FIG. 45B). A statistical relationship between dose and incidence of post-treatment Ig reductions <LLN could not be confirmed.

Serum galactose-deficient IgAQ1 (Gd-IgA1) levels and its corresponding changes from baseline over time was measured in subjects that received IV (FIG. 45C, see also FIG. 45E) or SC administration of 26 TACI CRD2-Fc is shown in (FIG. 45D, see also FIG. 45F).

The enhanced PK and immunomodulatory properties of 26 TACI CRD2-Fc vs. WT TACI-Fc is consistent the use of lower and/or less frequent doses of 26 TACI CRD2-Fc in humans. The effect of SC administration of 80 mg 26 TACI CRD2-Fc on circulating immunoglobulins was compared to published data on administration of comparator molecules, including 150 mg Atacicept (WT TACI 30-110) administered SC (Willen 2020 Eur J Drug Metabl Pharmacokinet 45:27-40), 160 mg Telitacicept (WT TACI 13-118-Fc) administered SC (Xie 2022 Clin Pharmacol Drug Dev. 10.1002/cpdd.1136), 450 mg or 1350 mg anti-APRIL antibody BION-1301 administered IV (Lo 2020 Kidney Week Abstract P)1843 (ASN 31:2020)), or 12 mg/kg humanized IgG2 anti-APRIL antibody Sibeprenlimab administered IV (Mathur 2022 Kidney Int Rep 7:993-1003). FIG. 46 shows that administration of 80 mg 26 TACI CRD2-Fc SC compares favorably to the other molecules for decreasing circulating immunoglobulins, indicating efficacy after SC administration and at the lower 80 mg dose. The results are consistent with the relevance of APRIL>BAFF to circulating immunoglobulins in healthy adults. Further, the results indicate that 26 TACI CRD2-Fc exhibits improved APRIL activity versus WT TACI-Fc molecules.

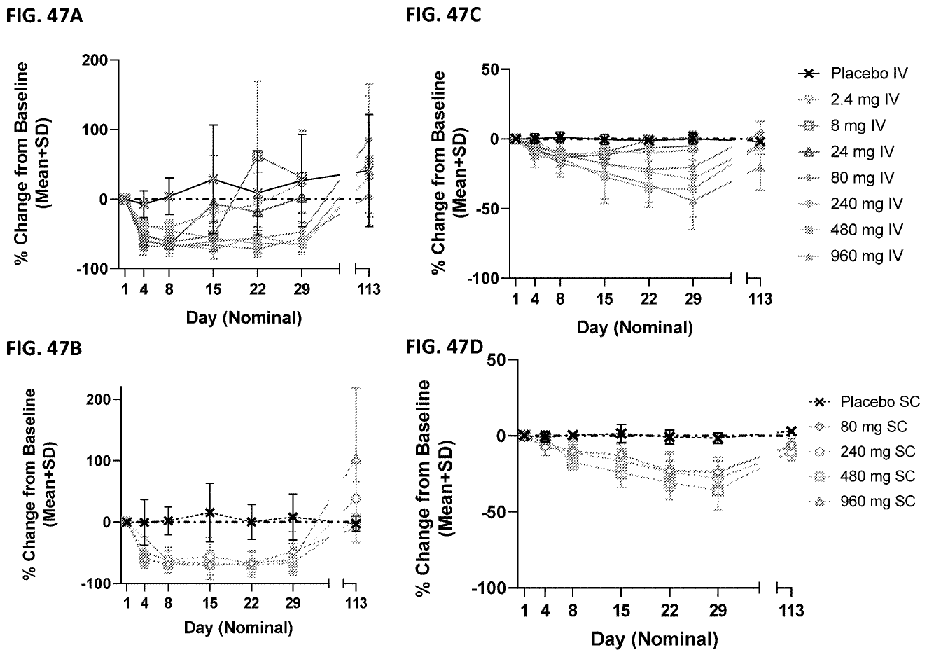

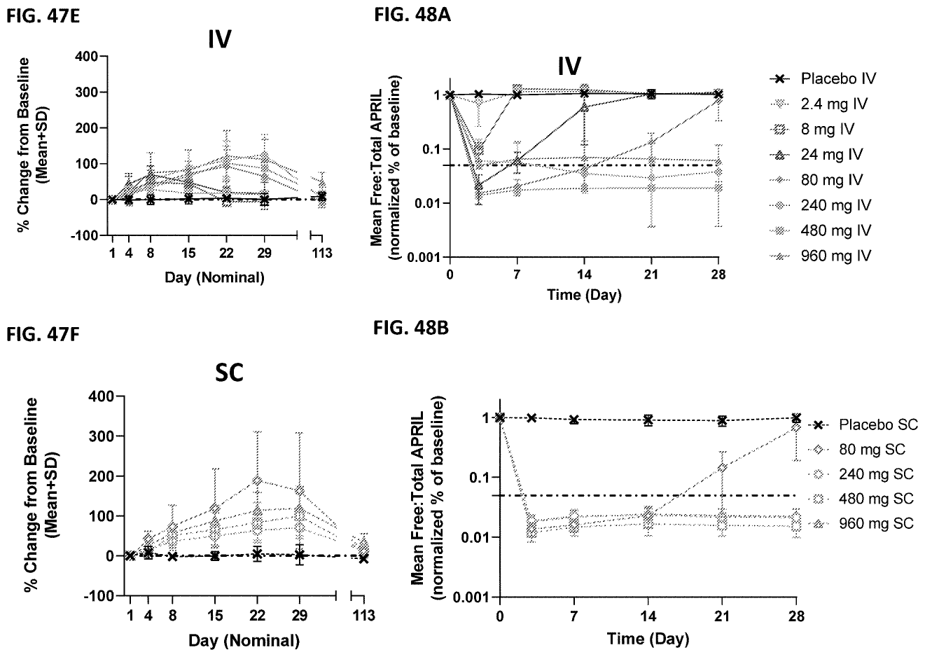

Circulating Antibody-Secreting cells (ASCs), CD19+CD38+CD27+IgD− cells, were also measured in treated subjects by determining the frequency of CD38^{hi} plasmablasts & plasma cells, of gated CD19+CD27+IgD− memory B cells. Dose-dependent, on target reductions in the frequency of circulating CD19+CD38+CD27+IgD− ASCs was observed after IV (FIG. 47A) or SC (FIG. 47B) administration. Maximal reductions were observed at doses ≥80 mg, suggesting saturated PD.

Circulating Naïve B cells, CD27−IgD+ cells, were also measured in treated subjects by determining the frequency of CD27−IgD+ cells of gated CD19+ cells. Dose-dependent, on target reductions in the frequency of circulating Naïve B cells was observed after IV (FIG. 47C) and SC (FIG. 47D) administration.

Circulating Memory B cells, CD27+IgD− cells, were also measured in treated subjects by determining the frequency of CD27+IgD− cells of gated CD19+ cells. As shown in FIG. 47E and FIG. 47F, dose-related increases in the frequency of circulating memory B cells were observed. Dose-dependent, on target reductions in the frequency of circulating Memory B cells were also observed after IV (FIG. 47E) and SC (FIG. 47F) administration.

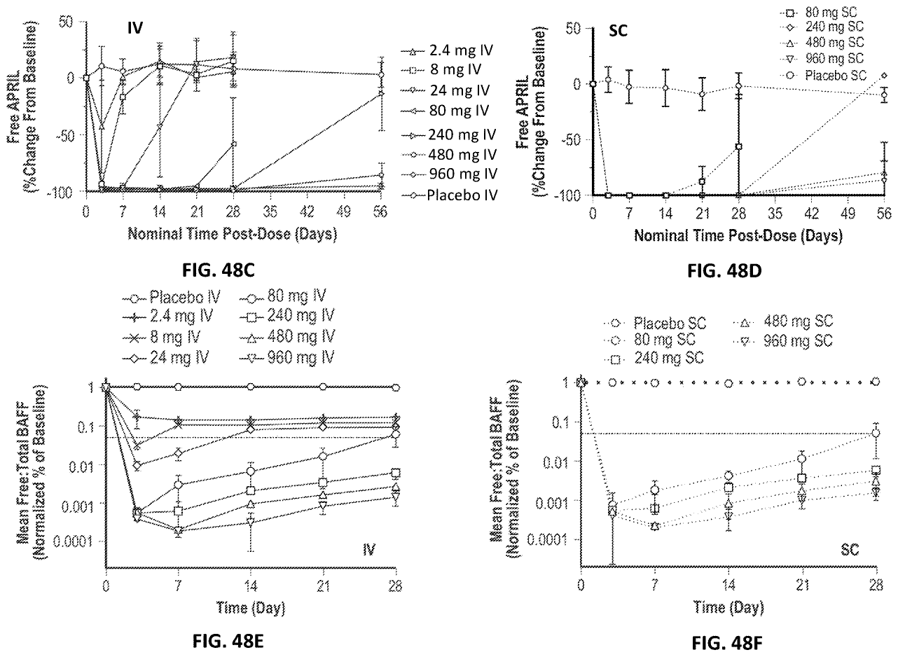

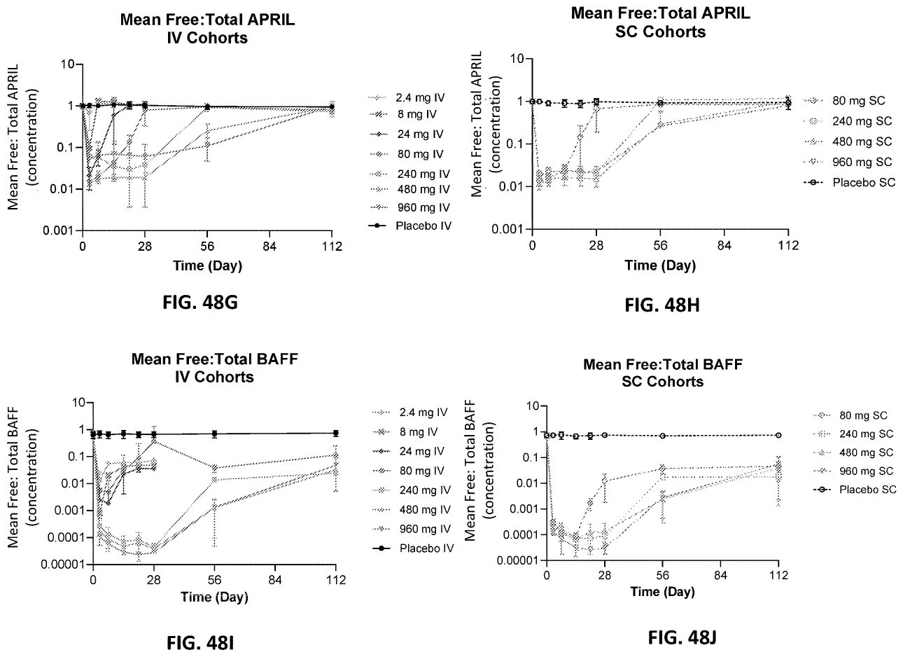

Administration of 26 TACI CRD2-Fc also demonstrated dose-dependent reductions in circulating free APRIL through day 28 post-dose after intravenous administration (FIG. 48A) and subcutaneous administration (FIG. 48B). Through Day 28 post-dose, there was >95% coverage achieved by both IV and SC administration of 26 TACI CRD2-Fc at 240 mg. The dose-dependent reductions in circulating free APRIL was further observed through day 56 post-dose after intravenous administration (FIG. 48C, see also FIG. 48G) and subcutaneous administration (FIG. 48D, see also FIG. 48H). Administration of 26 TACI CRD2-Fc also demonstrated dose-dependent reductions in circulating free BAFF through day 28 post-dose after intravenous administration (FIG. 48E, see also FIG. 48I) and subcutaneous administration (FIG. 48F, see also FIG. 48J).

Thus, the results showed that the coverage of free APRIL was maintained for ≥4 weeks with a 240 mg dose IV or SC, and 2-3 weeks with an 80 mg dose IV or SC, which also corresponded to reduction in serum Ig and ASCs as demonstrated above. Additionally, >95% coverage of APRIL and BAFF was achieved through Day 28 post IV and SC administration of the 240 mg dose. These data support dose regimens of 80-240 mg SC every 4 weeks in further studies.

Results from the study indicate that the exemplary 26 TACI CRD2-Fc demonstrates acceptable preliminary safety and tolerability, and exhibits PD effects on circulating Ig and B cell populations. These results are based on comparison to activity of WT TACI-Fc molecules in healthy volunteers and further demonstrate superiority against CD20 depleting antibodies as well as WT TACI-Fc and inhibitors of BAFF and/or APRIL alone. These findings support future clinical development of exemplary 26 TACI CRD2-Fc in patients with SLE and/or other B cell- and/or autoantibody-related diseases. For example, among other diseases, the results demonstrating dose-dependent reduction in Ig levels, including galactose-deficient IgAQ1 (Gd-IgAQ1) and circulating Ab-secreting cells (ASCs), suggest significant potential in the treatment of Autoantibody-Associated Glomerulonephritis (GN).

### Example 22. Administration of TACI-Fc Fusion Protein in Subjects with Autoantibody-Associated Glomerular Disease

Adults with a diagnosis of autoantibody-associated glomerular disease are administered a composition containing TACI vTD-Fc fusion protein designated 26 TACI CRD2-Fc (Fc fusion protein set forth in SEQ ID NO:167). The TACI vTD-Fc fusion protein composition is formulated as a 100 mg/mL liquid in single-use glass vials with extractable volumes of 0.8 mL/vial (80 mg per vial). Safety and response to administration of the TACI vTD-Fc fusion protein are assessed. The study is part of an ongoing clinical trial study.

### Subjects and Treatment

A group of adult human subjects are selected for administration of a dose of TACI vTD-Fc fusion protein in one of 3 ascending dose cohorts. The subjects are those with a diagnosis of autoantibody-associated glomerular disease, including immunoglobulin (Ig) A nephropathy (IgAN), lupus nephritis (LN), primary membranous nephropathy (pMN), or renal anti-neutrophil cytoplasmic antibody (ANCA)-associated vasculitis (AAV) are enrolled.

The inclusion criteria for the subjects includes autoantibody-associated glomerular disease of one of the following types: a) Immunoglobulin (Ig) A nephropathy (IgAN), with i) a biopsy-confirmed diagnosis within ≤3 years prior to the start of screening, and ii) elevated galactose deficient IgAQ1 (Gd-IgAQ1) antibodies at screening; b) Lupus nephritis (LN), with i) biopsy-confirmed diagnosis within ≤1 year prior to the start of screening (renal biopsies showing evidence of active, proliferative class III or IV LN per the international society of nephrology/renal pathology society (ISN/RPS) criteria; subjects may co-exhibit class V disease in addition to either class III or class IV disease), ii) elevated anti-double stranded DNA (anti-dsDNA) at screening, and iii) positive for anti-nuclear antibody (ANA) with titers of ≥1:80 at screening; c) primary membranous nephropathy (pMN), with i) biopsy-confirmed diagnosis within ≤3 years prior to the start of screening, and ii) positive for anti-phospholipase A2 receptor (anti-PLA2R1) antibodies and/or anti-thrombospondin type-1 domain-containing 7A (anti-THSD7A) antibodies at screening; and d) renal anti-neutrophil cytoplasmic antibody (ANCA)-associated vasculitis (AAV), with i) biopsy-confirmed diagnosis within ≤2 years prior to the start of screening with evidence of renal ANCA-associated vasculitis, ii) positive for anti-proteinase 3 (PR3) or anti-myeloperoxidase (MPO) antibodies at screening, and iii) sustained immunological activity as evidence of well-documented positive PR3 or MPO antibodies within ≤6 months prior to the start of screening (the minimum period between screening and historical ANCA result is ≥14 days). In any of the above types, if a biopsy is not performed within the specified timeframe or a report is not available, a biopsy is performed during screening after having met all other eligibility criteria. Additional inclusion criteria include 1) sustained proteinuria, measured as urine total protein/creatinine ratio (UPCR)≥0.75 g/g, with the first assessment determined using either 24-hour urine or spot urine collection (visit 1) and a the second assessment (≥14±3 days later at visit 2) determined using 24-hour urine collection; and 2) resting systolic blood pressure <150 mm Hg and resting diastolic blood pressure <90 mm Hg.

The adult human subject exclusion criteria include prior diagnosis of, or a diagnostic criteria for another renal disease including but not limited to diabetic nephropathy, C3 glomerulonephropathy, focal segmented glomerulosclerosis, thin basement membrane disease, Alport's disease, IgA vasculitis, minimal change disease, post-infectious glomerulonephritis, secondary membranous nephropathy (excluding LN class V combined with class II or IV) or secondary IgAN including but not limited to Celiac disease, Crohn's disease, HIV, or liver cirrhosis; previous treatment history of the following within the described period prior to Day 1: rituximab or other agents that directly deplete B lymphocytes (48 weeks), Belimumbab or other agents that directly inhibit B cell activating factor (BAFF) and/or a proliferation inducing ligand (APRIL) (24 weeks), intravenous Ig, abatacept, anifrolumab, belatacept, adalimumab, inflizimab, certolizumab, etanercept, golimumab, anakinra, canakinumab, tocilizumab, sarilumab, satralizumab, or other marketed biological therapeutics (8 weeks), cyclophosphamide (8 weeks), any non-biological investigational agent (8 weeks or 5 half-lives), and other biological investigational agents (5 half-lives). Other factors of exclusion are within the level of a skilled clinician or physician.

Subjects are administered a subcutaneous injection of TACI vTD-Fc fusion protein at a dose of 80 mg, 160 mg, or 240 mg once every 2 weeks (Q2W). An intravenous infusion of a may be considered once every 4 weeks (Q4W). The treatment period continues for up to 48 weeks. In some cases, subjects may be administered the dose (e.g. 80 mg) every other week (Q2W), or a lower dose once weekly (Q1W,) for 3-4 doses, and then may be administered that dose or a higher dose Q4W for the treatment period. After treatment, subjects may be monitored such as for safety.

### Safety and Efficacy Endpoints

Incidences of Treatment Emergent Adverse Events (TEAEs), Severe Adverse Events (SAEs), and adverse events of interest, dose-limiting toxicities, and treatment-emergent clinically significant abnormalities are monitored.

Immunological responses including, change from baseline over time in circulating levels of anti-dsDNA in subjects with LN, galactose deficient (Gd)IgA1 and anti-Gd-IgAQ1 in subjects with IgAN, and anti-PLA2R1 and anti-THSD7A in subjects with pMN, and anti-MPO, anti-PR-3 in subjects with renal AAV are monitored. Changes from baseline overtime of complement components (C3, C4, CH50) also are monitored.

Subjects also are monitored for one or more of immunological indices associated with disease activity of subjects with autoantibody-associated glomerular diseases; efficacy of TACI-Fc fusion protein assessed by changes from baseline over time in proteinuria, estimated glomerular filtration rate (eGFR), and associated composite renal function endpoints; and assessment of immunogenicity, pharmacokinetics, pharmacodynamics of TACI-Fc fusion protein in the adult subjects.

Clinical responses that are monitored in subjects also include change from baseline over time in urine protein:creatinine ratio (UPCR) assessed as 24-hour urine and/or spot urine; changes from baseline over time of estimated glomerular filtration rate (eGFR; e.g. calculated based on the cystatin C, race-independent equation described in Inker, 2021 and Chronic Kidney Disease Epidemiology Collaboration, CKD-EPI, equation); renal response at weeks 24 and 48 (LN and pMN subjects only) for determination of eGFR using cystatin C, race-independent equation (Inker, 2021); changes from baseline over time in physician's global assessment (PGA), and changes from baseline over time in patient's global assessment (PtGA). For LN subjects, changes in baseline over time in SLE disease activity indices (e.g. hybrid SELENA-SLEDAI and SLICC damage index scores) also are monitored. For renal AAV subjects, changes from baseline over time in AAV disease activity indices (e.g. using Birmingham Vasculitis Activity Score (BVAS) and Vasculitis Damage Index (VDI) scores) are monitored.

Pharmacokinetic (PK) and Pharmacodynamic (PD) endpoints are assessed at the administered doses. Pharmacodynamics (PD) endpoints include changes from baseline over time in serum Ig isotypes (IgM, IgA, total IgG, IgG1, IgG2, IgG3, IgG4, and IgGE), and in peripheral blood lymphocytes and subsets are assessed. In addition, changes in biomarkers related to renal inflammation renal inflammation/damage, lupus activity, and immune pathways mediated through soluble analytes (e.g., BAFF, APRIL, sTACI, sBCMA, sBAFF-R) are monitored. Pharmacokinetics (PK) endpoints including serum and urine levels of TACI-Fc fusion protein over time are estimated. Incidence and titers of anti-drug antibody (ADA) against TACI-Fc fusion protein are monitored.

### Example 23. Administration of TACI-Fc Fusion Protein in Subjects with Systemic Lupus Erythematosus (SLE)

Adults with a diagnosis of systemic Lupus Erythematosus are administered a composition containing TACI vTD-Fc fusion protein designated 26 TACI CRD2-Fc (Fc fusion protein set forth in SEQ ID NO:167). Safety, efficacy, immunogenicity, pharmacokinetic and pharmacodynamic response to administration of the TACI vTD-Fc fusion protein are assessed. The study is part of an ongoing clinical trial study.

A group of adult human subjects (n=270) are selected for administration of 26 TACI CRD2-Fc fusion protein. The subjects are those with active SLE for ≥6 months. In this study, the subjects are selected as having moderate to severe SLE as defined by further inclusion criteria at screening that includes: subjects with either a hybrid SELENA-SLEDAI score ≥8 or ≥6, in addition to high anti-dsDNA or low complement (C) levels (i.e. C3 or C4 levels); and subjects with a BILAG-2004 of level A in ≥1 organ system or level B in ≥2 organ systems; and subjects with a PGA≥1.0. Inclusion criteria also includes ≤6 g/g urine total protein to creatinine ratio (proteinuria). Subjects also include those receiving at least one stable standard-of-care background therapy (e.g. hydroxychloroquine, oral immunosuppression). Among exclusion criteria is recent severe nephritis, neuropsychiatric lupus, and comorbidities that may affect disease assessments.

Subjects are administered a subcutaneous (SC) injection of 26 TACI CRD2-Fc fusion protein at 80 mg SC Q4W or 240 mg SC Q4W or placebo.

Incidences of Treatment Emergent Adverse Events (TEAEs), Severe Adverse Events (SAEs), and adverse events of interest, dose-limiting toxicities, and treatment-emergent clinically significant abnormalities are monitored.

Disease activity is assessed. In some cases, disease activity is as determined by primary % SRI-4 at 48 weeks. An SRI-4 response is a composite endpoint defined by the following criteria: Reduction from baseline of ≥4 points in Systemic Lupus Erythematosus Disease Activity Index 2000 (SLEDAI-2K); No new organ system affected as defined by no new organ system with British Isles Lupus Assessment Group-2004 (BILAG-2004) grade A and no more than 1 new organ system with BILAG-2004 grade B compared with baseline; No worsening from baseline in lupus disease activity as defined by <0.3-point increase on 3-point Physician's Global Assessment (PGA)—Visual Analog Scale (VAS); and No violation to protocol-specified medication rules.

Other efficacy endpoints include flares; other derivatives of BILAG (BICLA), SLEDAI, PGA; Cutaneous LE Disease Area and Severity Index (CLASI); patient reported outcomes; and corticosteroid usage. Disease markers are measured, including anti-DNA, C3/C4 and urine protein:creatinine ratio (UPCR). Other biomarkers include CD50. Safety, PK, immunogenicity, and anti-drug antibody (ADA) also are assessed. PD is assessed by measurement of biomarkers, including soluble ligands (APRIL, BAFF); serum IgG, IgA and IgM; and circulating B cell subsets.

### Example 24. Administration of TACI-Fc Fusion Protein in Subjects with Glomerulonephritis, Including IgA Nephropathy, Lupus Nephritis and Primary Membranous Nephropathy

Adults with a diagnosis of active glomerulonephritis (GN), including active IgA nephropathy, IgAN), active lupus nephritis (LN) or active primary membranous nephropathy (PMN), were administered a composition containing TACI vTD-Fc fusion protein designated 26 TACI CRD2-Fc (Fc fusion protein set forth in SEQ ID NO:167). Safety and response to administration of the TACI vTD-Fc fusion protein were assessed. The study was part of a clinical trial study.

A group of adult human subjects were selected for administration of 26 TACI CRD2-Fc fusion protein. The subjects were those with:
(1) IgA Nephropathy (IgAN) with a confirmed diagnosis (e.g., by biopsy) within ≤5 years prior to the start of screening, characterized by proteinuria ≥0.5-0.75 g/g urine total protein to creatinine ratio (UPCR); and standard of care therapy, such as stable background immunosuppression;
(2) Lupus Nephritis (LN) that is LN Class III-V with a confirmed diagnosis (e.g., by biopsy) within ≤3 years prior to the start of screening and characterized by proteinuria ≥1 g/g urine total protein to creatinine ratio (UPCR); active urinary sediment; positive anti-DNA and antinuclear antibody (ANA) (such as wherein positive anti-dsDNA is a titer of ≥30 IU/mL and positive ANA is a titer of ≥1:80); and standard of care therapy, such as stable background immunosuppression; or
(3) Primary Membranous Nephropathy (pMN) with a confirmed diagnosis (e.g., by biopsy) within ≤5 years prior to the start of screening, less than 50% reduction of proteinuria in the last 24 weeks while on angiotensin-converting enzyme (ACE)/angiotensin receptor blocker (ARB), and characterized by proteinuria ≥3.5 g/g urine total protein to creatinine ratio (UPCR); positive anti-PLA2R or anti-THSD7A; and standard of care therapy, such as stable background immunosuppression. Further inclusion criteria for the subjects includes subjects having received maximal angiotensin-converting enzyme (ACE)/angiotensin receptor blocker (ARB) therapy for ≥12 weeks prior to study Day 1, stable blood pressure (BP) therapy, and no prohibited concomitant medications.

Exclusion criteria include prior diagnosis of, or fulfills diagnostic criteria for, another glomerular disease; eGFR<30 mL/min/1.73m2 or rapidly progressive glomerulonephritis; or recent serious or ongoing infection; risk or history of serious infection.

Additional or alternative inclusion criteria are shown in Table E41A and exclusion criteria are shown in Table E41B.

TABLE E41A
Inclusion Criteria for IgAN, pMN and LN

| IgAN | pMN | LN |
|---|---|---|
| No background | less than 50% reduction of | biopsy ≤24 weeks |
| immunosuppression | proteinuria in the last 24 | UPCR ≥3.5 g/g |
| | weeks while on | On maintenance |
| | angiotensin-converting | treatment (e.g., |
| | enzyme (ACE)/ | MMF, AZA, CnIs), |
| | angiotensin receptor | post induction |
| | blocker (ARB) | |
| | no background | |
| | immunosuppression, such | |
| | as stable background | |
| | immunosuppression, such | |
| | as wherein the | |
| | stable background | |
| | immunosuppression is a | |
| | stable dose of calcineurin | |
| | inhibitor (CnI) | |
| | for ≥12 weeks | |

Age ≥18 years
Stable maximal ACEI/ARB and other proteinuria-lowering medications ≥12 weeks
Intent to maintain any background immunosuppression through treatment period
Well-controlled blood pressure

TABLE E42B
Exclusion Criteria for ITP, wAIHA and CAD

| | | |
|---|---|---|
| IgAN | pMN | LN |

sclerosis or interstitial fibrosis and tubular atrophy in >50%
of the biopsy
receiving prior B cell depletion or BAFF/APRIL inhibitors
for ≤24 weeks
having a history of demyelinating disorder; and known
immunodeficiency, including IgG <700 mg/dL, IgA <10
mg/dL, B cells <100 per μL, or CD4 <200 per mm³

Subjects were administered a subcutaneous (SC) injection of 26 TACI CRD2-Fc fusion protein at 80 mg SC Q4W or 240 mg SC Q4W. Primary treatment was for 24 weeks, with an optional 24 week extension. Extension criteria include that the subjects had no grade ≥2 Treatment Emergent Adverse Events (TEAEs), the treatment demonstrated clinical benefit (e.g. UPCR improvement >25%), the subject met all applicable eligibility criteria, and medical monitor approval was gained. A dose escalation >240 mg was considered if there is high drug clearance due to proteinuria.

Incidences of Treatment Emergent Adverse Events (TEAEs), Severe Adverse Events (SAEs), and adverse events of interest, dose-limiting toxicities, and treatment-emergent clinically significant abnormalities were monitored.

Study endpoints included change in disease-related antibodies: Gd-IgA1 and anti-Gd-IgAQ1 (IgAN), anti-DNA such as anti-dsDNA (LN), or anti-PLA2R1 or anti-THSD7A (pMN). Study endpoints also include change in biomarkers of the complement system including complement component 3 (C3), complement component 4 (C4), or total hemolytic complement (CH50). Study endpoints further include proteinuria and eGFR. Study endpoint also include measuring disease activity. Disease activity was determined by urine protein:creatinine ratio (UPCR) (for example, up to ~20 wks), eGFR, renal response and corticosteroid usage. Safety, PK and anti-drug antibody (ADA) also were assessed. PD was assessed by measurement of biomarkers, including soluble ligands (APRIL, BAFF); serum IgG, IgA and IgM; and circulating B cell subsets.

Patients with IgAN, pMN, and LN were administered a single SC dose of 80 mg 26 TACI CRD2-Fc as described above. 26 TACI CRD2-Fc was well tolerated, with no serious or severe TEAEs, IgG<3 g/L, or administration-related reactions.

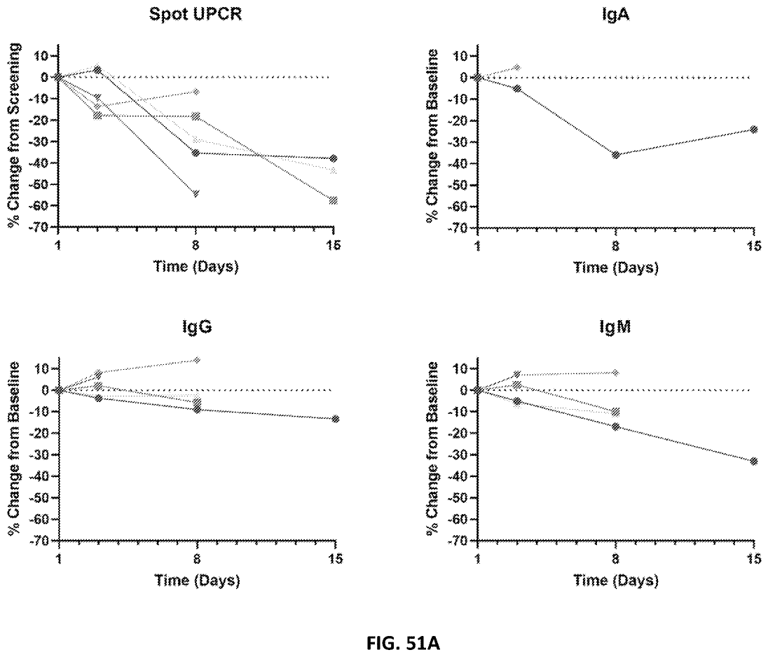

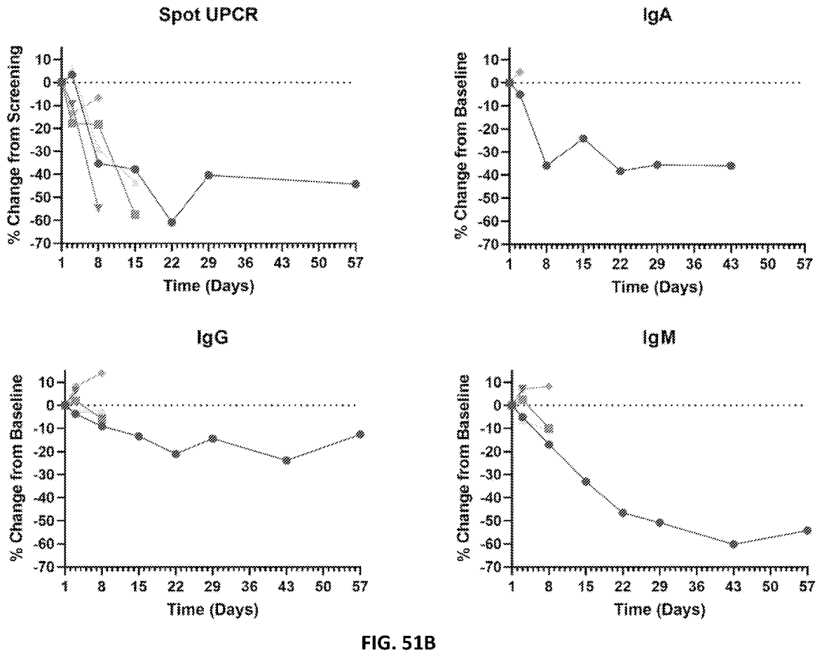

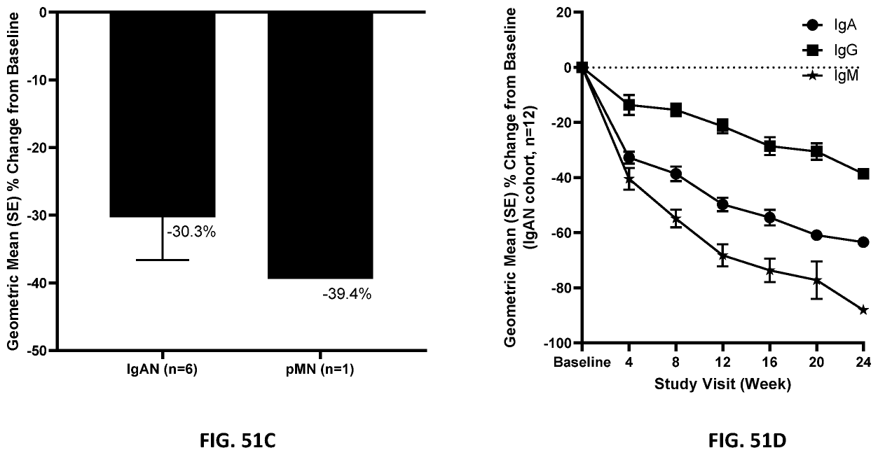

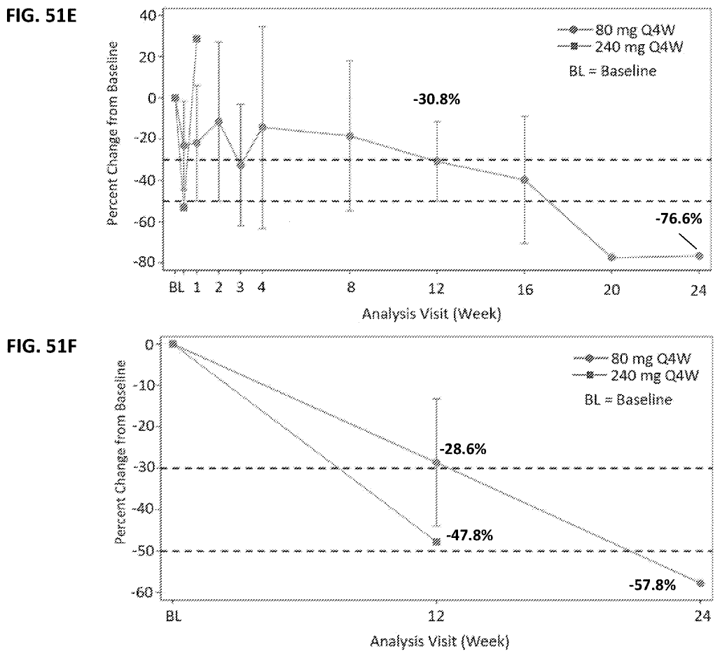

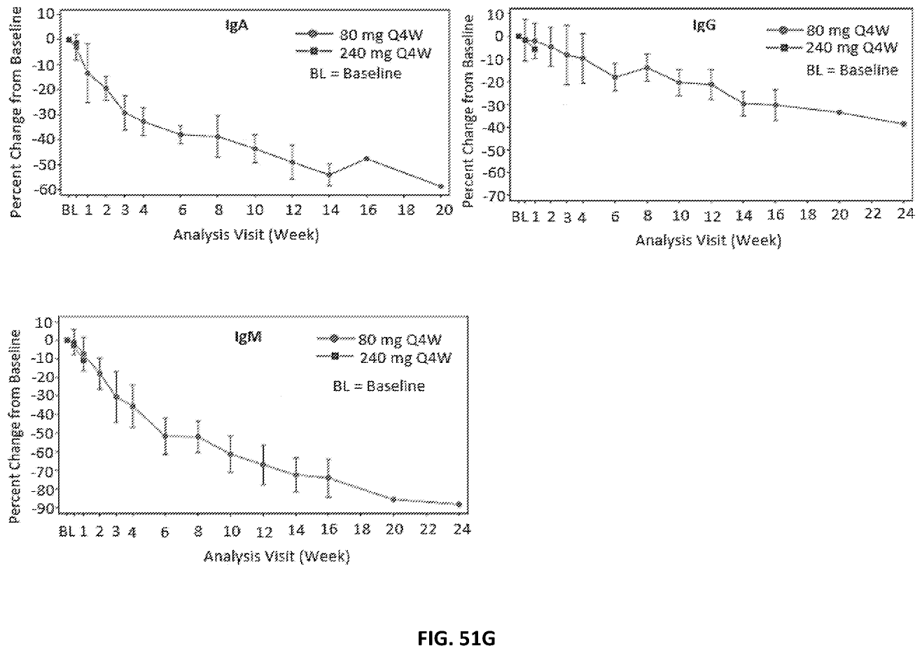

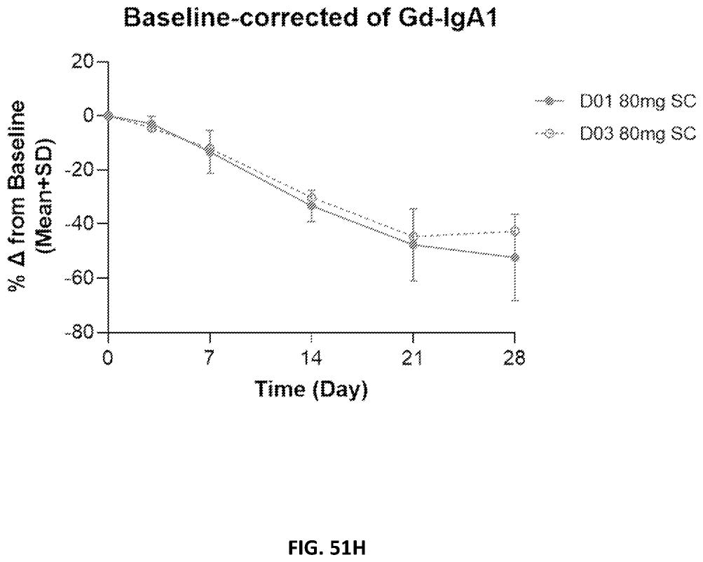

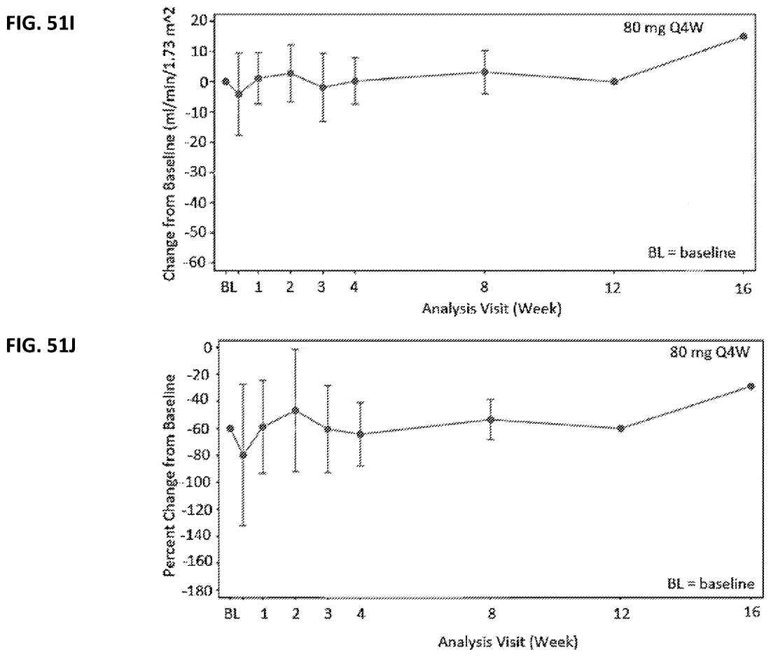

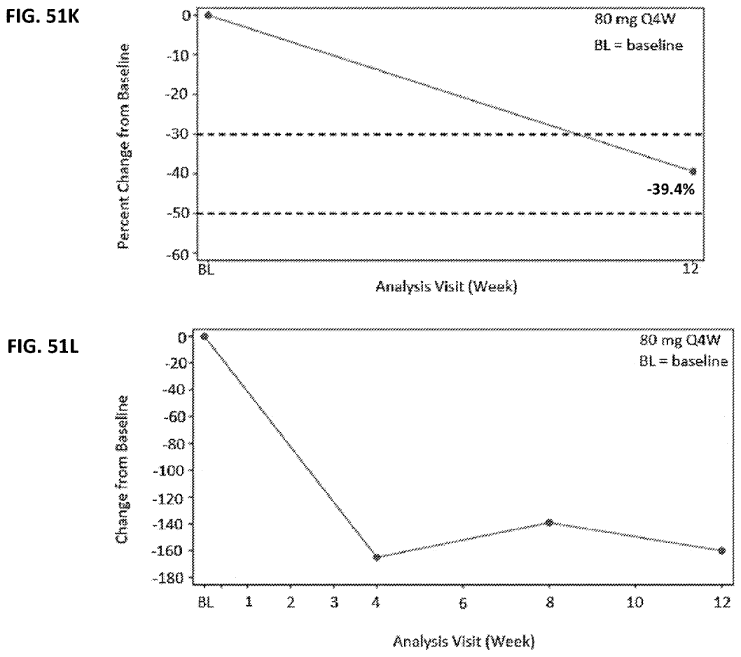

IgAN patients (n=5) that were administered a single dose of 80 mg TACI CRD2-Fc by SC administration showed decreased urine protein: creatine ratio (UPCR) 8 to 15 days post 26 TACI CRD2-Fc administration in a single (“spot”) test (FIG. 51A, see also FIG. 51B). In IgAN patients, UPCR was reduced by 30% at 12 weeks post 26 TACI CRD2-Fc administration and expected decreases in Ig levels were seen (FIG. 51C and FIG. 51D, respectively). Additionally, the first enrolled IgAN patient achieved a >78% reduction in UPCR (<0.56 g/g) by 24 weeks, preceded by a 43% reduction in Gd-IgA1 at 4 weeks (data not shown). In these IgAN patients, a general trend towards reductions in Ig levels (serum IgA, IgG, IgM levels), as demonstrated by their corresponding changes from baseline over time (FIG. 51A, see also FIG. 51B).

Dose-dependent, on-target reductions in circulating immunoglobulins was shown for IgM>IgA>IgG.

A pMN patient that was administered a single dose of 80 mg TACI CRD2-Fc by SC administration had a 39% UPCR reduction (FIG. 51C).

### Example 25. Administration of TACI-Fe Fusion Protein in Subjects with Glomerulonephritis, Including IgA Nephropathy, Lupus Nephritis and Primary Membranous Nephropathy

Adults with a diagnosis of active glomerulonephritis (GN), including active IgA nephropathy, IgAN), active lupus nephritis (LN) or active primary membranous nephropathy (PMN), were administered a composition containing TACI vTD-Fc fusion protein designated 26 TACI CRD2-Fc (Fc fusion protein set forth in SEQ ID NO:167). Safety and response to administration of the TACI vTD-Fc fusion protein were assessed. The study was part of a clinical trial study.

A group of adult human subjects were selected for administration of 26 TACI CRD2-Fc fusion protein. The subjects were those with:

IgA Nephropathy (IgAN) with a biopsy-confirmed diagnosis within ≤10 years prior to the start of screening, characterized by proteinuria ≥0.5 g/g urine total protein to creatinine ratio (UPCR); and on maximal (or maximally tolerated) dose of ACEi/ARBs>12 week prior to baseline. Background SOC immunosuppressive treatment is prohibited.

Lupus Nephritis (LN) that is LN Class III-V with a biospy-confirmed diagnosis within ≤3 years prior to the start of screening and characterized by proteinuria ≥1 g/g urine total protein to creatinine ratio (UPCR); with active urinary sediment; positive anti-dsDNA and positive antinuclear antibody (ANA) (titer ≥1:80); standard of care therapy, such as stable background immunosuppression for ≥4 weeks prior to start of the screening for pure LN or Class V LN; and UPCR between 1 to 3.5 g/g. Background immunosuppressive therapy is optional.

Primary Membranous Nephropathy (pMN) with positive anti-PLA2R or anti-THSD7A with a biopsy-confirmed diagnosis or serum negative for pathogenic autoantibodies at screening and biopsy-confirmed diagnosis within ≤5 years prior to the start of screening; less than 50% reduction of proteinuria in the last 24 weeks while on maximally tolerated angiotensin-converting enzyme (ACE)/angiotensin receptor blocker (ARB) for >12 weeks; and screening proteinuria ≥3.5 g/g urine total protein to creatinine ratio (UPCR) or UPCR≥1 g/g with serum pathogenic autoantibody positive. With the exception of calcineurin inhibitors (CNIs), background immunosuppression is prohibited.

Additional inclusion criteria may include subjects who have received SGLT2 inhibitor treatment. Exclusion criteria may include: a subject that received a prior diagnosis of, or fulfills diagnostic criteria for, another glomerular disease; a subject having a secondary IgAN; a subject having a secondary MN; an eGFR<30 mL/min/1.73m2 or rapidly progressive glomerulonephritis; a subject having a recent serious or ongoing infection; and/or a subject having a risk or history of serious infection.

Subjects were administered a subcutaneous (SC) injection of 26 TACI CRD2-Fc fusion protein at 80 mg SC Q4W or 240 mg SC Q4W (if 80 mg SC Q4W was well tolerated determined by Safety Monitoring Committee). Primary treatment was for 24 weeks, with an optional 24-week extension. Extension criteria included that the subjects had no grade ≥3 Treatment Emergent Adverse Events (TEAEs), the treatment demonstrated clinical benefit (e.g. UPCR improvement >25% by Week 20 to 24), the subject met all applicable eligibility criteria, and medical monitor approval was gained. Safety, PK, PD and anti-drug antibody (ADA) were assessed. Safety endpoints included incidences of Treatment Emergent Adverse Events (TEAEs), Severe Adverse Events (SAEs), adverse events of interest, dose-limiting toxicities, and treatment-emergent clinically significant abnormalities were monitored.

PD was assessed by measurement of biomarkers, including soluble ligands (APRIL, BAFF); serum IgG, IgA and IgM; and circulating B cell subsets. Exploratory PD assessment also included change from baseline in disease-related antibodies: Gd-IgA1 and anti-Gd-IgAQ1 (IgAN), anti-DNA such as anti-dsDNA (LN), or anti-PLA2R1 or anti-THSD7A (pMN). Exploratory efficacy endpoints included change from baseline at Week 24 in UPCR and renal response at Week 24.

As of a further data cut in an ongoing study with 12 IgAN patients recruited, in a spot UPCR test there was a 30% reduction in UPCR at 12 weeks (FIG. 51E) and a 76% reduction in UPCR at 24 weeks (FIG. 51E) when IgAN patients received 80 mg 26 TACI CRD2-Fc by SC administration every 4 weeks (Q4W). In a 24 hour UPCR test, there was a 28% reduction in UPCR at 12 weeks and a 57% reduction in UPCR at 24 weeks in IgAN patients that received 80 mg 26 TACI CRD2-Fc by SC administration Q4W (FIG. 51F). There was a 47% reduction at 12 weeks in IgAN patients that received 240 mg 26 TACI CRD2-Fc by SC administration Q4W (FIG. 51F). Overall, at 12 weeks, 2 patients achieved complete remission as defined by a UPCR of <0.5 mg/mg.

Dose-dependent, on-target reductions in circulating immunoglobulins were shown for IgM>IgA>IgG (FIG. 51G). More specifically, 2 IgAN patients receiving 80 mg 26 TACI CRD2-Fc by SC administration Q4W had reduced levels of Gd-IgA1 over 28 days (FIG. 51H). Finally, IgAN patients that received 80 mg 26 TACI CRD2-Fc by SC administration Q4W experienced increases in eGFR across 16 weeks represented as a change from baseline in FIG. 51I and as a percent change from baseline in FIG. 51J.

A pMN patient that was administered 80 mg TACI CRD2-Fc by SC administration Q4W had a 77% reduction in anti-PLA2R at 12 weeks (FIG. 51L). Further, in a 24-hour UPCR test, there was a 39.4% reduction in UPCR at 12 weeks (FIG. 51K).

### Example 26. Administration of TACI-Fc Fusion Protein in Subjects with Autoimmune Cytopenia

Subjects with a diagnosis of active cytopenia are administered a composition containing TACI vTD-Fc fusion protein designated 26 TACI CRD2-Fc (Fc fusion protein set forth in SEQ ID NO:167). The subjects are subjects 16 years of age with autoimmune cytopenia. In some cases, the subjects are adult subjects. Safety and response to administration of the TACI vTD-Fc fusion protein are assessed. The study is part of an ongoing clinical trial study.

A group of human subjects (e.g. ≥16 years of age, including adult subjects) are selected for administration of 26 TACI CRD2-Fc fusion protein. The subjects are those diagnosed with (1) Immune Thrombocytopenia (ITP)≥3 months (i.e. ≥12 weeks) prior to screening and characterized by sustained low platelet counts (e.g., <30,000/μL), and having received ≥2 prior treatments (such as in some cases ≥4 prior treatments) and with response to ≥1 treatment, and sustained thromobocytopenia on 2 occasions during screening; or (2) primary Autoimmune Hemolytic Anemia (AIHA) (e.g., warm Autoimmune Hemolytic Anemia (wAIHA))≥3 months (i.e. ≥12 weeks) prior to screening, ≥2 prior treatments, characterized by sustained Hb≥9 g/dL, and sustained anemia on 2 occasions during screening, and experiencing symptoms from anemia; or (3) primary Cold Agglutinin (CAD)≥3 months (i.e. ≥12 weeks) prior to screening, ≥2 prior treatments, characterized by sustained Hb≥9 g/dL, sustained anemia on two occasions during screening, and experiencing symptoms from anemia. Additional inclusion criteria include stable immunosuppression of the disease, if applicable or if receiving standard-of-care medications, doses must be stable. Exclusion criteria include subjects that have secondary cytopenia (e.g. systemic autoimmune disease, malignancy) or Evans Syndrome. Exclusion criteria include recent serious or ongoing infection, or risk or history of serious infection.

Additional inclusion criteria are shown in Table E42A and exclusion criteria are shown in Table E42B.

Subjects are administered a subcutaneous (SC) injection of 26 TACI CRD2-Fc fusion protein at 240 mg SC Q4W. In some cases, subjects are administered a subcutaneous (SC) injection of 26 TACI CRD2-Fc fusion protein at 80 mg SC Q4W. A placebo group is administered placebo treatment Q4W. Primary treatment is for 24 weeks, with an option 24-week extension. Thus, the subjects treated include those treated for a 48-week treatment period, with the last dose on week 44 or Day 309 of treatment. A dose escalation >240 mg is considered based on safety/tolerability.

Study endpoints are monitored with the primary objective being safety and tolerability. In some embodiments, a study endpoint is durable response (DR). Incidences of Treatment Emergent Adverse Events (TEAEs), Severe Adverse Events (SAEs), and adverse events of interest, dose-limiting toxicities, and treatment-emergent clinically significant abnormalities are monitored.

Disease activity is determined by incidence of response, durable response, time to response, use of rescue therapy, and/or hemolysis. Change in disease-related antibodies are monitored, including anti-platelet and anti-RBC antibodies. In some embodiments, disease-related antibodies are monitored by a Coombs test, a direct antiglobulin test (DAT), and/or an indirect antiglobulin test (IAT). Safety, PK and anti-drug antibody (ADA) also are assessed. PD is assessed by measurement of biomarkers, including soluble ligands (APRIL, BAFF); serum IgG, IgA and IgM; and circulating B cell subsets. Additional tested biomarkers include GPIIb/IIIa, GPIb-IX, Rh, and glycophorins. Changes in platelet response are monitored in subjects with ITP. Changes in hemoglobin response are monitored in subjects with wAIHA and CAD.

TABLE E42A
Inclusion Criteria for ITP, wAIHA and CAD

| | ITP | wAIHA | CAD |
|---|---|---|---|
| History | Primary ITP >3 months | Primary wAIHA | Diagnosis of |
| | Platelets <30,000/μL | documented with | primary CAD >3 |
| | prior to screening | a positive DAT | months based on all |
| | | for anti-IgG | of the following |
| | | (±C3d) >3 months | criteria: |
| | | Hemoglobin | Chronic hemolysis |
| | | (Hb) ≤9 g/dL | Polyspecific DAT |
| | | prior to screening | positive |
| | | | Monospecific DAT |
| | | | strongly positive |
| | | | for C3d |
| | | | Cold agglutinin |
| | | | titer ≥64 at 4° C. |
| | | | IgG DAT ≤1+ |
| | | | No overt malignant |
| | | | disease |
| | | | Hb ≤9 g/dL prior to |
| | | | screening |
| Other | N/A | At least one of the | At least one of the |
| Labs at | | following: | following: |
| screening | | Haptoglobin < LLN | Haptoglobin < LLN |
| | | Indirect | Indirect |
| | | bilirubin > ULN | bilirubin > ULN |
| | | LDH > ULN | LDH > ULN. |
| | | (+) IgG DAT | (+) IgG DAT CAD |
| | | wAIHA | Hb <10 g/dL |
| | | Hb <10 g/dL | Evidence of |
| | | Evidence of | hemolysis by |
| | | hemolysis by | haptoglobin, |
| | | haptoglobin, | iBili or LDH |
| | | iBili or LDH | |
| Prior | A history of failure | A history of failure | A history of failure |
| Treatments | or relapse to at | or relapse to at | or relapse to at |
| | least 2 treatment | least 2 treatment | least 2 treatment |
| | regimens for ITP. | regimens for wAIHA. | regimens for CAD |
| | A history of exposure | | |
| | to a TPO-RA, | | |
| | unless otherwise | | |
| | contraindicated | | |
| | or unavailable. | | |
| | ≥1 TPO-RA and ≥1 | | |
| | prior response | | |
| | No concomitant | | |
| | immunosuppression | | |
| | except corticosteroids | | |
| | (≤20 mg) | | |

Age ≥18 years
Intent to maintain any background therapy thru treatment period
(e.g., corticosteroids ≤20 mg, TPO-RA, ESA, or oral immunosuppressants)

TABLE E42B
Exclusion Criteria for ITP, wAIHA and CAD

| | | | |
|---|---|---|---|
| Criteria | ITP | wAIHA | CAD |

| | |
|---|---|
| Secondary causes | Systemic autoimmune disease, infection, or malignancy. |
| Prohibited concomitant | Rituximab, other agents that directly deplete B lymphocytes: 12 |
| medications at | weeks. Subjects who have received such therapies within 24 weeks |
| screening, unless | prior to screening must have B cell counts and serum |
| currently taking and | immunoglobulins (IgG, IgM, IgA) within reference ranges by the |
| will be maintained | time of screening. |
| during the study | IVIG, alemtuzumab, interferon alpha, interferon beta, belatacept, |
| | eculizumab, adalimumab, infliximab, certolizumab, etanercept, |
| | golimumab, sutimlimab, other marketed biological therapeutics: 8 |
| | weeks. |
| | Plasmapheresis, plasma exchange, or double-filtration |
| | plasmapheresis: 8 weeks. |
| | Transfusions with blood, blood products or other rescue |
| | medications: 2 weeks |
| | Splenectomy: 3 months |
| Prohibited conditions | Secondary autoimmune cytopenia including Evans |
| | Prior B cell depletion or BAFF/APRIL inhibitors ≤24 wk (12 wk |
| | ok for BCD if normal B cells and Ig levels) |
| | Known immunodeficiency, including IgG <700 or IgA <10 mg/dL, |
| | History of demyelinating disorder |
| | B cells <100/mL, or CD4 <200/mm3 |

### Example 27. Administration of TACI-Fc Fusion Protein in Subjects with Autoimmune Cytopenia

Subjects with a diagnosis of active cytopenia were administered a composition containing TACI vTD-Fc fusion protein designated 26 TACI CRD2-Fc (Fc fusion protein set forth in SEQ ID NO:167). The subjects were adult subjects (≥18 years of age) with autoimmune cytopenia. Safety and response to administration of the TACI vTD-Fc fusion protein were assessed. The study is part of an ongoing clinical trial study.

A group of human adult subjects wee selected for administration of 26 TACI CRD2-Fc fusion protein. The subjects were those diagnosed with (1) primary Immune Thrombocytopenia (ITP)≥3 months (i.e. ≥12 weeks) prior to screening and characterized by sustained low platelet counts (e.g., <30,000/μL), and having a history of failure or relapse to 2 prior treatments for ITP including but not limited to steroids, thrombopoietin receptor agonists (TPO-RAs) and a history of response to ≥1 prior treatment, and sustained thromobocytopenia on 2 occasions during screening; or (2) primary Autoimmune Hemolytic Anemia (AIHA) (e.g., warm Autoimmune Hemolytic Anemia (wAIHA))≥3 months (i.e. ≥12 weeks) with a current or prior positive antiglobulin test (DAT) or anti-IgG prior to screening, documented history of Hb≤9 g/dL, and sustained symptomatic anemia on 2 occasions during screening; a history of failure or replase to at least 2 treatments for wAIHA or (3) primary Cold Agglutinin (CAD)≥3 months (i.e. ≥12 weeks) prior to screening, documented history of Hb≤9 g/dL, sustained symptomatic anemia on two occasions during screening, and a history of failure or relapse to at least 2 treatments for CAD. Additional inclusion criteria include stable background therapy, if applicable or if receiving standard-of-care medications. Exclusion criteria include subjects that have secondary cytopenia (e.g. systemic autoimmune disease, malignancy) or Evans Syndrome; recent serious or ongoing infection, or risk or history of serious infection.

Subjects were administered a subcutaneous (SC) injection of 26 TACI CRD2-Fc fusion protein at 240 mg SC Q4W. Primary treatment was for 24 weeks, with an optional 24-week extension. Study endpoints were monitored with the primary objective being safety and tolerability. Safety endpoints included incidences of Treatment Emergent Adverse Events (TEAEs), Severe Adverse Events (SAEs), and adverse events of interest, dose-limiting toxicities, and treatment-emergent clinically significant abnormalities were monitored.

PK and anti-drug antibody (ADA) were also assessed. PD was assessed by measurement of biomarkers, including soluble ligands (APRIL, BAFF); serum IgG, IgA and IgM; and circulating B cell subsets. Additional exploratory PD endpoints included change from baseline in disease-related antibodies such as anti-platelet antibodies. Exploratory efficacy endpoints included incidence of response (platelet response for ITP; Hb response for wAIHA and CAD), duration of response, time to response, use of rescue therapy, and/or hemolysis.

### Example 28. Administration of TACI-Fc Fusion Protein in Subjects with Bullous Dermatosis

Adults with a diagnosis of active autoimmune blistering disease (ABD) are administered a composition containing TACI vTD-Fc fusion protein designated 26 TACI CRD2-Fc (Fc fusion protein set forth in SEQ ID NO:167). Safety and response to administration of the TACI vTD-Fc fusion protein are assessed. The study is part of an ongoing clinical trial study.

A group of adult human subjects are selected for administration of 26 TACI CRD2-Fc fusion protein. The subjects are those diagnosed with (1) Pemphigus vulgaris or foliaceus characterized by Pemphigus disease area index (PDAI)≥15 with positive anti-Dsg1 or anti-Dsg3; or (2) Pemphigoid characterized by Investigator's Global Assessment (IGA) TBD with positive anti-BP180 or anti-BP230. Additional inclusion criteria include stable immunosuppression of the disease, if applicable. Exclusion criteria include subjects that have secondary disease (e.g. paraneoplastic).

Subjects are administered a subcutaneous (SC) injection of 26 TACI CRD2-Fc fusion protein at 80 mg SC Q4W or 240 mg SC Q4W. In some cases, a 2-stage Fleming Design is used. Patients are monitored, including by photo documentation.

Incidences of Treatment Emergent Adverse Events (TEAEs), Severe Adverse Events (SAEs), and adverse events of interest, dose-limiting toxicities, and treatment-emergent clinically significant abnormalities are monitored.

Study endpoints include disease activity as determined by complete remission or treatment success, PDAI or Bullous pemphigoid disease area index (BPDAI), and/or corticosteroid usage. Change in disease-related antibodies are monitored, including anti-dsg1/3 or anti-BP180/230. Safety, PK and anti-drug antibody (ADA) also are assessed. PD is assessed by measurement of biomarkers, including soluble ligands (APRIL, BAFF); serum IgG, IgA and IgM; and circulating B cell subsets.

### Example 29. Administration of TACI-Fc Fusion Protein in a Mouse Model of AIHA

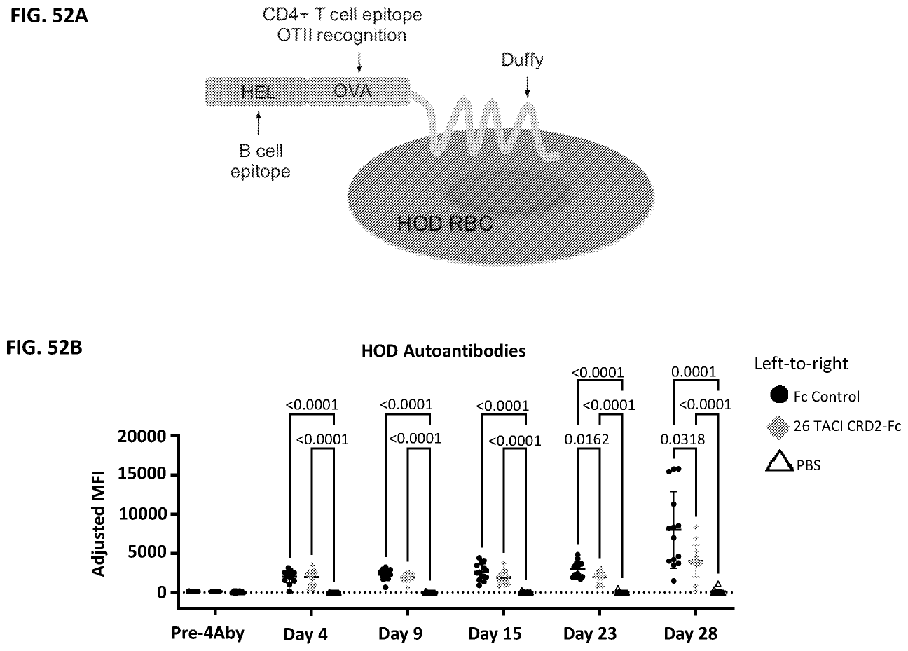

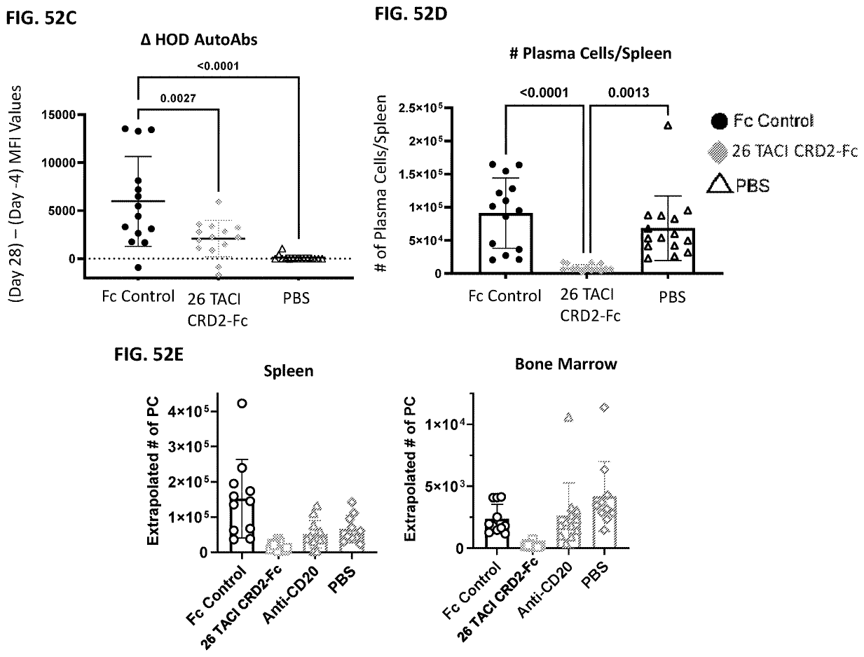

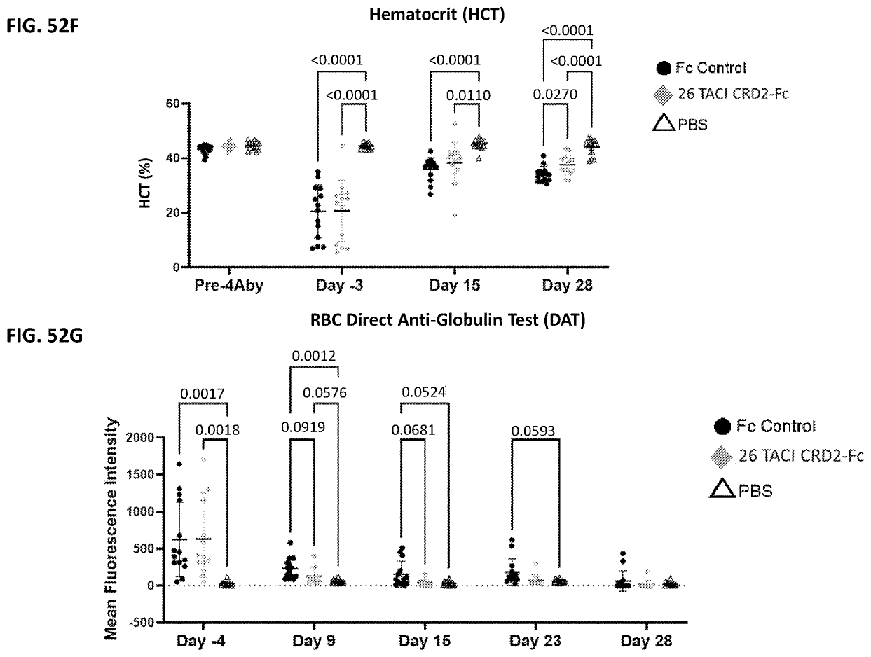

The TACI-Fc fusion protein, 26 TACI CRD2-Fc, was investigated in a mouse model of autoimmune anemia (AIHA) involving HEL-OVA-Duffy (HOD) mice cross-bred with OTII transgenic mice (HOD×OTII), which represent a model system that reflects the pathogenesis observed in human patients (Desmarets et al., Blood, 2009; 114(11):2315-2322). HOD mice express an RBC-restricted triple fusion protein of hen egg lysozyme (HEL), a portion of ovalbumin (OVA), and human blood group molecule Duffy (FIG. 52A). OTII mice express a transgenic T cell receptor specific for OVA presented in the context of the MHC class II molecule I-Ab. AIHA develops in aged HOD×OTII mice, or alternatively the disease onset can be accelerated with administration of monoclonal antibodies (mAb) against checkpoint inhibitors and IL-10R to reverse tolerance. Disease in this model is accompanied by high titer RBC autoantibodies, increased activated B cells, decreased hematocrit, reticulocytosis, and splenomegaly.

In order to study the role of 26 TACI CRD2-Fc in the HOD mouse model of AIHA, young HOD×OTII mice (1.5 to 3.5 months old) were intraperitoneally (IP) administered antibodies against CTLA-4, IL-10R, LAG-3, and PD-1 for 2.5 weeks to accelerate disease onset, then randomized to treatment groups (N=15) based on HOD autoantibody titers and treated IP with 10 mg/kg 26 TACI CRD2-Fc or a molar-matched dose of Fc control twice weekly for 3.5 weeks. A PBS control group that did not receive the 4-antibody induction regimen was also included for comparison. Assessments included measurement of circulating RBC autoantibodies and autoantigens, hematocrit and reticulocytes over time, and immunophenotyping of B and T cell subsets in spleen and bone marrow (BM) at the end of the study (Day 29). Detection of autoantibodies bound to peripheral RBCs was evaluated by staining RBCs with goat anti-mouse Ig and measuring signal by flow cytometry. Statistical significance was determined using 1-way (FIG. 52D) or 2-way (FIG. 52B, FIG. 52C, FIG. 52E, FIG. 52F and FIG. 52G) repeated measures ANOVA w/Tukey's multiple comparisons test; p values <0.05 were considered statistically significant.

As compared to Fc control, 26 TACI CRD2-Fc treatment significantly suppressed the generation of antigen (HOD)-specific autoantibodies in serum (FIG. 52B and FIG. 52C), reduced plasma cells in spleen (FIG. 52D, FIG. 52E) and BM (FIG. 52E), significantly increased whole blood hematocrit (FIG. 52F), and tended to lower anti-erythrocyte autoantibodies (FIG. 52G).

These results establish that dosing of 26 TACI CRD2-Fc in this mouse model of AIHA resulted in significantly lower levels of plasma cells and antigen-specific autoantibodies in serum, a significant increase in hematocrit, and trends for lower anti-erythrocyte autoantibodies.

### Example 30. Administration of TACI-Fc Fusion Protein in a Murine Experimental Autoimmune Myasthenia Gravis Model (EAMG)

The variant TACI-Fc fusion protein, 26 TACI CRD2-Fc, was investigated in a mouse model of experimental autoimmune myasthenia gravis (EAMG).

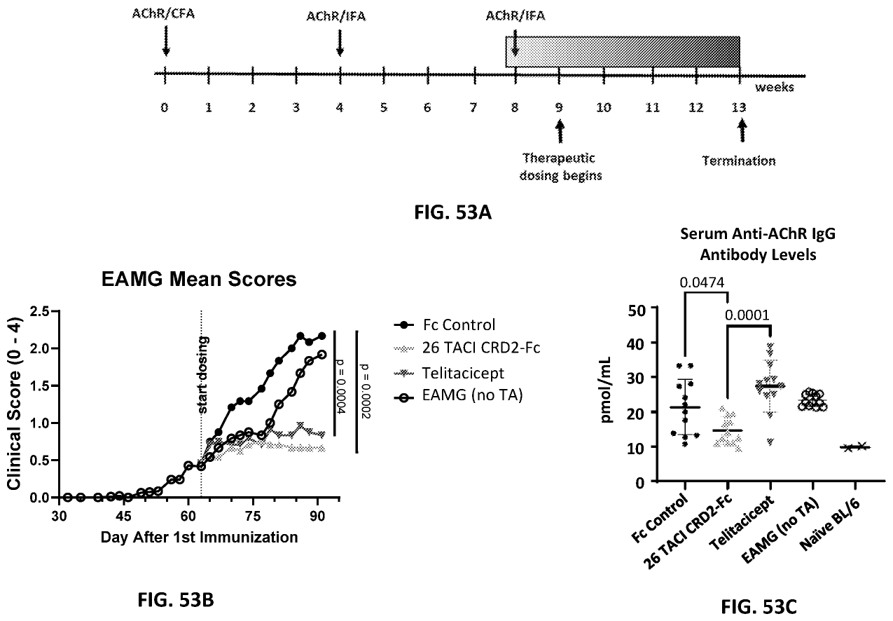

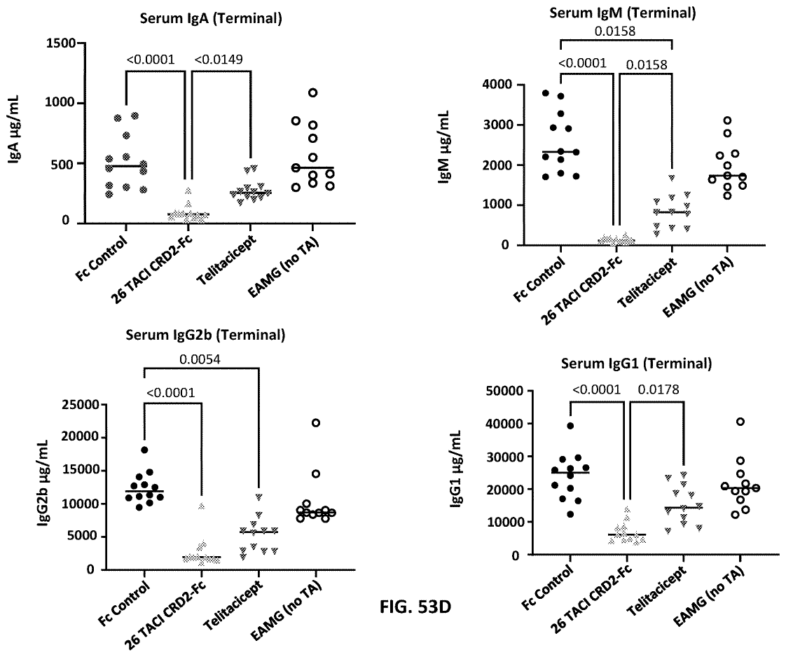

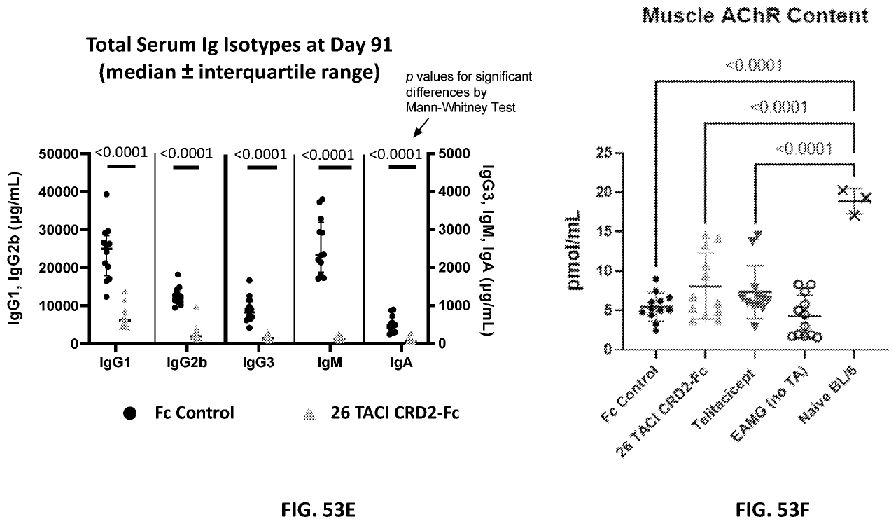

Mice were immunized with acetylcholine receptor (AChR) and later treated after disease onset with 200 μg 26 TACI CRD2-Fc or molar-matched doses of Fc control or WT TACI-Fc (telitacicept), twice weekly for 7 total injections. Specifically, EAMG was induced in female C57BL/6 mice with subcutaneous immunizations of either 20 μg of acetylcholine receptor (AchR; purified from Torpedo californica electric ray organ) in complete Freund's adjuvant (CFA) (Day 0) or incomplete Freund's adjuvant (IFA) (Day 30 and 60) (FIG. 53A). The shaded bar indicates onset and progression of disease, as defined by clinical scores ≥0.5. Some mice began showing signs of disease (score ≥0.5) as early as Day 49. Group randomization began at Day 63 to ensure that mice in each group (n=12) had similar body weights and disease scores (mean±SD of 0.46±0.33 for Fc control, 0.46±0.26 for 26 TACI CRD2-Fc, and 0.46±0.26 for telitacicept treatment groups) at the start of dosing. A group of mice (n=12) with EAMG but not treated with any of the test articles (26 TACI CRD2-Fc, Fc control or telitacicept) was included to equalize treatment groups, as needed, at the time of staging. 26 TACI CRD2-Fc and other test article dosing by intraperitoneal injection began on Day 63 and ended 7 days prior to the end of the study (i.e. Day 84).

Mice were weighed and clinical scores assigned 1-2 times per week until Day 35, and then every 2-3 days until the end of study (Day 91/Week 13) by investigators blinded to the treatment groups. Clinical score was assessed after exercise for 30 seconds, using the grip strength test. Disease severity was graded as follows: grade 0, normal strength and no abnormalities; grade 1, mildly decreased activity and weak grip; grade 2, clinical signs present before exercise (tremor, head down, hunched posture, weak grip); grade 3, severe clinical signs at rest, no grip, moribund; grade 4, terminate. Whole blood was collected weekly just prior to test article administration, beginning on the day of treatment start (Day 63), and at the end of the study. Serum was collected after centrifugation and at the end of the study. Muscles were collected after termination of the study for measurement of AChR content.

26 TACI CRD2-Fc-treated EAMG mice demonstrated significantly lower clinical disease scores over time and at termination compared to control mice (FIG. 53B). Serum anti-AChR IgG antibody levels were significantly lower (p<0.05) in the 26 TACI CRD2-Fc group vs. both the Fc control and telitacicept groups (FIG. 53C). Serum concentrations of total IgA, IgM, IgG1, and IgG2b collected at termination (Day 91) were significantly lower in the 26 TACI CRD2-Fc-treated mice as compared to Fc control and telitacicept treatment groups (FIG. 53D). Muscle AChR content (pmol/mL) was significantly lower (p<0.0001) in Fc control-, 26 TACI CRD2-Fc-, and telitacicept-treated mice compared to control mice that had not received AChR injection (FIG. 53E).

The results show that 26 TACI CRD2-Fc demonstrates promising efficacy in a preclinical EAMG model.

### Example 31. Clinical Pharmacokinetics/Pharmacodynamics (PK/PD) Modeling and Simulation of 26 TACI CRD2-Fc

The therapeutic index of 26 TACI CRD2-Fc using pharmacokinetics (PK) and pharmacodynamics (PD) modeling/simulation of repeat dosing was assessed.

Simulations were modeled to predict PK/PD models for APRIL target coverage and decreases in IgA, IgG and IgM at Q4W, Q8W, and Q12W repeat-dosing. The modeling data was based on 50 subjects.

PK/PD models were fit to exposure and PD data for a single ascending dose study in healthy volunteers. Monte Carlo simulations of these models were used to predict the potential safety risk and to estimate the potential efficacy based on a surrogate PD endpoint. Decreases in serum immunoglobulin G (IgG) were used to estimate the risk of hypogammaglobulinemia from 26 TACI CRD2-Fc subcutaneous administration in repeat-dose studies. Decreases in serum galactose-deficient immunoglobulin A1 (gd-IgAQ1) were used to predict the potential efficacy of 26 TACI CRD2-Fc in autoimmune disease exemplified within IgAN. Gd-IgA1 is the antigen of autoantibodies found in IgAN patients and is a potential biomarker of urinary protein to creatinine ratios (uPCR), which is a typical clinical endpoint in IgAN patients. These markers of safety risk and potential efficacy were used to evaluate the therapeutic index of multiple dose regimens.

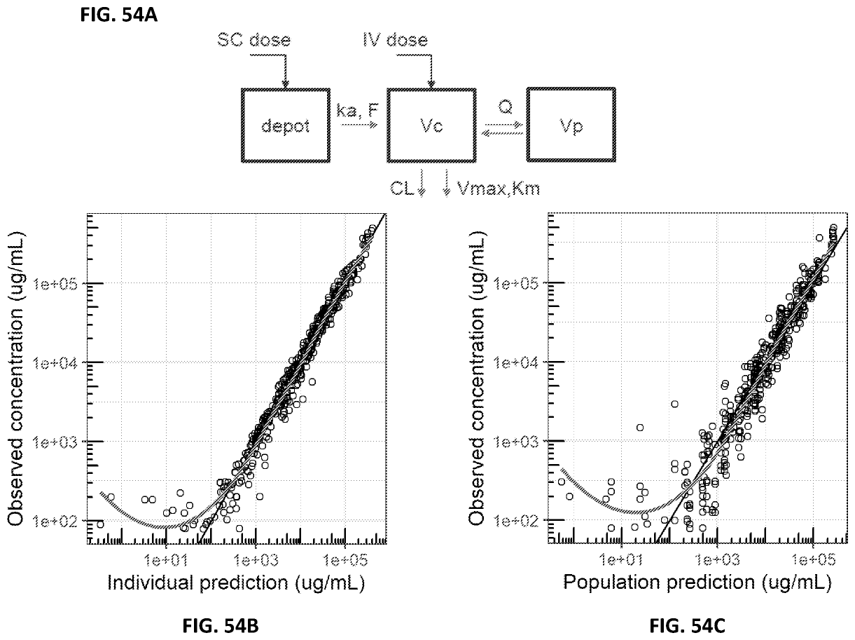

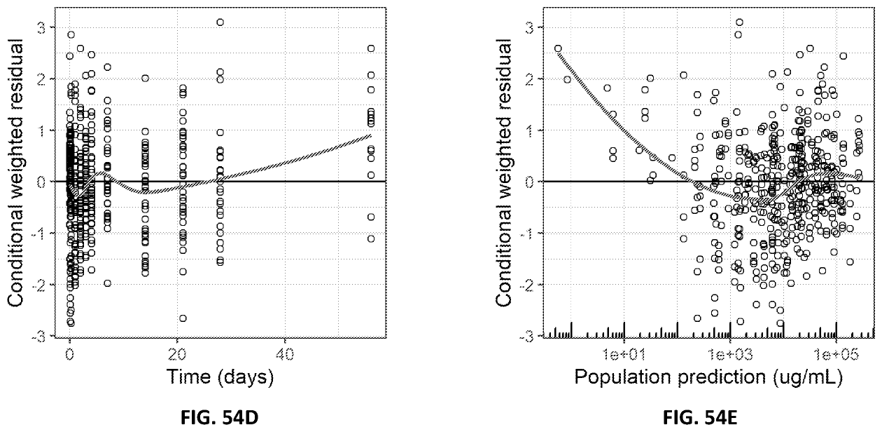

FIG. 54A depicts PK model structure for 26 TACI CRD2-Fc concentrations, comprising a two-compartment model with parallel linear and non-linear (Michaelis-Menten) clearance, and inter-individual variability estimated on CL and Vc. FIGS. 54B-54E show diagnostic plots for population pharmacokinetic analysis.

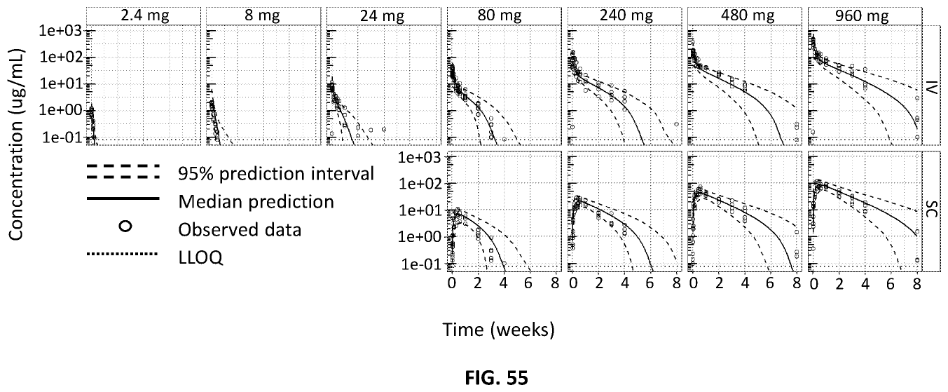

Subcutaneous doses of 26 TACI CRD2-Fc included 80 mg, 240 mg, 480 mg or 960 mg whereas intravenous doses of 26 TACI CRD2-Fc included 2.4 mg, 8 mg, 24 mg, 80 mg, 240 mg, 480 mg or 960 mg. FIG. 55 shows PK visual predictive assessment. All parameters were estimated with relative standard error (RSE)<40%. There was <20% shrinkage in the PK model.

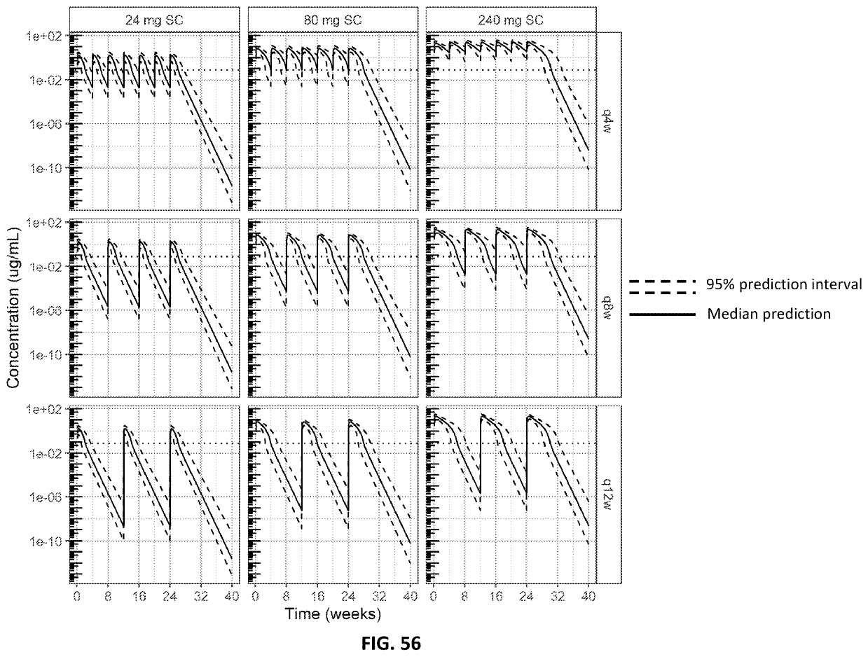

FIG. 56 shows the PK predictive assessments of 26 TACI CRD2-Fc concentration following intravenous (IV) administration at doses 2.4 mg, 8 mg, 24 mg, 80 mg, 240 mg, 480 mg or 960 mg and subcutaneous (SC) administration at 80 mg, 240 mg, 480 mg or 960 mg over 8 weeks. Based on non-compartmental analysis (NCA), the half-life of 26 TACI CRD2-Fc when administered subcutaneously at 80 mg and 240 mg is 3.7 days and 7.4 days, respectively. Repeat dosing of 26 TACI CRD2-Fc at dose intervals of 4 weeks or more was predicted to have little to no 26 TACI CRD2-Fc accumulation. With the exception of the 80 mg and 240 mg Q4W dosing regimens, non-linear clearance dominates all dose regimens simulated (FIG. 56). Additionally, due to the rapid clearance of 26 TACI CRD2-Fc, steady state is rapidly reached in 24 mg (q4w, q8w, q12w), 80 mg (q8w and q12w), and 240 mg (q8w and q12w).

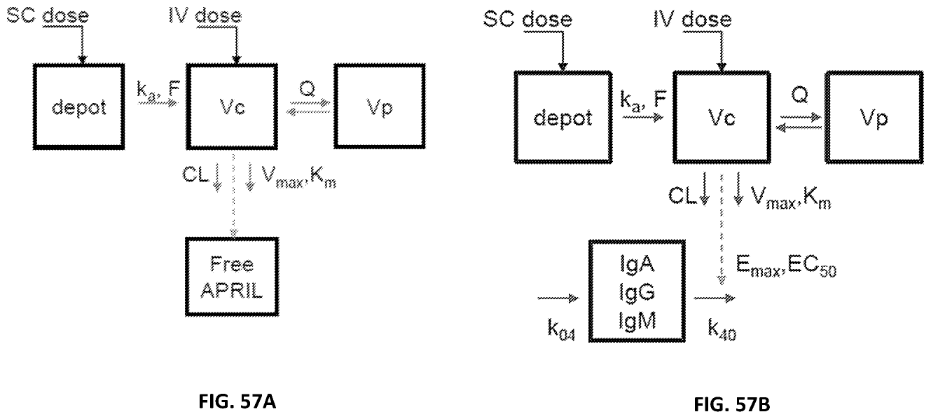

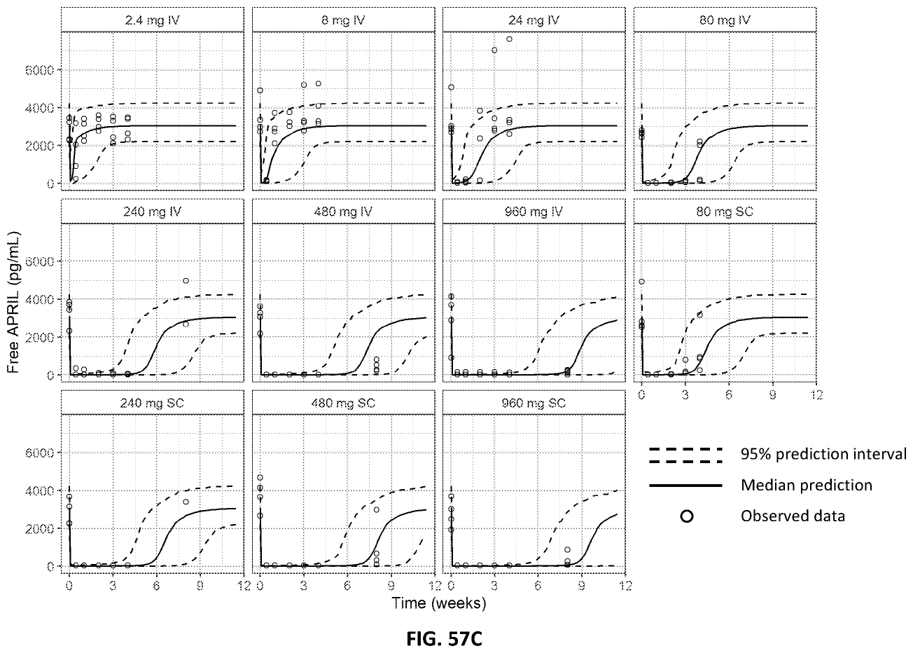

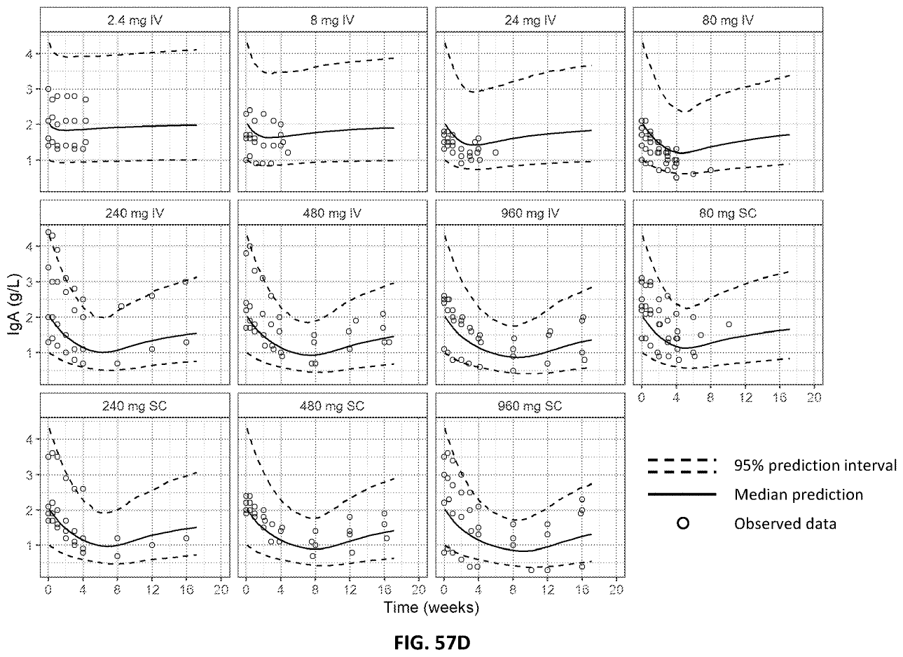

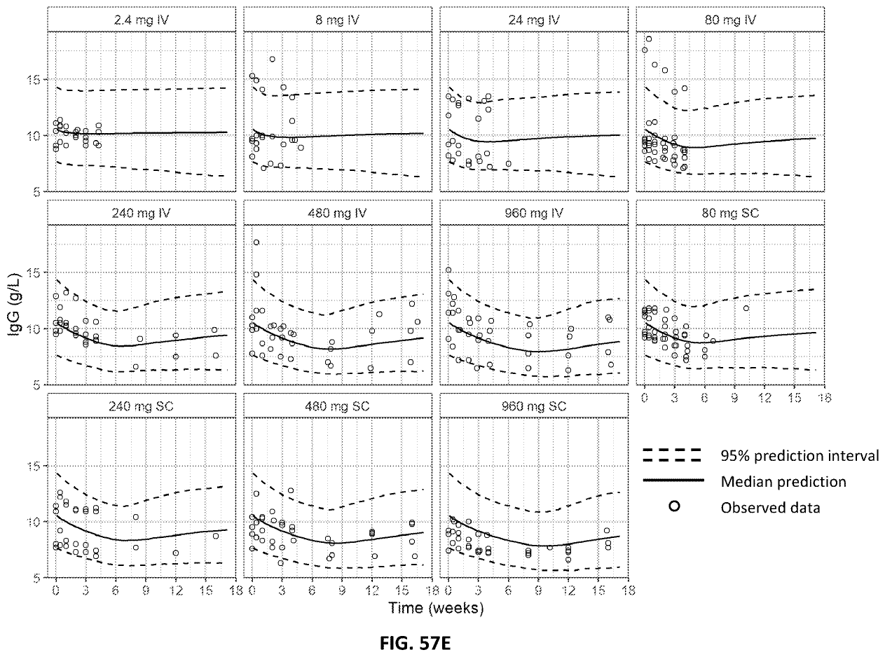

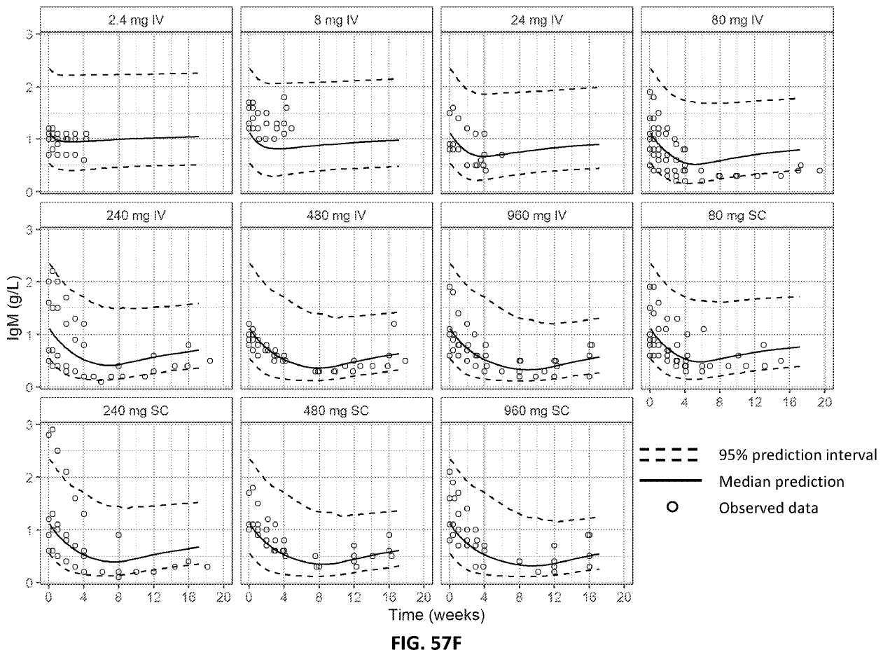

FIGS. 57A-57B show PK/PD model structures for free APRIL and immunoglobulins (IgA, IgG or IgM). APRIL, IgA, IgG and IgM PK/PD models were fitted to data. Free APRIL concentrations were best described with a direct model (FIG. 57C). IgA, IgG and IgM concentrations (g/L) were best described by an indirect model with a saturable effect (FIGS. 57D-57F, respectively).

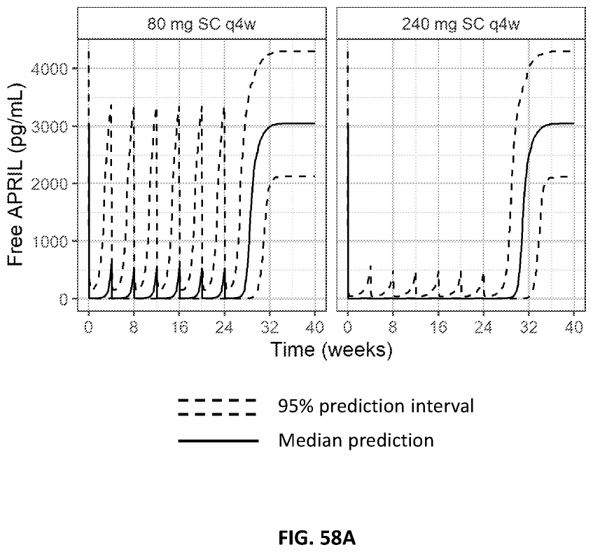

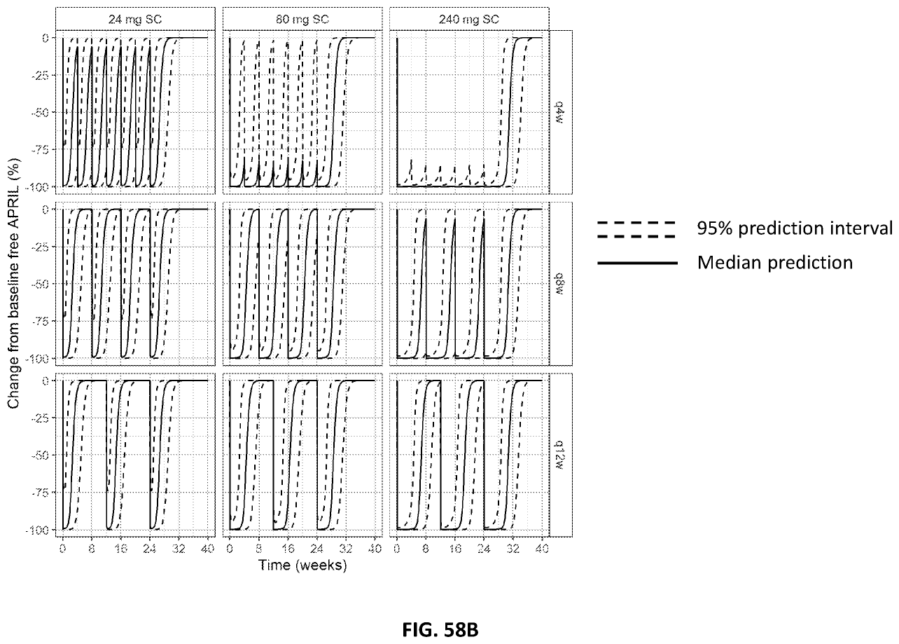

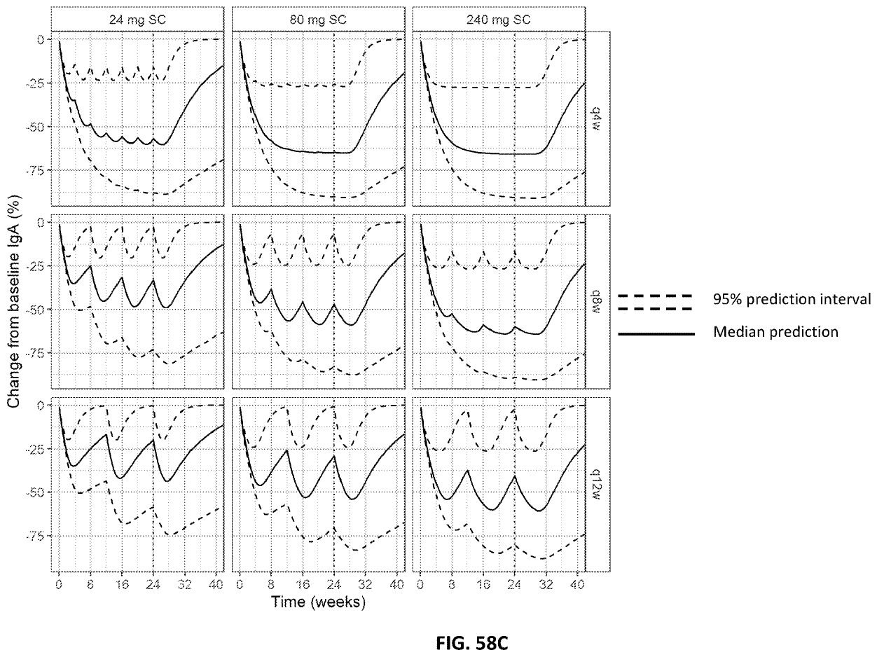

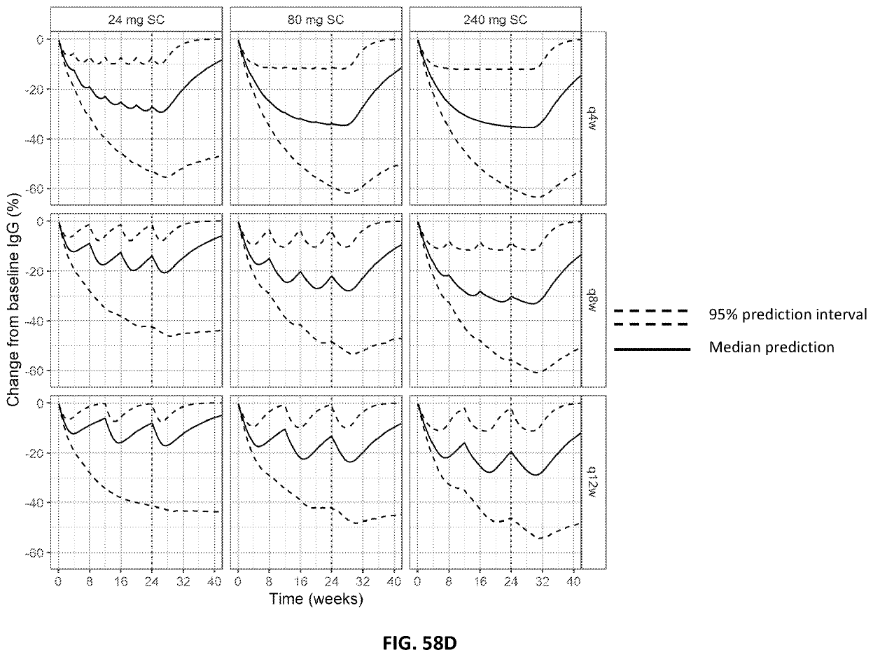

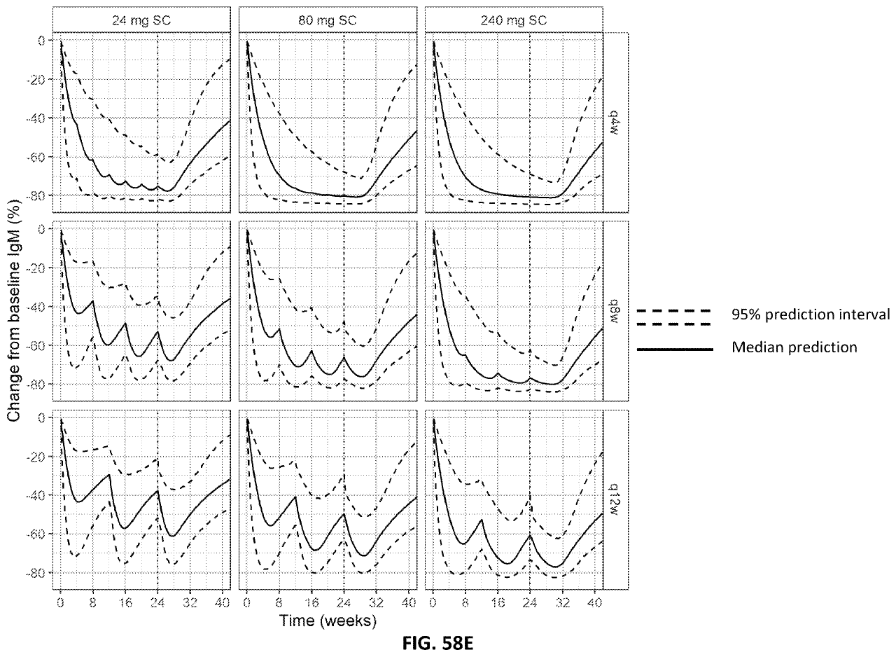

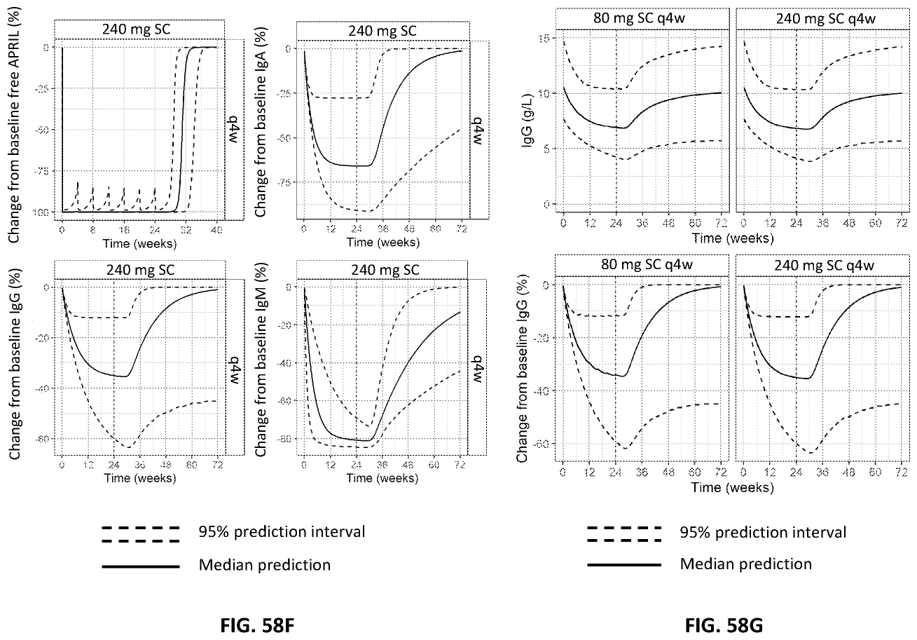

FIGS. 58A-58E depict PD simulations for 26 TACI CRD2-Fc at q4w, q8w or q12w dose regimens of 24 mg, 80 mg or 240 mg given SC up to 40 weeks for free April (FIG. 58A or FIG. 58B), IgA (FIG. 58C), IgG (FIG. 58D) or IgM (FIG. 58E). FIG. 58F provides results for the PD simulations of a Q4W dose regimen of 240 mg given SC up to 72 weeks. Simulations showed that APRIL was quickly bound by all dose regimens within <1 day and for dose regimens of 80 mg and 240 mg Q4W, median APRIL recovered to baseline levels within 8 to 10 weeks, respectively. APRIL simulations predicted that the median patient dosed 80 mg and 240 mg SC Q4W would have 75% and >100% reductions in APRIL for the entire dosing period, respectively. IgA, IgG and IgM simulations showed the following dose regimens have saturated reductions of IgA, IgG, and IgM: 80 mg SC Q4W, 240 mg SC Q4W, and 240 SC Q8W. Median IgA, IgG, and IgM reached nadir by 8-12 weeks after repeat dosing of 26 TACI CRD2-Fc. FIG. 58G provides results for the PD simulations of a Q4W dose regimen of 80 mg or 240 mg given SC up to 72 weeks. IgG simulations of 80 and 240 mg SC Q4W dose regimens predict an estimated reduction of 35% on average (ranging from 11% to 62% [95% prediction interval]) from baseline.

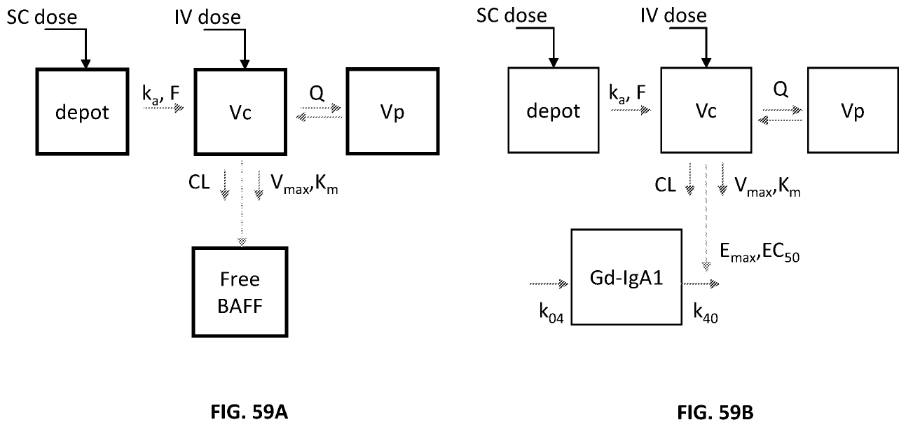

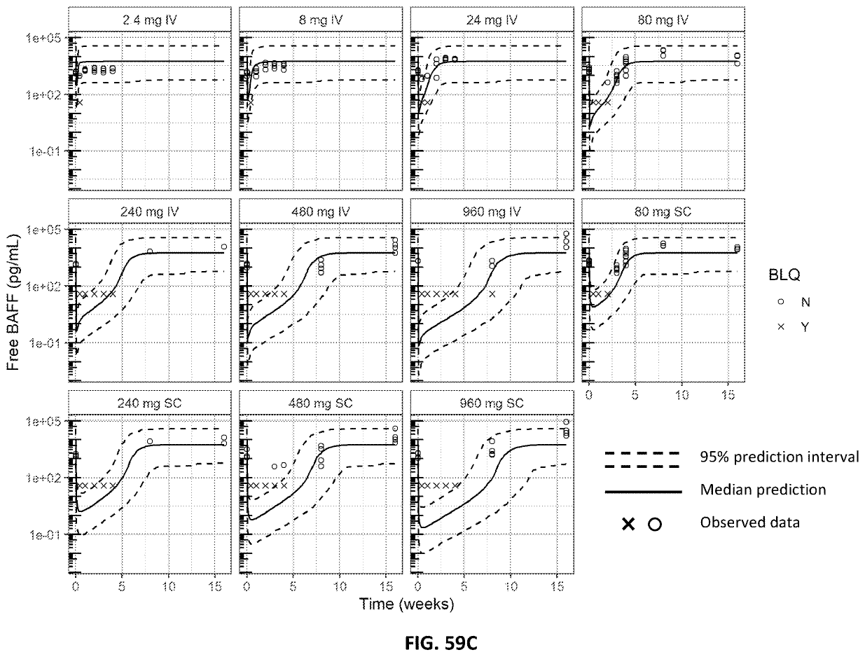

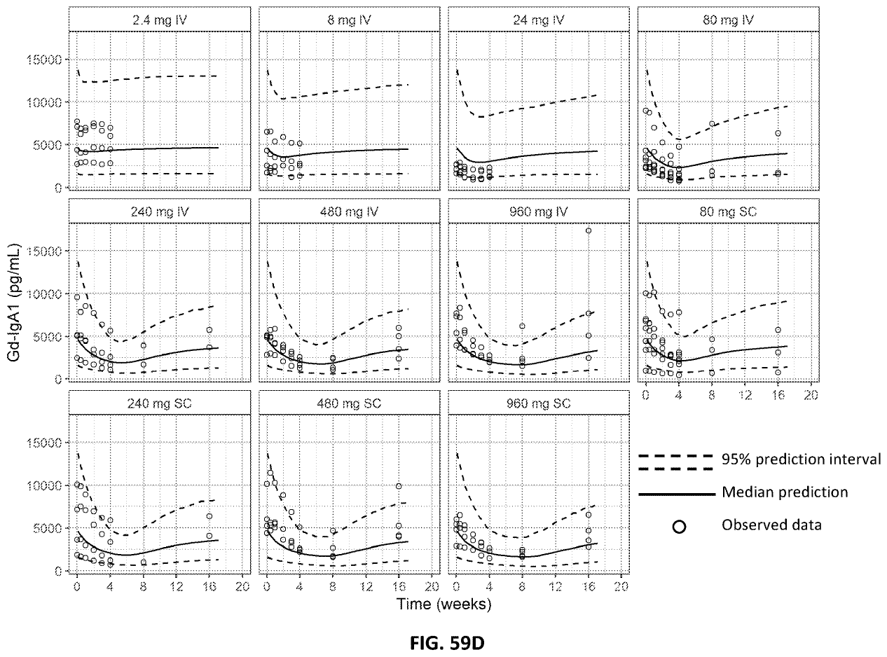

FIGS. 59A-59B show PK/PD model structures for free BAFF and Gd-IgAQ1. Free BAFF concentrations (pg/mL) were best described with a direct model (FIG. 59C). Gd-IgA1 concentrations (pg/mL) were best described by an indirect model with a saturable effect (FIG. 59D).

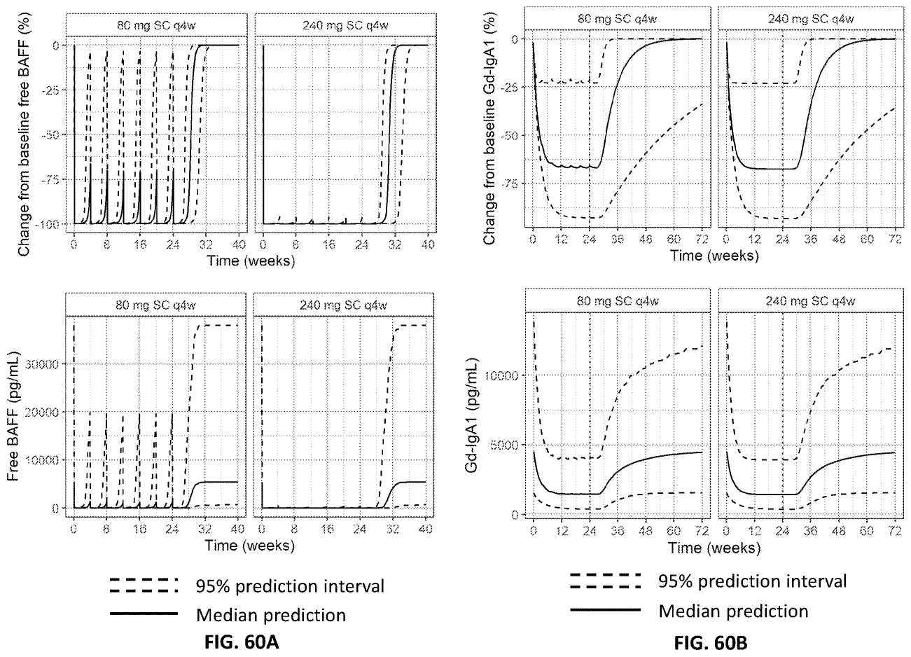

FIGS. 60A-60B depict PD simulations for 26 TACI CRD2-Fc at q4w dose regimens of 80 mg or 240 mg given SC up to 40 weeks for free BAFF (FIG. 60A) and up to 72 weeks for Gd-IgA1 (FIG. 60B). Simulations show that BAFF is quickly reduced by both dose regimens within <1 day and the median BAFF recovers to baseline levels within 8 to 10 weeks, respectively. BAFF simulations predict that the median patient dosed 80 and 240 mg SC Q4W will have 70% and >95% reductions in BAFF for entire dosing period. Gd-IgA1 simulations of 80 and 240 mg SC Q4W dose regimens estimate a mean reduction of greater than 65%. 26 TACI CRD2-Fc doses of 80 and 240 mg Q4W are similarly predicted to result in meaningful reductions in other immunoglobulins (IgA: −60% and IgM: −80%) that may further correlate with activity.

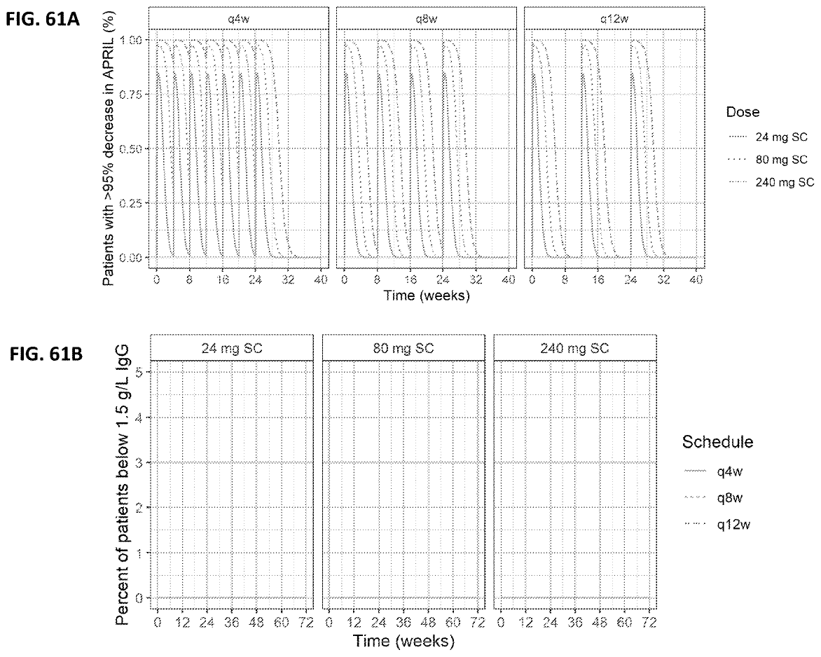

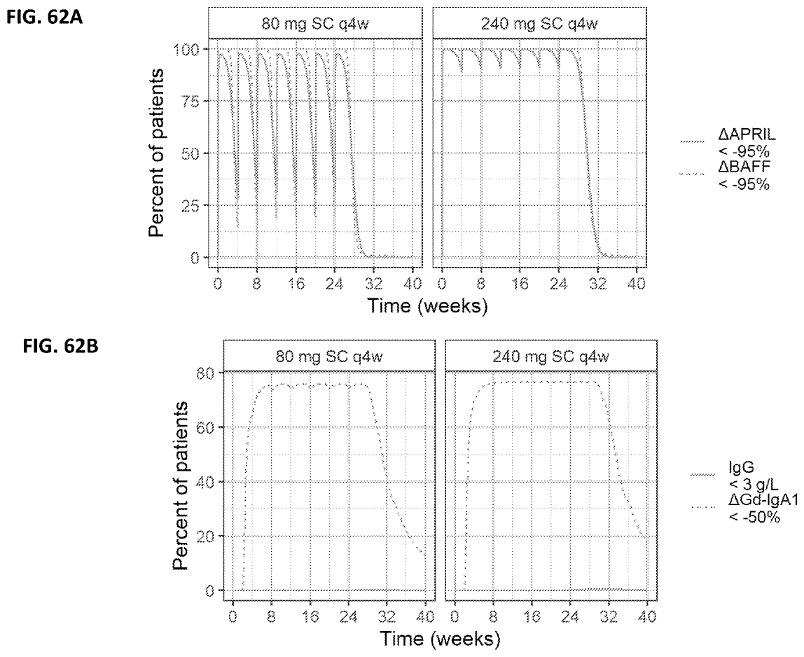

FIGS. 61A-61B depict a summary of target coverage for APRIL and decreases below 1.5 g/L for IgG. APRIL simulations showed that dose regimens of 80 mg and 240 mg SC Q4W had greater than 75% and 95% target coverage for 3 and 4 weeks at steady state, respectively (FIG. 6IA). IgG simulations predicted that dose regimens of 80 mg and 240 mg SC Q4W would result in less than 1% of patients with circulating IgG<1.5 g/L, which would result in hypogammaglobulinemia (FIG. 61B). These dose regimens are anticipated to provide adequate APRIL target coverage while minimizing risk of hypogammaglobulinemia. BAFF simulations show similar results has have been previously reported by other B-cell and BAFF inhibitors. FIGS. 62A-62B depict a summary of target coverage for APRIL/BAFF and IgG/Gd-IgAQ1, respectively. The dose regimens of 80 and 240 mg SC Q4W are predicted to have greater than 75% and 90% of patients with APRIL/BAFF target coverage for 3 and 4 weeks at steady state, respectively. Further, these dose regimens will not likely (<1%) be associated with hypogammaglobulinemia (IgG<3 g/L). Gd-IgA1 simulations predict greater than 70% of patients will have decreases in Gd-IgA1 greater than 50%.

These data indicate that 26 TACI CRD2-Fc exposure is best described by a PK model with parallel linear and nonlinear clearance; APRIL and immunoglobulin concentrations are best described by direct and indirect models with a saturable effect, respectively; and Gd-IgAQ1 concentrations are best described by indirect models with saturable effect.

IgG and Gd-IgA1 simulations show that 80 mg SC Q4W and 240 mg SC Q8W have minimally saturated reductions in these PD markers. IgG simulations of the dose regimens of 80 and 240 mg SC Q4W predict that fewer than 1% of patients will have reductions in IgG that would result in severe IgG hypogammaglobulinemia (<3 g/L IgG). These dose regimens reduce IgG by an estimated 35% from baseline. Gd-IgA1 simulations of these dose regimens estimate that greater than 75% of patients will have 50% or greater reductions in Gd-IgA1.

Dose regimens of 80 mg and 240 mg SC Q4W are anticipated to provide adequate APRIL and Gd-IgA1 target coverage and minimize the risk of hypogammaglobulinemia; and simulations of these PK/PD models support investigating the dose regimens of 80-240 mg SC every 4 weeks.

### Example 32. Assessment of the Activity of TACI-Fc Fusion and Clinically Relevant Comparators in an Accelerated (NZB×NZW)F1 (NZB/W) Mouse Model of Lupus Nephritis

This Example describes the assessment of 26 TACI CRD2-Fc and clinically relevant comparators (Table E43) to affect disease readouts in vivo in an accelerated (NZB×NZW)F1 (“NZB/W”) mouse model of lupus nephritis. NZB/W mice spontaneously develop lupus as well as signs of autoimmune hemolytic anemia (AIHA), with the majority of female NZB/W mice developing disease between approximately 22 and 43 weeks of age. The use of NZB/W mice to evaluate treatments for lupus is considered one of the best animal models of lupus currently available. The disease can be accelerated and synchronized with intravenous administration of an interferon alpha (IFNα) adenovirus when NZB/W mice are approximately 11-12 weeks of age. This accelerated model is also a well-accepted model of lupus in the field and has been used to evaluate therapeutics in development for the treatment of lupus, and mechanisms of action (Haselmayer et al. (2019) J Immunol 202(10):2888-2906; Liu et al. (2011) Arthritis Rheum 63(1):219-229; Liu et al. (2013) Front Immunol 4:306; Hawtin et al. (2023) Cell Rep Med 4(5):101036).

This Example describes a study that evaluated the activity of 26 TACI CRD2-Fc, as compared to molar-matched levels of an Fc isotype control protein (Fc Control) or telitacicept, or dose levels shown to be efficacious in mice of efgartigimod, a depleting (i.e., rat IgG2b) anti-mouse CD20 monoclonal antibody, or cyclophosphamide to reduce readouts of lupus and AIHA in this IFNα accelerated NZB/W model (Julien et al. (2023) Rheumatology (Oxford) kead298; T. P. Casey (1968) 32(3):436-444; Enrique et al. (2016) 196(1_Supplement):209.23).

To begin the study, 11-week-old female NZB/WC57/BL6NJ mice (The Jackson Laboratories) were placed into 6 treatment groups of 10 mice each, in a balanced manner to achieve similar average body weight and proteinuria across groups. On Day 0, treatment with test articles began as outlined in Table E43 via intraperitoneal (IP) injection. Mice then received 1.5×10¹¹ virus particles of adenovirus expressing interferon alpha (AAV-IFNα; Vector Biolabs, Lot #230410 #1-7)) via intravenous tail vein injection on Day 0.

TABLE E43
Treatment, Dose Form, Dose Volume and Dose Frequency

| Group | n | Treatment | Dose (IP) | | Volume | | Frequency |
|---|---|---|---|---|---|---|---|
| 1 | 10 | Fc Control (AIS) | 8.3 | mg/kg | 5 | mL/kg | 2x/week |
| 2 | 10 | 26 TACI CRD2-Fc (AIS) | 10 | mg/kg | 5 | mL/kg | 2x/week |
| 3 | 10 | Telitacicept (RemeGen) | 12 | mg/kg | 5 | mL/kg | 2x/week |
| 4 | 10 | anti-mCD20 (rat IgG2b) | 0.25 | mg/dose | 200 | uL | 1x/week* |
| | | (BioLegend | | | | | |
| 5 | 10 | Efgartigimod (Argenyx) | 20 | mg/kg | 5 | mL/kg | 2x/week |
| 6 | 10 | Cyclophosphamide | 50 | mg/kg | 5 | mL/kg | 1x/week* |
| | | (TCI Chemicals) | | | | | |

*Mice in these groups also received vehicle (PBS) 1x per week to control for any stress from dosing 2x per week in the other group.

During the course of the study, body weight and proteinuria were measured 2× weekly and urinary creatinine was measured 1× weekly as readouts of disease. Proteinuria is a particularly important indicator of lupus nephritis severity, as elevated levels are associated with renal disease. Elevated urinary creatinine levels are also observed in lupus nephritis. In this study, urine dipsticks were used to measure levels of urinary protein (Chemstrip 2 GP, Roche Diagnostics Cat #11895397160) and creatinine (Accutest M2 Microalbumin Urine Reagent Strips, Jant Pharmacal Corp. Cat #UA502). Levels were measured as “scores” which correspond to color-coded changes on the dipstick to indicate concentrations of the analyte in the urine. A higher score equals a higher concentration of protein or creatinine in the urine.

For whole blood analyses, blood was collected via retro-orbital sinus into tubes containing EDTA for a complete hematological panel (measured at IDEXX Laboratories) and red blood cell (RBC) direct anti-globulin test (DAT) readouts at the following time points: Day −1 (2 mice/group) (hematological panel only), Day 42 (all mice), and terminal/Day 50 (all surviving mice). These assays provide readouts of the AIHA that can accompany lupus in patients, as well as lupus in the NZB/W lupus model. The RBC DAT assay measures levels of erythrocyte autoantibodies, which can lead to the RBC destruction, hemolysis, and anemia observed in AIHA. For serum analyses, whole blood was collected via the retro-orbital sinus into gel-clot activator tubes, serum isolated via centrifugation, and the serum stored at −80° C. until analyzed for anti-double-stranded (ds) DNA antibodies, which are high in lupus and a key readout for assessing disease severity and evaluating efficacy of therapeutics.

The RBC DAT assay was performed as follows: An aliquot (approximately 10 μL) of whole EDTA-collected blood was maintained at 4° C. until the following day when 3 μL from each mouse was plated into duplicate wells of a 96-well round bottom polystyrene assay plate (Corning 3797). Flow cytometry buffer (1×PBS with 0.5% BSA, 0.005% NaN3, 0.5 mM EDTA) was added and used to wash the cell pellet 2×(150-180 μL each). For all washes, the plate was centrifuged at 1700 g for 12 seconds to pellet cells and the supernatant flicked out prior to resuspension of pellets with flow buffer. To stain for RBC autoantibodies of various isotypes, the RBC pellets were resuspended in 50 μL of an antibody cocktail (anti-mouse IgG2c FITC [Southern Biotech 1079-02], anti-mouse IgM PerCP Cy5.5 [BioLegend 406512], anti-mouse IgA PE [Southern Biotech 1040-09], anti-mouse IgG2b PE-Cy7 [BioLegend 406714], anti-mouse pan Ig APC [Southern Biotech 1012-11], and anti-mouse IgG1 BV421 [BioLegend 406616], with all antibodies diluted 1:50 in flow buffer) and incubated at room temperature for 40 min with gentle shaking. The stained RBCs were washed 2× with flow buffer, resuspended in 100 μL flow buffer, and collected on a CytoFLEX LX flow cytometer (Becton-Coulter). The median fluorescence intensity (MFI) was calculated for each fluorochrome using FlowJo software version 10.8.1 (FlowJo LLC).

At the end of the study, after whole blood had been collected into EDTA for the hematological and RBC DAT readouts, mice were euthanized, and blood collected via cardiac puncture into tubes for isolation of serum. Mice were then perfused with PBS, and the left kidney, lacrimal glands, and submandibular glands (SMG) from each mouse collected, weighed (kidneys only), and fixed in 10% buffered formalin for histological analysis. Each of these tissues can become damaged as a result of lupus and other autoimmune diseases (i.e., Sjogren's Syndrome) and thus, histological analysis is of particular importance when evaluating the effect of therapeutics to reduce lupus and autoimmune disease manifestations. In addition, the right kidney of each mouse in the study was collected and weighed, and then embedded in OCT and stored at −80 C for analysis of glomerular IgG deposition, which is observed in many autoimmune renal diseases, including lupus nephritis. Spleens were also collected, weighed whole, and split into halves. One half was fixed in formalin for histological analysis and the other half placed into complete media (RPMI 1640 supplemented with 10% fetal calf serum, 0.1 mM MEM non-essential amino acids, 1 mM sodium pyruvate, 2 mM L-glutamine, 100 IU/mL penicillin, 100 g/mL streptomycin, 50 M 2-mercaptoethanol, 10 mM HEPES) for immunophenotyping by flow cytometry.

For histological preparation and analysis, kidneys were processed to paraffin blocks, and 1 periodic acid-Schiff (PAS) stained slide was prepared from each block (1 section per slide) and analyzed from each mouse. The scoring system was as follows: Glomerular lesions were analyzed for mesangial expansion, endocapillary proliferation, glomerular deposits, and extracapillary proliferation; tubular/interstitial lesions were analyzed for interstitial infiltrates, tubular atrophy, and interstitial fibrosis. All lesions were graded semi-quantitatively using a scoring system from 0 to 3 (i.e., 0: no changes; 1: mild changes; 2: moderate changes; 3: severe changes). Scoring of glomerulonephritis severity was performed blind, by a pathologist unaware of both treatment and readout results for each mouse.

For histological preparation and analysis of SMG and lacrimal gland, 2 SMGs and 2 lacrimal glands per mouse were each trimmed into 2 pieces, and all 4 pieces embedded into a single paraffin block. One (1) H&E section was prepared and analyzed for inflammation, scored as the number of inflammatory foci per mouse. Analysis was performed blind, by a pathologist unaware of both treatment and readout results for each mouse.

For histological preparation and analysis of spleen, the half spleen per mouse was embedded into a single paraffin block. One (1) H&E section was prepared and analyzed for the average diameter of 10 follicles, as well as assigned a follicle score from that ranged from 0 to 5 (i.e., 0: no white pulp (WP); 1: <10% WP; 2: 10 to 25% WP; 3: 25 to 50% WP; 4: 50 to 75% WP; 5: >75% WP). Analysis was performed blind, by a pathologist unaware of both treatment and readout results for each mouse.

The other half of the spleen was maintained in the RPMI complete media at 4° C. until the following day when RBC-lysed single cell suspensions were prepared and stained for flow cytometry analysis of immune cell subsets. An important mechanism of action of efficacious treatments for lupus is to reduce immune cell subsets that promote disease-causing autoantibodies and immune complexes, including (but not limited to) T follicular helper (T_{FH}) cells, germinal center (GC) B cells, and plasma cells. Flow cytometry analysis of the spleens in this study was performed using the following method: 1×10⁶ live cells were placed into a well of two 96-well plates (Corning, Cat. 3797), centrifuged at 1500×g for 10 seconds, the supernatant removed, and the cell pellet washed twice with Dulbecco's phosphate buffered saline (DPBS). The pellets were resuspended in 100 μL of live-dead stain (LIVE/DEAD Fixable Blue Dead Cell Stain Kit, Life Technologies Corp., 1:1500 dilution in DPBS) and incubated for 10 min in the dark at room temperature. Following two washes with flow buffer (175 μL each), pellets were resuspended in Mouse BD Fc Block (diluted 1:200 with flow buffer), and incubated in the dark for an additional 5 min at room temperature. Without any additional washes, 50 μL of a cocktail of the following flow cytometry antibodies (diluted in flow cytometry buffer) were added to each well of cells for the B cell panel or the T/regulatory B (Breg)/B1 cell panel. For the B cell panel, the following antibodies were combined for the cocktail: anti-mouse CD138 BV421 (BioLegend 142508; 1:50, final concentration), anti-mouse CD45 BV510 (BioLegend 103138; 1:100, final concentration), anti-mouse CD19 BV605 (BioLegend 115540; 1:100, final concentration), anti-mouse B220 BV785 (BioLegend 103246; 1:100, final concentration), anti-mouse CD95 FITC (BioLegend 152606; 1:100, final concentration), anti-mouse CD23 PerCP Cy5.5 (BioLegend 101618; 1:100, final concentration), anti-mouse GL7 PE-Cy7 (BioLegend 144620; 1:100, final concentration), anti-mouse TACI PE (BioLegend 133404; 1:50, final concentration), anti-mouse CD11b PE Dazzle (BioLegend 101256; 1:100, final concentration), anti-mouse CD21 APC (BioLegend 123412; 1:100, final concentration), anti-mouse IgD AF700 (BioLegend 405730; 1:100, final concentration), anti-mouse IgM APC Cy7 (BioLegend 406516; 1:100, final concentration), and anti-mouse CD38 BUV496 (Becton-Dickinson 741090; 1:100, final concentration). For the T/Breg/B1 cell panel, the following antibodies were combined for the cocktail: anti-mouse CD11b FITC (BioLegend 101206; 1:100, final concentration), anti-mouse CD3E BUV496 (Becton-Dickinson 612955; 1:100, final concentration), anti-mouse B220 BV785 (BioLegend 103246; 1:100, final concentration), anti-mouse IgD BV605 (BioLegend 405727; 1:100, final concentration), anti-mouse CD19 BV510 (BioLegend 115546; 1:100, final concentration), anti-mouse PD-1 BV421 (BioLegend 135221; 1:100, final concentration), anti-mouse CD21/35 PerCP Cy5.5 (BioLegend 123416; 1:100, final concentration), anti-mouse CD1d PE (BioLegend 123510; 1:100, final concentration), anti-mouse CXCR5/CD185 PE Dazzle (BioLegend 145521; 1:100, final concentration), anti-mouse CD5 PE-Cy7 (BioLegend 100622; 1:100, final concentration), anti-mouse CD23 AF700 (BioLegend 101632; 1:100, final concentration), anti-mouse CD4 APC (BioLegend 100412; 1:100, final concentration), and anti-mouse IgM APC-Cy7 (BioLegend 406516; 1:100, final concentration). The cells were incubated with one of the antibody cocktails in the dark, on ice, with gentle mixing for 45 min, followed by two washes with flow buffer (175 μL per wash). Cell pellets were resuspended in 200 μL flow buffer and collected on a CytoFLEX LX flow cytometer (Becton-Coulter). Data were analyzed using FlowJo software version 8.1.2 (FlowJo LLC, USA) and graphed using GraphPad Prism software (Version 10.8.1). Key cellular subset identification analysis included (but were not limited to) total B cells (CD45⁺ B220⁺CD19⁺ cells), germinal center (GC) B cells (CD45⁺ B220⁺, CD19⁺, GL7⁺, CD95⁺ cells), T follicular helper (T_{FH}) cells (CD45⁺, CD3⁺, CD4⁺, PD1⁺, CXCR5/CD185⁺ cells), plasmablasts (live TACI^{high}, CD138^{high}, B220⁺, CD19⁺ cells), plasma cells (live TACI^{high}CD138^{high} cells), early plasma cells (live TACI^{high}, CD138^{high}, B220⁻, CD19⁺ cells), and long-lived plasma cells (live TACI^{high}, CD138^{high}, B220⁻, CD19⁻ cells).

Statistically significant differences (p<0.05) between groups for all analyses were determined by one-way analysis of variance (ANOVA) and Fisher's least significant differences test (for normally distributed data), or the Kruskal-Wallis test and uncorrected Dunn's (for non-parametric data) using GraphPad Prism software (Version 9).

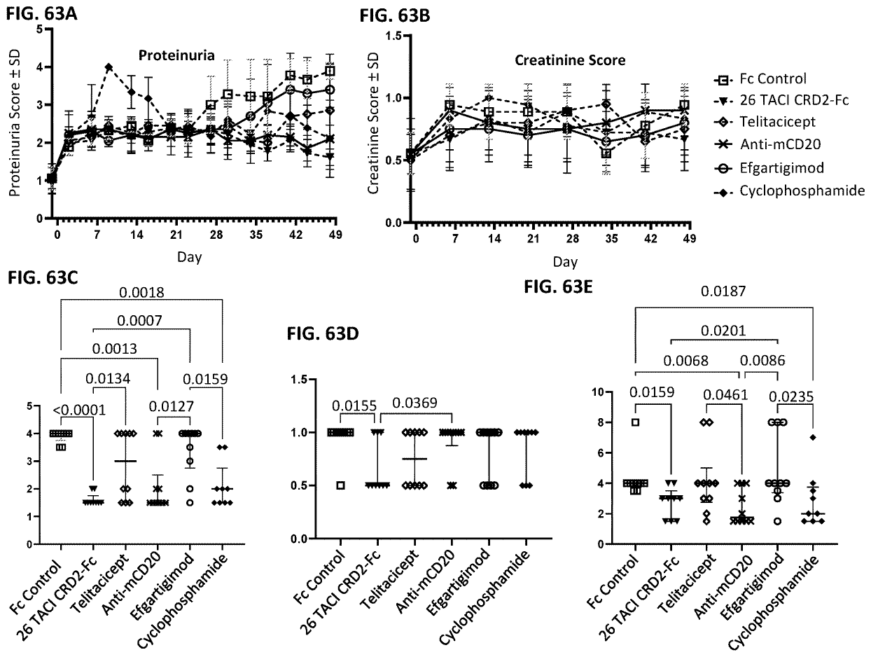

As shown in FIG. 63A, levels of proteinuria quickly rose in the model (i.e., by Day 3) as expected and continued to rise in the groups treated with cyclophosphamide, Fc control, efgartigimod, and to some extent, telitacicept. On the other hand, proteinuria scores did not continue to rise in mice treated with 26 TACI CRD2-Fc and tended to decrease during the last 2 weeks of the study. The last measurement prior to termination on Day 48 (FIG. 63C) indicated that 26 TACI CRD2-Fc-treated group had the lowest proteinuria scores amongst all groups, which were statistically significantly lower than those of the Fc control, telitacicept, and efgartigimod treatment groups. Urinary creatinine scores were also higher in the model (FIG. 63B), with 26 TACI CRD2-Fc again having the lowest creatinine scores, as well as one of the lowest proteinuria:creatinine scores, amongst all groups at Day 48 (FIGS. 63D-63E). Mice treated with 26 TACI CRD2-Fc had statistically significantly lower median values than the Fc control and anti-mouse CD20 treatment groups for urinary creatinine, and statistically significantly lower median values than Fc control and efgartigimod treatments for the urinary protein:creatinine ratio (FIGS. 63D-63E). These data show that treatment with 26 TACI CRD2-Fc can reduce important functional readouts of lupus nephritis and to a greater extent than relevant comparator therapeutics.

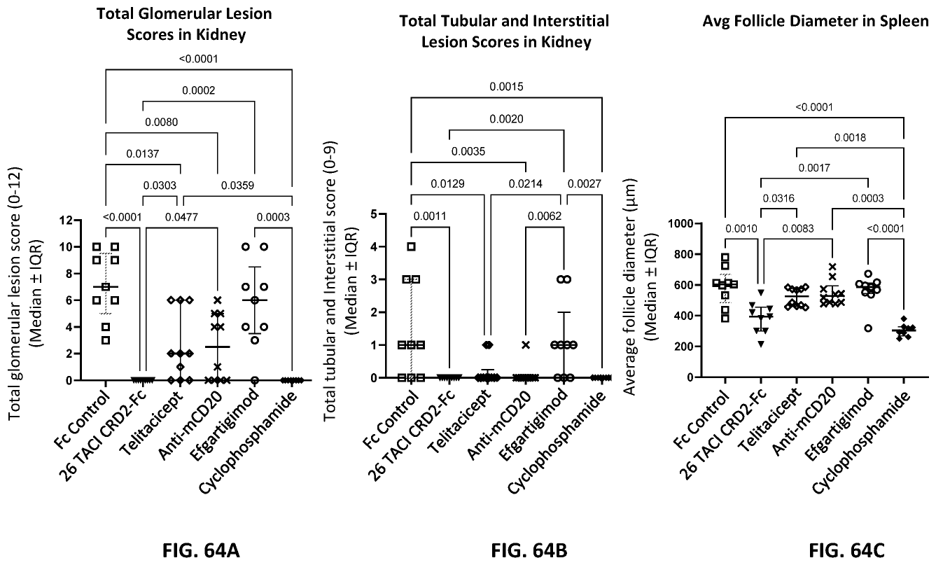

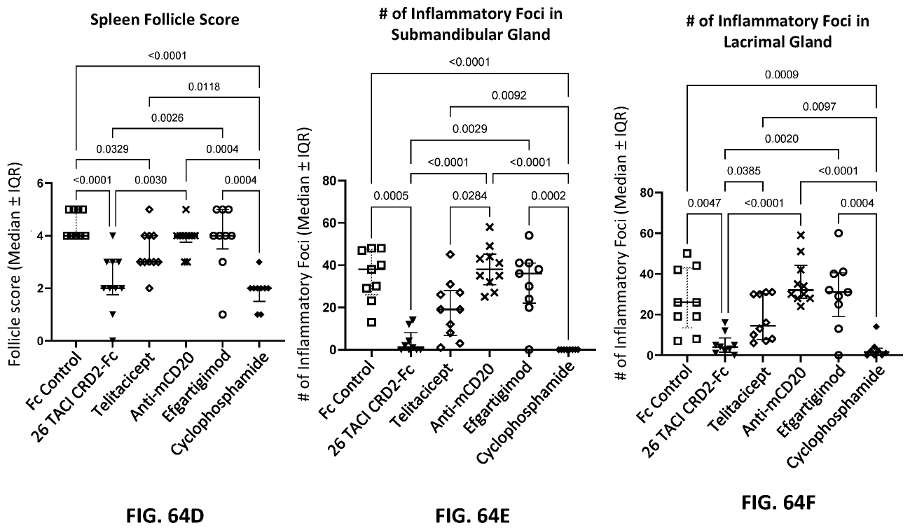

As shown in FIG. 64, the disease in this study resulted in histopathological damage to kidneys, spleen, submandibular glands, and lacrimal glands as expected. At the end of the study (Day 50), the group of mice treated with 26 TACI CRD2-Fc had scores that were lower than Fc control, telitacicept, anti-mouse CD20, and efgartigimod treatment groups, and were equally as low as the group treated with cyclophosphamide, a potent therapeutic used to treat patients with particularly severe cases of lupus. For the total glomerular lesion score (FIG. 64A), 26 TACI CRD2-Fc treatment resulted in histology scores that were statistically significantly lower than Fc control, telitacicept, anti-mouse CD20, and efgartigimod treatment groups, and were statistically significantly lower than Fc control and efgartigimod treatment groups for the total tubular and interstitial lesion scores in kidney (FIG. 64B), again indicating that 26 TACI CRD2-Fc was able to effectively reduce renal disease in lupus and to a greater extent than relevant comparator therapeutics. Spleen histopathology scores were also statistically significantly lower in the group treated with 26 TACI CRD2-Fc as compared to Fc control, telitacicept, and anti-mouse CD20 treatments (FIGS. 64C-64D). Submandibular histopathology scores were statistically significantly lower in the 26 TACI CRD2-Fc treated group as compared to Fc control, anti-mouse CD20, and efgartigimod-treated groups, and tended to be lower vs. the telitacicept group (FIG. 64E). Lacrimal gland histopathology scores were statistically significantly lower in the 26 TACI CRD2-Fc group as compared to Fc control, telitacicept, anti-mouse CD20, and efgartigimod treatment groups (FIG. 64F).

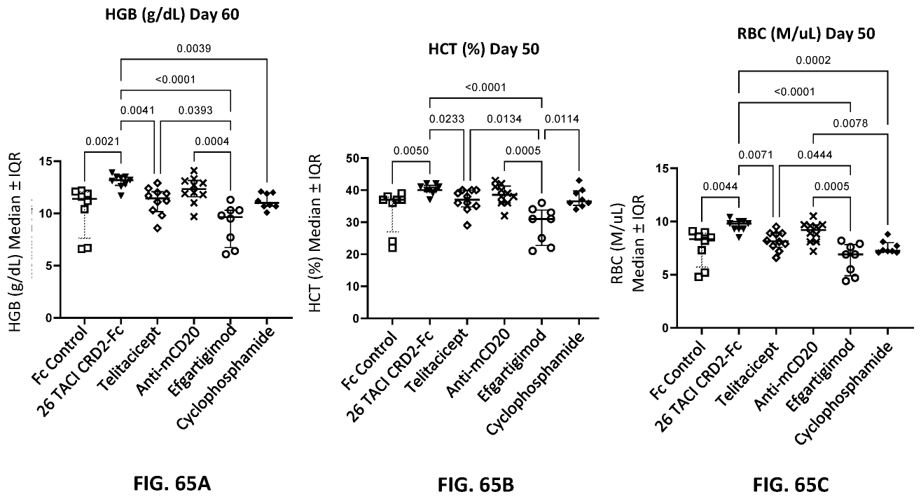

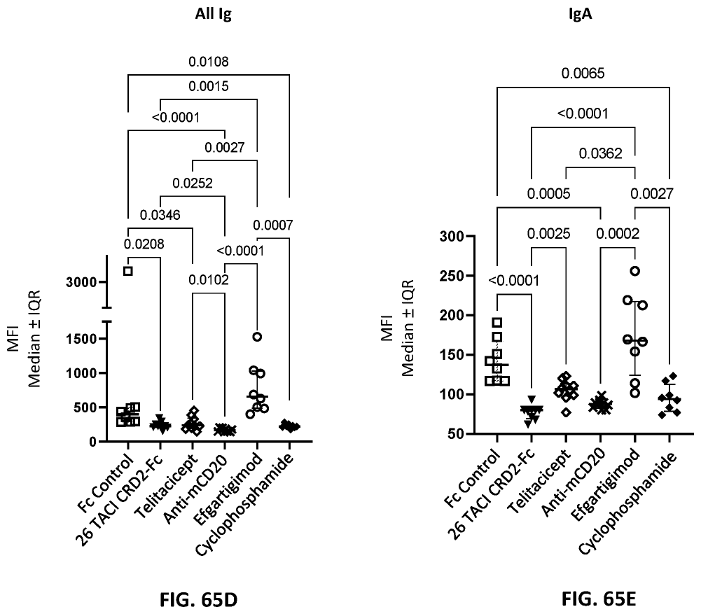

Readouts associated with anemia and/or AIHA are shown in FIGS. 65A-65E with terminal (Day 50) hemoglobin, hematocrit, and RBC levels, as well as RBC DAT levels exemplified. Signs of anemia/AIHA were present in this study, as shown by reduced hemoglobin, hematocrit, and RBC levels in the Fc control-treated group (FIGS. 65A-65C). The group of mice treated with 26 TACI CRD2-Fc did not present with signs of anemia, as shown by this group having the most consistently high levels of hematocrit, hemoglobin, and RBCs amongst all groups, with statistically significantly higher values as compared to Fc control, telitacicept, and efgartigimod treatment groups for each of these readouts (FIGS. 65A-65C). Levels of RBC DAT were also elevated in the model, with pan Ig DAT levels being statistically significantly lower in the group treated with 26 TACI CRD2-Fc as compared to Fc control and efgartigimod treatment groups (FIG. 65D), and levels of IgA DAT being the lowest amongst all groups with 26 TACI CRD-Fc treatment (statistically significantly lower as compared to Fc control, telitacicept, and efgartigimod) (FIG. 65E). Taken together, these results show that 26 TACI CRD2-Fc treatment was effective in reducing the anemia/AIHA and erythrocyte autoantibodies that can occur in lupus and to a greater extent than relevant comparator therapeutics.

Immune cell subsets that are of particular pathogenic importance to autoimmune and inflammatory diseases, including lupus nephritis, were also evaluated in this study in spleens collected at the end of the study (data not shown). Treatment of mice with 26 TACI CRD2-Fc potently reduced total B cells, B cell subsets that are important for antibody production (i.e., GC B cells, transitional-2 cells, follicular B cells, marginal zone B cells, plasmablasts, plasma cells, early plasma cells, long-lived plasma cells), and T_{FH} cells (also important for antibody production) to levels that were lower than all other treatment groups.

At the end of the study, whole blood was collected via cardiac puncture into gel-clot activator tubes for isolation of serum via centrifugation, and stored at −80° C. until used to measure the accumulation of urea nitrogen in blood (BUN), which is an indicator of renal function. Higher levels of BUN have been associated with lupus nephritis. Analysis of BUN was performed using the Urea Nitrogen Colorimetric Detection Kit (Thermo Fisher Scientific, #EIABUN). Samples were run in unicate and plates read at 450 nm. A standard curve was used to convert the optical density to mg/dL (concentration). Higher concentrations of BUN are associated with worsening of renal function.

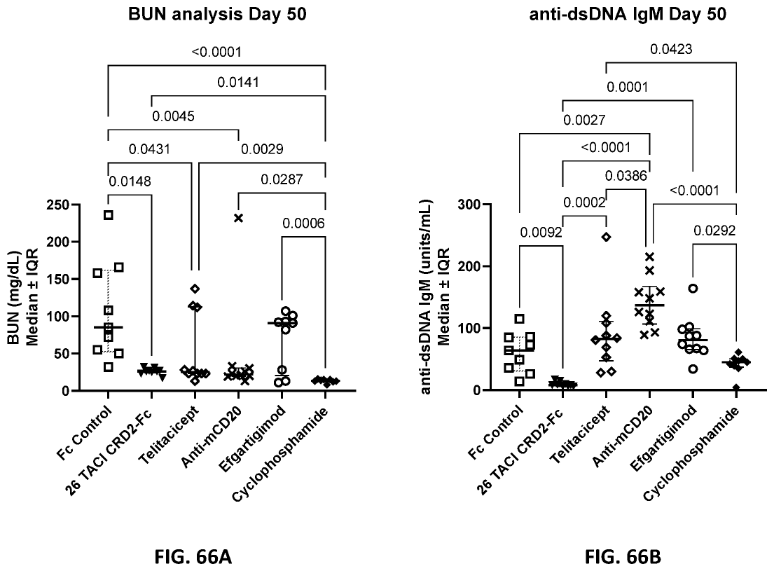

The serum BUN levels at termination, as shown in FIG. 66A, indicate that mice treated with 26 TACI CRD2-Fc had statistically significantly lower median BUN levels compared to Fc control. The reduction in serum BUN levels in the 26 TACI CRD2-Fc group was similar, but with less variability, when compared to telitacicept, anti-mCD20, and efgartigimod. These data indicate that treatment with 26 TACI CRD2-Fc can improve important functional readout of renal function during lupus nephritis with similar efficacy compared to the relevant clinical comparator therapeutics.

For terminal serum analyses, whole blood was collected via cardiac puncture into gel-clot activator tubes, isolated via centrifugation, and the serum stored at −80° C. until analyzed for anti-double-stranded (ds) DNA antibodies, which are high in lupus and a key readout for assessing disease severity and evaluating efficacy of therapeutics. The detection of anti-dsDNA antibodies was performed using an ELISA performed at Hooke Laboratories. The ELISA plates were coated with poly-L-lysine followed by dsDNA. Plates were blocked with 3% BSA in PBS. Each serum sample was tested at 4 dilutions in unicate in 1% BSA in PBS: 1:500, 1:1000, 1:2000, and 1:4000. Standards (control mouse serum) were serially diluted in 1% BSA in PBS and run in duplicate. Antibodies were detected using HRP-conjugated mouse IgM Fc binding protein (1:2000, Novus, catalog #NB7497). Plates were read at 450 nm. A standard curve was used to convert the optical density to units/dL.

As shown in FIG. 66B, treatment with 26 TACI CRD2-Fc resulted in the lowest level of anti-dsDNA IgM amongst all groups and was statistically significantly lower than Fc control, telitacicept, anti-mCD20, and efgartigimod. These data indicate that 26 TACI CRD2-Fc was able to effectively reduce the levels of anti-dsDNA autoantibodies in lupus and to a greater extent than relevant comparator therapeutics.

### Example 33. Assessment of the Activity of TACI-Fc Fusion Protein in a Mouse Model of Epidermolysis Bullosa Acquisita (EBA)

This Example describes the assessment of 26 TACI CRD2-Fc and an Fc isotype control to affect disease in vivo in a highly relevant mouse model of epidermolysis bullosa acquisita (EBA), an autoimmune blistering skin disease (Kasprick et al, Curr Protoc Pharmacol (2019) 84:e55). This disease in patients, as well as in the mouse model evaluated, is characterized and caused by autoantibodies targeting type VII collagen (COL7). After binding of the autoantibodies to their target antigen in the skin and mucous membranes, chronic inflammation and often scarring, imposes a high burden on affected patients. EBA is extremely difficult to treat and there is a very high medical need to reduce disease and disease relapses. Because B cells and autoantibodies play a key role in the pathogenesis of EBA, 26 TACI CRD2-Fc treatment could reduce disease progression and/or pathogenic antibodies in EBA via its ability to reduce B cells and autoantibody levels.

To begin the study, susceptible B6.SJL-H2s C3c/1CyJ (B6.s) mice were immunized with 120 μg of von-Willebrand-factor A-like domain 2 (vWFA2, domain of COL7; mCOL7^{vWFA2}) emulsified (1:1) in TiterMax®. From Week 4 to 10 after antigen injection, mice were monitored for clinical disease severity by examining for skin lesions, and different body parts individually scored by the appearance of crust, erythema, lesions and/or alopecia. Mice that developed symptoms of EBA on 2% or more of their body surface area were randomly allocated to receive either the 26 TACI CRD2-Fc (10 mg/kg, 2× weekly, IP injection) or molar matched level of Fc control (8.3 mg/kg, 2× weekly, IP) for 10 weeks; clinical scoring continued and serum was collected half-way through treatment (5 weeks) for determination of total and antigen-specific antibodies. At the end of the treatment period, mice were euthanized, and serum collected for measurements of antibodies, skin harvested for histopathology (i.e., H&E staining) and IHC (i.e., detection of tissue-bound IgG and deposition of the complement component C3), and lymph nodes and bone marrow collected for analysis of immune cell subsets by flow cytometry (i.e., total cell subsets and antigen specific [mCOL7^{vWFA2}])

Serum levels of total mouse IgM, IgA and IgG were determined by enzyme-linked immunosorbent assay (ELISA) using mouse quantification sets (Cat. #: IgA: E90-103; IgG1: E90-105; IgG2a: E90-107-38; IgG2b: E90-109; IgG2c: E90-136; IgG3: E90-111; IgM: E90-101; Bethyl, Montgomery, Texas, USA) following the manufacturer's protocol. For detection of mouse anti-mCOL7^{vWFA2} antibodies, the same kits were used following this modification (Pipi et al, J Invest Dermatol (2022) 142(6):1552-1564): Each well of a 96-well ELISA plate was coated with 250 ng recombinant mCOL7^{vWFA2}. After blocking, diluted samples were added and incubated for 60 min. Bound antibodies were detected by HRP-conjugated goat anti-mouse antibodies (Bethyl) and tetramethylbenzidine (Invitrogen, Waltham, MA, USA). The enzymatic color reaction was stopped by 2 M sulfuric acid (Carl Roth, Karlsruhe, Germany), and the change in OD was measured with a GloMax® Discover Microplate Reader photometer (Promega, Walldorf, Germany) at 450 nm. Standard reference curves were established by using the provided mouse reference sera (Bethyl).

For flow cytometric measurements, single-cell suspensions of both right and left inguinal and popliteal lymph nodes, as well as bone marrow cells from a pool of both femora and tibiae were prepared. Suspensions (2×10⁶ cells for each panel) were analyzed follows: Live/dead staining was performed with fixable viability dye 510 (BD Bioscience) 1:1000 in DPBS for 15 min (room temperature), followed by 2 washes with flow cytometry buffer (0.5% BSA, 25 mM EDTA in Dulbecco's PBS), incubation with Fc receptor blockade for 10 minutes at room temperature (Miltenyi Biotec; 1:10 dilution in flow cytometry buffer), and incubation with cocktails of flow cytometry antibodies for B cell subsets (20 minutes at 4 C). Stained samples (200 μL) were collected on a MACSQuant Analyzer 10 (Miltenyi Biotec) and data analyzed using MACSQuantify software.

The flow antibodies used were: PE anti-mouse CD267 (TACI) antibody (Biolegend), APC anti-mouse IgD antibody (Biolegend), BV421 anti-mouse IgD antibody (Biolegend), PE anti-mouse CD23 antibody (Biolegend), PerCP/Cy5.5 anti-mouse/human CD45R/B220 antibody (Biolegend), PE/Cy7 anti-mouse CD38 antibody (Biolegend), APC anti-mouse CD21/CD35 (CR2/CR1) antibody (Biolegend), APC/Cy7 anti-mouse IgM antibody (Biolegend), BV421 anti-mouse CD138 (Syndecan-1) antibody (Biolegend), PE/Cy7 anti-mouse CD95 (Fas) antibody (Biolegend), APC anti-mouse/human GL7 antibody (Biolegend), PE/Cy7 anti-mouse IgM antibody (Biolegend), APC-Vio770 anti-mouse CD19 (Miltenyi Biotec), and Alexa Fluor 488-conjugated mCOL7vWFA2 (labeling performed using Alexa Fluor 488 Monoclonal Antibody Labeling Kit [Invitrogen] according to the manufacturer's instructions).

Immune cell subsets were determined using the following markers (gated first on live cells): Total B Cells (B220+CD19+); T1 B Cells (B220+IgM+CD23-CD21^{low}); T2 B Cells (CD45+B220+IgM+CD23+CD21^{hi} IgM hi); Marginal Zone B Cells (CD45+B220+IgM+CD23−CD21+); Follicular B Cells (CD45+B220+IgM+CD23+CD21^{var}); Class Switched B Cells (B220+CD19+IgM−IgD−CD138− [bone marrow only]); GC B Cells (B220+CD19+GL7+CD95+); Plasma Cells (CD138+TACI+); Early Plasma Cells (CD138+TACI+CD19+B220−); Long Lived Plasma Cells (CD138+TACI+CD19−B220−); Plasmablasts (CD138+TACI+CD19+B220+); Mature B Cells (B220+CD19+IgM+IgD+).

Overall, clinical EBA scores over the 10 weeks of treatment were not significantly affected by treatment with 26 TACI CRD2-Fc as compared to Fc control treatment (data not shown). However, the pathogenesis of EBA is characterized by several morphological and immunological changes in the skin which were analyzed in more detail via histopathological, as well as serum levels of antibodies (total and antigen-specific) and immune cell subsets (total and antigen-specific).

Treatment with 26 TACI CRD2-Fc statistically significantly reduced the cumulative skin H&E score (FIG. 1), which included key characteristics that are elevated in EBA, such as inflammatory cell infiltration, epidermal thickness, and split formation at the dermal-epidermal junction. Although there was no statistically significant reduction in IgG or C3 deposition at the dermal-epidermal junction, there was less staining below the skin surface (data not shown).

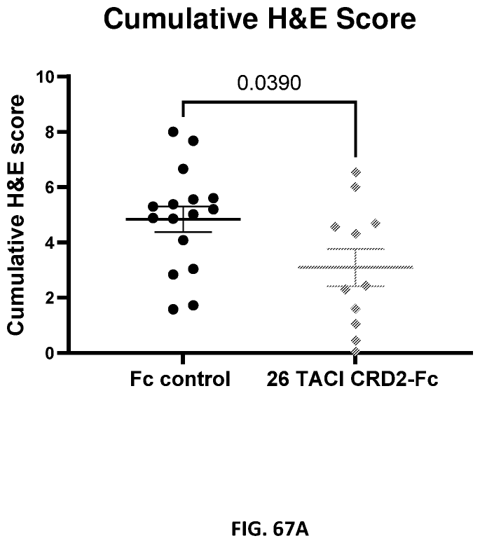

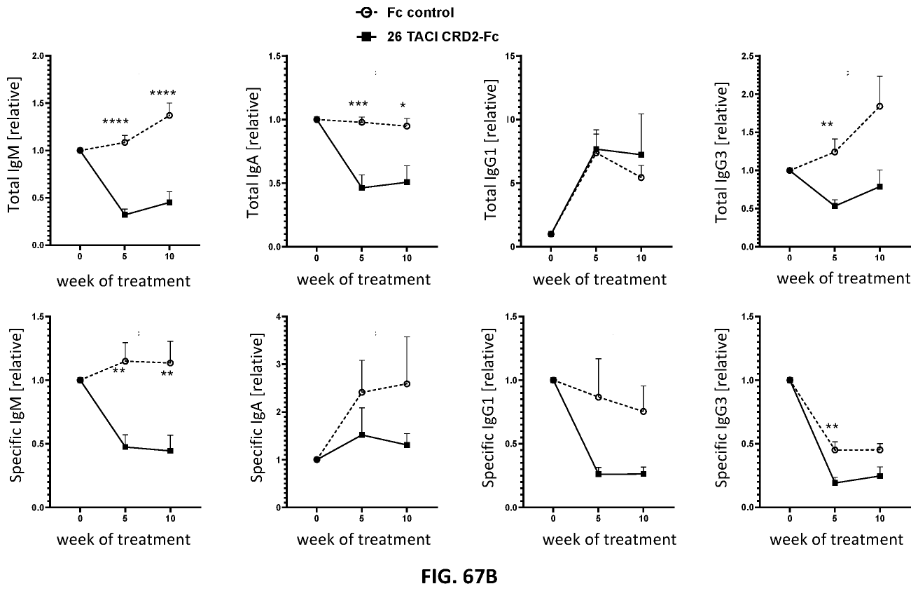

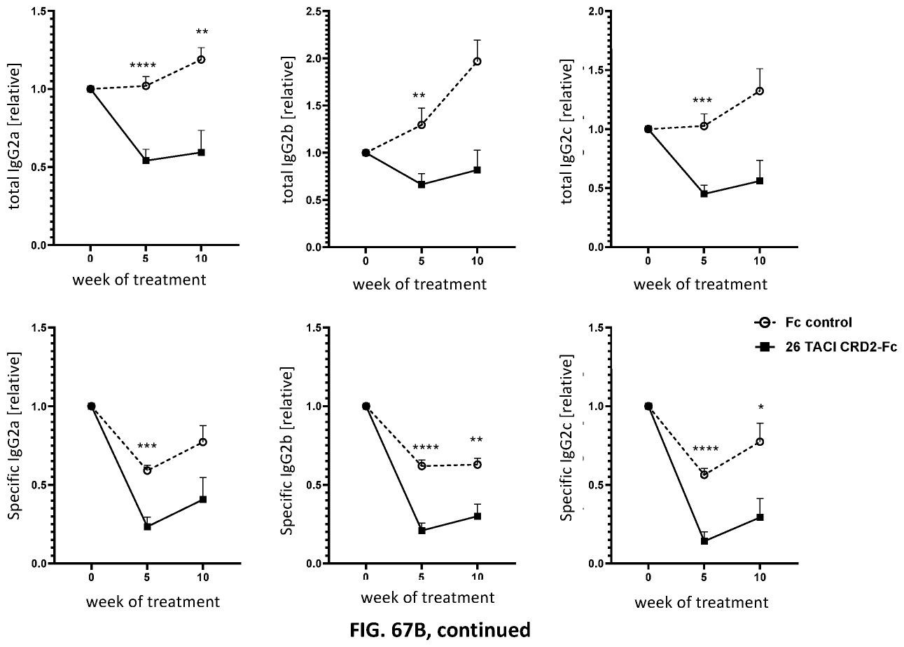

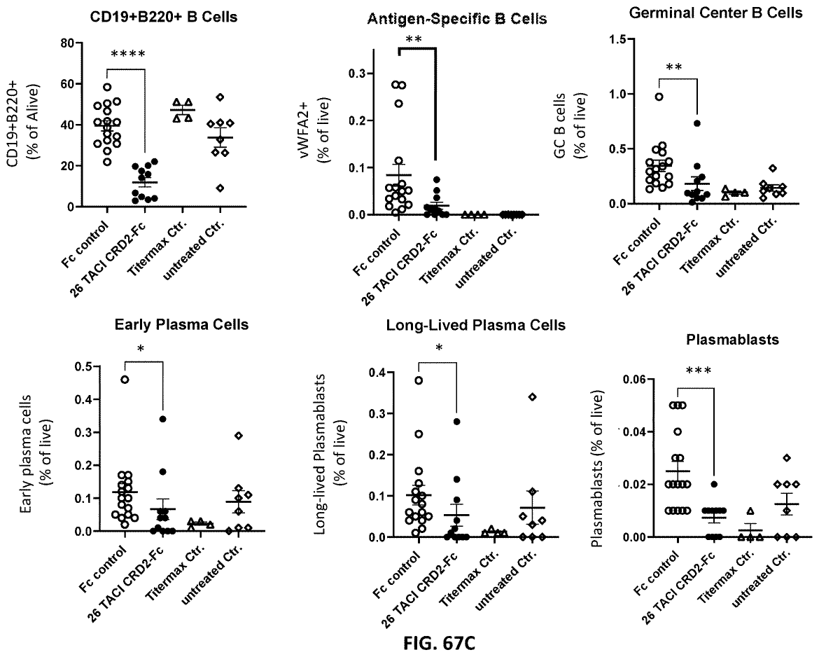

Serum concentrations of total IgM, IgA, IgG2a-c, as well as mCOL7^{vWFA2}-specific IgM, IgG2a, IgG2b, IgG2c, and IgG3 were statistically significantly lower in the group of mice treated with 26 TACI CRD2-Fc as compared to Fc control, and total IgG1, mCOL7^{vWFA2}-specific IgA and -IgG1 concentrations tended to be lower with 26 TACI CRD-Fc treatment (FIG. 67B). This is important for EBA pathogenesis since it is driven by autoantigen-specific IgG2 antibodies and thus, this reduction of mCOL7^{vWFA2}-specific IgG2a-c is clinically relevant (Sitaru et al, J Immunol Baltim Md (2006) 1950(177):3461-3468). The tendency for lower levels of COL7-specific IgA autoantibodies is important as these pathogenic IgA autoantibodies are present in approximately a third of patients with EBA (Recke et al, J Immunol (2014) 193:1600-1608; Buijsrogge et al, J Dermatol (2011) 165:92-98).

Flow cytometric analysis of lymph nodes indicated that compared with Fc control treatment, treatment with 26 TACI CRD2-Fc resulted in statistically significantly lower percentages of total B cells, B cell subsets (T1 B cells, T2 B cells, marginal zone B cells, follicular B cells, germinal center B cells), and plasma cells (i.e., plasmablasts, early plasma cells, and long-lived plasma cells) (FIG. 67C). Importantly, 26 TACI CRD2-Fc resulted in statistically significantly lower mCOL7^{vWFA2}-specific B cells in the lymph nodes as compared to Fc control treatment. In the bone marrow, percentages of total B cells, mCOL7^{vWFA2}-specific B cells, class-switched B cells, and plasmablasts were comparable between 26 TACI CRD2-Fc- and Fc control-treated mice, whereas the amounts of mature B cells as well as early and long-lived plasma cells were significantly decreased by 26 TACI CRD2-Fc treatment.

In summary, 26 TACI CRD2-Fc treatment in a mouse model of EBA in mice resulted in statistically significantly lower total B cells, B cell subsets, and mCOL7^{vWFA2}-specific B cells in lymph nodes and, thus, inhibited production of antibodies in general. In addition, treatment with 26 TACI CRD2-Fc statistically significantly reduced antigen-specific antibodies of the IgM, IgG3 and IgG2a-c subclasses and cumulative histopathological skin scores as compared to Fc control treatment (FIGS. 67A and 67C).

### Example 34. Assessment of the Activity of TACI-Fc Fusion Protein in a Mouse Model of Autoimmune Encephalitis

This Example describes the assessment of 26 TACI CRD2-Fc and an Fc isotype control to affect disease in vivo in a mouse model of anti-N-methyl-D-aspartate receptor (NMDAR) autoimmune encephalitis (AIE). This disease in patients, as well as in the mouse model evaluated, is characterized and caused by autoantibodies targeting NMDAR and glutamate receptor ionotropic-1 (GluN1). There is a high medical need for efficacious therapies to treat these patients, who present with psychosis, dyskinesia, and seizures. Because autoantibodies play a key role in the pathogenesis of AIE, 26 TACI CRD2-Fc treatment could reduce disease progression and/or pathogenic antibodies via its ability to reduce B cells and autoantibody levels.

To begin the study, 8-week-old female C57BL/6NJ mice (n=16) were immunized subcutaneously with 0.2 mL of an emulsion that contained 0.2 mg of a disease-causing GluN1 peptide (GluN1356_385; Biomatek) (Ding et al, J Neuroinflammation (2021) 18(1):53), emulsified (1:1) in incomplete Frend's adjuvant (IFA; Sigma-Aldrich) supplemented with Mycobacterium tuberculosis H37Ra (0.4 mg per mouse; Becton-Dickinson Difco). Mice were boosted 4 and 8 weeks later with the same emulsion except without Mycobacterium tuberculosis. Immediately following the boost at 8 weeks and then 48 hours later, mice were administered pertussis toxin (0.2 ng, IP injection).

Serum levels of anti-GluN1 antibodies were determined by enzyme-linked immunosorbent assay (ELISA) 4 days prior to the Week 4 boost (i.e., Week 3.5) and again at termination (Week 10) using an assay developed at Alpine Immune Sciences. Wells of a 96-well ELISA plate was coated with 1 g/mL GluN1₃₅₆₋₃₈₅. After blocking, diluted serum samples were added and incubated for 60 min. Bound antibodies were detected by HRP-conjugated goat anti-mouse IgG (Jackson ImmunoResearch) and tetramethylbenzidine (SeraCare). The enzymatic color reaction was stopped by 1 M H₃PO₄, and the change in OD was measured using a Spectra Max iD3 (Molecular Devices) microplate reader at 450 nm.

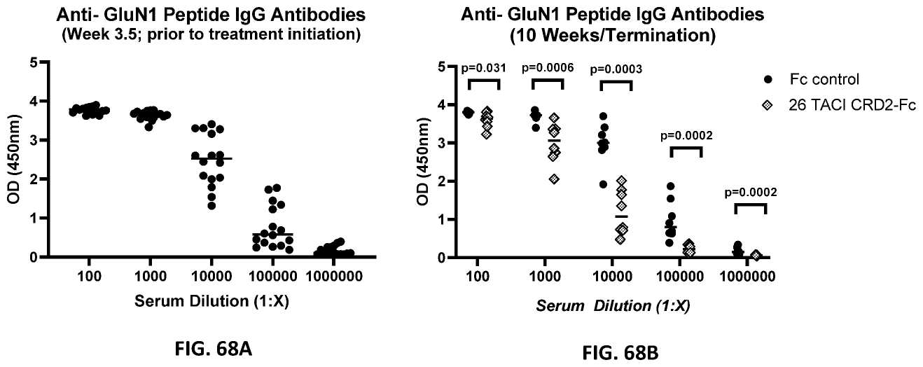

At the time of the boost at Week 4, when serum anti-GluN1 peptide IgG levels were present at even the 1:100,000 dilution (FIG. 68A), treatment with 26 TACI CRD2-Fc (10 mg/kg; IP; dosed 2× weekly) or molar-matched levels of Fc control (8.3 mg/kg; IP; dosed 2×weekly) began, with mice being placed in groups in a balanced manner, such that mice in each treatment group had similar mean body weights and levels of anti-GluN1 peptide antibodies. Treatments continued until Week 9.5 of the model (i.e., 1.5 weeks after the final GluN1 peptide immunization) and mice were then euthanized at Week 10. Terminal collections included serum for anti-GluN1 peptide IgG antibodies and brains (embedded and frozen in OCT) for IHC analysis of mouse IgG/total IgG antibodies.

Treatment with 26 TACI CRD2-Fc resulted in statistically significantly lower serum anti-GluN1 peptide antibodies compared to Fc control-treated mice at termination (FIG. 68B) at each of the serum dilutions. In patients and in mouse models, serum levels of these pathogenic antibodies are associated with pathogenic antibody deposition in the central nervous system, which in turn cause disease symptoms (Braczkowski et al, Diagnostics (2023) 13(15:2589). Thus, statistically significantly lower serum anti-GluN1 IgG antibodies would result in reductions in the antibodies in brains and an overall reduction in disease.

Taken together, these data indicate that treatment with 26 TACI CRD2-Fc can statistically significantly reduce pathogenic antibodies in a model of anti-NMDAR AIE and would thus be anticipated to be an effective treatment of patients with anti-NMDAR AIE.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

What is claimed:

1. A method of treating an autoantibody-related disease or disorder in a subject, the method comprising administering to the subject a TACI-Fc fusion protein that is a homodimer of two polypeptides of the formula TACI-linker-Fc, wherein TACI is a variant TACI polypeptide comprising one or more amino acid substitutions selected from the group consisting of K77E, F78Y and Y102D in the amino acid sequence set forth in SEQ ID NO:13, and wherein the TACI-Fc fusion protein is administered subcutaneously at a dose of from at or about 80 mg to at or about 480 mg once every four weeks (Q4W).

2. A method of treating an autoantibody-related disease or disorder in a subject, the method comprising administering to the subject a TACI-Fc fusion protein that is a homodimer of two polypeptides of the formula TACI-linker-Fc, wherein TACI is a variant TACI polypeptide comprising one or more amino acid substitutions selected from the group consisting of K77E, F78Y and Y102D in the amino acid sequence set forth in SEQ ID NO:13, and wherein the TACI-Fc fusion protein is administered subcutaneously at a dose of from at or about 24 mg to at or about 480 mg once every four weeks (Q4W), once every eight weeks (Q8W), or once every twelve weeks (Q12W).

3. The method of claim 1, wherein the variant TACI polypeptide comprises the amino acid substitutions K77E, F78Y and Y102D.

4. The method of any of claims 1-3, wherein the dose is from at or about 80 mg to at or about 240 mg Q4W.

5. The method of any of claims 1-4, wherein the dose is at or about 80 mg Q4W.

6. The method of any of claims 1-4, wherein the dose is at or about 240 mg Q4W.

7. The method of claim 2, wherein the dose is from at or about 24 mg to at or about 240 mg once every four weeks (Q4W), once every eight weeks (Q8W), or once every twelve weeks (Q12W).

8. The method of claim 2 or claim 3, wherein:
(i) the dose is at or about 24 mg Q4W;
(ii) the dose is at or about 24 mg Q8W;
(iii) the dose is at or about 24 mg Q12W;
(iv) the dose is at or about 80 mg Q8W;
(v) the dose is at or about 80 mg Q12W;
(vi) the dose is at or about 240 mg Q8W;
(vii) the dose is at or about 240 mg Q12W.

9. The method of any of claims 1-8, wherein the autoantibody-related disease or disorder is selected from the group consisting of a rheumatic disease or disorder, a renal (kidney) disease or disorder, a hematologic disease or disorder, a dermatologic disease or disorder, or a neurologic disease or disorder.

10. The method of any of claims 1-9, wherein the autoantibody-related disease or disorder is a rheumatic disease or disorder.

11. The method of any of claims 1-10, wherein the autoantibody-related disease or disorder is Sjogren's.

12. The method of any of claims 1-10, wherein the autoantibody-related disease or disorder is Systemic lupus erythematosus (SLE).

13. The method of any of claims 1-12, wherein the TACI-Fc fusion protein reduces the amount of circulating immunoglobulin G (IgG).

14. The method of claim 13, wherein circulating IgG is reduced by at least 10% from the subject's baseline, optionally about 35% from the subject's baseline.

15. The method of any of claims 1-14, wherein the TACI-Fc fusion protein does not result in severe hypogammaglobulinemia in the subject.

16. The method of any of claims 1-15, wherein, among a plurality of subjects treated by the method, the TACI-Fc fusion protein results in severe hypogammaglobulinemia in less than 5% of the treated subjects, optionally less than 3% of the subjects, and more optionally less than 1% of the treated subjects.

17. The method of claim 15 or claim 16, wherein severe hypogammaglobulinemia is characterized by circulating IgG<3 g/L, optionally circulating IgG<1.5 g/L or more optionally circulating IgG<1.0 g/L.

18. The method of any of claims 1-12 and 14, wherein administration of the TACI-Fc fusion protein does not reduce circulating IgG to <1.5 g/L in the subject.

19. The method of any of claims 1-12 and 14, wherein administration of the TACI-Fc fusion protein does not reduce circulating IgG to >1.0 g/L in the subject.

20. A method of treating Systemic lupus erythematosus (SLE), the method comprising:
a) selecting a subject for administration of a TACI-Fc fusion protein that has been diagnosed with SLE; and
b) administering to the selected subject the TACI-Fc fusion protein, wherein:
the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
the TACI-Fc fusion protein is administered subcutaneously at a dose of from at or about 80 mg to at or about 480 mg once every four weeks.

21. The method of claim 20, wherein the dose is from at or about 80 mg to at or about 240 mg Q4W.

22. The method of claim 20 or claim 21, wherein the dose is at or about 80 mg Q4W.

23. The method of claim 20 or claim 21, wherein the dose is at or about 240 mg Q4W.

24. The method of any of claims 12-23, wherein the systemic lupus erythematosus is mild to moderate systemic lupus erythematosus or moderate to severe systemic lupus erythematosus.

25. The method of any of claims 12-24, wherein the subject is selected for treatment if at the time of screening the subject has active SLE for ≥6 months.

26. The method of any of claims 12-25, wherein the subject is selected for treatment if at the time of screening the SLE is characterized by one or more of the following:
(i) a hybrid SELENA-SLEDAI score ≥8 or a hybrid SELENA-SLEDAI ≥6 if there is high anti-dsDNA or low complement (C) levels;
(ii) ≤6 g/g urine total protein to creatinine ratio (proteinuria);
(iii) A grade in the BILAG score in ≥1 organs;
(iv) B grade in the BILAG score in ≥2 organs; and
(v) Physicians Global Assessment (PGA) score ≥1.0.

27. The method of any of claims 12-26, wherein the subject is receiving standard therapy for treating the SLE.

28. The method of claim 12-27, wherein the subject is selected for treatment if the at the time of screening or the time of administering the TACI-Fc fusion protein, the subject is receiving a stable standard treatment regimen characterized by the stable use of a standard therapy for treating the SLE, optionally wherein the stable use is stable use of the standard therapy for at least 30 days.

29. The method of any of claims 12-27, wherein the TACI-Fc fusion protein is administered to the subject in combination with a standard therapy for treating the SLE.

30. The method of any of claims 27-29, wherein the standard therapy comprises one of more of a corticosteroid, antimalarial (e.g. hydroxychloroquine), an non-steroidal anti-inflammatory drug (NSAID), or an immunosuppressant or immunomodulator, or any combination thereof, optionally wherein the immunosuppressant or immunomodulator is selected from the group consisting of including azathioprine, mycophenolate (e.g. mycophenolate mofetil or sodium mycophenolate), cyclophosphamide, methotrexate, leflunomide, tacrolimus, cyclosporine and combinations of any of the foregoing.

31. The method of any of claims 27-30, wherein the standard therapy comprises a corticosteroid and administration of the corticosteroid is tapered after administering the TACI-Fc fusion protein.

32. The method of any of claims 12-29, wherein the SLE is severe SLE.

33. The method of any of claims 12-32, wherein the subject is selected for treatment if at the time of screening the subject is characterized by one or more of the following:
(i) severe lupus nephritis, optionally defined as urine protein >6 g/24 hours or serum creatinine >2.5 mg/dL or 221 μmol/L;
(ii) required hemodialysis;
(iii) received high-dose corticosteroids for ≥14 days in the last 2 months, optionally wherein the high-dose corticosteroid is treatment with prednisone >100 mg/day or equivalent; and
(iv) central nervous system disease caused by SLE or not caused by SLE in the last 2 months; optionally wherein the central nervous system disease is epilepsy, psychosis, organic brain syndrome, cerebrovascular accident, encephalitis, or central nervous system vasculitis.

34. The method of any of claims 1-9, wherein the autoantibody-related disease or disorder is a renal (kidney) disease or disorder.

35. The method of any of claims 1-9 and 32, wherein the autoantibody-related disease or disorder is a Glomerulonephritis.

36. A method of treating a Glomerulonephritis, the method comprising:
a) selecting a subject for administration of a TACI-Fc fusion protein that has been diagnosed with glomerulonephritis; and
b) administering to the selected subject the TACI-Fc fusion protein, wherein:
the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
the TACI-Fc fusion protein is administered subcutaneously at a dose of from at or about 80 mg to at or about 480 mg once every four weeks.

37. The method of claim 36, wherein the dose is from at or about 80 mg to at or about 240 mg Q4W.

38. The method of claim 36 or claim 37, wherein the dose is at or about 80 mg Q4W.

39. The method of claim 36 or claim 37, wherein the dose is at or about 240 mg Q4W.

40. The method of any of claims 36-39, wherein the subject is selected for treatment if at the time of screening the subject has active Glomerulonephritis.

41. The method of any of claims 36-40, wherein the Glomerulonephritis is selected from the group consisting of IgA Nephropathy, Lupus Nephritis and Primary Membranous Nephropathy.

42. The method of any of claims 36-41, wherein the Glomerulonephritis is IgA Nephropathy and the subject is selected for treatment if at the time of screening the subject is characterized by one or both of the following:
(i) the subject was diagnosed with IgA Nephropathy ≤5 years prior to the screening; and
(ii) ≥0.75 g/g urine total protein to creatinine (proteinuria).

43. The method of any of claims 36-41, wherein the Glomerulonephritis is IgA Nephropathy and the subject is selected for treatment if at the time of screening the subject is characterized by one or more of the following:
(i) the subject was diagnosed with IgA Nephropathy ≤5 years prior to the screening;
(ii) ≥0.75 g/g urine total protein to creatinine (proteinuria); and
(iii) elevated galactose deficient IgAQ1 (Gd-IgA1).

44. The method of claim 43, wherein the TACI-Fc fusion protein reduces Gd-IgA1.

45. The method of claim 44, wherein Gd-IgA1 is reduced by more than 50%.

46. The method of any of claims 36-41, wherein the Glomerulonephritis is Lupus Nephritis and the Lupus Nephritis is characterized as Class III (active focal), Class IV (diffuse) and/or Class V (lupus membranous nephropathy).

47. The method of any of claims 36-41 and 46, wherein the Glomerulonephritis is Lupus Nephritis and the subject is selected for treatment if at the time of screening the subject is characterized by one or more of the following:
(i) the subject was diagnosed with Lupus Nephritis Class II-V≤3 years prior to the screening;
(ii) ≥1 g/g urine total protein to creatinine ratio (proteinuria);
(iii) active urinary sediment;
(iv) positive anti-dsDNA and/or antinuclear antibodies (ANA), optionally wherein positive anti-dsDNA is a titer of ≥30 IU/mL and positive ANA is a titer of ≥1:80;
(v) stable standard treatment regimen characterized by the stable use of a standard therapy for treating the SLE, optionally wherein the stable use is stable use of the standard therapy for at least 30 days; and
(vi) received stable background immunosuppression, optionally wherein the stable background immunosuppression is a stable dose of MMF of ≥1 g/day, with or without corticosteroids, for at least 8 weeks prior to the time of screening or the time of administering the TACI-Fc fusion protein.

48. The method of any of claims 36-41, wherein the Glomerulonephritis is primary Membranous Nephropathy.

49. The method of any of claims 36-41 and 48, wherein the Glomerulonephritis is primary Membranous Nephropathy (pMN) and the subject is selected for treatment if at the time of screening the subject is characterized by one or more of the following:
(i) the subject was diagnosed with pMN≤5 years prior to the screening;
(ii) ≥3.5 g/g urine total protein to creatinine ratio (proteinuria); and
(iii) positive anti-PLA2R1 and/or positive anti-THSD7A antibodies.

50. The method of any of claims 36-49, wherein the subject is selected for treatment if at the time of screening or the time of administering the TACI-Fc fusion protein the subject has received therapy with an Angiotensin-converting enzyme (ACE) inhibitor and/or angiotensin II receptor blocker (ARB), optionally wherein the subject has received a maximally recommended dose of the ACE inhibitor or ARB therapy.

51. The method of any of claims 36-50, wherein the subject is selected for treatment if at the time of screening or at the time of administering the TACI-Fc fusion protein the subject has a stable blood pressure.

52. The method of any of claims 1-9, wherein the autoantibody-related disease or disorder is a hematological disease or disorder.

53. The method of any of claims 1-9 and 52, wherein the autoantibody-related disease or disorder is an autoimmune cytopenia.

54. A method of treating an autoimmune cytopenia, the method comprising:
a) selecting a subject for administration of a TACI-Fc fusion protein that has been diagnosed with an autoimmune cytopenia; and
b) administering to the selected subject the TACI-Fc fusion protein, wherein:
the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
the TACI-Fc fusion protein is administered subcutaneously at a dose of from at or about 80 mg to at or about 480 mg once every four weeks.

55. The method of claim 54, wherein the dose is from at or about 80 mg to at or about 240 mg Q4W.

56. The method of claim 54 or claim 55, wherein the dose is at or about 80 mg Q4W.

57. The method of claim 54 or claim 55, wherein the dose is at or about 240 mg Q4W.

58. The method of any of claims 53-57, wherein the subject is selected for treatment if at the time of screening the subject has active cytopenia.

59. The method of any of claims 53-58, wherein the autoimmune cytopenia is selected from the group consisting of Immune Thrombocytopenia (ITP) and Autoimmune Hemolytic Anemia (AIHA).

60. The method of any of claims 53-59, wherein the autoimmune cytopenia is ITP and the subject is selected for treatment if at the time of screening the subject is characterized by one or more of the following:
(i) the subject was diagnosed with ITP≥3 months prior to the screening;
(ii) platelet count <30,000/μL; and
(iii) received ≥2 prior treatments for treating the ITP, optionally ≥4 prior treatments for treating the ITP.

61. The method of any of claims 53-59, wherein the autoimmune cytopenia is an AIHA and the AIHA is warm AIHA (wAIHA) or cold AIHA (cold agglutinin disease, CAD).

62. The method of any of claims 53-59 and 61, wherein the autoimmune cytopenia is wAIHA or CAD and the subject is selected for treatment if at the time of screening the subject is characterized by one or more of the following:
(i) the subject was diagnosed with wAIHA or CAD≥3 months prior to the screening;
(ii) hemoglobin (Hb)<9 g/dL; and
(iii) received ≥1 prior treatment for treating the AIHA, optionally ≥2 prior treatments for treating the AIHA.

63. The method of claim 62, wherein the autoimmune cytopenia is wAIHA.

64. The method of claim 62, wherein the autoimmune cytopenia is CAD.

65. The method of claim 53-64, wherein the subject is selected for treatment if at the time of screening or the time of administering the TACI-Fc fusion protein, the subject is receiving a stable immunosuppression, optionally wherein the TACI-Fc fusion protein is administered to the subject in combination with concurrent administration of the stable immunosuppression.

66. The method of claim 65, wherein:
the stable immunosuppression comprises a stable dose of a steroid, optionally a corticosteroid, for at least two weeks prior to the time of screening or the time of administering the TACI-Fc fusion protein; and/or
the stable immunosuppression comprises a stable dose of azathioprine, MMF, or a calcineurin inhibitor, optionally cyclosporine, for at least four weeks prior to the time of screening or the time of administering the TACI-Fc fusion protein.

67. The method of any of claims 53-66, wherein the subject is not characterized by having a secondary cytopenia (e.g. systemic autoimmune disease or malignancy) or Evans syndrome.

68. The method of any of claims 53-66, wherein the subject is characterized by having a secondary cytopenia (e.g. systemic autoimmune disease or malignancy) or Evans syndrome.

69. The method of any of claims 1-9, wherein the autoantibody-related disease or disorder is a dermatologic disease or disorder.

70. The method of any of claims 1-9 and 69, wherein the autoantibody-related disease or disorder is an autoimmune bullous dermatosis.

71. A method of treating an autoimmune bullous (blistering) dermatosis, the method comprising:
a) selecting a subject for administration of a TACI-Fc fusion protein that has been diagnosed with an autoimmune bullous (blistering) dermatosis; and
b) administering to the selected subject the TACI-Fc fusion protein, wherein:
the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
the TACI-Fc fusion protein is administered subcutaneously at a dose of from at or about 80 mg to at or about 480 mg once every four weeks.

72. The method of claim 71, wherein the dose is from at or about 80 mg to at or about 240 mg Q4W.

73. The method of claim 71 or claim 72, wherein the dose is at or about 80 mg Q4W.

74. The method of claim 71 or claim 72, wherein the dose is at or about 240 mg Q4W.

75. The method of any of claims 70-74, wherein the subject is selected for treatment if at the time of screening the subject has active blistering disease.

76. The method of any of claims 70-75, wherein the autoimmune bullous (blistering) dermatosis is selected from the group consisting of Pemphigus vulgaris, Pemphigus foliaceus or Bullous Pemphigoid.

77. The method of any of claims 70-76, wherein the autoimmune bullous (blistering) dermatosis is Pemphigus vulgaris or Pemphigus foliaceus and the subject is selected for treatment if at the time of screening the subject is characterized by one or both of the following:
(i) a Pemphigus Disease Area Index (PDAI)≥15; and
(ii) positive anti-Dsg1 or positive anti-Dsg3 antibodies.

78. The method of claim 77, wherein the autoimmune bullous (blistering) dermatosis is Pemphigus vulgaris.

79. The method of claim 78, wherein the autoimmune bullous (blistering) dermatosis is Pemphigus foliaceus.

80. The method of any of claims 70-77, wherein the autoimmune bullous (blistering) dermatosis is Pemphigoid and the subject is selected for treatment if at the time of screening the subject is characterized by one or both of the following:
(i) IgA antibodies; and
(ii) positive anti-Bp180 or positive anti-Bp230 antibodies.

81. The method of claim 70-80, wherein the subject is selected for treatment if at the time of screening or the time of administering the TACI-Fc fusion protein, the subject is receiving a stable immunosuppression.

82. The method of claim 81, wherein the TACI-Fc fusion protein is administered to the subject in combination with concurrent administration of the stable immunosuppression.

83. The method of claim 81 or claim 82, wherein:
the stable immunosuppression comprises a stable dose of a steroid, optionally a corticosteroid, for at least two weeks prior to the time of screening or the time of administering the TACI-Fc fusion protein; and/or
the stable immunosuppression comprises a stable dose of azathioprine, MMF, or a calcineurin inhibitor, optionally cyclosporine, for at least four weeks prior to the time of screening or the time of administering the TACI-Fc fusion protein.

84. The method of any of claims 70-83, wherein the subject is not characterized by having a secondary disease (e.g. paraneoplastic).

85. The method of any of claims 1-9, wherein the autoantibody-related disease or disorder is a neurologic disease or disorder.

86. The method of any of claims 1-9 and 85, wherein the autoantibody-related disease or disorder is Encephalitis.

87. A method of treating Encephalitis, the method comprising:
a) selecting a subject for administration of a TACI-Fc fusion protein that has been diagnosed with Encephalitis; and
b) administering to the selected subject the TACI-Fc fusion protein, wherein:
the TACI-Fc fusion protein is a homodimer of two polypeptides of the formula TACI-linker-Fc, wherein TACI is a variant TACI polypeptide comprising the amino acid substitutions K77E, F78Y and Y102D in the amino acid sequence set forth in SEQ ID NO:13; and
the TACI-Fc fusion protein is administered subcutaneously at a dose of from at or about 80 mg to at or about 480 mg once every four weeks.

88. The method of claim 87, wherein the dose is from at or about 80 mg to at or about 240 mg Q4W.

89. The method of claim 87 or claim 88, wherein the dose is at or about 80 mg Q4W.

90. The method of claim 87 or claim 88, wherein the dose is at or about 240 mg Q4W.

91. The method of any of claims 86-90, wherein the Encephalitis is autoimmune encephalitis.

92. The method of any of claims 86-91, wherein the Encephalitis is Limbic encephalitis.

93. The method of any of claims 1-92, wherein the TACI-Fc fusion protein is administered to the subject Q4W for between 12 weeks and 72 weeks.

94. The method of any of claims 1-93, wherein the TACI-Fc fusion protein is administered to the subject Q4W for 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks or more.

95. The method of any of claims 1-94, wherein the TACI-Fc fusion protein is administered to the subject Q4W for 12 weeks.

96. The method of any of claims 1-94, wherein the TACI-Fc fusion protein is administered to the subject Q4W for 16 weeks.

97. The method of any of claims 1-94, wherein the TACI-Fc fusion protein is administered to the subject Q4W for 24 weeks.

98. The method of any of claims 1-94, wherein the TACI-Fc fusion protein is administered to the subject Q4W for 48 weeks.

99. The method of any of claims 1-98, wherein the variant TACI polypeptide is set forth in SEQ ID NO:26.

100. The method of any of claims 1-98, wherein the linker is a GS linker of between 5 and 20 amino acids in length.

101. The method of any of claims 1-100, wherein the linker is selected from GSGGS (SEQ ID NO: 76), GGGGS (G4S; SEQ ID NO: 77), GSGGGGS (SEQ ID NO: 74), GGGGSGGGGS (2×GGGGS; SEQ ID NO: 78), GGGGSGGGGSGGGGS (3×GGGGS; SEQ ID NO: 79), GGGGSGGGGSGGGGSGGGGS (4×GGGGS, SEQ ID NO:84), GGGGSGGGGSGGGGSGGGGSGGGGS (5×GGGGS, SEQ ID NO: 91), GGGGSSA (SEQ ID NO: 80), or GSGGGGSGGGGS (SEQ ID NO:194) or combinations thereof.

102. The method of any of claims 1-101, wherein the linker is set forth in SEQ ID NO: 74.

103. The method of any of claims 1-102, wherein the Fc is an IgG1 Fc domain.

104. The method of any of claims 1-103, wherein the Fc is a variant IgG1 Fc that exhibits reduced binding affinity to an Fc receptor and/or reduced effector function as compared to a wild-type IgG1 Fc domain.

105. The method of claim 104, wherein the variant IgG1 Fc domain comprises one or more amino acid substitutions selected from L234A, L234V, L235A, L235E, G237A, S267K, R292C, N297G, and V302C, by EU numbering.

106. The method of claim 104 or claim 105, wherein the variant IgG1 Fc comprises the amino acid substitutions L234A, L235E, and G237A by EU numbering.

107. The method of any of claims 103-106, wherein the Fc comprises the amino acid substitution C220S, wherein the residues are numbered according to the EU index of Kabat.

108. The method of any of claims 103-107, wherein the Fc lacks the hinge sequence EPKSS or EPKSC.

109. The method of any of claims 103-108, wherein the Fc region comprises K447del, wherein the residue is numbered according to the EU index of Kabat.

110. The method of claim 1-107 and 109, wherein the Fc comprises the amino acid sequence set forth in SEQ ID NO:73.

111. The method of any of claims 1-107, 109 and 110, wherein the TACI-Fc fusion protein is set forth in SEQ ID NO: 167.

112. The method of claim 1-103, 107-110, wherein the Fc comprises the amino acid sequence set forth in SEQ ID NO:81.

113. The method of any of claims 1-103, 107-110, and 111, wherein the TACI-Fc fusion protein is set forth in SEQ ID NO: 168.

114. The method of any of claims 1-113, wherein the TACI-Fc fusion protein is provided in a formulation comprising an acetic acid buffer having a pH of from about 4.0 to about 6.0, proline at a concentration of from at or about 1% to about 10%, and a surfactant at a concentration of from about 0.005 to about 0.05% (w/v).

115. The method of claim 114, wherein the formulation has a pH of about 5.2.

116. The method of claim 114 or claim 115, wherein the acetic acid buffer comprises a concentration of acetate of from at or about 5 mM to at or about 15 mM.

117. The method of any of claims 114-116, wherein the acetic acid buffer comprises a concentration of acetate of at or about 10 mM.

118. The method of any of claims 114-117, wherein the proline is at a concentration of about 2% to about 5%.

119. The method of any of claims 114-117, wherein the proline is at a concentration of at or about 3%.

120. The method of any of claims 114-119, wherein the surfactant is at a concentration of from about 0.01 to about 0.025% (w/v), optionally at or about 0.015% (w/v).

121. The method of any of claims 114-120, wherein the surfactant is polysorbate 80.

122. The method of any of claims 114-121, wherein the amount of TACI-Fc fusion protein in the formulation is from about 50 mg to about 100 mg.

123. The method of any of claims 114-122, wherein the amount of TACI-Fc fusion protein in the formulation is at or about 80 mg.

124. The method of any of claims 114-123, wherein the concentration of the TACI-Fc fusion protein is between about 50 mg/mL and about 200 mg/mL.

125. The method of any of claims 114-120, wherein the concentration of the TACI-Fc fusion protein is at or about 100 mg/mL.

126. The method of any of claim 1-125, wherein a B cell immune response or activity is reduced in the subject.

127. The method of any of claim 1-126, wherein the numbers of mature and total circulating B cells is reduced in the subject.

128. The method of any of claims 1-127, wherein circulating serum immunoglobulins are reduced in the subject.

129. The method of any of claims 1-128, wherein one or more of B cell maturation, differentiation, and/or proliferation is reduced or inhibited.

130. The method of any of claims 1-129, wherein circulating levels of an APRIL or BAFF protein are reduced in the subject, optionally wherein the APRIL or BAFF protein is a APRIL homotrimer, BAFF homotrimer, APRIL/BAFF heterotrimer, or BAFF 60mer.

131. The method of any of claims 1-130, wherein the subject is a human.

132. The method of claim 131, wherein the subject is an adult subject, optionally 18 years of age or older, optionally 18-65 years of age.


SEQUENCE LISTING (241 sequences)

SEQ ID NO: 1 - protein, 82 aa, synthetic construct
  REGION 1..82: note = TACI WT ECD
VAMRSCPEEQYWDPLLGTCMSCKTICNHQSQRTCAAFCRSLSCRKEQGKFYDHLLRDCISCASICGQHPKQCAYFCENKLRS

SEQ ID NO: 2 - protein, 82 aa, synthetic construct
  REGION 1..82: note = TACI L82P ECD
VAMRSCPEEQYWDPLLGTCMSCKTICNHQSQRTCAAFCRSLSCRKEQGKFYDHPLRDCISCASICGQHPKQCAYFCENKLRS

SEQ ID NO: 3 - protein, 82 aa, synthetic construct
  REGION 1..82: note = TACI D85E, K98T ECD
VAMRSCPEEQYWDPLLGTCMSCKTICNHQSQRTCAAFCRSLSCRKEQGKFYDHLLRECISCASICGQHPTQCAYFCENKLRS

SEQ ID NO: 4 - protein, 82 aa, synthetic construct
  REGION 1..82: note = TACI I87L, K98T ECD
VAMRSCPEEQYWDPLLGTCMSCKTICNHQSQRTCAAFCRSLSCRKEQGKFYDHLLRDCLSCASICGQHPTQCAYFCENKLRS

SEQ ID NO: 5 - protein, 82 aa, synthetic construct
  REGION 1..82: note = TACI R60G, Q75E, L82P ECD
VAMRSCPEEQYWDPLLGTCMSCKTICNHQSQGTCAAFCRSLSCRKEEGKFYDHPLRDCISCASICGQHPKQCAYFCENKLRS

SEQ ID NO: 6 - protein, 82 aa, synthetic construct
  REGION 1..82: note = TACI R60G, C86Y ECD
VAMRSCPEEQYWDPLLGTCMSCKTICNHQSQGTCAAFCRSLSCRKEQGKFYDHLLRDYISCASICGQHPKQCAYFCENKLRS

SEQ ID NO: 7 - protein, 82 aa, synthetic construct
  REGION 1..82: note = TACI A101D ECD
VAMRSCPEEQYWDPLLGTCMSCKTICNHQSQRTCAAFCRSLSCRKEQGKFYDHLLRDCISCASICGQHPKQCDYFCENKLRS

SEQ ID NO: 8 - protein, 82 aa, synthetic construct
  REGION 1..82: note = TACI C86Y ECD
VAMRSCPEEQYWDPLLGTCMSCKTICNHQSQRTCAAFCRSLSCRKEQGKFYDHLLRDYISCASICGQHPKQCAYFCENKLRS

SEQ ID NO: 9 - protein, 82 aa, synthetic construct
  REGION 1..82: note = TACI W40R, L82P, F103Y ECD
VAMRSCPEEQYRDPLLGTCMSCKTICNHQSQRTCAAFCRSLSCRKEQGKFYDHPLRDCISCASICGQHPKQCAYYCENKLRS

SEQ ID NO: 10 - protein, 82 aa, synthetic construct
  REGION 1..82: note = TACI W40R, Q59R, T61P, K98T ECD
VAMRSCPEEQYRDPLLGTCMSCKTICNHQSRRPCAAFCRSLSCRKEQGKFYDHLLRDCISCASICGQHPTQCAYFCENKLRS

SEQ ID NO: 11 - protein, 82 aa, synthetic construct
  REGION 1..82: note = TACI L82P, I87L ECD
VAMRSCPEEQYWDPLLGTCMSCKTICNHQSQRTCAAFCRSLSCRKEQGKFYDHPLRDCLSCASICGQHPKQCAYFCENKLRS

SEQ ID NO: 12 - protein, 82 aa, synthetic construct
  REGION 1..82: note = TACI G76S, P97S ECD
VAMRSCPEEQYWDPLLGTCMSCKTICNHQSQRTCAAFCRSLSCRKEQSKFYDHLLRDCISCASICGQHSKQCAYFCENKLRS

SEQ ID NO: 13 - protein, 43 aa, synthetic construct
  REGION 1..43: note = TACI WT ECD
SLSCRKEQGKFYDHLLRDCISCASICGQHPKQCAYFCENKLRS

SEQ ID NO: 14 - protein, 43 aa, synthetic construct
  REGION 1..43: note = TACI D85V ECD
SLSCRKEQGKFYDHLLRVCISCASICGQHPKQCAYFCENKLRS

SEQ ID NO: 15 - protein, 43 aa, synthetic construct
  REGION 1..43: note = TACI E74V ECD
SLSCRKVQGKFYDHLLRDCISCASICGQHPKQCAYFCENKLRS

SEQ ID NO: 16 - protein, 43 aa, synthetic construct
  REGION 1..43: note = TACI R84L ECD
SLSCRKEQGKFYDHLLLDCISCASICGQHPKQCAYFCENKLRS

SEQ ID NO: 17 - protein, 43 aa, synthetic construct
  REGION 1..43: note = TACI K77E, R84L, F103Y ECD
SLSCRKEQGEFYDHLLLDCISCASICGQHPKQCAYYCENKLRS

SEQ ID NO: 18 - protein, 43 aa, synthetic construct
  REGION 1..43: note = TACI Y79F, Q99E ECD
SLSCRKEQGKFFDHLLRDCISCASICGQHPKECAYFCENKLRS

SEQ ID NO: 19 - protein, 43 aa, synthetic construct
  REGION 1..43: note = TACI Y79F ECD
SLSCRKEQGKFFDHLLRDCISCASICGQHPKQCAYFCENKLRS

SEQ ID NO: 20 - protein, 43 aa, synthetic construct
  REGION 1..43: note = TACI R84G ECD
SLSCRKEQGKFYDHLLGDCISCASICGQHPKQCAYFCENKLRS

SEQ ID NO: 21 - protein, 82 aa, synthetic construct
  REGION 1..82: note = TACI L83S, F103S ECD
VAMRSCPEEQYWDPLLGTCMSCKTICNHQSQRTCAAFCRSLSCRKEQGKFYDHLSRDCISCASICGQHPKQCAYSCENKLRS

SEQ ID NO: 22 - protein, 82 aa, synthetic construct
  REGION 1..82: note = TACI L82H ECD
VAMRSCPEEQYWDPLLGTCMSCKTICNHQSQRTCAAFCRSLSCRKEQGKFYDHHLRDCISCASICGQHPKQCAYFCENKLRS

SEQ ID NO: 23 - protein, 43 aa, synthetic construct
  REGION 1..43: note = TACI A101D ECD
SLSCRKEQGKFYDHLLRDCISCASICGQHPKQCDYFCENKLRS

SEQ ID NO: 24 - protein, 43 aa, synthetic construct
  REGION 1..43: note = TACI K77E, R84Q ECD
SLSCRKEQGEFYDHLLQDCISCASICGQHPKQCAYFCENKLRS

SEQ ID NO: 25 - protein, 43 aa, synthetic construct
  REGION 1..43: note = TACI K77E, A101D ECD
SLSCRKEQGEFYDHLLRDCISCASICGQHPKQCDYFCENKLRS

SEQ ID NO: 26 - protein, 43 aa, synthetic construct
  REGION 1..43: note = TACI K77E, F78Y, Y102D ECD
SLSCRKEQGEYYDHLLRDCISCASICGQHPKQCADFCENKLRS

SEQ ID NO: 27 - protein, 43 aa, synthetic construct
  REGION 1..43: note = TACI Q75E, R84Q ECD
SLSCRKEEGKFYDHLLQDCISCASICGQHPKQCAYFCENKLRS

SEQ ID NO: 28 - protein, 43 aa, synthetic construct
  REGION 1..43: note = TACI Q75R, R84G, I92V ECD
SLSCRKERGKFYDHLLGDCISCASVCGQHPKQCAYFCENKLRS

SEQ ID NO: 29 - protein, 43 aa, synthetic construct
  REGION 1..43: note = TACI K77E, A101D, Y102D ECD
SLSCRKEQGEFYDHLLRDCISCASICGQHPKQCDDFCENKLRS

SEQ ID NO: 30 - protein, 43 aa, synthetic construct
  REGION 1..43: note = TACI R84Q ECD
SLSCRKEQGKFYDHLLQDCISCASICGQHPKQCAYFCENKLRS

SEQ ID NO: 31 - protein, 43 aa, synthetic construct
  REGION 1..43: note = TACI R84Q, S88N, A101D ECD
SLSCRKEQGKFYDHLLQDCINCASICGQHPKQCDYFCENKLRS

SEQ ID NO: 32 - protein, 43 aa, synthetic construct
  REGION 1..43: note = TACI K77E ECD
SLSCRKEQGEFYDHLLRDCISCASICGQHPKQCAYFCENKLRS

SEQ ID NO: 33 - protein, 43 aa, synthetic construct
  REGION 1..43: note = TACI R84Q, F103V ECD
SLSCRKEQGKFYDHLLQDCISCASICGQHPKQCAYVCENKLRS

SEQ ID NO: 34 - protein, 43 aa, synthetic construct
  REGION 1..43: note = TACI K77E, Q95R, A101D ECD
SLSCRKEQGEFYDHLLRDCISCASICGRHPKQCDYFCENKLRS

SEQ ID NO: 35 - protein, 43 aa, synthetic construct
  REGION 1..43: note = TACI I87M, A101D ECD
SLSCRKEQGKFYDHLLRDCMSCASICGQHPKQCDYFCENKLRS

SEQ ID NO: 36 - DNA, 246 bp, synthetic construct
  misc_feature 1..246: note = TACI WT ECD
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgcttatttctgtgaaaataagcttcgatct

SEQ ID NO: 37 - DNA, 246 bp, synthetic construct
  misc_feature 1..246: note = TACI L82P ECD
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcctttgcgagactgtataagctgtgcgtctatttgtggacaacaccctaaacaatgtgcttatttctgtgaaaataagcttcgatct

SEQ ID NO: 38 - DNA, 246 bp, synthetic construct
  misc_feature 1..246: note = TACI D85E, K98T ECD
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagaatgtataagctgtgcgtctatttgtggacaacaccctacacaatgtgcttatttctgtgaaaataagcttcgatct

SEQ ID NO: 39 - DNA, 246 bp, synthetic construct
  misc_feature 1..246: note = TACI I87L, K98T ECD
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtttaagctgtgcgtctatttgtggacaacaccctacacaatgtgcttatttctgtgaaaataagcttcgatct

SEQ ID NO: 40 - DNA, 246 bp, synthetic construct
  misc_feature 1..246: note = TACI R60G, Q75E, L82P ECD
  source 1..246: mol_type = other DNA
gtggcaatgcgctcatgcccagaggaacaatattgggatccgcttcttgggacgtgtatgagctgcaagaccatctgtaatcatcaatcccaagggacatgcgcagctttctgcaggagtctctcttgcaggaaagaggaaggcaaattttacgaccatcctttgcgagactgtataagctgtgcgtctatttgtggacaacaccctaaacaatgtgcttatttctgtgaaaataagcttcgatct

SEQ ID NO: 41 - DNA, 246 bp, synthetic construct
  misc_feature 1..246: note = TACI R60G, C86Y ECD
  source 1..246: mol_type = other DNA
gtggcaatgcgctcatgcccagaggaacaatattgggatccgcttcttgggacgtgtatgagctgcaagaccatctgtaatcatcaatcccaagggacatgcgcagctttctgcaggagtctctcttgcaggaaagagcaaggcaaattttacgaccatcttttgcgagactacataagctgtgcgtctatttgtggacaacaccctaaacaatgtgcttatttctgtgaaaataagcttcgatct

SEQ ID NO: 42 - DNA, 246 bp, synthetic construct
  misc_feature 1..246: note = TACI A101D ECD
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgattatttctgtgaaaataagcttcgatct

SEQ ID NO: 43 - DNA, 246 bp, synthetic construct
  misc_feature 1..246: note = TACI C86Y ECD
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactatataagctgtgcgtctatttgtggacaacaccctaaacaatgtgcttatttctgtgaaaataagcttcgatct

SEQ ID NO: 44 - DNA, 246 bp, synthetic construct
  misc_feature 1..246: note = TACI W40R, L82P, F103Y ECD
  source 1..246: mol_type = other DNA
gtggcaatgcgctcatgcccagaggaacaatatcgggatccgcttcttgggacgtgtatgagctgcaagaccatctgtaatcatcaatcccaaaggacatgcgcagctttctgcaggagtctctcttgcaggaaagagcaaggcaaattttacgaccatcctttgcgagactgtataagctgtgcgtctatttgtggtcaacaccctaaacaatgtgcttattactgtgaaaataagcttcgatct

SEQ ID NO: 45 - DNA, 246 bp, synthetic construct
  misc_feature 1..246: note = TACI W40R, Q59R, T61P, K98T ECD
  source 1..246: mol_type = other DNA
gtggcaatgcgctcatgcccagaggaacaatatcgggatccgcttcttgggacgtgtatgagctgcaagaccatctgtaatcatcaatcccgaaggccatgcgcagcattctgcaggagtctctcttgcaggaaagagcaaggcaaattttacgaccatcttttgcgagactgtataagctgtgcgtctatttgtggacaacaccctacacaatgtgcttatttctgtgaaaataagcttcgatct

SEQ ID NO: 46 - DNA, 246 bp, synthetic construct
  misc_feature 1..246: note = TACI L82P, I87L ECD
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcctttgcgagactgtttaagctgtgcgtctatttgtggacaacaccctaaacaatgtgcttatttctgtgaaaataagcttcgatct

SEQ ID NO: 47 - DNA, 246 bp, synthetic construct
  misc_feature 1..246: note = TACI G76S, P97S ECD
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaagcaaattttacgaccatcttttgcgagactgtataagctgtgcgtctatttgtggtcaacactctaaacaatgtgcttatttctgtgaaaataagcttcgatct

SEQ ID NO: 48 - DNA, 129 bp, synthetic construct
  misc_feature 1..129: note = TACI WT ECD
  source 1..129: mol_type = other DNA
agtctctcttgcaggaaagagcaaggcaaattttacgaccatcttttgcgagactgtataagctgtgcgtctatttgtggacaacaccctaaacaatgtgcttatttctgtgaaaataagcttcgatct

SEQ ID NO: 49 - DNA, 129 bp, synthetic construct
  misc_feature 1..129: note = TACI D85V ECD
  source 1..129: mol_type = other DNA
agtctctcttgcaggaaagagcaaggcaaattttacgaccatcttttgcgagtctgtataagctgtgcgtctatttgtggacaacaccctaaacaatgtgcttatttctgtgaaaataagcttcgatct

SEQ ID NO: 50 - DNA, 129 bp, synthetic construct
  misc_feature 1..129: note = TACI E74V ECD
  source 1..129: mol_type = other DNA
agtctctcttgcaggaaagtgcaaggcaaattttacgaccatcttttgcgagactgtataagctgtgcgtctatttgtggacaacaccctaaacaatgtgcttatttctgtgaaaataagcttcgatct

SEQ ID NO: 51 - DNA, 129 bp, synthetic construct
  misc_feature 1..129: note = TACI R84L ECD
  source 1..129: mol_type = other DNA
agtctctcttgcaggaaagagcaaggcaaattttacgaccatcttttgctagactgtataagctgtgcgtctatttgtggacaacaccctaaacaatgtgcttatttctgtgaaaataagcttcgatct

SEQ ID NO: 52 - DNA, 129 bp, synthetic construct
  misc_feature 1..129: note = TACI K77E, R84L, F103Y ECD
  source 1..129: mol_type = other DNA
agtctctcttgcaggaaagagcaaggcgaattttacgaccatcttttgctagactgtataagctgtgcgtctatttgtggacaacaccctaaacaatgtgcttattactgtgaaaataagcttcgatct

SEQ ID NO: 53 - DNA, 129 bp, synthetic construct
  misc_feature 1..129: note = TACI Y79F, Q99E ECD
  source 1..129: mol_type = other DNA
agtctctcttgcaggaaagagcaaggcaaatttttcgaccatcttttgcgagactgtataagctgtgcgtctatttgtggacaacaccctaaagaatgtgcttatttctgtgaaaataagcttcgatct

SEQ ID NO: 54 - DNA, 129 bp, synthetic construct
  misc_feature 1..129: note = TACI Y79F ECD
  source 1..129: mol_type = other DNA
agtctctcttgcaggaaagagcaaggcaaatttttcgaccatcttttgcgagactgtataagctgtgcgtctatttgtggacaacaccctaaacaatgtgcttatttctgtgaaaataagcttcgatct

SEQ ID NO: 55 - DNA, 129 bp, synthetic construct
  misc_feature 1..129: note = TACI R84G ECD
  source 1..129: mol_type = other DNA
agtctctcttgcaggaaagagcaaggcaaattttacgaccatcttttgggagactgtataagctgtgcgtctatttgtggacaacaccctaaacaatgtgcttatttctgtgaaaataagcttcgatct

SEQ ID NO: 56 - DNA, 246 bp, synthetic construct
  misc_feature 1..246: note = TACI L83S, F103S ECD
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatctttcgcgagactgtataagctgtgcgtctatttgtggacaacaccctaaacaatgtgcttattcctgtgaaaataagcttcgatct

SEQ ID NO: 57 - DNA, 246 bp, synthetic construct
  misc_feature 1..246: note = TACI L82H ECD
  source 1..246: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcatttgcgagactgtataagctgtgcgtctatctgtggacaacaccctaaacaatgtgcttatttctgtgaaaataagcttcgatct

SEQ ID NO: 58 - DNA, 129 bp, synthetic construct
  misc_feature 1..129: note = TACI A101D ECD
  source 1..129: mol_type = other DNA
agtctctcttgcaggaaagagcaaggcaaattttacgaccatcttttgcgagactgtataagctgtgcgtctatttgtggacaacaccctaaacaatgtgattatttctgtgaaaataagcttcgatct

SEQ ID NO: 59 - DNA, 129 bp, synthetic construct
  misc_feature 1..129: note = TACI K77E, R84Q ECD
  source 1..129: mol_type = other DNA
agtctctcttgcaggaaagagcaaggcgaattttacgaccatcttttgcaagactgtataagctgtgcgtctatttgtggacaacaccctaaacaatgtgcttatttctgtgaaaataagcttcgatct

SEQ ID NO: 60 - DNA, 129 bp, synthetic construct
  misc_feature 1..129: note = TACI K77E, A101D ECD
  source 1..129: mol_type = other DNA
agtctctcttgcaggaaagagcaaggcgaattttacgaccatcttttgcgagactgtataagctgtgcgtctatttgtggacaacaccctaaacaatgtgattatttctgtgaaaataagcttcgatct

SEQ ID NO: 61 - DNA, 129 bp, synthetic construct
  misc_feature 1..129: note = TACI K77E, F78Y, Y102D ECD
  source 1..129: mol_type = other DNA
agtctctcttgcaggaaagagcaaggcgaatattacgaccatcttttgcgagactgtataagctgtgcgtctatttgtggacaacaccctaaacaatgtgctgatttctgtgaaaataagcttcgatct

SEQ ID NO: 62 - DNA, 129 bp, synthetic construct
  misc_feature 1..129: note = TACI Q75E, R84Q ECD
  source 1..129: mol_type = other DNA
agtctctcttgcaggaaagaggaaggcaaattttacgaccatcttttgcaagactgtataagctgtgcgtctatttgtggacaacaccctaaacaatgtgcttatttctgtgaaaataagcttcgatct

SEQ ID NO: 63 - DNA, 129 bp, synthetic construct
  misc_feature 1..129: note = TACI Q75R, R84G, I92V ECD
  source 1..129: mol_type = other DNA
agtctctcttgcaggaaagagcgaggcaaattttacgaccatcttttgggagactgtataagctgtgcgtctgtttgtggacaacaccctaaacaatgtgcttatttctgtgaaaataagcttcgatct

SEQ ID NO: 64 - DNA, 129 bp, synthetic construct
  misc_feature 1..129: note = TACI K77E, A101D, Y102D ECD
  source 1..129: mol_type = other DNA
agtctctcttgcaggaaagagcaaggcgaattttacgaccatcttttgcgagactgtataagctgtgcgtctatttgtggacaacaccctaaacaatgtgatgatttctgtgaaaataagcttcgatct

SEQ ID NO: 65 - DNA, 129 bp, synthetic construct
  misc_feature 1..129: note = TACI R84Q ECD
  source 1..129: mol_type = other DNA
agtctctcttgcaggaaagagcaaggcaaattttatgaccatcttttgcaagactgtataagctgtgcgtctatttgtggacaacaccctaaacaatgtgcttatttctgtgaaaataagcttcgatct

SEQ ID NO: 66 - DNA, 129 bp, synthetic construct
  misc_feature 1..129: note = TACI R84Q, S88N, A101D ECD
  source 1..129: mol_type = other DNA
agtctctcttgcaggaaagagcaaggcaaattttacgaccatcttttgcaagactgtataaactgtgcgtctatatgtggacaacaccctaaacaatgtgattatttctgtgaaaataagcttcgatct

SEQ ID NO: 67 - DNA, 129 bp, synthetic construct
  misc_feature 1..129: note = TACI K77E ECD
  source 1..129: mol_type = other DNA
agtctctcttgcaggaaagagcaaggcgaattttacgaccatcttttgcgagactgtataagctgtgcgtctatttgtggacaacaccctaaacaatgtgcttatttctgtgaaaataagcttcgatct

SEQ ID NO: 68 - DNA, 129 bp, synthetic construct
  misc_feature 1..129: note = TACI R84Q, F103V ECD
  source 1..129: mol_type = other DNA
agtctctcttgcaggaaagagcaaggcaaattttacgaccatcttttgcaagactgtataagctgtgcgtctatttgtggacaacaccctaaacaatgtgcttatgtctgtgaaaataagcttcgatct

SEQ ID NO: 69 - DNA, 129 bp, synthetic construct
  misc_feature 1..129: note = TACI K77E, Q95R, A101D ECD
  source 1..129: mol_type = other DNA
agtctctcttgcaggaaagagcaaggcgaattttacgaccatcttttgcgagactgtataagctgtgcgtctatttgtggacgacaccctaaacaatgtgattatttctgtgaaaataagcttcgatct

SEQ ID NO: 70 - DNA, 129 bp, synthetic construct
  misc_feature 1..129: note = TACI I87M, A101D ECD
  source 1..129: mol_type = other DNA
agtctctcttgcaggaaagagcaaggcaaattttacgaccatcttttgcgagactgtatgagctgtgcgtcaatttgtggacaacaccctaaacaatgtgattatttctgtgaaaataagcttcgatct

SEQ ID NO: 71 - protein, 232 aa, synthetic construct
  REGION 1..232: note = IgG1 Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 8 aa, synthetic construct
  REGION 1..8: note = FLAG-tag
DYKDDDDK

SEQ ID NO: 73 - protein, 231 aa, synthetic construct
  REGION 1..231: note = Fc C220S/L234A/L235E/G237A/K447del
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 7 aa, synthetic construct
  REGION 1..7: note = Linker
GSGGGGS

SEQ ID NO: 75 - protein, 232 aa, synthetic construct
  REGION 1..232: note = Fc C220S/L234A/L235E/G237A/E356D/M358L
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 5 aa, synthetic construct
  REGION 1..5: note = Linker
GSGGS

SEQ ID NO: 77 - protein, 5 aa, synthetic construct
  REGION 1..5: note = LINKER
GGGGS

SEQ ID NO: 78 - protein, 10 aa, synthetic construct
  REGION 1..10: note = LINKER
GGGGSGGGGS

SEQ ID NO: 79 - protein, 15 aa, synthetic construct
  REGION 1..15: note = LINKER
GGGGSGGGGSGGGGS

SEQ ID NO: 80 - protein, 7 aa, synthetic construct
  REGION 1..7: note = LINKER
GGGGSSA

SEQ ID NO: 81 - protein, 231 aa, synthetic construct
  REGION 1..231: note = Fc (E356D and M358L)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 232 aa, synthetic construct
  REGION 1..232: note = Fc (C5S (C220S), R77C, (R292C),N82G (N297G), V87C (V302C))
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 232 aa, synthetic construct
  REGION 1..232: note = Fc with C220S/L234A/L235E/G237A
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 20 aa, synthetic construct
  REGION 1..20: note = LINKER
GGGGSGGGGSGGGGSGGGGS

SEQ ID NO: 85 - protein, 323 aa, synthetic construct
  REGION 1..323: note = Fc (G4S)2 TACI (516)
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSVAMRSCPEEQYWDPLLGTCMSCKTICNHQSQRTCAAFCRSLSCRKEQGKFYDHLLRDCISCASICGQHPKQCAYFCENKLRS

SEQ ID NO: 86 - protein, 294 aa, synthetic construct
  REGION 1..294: note = Fc (G4S)4 TACI (541)
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSLSCRKEQGEYYDHLLRDCISCASICGQHPKQCADFCENKLRS

SEQ ID NO: 87 - protein, 30 aa, synthetic construct
  REGION 1..30: note = LINKER
EAAAKEAAAKEAAAKEAAAKEAAAKEAAAK

SEQ ID NO: 88 - protein, 293 aa, synthetic construct
  REGION 1..293: note = FULL LENGTH TACI
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 89 - protein, 230 aa, synthetic construct
  REGION 1..230: note = Fc K S139C, D141E, L143M, T151W
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 90 - protein, 230 aa, synthetic construct
  REGION 1..230: note = Fc H Y134C, D141E, L143M, T151S, L153A
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 91 - protein, 25 aa, synthetic construct
  REGION 1..25: note = LINKER
GGGGSGGGGSGGGGSGGGGSGGGGS

SEQ ID NO: 92 - protein, 43 aa, synthetic construct
  REGION 1..43: note = TACI L82P ECD
SLSCRKEQGKFYDHPLRDCISCASICGQHPKQCAYFCENKLRS

SEQ ID NO: 93 - protein, 43 aa, synthetic construct
  REGION 1..43: note = TACI D85E, K98T ECD
SLSCRKEQGKFYDHLLRECISCASICGQHPTQCAYFCENKLRS

SEQ ID NO: 94 - protein, 43 aa, synthetic construct
  REGION 1..43: note = TACI I87L, K98T ECD
SLSCRKEQGKFYDHLLRDCLSCASICGQHPTQCAYFCENKLRS

SEQ ID NO: 95 - protein, 43 aa, synthetic construct; identical to SEQ ID NO: 23
  REGION 1..43: note = TACI A101D ECD

SEQ ID NO: 96 - protein, 43 aa, synthetic construct
  REGION 1..43: note = TACI C86Y ECD
SLSCRKEQGKFYDHLLRDYISCASICGQHPKQCAYFCENKLRS

SEQ ID NO: 97 - protein, 43 aa, synthetic construct
  REGION 1..43: note = TACI L82P, I87L ECD
SLSCRKEQGKFYDHPLRDCLSCASICGQHPKQCAYFCENKLRS

SEQ ID NO: 98 - protein, 43 aa, synthetic construct
  REGION 1..43: note = TACI G76S, P97S ECD
SLSCRKEQSKFYDHLLRDCISCASICGQHSKQCAYFCENKLRS

SEQ ID NO: 99 - protein, 43 aa, synthetic construct
  REGION 1..43: note = TACI L83S, F103S ECD
SLSCRKEQGKFYDHLSRDCISCASICGQHPKQCAYSCENKLRS

SEQ ID NO: 100 - protein, 43 aa, synthetic construct
  REGION 1..43: note = TACI L82H ECD
SLSCRKEQGKFYDHHLRDCISCASICGQHPKQCAYFCENKLRS

SEQ ID NO: 101 - protein, 82 aa, synthetic construct
  REGION 1..82: note = TACI D85V ECD
VAMRSCPEEQYWDPLLGTCMSCKTICNHQSQRTCAAFCRSLSCRKEQGKFYDHLLRVCISCASICGQHPKQCAYFCENKLRS

SEQ ID NO: 102 - protein, 82 aa, synthetic construct
  REGION 1..82: note = TACI E74V ECD
VAMRSCPEEQYWDPLLGTCMSCKTICNHQSQRTCAAFCRSLSCRKVQGKFYDHLLRDCISCASICGQHPKQCAYFCENKLRS

SEQ ID NO: 103 - protein, 82 aa, synthetic construct
  REGION 1..82: note = TACI R84L ECD
VAMRSCPEEQYWDPLLGTCMSCKTICNHQSQRTCAAFCRSLSCRKEQGKFYDHLLLDCISCASICGQHPKQCAYFCENKLRS

SEQ ID NO: 104 - protein, 82 aa, synthetic construct
  REGION 1..82: note = TACI K77E, R84L, F103Y ECD
VAMRSCPEEQYWDPLLGTCMSCKTICNHQSQRTCAAFCRSLSCRKEQGEFYDHLLLDCISCASICGQHPKQCAYYCENKLRS

SEQ ID NO: 105 - protein, 82 aa, synthetic construct
  REGION 1..82: note = TACI Y79F, Q99E ECD
VAMRSCPEEQYWDPLLGTCMSCKTICNHQSQRTCAAFCRSLSCRKEQGKFFDHLLRDCISCASICGQHPKECAYFCENKLRS

SEQ ID NO: 106 - protein, 82 aa, synthetic construct
  REGION 1..82: note = TACI Y79F ECD
VAMRSCPEEQYWDPLLGTCMSCKTICNHQSQRTCAAFCRSLSCRKEQGKFFDHLLRDCISCASICGQHPKQCAYFCENKLRS

SEQ ID NO: 107 - protein, 82 aa, synthetic construct
  REGION 1..82: note = TACI R84G ECD
VAMRSCPEEQYWDPLLGTCMSCKTICNHQSQRTCAAFCRSLSCRKEQGKFYDHLLGDCISCASICGQHPKQCAYFCENKLRS

SEQ ID NO: 108 - protein, 82 aa, synthetic construct; identical to SEQ ID NO: 7
  REGION 1..82: note = TACI A101D ECD

SEQ ID NO: 109 - protein, 82 aa, synthetic construct
  REGION 1..82: note = TACI K77E, R84Q ECD
VAMRSCPEEQYWDPLLGTCMSCKTICNHQSQRTCAAFCRSLSCRKEQGEFYDHLLQDCISCASICGQHPKQCAYFCENKLRS

SEQ ID NO: 110 - protein, 82 aa, synthetic construct
  REGION 1..82: note = TACI K77E, A101D ECD
VAMRSCPEEQYWDPLLGTCMSCKTICNHQSQRTCAAFCRSLSCRKEQGEFYDHLLRDCISCASICGQHPKQCDYFCENKLRS

SEQ ID NO: 111 - protein, 82 aa, synthetic construct
  REGION 1..82: note = TACI K77E, F78Y, Y102D ECD
VAMRSCPEEQYWDPLLGTCMSCKTICNHQSQRTCAAFCRSLSCRKEQGEYYDHLLRDCISCASICGQHPKQCADFCENKLRS

SEQ ID NO: 112 - protein, 82 aa, synthetic construct
  REGION 1..82: note = TACI Q75E, R84Q ECD
VAMRSCPEEQYWDPLLGTCMSCKTICNHQSQRTCAAFCRSLSCRKEEGKFYDHLLQDCISCASICGQHPKQCAYFCENKLRS

SEQ ID NO: 113 - protein, 82 aa, synthetic construct
  REGION 1..82: note = TACI Q75R, R84G, I92V ECD
VAMRSCPEEQYWDPLLGTCMSCKTICNHQSQRTCAAFCRSLSCRKERGKFYDHLLGDCISCASVCGQHPKQCAYFCENKLRS

SEQ ID NO: 114 - protein, 82 aa, synthetic construct
  REGION 1..82: note = TACI K77E, A101D, Y102D ECD
VAMRSCPEEQYWDPLLGTCMSCKTICNHQSQRTCAAFCRSLSCRKEQGEFYDHLLRDCISCASICGQHPKQCDDFCENKLRS

SEQ ID NO: 115 - protein, 82 aa, synthetic construct
  REGION 1..82: note = TACI R84Q ECD
VAMRSCPEEQYWDPLLGTCMSCKTICNHQSQRTCAAFCRSLSCRKEQGKFYDHLLQDCISCASICGQHPKQCAYFCENKLRS

SEQ ID NO: 116 - protein, 82 aa, synthetic construct
  REGION 1..82: note = TACI R84Q, S88N, A101D ECD
VAMRSCPEEQYWDPLLGTCMSCKTICNHQSQRTCAAFCRSLSCRKEQGKFYDHLLQDCINCASICGQHPKQCDYFCENKLRS

SEQ ID NO: 117 - protein, 82 aa, synthetic construct
  REGION 1..82: note = TACI K77E ECD
VAMRSCPEEQYWDPLLGTCMSCKTICNHQSQRTCAAFCRSLSCRKEQGEFYDHLLRDCISCASICGQHPKQCAYFCENKLRS

SEQ ID NO: 118 - protein, 82 aa, synthetic construct
  REGION 1..82: note = TACI R84Q, F103V ECD
VAMRSCPEEQYWDPLLGTCMSCKTICNHQSQRTCAAFCRSLSCRKEQGKFYDHLLQDCISCASICGQHPKQCAYVCENKLRS

SEQ ID NO: 119 - protein, 82 aa, synthetic construct
  REGION 1..82: note = TACI K77E, Q95R, A101D ECD
VAMRSCPEEQYWDPLLGTCMSCKTICNHQSQRTCAAFCRSLSCRKEQGEFYDHLLRDCISCASICGRHPKQCDYFCENKLRS

SEQ ID NO: 120 - protein, 82 aa, synthetic construct
  REGION 1..82: note = TACI I87M, A101D ECD
VAMRSCPEEQYWDPLLGTCMSCKTICNHQSQRTCAAFCRSLSCRKEQGKFYDHLLRDCMSCASICGQHPKQCDYFCENKLRS

SEQ ID NO: 121 - protein, 660 aa, synthetic construct
  REGION 1..660: note = CTLA4 113 GSG4S Fc K/ BCMA 357 GSG4S Fc H
KAMHVAQPAVVFASSHGIASFVCEYASPWKATEVRVTVLRQADSQVTEVCAATYMTGNELTFLDDSICTGTSSGNQVNLTIQGLRAMDTGLYICKVEQMYPPPYLLGIGNGTQIYVIDPEPCPDSDGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGLQMAGQCSQNEYFDSLLYACIPCQLRCSSNTPPLTCQRYCNASVTNSVKGTNAGSGGSGGSGGSGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 122 - protein, 166 aa, synthetic construct
  REGION 1..166: note = TACI ECD (1-166)
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYST

SEQ ID NO: 123 - protein, 5 aa, synthetic construct
  REGION 1..5: note = 1xEAAAK
EAAAK

SEQ ID NO: 124 - protein, 10 aa, synthetic construct
  REGION 1..10: note = 2xEAAAK
EAAAKEAAAK

SEQ ID NO: 125 - protein, 15 aa, synthetic construct
  REGION 1..15: note = 3xEAAAK
EAAAKEAAAKEAAAK

SEQ ID NO: 126 - protein, 20 aa, synthetic construct
  REGION 1..20: note = 4xEAAAK
EAAAKEAAAKEAAAKEAAAK

SEQ ID NO: 127 - protein, 25 aa, synthetic construct
  REGION 1..25: note = 5xEAAAK
EAAAKEAAAKEAAAKEAAAKEAAAK

SEQ ID NO: 128 - protein, 232 aa, synthetic construct
  REGION 1..232: note = knob Fc
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 232 aa, synthetic construct
  REGION 1..232: note = hole Fc
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 130 - protein, 81 aa, synthetic construct
  REGION 1..81: note = atacicept (TACI portion, 30-110)
AMRSCPEEQYWDPLLGTCMSCKTICNHQSQRTCAAFCRSLSCRKEQGKFYDHLLRDCISCASICGQHPKQCAYFCENKLRS

SEQ ID NO: 131 - protein, 106 aa, synthetic construct
  REGION 1..106: note = telitacicept (TACI portion, 13-118)
SRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPEL

SEQ ID NO: 132 - protein, 313 aa, synthetic construct
  REGION 1..313: note = atacicept
AMRSCPEEQYWDPLLGTCMSCKTICNHQSQRTCAAFCRSLSCRKEQGKFYDHLLRDCISCASICGQHPKQCAYFCENKLRSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 13 aa, synthetic construct
  REGION 1..13: note = linker
GGGGSGGGGSAAA

SEQ ID NO: 134 - protein, 231 aa, synthetic construct
  REGION 1..231: note = Fc with C220S/E233P/L234V/L235A/G236del/S267K
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 231 aa, synthetic construct
  REGION 1..231: note = Fc (C5S (C220S), R77C, (R292C),N82G (N297G), V87C (V302C),L232del (K447del))
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 231 aa, synthetic construct
  REGION 1..231: note = Fc with C220S/L234A/L235E/G237A/K447del
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 230 aa, synthetic construct
  REGION 1..230: note = Fc with C220S/E233P/L234V/L235A/G236del/S267K/K447del
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 235 aa, synthetic construct
  REGION 1..235: note = IgG2 Fc
TKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 139 - protein, 229 aa, synthetic construct
  REGION 1..229: note = IgG4 Fc
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 229 aa, synthetic construct
  REGION 1..229: note = IgG4 Fc S228P
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 141 - protein, 231 aa, synthetic construct
  REGION 1..231: note = Fc K chain
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 142 - protein, 231 aa, synthetic construct
  REGION 1..231: note = Fc D chain
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVDGFYPSDIAVEWESNGQPENNYDTTPPVLDSDGSFFLYSDLTVDKSRWQQGNVFSCSVMHEALHNHYTQKSLSLSPG

SEQ ID NO: 143 - protein, 237 aa, synthetic construct
  REGION 1..237: note = Green Fluorescent protein
SKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 144 - protein, 237 aa, synthetic construct
  REGION 1..237: note = eGFP
SKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 145 - protein, 239 aa, synthetic construct
  REGION 1..239: note = eGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 146 - protein, 45 aa, synthetic construct
  REGION 1..45: note = Moiety: COMP
DLGPQMLRELQETNAALQDVRELLRQQVREITFLKNTVMECDACG

SEQ ID NO: 147 - protein, 33 aa, synthetic construct
  REGION 1..33: note = Moiety: VASP
DLQRVKQELLEEVKKELQKVKEEIIEAFVQELR

SEQ ID NO: 148 - protein, 40 aa, synthetic construct
  REGION 1..40: note = Moiety: ZZ12.6
DVQAISSTIQDLQDQVDSLAEVVLQNRRGLDLLTRSGGGS

SEQ ID NO: 149 - protein, 18 aa, synthetic construct
  REGION 1..18: note = HSA signal peptide
MKWVTFISLLFLFSSAYS

SEQ ID NO: 150 - protein, 22 aa, synthetic construct
  REGION 1..22: note = Ig kappa light chain
MDMRAPAGIFGFLLVLFPGYRS

SEQ ID NO: 151 - protein, 19 aa, synthetic construct
  REGION 1..19: note = human azurocidin preprotein signal sequence
MTRLTVLALLAGLLASSRA

SEQ ID NO: 152 - protein, 19 aa, synthetic construct
  REGION 1..19: note = IgG heavy chain signal peptide
MELGLSWIFLLAILKGVQC

SEQ ID NO: 153 - protein, 19 aa, synthetic construct
  REGION 1..19: note = IgG heavy chain signal peptide
MELGLRWVFLVAILEGVQC

SEQ ID NO: 154 - protein, 19 aa, synthetic construct
  REGION 1..19: note = IgG heavy chain signal peptide
MKHLWFFLLLVAAPRWVLS

SEQ ID NO: 155 - protein, 19 aa, synthetic construct
  REGION 1..19: note = IgG heavy chain signal peptide
MDWTWRILFLVAAATGAHS

SEQ ID NO: 156 - protein, 19 aa, synthetic construct
  REGION 1..19: note = IgG heavy chain signal peptide
MDWTWRFLFVVAAATGVQS

SEQ ID NO: 157 - protein, 19 aa, synthetic construct
  REGION 1..19: note = IgG heavy chain signal peptide
MEFGLSWLFLVAILKGVQC

SEQ ID NO: 158 - protein, 19 aa, synthetic construct
  REGION 1..19: note = IgG heavy chain signal peptide
MEFGLSWVFLVALFRGVQC

SEQ ID NO: 159 - protein, 26 aa, synthetic construct
  REGION 1..26: note = IgG heavy chain signal peptide
MDLLHKNMKHLWFFLLLVAAPRWVLS

SEQ ID NO: 160 - protein, 22 aa, synthetic construct
  REGION 1..22: note = IgG Kappa light chain signal sequence
MDMRVPAQLLGLLLLWLSGARC

SEQ ID NO: 161 - protein, 22 aa, synthetic construct
  REGION 1..22: note = IgG Kappa light chain signal sequence
MKYLLPTAAAGLLLLAAQPAMA

SEQ ID NO: 162 - protein, 17 aa, synthetic construct
  REGION 1..17: note = Gaussia luciferase
MGVKVLFALICIAVAEA

SEQ ID NO: 163 - protein, 18 aa, Homo sapiens
  REGION 1..18: note = Human albumin
MKWVTFISLLFLFSSAYS

SEQ ID NO: 164 - protein, 18 aa, Homo sapiens
  REGION 1..18: note = Human chymotrypsinogen
MAFLWLLSCWALLGTTFG

SEQ ID NO: 165 - protein, 14 aa, Homo sapiens
  REGION 1..14: note = Human interleukin-2
MQLLSCIALILALV

SEQ ID NO: 166 - protein, 15 aa, Homo sapiens
  REGION 1..15: note = Human trypsinogen-2
MNLLLILTFVAAAVA

SEQ ID NO: 167 - protein, 281 aa, synthetic construct
  REGION 1..281: note = TACI Fc
SLSCRKEQGEYYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 281 aa, synthetic construct
  REGION 1..281: note = TACI Fc
SLSCRKEQGEYYDHLLRDCISCASICGQHPKQCADFCENKLRSGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 281 aa, synthetic construct
  REGION 1..281: note = TACI Fc
SLSCRKEEGKFYDHLLQDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 170 - protein, 281 aa, synthetic construct
  REGION 1..281: note = TACI Fc
SLSCRKEEGKFYDHLLQDCISCASICGQHPKQCAYFCENKLRSGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 281 aa, synthetic construct
  REGION 1..281: note = TACI Fc
SLSCRKEQGKFYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 172 - protein, 281 aa, synthetic construct
  REGION 1..281: note = TACI Fc
SLSCRKEQGKFYDHLLRDCISCASICGQHPKQCAYFCENKLRSGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 227 aa, synthetic construct
  REGION 1..227: note = Fc
DKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 174 - protein, 232 aa, synthetic construct
  REGION 1..232: note = Fc
DPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 175 - protein, 227 aa, synthetic construct
  REGION 1..227: note = Fc variant
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 232 aa, synthetic construct
  REGION 1..232: note = Fc variant
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 43 aa, synthetic construct
  REGION 1..43: note = TACI variant
SLSCRKEEGKFYDHLLRDCISCASICGQHPKQCAYFCENKLRS

SEQ ID NO: 178 - protein, 43 aa, synthetic construct
  REGION 1..43: note = TACI variant
SLSCRKEEGEFYDHLLRDCISCASICGQHPKQCAYFCENKLRS

SEQ ID NO: 179 - protein, 43 aa, synthetic construct
  REGION 1..43: note = TACI variant
SLSCRKEEGKYYDHLLRDCISCASICGQHPKQCAYFCENKLRS

SEQ ID NO: 180 - protein, 43 aa, synthetic construct
  REGION 1..43: note = TACI variant
SLSCRKEEGKFYDHLLRDCISCASICGQHPKQCDYFCENKLRS

SEQ ID NO: 181 - protein, 43 aa, synthetic construct
  REGION 1..43: note = TACI variant
SLSCRKEEGKFYDHLLRDCISCASICGQHPKQCADFCENKLRS

SEQ ID NO: 182 - protein, 43 aa, synthetic construct
  REGION 1..43: note = TACI variant
SLSCRKEQGEYYDHLLQDCISCASICGQHPKQCAYFCENKLRS

SEQ ID NO: 183 - protein, 43 aa, synthetic construct
  REGION 1..43: note = TACI variant
SLSCRKEQGKYYDHLLRDCISCASICGQHPKQCAYFCENKLRS

SEQ ID NO: 184 - protein, 43 aa, synthetic construct
  REGION 1..43: note = TACI variant
SLSCRKEQGKYYDHLLQDCISCASICGQHPKQCAYFCENKLRS

SEQ ID NO: 185 - protein, 43 aa, synthetic construct
  REGION 1..43: note = TACI variant
SLSCRKEQGKYYDHLLRDCISCASICGQHPKQCDYFCENKLRS

SEQ ID NO: 186 - protein, 43 aa, synthetic construct
  REGION 1..43: note = TACI variant
SLSCRKEQGKYYDHLLRDCISCASICGQHPKQCADFCENKLRS

SEQ ID NO: 187 - protein, 43 aa, synthetic construct
  REGION 1..43: note = TACI variant
SLSCRKEQGKFYDHLLQDCISCASICGQHPKQCDYFCENKLRS

SEQ ID NO: 188 - protein, 43 aa, synthetic construct
  REGION 1..43: note = TACI variant
SLSCRKEQGKFYDHLLQDCISCASICGQHPKQCADFCENKLRS

SEQ ID NO: 189 - protein, 43 aa, synthetic construct
  REGION 1..43: note = TACI variant
SLSCRKEQGKFYDHLLRDCISCASICGQHPKQCDDFCENKLRS

SEQ ID NO: 190 - protein, 43 aa, synthetic construct
  REGION 1..43: note = TACI variant
SLSCRKEQGKFYDHLLRDCISCASICGQHPKQCADFCENKLRS

SEQ ID NO: 191 - protein, 43 aa, synthetic construct
  REGION 1..43: note = TACI variant
SLSCRKEQGEYYDHLLRDCISCASICGQHPKQCAYFCENKLRS

SEQ ID NO: 192 - protein, 43 aa, synthetic construct
  REGION 1..43: note = TACI variant
SLSCRKEQGEFYDHLLRDCISCASICGQHPKQCADFCENKLRS

SEQ ID NO: 193 - protein, 227 aa, synthetic construct; identical to SEQ ID NO: 173
  REGION 1..227: note = Fc variant

SEQ ID NO: 194 - protein, 12 aa, synthetic construct
  REGION 1..12: note = linker
GSGGGGSGGGGS

SEQ ID NO: 195 - protein, 65 aa, synthetic construct
  REGION 1..65: note = linker
GGGGSGGGGSGGGGSGGGGSGGGGSGGGGSGGGGSGGGGSGGGGSGGGGSGGGGSGGGGSGGGGS

SEQ ID NO: 196 - protein, 577 aa, synthetic construct
  REGION 1..577: note = TACI FC
SLSCRKEQGKFYDHLLRDCISCASICGQHPKQCAYFCENKLRSGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSGGGGSGGGGSGGGGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 197 - protein, 281 aa, synthetic construct; identical to SEQ ID NO: 171
  REGION 1..281: note = TACI FC

SEQ ID NO: 198 - protein, 344 aa, synthetic construct
  REGION 1..344: note = TACI FC
SLSCRKEQGKFYDHLLRDCISCASICGQHPKQCAYFCENKLRSGGGGSGGGGSGGGGSGGGGSSLSCRKEQGKFYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 199 - protein, 577 aa, synthetic construct
  REGION 1..577: note = TACI FC
SLSCRKEQGEYYDHLLRDCISCASICGQHPKQCADFCENKLRSGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSGGGGSGGGGSGGGGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 200 - protein, 281 aa, synthetic construct; identical to SEQ ID NO: 167
  REGION 1..281: note = TACI FC

SEQ ID NO: 201 - protein, 344 aa, synthetic construct
  REGION 1..344: note = TACI FC
SLSCRKEQGEYYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSLSCRKEQGEYYDHLLRDCISCASICGQHPKQCADFCENKLRS

SEQ ID NO: 202 - protein, 344 aa, synthetic construct
  REGION 1..344: note = TACI FC
SLSCRKEQGKFYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSLSCRKEQGKFYDHLLRDCISCASICGQHPKQCAYFCENKLRS

SEQ ID NO: 203 - protein, 616 aa, synthetic construct
  REGION 1..616: note = TACI FC
VAMRSCPEEQYWDPLLGTCMSCKTICNHQSQRTCAAFCRSLSCRKEQGKFYDHLLRDCISCASICGQHPKQCAYFCENKLRSGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSGGGGSGGGGSGGGGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 204 - protein, 313 aa, synthetic construct; identical to SEQ ID NO: 132
  REGION 1..313: note = TACI FC

SEQ ID NO: 205 - protein, 640 aa, synthetic construct
  REGION 1..640: note = TACI FC
SRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSGGGGSGGGGSGGGGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 206 - protein, 333 aa, synthetic construct
  REGION 1..333: note = TACI FC
SRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - DNA, 1731 bp, synthetic construct
  misc_feature 1..1731: note = TACI FC
  source 1..1731: mol_type = other DNA
agtctctcttgcaggaaagagcaaggcaaattttacgaccatcttttgcgagactgtataagctgtgcgtctatttgtggacaacaccctaaacaatgtgcttatttctgtgaaaataagcttcgatctggatccggtggtggcgggtcagggggaggtggaagcgagcccaagtcctcagataagactcatacttgccccccctgtcccgcccctgaggccgaaggcgcaccgtcagtgtttttgttcccaccaaagcctaaggacacactgatgataagcagaacacctgaggtaacctgtgttgttgtggatgtaagccacgaagatccggaggttaagtttaactggtacgtggacggtgtagaagtccataatgctaagacgaagccgagggaggaacaatacaactccacgtatagagtggtctccgtcttgaccgtactccatcaagactggctcaacgggaaagagtacaagtgtaaagtctctaacaaagctcttcctgcaccgattgagaaaaccatatctaaagccaagggacaaccgagagaaccacaggtttacacgctcccccccagtagagacgaacttacaaaaaaccaggtcagtcttacctgcctcgtcaaaggcttctacccctctgacatcgctgttgagtgggaatctaacggccaacctgagaacaattacaaaacaaccccgcctgtccttgactcagacggttccttttttctttacagcaagctgaccgtcgacaaatcacggtggcaacaaggcaatgtgttttcctgttccgtgatgcacgaggcactgcacaaccactacactcaaaaatccctttccctttccccagggggaggtggagggagcggtggaggtggtagcgggggtggaggctcaggtggtgggggttccggcggtggcggaagtggaggcggtggctctggtggtggcggatctggcggaggaggcagcggcggaggtgggtctgggggtggaggctccggaggcgggggaagcggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 208 - DNA, 843 bp, synthetic construct
  misc_feature 1..843: note = TACI FC
  source 1..843: mol_type = other DNA
agtctctcttgcaggaaagagcaaggcaaattttacgaccatcttttgcgagactgtataagctgtgcgtctatttgtggacaacaccctaaacaatgtgcttatttctgtgaaaataagcttcgatctggatccggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 209 - DNA, 1032 bp, synthetic construct
  misc_feature 1..1032: note = TACI FC
  source 1..1032: mol_type = other DNA
agtctctcttgcaggaaagagcaaggcaaattttacgaccatcttttgcgagactgtataagctgtgcgtctatttgtggacaacaccctaaacaatgtgcttatttctgtgaaaataagcttcgatctgggggtggcggatcaggaggtggcggttcaggcggaggaggttctgggggtgggggttcttcattgtcctgcagaaaggaacaggggaaattttacgatcacttgcttagagattgtataagctgcgcgagcatttgcgggcaacaccctaaacagtgtgcgtatttctgcgagaataaactccggtctggatccggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 210 - DNA, 1731 bp, synthetic construct
  misc_feature 1..1731: note = TACI FC
  source 1..1731: mol_type = other DNA
agtctctcttgcaggaaagagcaaggcgaatattacgaccatcttttgcgagactgtataagctgtgcgtctatttgtggacaacaccctaaacaatgtgctgatttctgtgaaaataagcttcgatctggatccggtggtggcgggtcagggggaggtggaagcgagcccaagtcctcagataagactcatacttgccccccctgtcccgcccctgaggccgaaggcgcaccgtcagtgtttttgttcccaccaaagcctaaggacacactgatgataagcagaacacctgaggtaacctgtgttgttgtggatgtaagccacgaagatccggaggttaagtttaactggtacgtggacggtgtagaagtccataatgctaagacgaagccgagggaggaacaatacaactccacgtatagagtggtctccgtcttgaccgtactccatcaagactggctcaacgggaaagagtacaagtgtaaagtctctaacaaagctcttcctgcaccgattgagaaaaccatatctaaagccaagggacaaccgagagaaccacaggtttacacgctcccccccagtagagacgaacttacaaaaaaccaggtcagtcttacctgcctcgtcaaaggcttctacccctctgacatcgctgttgagtgggaatctaacggccaacctgagaacaattacaaaacaaccccgcctgtccttgactcagacggttccttttttctttacagcaagctgaccgtcgacaaatcacggtggcaacaaggcaatgtgttttcctgttccgtgatgcacgaggcactgcacaaccactacactcaaaaatccctttccctttccccagggggaggtggagggagcggtggaggtggtagcgggggtggaggctcaggtggtgggggttccggcggtggcggaagtggaggcggtggctctggtggtggcggatctggcggaggaggcagcggcggaggtgggtctgggggtggaggctccggaggcgggggaagcggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 211 - DNA, 843 bp, synthetic construct
  misc_feature 1..843: note = TACI FC
  source 1..843: mol_type = other DNA
agtctctcttgcaggaaagagcaaggcgaatattacgaccatcttttgcgagactgtataagctgtgcgtctatttgtggacaacaccctaaacaatgtgctgatttctgtgaaaataagcttcgatctggatccggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 212 - DNA, 1032 bp, synthetic construct
  misc_feature 1..1032: note = TACI FC
  source 1..1032: mol_type = other DNA
agtctctcttgcaggaaagagcaaggcgaatattacgaccatcttttgcgagactgtataagctgtgcgtctatttgtggacaacaccctaaacaatgtgctgatttctgtgaaaataagcttcgatctggatccggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggggggggtggcggatcaggaggtggcggttcaggcggaggaggttctgggggtgggggttcttcattgtcctgcagaaaggaacagggggagtattacgatcacttgcttagagattgtataagctgcgcgagcatttgcgggcaacaccctaaacagtgtgcggatttctgcgagaataaactccggtct

SEQ ID NO: 213 - DNA, 1032 bp, synthetic construct
  misc_feature 1..1032: note = TACI FC
  source 1..1032: mol_type = other DNA
agtctctcttgcaggaaagagcaaggcaaattttacgaccatcttttgcgagactgtataagctgtgcgtctatttgtggacaacaccctaaacaatgtgcttatttctgtgaaaataagcttcgatctggatccggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggggggggtggcggatcaggaggtggcggttcaggcggaggaggttctgggggtgggggttcttcattgtcctgcagaaaggaacaggggaaattttacgatcacttgcttagagattgtataagctgcgcgagcatttgcgggcaacaccctaaacagtgtgcgtatttctgcgagaataaactccggtct

SEQ ID NO: 214 - DNA, 1848 bp, synthetic construct
  misc_feature 1..1848: note = TACI FC
  source 1..1848: mol_type = other DNA
gtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgcttatttctgtgaaaataagcttcgatctggatccggtggtggcgggtcagggggaggtggaagcgagcccaagtcctcagataagactcatacttgccccccctgtcccgcccctgaggccgaaggcgcaccgtcagtgtttttgttcccaccaaagcctaaggacacactgatgataagcagaacacctgaggtaacctgtgttgttgtggatgtaagccacgaagatccggaggttaagtttaactggtacgtggacggtgtagaagtccataatgctaagacgaagccgagggaggaacaatacaactccacgtatagagtggtctccgtcttgaccgtactccatcaagactggctcaacgggaaagagtacaagtgtaaagtctctaacaaagctcttcctgcaccgattgagaaaaccatatctaaagccaagggacaaccgagagaaccacaggtttacacgctcccccccagtagagacgaacttacaaaaaaccaggtcagtcttacctgcctcgtcaaaggcttctacccctctgacatcgctgttgagtgggaatctaacggccaacctgagaacaattacaaaacaaccccgcctgtccttgactcagacggttccttttttctttacagcaagctgaccgtcgacaaatcacggtggcaacaaggcaatgtgttttcctgttccgtgatgcacgaggcactgcacaaccactacactcaaaaatccctttccctttccccagggggaggtggagggagcggtggaggtggtagcgggggtggaggctcaggtggtgggggttccggcggtggcggaagtggaggcggtggctctggtggtggcggatctggcggaggaggcagcggcggaggtgggtctgggggtggaggctccggaggcgggggaagcggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 215 - DNA, 939 bp, synthetic construct
  misc_feature 1..939: note = TACI FC
  source 1..939: mol_type = other DNA
gcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgcttatttctgtgaaaataagcttcgatctgagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccgagctccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccggggaag

SEQ ID NO: 216 - DNA, 1920 bp, synthetic construct
  misc_feature 1..1920: note = TACI FC
  source 1..1920: mol_type = other DNA
agccgtgtggaccaggaggagcgctttccacagggcctgtggacgggggtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgcttatttctgtgaaaataagcttcgatctcccgtgaacctgccacccgagctgggatccggtggtggcgggtcagggggaggtggaagcgagcccaagtcctcagataagactcatacttgccccccctgtcccgcccctgaggccgaaggcgcaccgtcagtgtttttgttcccaccaaagcctaaggacacactgatgataagcagaacacctgaggtaacctgtgttgttgtggatgtaagccacgaagatccggaggttaagtttaactggtacgtggacggtgtagaagtccataatgctaagacgaagccgagggaggaacaatacaactccacgtatagagtggtctccgtcttgaccgtactccatcaagactggctcaacgggaaagagtacaagtgtaaagtctctaacaaagctcttcctgcaccgattgagaaaaccatatctaaagccaagggacaaccgagagaaccacaggtttacacgctcccccccagtagagacgaacttacaaaaaaccaggtcagtcttacctgcctcgtcaaaggcttctacccctctgacatcgctgttgagtgggaatctaacggccaacctgagaacaattacaaaacaaccccgcctgtccttgactcagacggttccttttttctttacagcaagctgaccgtcgacaaatcacggtggcaacaaggcaatgtgttttcctgttccgtgatgcacgaggcactgcacaaccactacactcaaaaatccctttccctttccccagggggaggtggagggagcggtggaggtggtagcgggggtggaggctcaggtggtgggggttccggcggtggcggaagtggaggcggtggctctggtggtggcggatctggcggaggaggcagcggcggaggtgggtctgggggtggaggctccggaggcgggggaagcggtggaggagggtcagagcccaaaagctccgacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccggcacccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccgggg

SEQ ID NO: 217 - DNA, 999 bp, synthetic construct
  misc_feature 1..999: note = TACI FC
  source 1..999: mol_type = other DNA
agccgtgtggaccaggaggagcgctttccacagggcctgtggacgggggtggcaatgcgctcatgcccagaggaacaatattgggatccgcttcttgggacgtgtatgagctgcaagaccatctgtaatcatcaatcccaaaggacatgcgcagctttctgcaggagtctctcttgcaggaaagagcaaggcaaattttacgaccatcttttgcgagactgtataagctgtgcgtctatttgtggacaacaccctaaacaatgtgcttatttctgtgaaaataagcttcgatctcccgtgaacctgccacccgagctggacaagactcacacatgccccccttgtccagcgcctgaagctgagggtgcgccctctgtcttccttttcccccctaagccgaaagataccctgatgatctcccgcactcccgaagtcacatgtgttgttgtcgacgtatctcatgaagatcctgaggtgaaattcaactggtatgtagacggggtcgaagttcataatgctaagactaagccacgagaagagcaatacaactcaacgtatcgggtggtgagcgttctgacggttctgcaccaagattggcttaatggaaaagagtataagtgcaaggtgtccaacaaggctcttccgagctccatcgaaaagacgatttccaaagcgaaaggccaacctagggaaccgcaagtttacactttgcccccgtcaagagacgaacttaccaagaatcaagtttccctgacgtgccttgtgaagggcttctaccctagcgatatagcagttgagtgggaatctaacggccagcccgaaaataattataagactactccgcccgtgctggacagtgatggttcatttttcctgtattcaaaactcactgtggacaaatctagatggcagcagggtaatgtgttctcttgttcagttatgcacgaggcattgcacaatcactatacgcaaaaaagtttgtctctctctccggggaag

SEQ ID NO: 218 - protein, 522 aa, synthetic construct
  REGION 1..522: note = FC VARIANT
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSGGGGSGGGGSGGGGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 219 - protein, 222 aa, synthetic construct
  REGION 1..222: note = FC VARIANT
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 220 - protein, 228 aa, synthetic construct
  REGION 1..228: note = FC VARIANT
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 221 - protein, 226 aa, synthetic construct
  REGION 1..226: note = FC VARIANT
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 222 - protein, 281 aa, synthetic construct
  REGION 1..281: note = TACI VARIANT
SLSCRKEEGKFYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 223 - protein, 281 aa, synthetic construct
  REGION 1..281: note = TACI VARIANT
SLSCRKEEGEFYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 224 - protein, 281 aa, synthetic construct
  REGION 1..281: note = TACI FC
SLSCRKEEGKYYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 225 - protein, 281 aa, synthetic construct
  REGION 1..281: note = TACI FC
SLSCRKEEGKFYDHLLRDCISCASICGQHPKQCDYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 226 - protein, 281 aa, synthetic construct
  REGION 1..281: note = TACI FC
SLSCRKEEGKFYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 227 - protein, 281 aa, synthetic construct
  REGION 1..281: note = TACI FC
SLSCRKEQGEYYDHLLQDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 228 - protein, 281 aa, synthetic construct
  REGION 1..281: note = TACI FC
SLSCRKEQGKYYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 229 - protein, 281 aa, synthetic construct
  REGION 1..281: note = TACI FC
SLSCRKEQGKYYDHLLQDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 230 - protein, 281 aa, synthetic construct
  REGION 1..281: note = TACI FC
SLSCRKEQGKYYDHLLRDCISCASICGQHPKQCDYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 231 - protein, 281 aa, synthetic construct
  REGION 1..281: note = TACI FC
SLSCRKEQGKYYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 232 - protein, 281 aa, synthetic construct
  REGION 1..281: note = TACI FC
SLSCRKEQGKFYDHLLQDCISCASICGQHPKQCDYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 233 - protein, 281 aa, synthetic construct
  REGION 1..281: note = TACI FC
SLSCRKEQGKFYDHLLQDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 234 - protein, 281 aa, synthetic construct
  REGION 1..281: note = TACI FC
SLSCRKEQGKFYDHLLRDCISCASICGQHPKQCDDFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 235 - protein, 281 aa, synthetic construct
  REGION 1..281: note = TACI FC
SLSCRKEQGKFYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 236 - protein, 281 aa, synthetic construct
  REGION 1..281: note = TACI FC
SLSCRKEQGEYYDHLLRDCISCASICGQHPKQCAYFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 237 - protein, 281 aa, synthetic construct
  REGION 1..281: note = TACI FC
SLSCRKEQGEFYDHLLRDCISCASICGQHPKQCADFCENKLRSGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 238 - protein, 5 aa, synthetic construct
  REGION 1..5: note = Hinge
EPKSS

SEQ ID NO: 239 - protein, 5 aa, synthetic construct
  REGION 1..5: note = hinge
EPKSC

SEQ ID NO: 240 - protein, 333 aa, synthetic construct
  REGION 1..333: note = TACI 13-118 -Fc - 81
SRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 241 - protein, 333 aa, synthetic construct; identical to SEQ ID NO: 206
  REGION 1..333: note = TACI 13-118-Fc